(19) United States
(12) Patent Application Publication
(10) Pub. No.: US 2022/0093870 A1
(43) Pub. Date: Mar. 24, 2022

(54) COMPOUND FOR ORGANIC ELECTRONIC ELEMENT, ORGANIC ELECTRONIC ELEMENT USING SAME AND ELECTRONIC DEVICE THEREFOR

(71) Applicant: DUK SAN NEOLUX CO., LTD., Cheonan-si (KR)

(72) Inventors: Soung Yun MUN, Cheonan-si (KR); Jae Taek KWON, Cheonan-si (KR); Jong Gwang PARK, Cheonan-si (KR); Yun Suk LEE, Seongnam-si (KR); Won Sam KIM, Hwaseong-si (KR); Seul Gi KIM, Daejeon (KR); Jin Bae JEON, Cheonan-si (KR); Jung Hwan PARK, Hwaseong-si (KR)

(73) Assignee: DUK SAN NEOLUX CO., LTD., Cheonan-si (KR)

(21) Appl. No.: 17/399,676

(22) Filed: Aug. 11, 2021

Related U.S. Application Data
(62) Division of application No. 16/304,346, filed on Nov. 26, 2018.

(30) Foreign Application Priority Data
May 26, 2016 (KR) 10-2016-0065005
Jul. 4, 2016 (KR) 10-2016-0084282
May 19, 2017 (KR) 10-2017-0062429

(51) Int. Cl. H01L 51/00; C07D 307/91; C07D 333/76; C07D 409/12; H01L 51/50; C07D 209/82; C07C 211/54; C09K 11/06

(52) U.S. Cl. CPC H01L 51/0061; H01L 51/5088; C07D 333/76; C07D 409/12; H01L 51/0054; H01L 51/0058; H01L 51/006; H01L 51/0067; H01L 51/0072; H01L 51/5016; H01L 51/50; C07D 209/82; H01L 51/00; C07C 211/54; C09K 11/06; H01L 51/0073; H01L 51/0074; H01L 51/5012; H01L 51/5056; C07D 307/91

(57) ABSTRACT

Provided are an organic electronic element and an electronic device therefor, the organic electronic element having a mixture of a compound according to the present invention used as material for an organic layer thereof, thereby enabling the achievement of high light-emitting efficiency and low driving voltage of the organic electronic element, and enabling the life of the element to be greatly extended.

### BACKGROUND

### Technical Field

The present invention relates to compound for organic electronic element, organic electronic element using the same, and an electronic device thereof.

### Background Art

In general, organic light emitting phenomenon refers to a phenomenon that converts electronic energy into light energy by using an organic material. An organic electronic element using an organic light emitting phenomenon usually has a structure including an anode, a cathode, and an organic material layer interposed therebetween. Here, in order to increase the efficiency and stability of the organic electronic element, the organic material layer is often composed of a multi-layered structure composed of different materials, and for example, may include a hole injection layer, a hole transport layer, an emitting layer, an electron transport layer, an electron injection layer and the like.

A material used as an organic material layer in an organic electronic element may be classified into a light emitting material and a charge transport material, such as a hole injection material, a hole transport material, an electron transport material, an electron injection material and the like depending on its function.

In the case of a polycyclic compound containing a heteroatom, the difference in properties according to the material structure is so large that it is applied to various layers as a material of an organic electronic element. In particular, it has characteristics of different band gaps (HOMO, LUMO), electronical characteristics, chemical properties, and physical properties depending on the number of rings, fused positions and the type and arrangement of heteroatoms, therefore application development for layers of various organic electronic elements using the same has been progressed.

As a representative example thereof, in the following Patent Documents 1 to 4, the performance of the 5-membered cyclic compound in the polycyclic compound has been reported depending on the hetero type, arrangement, substituent type, fused position, and the like.
[Patent Document 1]: U.S. Pat. No. 5,843,607
[Patent Document 2]: Japanese Laid-Open Patent Publication No. 1999-162650
[Patent Document 3]: Korean Published Patent Application No. 2008-0085000
[Patent Document 4]: US Patent Publication No. 2010-0187977
[Patent Document 5]: Korean Published Patent Application No. 2011-0018340
[Patent Document 6]: Korean Published Patent Application No. 2009-0057711

Patent Documents 1 and 2 disclose an embodiment in which the indolecarbazole core in which the hetero atom in the 5-membered cyclic compound is composed only of nitrogen (N) is used, and an aryl group substituted or unsubstituted in N of indolocarbazole is used. However, in the prior invention 1, there exists only a simple aryl group substituted or unsubstituted with an alkyl group, an amino group, an alkoxy group, or the like as a substituent, so that the effect of the substituents of the polycyclic compounds was very poor to prove, and only the use as a hole transport material is described, and the use thereof as a phosphorescent host material is not described.

Patent Documents 3 and 4 disclose a compound in which pyridine, pyrimidine, triazine or the like containing an aryl group and N, respectively, were substituted for an indolecarbazole core having a hetero atom N in the same 5-membered cyclic compound as in the above Patent Documents 1 and 2, however only the use examples for phosphorescent green host materials are described, and the performance for other heterocyclic compounds substituted for indolecarbazole core is not described.

In Patent Documents 5, Nitrogen (N), oxygen (O), sulfur (S), carbon and the like are described as heteroatom in the 5-membered cyclic compound, however there are only examples using the same heteroatom in the performance measurement data, the performance characteristics of a 5-membered cyclic compound containing a different heteroatom could not be confirmed.

Therefore, the patent document does not disclose solutions to low charge carrier mobility and low oxidation stability of a 5-membered cyclic compound containing same heteroatom.

When the 5-membered cyclic compound molecules are generally laminated, as the adjacent π-electrons increase, they have a strong electronical interaction, and this is closely related to the charge carrier mobility, particularly, the same 5-membered cyclic compound of N—N type has an edge-to-face morphology as an order of arrangement of molecules when molecules are laminated, otherwise a different 5-membered cyclic compound with different heteroatoms has an antiparallel cofacial π-stacking structure in which the packing structure of the molecules is opposite to each other, so that the arrangement order of the molecules becomes face-to-face morphology. It is reported that the steric effect of the substituent substituted on the asymmetrically arranged hetero atom N as the cause of this laminated structure causes relatively high carrier mobility and high oxidation stability (Org. Lett. 2008, 10, 1199).

In Patent Document 6, an example of using as a fluorescent host material for various polycyclic compounds having 7 or more membered cyclic compounds has been reported.

As described above, the fused positions, the number of rings, the arrangement of heteroatoms, and characteristic change by type of the polycyclic compounds have not yet been sufficiently developed.

Particularly, in a phosphorescent organic electronic element using a phosphorescent dopant material, the LUMO and HOMO levels of the host material have a great influence on the efficiency and life span of the organic electronic element, this is because the charge balance control in the emitting layer, the quenching of the dopant, and the reduction in efficiency and life span due to light emission at the interface of the hole transport layer can be prevented, depending on whether electron and hole injection in the emitting layer can be efficiently controlled.

For fluorescent and phosphorescent host materials, recently we have been studying the increase of efficiency and life span of organic electronic elements using TADF (thermal activated delayed fluorescent), exciplex, etc., particularly, and many studies have been carried out to identify the energy transfer method from the host material to the dopant material.

Although there are various methods for identifying the energy transfer in the emitting layer for TADF (thermally activated delayed fluorescent) and exciplex, it can be easily confirmed by the PL lifetime (TRTP) measurement method.

The TRTP (Time Resolved Transient PL) measurement method is a method of observing a decay time over time after irradiating the host thin film with a pulsed light source, and therefore it is possible to identify the energy transfer method by observing the energy transfer and the lag time. The TRTP measurement can distinguish between fluorescence and phosphorescence, an energy transfer method in a mixed host material, an exciplex energy transfer method, and a TADF energy transfer method.

There are various factors affecting the efficiency and life span depending on the manner in which the energy is transferred from the host material to the dopant material, and the energy transfer method differs depending on the material, so that the development of stable and efficient host material for organic electronic element has not yet been sufficiently developed. Therefore, development of new materials is continuously required, and especially development of a host material for an emitting layer is urgently required.

### DETAILED DESCRIPTION OF THE INVENTION

### Summary

The present invention has been proposed in order to solve the problems of the phosphorescent host material, and an object of the present invention is, by controlling the HOMO level of a host material of a phosphorescent emitting organic electronic element including a phosphorescent dopant, to provide a compound capable of controlling charge balance and of improving efficiency and life span in an emitting layer, and an organic electronic element using the same and an electronic device thereof.

### Technical Solution

In order to control the efficient hole injection in the emitting layer of the phosphorescence emitting organic electronic element, by containing a specific second host material in combination with a specific first host material as a main component, it is possible to reduce the energy barrier of the emitting layer and the adjacent layer, the charge balance in the emitting layer is maximized, thereby providing high efficiency and high life of the organic electronic device.

The present invention provides an organic electronic element comprising a first electrode, a second electrode, and an organic material layer formed between the first electrode and the second electrode, wherein the organic material layer includes an emitting layer, wherein the emitting layer includes a first host compound represented by the following Formula (1) and a second host compound represented by the following Formula (2).

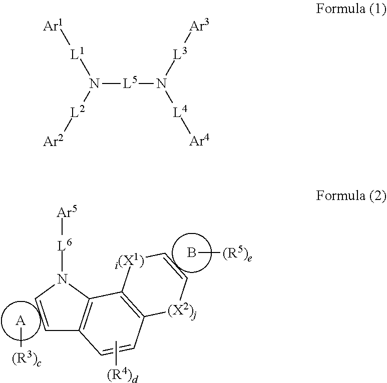

The present invention also provides an organic electronic element using the compound represented by the above Formulas and an electronic device thereof.

### Effects of the Invention

By using the mixture according to the present invention as a phosphorescent host material, it is possible to achieve a high luminous efficiency and a low driving voltage of an organic electric element, and the life span of the device can be greatly improved.

### BRIEF DESCRIPTION OF THE DRAWINGS

FIG. 1 is an illustration of an organic electroluminescent device according to the present invention.

| | |
|---|---|
| 100: organic electric element, | 110: substrate |
| 120: the first electrode(anode), | 130: the hole injection layer |
| 140: the hole transport layer, | 141: a buffer layer |
| 150: the emitting layer, | 151: the emitting auxiliary layer |
| 160: the electron transport layer, | 170: the electron injection layer |
| 180: the second electrode(cathode) | |

FIG. 2 and FIG. 3 show the 1H NMR analysis result of compound 2-76.

FIG. 4 and FIG. 5 show the 13C NMR analysis result of compound 2-76.

FIG. 6 show the 1H NMR analysis result of compound 3-6.

FIG. 7 shows the 13C NMR analysis result of compound 3-6.

FIG. 8 show the 1H NMR analysis result of compound 3-7.

FIG. 9 shows the 13C NMR analysis result of compound 3-7.

FIG. 10 show the 1H NMR analysis result of compound 3-8.

FIG. 11 shows the 13C NMR analysis result of compound 3-8.

FIG. 12 show the 13C NMR analysis result of compound 3-101.

FIG. 13 shows the 1H NMR analysis result of compound 3-101.

### DETAILED DESCRIPTION

Hereinafter, some embodiments of the present invention will be described in detail. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “linked” to another component, the component may be directly connected or connected to the other component, but another component may be “connected”, “coupled” or “linked” between each component.

As used in the specification and the accompanying claims, unless otherwise stated, the following is the meaning of the term as follows.

Unless otherwise stated, the term “halo” or “halogen”, as used herein, includes fluorine, bromine, chlorine, or iodine.

Unless otherwise stated, the term “alkyl” or “alkyl group”, as used herein, has a single bond of 1 to 60 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “haloalkyl” or “halogen alkyl”, as used herein, includes an alkyl group substituted with a halogen.

Unless otherwise stated, the term “heteroalkyl”, as used herein, means alkyl substituted one or more of carbon atoms consisting of an alkyl with hetero atom.

Unless otherwise stated, the term “alkenyl” or “alkynyl”, as used herein, has double or triple bonds of 2 to 60 carbon atoms, but is not limited thereto, and includes a linear or a branched chain group.

Unless otherwise stated, the term “cycloalkyl”, as used herein, means alkyl forming a ring having 3 to 60 carbon atoms, but is not limited thereto.

Unless otherwise stated, the term “alkoxyl group”, “alkoxy group” or “alkyloxy group”, as used herein, means an oxygen radical attached to an alkyl group, but is not limited thereto, and has 1 to 60 carbon atoms.

Unless otherwise stated, the term “alkenoxyl group”, “alkenoxy group”, “alkenyloxyl group” or “alkenyloxy group”, as used herein, means an oxygen radical attached to an alkenyl group, but is not limited thereto, and has 2 to 60 carbon atoms.

Unless otherwise stated, the term “aryloxyl group” or “aryloxy group”, as used herein, means an oxygen radical attached to an aryl group, but is not limited thereto, and has 6 to 60 carbon atoms.

Unless otherwise stated, the term “aryl group” or “arylene group”, as used herein, has 6 to 60 carbon atoms, but is not limited thereto. Herein, the aryl group or arylene group means a monocyclic and polycyclic aromatic group, and may also be formed in conjunction with an adjacent group. Examples of “aryl group” may include a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalkenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heteroalkyl”, as used herein, means alkyl containing one or more of hetero atoms. Unless otherwise stated, the term “heteroaryl group” or “heteroarylene group”, as used herein, means a C2 to C60 aryl containing one or more of hetero atoms or arylene group, but is not limited thereto, and includes at least one of monocyclic and polycyclic rings, and may also be formed in conjunction with an adjacent group.

Unless otherwise stated, the term “heterocyclic group”, as used herein, contains one or more heteroatoms, but is not limited thereto, has 2 to 60 carbon atoms, includes any one of monocyclic and polycyclic rings, and may include heteroaliphatic ring and/or heteroaromatic ring. Also, the heterocyclic group may also be formed in conjunction with an adjacent group. Unless otherwise stated, the term “heteroatom”, as used herein, represents at least one of N, O, S, P, or Si.

Also, the term “heterocyclic group” may include a ring containing SO₂ instead of carbon consisting of cycle. For example, “heterocyclic group” includes compound below.

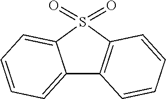 [SMILES: O=S1(=O)c2ccccc2-c2ccccc21]

Unless otherwise stated, the term “aliphatic”, as used herein, means an aliphatic hydrocarbon having 1 to 60 carbon atoms, and the term “aliphatic ring”, as used herein, means an aliphatic hydrocarbon ring having 3 to 60 carbon atoms.

Unless otherwise stated, the term “ring”, as used herein, means an aliphatic ring having 3 to 60 carbon atoms, or an aromatic ring having 6 to 60 carbon atoms, or a hetero ring having 2 to 60 carbon atoms, or a fused ring formed by the combination of them, and includes a saturated or unsaturated ring.

Other hetero compounds or hetero radicals other than the above-mentioned hetero compounds include, but are not limited thereto, one or more heteroatoms.

Unless otherwise stated, the term “carbonyl”, as used herein, is represented by —COR′, wherein R′ may be hydrogen, an alkyl having 1 to 20 carbon atoms, an aryl having 6 to 30 carbon atoms, a cycloalkyl having 3 to 30 carbon atoms, an alkenyl having 2 to 20 carbon atoms, an alkynyl having 2 to 20 carbon atoms, or the combination of these.

Unless otherwise stated, the term “ether”, as used herein, is represented by —R—O—R′, wherein R or R′ may be independently hydrogen, an alkyl having 1 to 20 carbon atoms, an aryl having 6 to 30 carbon atoms, a cycloalkyl having 3 to 30 carbon atoms, an alkenyl having 2 to 20 carbon atoms, an alkynyl having 2 to 20 carbon atoms, or the combination of these.

Unless otherwise stated, the term “substituted or unsubstituted”, as used herein, means that substitution is substituted by at least one substituent selected from the group consisting of, but is not limited thereto, deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiopen group, a C₆-C₂₀ arylthiopen group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group.

Unless otherwise expressly stated, the Formula used in the present invention, as used herein, is applied in the same manner as the substituent definition according to the definition of the exponent of the following Formula.

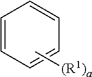

Wherein, when a is an integer of zero, the substituent R¹ is absent, that is, when a is 0, it means that hydrogen is bonded to all the carbons forming the benzene ring. In this case, the sign of the hydrogen bonded to the carbon may be omitted and the formula or compound may be described. When a is an integer of 1, the sole substituent R¹ is linked to any one of the carbon constituting the benzene ring, and when a is an integer of 2 or 3, they are respectively combined as follows, and when a is an integer from 4 to 6, it is bonded to the carbon of the benzene ring in a similar manner, and when a is an integer of 2 or more, R¹ may be the same or different from each other.

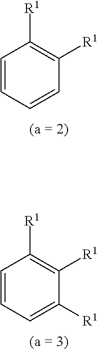

Unless otherwise expressly stated, the terms “ortho”, “meta”, and “para” used in the present invention refer to the substitution positions of all substituents, and the ortho position indicates the position of the substituent immediately adjacent to the compound, for example, when benzene is used, it means 1 or 2 position, and the meta position is the next substitution position of the neighbor substitution position, when benzene as an example stands for 1 or 3 position, and the para position is the next substitution position of the meta position, which means 1 and 4 position when benzene is taken as an example. A more detailed example of the substitution position is as follows, and it can be confirmed that the ortho-, and meta-position are substituted by non-linear type and para-positions are substituted by linear type.

### [Example of Ortho-Position]

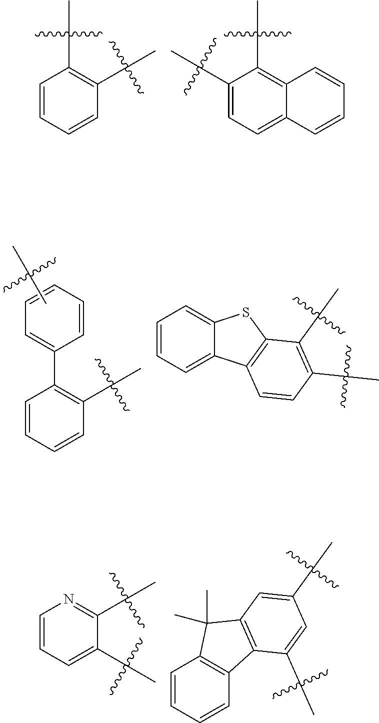

### [Example of Meta-Position]

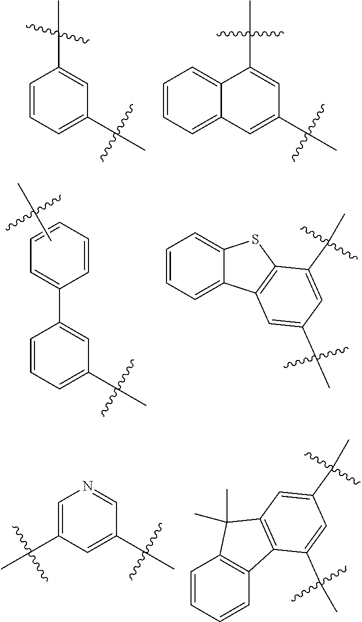

### [Example of Para-Position]

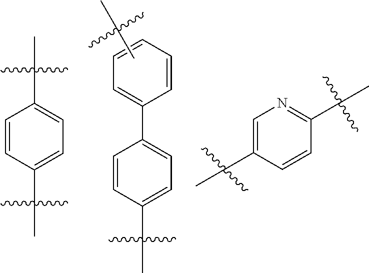

Hereinafter, a compound according to an aspect of the present invention and an organic electric element comprising the same will be described.

The present invention provides an organic electric element comprising a first electrode, a second electrode, and an organic material layer formed between the first electrode and the second electrode, wherein the organic material layer comprises an emitting layer, wherein the emitting layer comprises a first host compound represented by Formula (1) and a second host compound represented by Formula (2) as a phosphorescent light emitting layer.

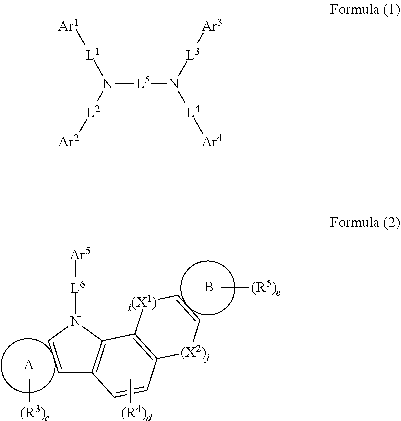

### {In Formulas (1) and (2),

1) Ar¹, Ar², Ar³, Ar⁴ and Ar⁵ are each independently selected from the group consisting of a C₆-C₆₀ aryl group; a fluorenyl group; a C₂-C₆₀ heterocyclic group including at least one hetero atom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; and -L′-N(Rₐ)(R_{b}); wherein, L′ is selected from the group consisting of a single bond; a C₆-C₆₀ arylene group; a fluorenylene group; a fused ring group of a C₃-C₆₀ aliphatic ring and a C₆-C₆₀ aromatic ring; and a C₂-C₆₀ heterocyclic; and Rₐ and R_{b} are each independently selected from the group consisting of a C₆-C₆₀ aryl group; a fluorenyl group; a fused ring group of a C₃-C₆₀ aliphatic ring and a C₆-C₆₀ aromatic ring; and a C₂-C₆₀ heterocyclic group containing at least one hetero atom of O, N, S, Si, or P, wherein Ar¹ and Ar² or Ar³ and Ar⁴ may be bonded to each other to form a ring,
2) c and e are an integer of 0 to 10, and d is an integer of 0 to 2,
3) R³, R⁴ and R⁵ are the same or different from each other, and are each independently selected from the group consisting of hydrogen; deuterium; halogen; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; and -L′-N(Rₐ)(R_{b}); or in case c, d and e are 2 or more, and R³, R⁴ and R⁵ are each in plural being the same or different, and a plurality of R³ or a plurality of R⁴ or a plurality of R⁵ may be bonded to each other to form a ring.
4) L¹, L², L³, L⁴, L⁵ and L⁶ are each independently selected from the group consisting of a single bond; a C₆-C₆₀ arylene group; and a fluorenylene group; a fused ring group of a C₃-C₆₀ aliphatic ring and a C₆-C₆₀ aromatic ring; and a C₂-C₆₀ heterocyclic group; provided that except when L⁵ is a single bond,
5) A and B are each independently a C₆-C₆₀ aryl group or a C₂-C₂₀ heterocyclic group, wherein both A and B are a substituted or unsubstituted C₆ aryl group (phenyl group), d is 2, and R⁴s are bonded to each other to form an aromatic ring or heterocycle,
6) i and j are 0 or 1, with the proviso that i+j is 1 or more, and when i or j is 0, it means a direct bond,
7) X₁ and X² are each independently N-L⁷-Ar⁶, O, S, or CR⁶R⁷;
   wherein L⁷ is the same as L¹ to L⁴ or L⁶, wherein Ar⁶ is the same as Ar¹ to Ar⁵,
   wherein R⁶ and R⁷ are each independently hydrogen; a C₆-C₆₀ aryl group; a fluorenyl group; a C₂-C₆₀ heterocyclic group; or a C₁-C₅₀ alkyl group; wherein R⁶ and R⁷ may combine to each other to form a spiro,
   wherein, the aryl group, fluorenyl group, arylene group, heterocyclic group, fluorenylene group, fused ring group, alkyl group, alkenyl group, alkoxy group and aryloxy group may be substituted with one or more substituents selected from the group consisting of deuterium; halogen; a silane group substituted or unsubstituted with C₁-C₂₀ alkyl group or C₆-C₂₀ aryl group; siloxane group; boron group; germanium group; cyano group; nitro group; -L′-N(Rₐ)(R_{b}); a C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂-C₂₀ heterocyclic group; C₃-C₂₀ cycloalkyl group; C₇-C₂₀ arylalkyl group; and C₈-C₂₀ arylalkenyl group; wherein the substituents may combine each other and form a saturated or unsaturated ring, wherein the term ‘ring’ means C₃-C₆₀ aliphatic ring or C₆-C₆₀ aromatic ring or a C₂-C₆₀ heterocyclic group or a fused ring formed by the combination of thereof and includes a saturated or unsaturated ring.}

In addition, the present invention provides the compounds represented by Formulas (1) and (2).

The present invention also provides an organic electric element comprising a compound represented by the following Formula (3) when AO and Are in Formula (1) form a ring.

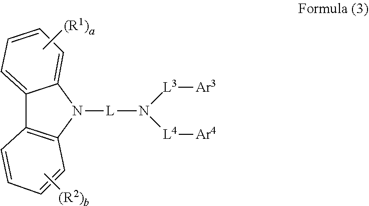

### {In Formula (3),

1) L³, L⁴, L⁵, Ara and Ar⁴ are the same as defined above,
2) a and b are each independently an integer of 0 to 4,
3) R¹ and R² are the same or different from each other, and are each independently selected from the group consisting of hydrogen; deuterium; halogen; a C₆-C₆₀ aryl group; a fluorenyl group; a C₂-C₆₀ heterocyclic group including at least one heteroatom of O, N, S, Si or P; a fused ring group of a C₃-C₂₀ aliphatic ring and a C₆-C₂₀ aromatic ring; a C₁-C₂₀ alkyl group; a C₂-C₂₀ alkenyl group; a C₂-C₂₀ alkynyl group; a C₁-C₂₀ alkoxyl group; a C₆-C₃₀ aryloxy group; and -L′-N(Rₐ)(R_{b}); or in case a and b are 2 or more, R¹ and R² are each in plural being the same or different, and a plurality of R¹ or a plurality of R² may be bonded to each other to form a ring.}

In Formula (1) of the present invention, L¹, L², L³, L⁴ and L⁵ are each independently any one of the following Formulas (A-1) to (A-13).

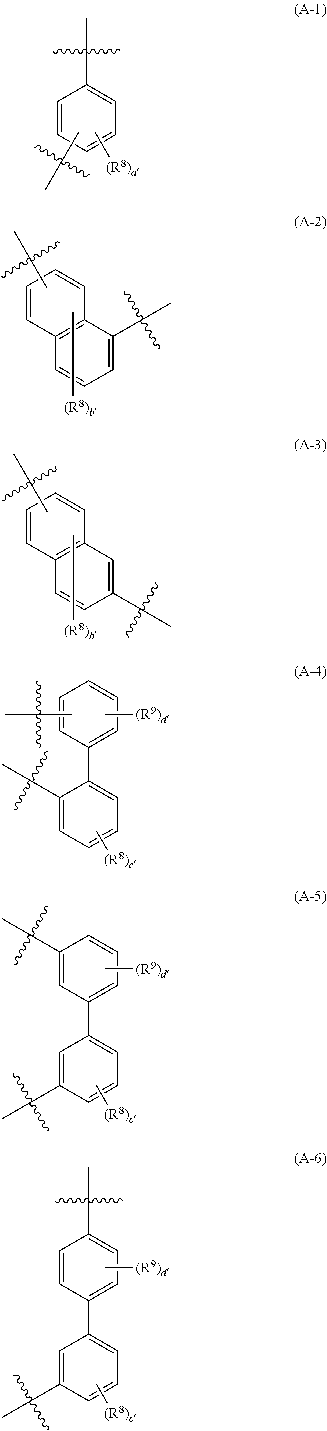

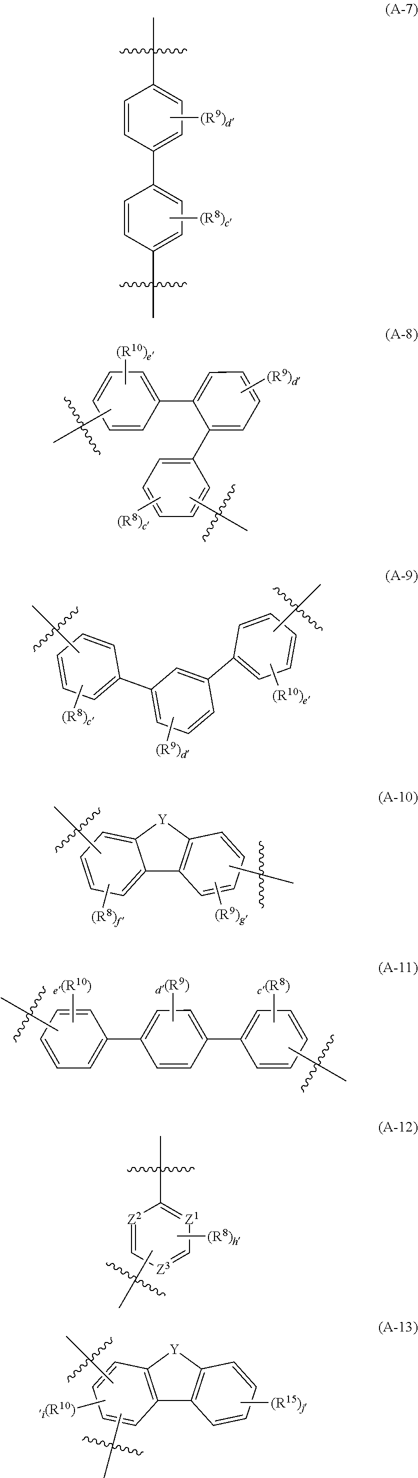

### {In Formulas (A-1) to (A-13),

1) a′, c′, d′ and e′ are an integer of 0 to 4; and b′ is an integer of 0 to 6; and f and g′ are an integer of 0 to 3; and h′ is an integer of 0 or 1; and i′ is an integer of 0 to 2, and j′ is an integer of 0 to 4,
2) R⁸, R⁹, R¹⁰ and R¹⁵ are the same or different from each other, and are each independently selected from the group consisting of hydrogen; deuterium; halogen; a C₆-C₂₀ aryl group; a fluorenyl group; a C₂-C₂₀ heterocyclic group including at least one heteroatom of O, N, S, Si or P; a fused ring group of a C₃-C₂₀ aliphatic ring and a C₆-C₂₀ aromatic ring; a C₁-C₂₀ alkyl group; a C₂-C₂₀ alkenyl group; a C₂-C₂₀ alkynyl group; a C₁-C₂₀ alkoxyl group; a C₆-C₃₀ aryloxy group; and -L′-N(Rₐ)(R_{b});
   wherein e′, f′, g′, i′ and j′ are 2 or more, R⁸, R⁹, R¹⁰ and R¹⁵ are the same or different from each other, and a plurality of R⁸ or a plurality of R⁹ or a plurality of R¹⁰ or a plurality of R¹⁵, two adjacent R⁸ and R⁹, or R⁹ and R¹⁰, or R¹⁰ and R¹⁵ may be bonded to form an aromatic ring or heteroaromatic ring,
3) Y is N-L⁸-Ar⁷, O, S or CR¹¹R¹²,
   Wherein L⁸ is the same as L¹ to L⁶ defined above, wherein Ar¹ is the same as Ar¹ to Ar⁵ defined above,
   Wherein R¹¹ and R¹² are the same as R⁶ and R⁷ defined above,
4) Z¹, Z² and Z³ are CR¹³ or N and at least one is N, and R¹³ is the same as R⁸ and R¹⁰ defined above.}

In Formula (1), L⁵ preferably comprises a compound represented by Formula (A-10), and the present invention also provides an organic electric device comprising the same. Formula (A-10) may be represented by the following Formulas C-1 to C-10, preferably Formulas C-2, C-3, C-4, C-6, C-7, C-9.

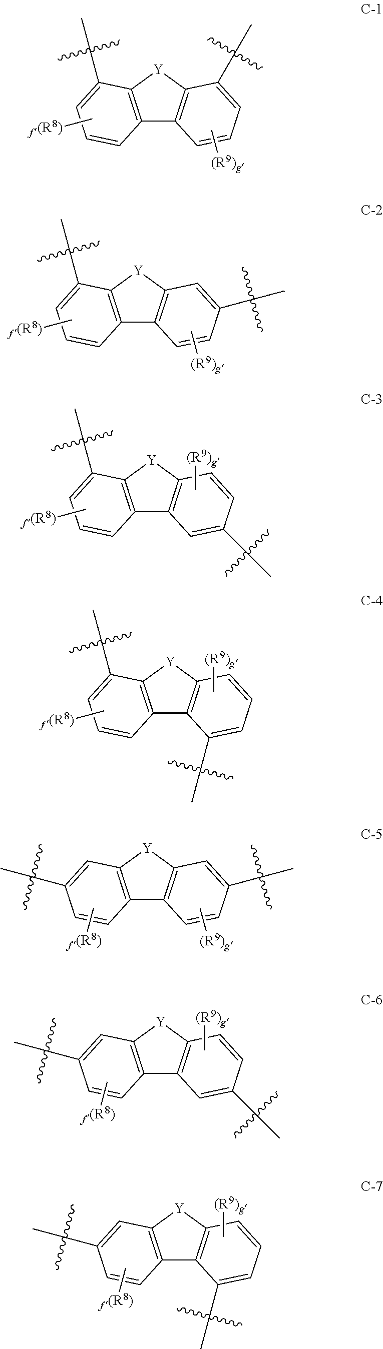

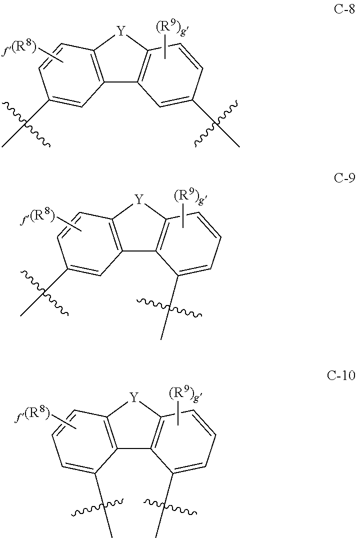

The first host compound represented by Formula (1) is represented by any one of the following Formulas (3-1) to (3-3).

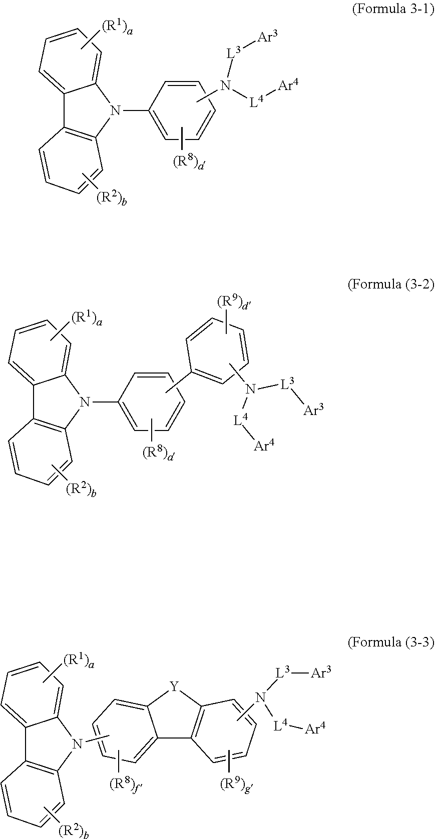

### {In Formulas (3-1) to (3-3),

R¹, R², R⁸, R⁹, a, b, a′, d′, f′, g′, L³, L⁴, Ara, Ar⁴ and Y are the same as defined above.}

The compound represented by Formula (1) of the present invention comprises a compound represented by the following Formula (3-4) or (3-5).

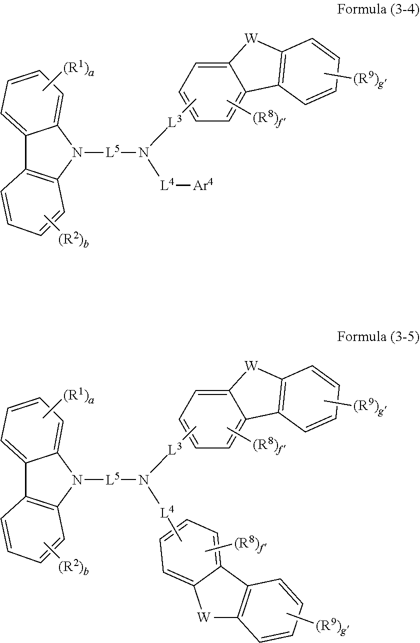

### {In Formulas (3-4) and (3-5),

1) Ar⁴, L³, L⁴, L⁵, R¹, R², R⁸, R⁹, a, b, f′ and g′ are the same as defined above,
2) W is as the definition of Y above.}

In one embodiment of the present invention, Ar³ and Ar⁴ in Formula (1) are all C₆-C₂₄ aryl groups, and more specifically, at least one of Ar³ and Ar⁴ of Formula (1) is dibenzothiophene or dibenzofuran.

In one embodiment of the present invention, the first host compound represented by Formula (1) is represented by any one of the following Formulas (3-6) to (3-19).

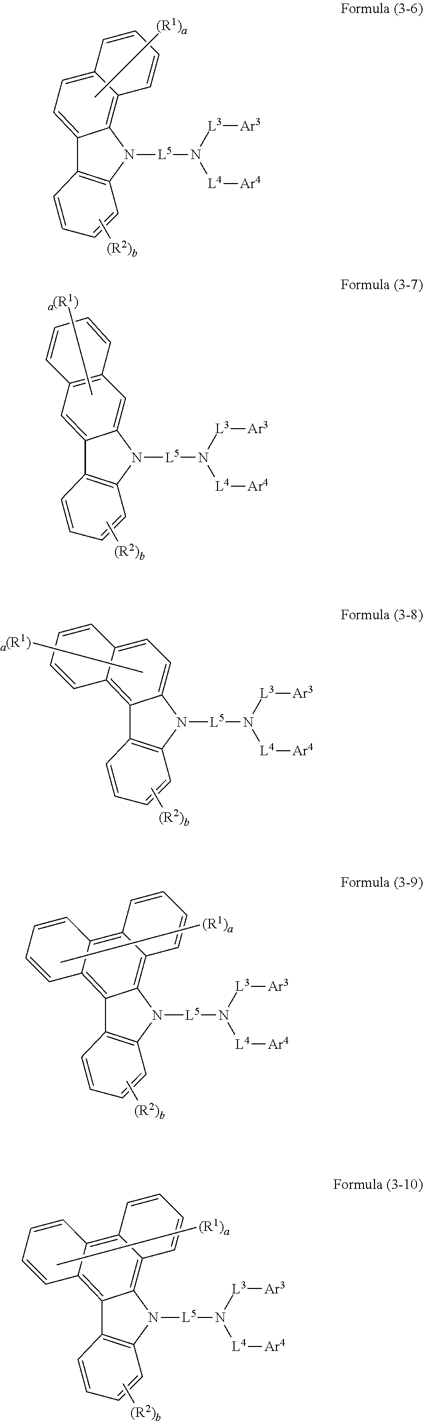

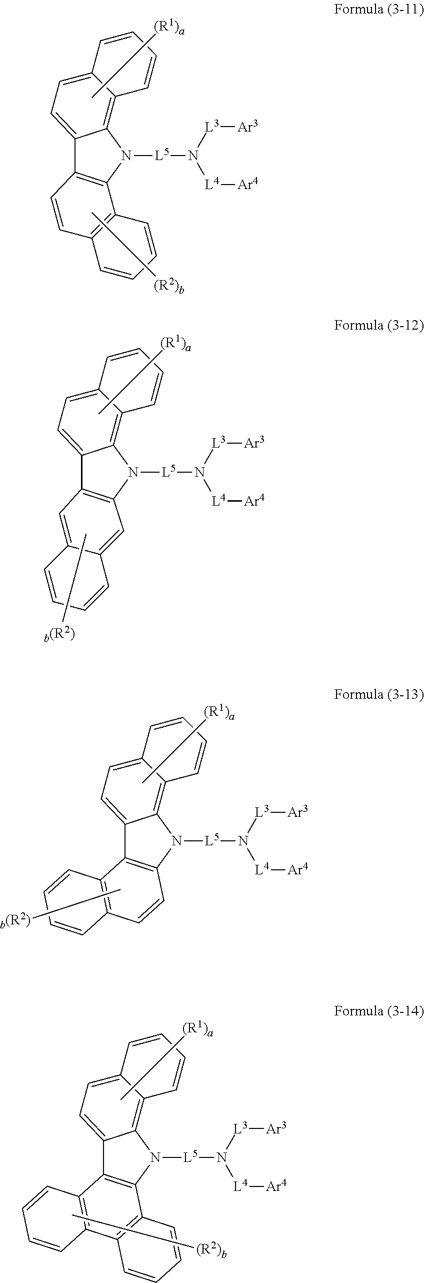

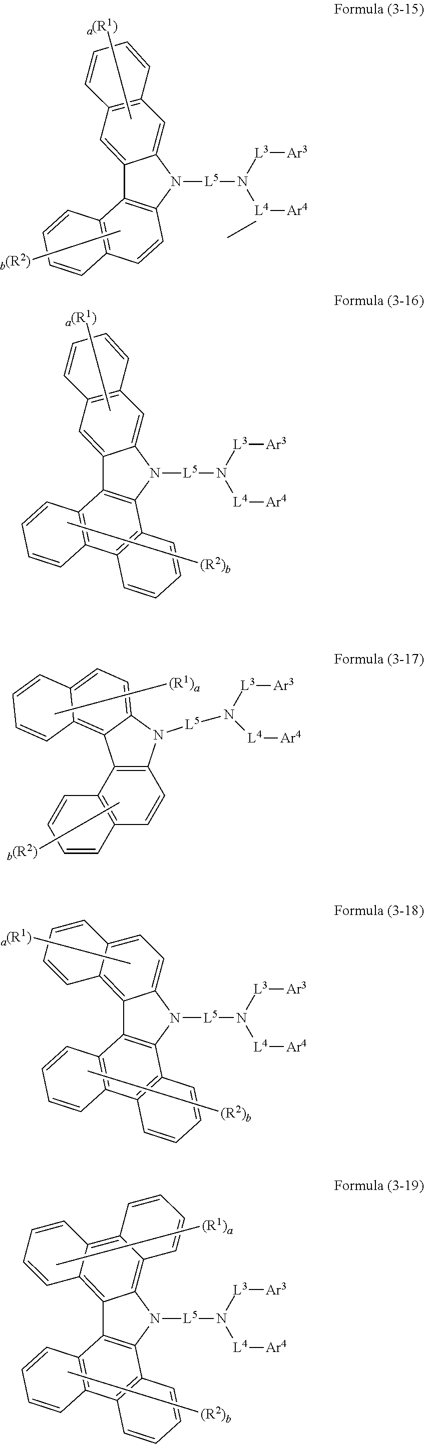

### {In Formulas (3-6) to (3-19),

L³, L⁴, L⁵, Ar³, Ar⁴, R¹ and R² are the same as defined above, and
a and b are any one of integers of 0 to 8.}

In another embodiment of the present invention, the first host compound represented by Formula (1) is represented by Formulas (3-20).

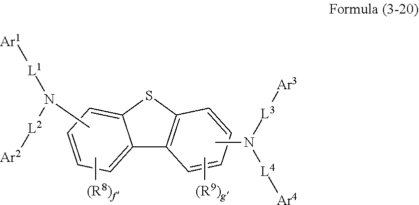

### {In Formula (3-20),

Ar¹, Ar², Ar³, Ar⁴, L¹, L², L³, L⁴, R⁸, R⁹, f′ and g′ are the same as defined above.}

Preferably, at least one of Ar¹, Ar², Ar³ and Ar⁴ in Formula (3-20) is dibenzothiophene or dibenzofuran.

In one embodiment of the present invention, at least one of L¹, L², L³, L⁴ and L⁵ iₙ Formula (1) is substituted with a meta position.

In another aspect, the present invention includes a compound wherein the second host compound represented by Formula (2) is represented by the following Formula (4) or (5).

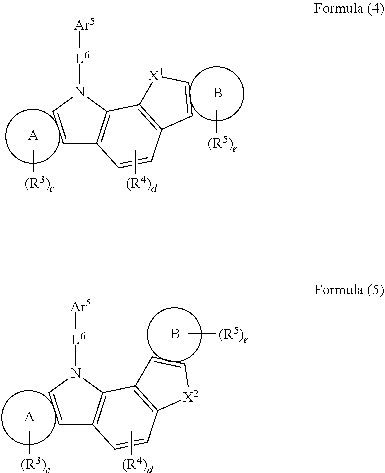

### {In Formulas (4) and (5),

R³, R⁴, R⁵, L⁶, Ar⁵, X², A, B, c, d, and e are the same as defined in Formula (2).}

The present invention also provides an organic electric element comprising a compound wherein A and B in Formula (2) are selected from the group consisting of the following Formulas (B-1) to (B-71.

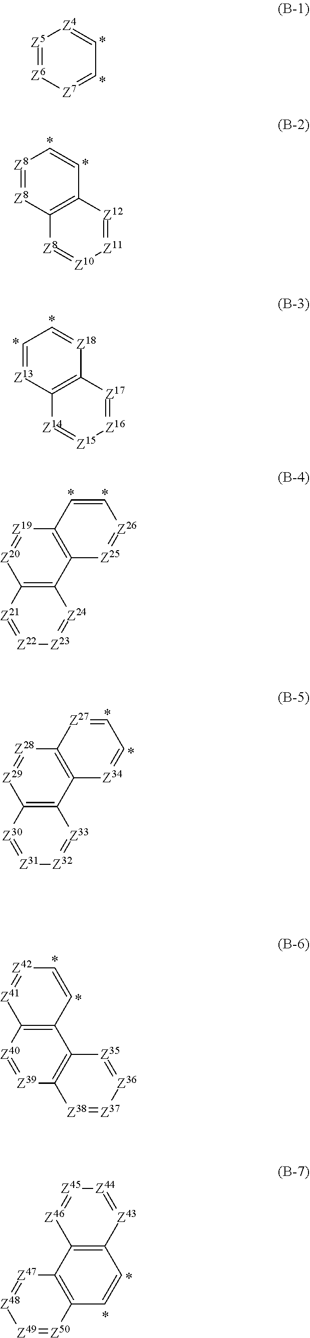

### {In Formulas (B-1) to (B-7),

1) Z⁴ to Z⁵⁰ are CR¹⁴ or N,
2) R¹⁴ is the same as R³ to R⁵ defined above,
3) * indicates the position to be condensed.}

As another example, the present invention provides a compound wherein the second host compound represented by Formula (2) includes a compound represented by any of the following Formulas (4-1) to (4-36).

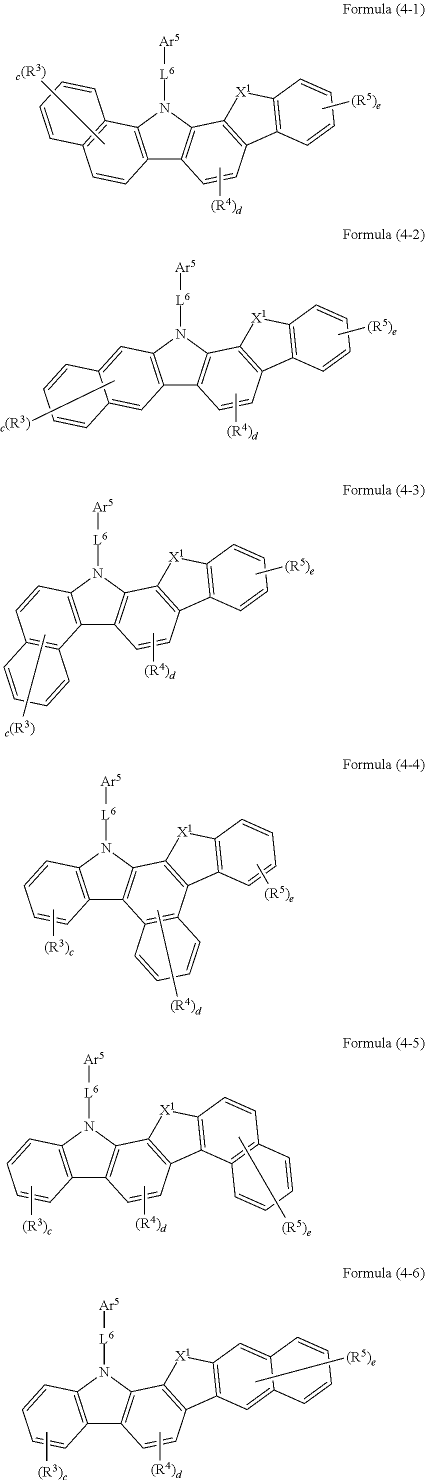

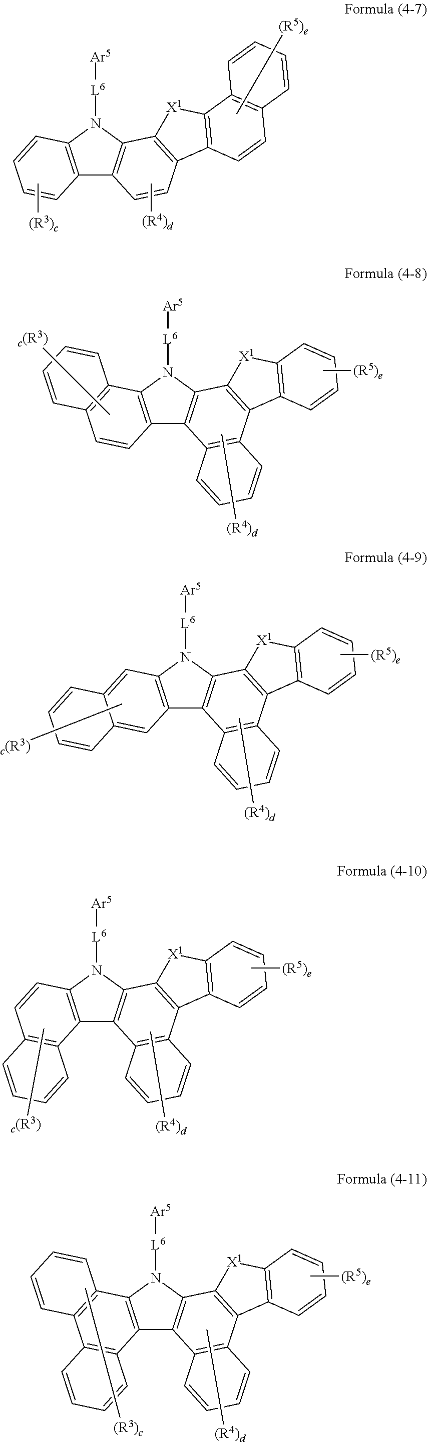

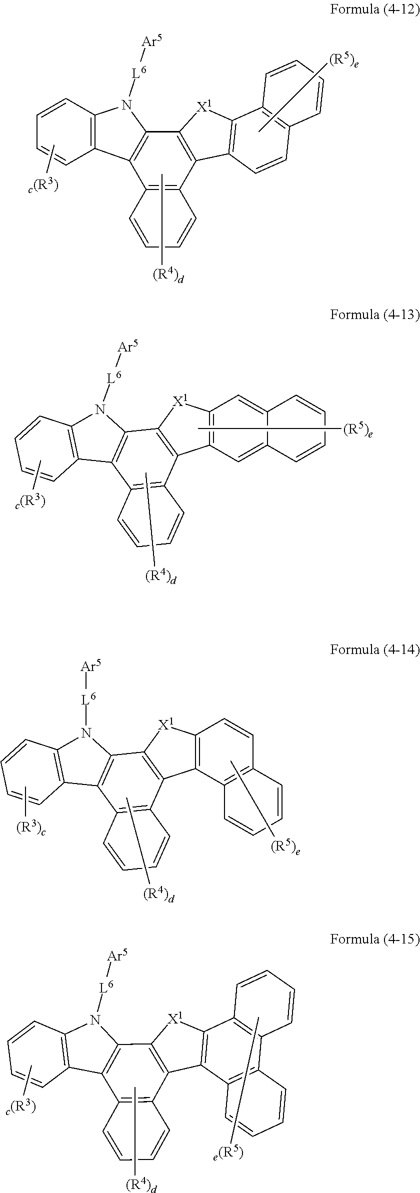

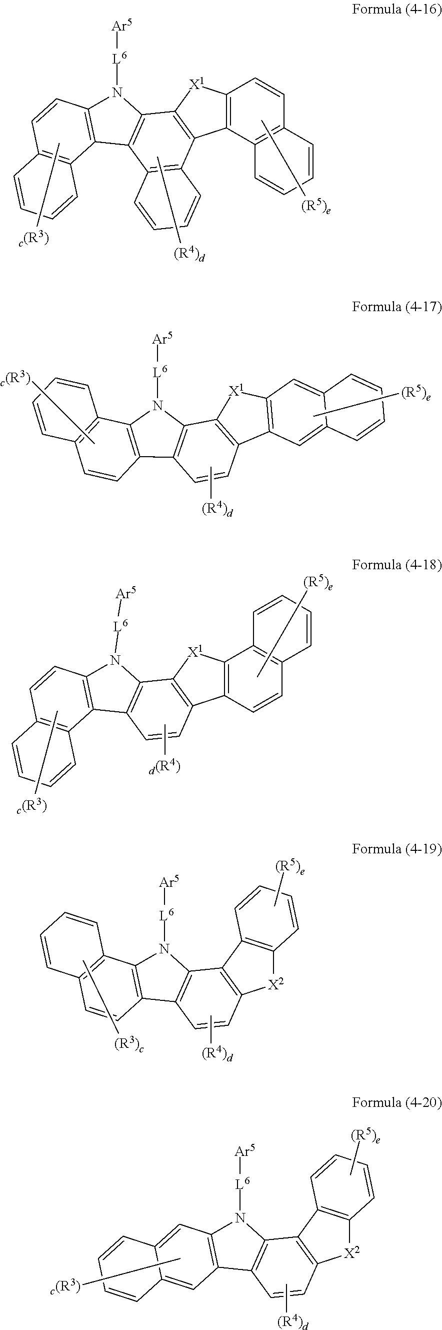

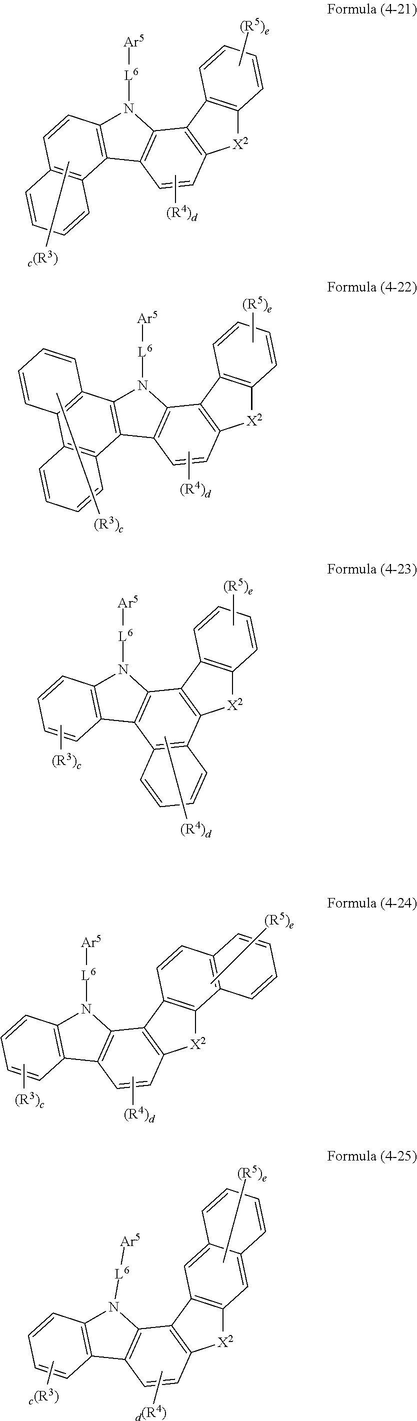

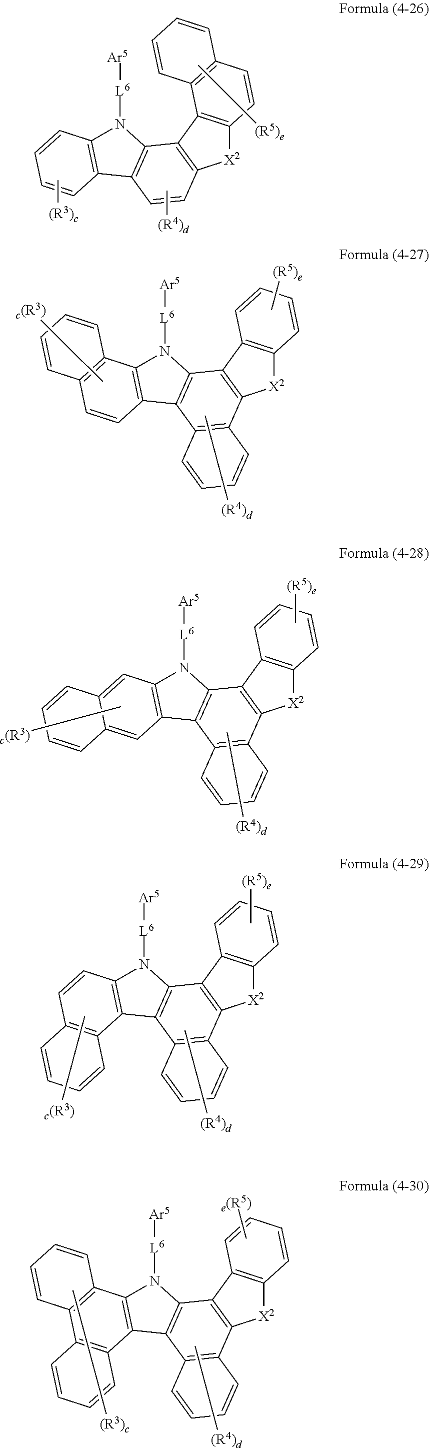

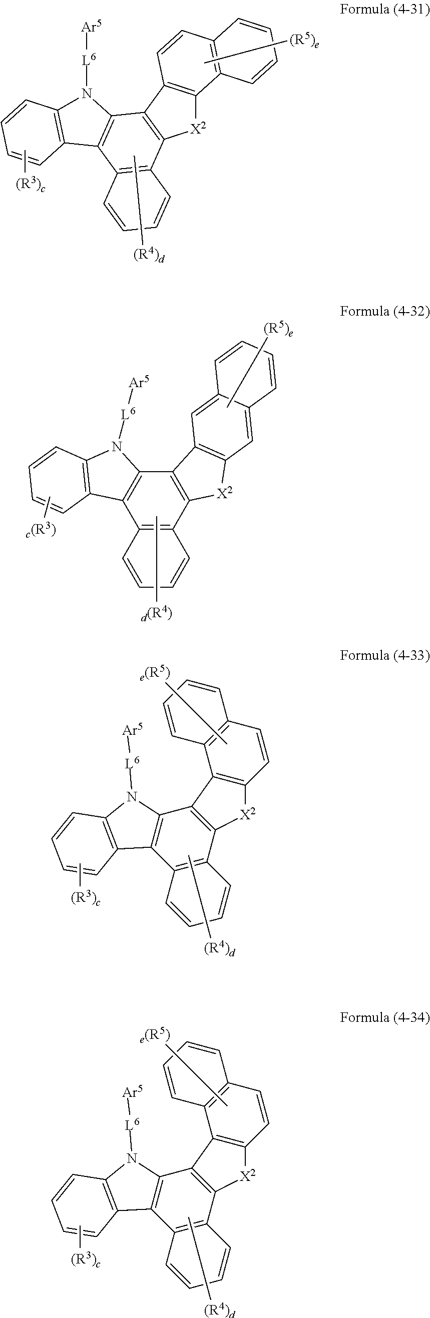

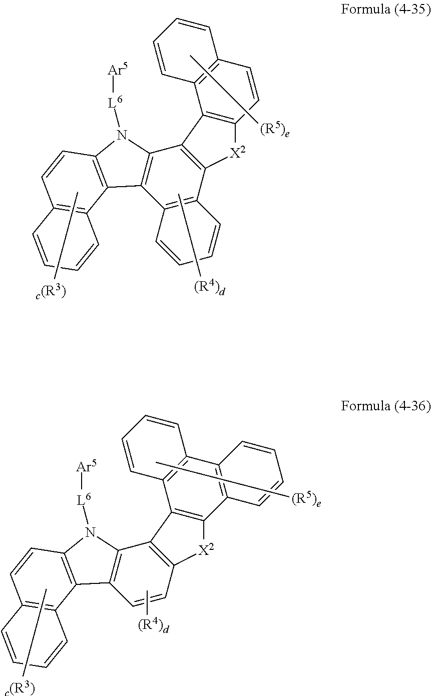

### {In Formulas (4-1) to (4-36),

Ar^{y}, L⁶, X¹, X², R³, R⁴, R⁵, c and e are the same as defined above, and d is any one of integer of 0 to 4.}

The second host compound represented by Formula (2) comprises compounds represented by the following Formulas (6-1) to (6-8).

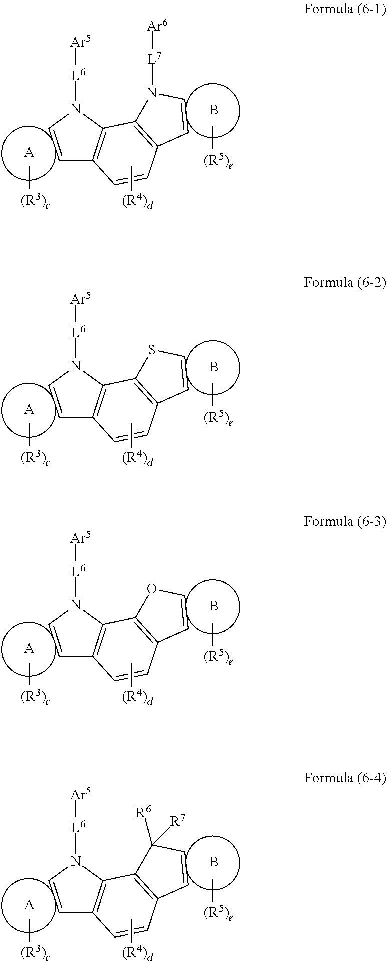

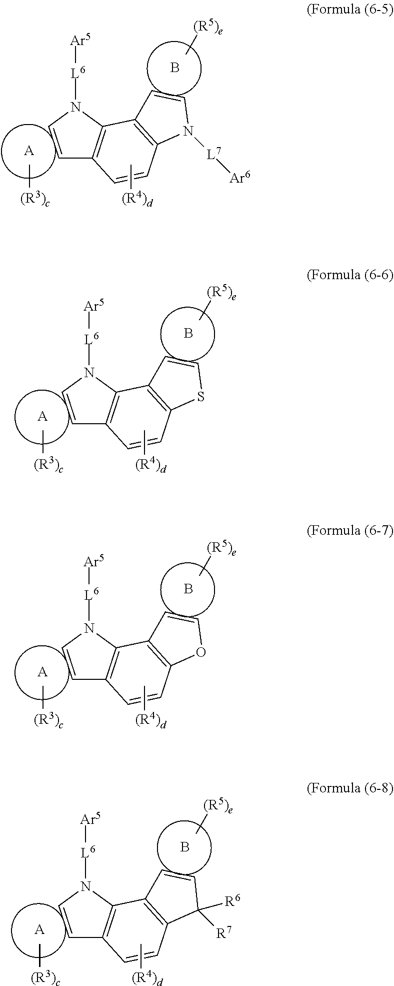

### {In Formulas (6-1) to (6-8),

R³, R⁴, R⁵, R⁶, R⁷, L⁶, L⁷, Ar⁵, Ar⁶, c, d, e, A and B are the same as defined above.}

In the above Formulas of the present invention, when Ar¹, Ar², Ar³, Ar⁴, Ar⁵, Ar⁶, Ar⁷ and R¹, R², R³, R⁴ and R⁵ are aryl groups, it is preferably C₆-C₃₀ aryl group, more preferably C₆-C₂₄ aryl group, and when Ar¹, Ar², Ar³, Ar⁴, Ar⁵, Ar⁶, Ar⁷ and R¹, R², R³, R⁴, R⁵, R⁶, R⁷, R⁸, R⁹, R¹⁰ are heterocyclic groups, it is preferably a C₂-C₄₀ heterocyclic group, more preferably a C₂-C₃₀ heterocyclic group, still more preferably a C₂-C₂₄ heterocyclic group.

when Ar¹, Ar², Ar³, Ar⁴, Ar⁵, Ar⁶, Ar⁷ and R¹, R², R³, R⁴, R⁵, R⁶, R⁷, R⁸, R⁹, R¹⁰ are aryl groups, specific examples thereof include phenyl, biphenyl, terphenyl, quaterphenyl, stylbenyl, naphthyl, anthracenyl, phenanthryl, pyrenyl, perylenyl, klycenyl group, and the like. When Ar¹, Ar², Ar³, Ar⁴, Ar⁵, Ar⁶, Ar⁷ and R¹, R², R³, R⁴, R⁵, R⁶, R⁷, R⁸, R⁹, R¹⁰ are heterocyclic groups, specific examples thereof include a thiophene group, a furan group, a pyrrole group, an imidazole group, a thiazole group, an oxazole group, an oxadiazole group, a triazole group, a pyridyl group, a bipyridyl group, a pyrimidyl group, a triazine group, a pyrazine group, a triazole group, an acridyl group, a pyridazine group, a pyrazinyl group, a quinolinyl group, a quinazolinyl group, a quinoxalinyl group, a benzoquinoxaline, a dibenzoquinoxaline, a phthalazinyl group, a pyridopyrimidinyl group, a pyridopyrazinyl group, a pyrazinopyrazinyl group, an isoquinoline group, an indole group, a carbazole group, indolocarbazole, acridine, phenoxazine, benzopyridazine, benzopyrimidine, carboline, benzocarboline, benzoxazole group, a benzoimidazole group, a benzothiazole group, a benzocarbazole group, a benzothiophene group, dibenzothiophene group, a benzofuranyl group, a phenanthroline group, a thiazolyl group, an isoxazolyl group, an oxadiazolyl group, a thiadiazolyl group, a benzothiazolyl group, a phenothiazinyl group and dibenzofuranyl group, thienothiophene, benzothienopyridine, benzothienopyrimidine, benzofuropyrimidine, dimethylbenzoindenopyrimidine, phenanthrofuropyrimidine, naphthofuropyrimidine, naphthothienopyrimidine, dibenzothiophene group, thianthrene, dihydrobenzothiophenopyrazine, dihydrobenzofuropyrazine, and the like, but are not limited thereto.

Also, when L¹, L², L³, L⁴, L⁵ and L⁶ in Formula of the present invention are an arylene group, it may preferably be an C₆-C₃₀ arylene group, more preferably an C₆-C₁₈ arylene group, illustratively, it may be phenylene, biphenyl, terphenyl, naphthalene, anthracene, phenanthrene, and the like. Preferably, L¹ is a heterocyclic group, it is preferably a C₂-C₃₀ heterocyclic group, more preferably a C₂-C₁₈ heterocyclic group, illustratively, it can be dibenzofuran, dibenzothiophene, carbazole, and the like, and when L¹ is a fluorenylene group, it can be exemplarily 9,9-dimethyl-9H-fluorene.

In the present invention, the first host compound represented by Formula (1) comprises the following Compounds 1-1 to 1-60 and 2-1 to 2-106.

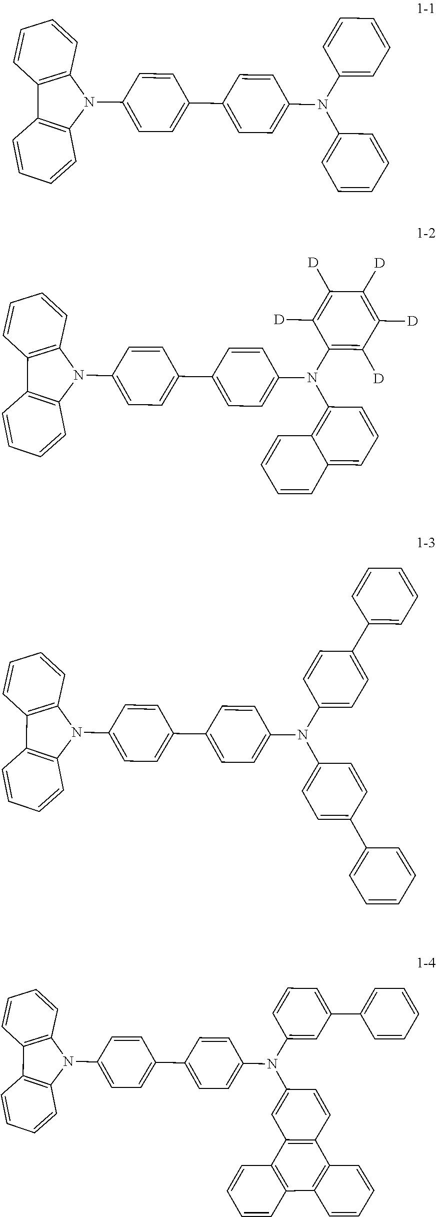 [SMILES: c1ccc(N(c2ccccc2)c2ccc(-c3ccc(-n4c5ccccc5c5ccccc54)cc3)cc2)cc1 ; [2H]c1c([2H])c([2H])c(N(c2ccc(-c3ccc(-n4c5ccccc5c5ccccc54)cc3)cc2)c2cccc3ccccc23)c([2H])c1[2H] ; c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2cccc(N(c3ccc(-c4ccc(-n5c6ccccc6c6ccccc65)cc4)cc3)c3ccc4c5ccccc5c5ccccc5c4c3)c2)cc1]

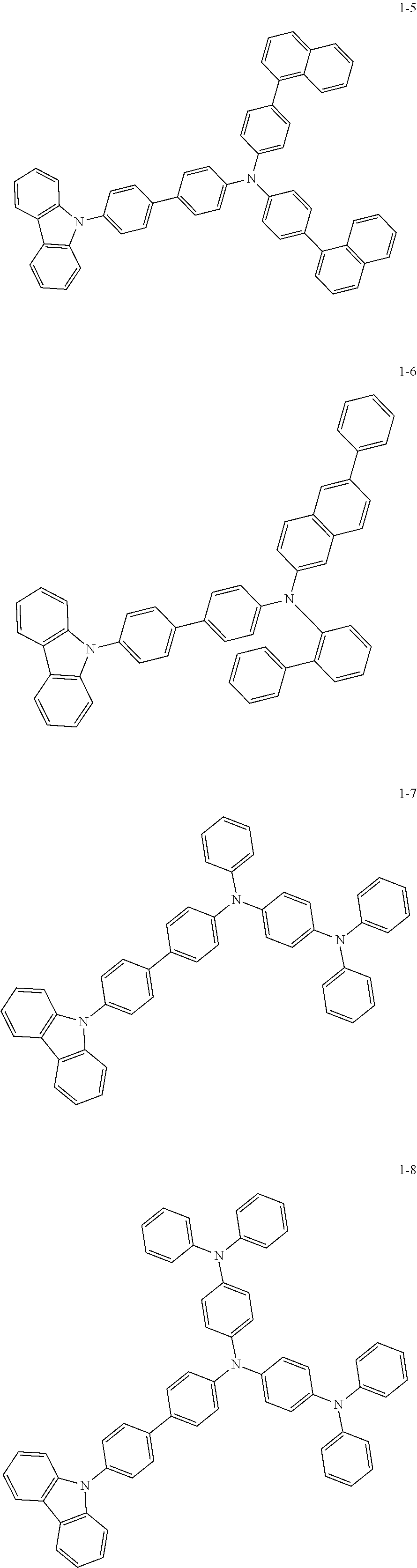 [SMILES: c1ccc2c(-c3ccc(N(c4ccc(-c5ccc(-n6c7ccccc7c7ccccc76)cc5)cc4)c4ccc(-c5cccc6ccccc56)cc4)cc3)cccc2c1 ; c1ccc(-c2ccc3cc(N(c4ccc(-c5ccc(-n6c7ccccc7c7ccccc76)cc5)cc4)c4ccccc4-c4ccccc4)ccc3c2)cc1 ; c1ccc(N(c2ccccc2)c2ccc(N(c3ccccc3)c3ccc(-c4ccc(-n5c6ccccc6c6ccccc65)cc4)cc3)cc2)cc1 ; c1ccc(N(c2ccccc2)c2ccc(N(c3ccc(-c4ccc(-n5c6ccccc6c6ccccc65)cc4)cc3)c3ccc(N(c4ccccc4)c4ccccc4)cc3)cc2)cc1]

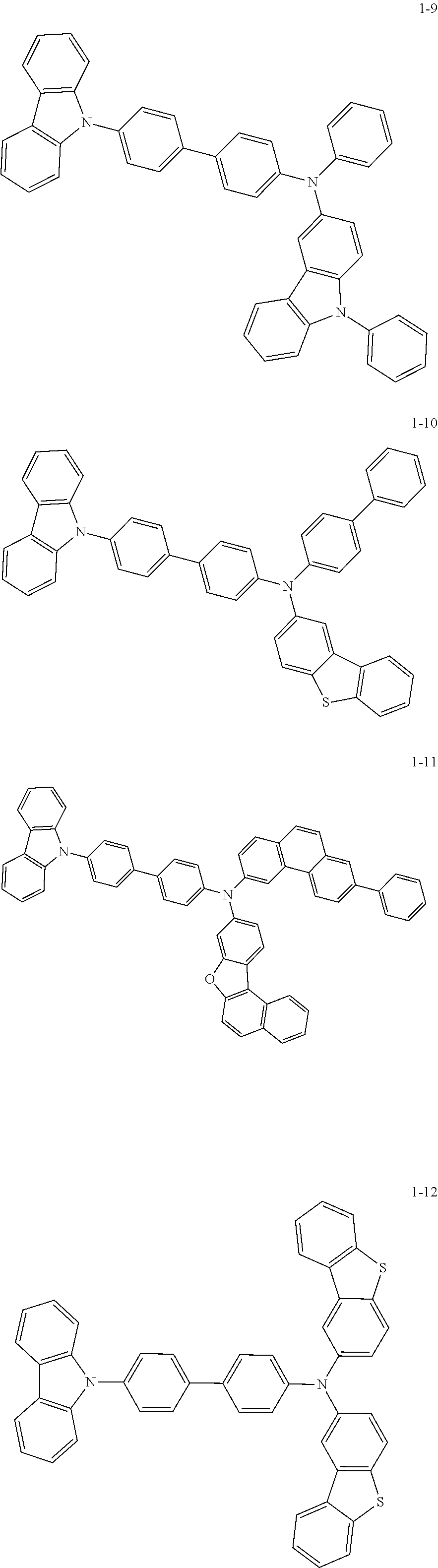 [SMILES: c1ccc(N(c2ccc(-c3ccc(-n4c5ccccc5c5ccccc54)cc3)cc2)c2ccc3c(c2)c2ccccc2n3-c2ccccc2)cc1 ; c1ccc(-c2ccc(N(c3ccc(-c4ccc(-n5c6ccccc6c6ccccc65)cc4)cc3)c3ccc4sc5ccccc5c4c3)cc2)cc1 ; c1ccc(-c2ccc3c(ccc4ccc(N(c5ccc(-c6ccc(-n7c8ccccc8c8ccccc87)cc6)cc5)c5ccc6c(c5)oc5ccc7ccccc7c56)cc43)c2)cc1 ; c1ccc2c(c1)sc1ccc(N(c3ccc(-c4ccc(-n5c6ccccc6c6ccccc65)cc4)cc3)c3ccc4sc5ccccc5c4c3)cc12]

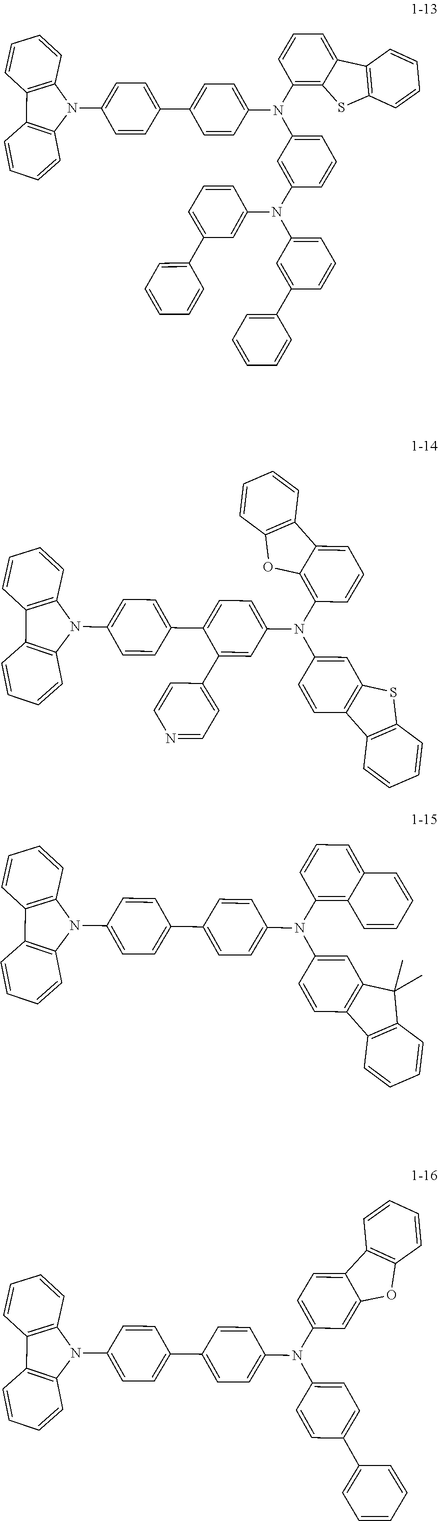 [SMILES: c1ccc(-c2cccc(N(c3cccc(-c4ccccc4)c3)c3cccc(N(c4ccc(-c5ccc(-n6c7ccccc7c7ccccc76)cc5)cc4)c4cccc5c4sc4ccccc45)c3)c2)cc1 ; c1ccc2c(c1)oc1c(N(c3ccc(-c4ccc(-n5c6ccccc6c6ccccc65)cc4)c(-c4ccncc4)c3)c3ccc4c(c3)sc3ccccc34)cccc12 ; CC1(C)c2ccccc2-c2ccc(N(c3ccc(-c4ccc(-n5c6ccccc6c6ccccc65)cc4)cc3)c3cccc4ccccc34)cc21 ; c1ccc(-c2ccc(N(c3ccc(-c4ccc(-n5c6ccccc6c6ccccc65)cc4)cc3)c3ccc4c(c3)oc3ccccc34)cc2)cc1]

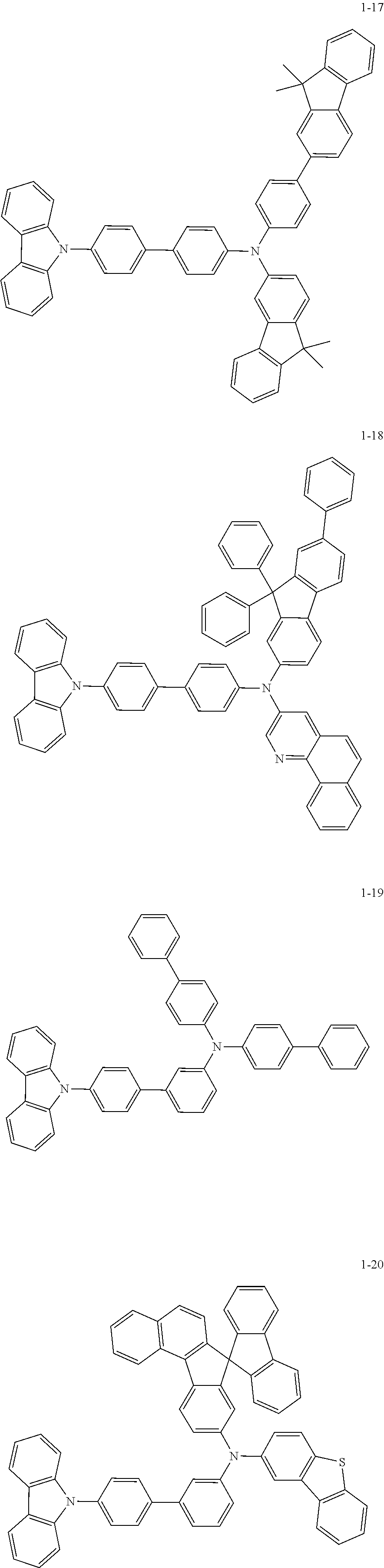 [SMILES: CC1(C)c2ccccc2-c2cc(N(c3ccc(-c4ccc(-n5c6ccccc6c6ccccc65)cc4)cc3)c3ccc(-c4ccc5c(c4)C(C)(C)c4ccccc4-5)cc3)ccc21 ; c1ccc(-c2ccc3c(c2)C(c2ccccc2)(c2ccccc2)c2cc(N(c4ccc(-c5ccc(-n6c7ccccc7c7ccccc76)cc5)cc4)c4cnc5c(ccc6ccccc65)c4)ccc2-3)cc1 ; c1ccc(-c2ccc(N(c3ccc(-c4ccccc4)cc3)c3cccc(-c4ccc(-n5c6ccccc6c6ccccc65)cc4)c3)cc2)cc1 ; c1cc(-c2ccc(-n3c4ccccc4c4ccccc43)cc2)cc(N(c2ccc3c(c2)C2(c4ccccc4-c4ccccc42)c2ccc4ccccc4c2-3)c2ccc3sc4ccccc4c3c2)c1]

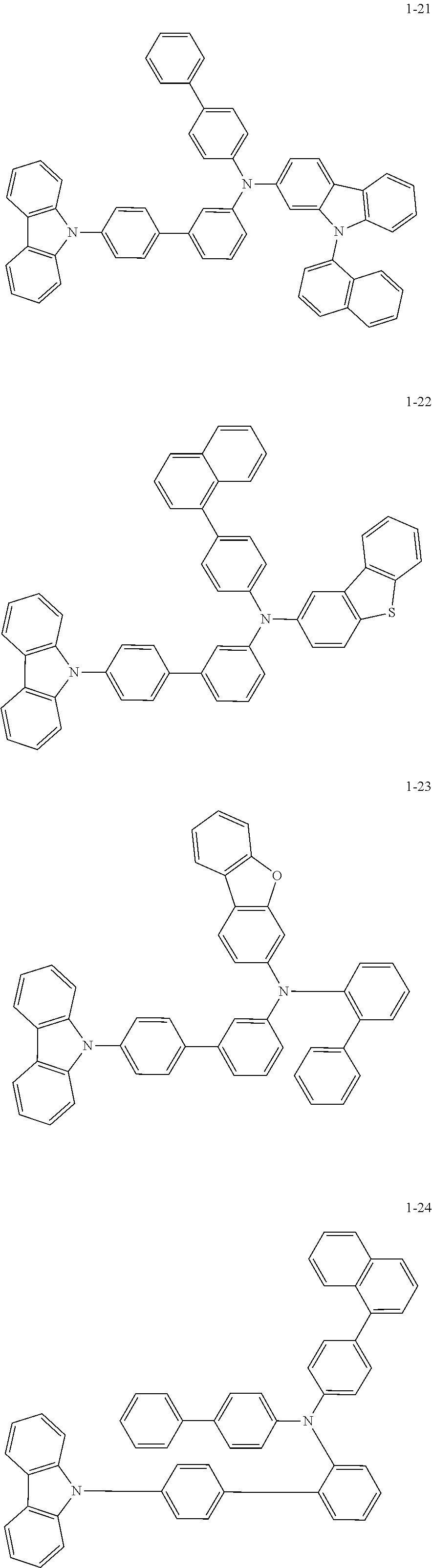 [SMILES: c1ccc(-c2ccc(N(c3cccc(-c4ccc(-n5c6ccccc6c6ccccc65)cc4)c3)c3ccc4c5ccccc5n(-c5cccc6ccccc56)c4c3)cc2)cc1 ; c1cc(-c2ccc(-n3c4ccccc4c4ccccc43)cc2)cc(N(c2ccc(-c3cccc4ccccc34)cc2)c2ccc3sc4ccccc4c3c2)c1 ; c1ccc(-c2ccccc2N(c2cccc(-c3ccc(-n4c5ccccc5c5ccccc54)cc3)c2)c2ccc3c(c2)oc2ccccc23)cc1 ; c1ccc(-c2ccc(N(c3ccc(-c4cccc5ccccc45)cc3)c3ccccc3-c3ccc(-n4c5ccccc5c5ccccc54)cc3)cc2)cc1]

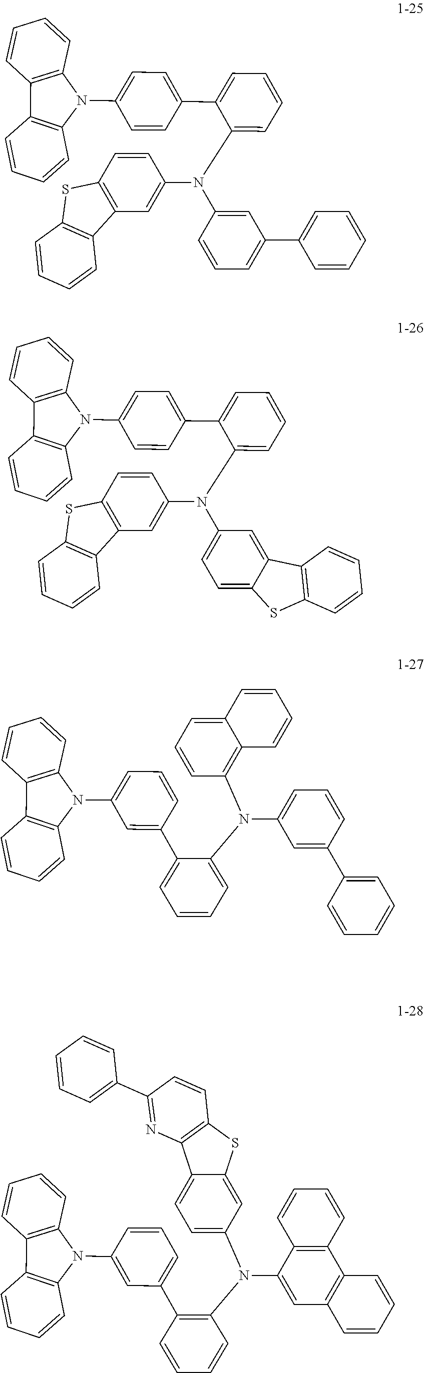 [SMILES: c1ccc(-c2cccc(N(c3ccc4sc5ccccc5c4c3)c3ccccc3-c3ccc(-n4c5ccccc5c5ccccc54)cc3)c2)cc1 ; c1ccc(N(c2ccc3sc4ccccc4c3c2)c2ccc3sc4ccccc4c3c2)c(-c2ccc(-n3c4ccccc4c4ccccc43)cc2)c1 ; c1ccc(-c2cccc(N(c3ccccc3-c3cccc(-n4c5ccccc5c5ccccc54)c3)c3cccc4ccccc34)c2)cc1 ; c1ccc(-c2ccc3sc4cc(N(c5ccccc5-c5cccc(-n6c7ccccc7c7ccccc76)c5)c5cc6ccccc6c6ccccc56)ccc4c3n2)cc1]

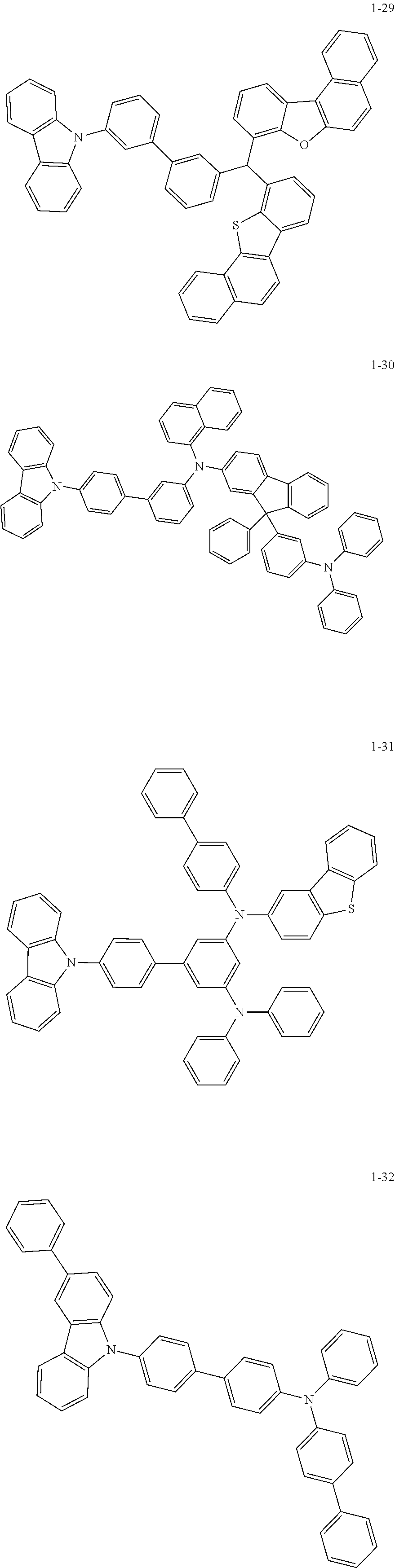 [SMILES: c1cc(-c2cccc(-n3c4ccccc4c4ccccc43)c2)cc(C(c2cccc3c2oc2ccc4ccccc4c23)c2cccc3c2sc2c4ccccc4ccc32)c1 ; c1ccc(N(c2ccccc2)c2cccc(C3(c4ccccc4)c4ccccc4-c4ccc(N(c5cccc(-c6ccc(-n7c8ccccc8c8ccccc87)cc6)c5)c5cccc6ccccc56)cc43)c2)cc1 ; c1ccc(-c2ccc(N(c3cc(-c4ccc(-n5c6ccccc6c6ccccc65)cc4)cc(N(c4ccccc4)c4ccccc4)c3)c3ccc4sc5ccccc5c4c3)cc2)cc1 ; c1ccc(-c2ccc(N(c3ccccc3)c3ccc(-c4ccc(-n5c6ccccc6c6cc(-c7ccccc7)ccc65)cc4)cc3)cc2)cc1]

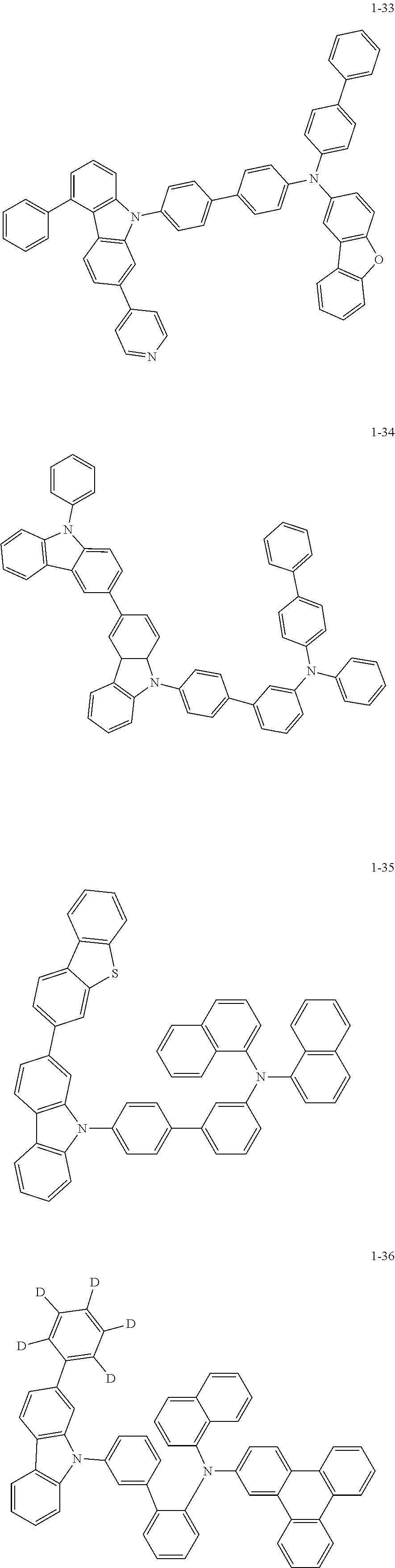 [SMILES: c1ccc(-c2ccc(N(c3ccc(-c4ccc(-n5c6cc(-c7ccncc7)ccc6c6c(-c7ccccc7)cccc65)cc4)cc3)c3ccc4oc5ccccc5c4c3)cc2)cc1 ; C1=CC2C(C=C1c1ccc3c(c1)c1ccccc1n3-c1ccccc1)c1ccccc1N2c1ccc(-c2cccc(N(c3ccccc3)c3ccc(-c4ccccc4)cc3)c2)cc1 ; c1cc(-c2ccc(-n3c4ccccc4c4ccc(-c5ccc6c(c5)sc5ccccc56)cc43)cc2)cc(N(c2cccc3ccccc23)c2cccc3ccccc23)c1 ; [2H]c1c([2H])c([2H])c(-c2ccc3c4ccccc4n(-c4cccc(-c5ccccc5N(c5ccc6c7ccccc7c7ccccc7c6c5)c5cccc6ccccc56)c4)c3c2)c([2H])c1[2H]]

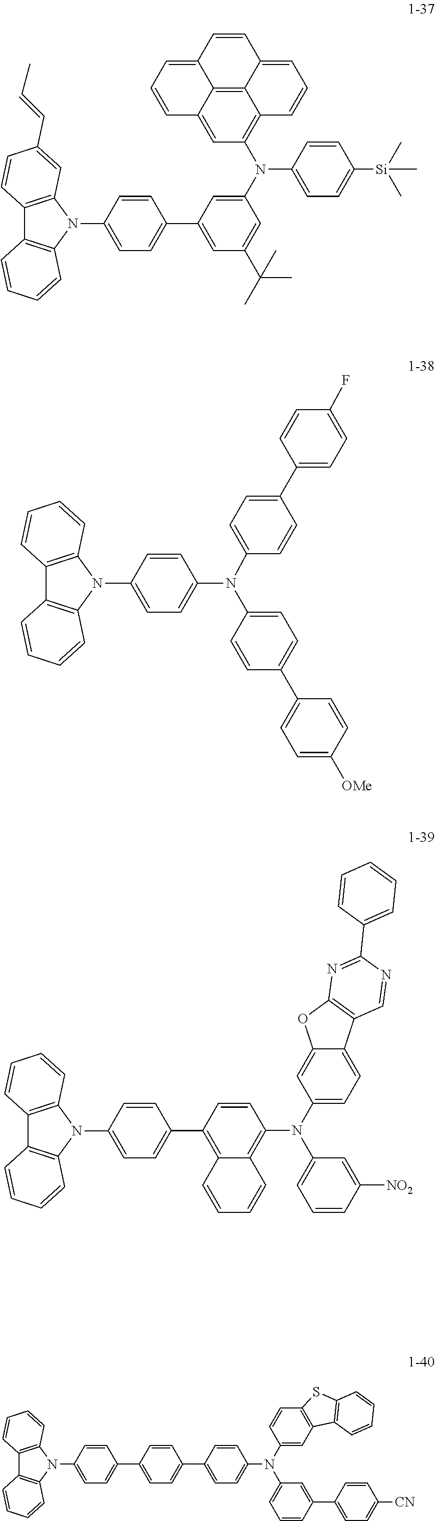 [SMILES: C/C=C/c1ccc2c3ccccc3n(-c3ccc(-c4cc(N(c5ccc([Si](C)(C)C)cc5)c5cc6cccc7ccc8cccc5c8c76)cc(C(C)(C)C)c4)cc3)c2c1 ; COc1ccc(-c2ccc(N(c3ccc(-c4ccc(F)cc4)cc3)c3ccc(-n4c5ccccc5c5ccccc54)cc3)cc2)cc1 ; O=[N+]([O-])c1cccc(N(c2ccc3c(c2)oc2nc(-c4ccccc4)ncc23)c2ccc(-c3ccc(-n4c5ccccc5c5ccccc54)cc3)c3ccccc23)c1 ; N#Cc1ccc(-c2cccc(N(c3ccc(-c4ccc(-c5ccc(-n6c7ccccc7c7ccccc76)cc5)cc4)cc3)c3ccc4sc5ccccc5c4c3)c2)cc1]

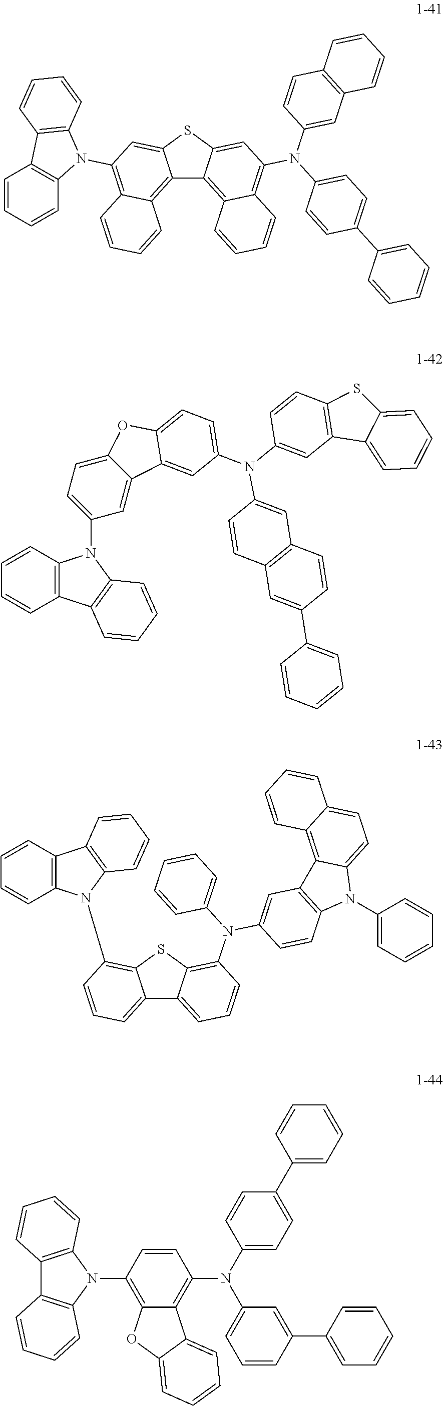 [SMILES: c1ccc(-c2ccc(N(c3ccc4ccccc4c3)c3cc4sc5cc(-n6c7ccccc7c7ccccc76)c6ccccc6c5c4c4ccccc34)cc2)cc1 ; c1ccc(-c2ccc3cc(N(c4ccc5oc6ccc(-n7c8ccccc8c8ccccc87)cc6c5c4)c4ccc5sc6ccccc6c5c4)ccc3c2)cc1 ; c1ccc(N(c2ccc3c(c2)c2c4ccccc4ccc2n3-c2ccccc2)c2cccc3c2sc2c(-n4c5ccccc5c5ccccc54)cccc23)cc1 ; c1ccc(-c2ccc(N(c3cccc(-c4ccccc4)c3)c3ccc(-n4c5ccccc5c5ccccc54)c4oc5ccccc5c34)cc2)cc1]

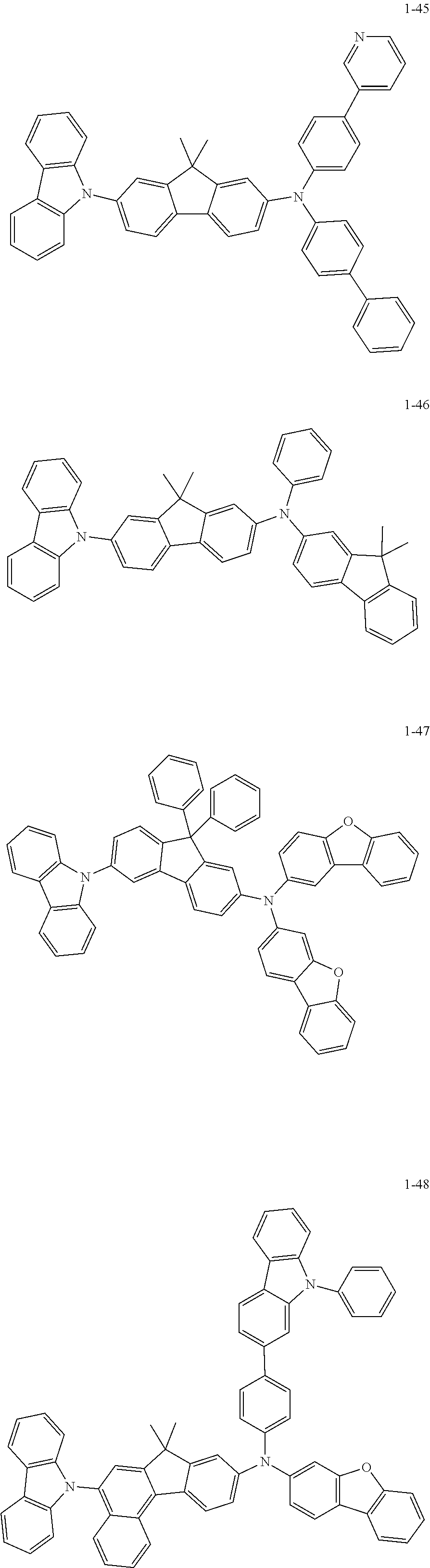 [SMILES: CC1(C)c2cc(N(c3ccc(-c4ccccc4)cc3)c3ccc(-c4cccnc4)cc3)ccc2-c2ccc(-n3c4ccccc4c4ccccc43)cc21 ; CC1(C)c2ccccc2-c2ccc(N(c3ccccc3)c3ccc4c(c3)C(C)(C)c3cc(-n5c6ccccc6c6ccccc65)ccc3-4)cc21 ; c1ccc(C2(c3ccccc3)c3ccc(-n4c5ccccc5c5ccccc54)cc3-c3ccc(N(c4ccc5c(c4)oc4ccccc45)c4ccc5oc6ccccc6c5c4)cc32)cc1 ; CC1(C)c2cc(N(c3ccc(-c4ccc5c6ccccc6n(-c6ccccc6)c5c4)cc3)c3ccc4c(c3)oc3ccccc34)ccc2-c2c1cc(-n1c3ccccc3c3ccccc31)c1ccccc21]

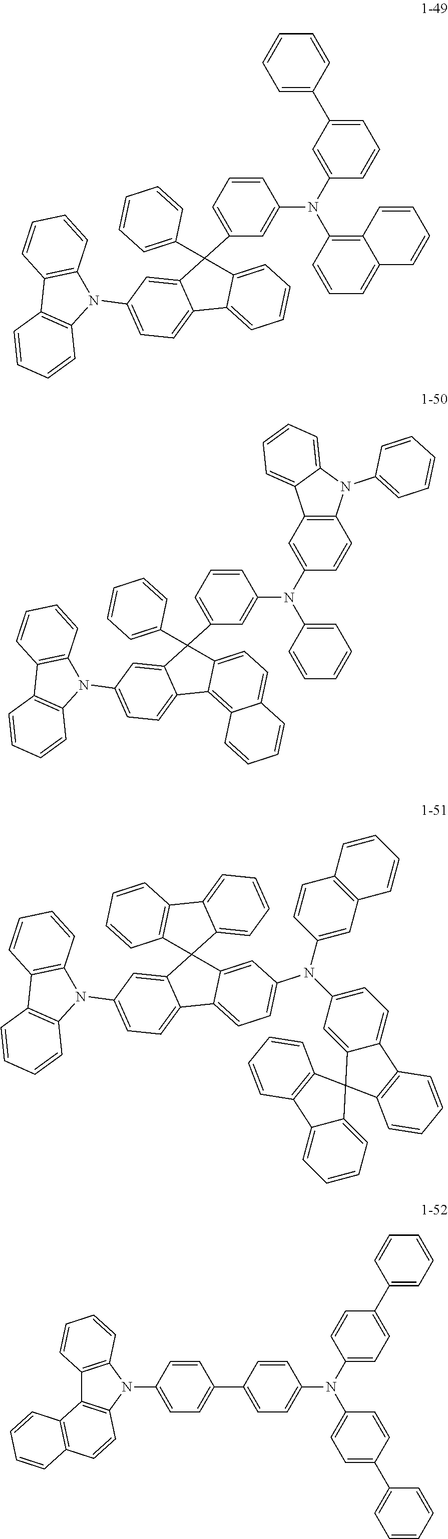 [SMILES: c1ccc(-c2cccc(N(c3cccc(C4(c5ccccc5)c5ccccc5-c5ccc(-n6c7ccccc7c7ccccc76)cc54)c3)c3cccc4ccccc34)c2)cc1 ; c1ccc(N(c2cccc(C3(c4ccccc4)c4cc(-n5c6ccccc6c6ccccc65)ccc4-c4c3ccc3ccccc43)c2)c2ccc3c(c2)c2ccccc2n3-c2ccccc2)cc1 ; c1ccc2c(c1)-c1ccccc1C21c2ccccc2-c2ccc(N(c3ccc4c(c3)C3(c5ccccc5-c5ccccc53)c3cc(-n5c6ccccc6c6ccccc65)ccc3-4)c3ccc4ccccc4c3)cc21 ; c1ccc(-c2ccc(N(c3ccc(-c4ccccc4)cc3)c3ccc(-c4ccc(-n5c6ccccc6c6c7ccccc7ccc65)cc4)cc3)cc2)cc1]

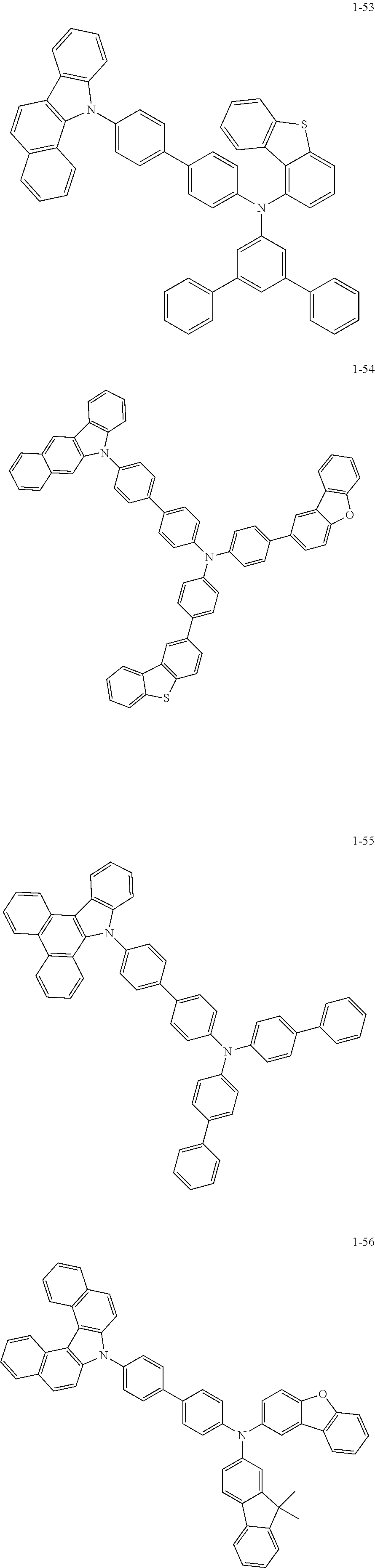 [SMILES: c1ccc(-c2cc(-c3ccccc3)cc(N(c3ccc(-c4ccc(-n5c6ccccc6c6ccc7ccccc7c65)cc4)cc3)c3cccc4sc5ccccc5c34)c2)cc1 ; c1ccc2cc3c(cc2c1)c1ccccc1n3-c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4ccccc4)cc3)c3ccc(-c4ccc(-n5c6ccccc6c6c7ccccc7c7ccccc7c65)cc4)cc3)cc2)cc1 ; CC1(C)c2ccccc2-c2ccc(N(c3ccc(-c4ccc(-n5c6ccc7ccccc7c6c6c7ccccc7ccc65)cc4)cc3)c3ccc4oc5ccccc5c4c3)cc21]

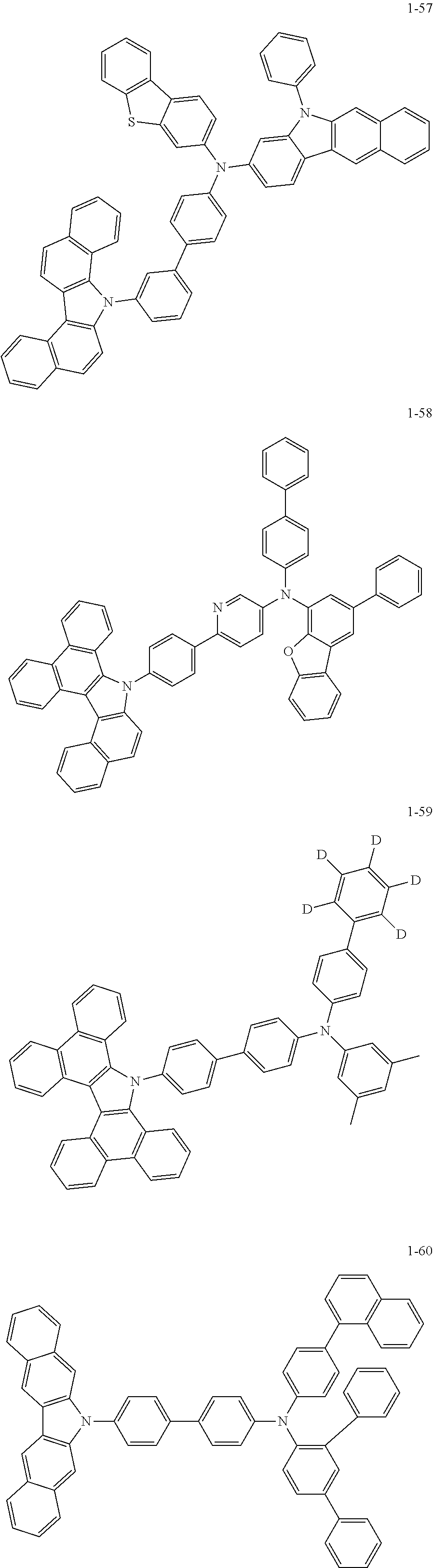 [SMILES: c1ccc(-n2c3cc(N(c4ccc(-c5cccc(-n6c7ccc8ccccc8c7c7ccc8ccccc8c76)c5)cc4)c4ccc5c(c4)sc4ccccc45)ccc3c3cc4ccccc4cc32)cc1 ; c1ccc(-c2ccc(N(c3ccc(-c4ccc(-n5c6ccc7ccccc7c6c6c7ccccc7c7ccccc7c65)cc4)nc3)c3cc(-c4ccccc4)cc4c3oc3ccccc34)cc2)cc1 ; [2H]c1c([2H])c([2H])c(-c2ccc(N(c3ccc(-c4ccc(-n5c6c7ccccc7c7ccccc7c6c6c7ccccc7c7ccccc7c65)cc4)cc3)c3cc(C)cc(C)c3)cc2)c([2H])c1[2H] ; c1ccc(-c2ccc(N(c3ccc(-c4ccc(-n5c6cc7ccccc7cc6c6cc7ccccc7cc65)cc4)cc3)c3ccc(-c4cccc5ccccc45)cc3)c(-c3ccccc3)c2)cc1]

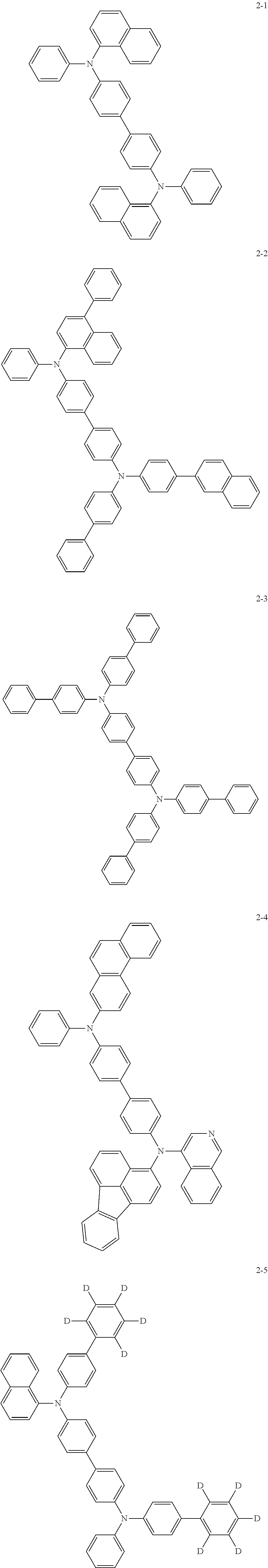

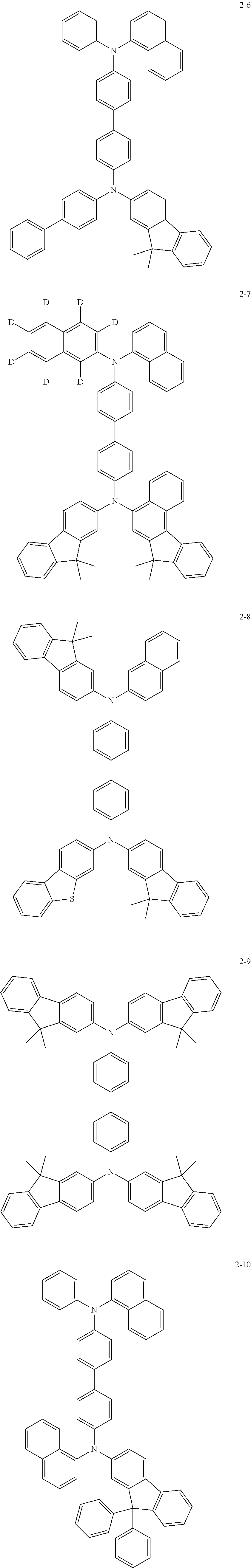

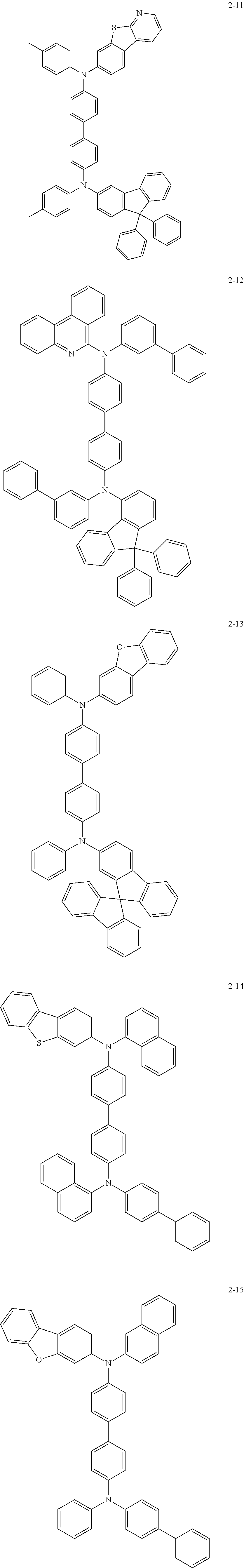

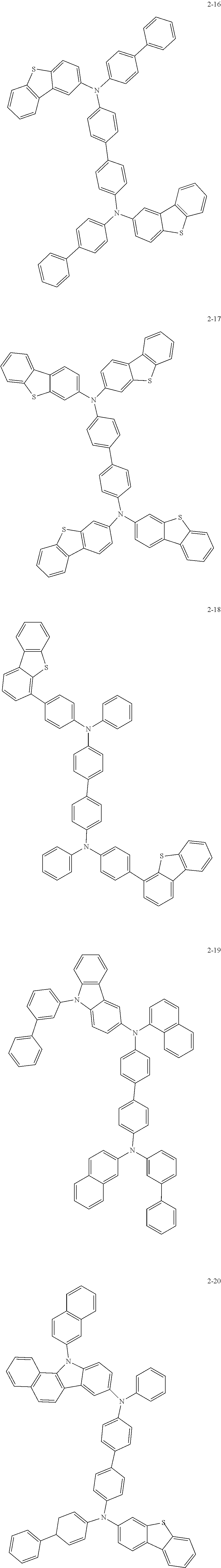

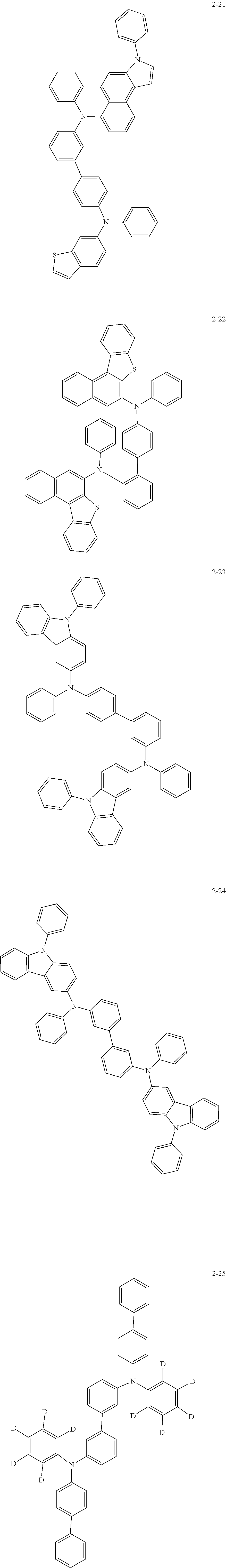

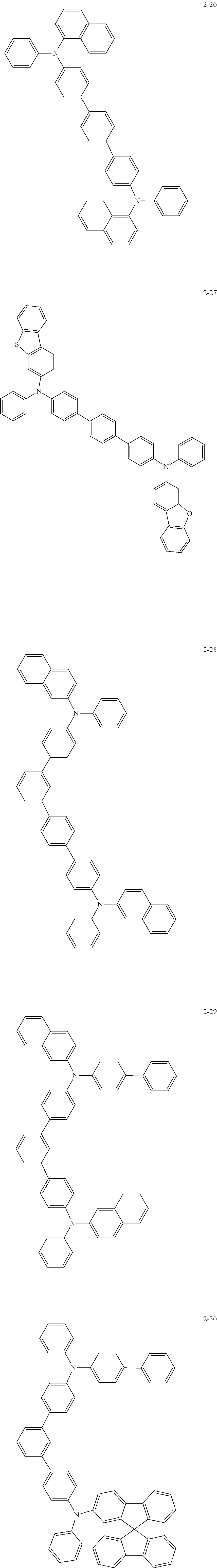

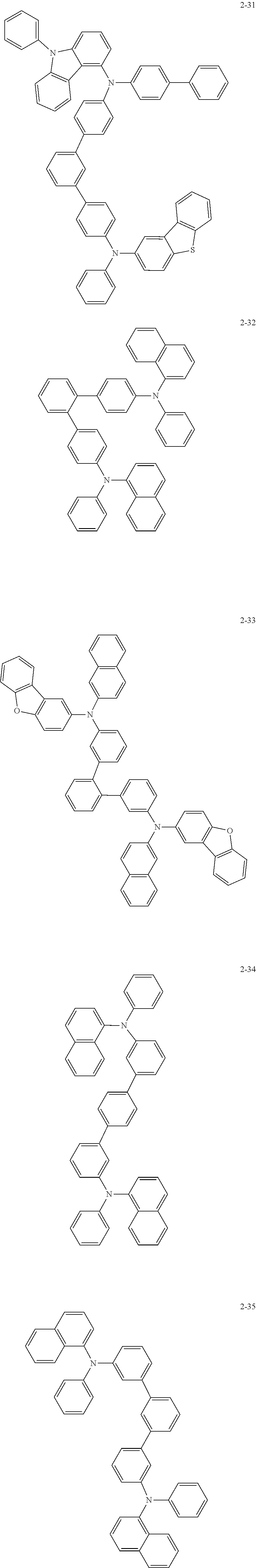 [SMILES: c1ccc(-c2ccc(N(c3ccc(-c4cccc(-c5ccc(N(c6ccccc6)c6ccc7sc8ccccc8c7c6)cc5)c4)cc3)c3cccc4c3c3ccccc3n4-c3ccccc3)cc2)cc1 ; c1ccc(N(c2ccc(-c3ccccc3-c3ccc(N(c4ccccc4)c4cccc5ccccc45)cc3)cc2)c2cccc3ccccc23)cc1 ; c1cc(-c2ccccc2-c2cccc(N(c3ccc4ccccc4c3)c3ccc4oc5ccccc5c4c3)c2)cc(N(c2ccc3ccccc3c2)c2ccc3oc4ccccc4c3c2)c1 ; c1ccc(N(c2cccc(-c3ccc(-c4cccc(N(c5ccccc5)c5cccc6ccccc56)c4)cc3)c2)c2cccc3ccccc23)cc1 ; c1ccc(N(c2cccc(-c3cccc(-c4cccc(N(c5ccccc5)c5cccc6ccccc56)c4)c3)c2)c2cccc3ccccc23)cc1]

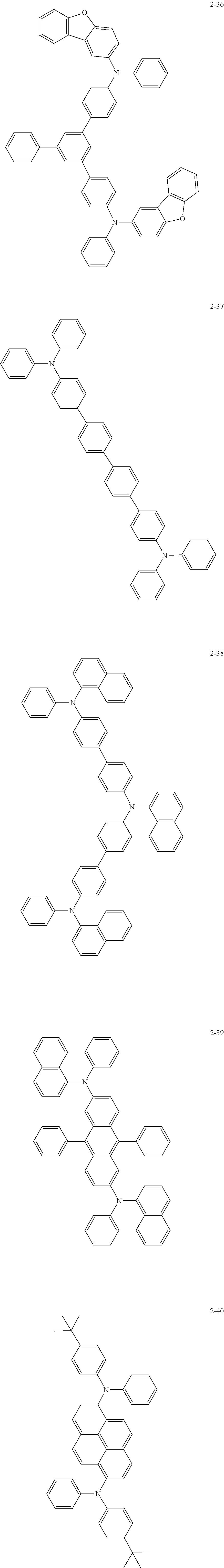 [SMILES: c1ccc(-c2cc(-c3ccc(N(c4ccccc4)c4ccc5oc6ccccc6c5c4)cc3)cc(-c3ccc(N(c4ccccc4)c4ccc5oc6ccccc6c5c4)cc3)c2)cc1 ; c1ccc(N(c2ccccc2)c2ccc(-c3ccc(-c4ccc(-c5ccc(N(c6ccccc6)c6ccccc6)cc5)cc4)cc3)cc2)cc1 ; c1ccc(N(c2ccc(-c3ccc(N(c4ccc(-c5ccc(N(c6ccccc6)c6cccc7ccccc67)cc5)cc4)c4cccc5ccccc45)cc3)cc2)c2cccc3ccccc23)cc1 ; c1ccc(-c2c3ccc(N(c4ccccc4)c4cccc5ccccc45)cc3c(-c3ccccc3)c3ccc(N(c4ccccc4)c4cccc5ccccc45)cc23)cc1 ; CC(C)(C)c1ccc(N(c2ccccc2)c2ccc3ccc4c(N(c5ccccc5)c5ccc(C(C)(C)C)cc5)ccc5ccc2c3c54)cc1]

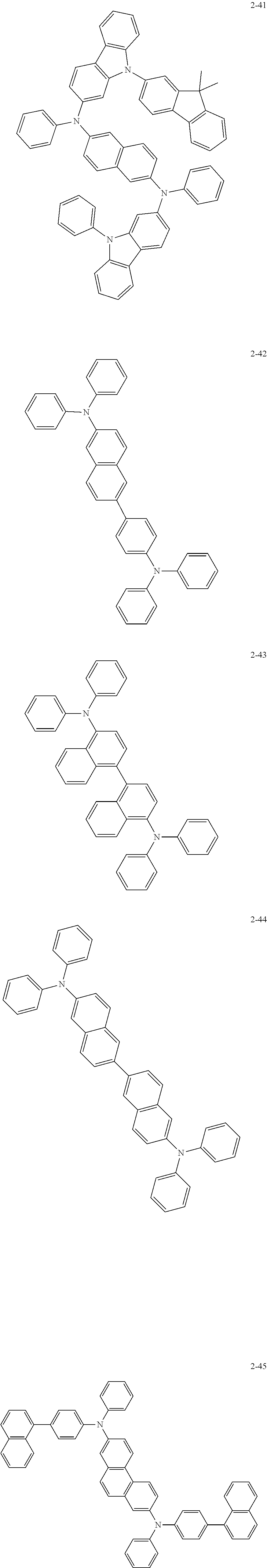 [SMILES: CC1(C)c2ccccc2-c2ccc(-n3c4ccccc4c4ccc(N(c5ccccc5)c5ccc6cc(N(c7ccccc7)c7ccc8c9ccccc9n(-c9ccccc9)c8c7)ccc6c5)cc43)cc21 ; c1ccc(N(c2ccccc2)c2ccc(-c3ccc4cc(N(c5ccccc5)c5ccccc5)ccc4c3)cc2)cc1 ; c1ccc(N(c2ccccc2)c2ccc(-c3ccc(N(c4ccccc4)c4ccccc4)c4ccccc34)c3ccccc23)cc1 ; c1ccc(N(c2ccccc2)c2ccc3cc(-c4ccc5cc(N(c6ccccc6)c6ccccc6)ccc5c4)ccc3c2)cc1 ; c1ccc(N(c2ccc(-c3cccc4ccccc34)cc2)c2ccc3c(ccc4cc(N(c5ccccc5)c5ccc(-c6cccc7ccccc67)cc5)ccc43)c2)cc1]

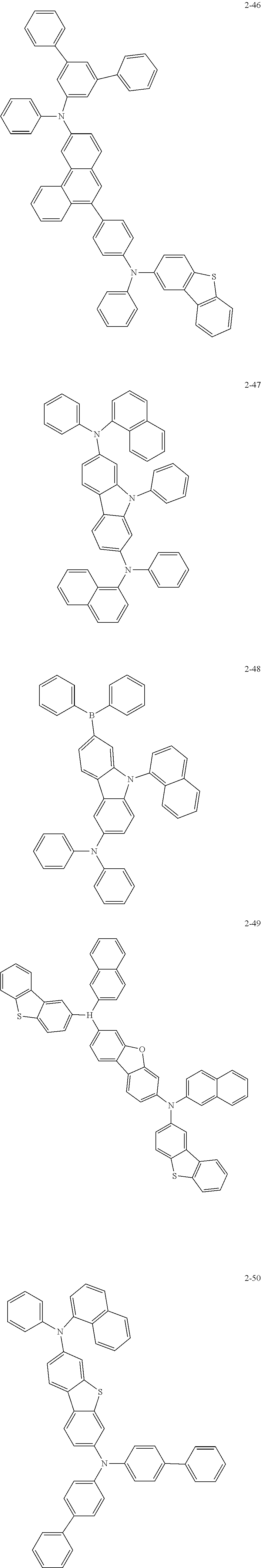

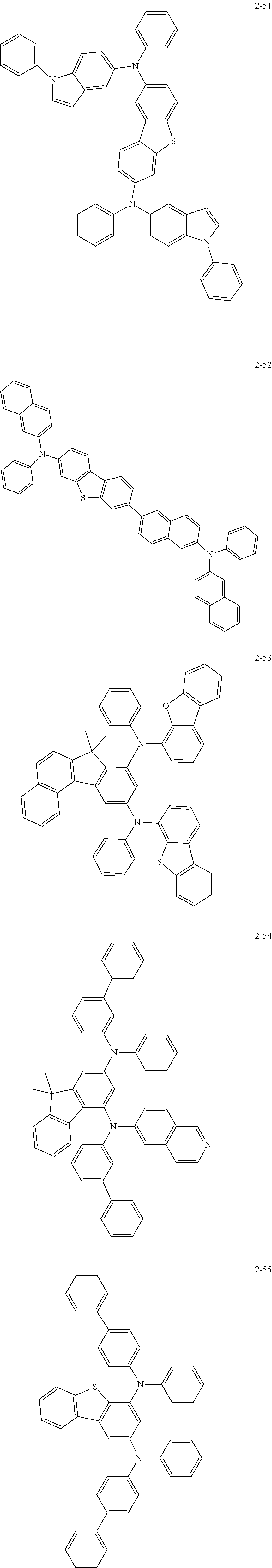 [SMILES: c1ccc(N(c2ccc3c(ccn3-c3ccccc3)c2)c2ccc3c(c2)sc2ccc(N(c4ccccc4)c4ccc5c(ccn5-c5ccccc5)c4)cc23)cc1 ; c1ccc(N(c2ccc3ccccc3c2)c2ccc3cc(-c4ccc5c(c4)sc4cc(N(c6ccccc6)c6ccc7ccccc7c6)ccc45)ccc3c2)cc1 ; CC1(C)c2ccc3ccccc3c2-c2cc(N(c3ccccc3)c3cccc4c3sc3ccccc34)cc(N(c3ccccc3)c3cccc4c3oc3ccccc34)c21 ; CC1(C)c2ccccc2-c2c(N(c3cccc(-c4ccccc4)c3)c3ccc4cnccc4c3)cc(N(c3ccccc3)c3cccc(-c4ccccc4)c3)cc21 ; c1ccc(-c2ccc(N(c3ccccc3)c3cc(N(c4ccccc4)c4ccc(-c5ccccc5)cc4)c4sc5ccccc5c4c3)cc2)cc1]

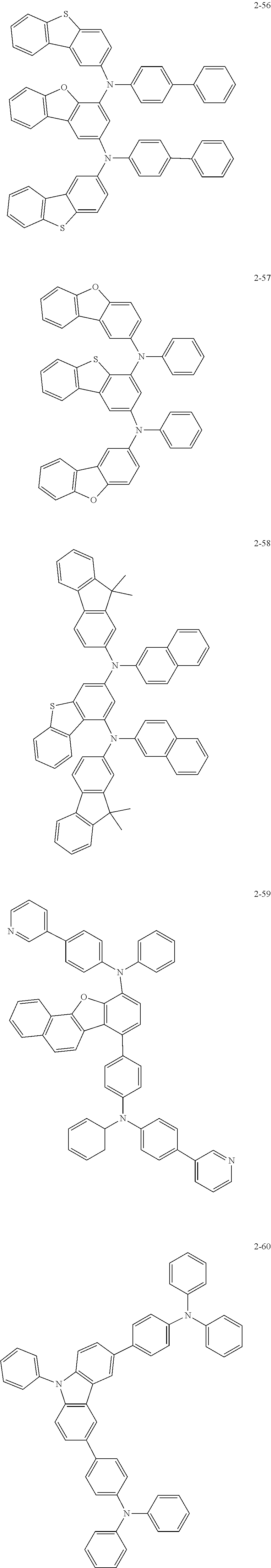

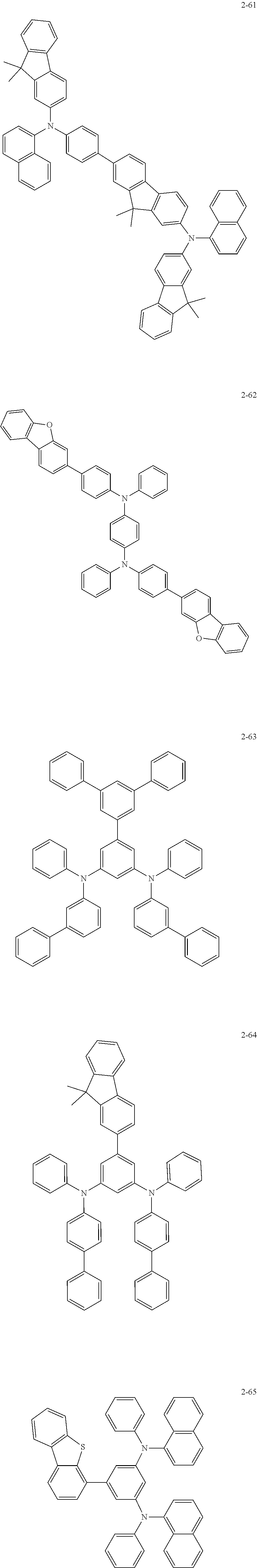

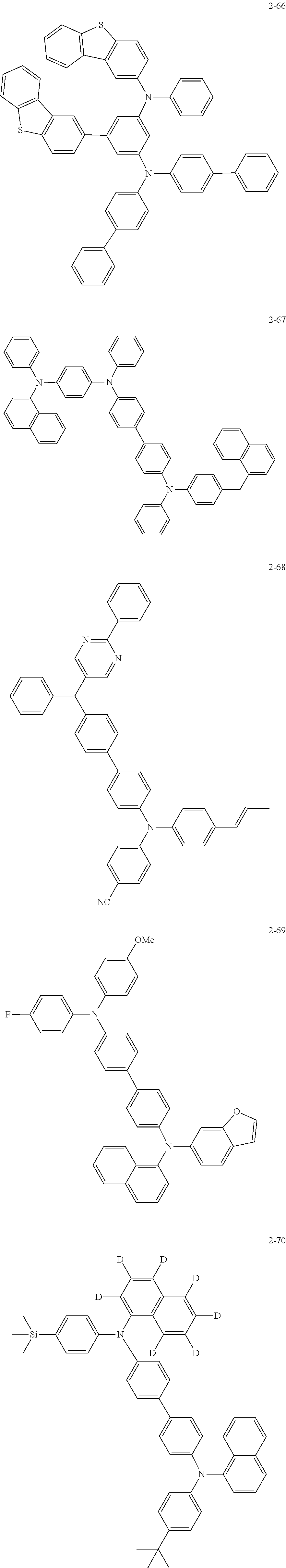

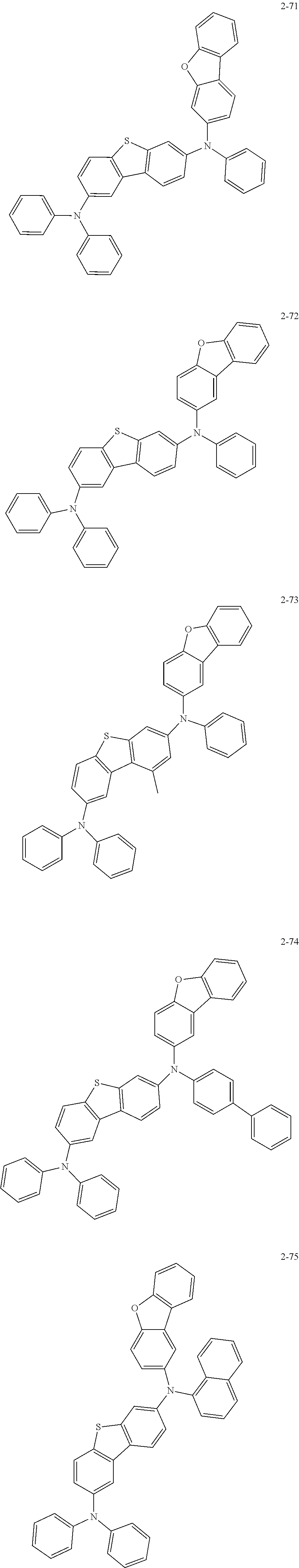 [SMILES: c1ccc(N(c2ccc3c(c2)oc2ccccc23)c2ccc3c(c2)sc2ccc(N(c4ccccc4)c4ccccc4)cc23)cc1 ; c1ccc(N(c2ccc3c(c2)sc2ccc(N(c4ccccc4)c4ccccc4)cc23)c2ccc3oc4ccccc4c3c2)cc1 ; Cc1cc(N(c2ccccc2)c2ccc3oc4ccccc4c3c2)cc2sc3ccc(N(c4ccccc4)c4ccccc4)cc3c12 ; c1ccc(-c2ccc(N(c3ccc4c(c3)sc3ccc(N(c5ccccc5)c5ccccc5)cc34)c3ccc4oc5ccccc5c4c3)cc2)cc1 ; c1ccc(N(c2ccccc2)c2ccc3sc4cc(N(c5ccc6oc7ccccc7c6c5)c5cccc6ccccc56)ccc4c3c2)cc1]

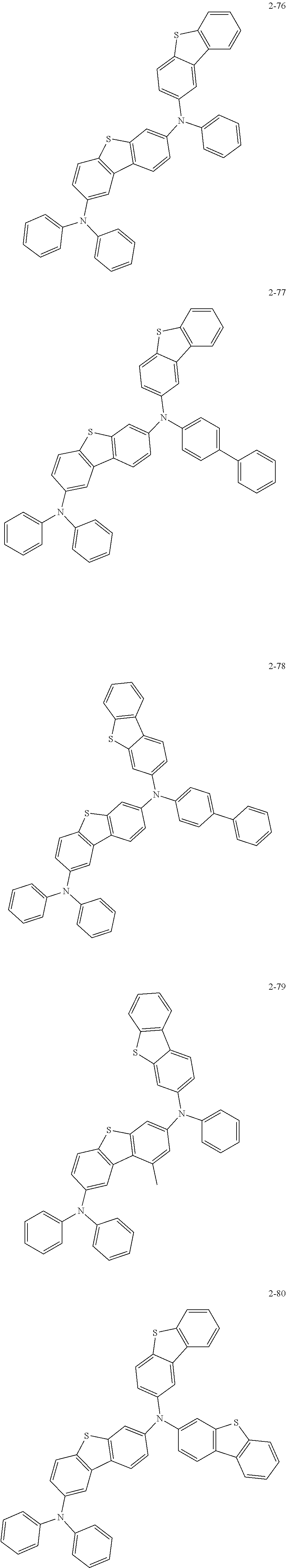 [SMILES: c1ccc(N(c2ccc3c(c2)sc2ccc(N(c4ccccc4)c4ccccc4)cc23)c2ccc3sc4ccccc4c3c2)cc1 ; c1ccc(-c2ccc(N(c3ccc4c(c3)sc3ccc(N(c5ccccc5)c5ccccc5)cc34)c3ccc4sc5ccccc5c4c3)cc2)cc1 ; c1ccc(-c2ccc(N(c3ccc4c(c3)sc3ccccc34)c3ccc4c(c3)sc3ccc(N(c5ccccc5)c5ccccc5)cc34)cc2)cc1 ; Cc1cc(N(c2ccccc2)c2ccc3c(c2)sc2ccccc23)cc2sc3ccc(N(c4ccccc4)c4ccccc4)cc3c12 ; c1ccc(N(c2ccccc2)c2ccc3sc4cc(N(c5ccc6c(c5)sc5ccccc56)c5ccc6sc7ccccc7c6c5)ccc4c3c2)cc1]

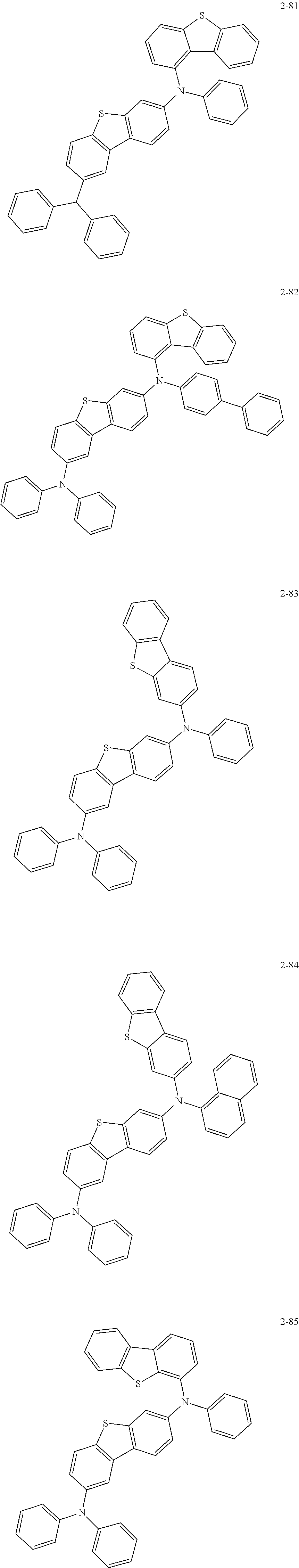 [SMILES: c1ccc(C(c2ccccc2)c2ccc3sc4cc(N(c5ccccc5)c5cccc6sc7ccccc7c56)ccc4c3c2)cc1 ; c1ccc(-c2ccc(N(c3ccc4c(c3)sc3ccc(N(c5ccccc5)c5ccccc5)cc34)c3cccc4sc5ccccc5c34)cc2)cc1 ; c1ccc(N(c2ccc3c(c2)sc2ccccc23)c2ccc3c(c2)sc2ccc(N(c4ccccc4)c4ccccc4)cc23)cc1 ; c1ccc(N(c2ccccc2)c2ccc3sc4cc(N(c5ccc6c(c5)sc5ccccc56)c5cccc6ccccc56)ccc4c3c2)cc1 ; c1ccc(N(c2ccccc2)c2ccc3sc4cc(N(c5ccccc5)c5cccc6c5sc5ccccc56)ccc4c3c2)cc1]

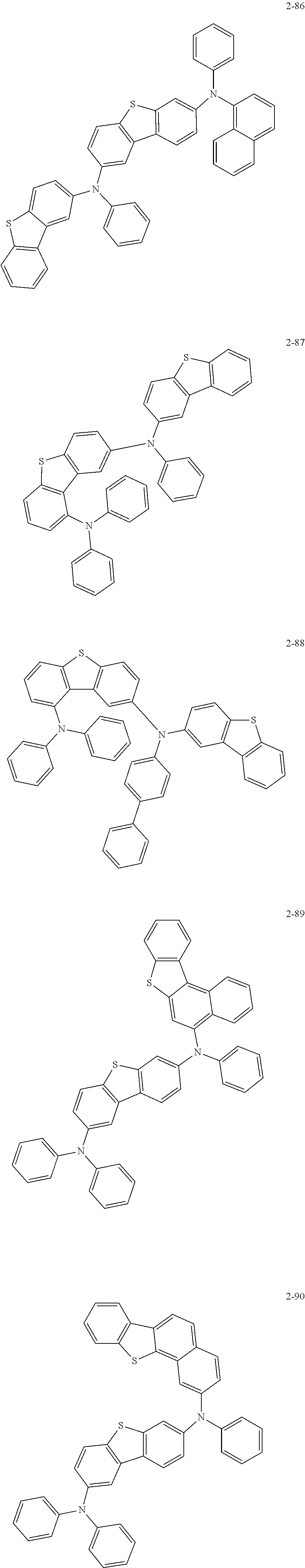 [SMILES: c1ccc(N(c2ccc3sc4ccccc4c3c2)c2ccc3sc4cc(N(c5ccccc5)c5cccc6ccccc56)ccc4c3c2)cc1 ; c1ccc(N(c2ccc3sc4ccccc4c3c2)c2ccc3sc4cccc(N(c5ccccc5)c5ccccc5)c4c3c2)cc1 ; c1ccc(-c2ccc(N(c3ccc4sc5ccccc5c4c3)c3ccc4sc5cccc(N(c6ccccc6)c6ccccc6)c5c4c3)cc2)cc1 ; c1ccc(N(c2ccccc2)c2ccc3sc4cc(N(c5ccccc5)c5cc6sc7ccccc7c6c6ccccc56)ccc4c3c2)cc1 ; c1ccc(N(c2ccccc2)c2ccc3sc4cc(N(c5ccccc5)c5ccc6ccc7c8ccccc8sc7c6c5)ccc4c3c2)cc1]

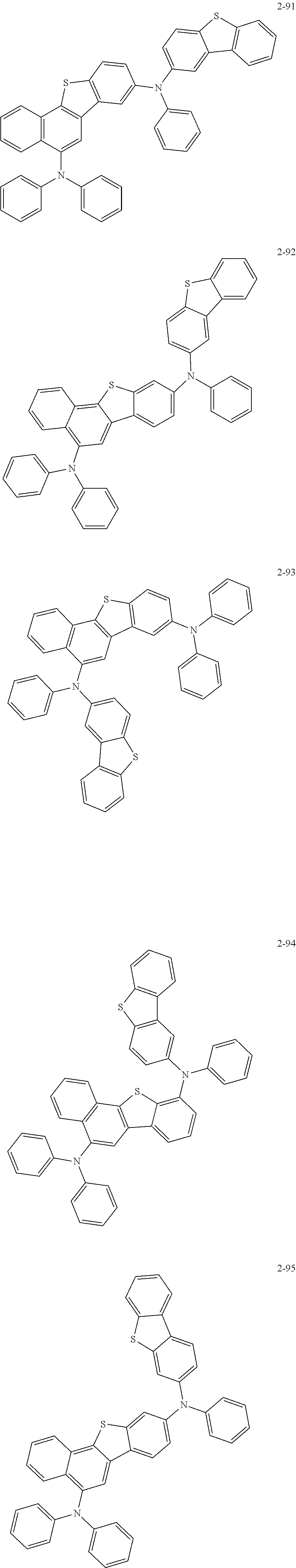 [SMILES: c1ccc(N(c2ccc3sc4ccccc4c3c2)c2ccc3sc4c5ccccc5c(N(c5ccccc5)c5ccccc5)cc4c3c2)cc1 ; c1ccc(N(c2ccc3c(c2)sc2c4ccccc4c(N(c4ccccc4)c4ccccc4)cc32)c2ccc3sc4ccccc4c3c2)cc1 ; c1ccc(N(c2ccccc2)c2ccc3sc4c5ccccc5c(N(c5ccccc5)c5ccc6sc7ccccc7c6c5)cc4c3c2)cc1 ; c1ccc(N(c2ccccc2)c2cc3c4cccc(N(c5ccccc5)c5ccc6sc7ccccc7c6c5)c4sc3c3ccccc23)cc1 ; c1ccc(N(c2ccc3c(c2)sc2ccccc23)c2ccc3c(c2)sc2c4ccccc4c(N(c4ccccc4)c4ccccc4)cc32)cc1]

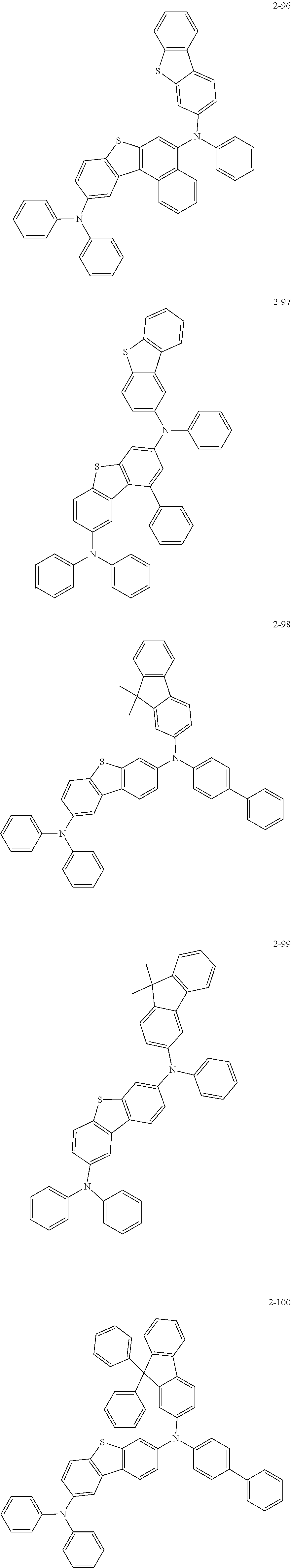 [SMILES: c1ccc(N(c2ccccc2)c2ccc3sc4cc(N(c5ccccc5)c5ccc6c(c5)sc5ccccc56)c5ccccc5c4c3c2)cc1 ; Cc1ccc(N(c2ccccc2)c2ccc3sc4cc(N(c5ccccc5)c5ccc6sc7ccccc7c6c5)cc(-c5ccccc5)c4c3c2)cc1 ; CC1(C)c2ccccc2-c2ccc(N(c3ccc(-c4ccccc4)cc3)c3ccc4c(c3)sc3ccc(N(c5ccccc5)c5ccccc5)cc34)cc21 ; CC1(C)c2ccccc2-c2cc(N(c3ccccc3)c3ccc4c(c3)sc3ccc(N(c5ccccc5)c5ccccc5)cc34)ccc21 ; c1ccc(-c2ccc(N(c3ccc4c(c3)C(c3ccccc3)(c3ccccc3)c3ccccc3-4)c3ccc4c(c3)sc3ccc(N(c5ccccc5)c5ccccc5)cc34)cc2)cc1]

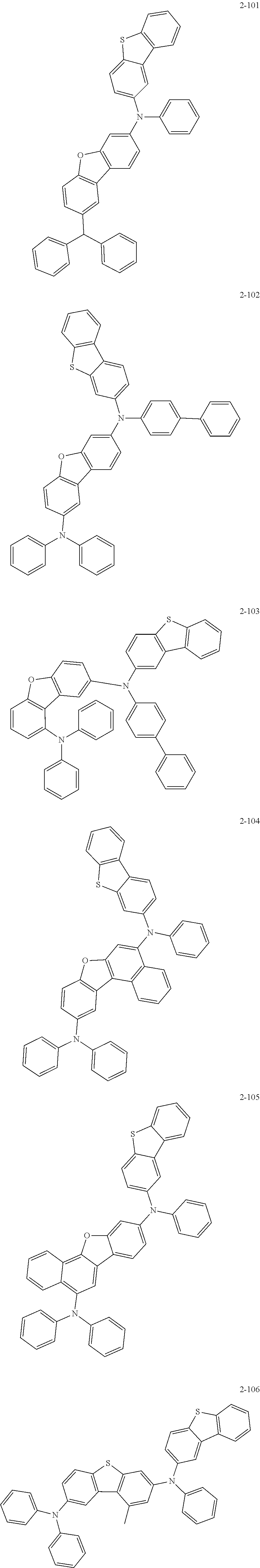 [SMILES: c1ccc(C(c2ccccc2)c2ccc3oc4cc(N(c5ccccc5)c5ccc6sc7ccccc7c6c5)ccc4c3c2)cc1 ; c1ccc(-c2ccc(N(c3ccc4c(c3)oc3ccc(N(c5ccccc5)c5ccccc5)cc34)c3ccc4c(c3)sc3ccccc34)cc2)cc1 ; c1ccc(-c2ccc(N(c3ccc4sc5ccccc5c4c3)c3ccc4oc5cccc(N(c6ccccc6)c6ccccc6)c5c4c3)cc2)cc1 ; c1ccc(N(c2ccccc2)c2ccc3oc4cc(N(c5ccccc5)c5ccc6c(c5)sc5ccccc56)c5ccccc5c4c3c2)cc1 ; c1ccc(N(c2ccc3c(c2)oc2c4ccccc4c(N(c4ccccc4)c4ccccc4)cc32)c2ccc3sc4ccccc4c3c2)cc1 ; Cc1cc(N(c2ccccc2)c2ccc3sc4ccccc4c3c2)cc2sc3ccc(N(c4ccccc4)c4ccccc4)cc3c12]

Also, in the present invention, the second host compound represented by Formula (2) comprises the following Compounds 3-1 to 3-124.

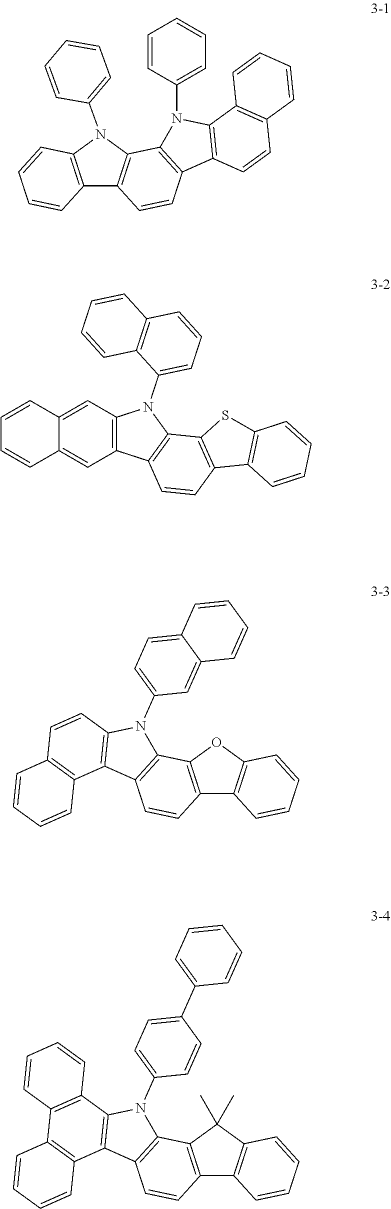 [SMILES: c1ccc(-n2c3ccccc3c3ccc4c5ccc6ccccc6c5n(-c5ccccc5)c4c32)cc1 ; c1ccc2cc3c(cc2c1)c1ccc2c4ccccc4sc2c1n3-c1cccc2ccccc12 ; c1ccc2cc(-n3c4ccc5ccccc5c4c4ccc5c6ccccc6oc5c43)ccc2c1 ; CC1(C)c2ccccc2-c2ccc3c4c5ccccc5c5ccccc5c4n(-c4ccc(-c5ccccc5)cc4)c3c21]

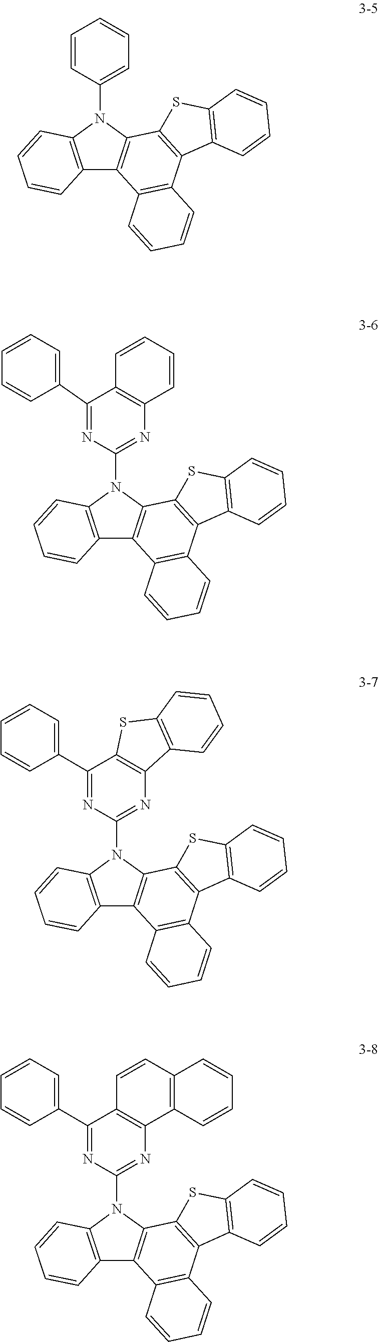 [SMILES: c1ccc(-n2c3ccccc3c3c4ccccc4c4c5ccccc5sc4c32)cc1 ; c1ccc(-c2nc(-n3c4ccccc4c4c5ccccc5c5c6ccccc6sc5c43)nc3ccccc23)cc1 ; c1ccc(-c2nc(-n3c4ccccc4c4c5ccccc5c5c6ccccc6sc5c43)nc3c2sc2ccccc23)cc1 ; c1ccc(-c2nc(-n3c4ccccc4c4c5ccccc5c5c6ccccc6sc5c43)nc3c2ccc2ccccc23)cc1]

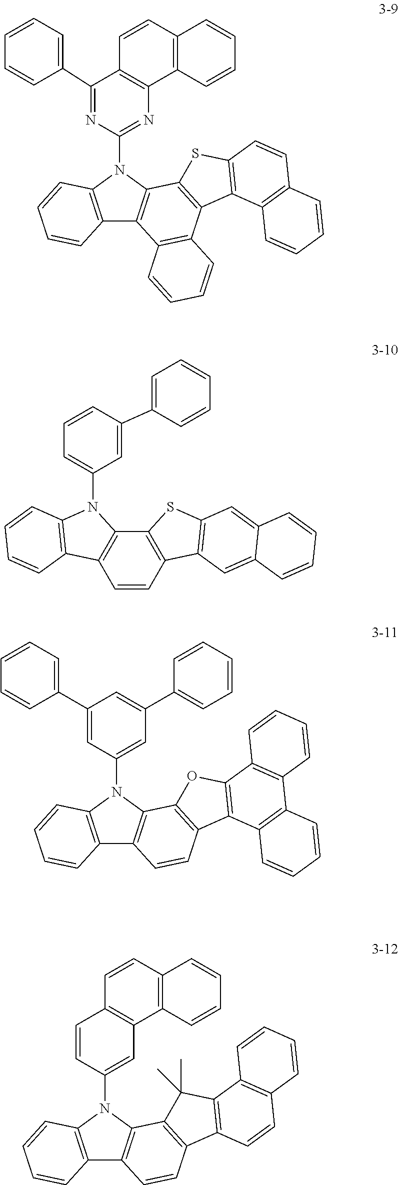 [SMILES: c1ccc(-c2nc(-n3c4ccccc4c4c5ccccc5c5c(sc6ccc7ccccc7c65)c43)nc3c2ccc2ccccc23)cc1 ; c1ccc(-c2cccc(-n3c4ccccc4c4ccc5c6cc7ccccc7cc6sc5c43)c2)cc1 ; c1ccc(-c2cc(-c3ccccc3)cc(-n3c4ccccc4c4ccc5c(oc6c7ccccc7c7ccccc7c56)c43)c2)cc1 ; CC1(C)c2c(ccc3ccccc23)-c2ccc3c4ccccc4n(-c4ccc5ccc6ccccc6c5c4)c3c21]

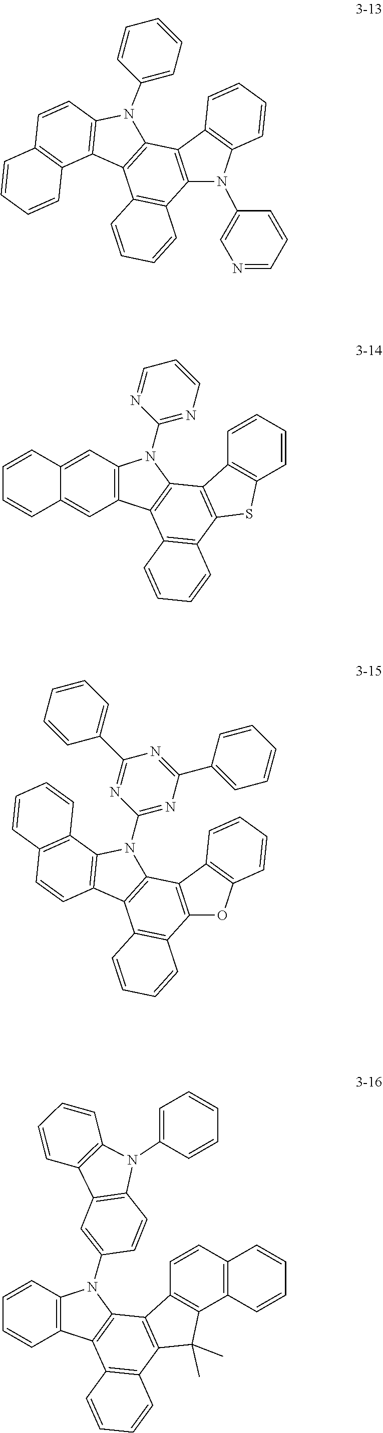 [SMILES: c1ccc(-n2c3ccc4ccccc4c3c3c4ccccc4c4c(c5ccccc5n4-c4cccnc4)c32)cc1 ; c1cnc(-n2c3cc4ccccc4cc3c3c4ccccc4c4sc5ccccc5c4c32)nc1 ; c1ccc(-c2nc(-c3ccccc3)nc(-n3c4c5ccccc5ccc4c4c5ccccc5c5oc6ccccc6c5c43)n2)cc1 ; CC1(C)c2c(ccc3ccccc23)-c2c1c1ccccc1c1c3ccccc3n(-c3ccc4c(c3)c3ccccc3n4-c3ccccc3)c21]

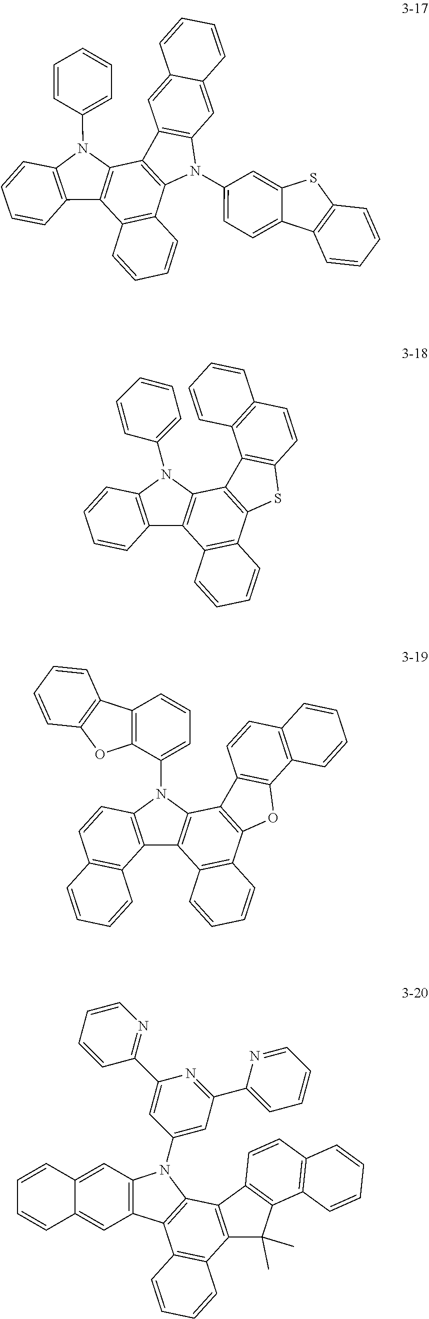 [SMILES: c1ccc(-n2c3ccccc3c3c4ccccc4c4c(c5cc6ccccc6cc5n4-c4ccc5c(c4)sc4ccccc45)c32)cc1 ; c1ccc(-n2c3ccccc3c3c4ccccc4c4sc5ccc6ccccc6c5c4c32)cc1 ; c1ccc2c(c1)ccc1c2oc2c3ccccc3c3c4c5ccccc5ccc4n(-c4cccc5c4oc4ccccc45)c3c12 ; CC1(C)c2c(ccc3ccccc23)-c2c1c1ccccc1c1c3cc4ccccc4cc3n(-c3cc(-c4ccccn4)nc(-c4ccccn4)c3)c21]

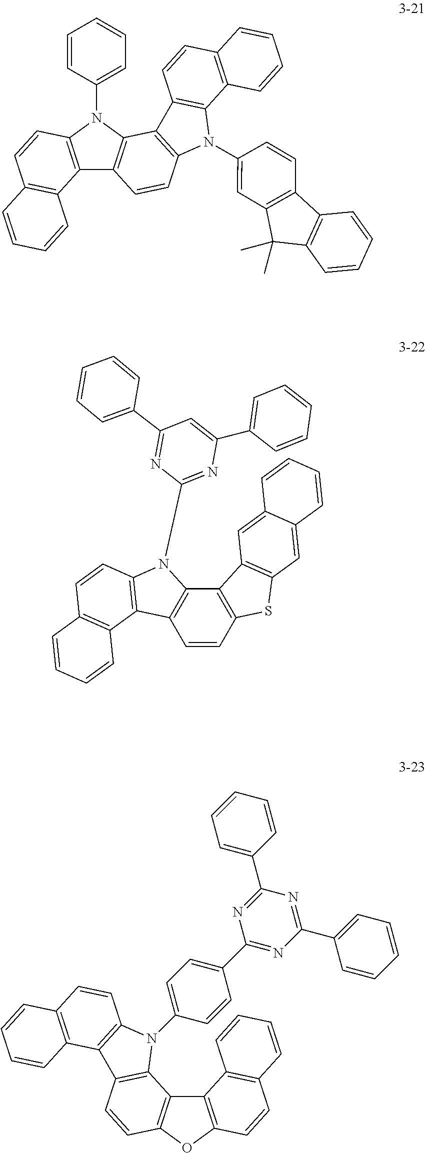 [SMILES: CC1(C)c2ccccc2-c2ccc(-n3c4ccc5c6c7ccccc7ccc6n(-c6ccccc6)c5c4c4ccc5ccccc5c43)cc21 ; c1ccc(-c2cc(-c3ccccc3)nc(-n3c4ccc5ccccc5c4c4ccc5sc6cc7ccccc7cc6c5c43)n2)cc1 ; c1ccc(-c2nc(-c3ccccc3)nc(-c3ccc(-n4c5ccc6ccccc6c5c5ccc6oc7ccc8ccccc8c7c6c54)cc3)n2)cc1]

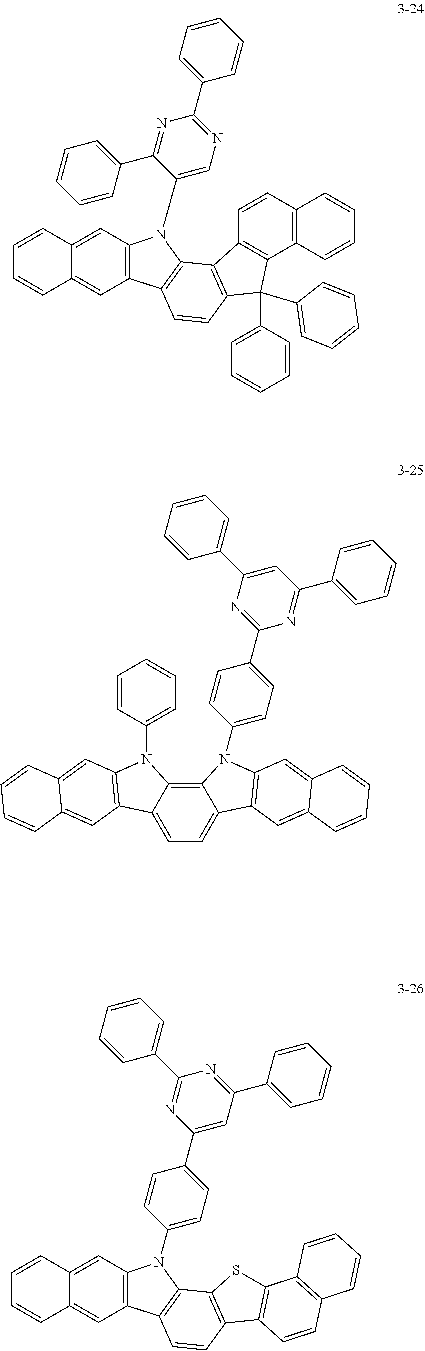 [SMILES: c1ccc(-c2ncc(-n3c4cc5ccccc5cc4c4ccc5c(c43)-c3ccc4ccccc4c3C5(c3ccccc3)c3ccccc3)c(-c3ccccc3)n2)cc1 ; c1ccc(-c2cc(-c3ccccc3)nc(-c3ccc(-n4c5cc6ccccc6cc5c5ccc6c7cc8ccccc8cc7n(-c7ccccc7)c6c54)cc3)n2)cc1 ; c1ccc(-c2cc(-c3ccc(-n4c5cc6ccccc6cc5c5ccc6c7ccc8ccccc8c7sc6c54)cc3)nc(-c3ccccc3)n2)cc1]

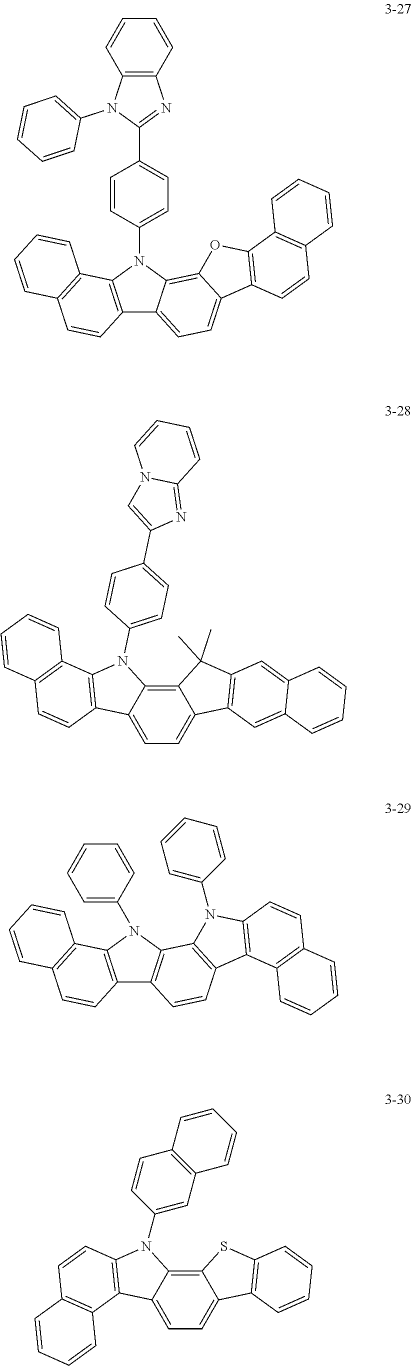 [SMILES: c1ccc(-n2c(-c3ccc(-n4c5c6ccccc6ccc5c5ccc6c7ccc8ccccc8c7oc6c54)cc3)nc3ccccc32)cc1 ; CC1(C)c2cc3ccccc3cc2-c2ccc3c4ccc5ccccc5c4n(-c4ccc(-c5cn6ccccc6n5)cc4)c3c21 ; c1ccc(-n2c3ccc4ccccc4c3c3ccc4c5ccc6ccccc6c5n(-c5ccccc5)c4c32)cc1 ; c1ccc2cc(-n3c4ccc5ccccc5c4c4ccc5c6ccccc6sc5c43)ccc2c1]

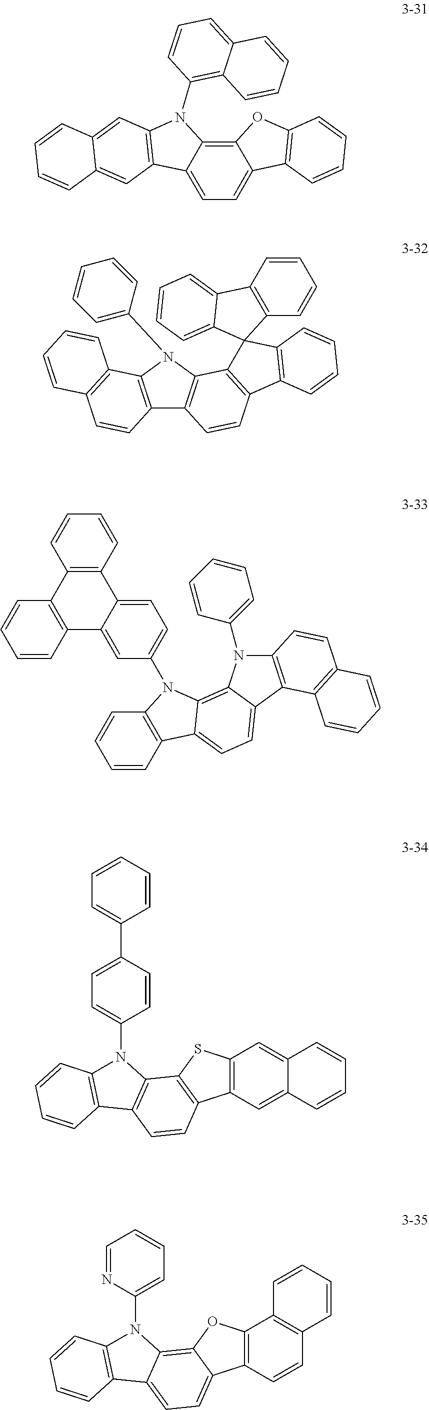 [SMILES: c1ccc2cc3c(cc2c1)c1ccc2c4ccccc4oc2c1n3-c1cccc2ccccc12 ; c1ccc(-n2c3c4c(ccc3c3ccc5ccccc5c32)-c2ccccc2C42c3ccccc3-c3ccccc32)cc1 ; c1ccc(-n2c3ccc4ccccc4c3c3ccc4c5ccccc5n(-c5ccc6c7ccccc7c7ccccc7c6c5)c4c32)cc1 ; c1ccc(-c2ccc(-n3c4ccccc4c4ccc5c6cc7ccccc7cc6sc5c43)cc2)cc1 ; c1ccc(-n2c3ccccc3c3ccc4c5ccc6ccccc6c5oc4c32)nc1]

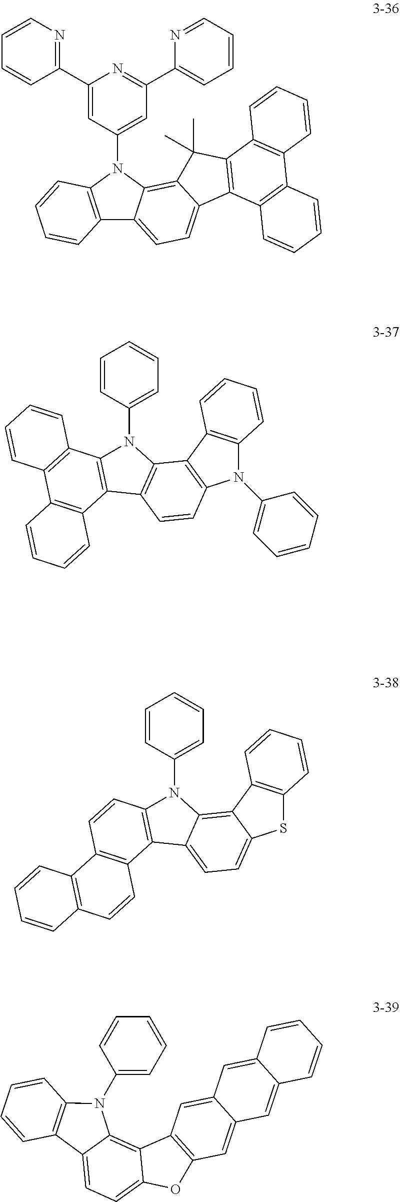 [SMILES: CC1(C)c2c(c3ccccc3c3ccccc23)-c2ccc3c4ccccc4n(-c4cc(-c5ccccn5)nc(-c5ccccn5)c4)c3c21 ; c1ccc(-n2c3ccccc3c3c2ccc2c4c5ccccc5c5ccccc5c4n(-c4ccccc4)c23)cc1 ; c1ccc(-n2c3ccc4c5ccccc5ccc4c3c3ccc4sc5ccccc5c4c32)cc1 ; c1ccc(-n2c3ccccc3c3ccc4oc5cc6cc7ccccc7cc6cc5c4c32)cc1]

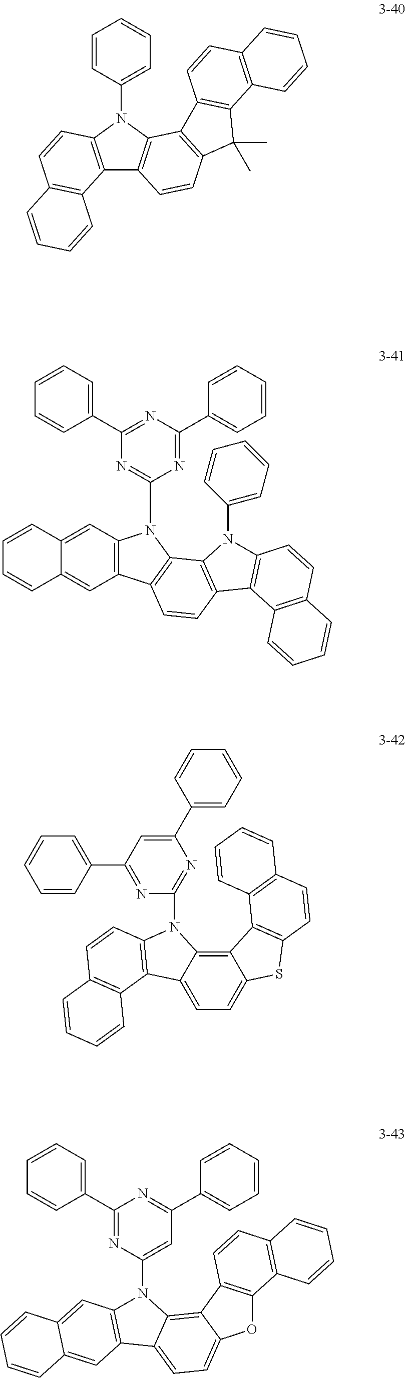 [SMILES: CC1(C)c2ccc3c4c5ccccc5ccc4n(-c4ccccc4)c3c2-c2ccc3ccccc3c21 ; c1ccc(-c2nc(-c3ccccc3)nc(-n3c4cc5ccccc5cc4c4ccc5c6c7ccccc7ccc6n(-c6ccccc6)c5c43)n2)cc1 ; c1ccc(-c2cc(-c3ccccc3)nc(-n3c4ccc5ccccc5c4c4ccc5sc6ccc7ccccc7c6c5c43)n2)cc1 ; c1ccc(-c2cc(-n3c4cc5ccccc5cc4c4ccc5oc6c7ccccc7ccc6c5c43)nc(-c3ccccc3)n2)cc1]

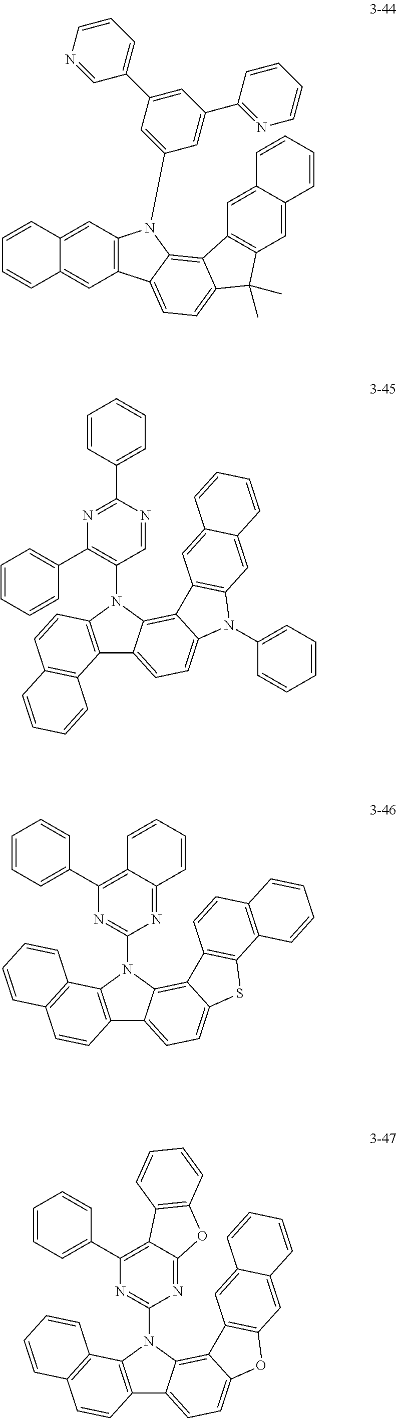 [SMILES: CC1(C)c2cc3ccccc3cc2-c2c1ccc1c3cc4ccccc4cc3n(-c3cc(-c4cccnc4)cc(-c4ccccn4)c3)c21 ; c1ccc(-c2ncc(-n3c4ccc5ccccc5c4c4ccc5c(c6cc7ccccc7cc6n5-c5ccccc5)c43)c(-c3ccccc3)n2)cc1 ; c1ccc(-c2nc(-n3c4c5ccccc5ccc4c4ccc5sc6c7ccccc7ccc6c5c43)nc3ccccc23)cc1 ; c1ccc(-c2nc(-n3c4c5ccccc5ccc4c4ccc5oc6cc7ccccc7cc6c5c43)nc3oc4ccccc4c23)cc1]

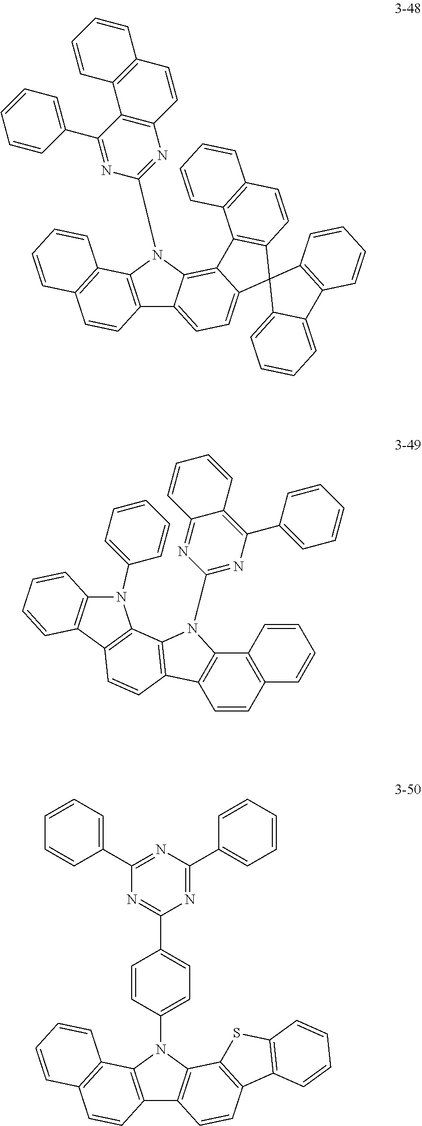 [SMILES: c1ccc(-c2nc(-n3c4c5c(ccc4c4ccc6ccccc6c43)C3(c4ccccc4-c4ccccc43)c3ccc4ccccc4c3-5)nc3ccc4ccccc4c23)cc1 ; c1ccc(-c2nc(-n3c4c5ccccc5ccc4c4ccc5c6ccccc6n(-c6ccccc6)c5c43)nc3ccccc23)cc1 ; c1ccc(-c2nc(-c3ccccc3)nc(-c3ccc(-n4c5c6ccccc6ccc5c5ccc6c7ccccc7sc6c54)cc3)n2)cc1]

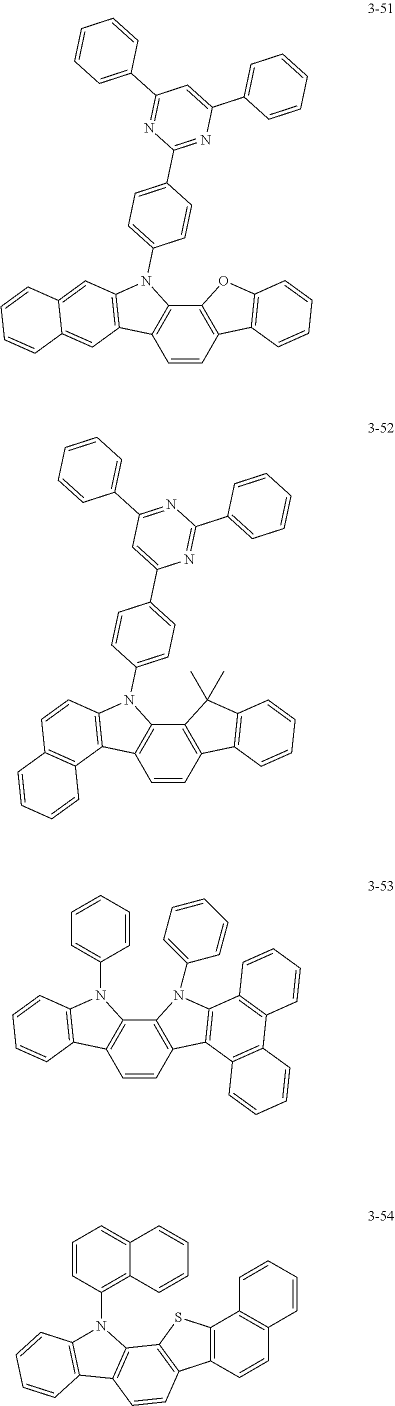 [SMILES: c1ccc(-c2cc(-c3ccccc3)nc(-c3ccc(-n4c5cc6ccccc6cc5c5ccc6c7ccccc7oc6c54)cc3)n2)cc1 ; CC1(C)c2ccccc2-c2ccc3c4c5ccccc5ccc4n(-c4ccc(-c5cc(-c6ccccc6)nc(-c6ccccc6)n5)cc4)c3c21 ; c1ccc(-n2c3ccccc3c3ccc4c5c6ccccc6c6ccccc6c5n(-c5ccccc5)c4c32)cc1 ; c1ccc2c(-n3c4ccccc4c4ccc5c6ccc7ccccc7c6sc5c43)cccc2c1]

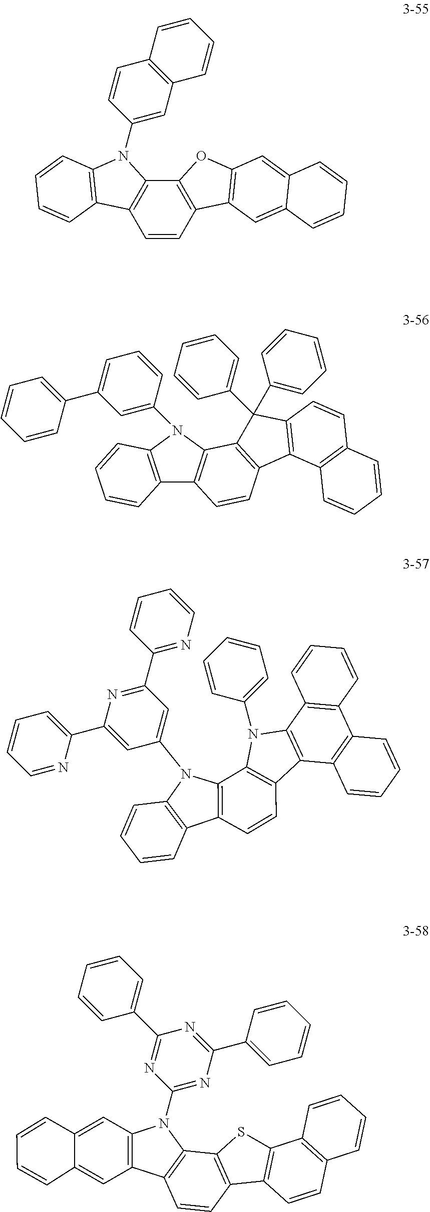 [SMILES: c1ccc2cc(-n3c4ccccc4c4ccc5c6cc7ccccc7cc6oc5c43)ccc2c1 ; c1ccc(-c2cccc(-n3c4ccccc4c4ccc5c(c43)C(c3ccccc3)(c3ccccc3)c3ccc4ccccc4c3-5)c2)cc1 ; c1ccc(-n2c3c4ccccc4c4ccccc4c3c3ccc4c5ccccc5n(-c5cc(-c6ccccn6)nc(-c6ccccn6)c5)c4c32)cc1 ; c1ccc(-c2nc(-c3ccccc3)nc(-n3c4cc5ccccc5cc4c4ccc5c6ccc7ccccc7c6sc5c43)n2)cc1]

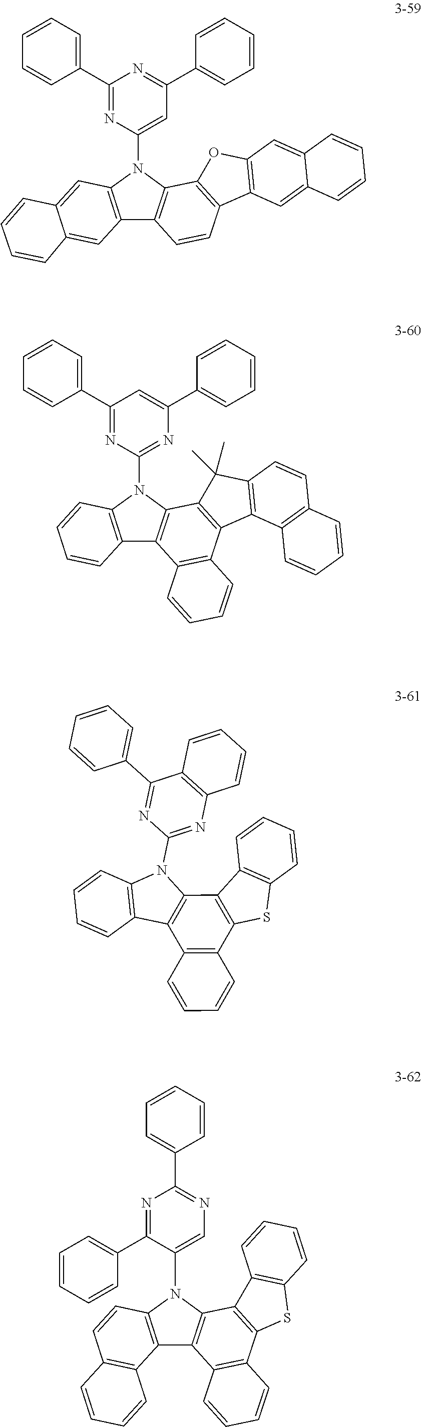 [SMILES: c1ccc(-c2cc(-n3c4cc5ccccc5cc4c4ccc5c6cc7ccccc7cc6oc5c43)nc(-c3ccccc3)n2)cc1 ; CC1(C)c2ccc3ccccc3c2-c2c1c1c(c3ccccc23)c2ccccc2n1-c1nc(-c2ccccc2)cc(-c2ccccc2)n1 ; c1ccc(-c2nc(-n3c4ccccc4c4c5ccccc5c5sc6ccccc6c5c43)nc3ccccc23)cc1 ; c1ccc(-c2ncc(-n3c4ccc5ccccc5c4c4c5ccccc5c5sc6ccccc6c5c43)c(-c3ccccc3)n2)cc1]

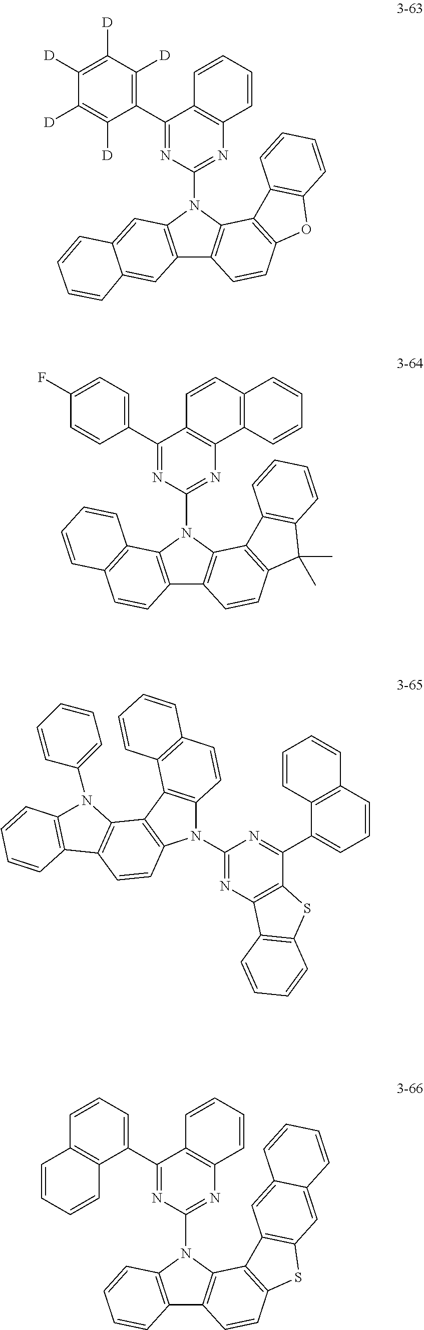 [SMILES: [2H]c1c([2H])c([2H])c(-c2nc(-n3c4cc5ccccc5cc4c4ccc5oc6ccccc6c5c43)nc3ccccc23)c([2H])c1[2H] ; CC1(C)c2ccccc2-c2c1ccc1c3ccc4ccccc4c3n(-c3nc(-c4ccc(F)cc4)c4ccc5ccccc5c4n3)c21 ; c1ccc(-n2c3ccccc3c3ccc4c(c5c6ccccc6ccc5n4-c4nc(-c5cccc6ccccc56)c5sc6ccccc6c5n4)c32)cc1 ; c1ccc2cc3c(cc2c1)sc1ccc2c4ccccc4n(-c4nc(-c5cccc6ccccc56)c5ccccc5n4)c2c13]

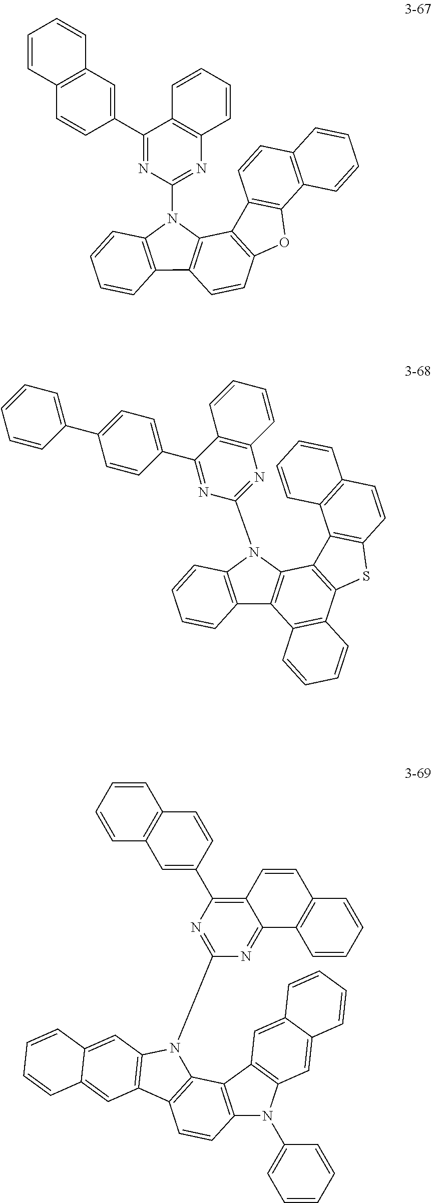 [SMILES: c1ccc2cc(-c3nc(-n4c5ccccc5c5ccc6oc7c8ccccc8ccc7c6c54)nc4ccccc34)ccc2c1 ; c1ccc(-c2ccc(-c3nc(-n4c5ccccc5c5c6ccccc6c6sc7ccc8ccccc8c7c6c54)nc4ccccc34)cc2)cc1 ; c1ccc(-n2c3cc4ccccc4cc3c3c2ccc2c4cc5ccccc5cc4n(-c4nc(-c5ccc6ccccc6c5)c5ccc6ccccc6c5n4)c23)cc1]

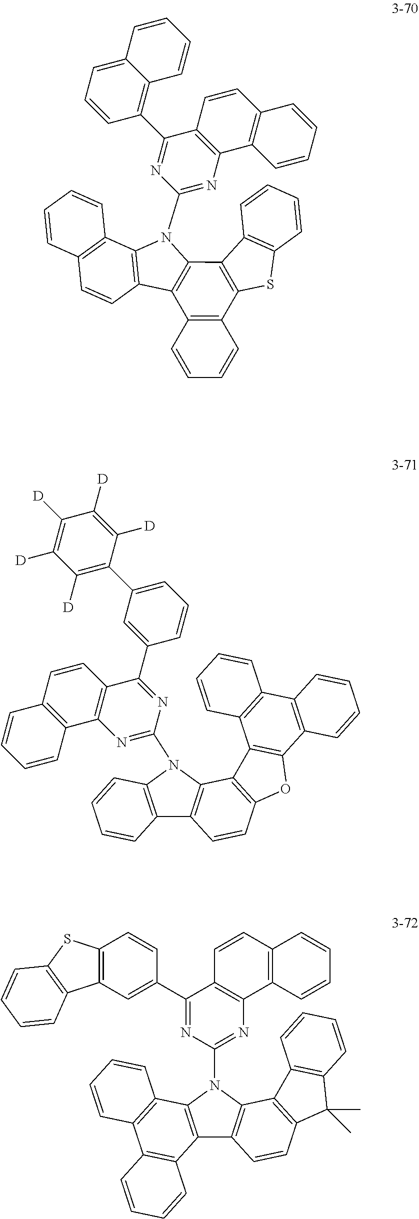 [SMILES: c1ccc2c(-c3nc(-n4c5c6ccccc6ccc5c5c6ccccc6c6sc7ccccc7c6c54)nc4c3ccc3ccccc34)cccc2c1 ; [2H]c1c([2H])c([2H])c(-c2cccc(-c3nc(-n4c5ccccc5c5ccc6oc7c8ccccc8c8ccccc8c7c6c54)nc4c3ccc3ccccc34)c2)c([2H])c1[2H] ; CC1(C)c2ccccc2-c2c1ccc1c3c4ccccc4c4ccccc4c3n(-c3nc(-c4ccc5sc6ccccc6c5c4)c4ccc5ccccc5c4n3)c21]

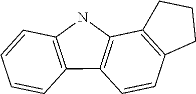 [SMILES: c1ccc2c(c1)[nH]c1c3c(ccc12)CCC3]

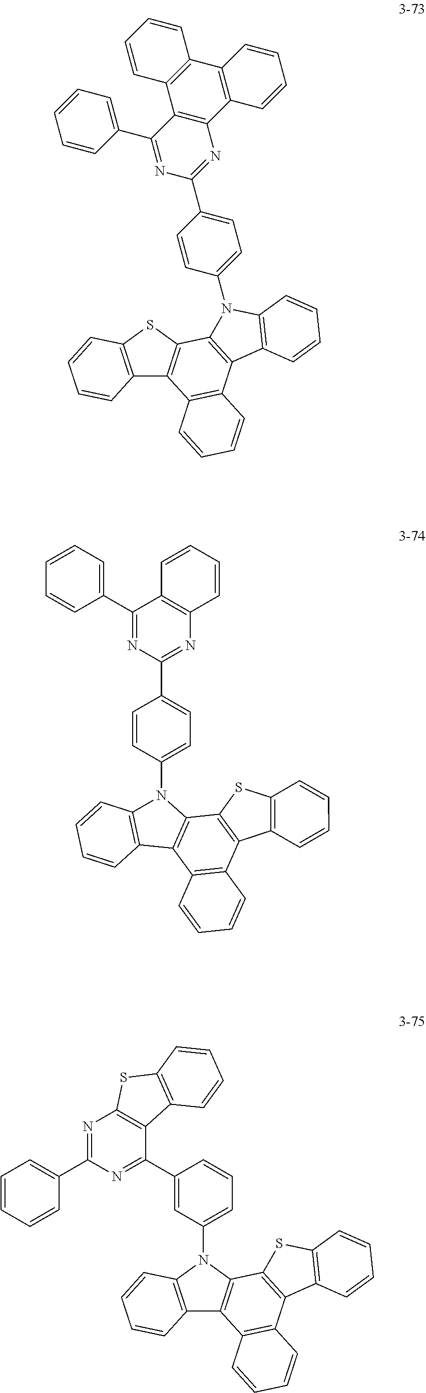 [SMILES: c1ccc(-c2nc(-c3ccc(-n4c5ccccc5c5c6ccccc6c6c7ccccc7sc6c54)cc3)nc3c4ccccc4c4ccccc4c23)cc1 ; c1ccc(-c2nc(-c3ccc(-n4c5ccccc5c5c6ccccc6c6c7ccccc7sc6c54)cc3)nc3ccccc23)cc1 ; c1ccc(-c2nc(-c3cccc(-n4c5ccccc5c5c6ccccc6c6c7ccccc7sc6c54)c3)c3c(n2)sc2ccccc23)cc1]

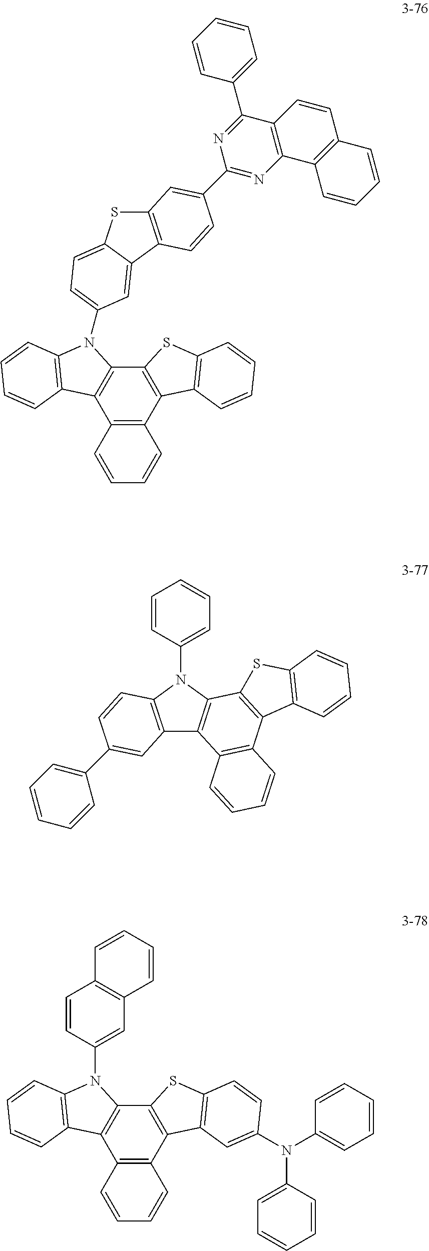 [SMILES: c1ccc(-c2nc(-c3ccc4c(c3)sc3ccc(-n5c6ccccc6c6c7ccccc7c7c8ccccc8sc7c65)cc34)nc3c2ccc2ccccc23)cc1 ; c1ccc(-c2ccc3c(c2)c2c4ccccc4c4c5ccccc5sc4c2n3-c2ccccc2)cc1 ; c1ccc(N(c2ccccc2)c2ccc3sc4c(c3c2)c2ccccc2c2c3ccccc3n(-c3ccc5ccccc5c3)c42)cc1]

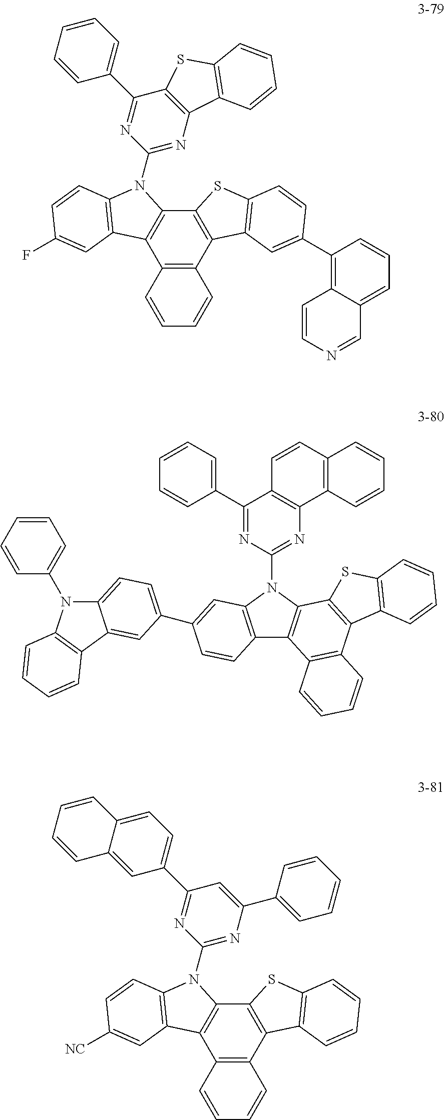 [SMILES: Fc1ccc2c(c1)c1c3ccccc3c3c4cc(-c5cccc6cnccc56)ccc4sc3c1n2-c1nc(-c2ccccc2)c2sc3ccccc3c2n1 ; c1ccc(-c2nc(-n3c4cc(-c5ccc6c(c5)c5ccccc5n6-c5ccccc5)ccc4c4c5ccccc5c5c6ccccc6sc5c43)nc3c2ccc2ccccc23)cc1 ; N#Cc1ccc2c(c1)c1c3ccccc3c3c4ccccc4sc3c1n2-c1nc(-c2ccccc2)cc(-c2ccc3ccccc3c2)n1]

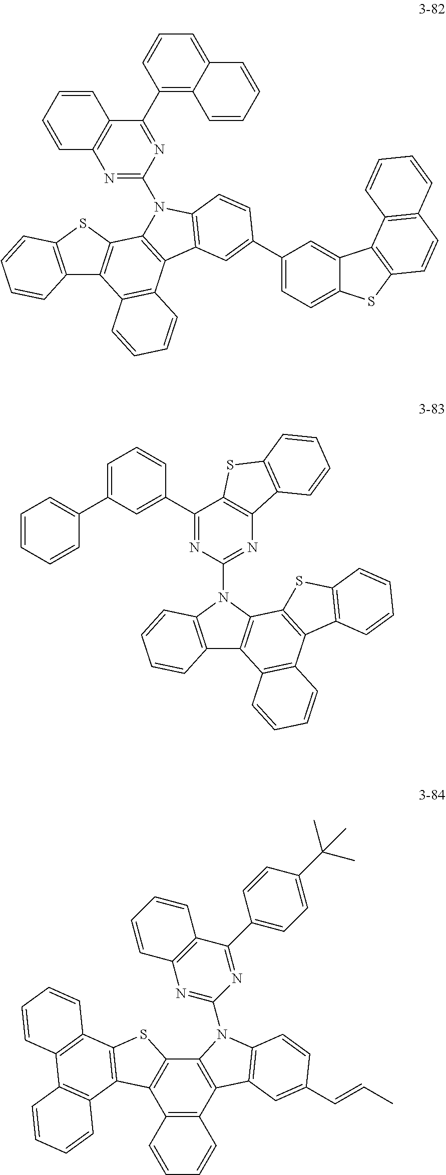 [SMILES: c1ccc2c(-c3nc(-n4c5ccc(-c6ccc7sc8ccc9ccccc9c8c7c6)cc5c5c6ccccc6c6c7ccccc7sc6c54)nc4ccccc34)cccc2c1 ; c1ccc(-c2cccc(-c3nc(-n4c5ccccc5c5c6ccccc6c6c7ccccc7sc6c54)nc4c3sc3ccccc34)c2)cc1 ; C/C=C/c1ccc2c(c1)c1c3ccccc3c3c(sc4c5ccccc5c5ccccc5c43)c1n2-c1nc(-c2ccc(C(C)(C)C)cc2)c2ccccc2n1]

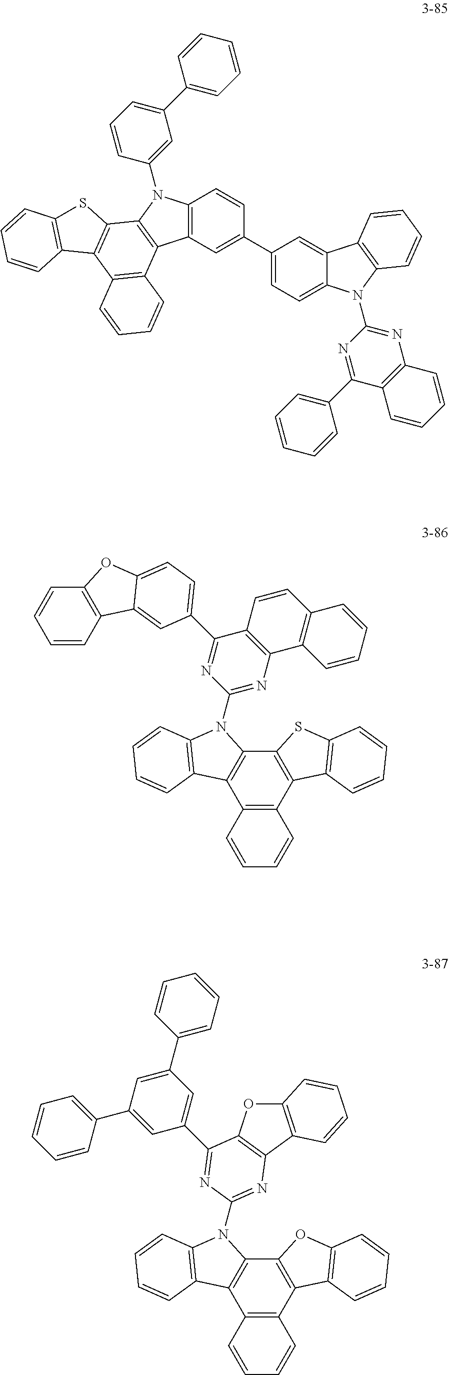 [SMILES: c1ccc(-c2cccc(-n3c4ccc(-c5ccc6c(c5)c5ccccc5n6-c5nc(-c6ccccc6)c6ccccc6n5)cc4c4c5ccccc5c5c6ccccc6sc5c43)c2)cc1 ; c1ccc2c(c1)ccc1c(-c3ccc4oc5ccccc5c4c3)nc(-n3c4ccccc4c4c5ccccc5c5c6ccccc6sc5c43)nc12 ; c1ccc(-c2cc(-c3ccccc3)cc(-c3nc(-n4c5ccccc5c5c6ccccc6c6c7ccccc7oc6c54)nc4c3oc3ccccc34)c2)cc1]

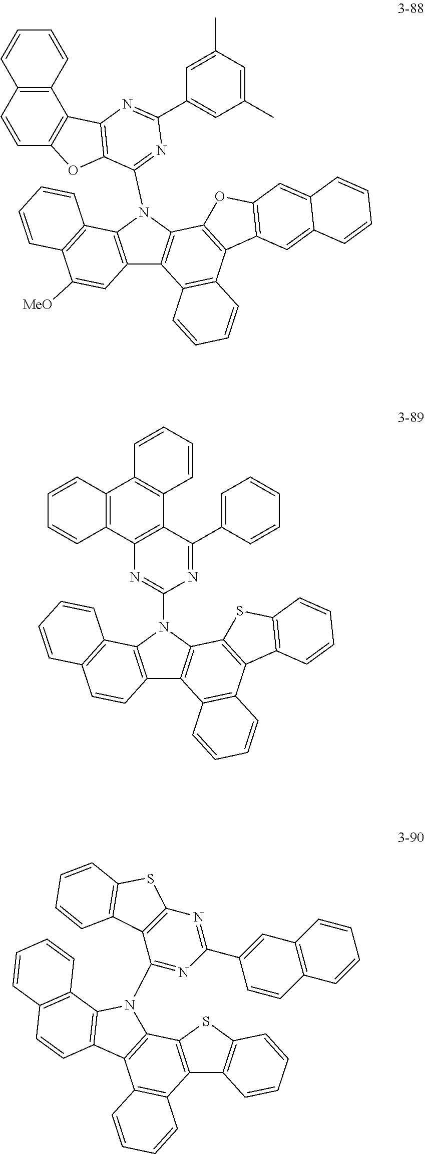 [SMILES: COc1cc2c3c4ccccc4c4c5cc6ccccc6cc5oc4c3n(-c3nc(-c4cc(C)cc(C)c4)nc4c3oc3ccc5ccccc5c34)c2c2ccccc12 ; c1ccc(-c2nc(-n3c4c5ccccc5ccc4c4c5ccccc5c5c6ccccc6sc5c43)nc3c4ccccc4c4ccccc4c23)cc1 ; c1ccc2cc(-c3nc(-n4c5c6ccccc6ccc5c5c6ccccc6c6c7ccccc7sc6c54)c4c(n3)sc3ccccc34)ccc2c1]

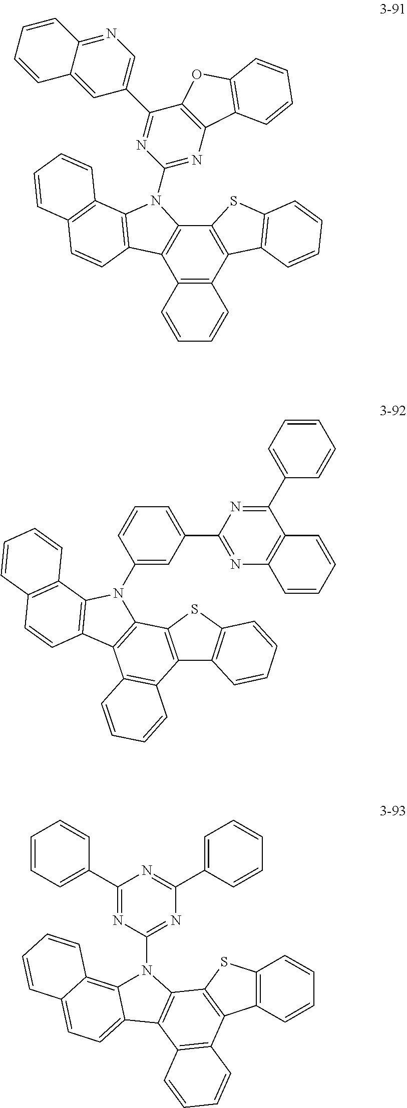 [SMILES: c1ccc2ncc(-c3nc(-n4c5c6ccccc6ccc5c5c6ccccc6c6c7ccccc7sc6c54)nc4c3oc3ccccc34)cc2c1 ; c1ccc(-c2nc(-c3cccc(-n4c5c6ccccc6ccc5c5c6ccccc6c6c7ccccc7sc6c54)c3)nc3ccccc23)cc1 ; c1ccc(-c2nc(-c3ccccc3)nc(-n3c4c5ccccc5ccc4c4c5ccccc5c5c6ccccc6sc5c43)n2)cc1]

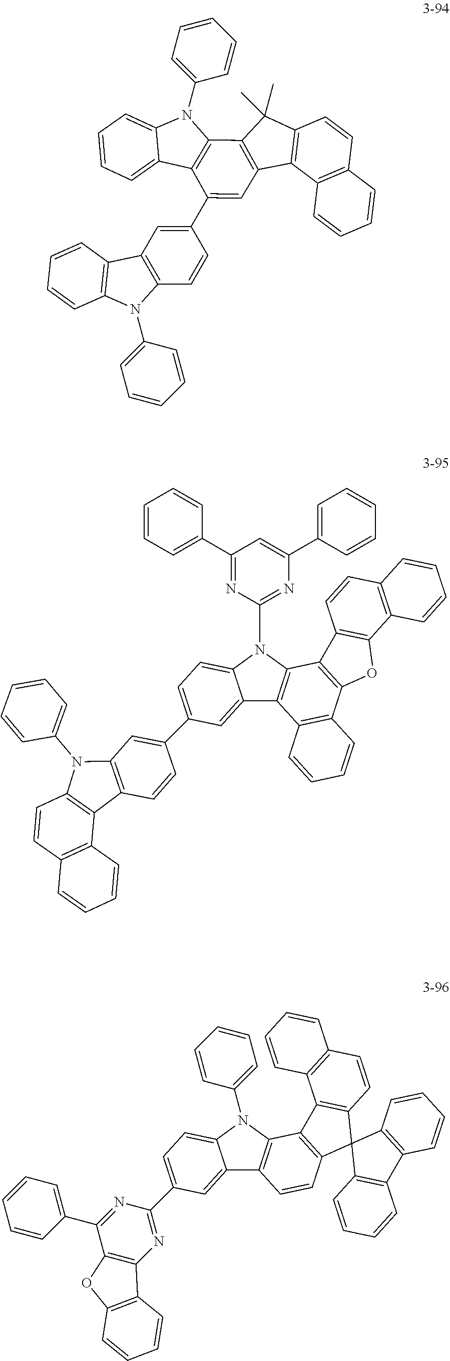 [SMILES: CC1(C)c2ccc3ccccc3c2-c2cc(-c3ccc4c(c3)c3ccccc3n4-c3ccccc3)c3c4ccccc4n(-c4ccccc4)c3c21 ; c1ccc(-c2cc(-c3ccccc3)nc(-n3c4ccc(-c5ccc6c7c8ccccc8ccc7n(-c7ccccc7)c6c5)cc4c4c5ccccc5c5oc6c7ccccc7ccc6c5c43)n2)cc1 ; c1ccc(-c2nc(-c3ccc4c(c3)c3ccc5c(c3n4-c3ccccc3)-c3c(ccc4ccccc34)C53c4ccccc4-c4ccccc43)nc3c2oc2ccccc23)cc1]

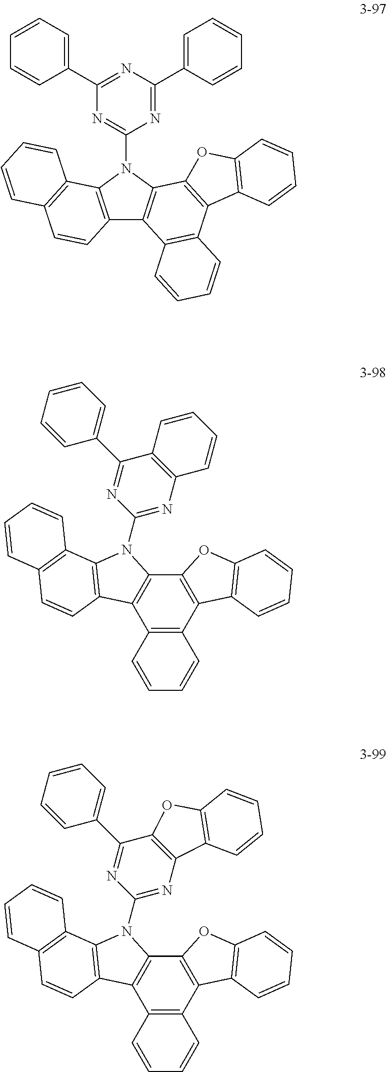 [SMILES: c1ccc(-c2nc(-c3ccccc3)nc(-n3c4c5ccccc5ccc4c4c5ccccc5c5c6ccccc6oc5c43)n2)cc1 ; c1ccc(-c2nc(-n3c4c5ccccc5ccc4c4c5ccccc5c5c6ccccc6oc5c43)nc3ccccc23)cc1 ; c1ccc(-c2nc(-n3c4c5ccccc5ccc4c4c5ccccc5c5c6ccccc6oc5c43)nc3c2oc2ccccc23)cc1]

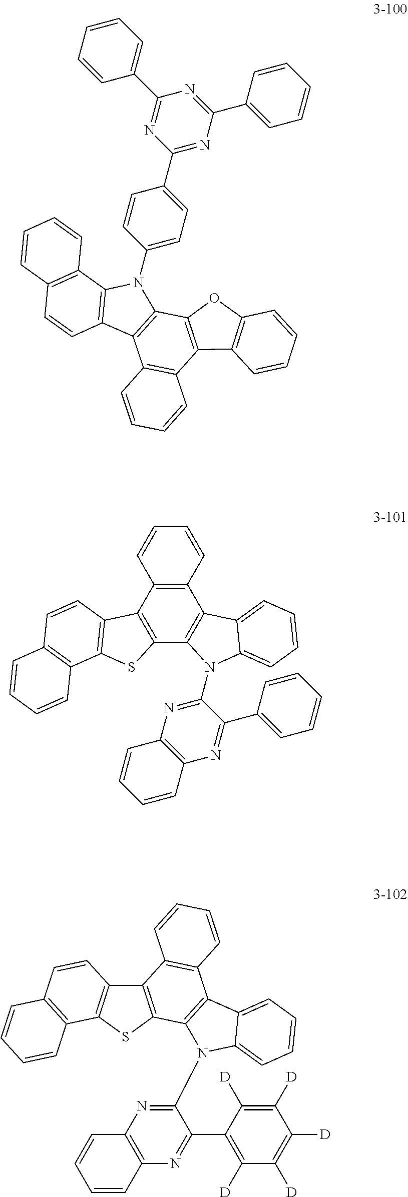 [SMILES: c1ccc(-c2nc(-c3ccccc3)nc(-c3ccc(-n4c5c6ccccc6ccc5c5c6ccccc6c6c7ccccc7oc6c54)cc3)n2)cc1 ; c1ccc(-c2nc3ccccc3nc2-n2c3ccccc3c3c4ccccc4c4c5ccc6ccccc6c5sc4c32)cc1 ; [2H]c1c([2H])c([2H])c(-c2nc3ccccc3nc2-n2c3ccccc3c3c4ccccc4c4c5ccc6ccccc6c5sc4c32)c([2H])c1[2H]]

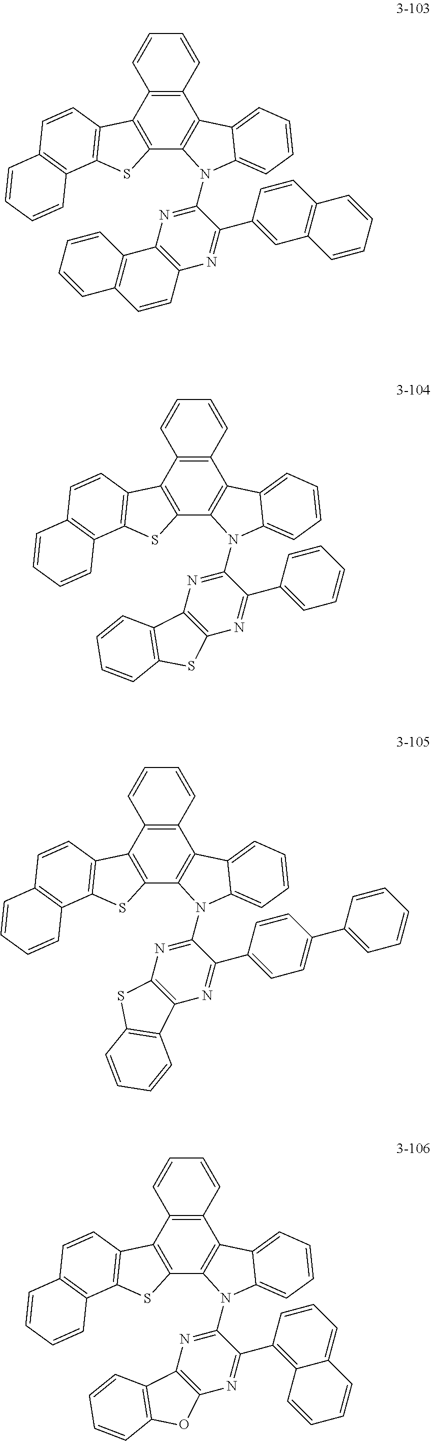 [SMILES: c1ccc2cc(-c3nc4ccc5ccccc5c4nc3-n3c4ccccc4c4c5ccccc5c5c6ccc7ccccc7c6sc5c43)ccc2c1 ; c1ccc(-c2nc3sc4ccccc4c3nc2-n2c3ccccc3c3c4ccccc4c4c5ccc6ccccc6c5sc4c32)cc1 ; c1ccc(-c2ccc(-c3nc4c(nc3-n3c5ccccc5c5c6ccccc6c6c7ccc8ccccc8c7sc6c53)sc3ccccc34)cc2)cc1 ; c1ccc2c(-c3nc4oc5ccccc5c4nc3-n3c4ccccc4c4c5ccccc5c5c6ccc7ccccc7c6sc5c43)cccc2c1]

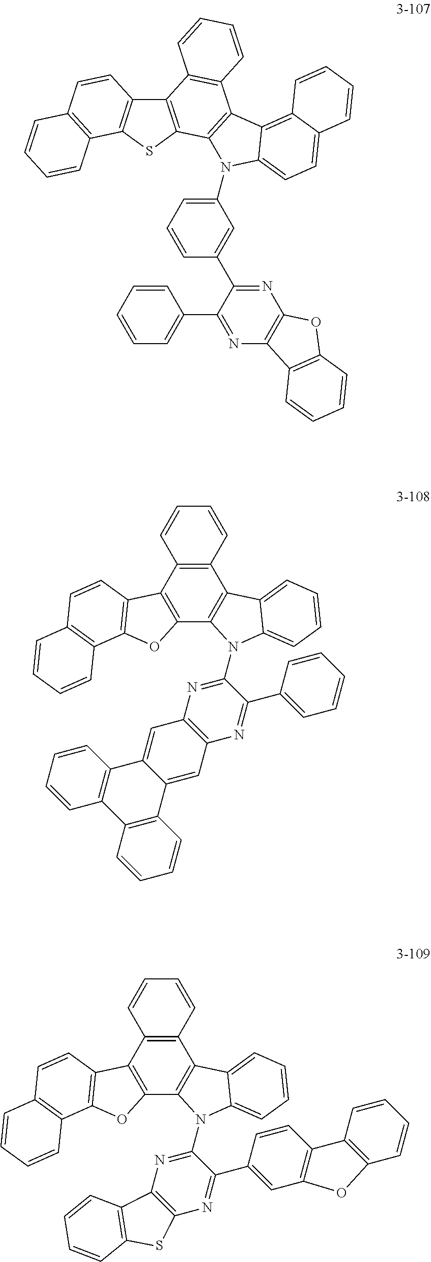 [SMILES: c1ccc(-c2nc3c(nc2-c2cccc(-n4c5ccc6ccccc6c5c5c6ccccc6c6c7ccc8ccccc8c7sc6c54)c2)oc2ccccc23)cc1 ; c1ccc(-c2nc3cc4c5ccccc5c5ccccc5c4cc3nc2-n2c3ccccc3c3c4ccccc4c4c5ccc6ccccc6c5oc4c32)cc1 ; c1ccc2c(c1)ccc1c2oc2c1c1ccccc1c1c3ccccc3n(-c3nc4c(nc3-c3ccc5c(c3)oc3ccccc35)sc3ccccc34)c21]

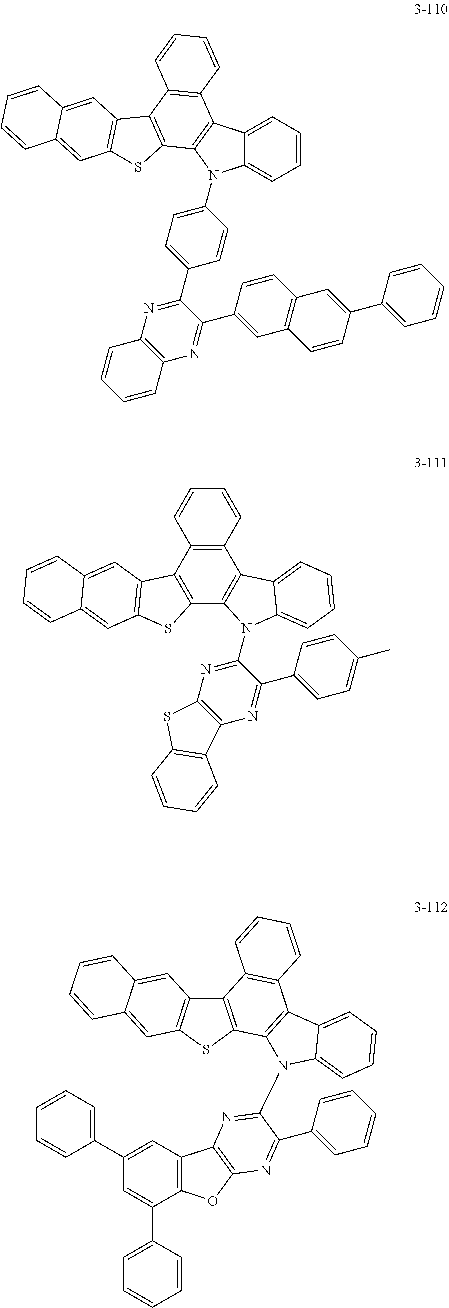 [SMILES: c1ccc(-c2ccc3cc(-c4nc5ccccc5nc4-c4ccc(-n5c6ccccc6c6c7ccccc7c7c8cc9ccccc9cc8sc7c65)cc4)ccc3c2)cc1 ; Cc1ccc(-c2nc3c(nc2-n2c4ccccc4c4c5ccccc5c5c6cc7ccccc7cc6sc5c42)sc2ccccc23)cc1 ; c1ccc(-c2cc(-c3ccccc3)c3oc4nc(-c5ccccc5)c(-n5c6ccccc6c6c7ccccc7c7c8cc9ccccc9cc8sc7c65)nc4c3c2)cc1]

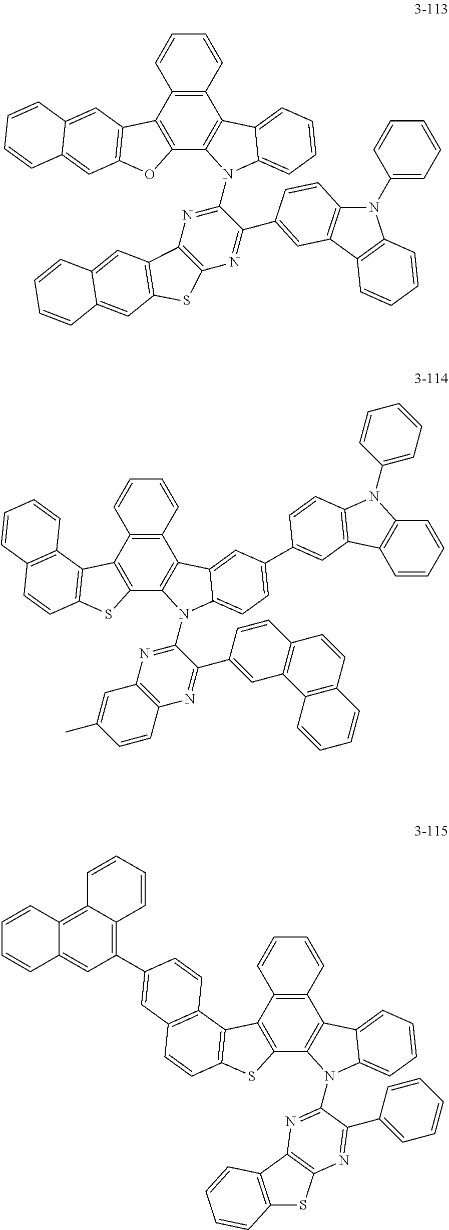 [SMILES: c1ccc(-n2c3ccccc3c3cc(-c4nc5sc6cc7ccccc7cc6c5nc4-n4c5ccccc5c5c6ccccc6c6c7cc8ccccc8cc7oc6c54)ccc32)cc1 ; Cc1ccc2nc(-c3ccc4ccc5ccccc5c4c3)c(-n3c4ccc(-c5ccc6c(c5)c5ccccc5n6-c5ccccc5)cc4c4c5ccccc5c5c(sc6ccc7ccccc7c65)c43)nc2c1 ; c1ccc(-c2nc3sc4ccccc4c3nc2-n2c3ccccc3c3c4ccccc4c4c(sc5ccc6cc(-c7cc8ccccc8c8ccccc78)ccc6c54)c32)cc1]

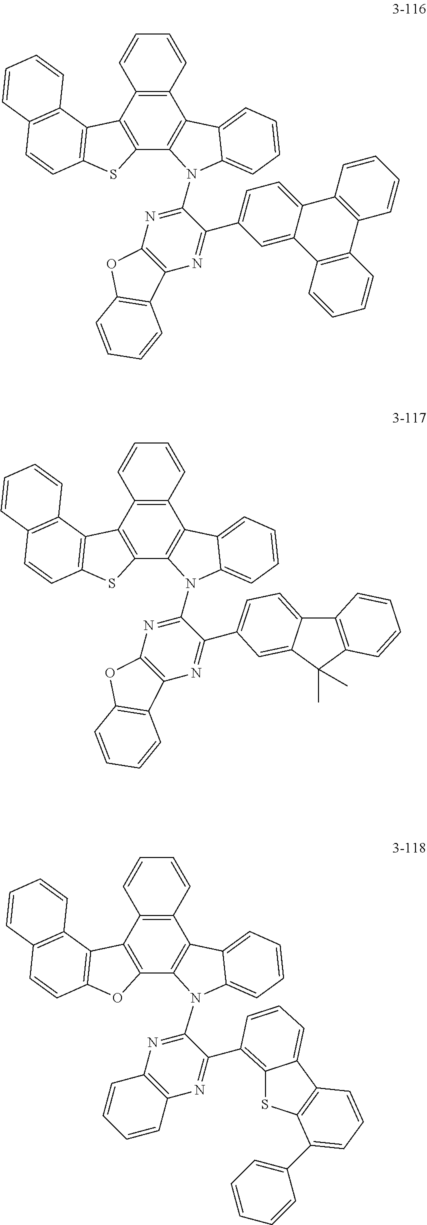 [SMILES: c1ccc2c(c1)ccc1sc3c(c4ccccc4c4c5ccccc5n(-c5nc6oc7ccccc7c6nc5-c5ccc6c7ccccc7c7ccccc7c6c5)c34)c12 ; CC1(C)c2ccccc2-c2ccc(-c3nc4c(nc3-n3c5ccccc5c5c6ccccc6c6c(sc7ccc8ccccc8c76)c53)oc3ccccc34)cc21 ; c1ccc(-c2cccc3c2sc2c(-c4nc5ccccc5nc4-n4c5ccccc5c5c6ccccc6c6c(oc7ccc8ccccc8c76)c54)cccc23)cc1]

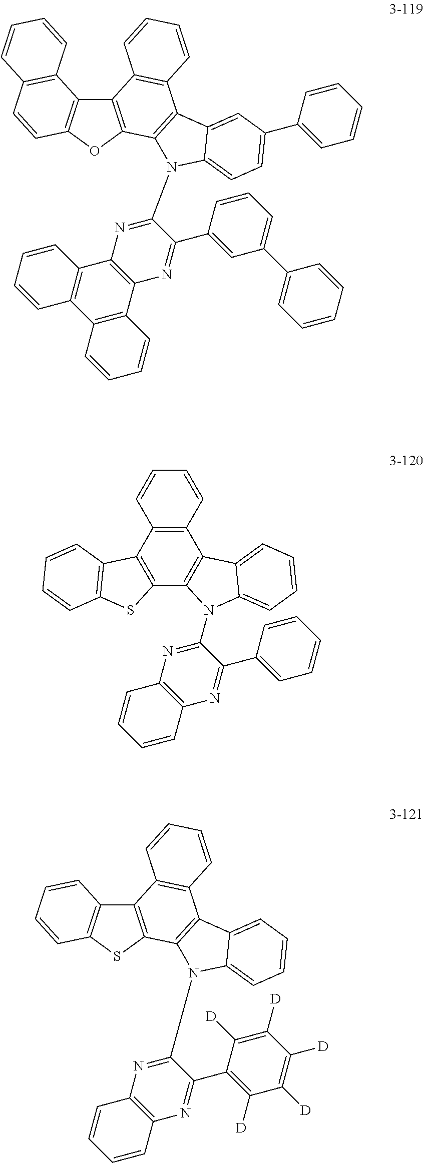 [SMILES: c1ccc(-c2cccc(-c3nc4c5ccccc5c5ccccc5c4nc3-n3c4ccc(-c5ccccc5)cc4c4c5ccccc5c5c(oc6ccc7ccccc7c65)c43)c2)cc1 ; c1ccc(-c2nc3ccccc3nc2-n2c3ccccc3c3c4ccccc4c4c5ccccc5sc4c32)cc1 ; [2H]c1c([2H])c([2H])c(-c2nc3ccccc3nc2-n2c3ccccc3c3c4ccccc4c4c5ccccc5sc4c32)c([2H])c1[2H]]

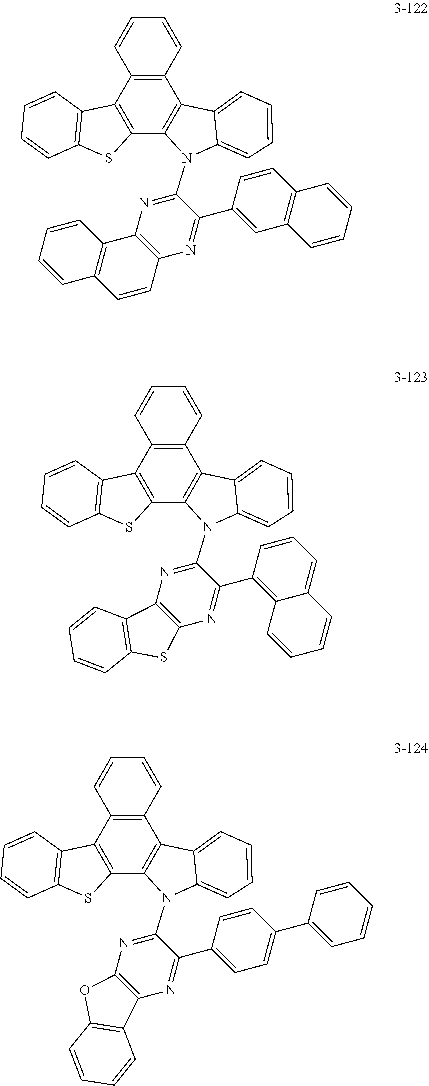 [SMILES: c1ccc2cc(-c3nc4ccc5ccccc5c4nc3-n3c4ccccc4c4c5ccccc5c5c6ccccc6sc5c43)ccc2c1 ; c1ccc2c(-c3nc4sc5ccccc5c4nc3-n3c4ccccc4c4c5ccccc5c5c6ccccc6sc5c43)cccc2c1 ; c1ccc(-c2ccc(-c3nc4c(nc3-n3c5ccccc5c5c6ccccc6c6c7ccccc7sc6c53)oc3ccccc34)cc2)cc1]

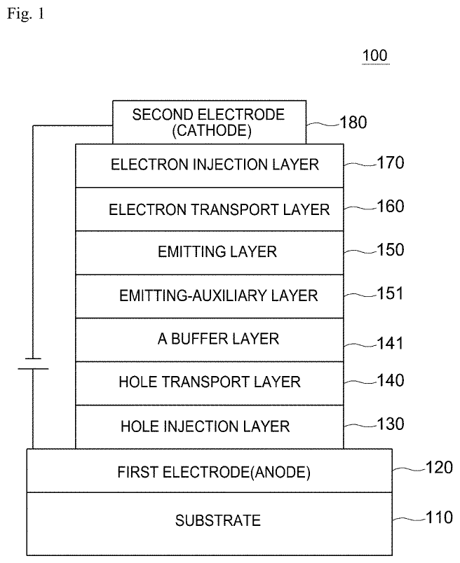

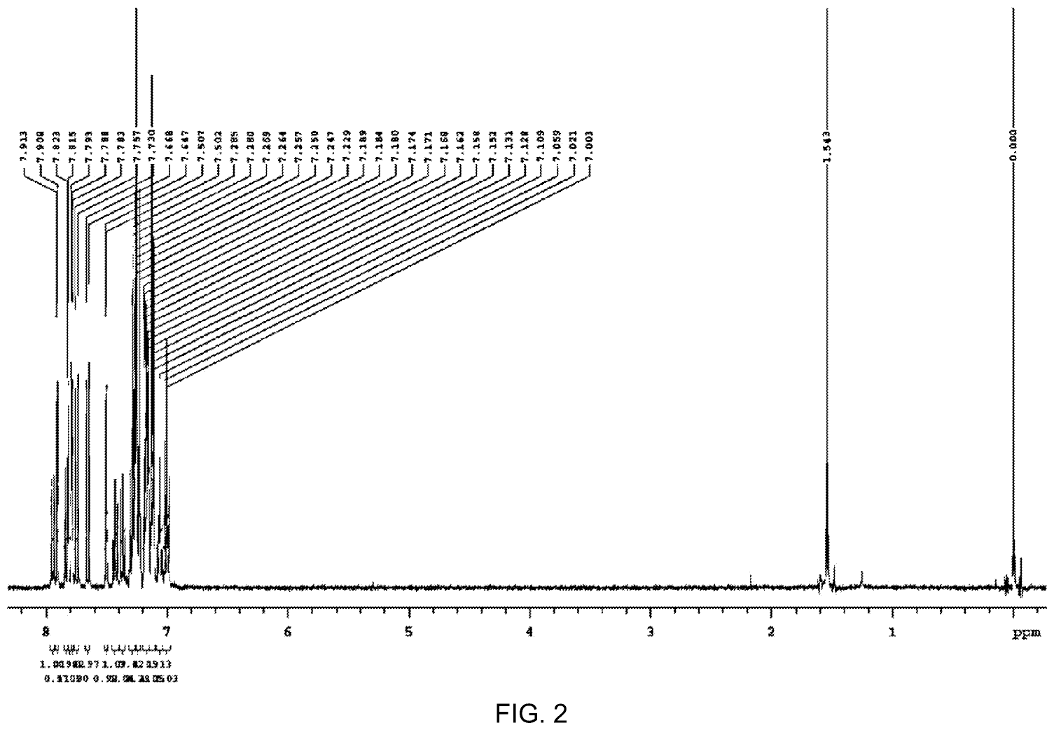

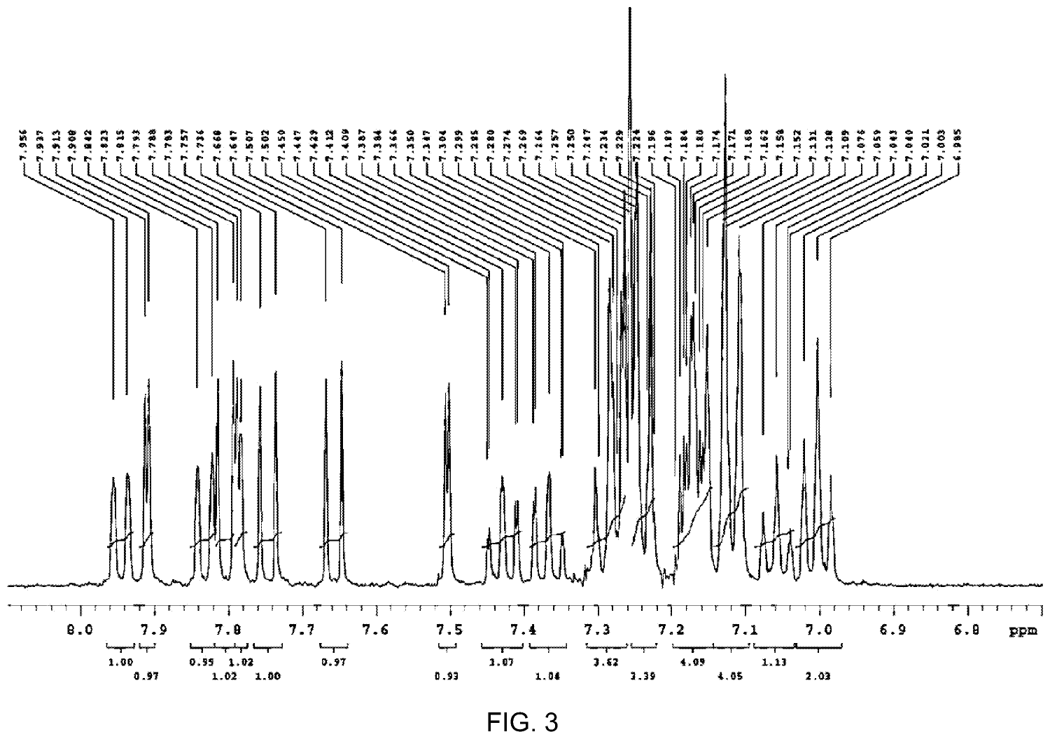

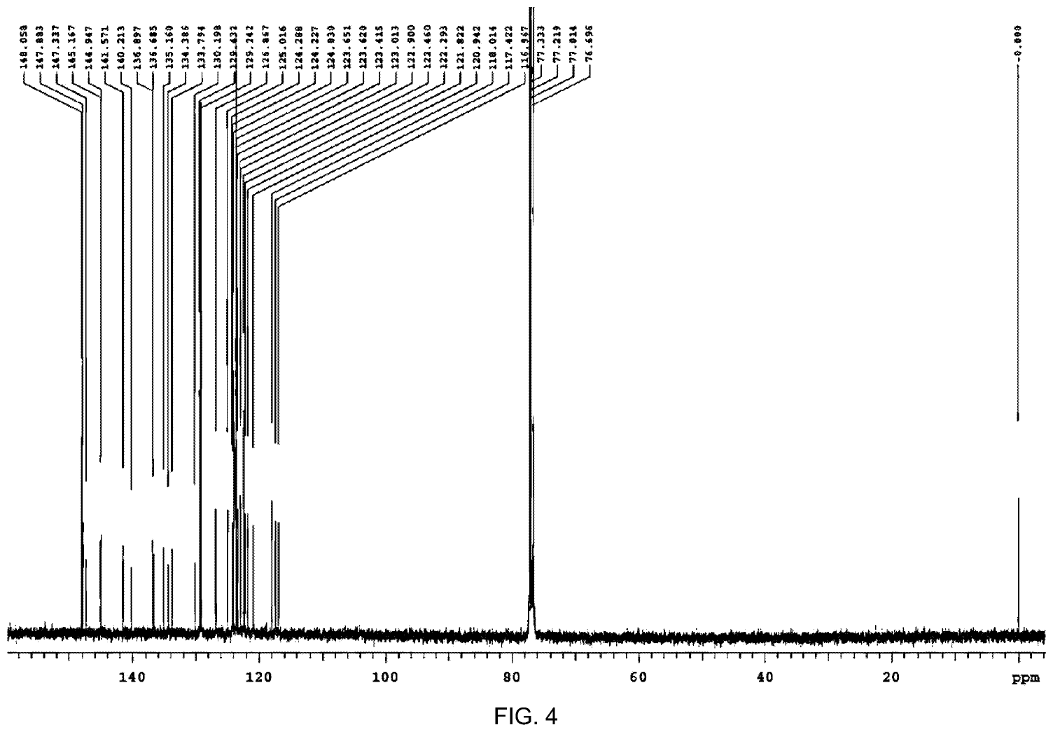

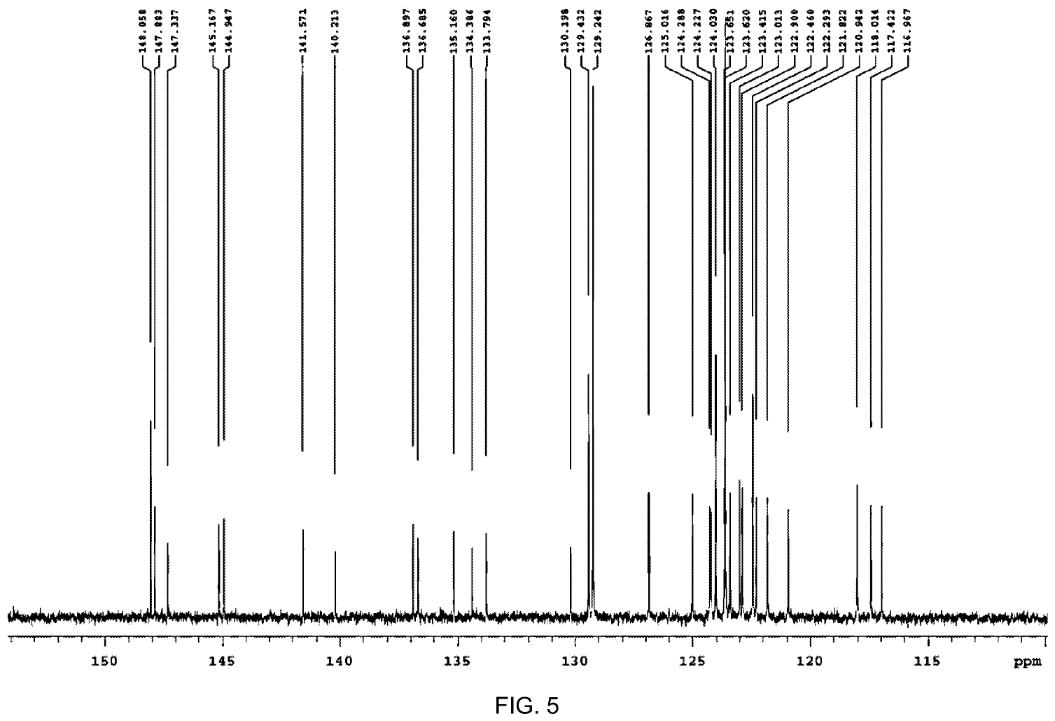

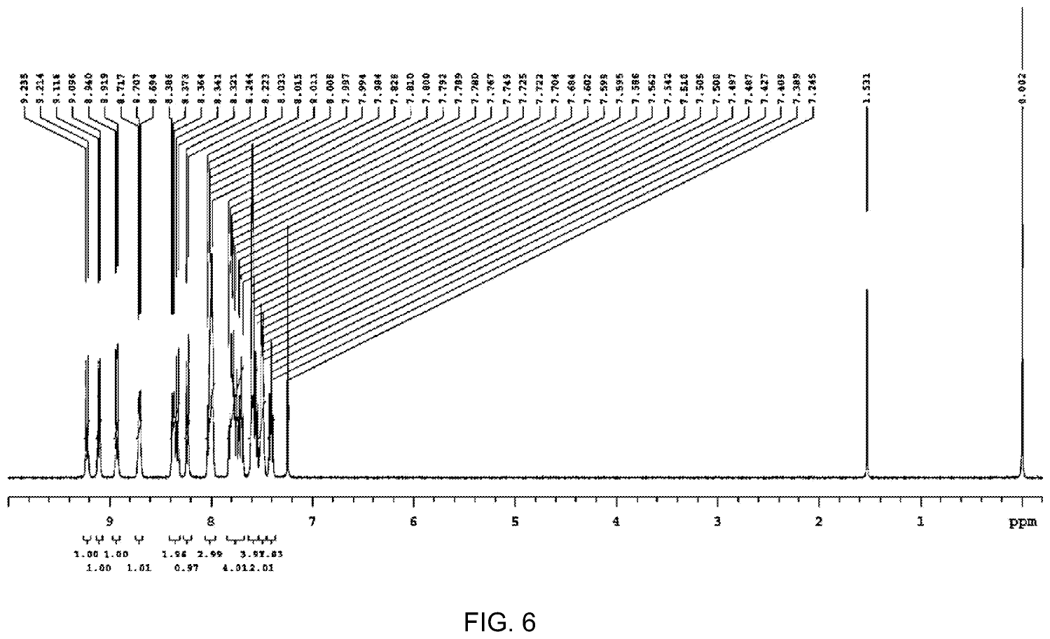

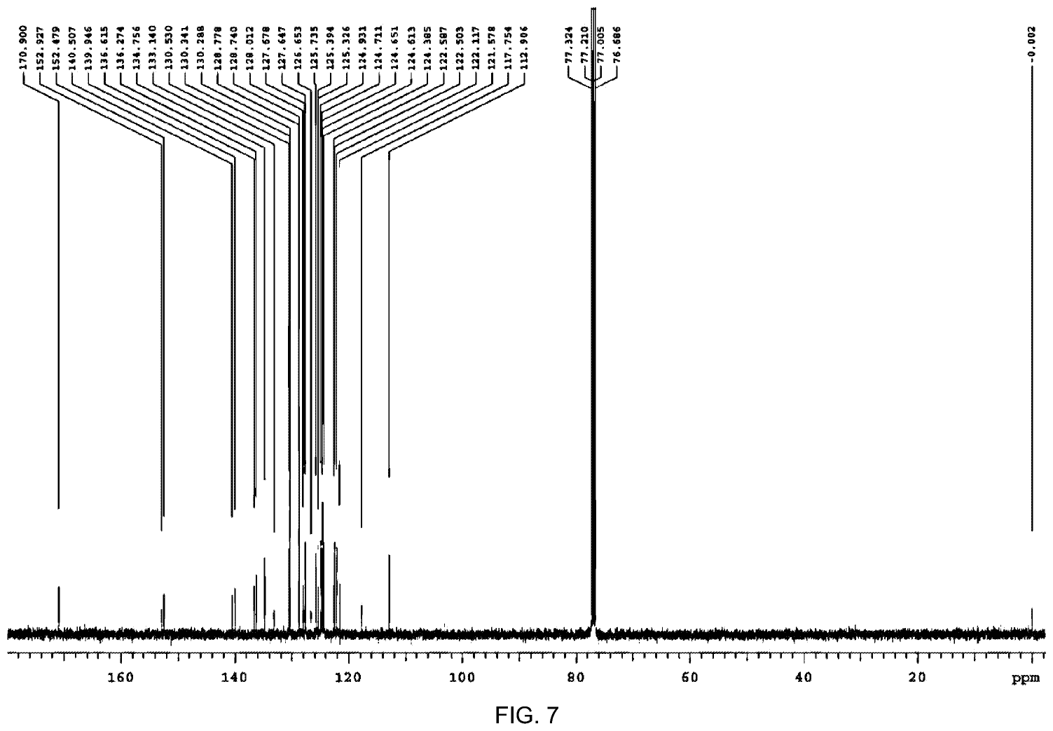

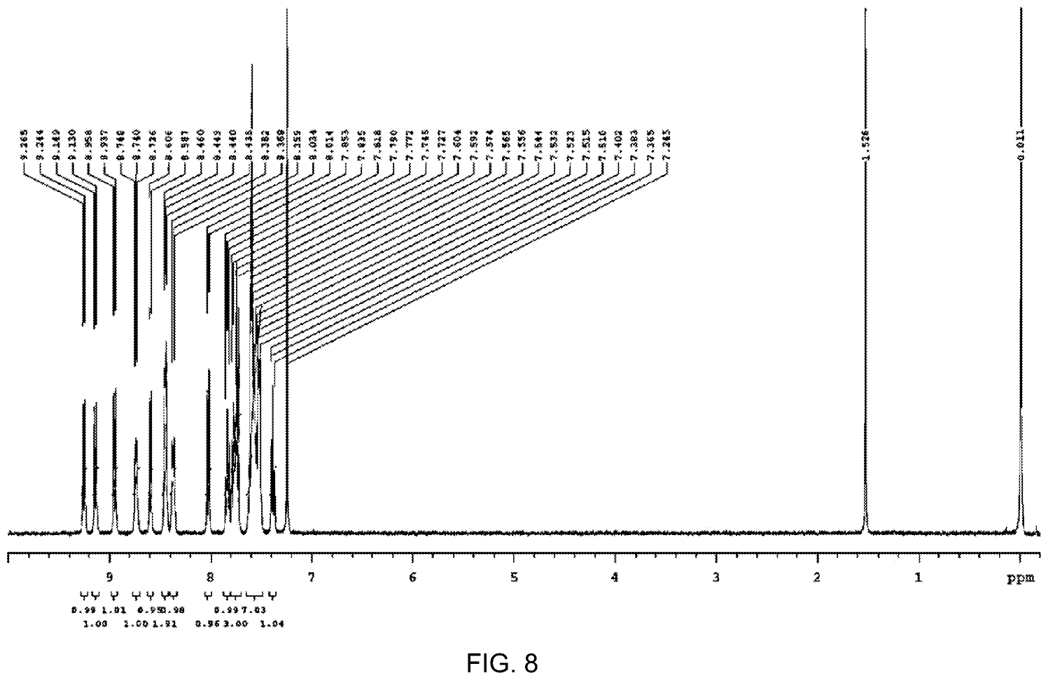

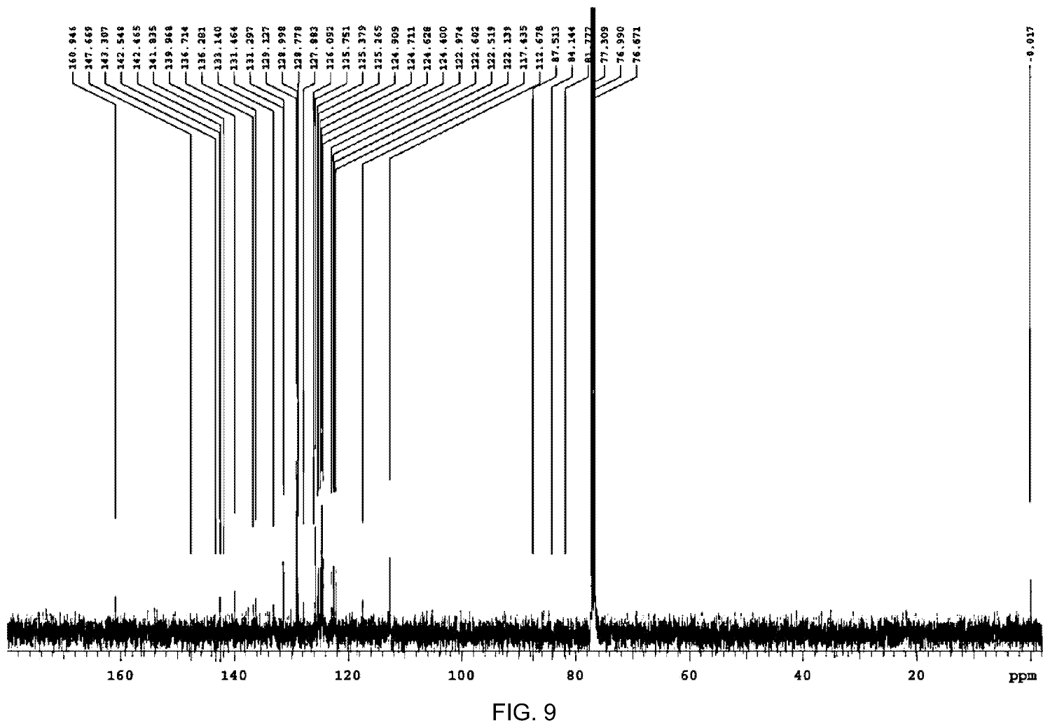

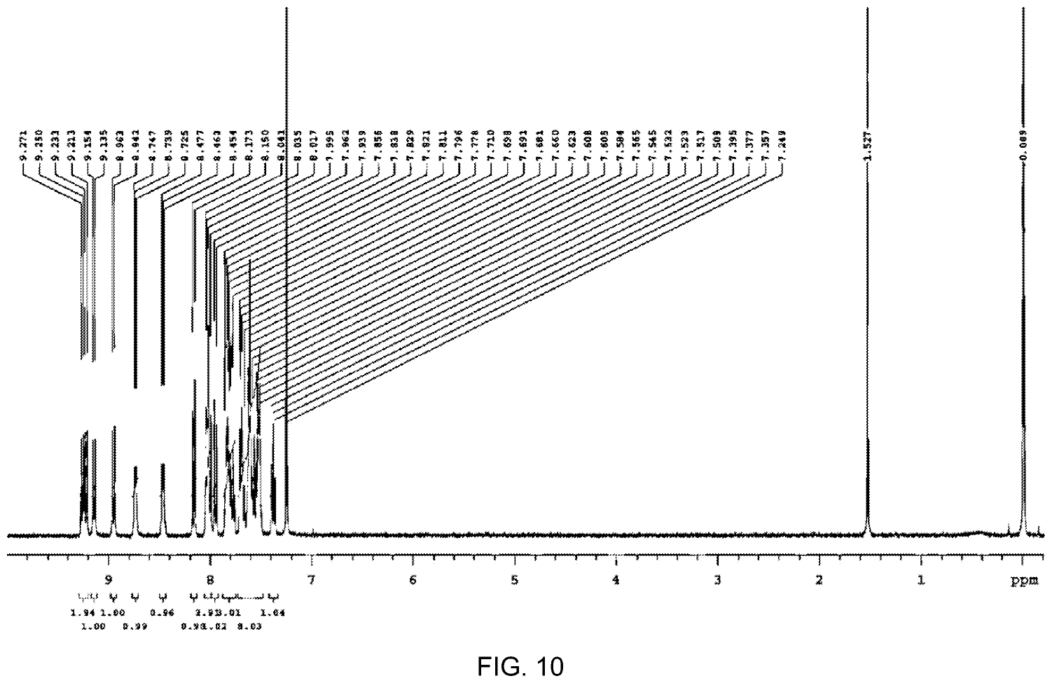

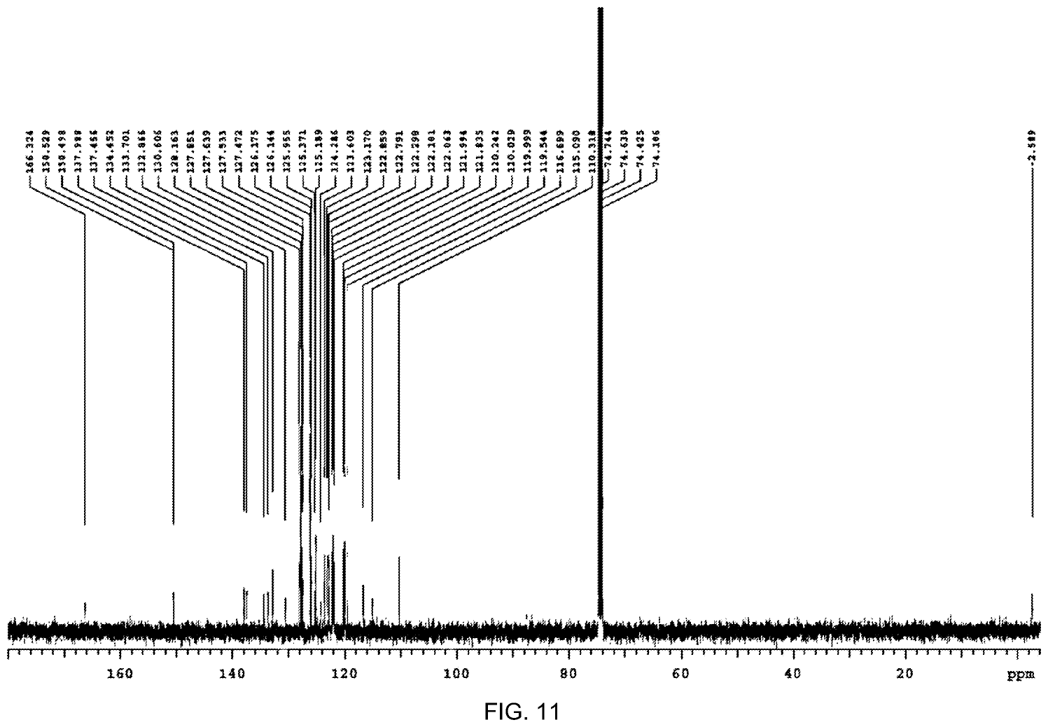

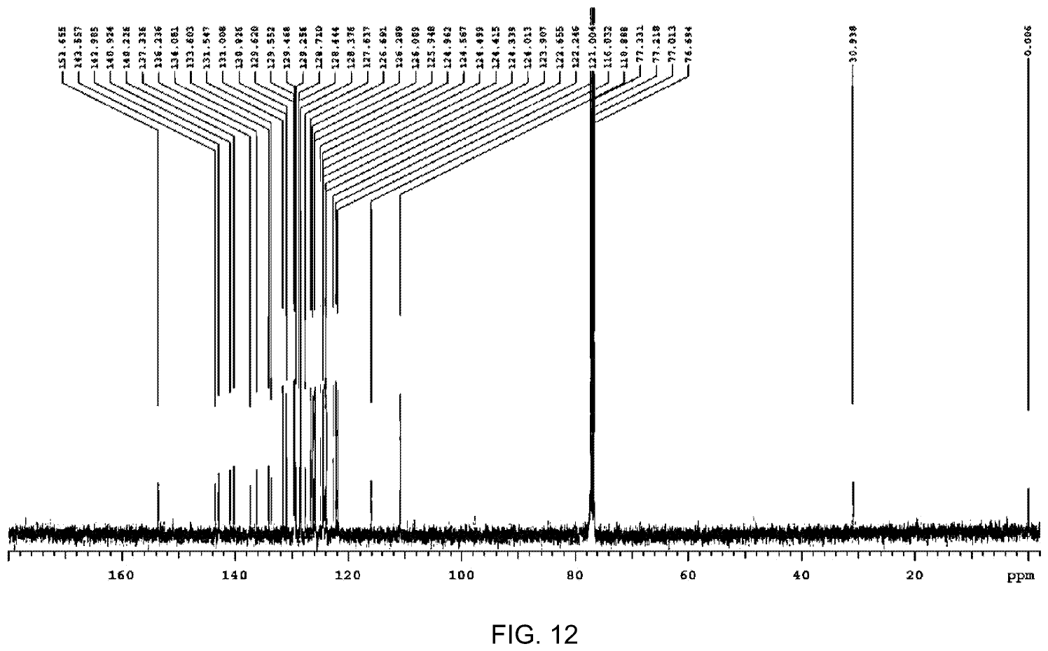

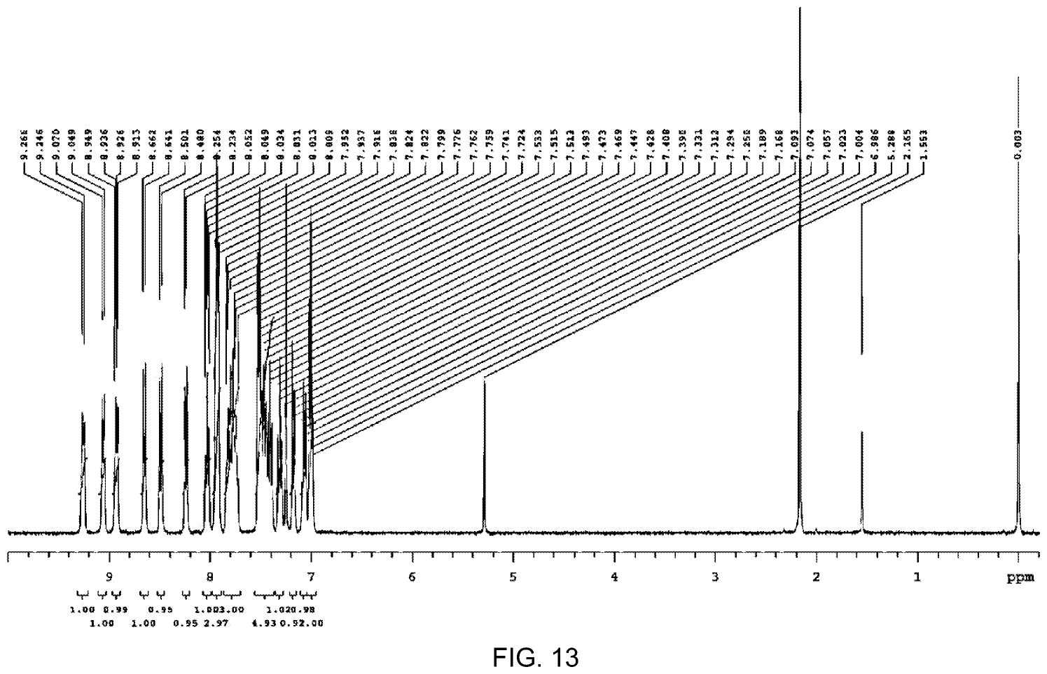

Referring to FIG. 1, the organic electric element (100) according to the present invention includes a first electrode (120) formed on a substrate (110), a second electrode (180), and an organic material layer including the compound represented by Formula (1) between the first electrode (120) and the second electrode (180). Here, the first electrode (120) may be an anode (positive electrode), and the second electrode (180) may be a cathode (negative electrode). In the case of an inverted organic electric element, the first electrode may be a cathode, and the second electrode may be an anode.

The organic material layer may include a hole injection layer (130), a hole transport layer (140), an emitting layer (150), an electron transport layer (160), and an electron injection layer (170) formed in sequence on the first electrode (120). Here, the remaining layers except the emitting layer (150) may not be formed. The organic material layer may further include a hole blocking layer, an electron blocking layer, an emitting-auxiliary layer (151), an electron transport auxiliary layer, a buffer layer (141), etc., and the electron transport layer (160) and the like may serve as a hole blocking layer.

Although not shown, the organic electric element according to the present invention may further include a protective layer formed on at least one side of the first and second electrodes, which is a side opposite to the organic material layer.

Otherwise, even if the same core is used, the band gap, the electrical characteristics, the interface characteristics, and the like may vary depending on which substituent is bonded at which position, therefore the choice of core and the combination of sub-substituents associated therewith is also very important, and in particular, when the optimal combination of energy levels and T1 values and unique properties of materials (mobility, interfacial characteristics, etc.) of each organic material layer is achieved, a long life span and high efficiency can be achieved at the same time.

The organic electroluminescent device according to an embodiment of the present invention may be manufactured using a PVD (physical vapor deposition) method. For example, a metal or a metal oxide having conductivity or an alloy thereof is deposited on a substrate to form a cathode, and the organic material layer including the hole injection layer (130), the hole transport layer (140), the emitting layer (150), the electron transport layer (160), and the electron injection layer (170) is formed thereon, and then depositing a material usable as a cathode thereon can manufacture an organic electroluminescent device according to an embodiment of the present invention.

In addition, an emission auxiliary layer (151) may be further formed between the hole transport layer (140) and the emitting layer (150), and an electron transport auxiliary layer may be further formed between the emitting layer (150) and the electron transport layer (160).

In addition, at least one hole transporting band layer is provided between the first electrode and the emitting layer, wherein the hole transporting band layer may include a hole transport layer, an emitting auxiliary layer or both, wherein the hole transporting band layer includes an organic electronic element comprising the compound represented by Formula (1).

The present invention may further include a light efficiency enhancing layer formed on at least one of the opposite side to the organic material layer among one side of the first electrode, or one of the opposite side to the organic material layer among one side of the second electrode.

Also, the present invention provides the organic electric element wherein the organic material layer is formed by one of a spin coating process, a nozzle printing process, an inkjet printing process, a slot coating process, a dip coating process or a roll-to-roll process, and since the organic material layer according to the present invention can be formed by various methods, the scope of the present invention is not limited by the method of forming the organic material layer.

As another specific example, the present invention provides an organic electric element wherein the emitting layer in the organic material layer is a phosphorescent light emitting layer.

The compounds represented by Formula (1) and (2) are mixed in a ratio of any one of 1:9 to 9:1 to be included in the emitting layer of the organic material layer.

The compound represented by Formula (1) and (2) are mixed in a ratio of any one of 1:9 to 5:5 to be included in the emitting. Preferably, the mixing ratio of the compound represented by Formula (1) and the compound represented by Formula (2) is mixed at a ratio of 1:9 or 5:5, to be included as the emitting layer, or the mixture ratio is mixed in a ratio of 2:8 to 3:7, to be used in the emitting layer. More preferably, the mixing ratio of the compound represented by Formula (1) and the compound represented by Formula (2) is mixed at a ratio of 2:8 or 3:7, to be included in the emitting layer.

In another aspect, in one embodiment of the present invention, the present invention provides a first electrode; a second electrode; and an organic material layer disposed between the first electrode and the second electrode and including at least an hole transport layer, an emitting auxiliary layer and an emitting layer, wherein the hole transport layer or the emitting auxiliary layer comprise a compound represented by Formula (1), wherein the emitting layer comprises a compound represented by Formula (2). That is, the compound represented by Formula (1) can be used as the material of the hole transport layer and/or the emitting auxiliary layer.

In another aspect, in one embodiment of the present invention, the present invention provides a first electrode; a second electrode; and an organic material layer disposed between the first electrode and the second electrode and including at least an emitting auxiliary layer and an emitting layer, wherein at least one emitting auxiliary layer material of the organic material layers comprises a compound represented by Formula (1), wherein at least one host material in the emitting layer comprises a compound represented by Formula (2).

The organic electric element according to an embodiment of the present invention may be a front emission type, a back emission type, or a both-sided emission type, depending on the material used.

WOLED (White Organic Light Emitting Device) has advantages of high resolution realization and excellent fairness, and can be manufactured using conventional LCD color filter technology. Various structures for a white organic light emitting device mainly used as a backlight device have been proposed and patented. Representatively, there are side-by-side arrangement of the radiation part of the R (red), G (green) and B (blue), a stacking method in which R, G, and B emitting layers are laminated on top and bottom, electroluminescence by the blue (B) organic emitting layer and, by using the light from this, a color conversion material (CCM) method using a photo-luminescence of an inorganic phosphor, etc., and the present invention may be applied to such WOLED.

The present invention also provides an electronic device comprising a display device including the organic electric element; and a control unit for driving the display device.

According to another aspect, the present invention provides an display device wherein the organic electric element is at least one of an OLED, an organic solar cell, an organic photo conductor, an organic transistor (organic TFT) and an element for monochromic or white illumination. Here, the electronic device may be a wired/wireless communication terminal which is currently used or will be used in the future, and covers all kinds of electronic devices including a mobile communication terminal such as a cellular phone, a personal digital assistant (PDA), an electronic dictionary, a point-to-multipoint (PMP), a remote controller, a navigation unit, a game player, various kinds of TVs, and various kinds of computers.

Hereinafter, Synthesis Examples of the compound represented by Formula (1) and (2) of the present invention and preparation examples of the organic electric element of the present invention will be described in detail by way of example, but are not limited to the following examples.

### Synthesis Example 1

### I. Synthesis of Formula (1)

The final products 1 represented by Formula (1) of the present invention can be synthesized by reaction between Sub 1 and Sub 2 as illustrated in the following Reaction Scheme 1 or Reaction Scheme 2.

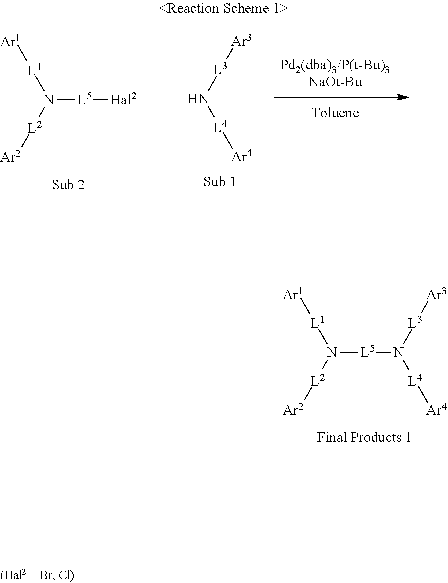

When L⁵ of the Final Products of Reaction Scheme 1 is

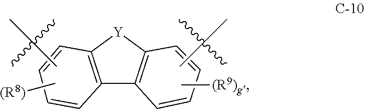

Final Products 1′ can be synthesized by the following reaction path, but not limited thereto.

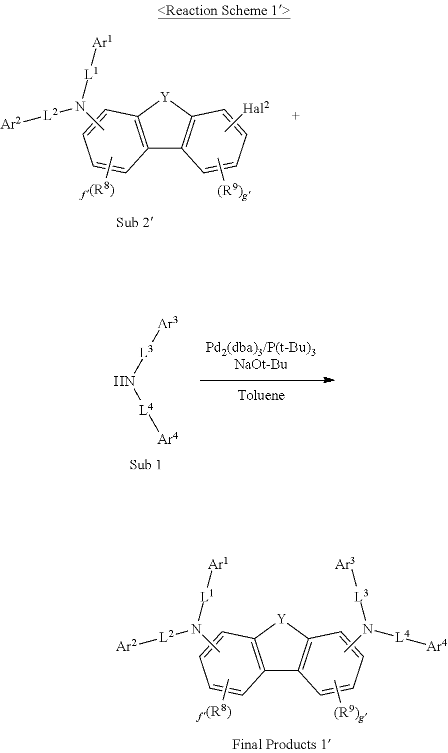

Ar¹, Ar², Ar³, Ar⁴, L¹, L², L³, L⁴, Y, R⁸, R⁹, Land g′ are the same as defined above.

In the reaction scheme 1 ‘, the synthesis method disclosed in Korean Patent No. 10-1668448 filed by the present applicant was used in the case of Final Products 1’. (See Reaction Scheme 1)

<Reaction Scheme 2> L¹ and L² are single bonds, and Ar¹ and Ar² form a ring.

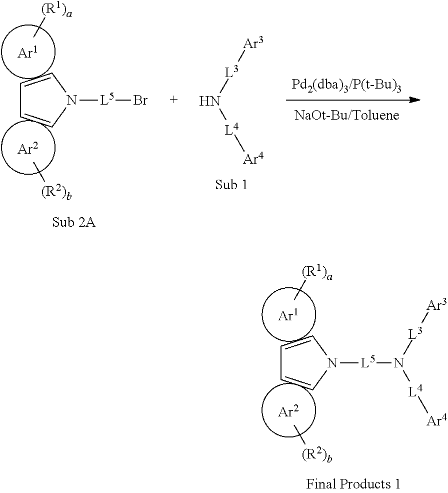

### 1. Synthesis Example of Sub 1

Sub 1 of reaction scheme 1 can be synthesized by the reaction path of the following reaction scheme 3, but is not limited thereto.

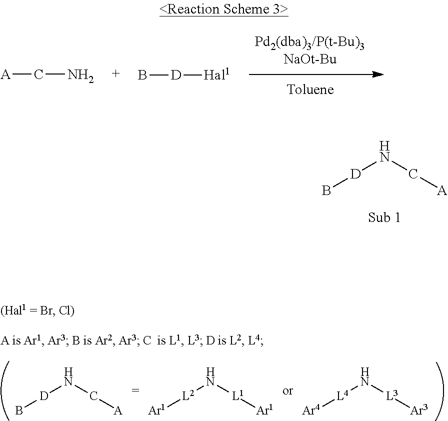

The synthesis examples of specific compounds belonging to Sub 1 are as follows.

### (1) Synthesis of Sub 1-1

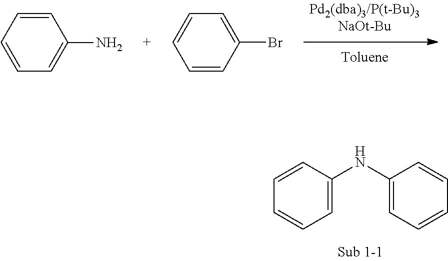 [SMILES: Nc1ccccc1 ; Brc1ccccc1 ; c1ccc(Nc2ccccc2)cc1]

Aniline (40 g, 429.5 mmol) was dissolved in toluene (3000 ml) in a round bottom flask, and bromobenzene (74.18 g, 472.5 mmol), Pd₂(dba)₃ (19.66 g, 21.5 mmol), 50% P(t-Bu)₃ (20.9 ml, 43 mmol), NaOt-Bu (136.22 g, 1417.4 mmol) were added and stirred at 100° C. After the reaction was completed, the reaction mixture was extracted with CH₂Cl₂ and water. The organic layer was dried over MgSO₄ and concentrated. The resulting compound was separated by silicagel column chromatography and recrystallized to obtain 54.51 g of the product. (Yield: 75%)

### (2) Synthesis of Sub 1-11

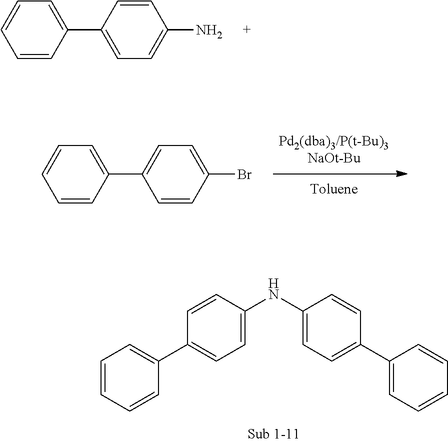 [SMILES: Nc1ccc(-c2ccccc2)cc1 ; Brc1ccc(-c2ccccc2)cc1 ; c1ccc(-c2ccc(Nc3ccc(-c4ccccc4)cc3)cc2)cc1]

[1,1′-biphenyl]-4-amine (30 g, 177.3 mmol), 4-bromo-1,1′-biphenyl (45.46 g, 195 mmol), Pd₂(dba)₃ (8.12 g, 8.9 mmol), 50% P(t-Bu)₃ (8.6 ml, 17.7 mmol), NaOt-Bu (56.23 g, 585 mmol), toluene (1860 ml) were carried out in the same manner as in Sub 1-1 to give the product (45.01 g, 79%).

### (3) Synthesis of Sub 1-22

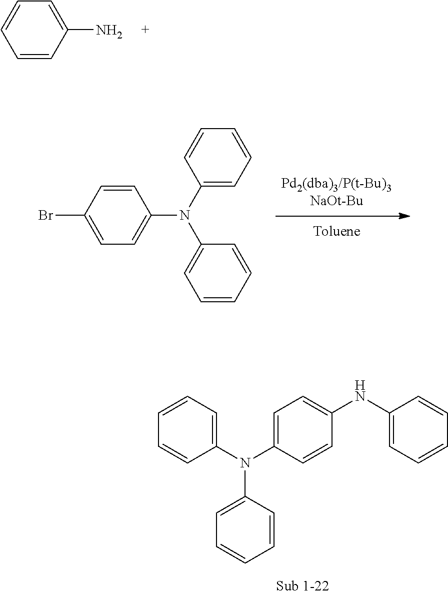 [SMILES: Nc1ccccc1 ; Brc1ccc(N(c2ccccc2)c2ccccc2)cc1 ; c1ccc(Nc2ccc(N(c3ccccc3)c3ccccc3)cc2)cc1]

aniline (12.12 g, 130.16 mmol), 4-bromo-N,N-diphenylaniline (42.2 g 130.16 mmol), Pd₂(dba)₃ (3.58 g, 3.90 mmol), P(t-Bu)₃ (1.58 g, 7.81 mmol), NaOt-Bu (37.52 g, 390.48 mmol), toluene (1367 ml) were carried out in the same manner as in Sub 1-1 to give the product (34.16 g, 78%).

### (4) Synthesis of Sub 1-40

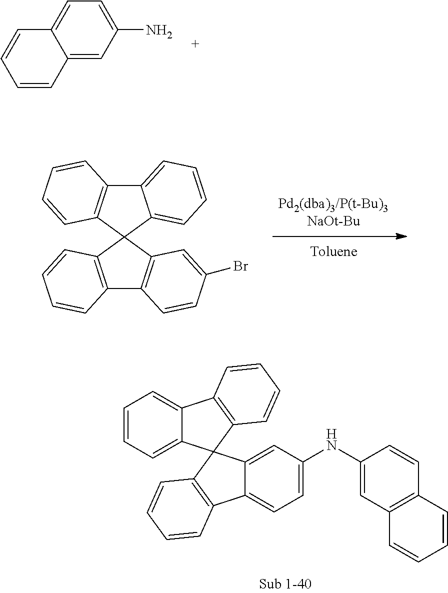 [SMILES: Nc1ccc2ccccc2c1 ; Brc1ccc2c(c1)C1(c3ccccc3-c3ccccc31)c1ccccc1-2 ; c1ccc2c(c1)-c1ccccc1C21c2ccccc2-c2ccc(Nc3ccc4ccccc4c3)cc21]

naphthalen-2-amine (14.85 g, 103.72 mmol), 2-bromo-9,9′-spirobi[fluorene] (41 g, 103.72 mmol), Pd₂(dba)₃ (2.85 g, 3.11 mmol), P(t-Bu)₃ (1.26 g, 6.22 mmol), NaOt-Bu (29.90 g, 311.16 mmol), toluene (1089 ml) were carried out in the same manner as in Sub 1-1 to give the product (34.17 g, 72%).

### (5) Synthesis of Sub 1-46

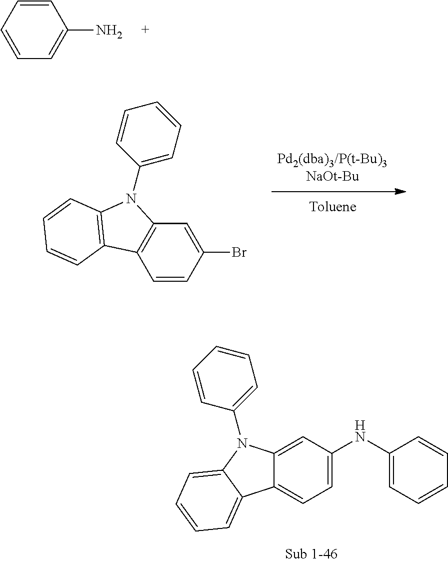 [SMILES: Nc1ccccc1 ; Brc1ccc2c3ccccc3n(-c3ccccc3)c2c1 ; c1ccc(Nc2ccc3c4ccccc4n(-c4ccccc4)c3c2)cc1]

aniline (15 g, 161.1 mmol), 2-bromo-9-phenyl-9H-carbazole (57.08 g, 177.2 mmol), Pd₂(dba)₃ (7.37 g, 8.1 mmol), 50% P(t-Bu)₃ (7.9 ml, 16.1 mmol), NaOt-Bu (51.08 g, 531.5 mmol), toluene (1690 ml) were carried out in the same manner as in Sub 1-1 to give the product (36.63 g, 68%).

### (6) Synthesis of Sub 1-57

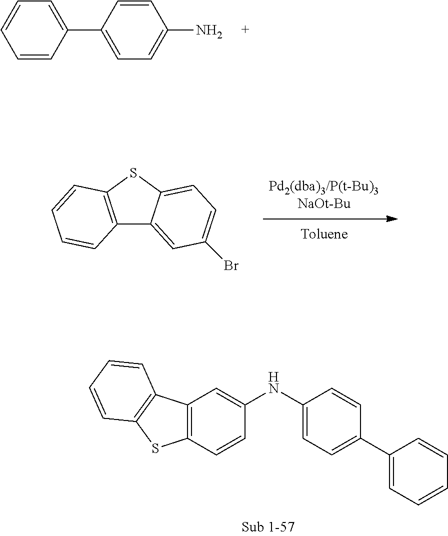 [SMILES: Nc1ccc(-c2ccccc2)cc1 ; Brc1ccc2sc3ccccc3c2c1 ; c1ccc(-c2ccc(Nc3ccc4sc5ccccc5c4c3)cc2)cc1]

[1,1′-biphenyl]-4-amine (22.51 g, 133 mmol), 2-bromodibenzo[b,d]thiophene (35 g, 133 mmol), Pd₂(dba)₃ (3.65 g, 3.99 mmol), P(t-Bu)₃ (3.65 g, 3.99 mmol), NaOt-Bu (38.35 g, 399.01 mmol), toluene (1397 ml) were carried out in the same manner as in Sub 1-1 to give the product (34.59 g, 74%).

### (7) Synthesis of Sub 1-69

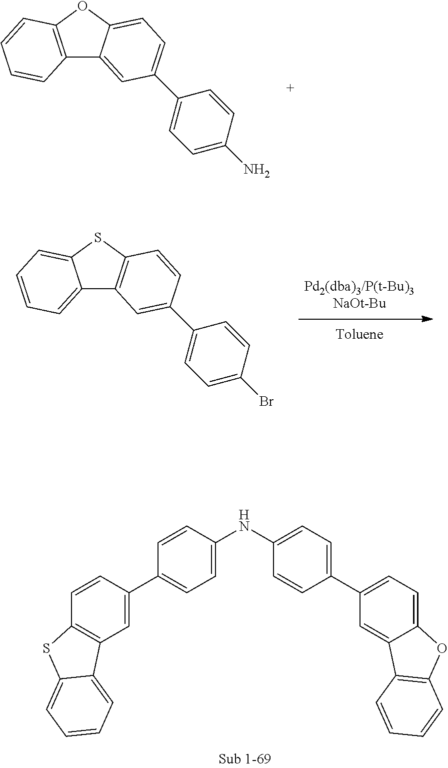 [SMILES: Nc1ccc(-c2ccc3oc4ccccc4c3c2)cc1 ; Brc1ccc(-c2ccc3sc4ccccc4c3c2)cc1 ; c1ccc2c(c1)oc1ccc(-c3ccc(Nc4ccc(-c5ccc6sc7ccccc7c6c5)cc4)cc3)cc12]

4-(dibenzo[b,d]furan-2-yl)aniline (24.46 g, 94.33 mmol), 2-(4-bromophenyl)dibenzo[b,d]thiophene (32 g, 94.33 mmol), Pd₂(dba)₃ (2.59 g, 2.83 mmol), P(t-Bu)₃ (1.15 g, 5.66 mmol), NaOt-Bu (27.19 g, 282.98 mmol), toluene (990 ml) were carried out in the same manner as in Sub 1-1 to give the product (34.18 g, 70%).

### (8) Synthesis of Sub 1-93

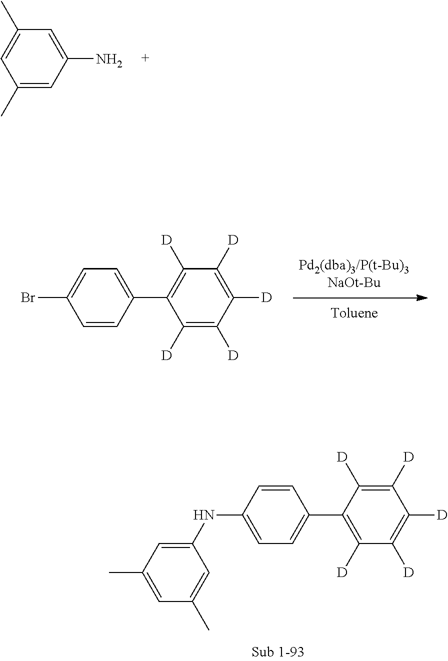

3,5-dimethylaniline (21.88 g, 180.57 mmol), 4-bromo-1,1′-biphenyl-2′,3′,4′,5′,6′-d₅ (43 g, 180.57 mmol), Pd₂(dba)₃ (4.96 g, 5.42 mmol), P(t-Bu)₃ (2.19 g, 10.83 mmol), NaOt-Bu (52.06 g, 541.70 mmol), toluene (1896 ml) were carried out in the same manner as in Sub 1-1 to give the product (34.18 g, 68%).

Examples of Sub 1 are as follows, but are not limited thereto

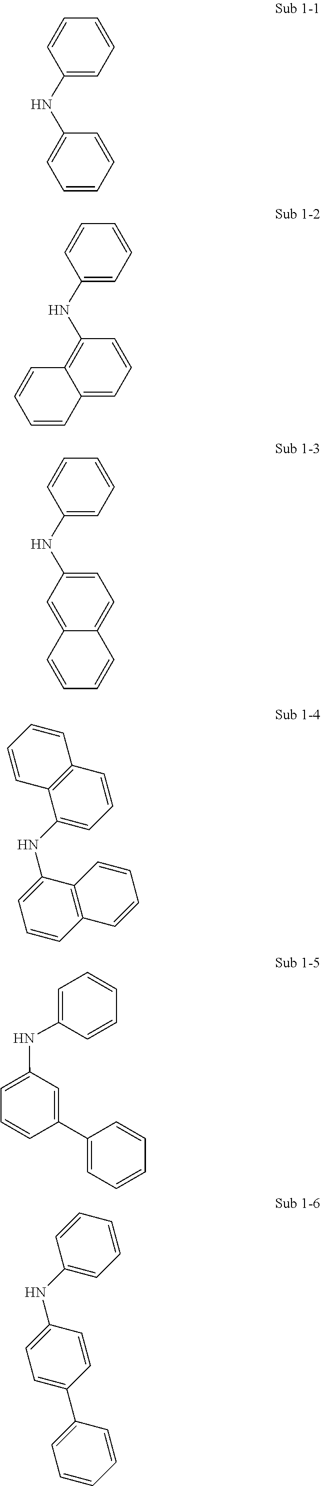

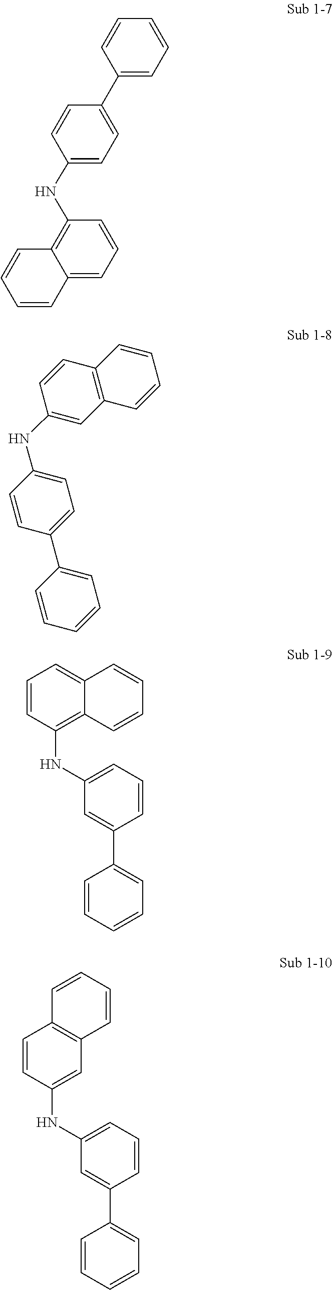

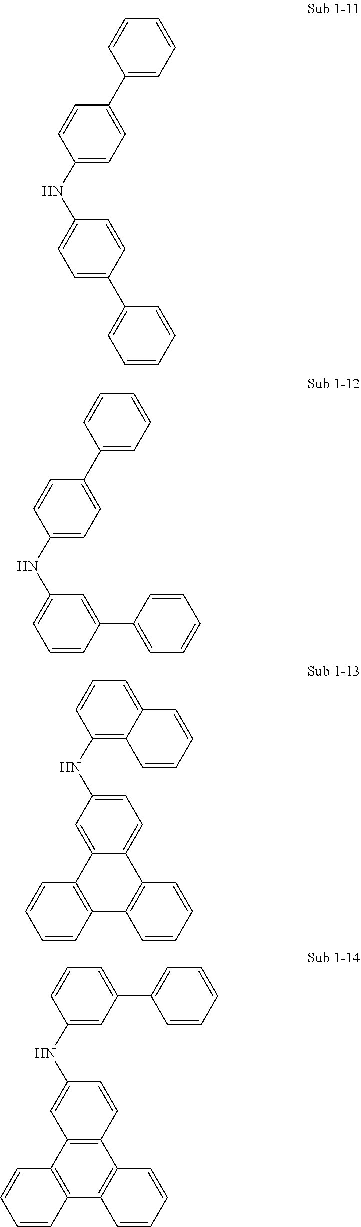

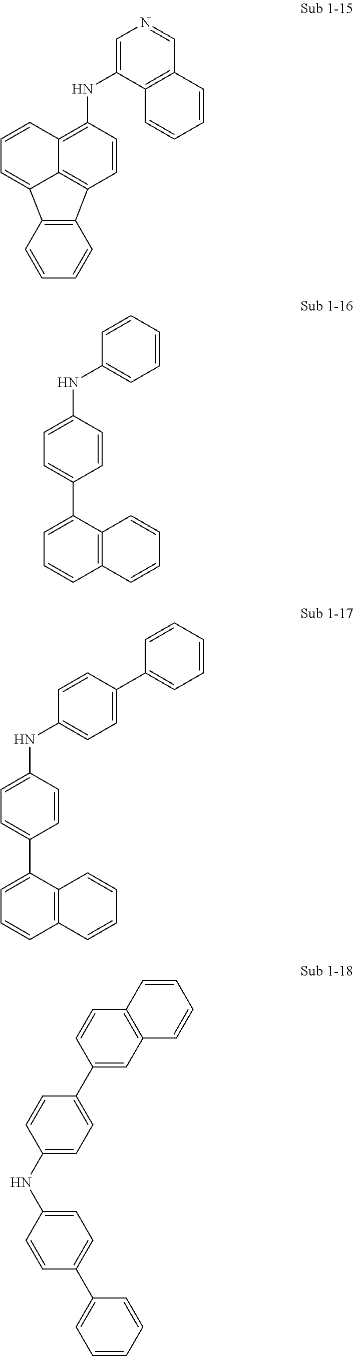 [SMILES: c1ccc2c(c1)-c1cccc3c(Nc4cncc5ccccc45)ccc-2c13 ; c1ccc(Nc2ccc(-c3cccc4ccccc34)cc2)cc1 ; c1ccc(-c2ccc(Nc3ccc(-c4cccc5ccccc45)cc3)cc2)cc1 ; c1ccc(-c2ccc(Nc3ccc(-c4ccc5ccccc5c4)cc3)cc2)cc1]

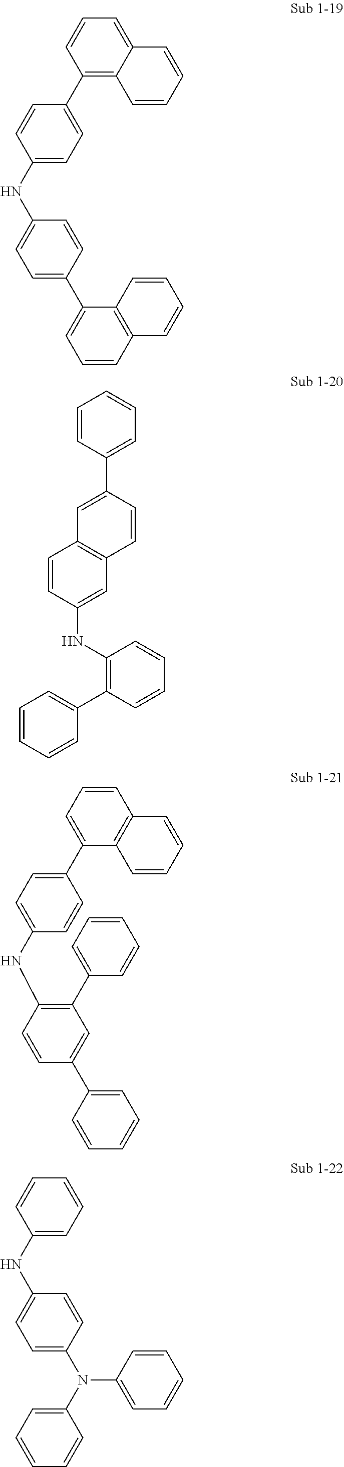

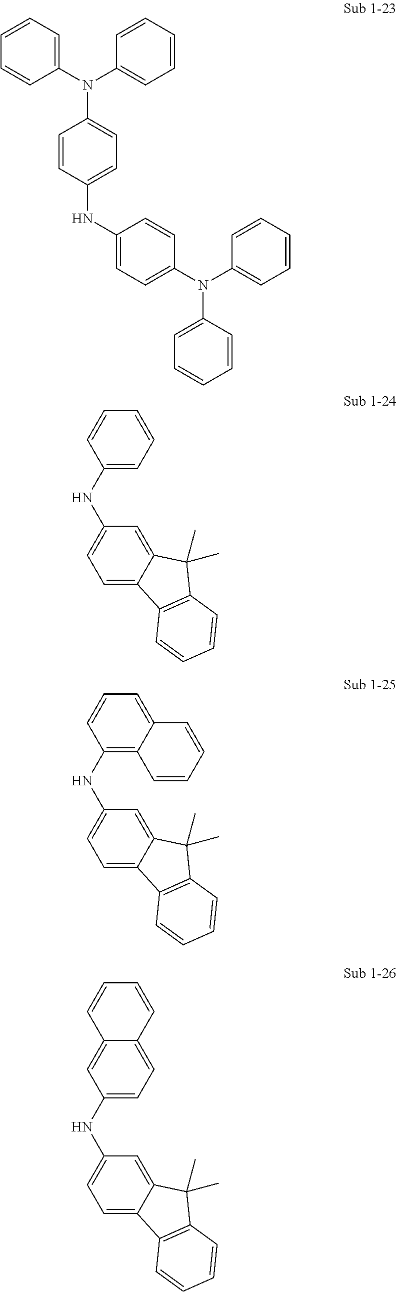 [SMILES: c1ccc(N(c2ccccc2)c2ccc(Nc3ccc(N(c4ccccc4)c4ccccc4)cc3)cc2)cc1 ; CC1(C)c2ccccc2-c2ccc(Nc3ccccc3)cc21 ; CC1(C)c2ccccc2-c2ccc(Nc3cccc4ccccc34)cc21 ; CC1(C)c2ccccc2-c2ccc(Nc3ccc4ccccc4c3)cc21]

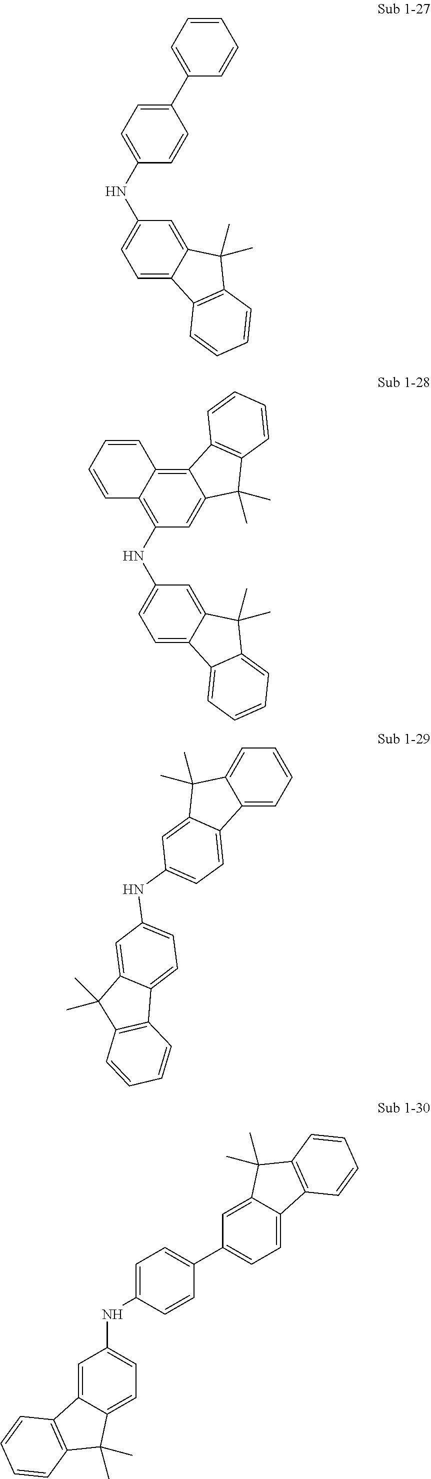

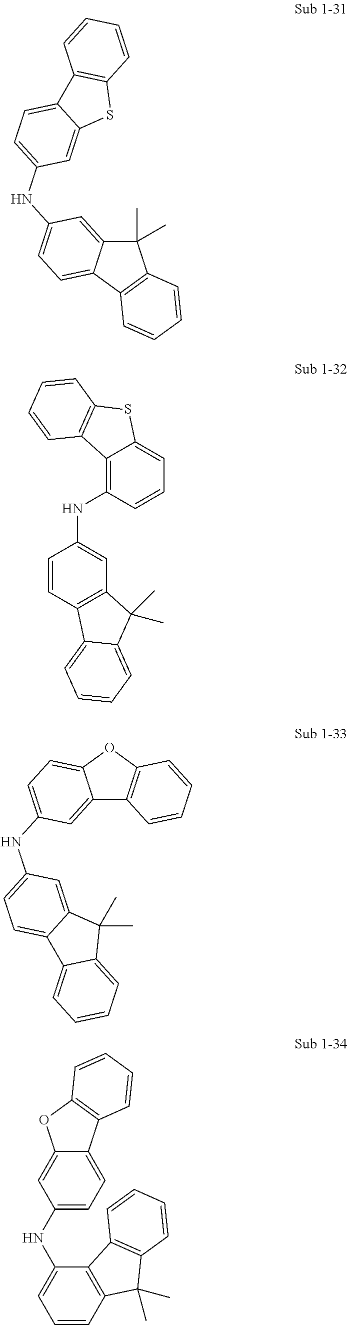

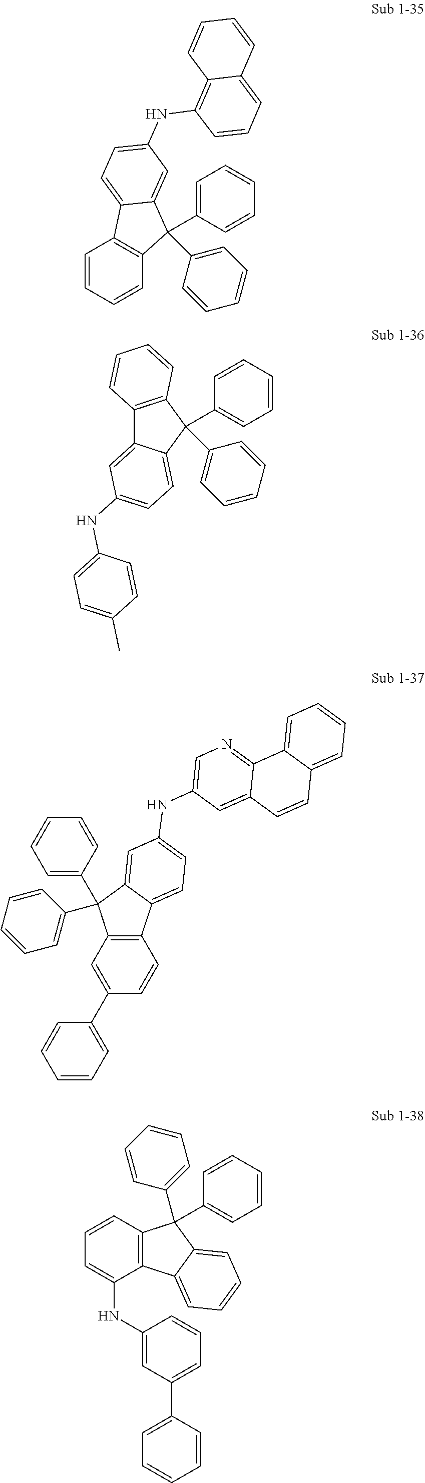 [SMILES: c1ccc(C2(c3ccccc3)c3ccccc3-c3ccc(Nc4cccc5ccccc45)cc32)cc1 ; Cc1ccc(Nc2ccc3c(c2)-c2ccccc2C3(c2ccccc2)c2ccccc2)cc1 ; c1ccc(-c2ccc3c(c2)C(c2ccccc2)(c2ccccc2)c2cc(Nc4cnc5c(ccc6ccccc65)c4)ccc2-3)cc1 ; c1ccc(-c2cccc(Nc3cccc4c3-c3ccccc3C4(c3ccccc3)c3ccccc3)c2)cc1]

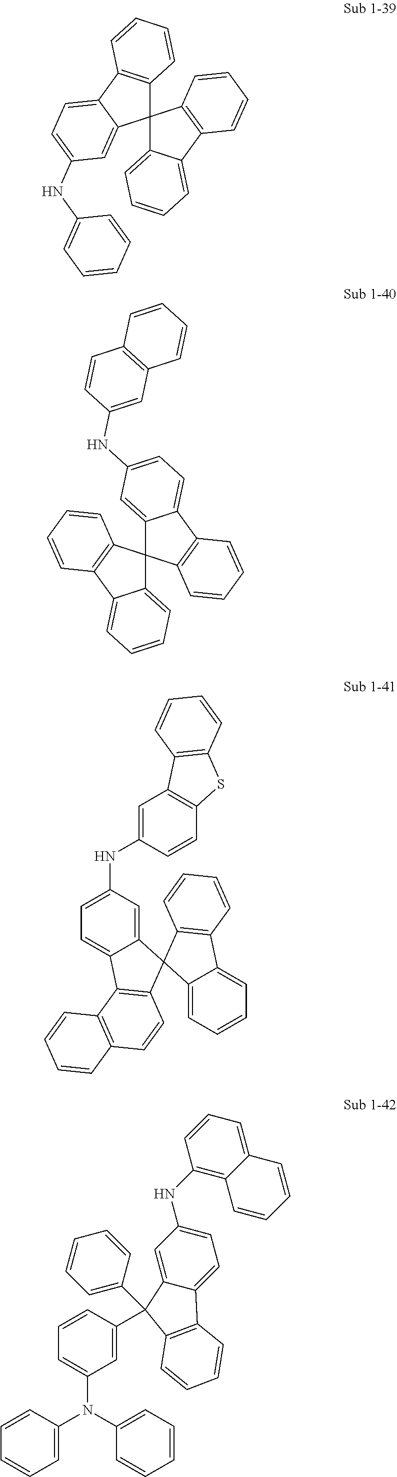

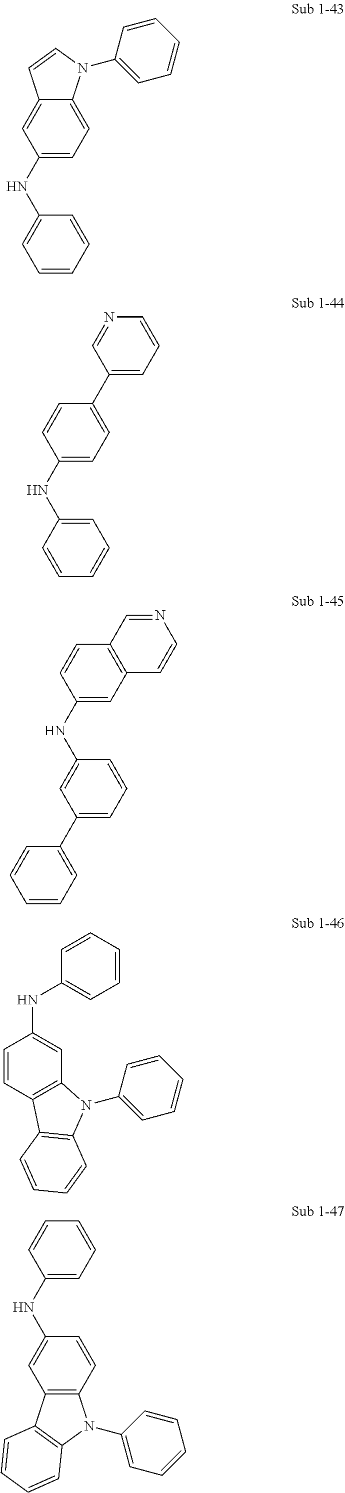

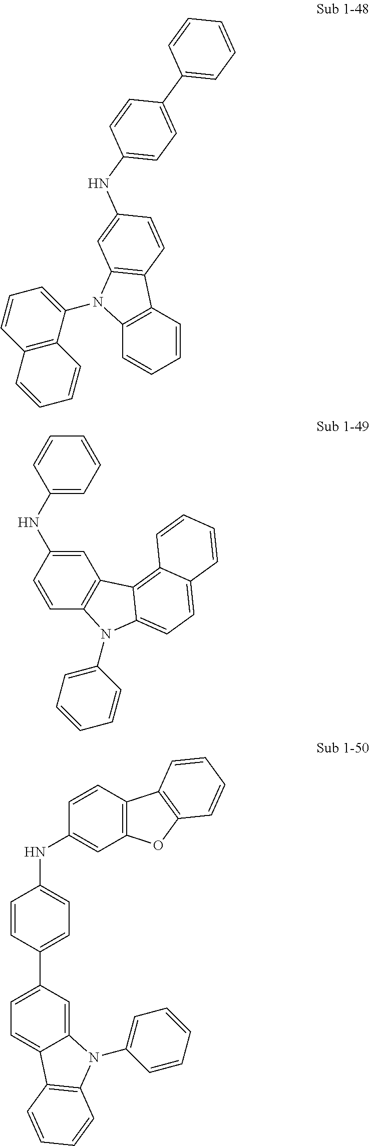

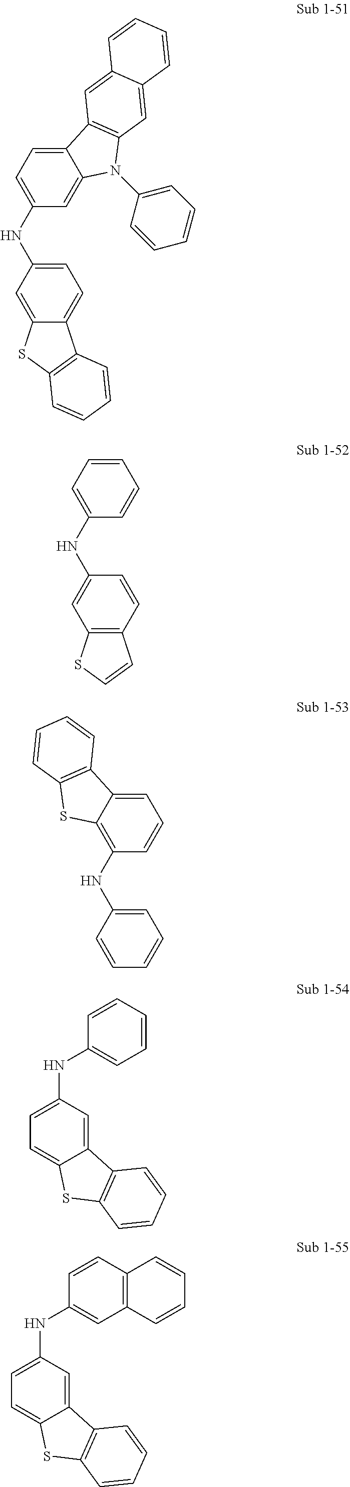

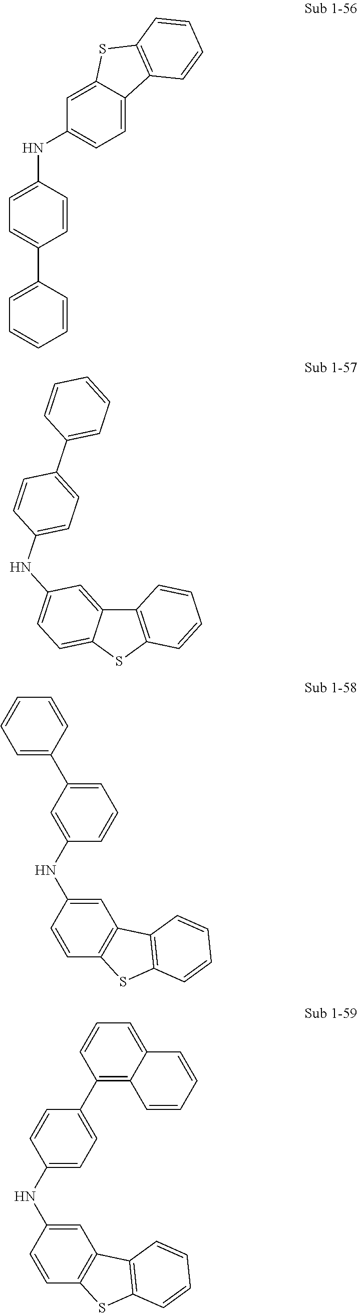

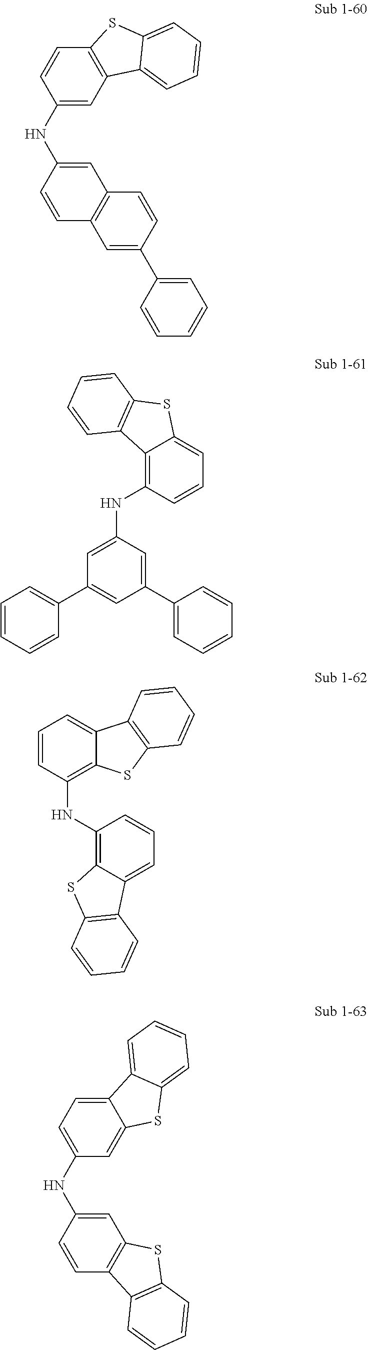

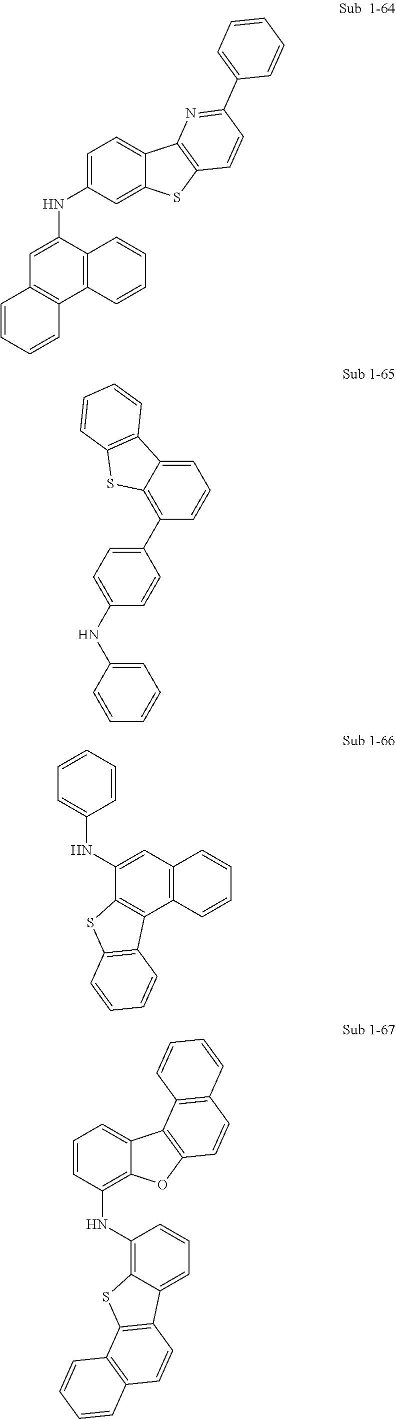

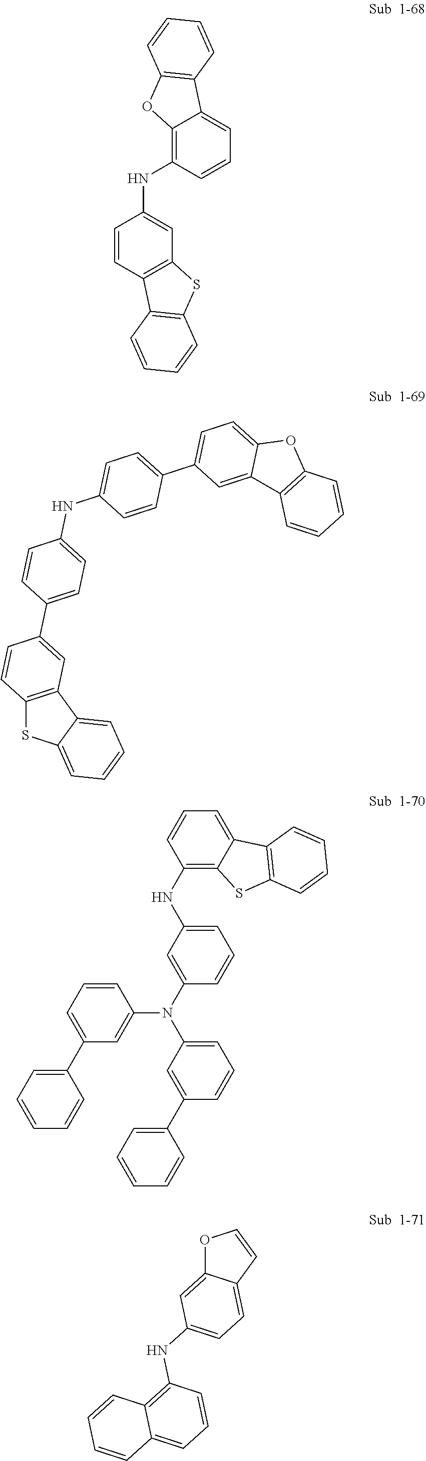 [SMILES: c1ccc2c(c1)oc1c(Nc3ccc4c(c3)sc3ccccc34)cccc12 ; c1ccc2c(c1)oc1ccc(-c3ccc(Nc4ccc(-c5ccc6sc7ccccc7c6c5)cc4)cc3)cc12 ; c1ccc(-c2cccc(N(c3cccc(Nc4cccc5c4sc4ccccc45)c3)c3cccc(-c4ccccc4)c3)c2)cc1 ; c1ccc2c(Nc3ccc4ccoc4c3)cccc2c1]

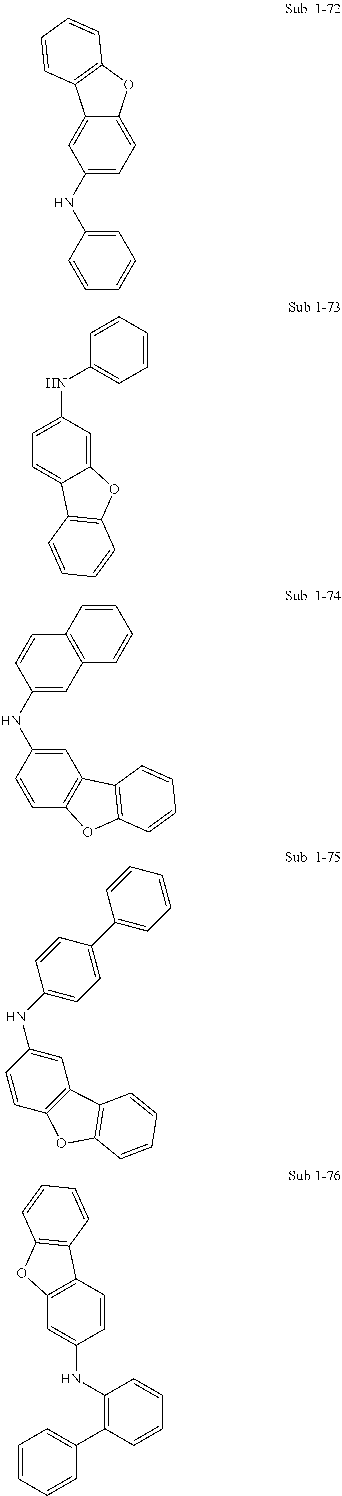

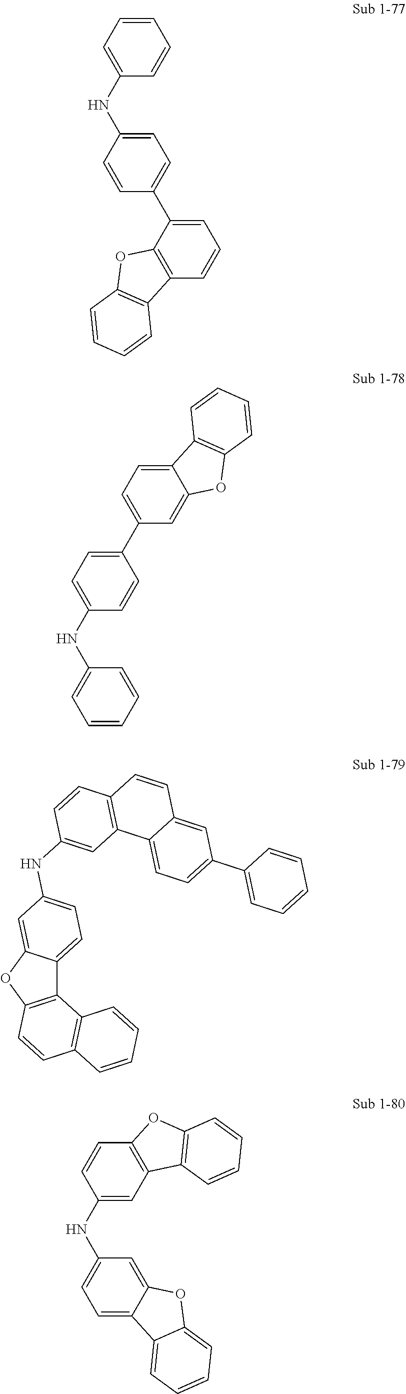 [SMILES: c1ccc(Nc2ccc(-c3cccc4c3oc3ccccc34)cc2)cc1 ; c1ccc(Nc2ccc(-c3ccc4c(c3)oc3ccccc34)cc2)cc1 ; c1ccc(-c2ccc3c(ccc4ccc(Nc5ccc6c(c5)oc5ccc7ccccc7c56)cc43)c2)cc1 ; c1ccc2c(c1)oc1cc(Nc3ccc4oc5ccccc5c4c3)ccc12]

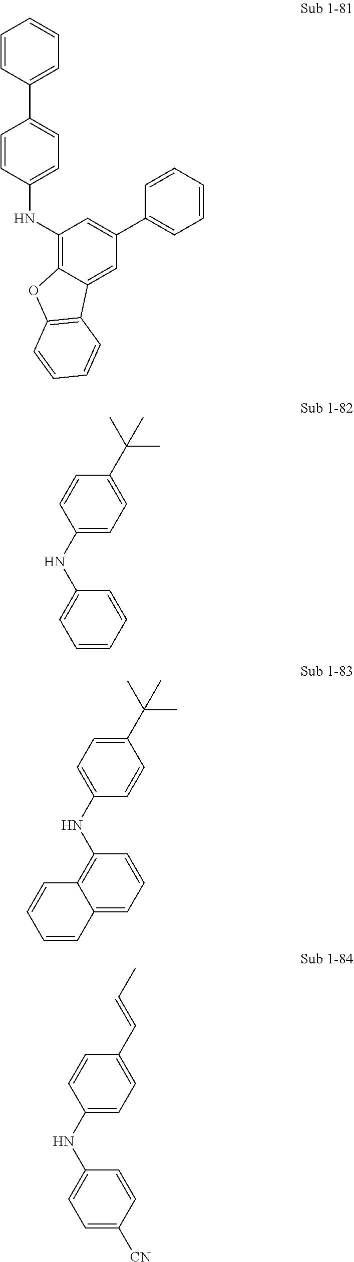

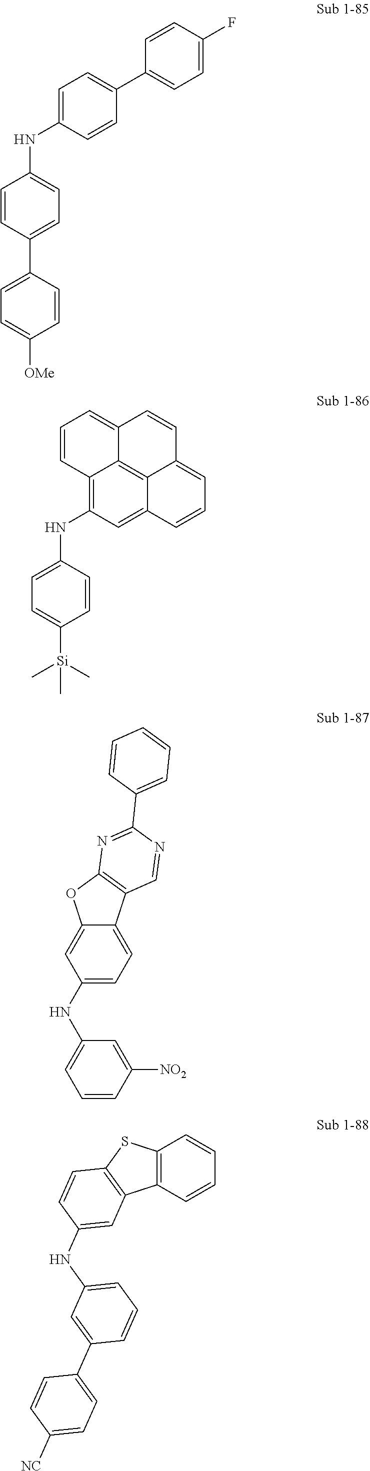

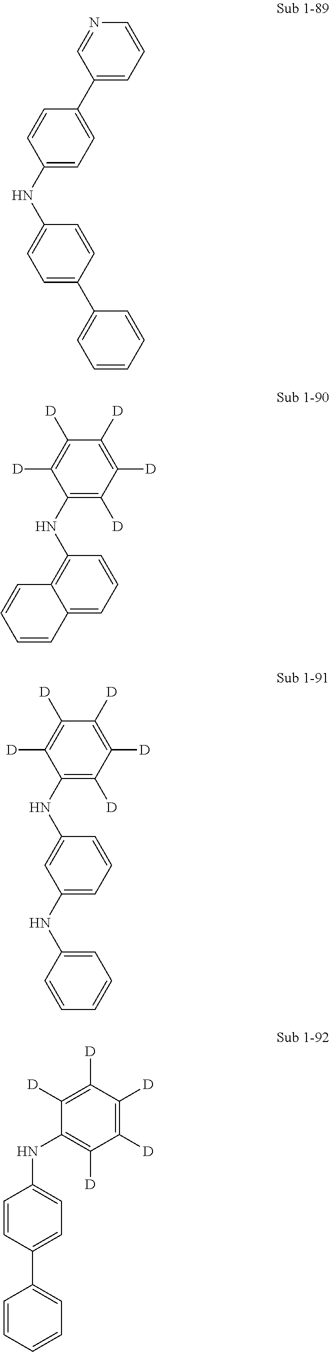

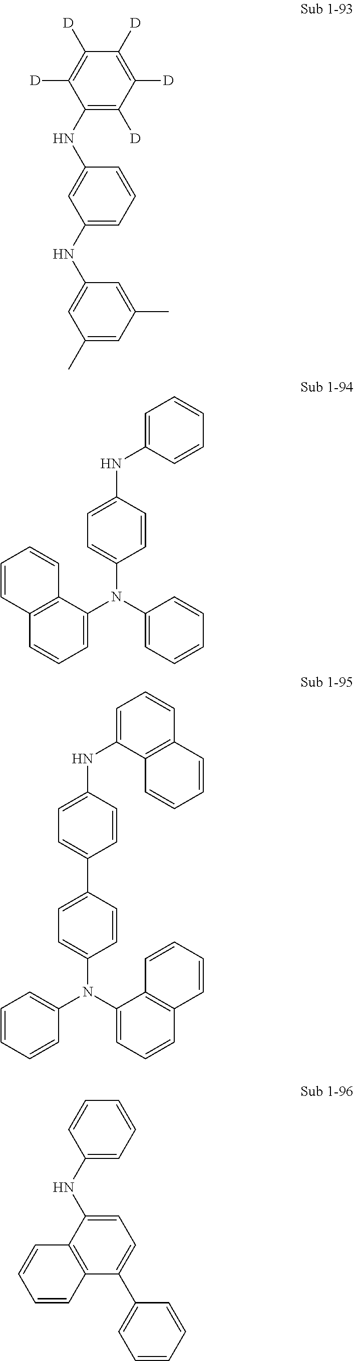

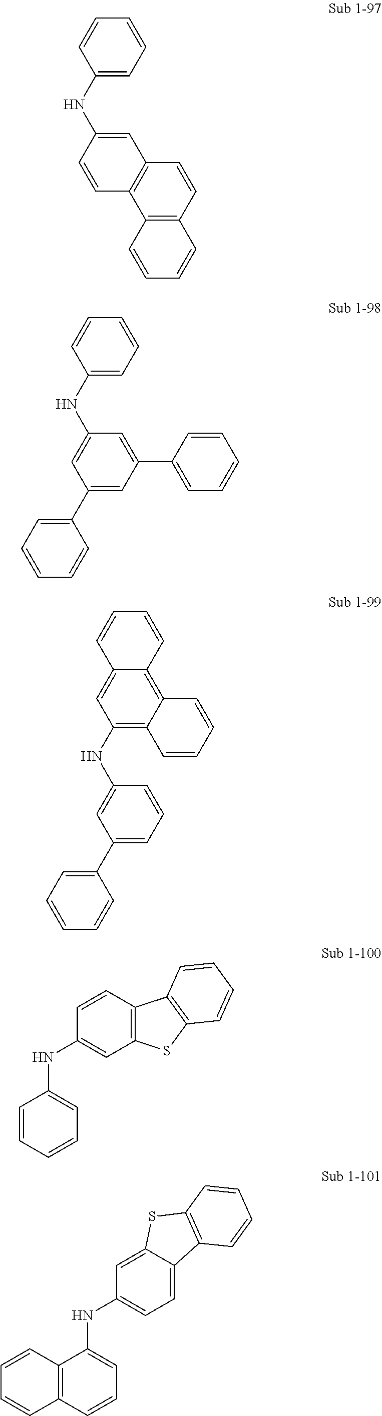

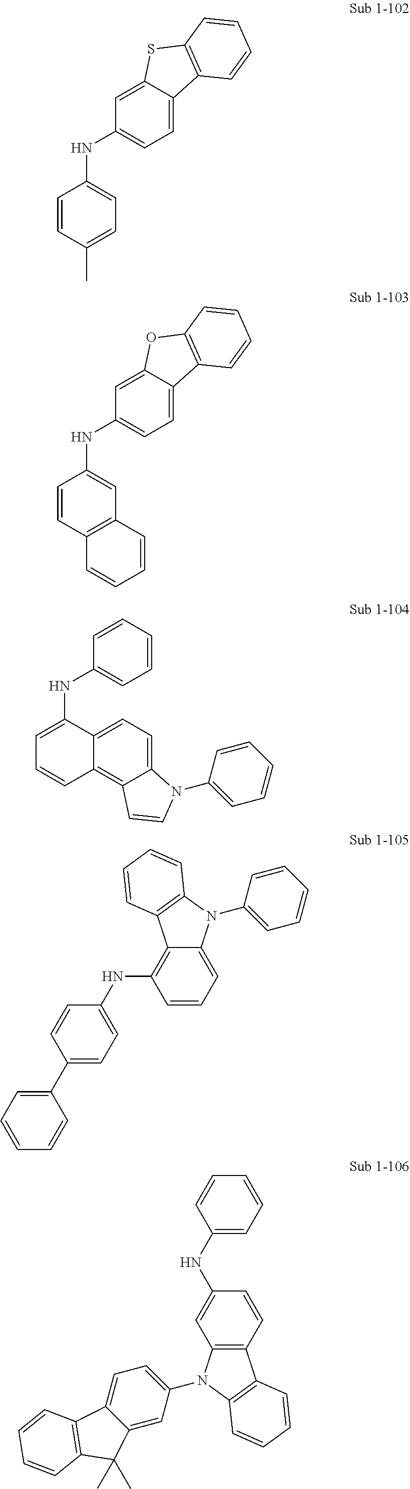

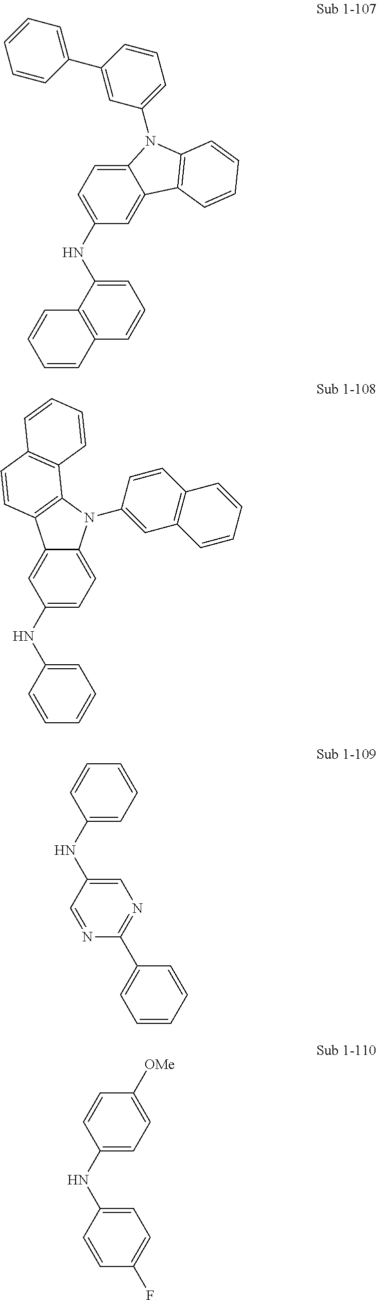

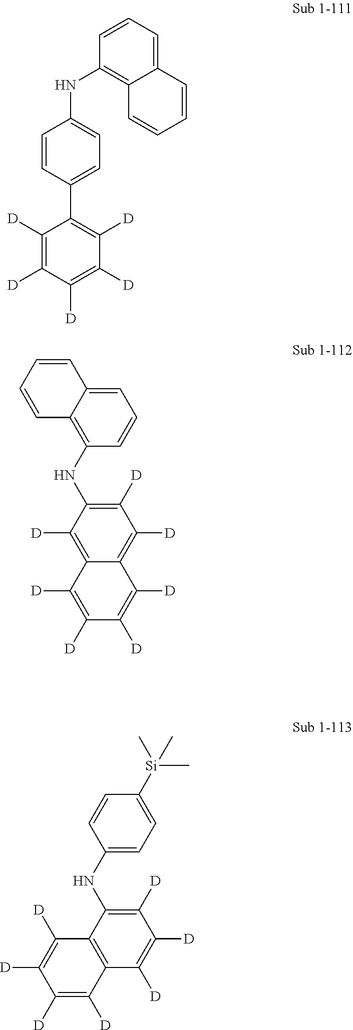

TABLE 1

| compound | FD-MS | compound | FD-MS |
|---|---|---|---|
| Sub 1-1 | m/z = 169.09(C₁₂H₁₁N = 169.22) | Sub 1-2 | m/z = 219.10(C₁₆H₁₃N = 219.28) |
| Sub 1-3 | m/z = 245.12(C₁₈H₁₅N = 245.32) | Sub 1-4 | m/z = 269.12(C₂₀H₁₅N = 269.34) |
| Sub 1-5 | m/z = 245.12(C₁₈H₁₅N = 245.32) | Sub 1-6 | m/z = 245.12(C₁₈H₁₅N = 245.32) |
| Sub 1-7 | m/z = 295.14(C₂₂H₁₇N = 295.38) | Sub 1-8 | m/z = 295.14(C₂₂H₁₇N = 295.38) |
| Sub 1-9 | m/z = 295.14(C₂₂H₁₇N = 295.38) | Sub 1-10 | m/z = 295.14(C₂₂H₁₇N = 295.38) |
| Sub 1-11 | m/z = 321.15(C₂₄H₁₉N = 321.41) | Sub 1-12 | m/z = 321.15(C₂₄H₁₉N = 321.41) |
| Sub 1-13 | m/z = 369.15(C₂₈H₁₉N = 369.47) | Sub 1-14 | m/z = 395.17(C₃₀H₂₁N = 395.51) |
| Sub 1-15 | m/z = 295.14(C₂₂H₁₇N = 295.38) | Sub 1-16 | m/z = 652.25(C₄₈H₃₂N₂O = 652.80) |
| Sub 1-17 | m/z = 371.17(C₂₈H₂₁N = 371.48) | Sub 1-18 | m/z = 371.17(C₂₈H₂₁N = 371.48) |
| Sub 1-19 | m/z = 421.18(C₃₂H₂₃N = 421.54) | Sub 1-20 | m/z = 371.17(C₂₈H₂₁N = 371.48) |
| Sub 1-21 | m/z = 447.20(C₃₄H₂₅N = 447.58) | Sub 1-22 | m/z = 336.16(C₂₄H₂₀N₂ = 336.43) |
| Sub 1-23 | m/z = 503.24(C₃₆H₂₉N₃ = 503.64) | Sub 1-24 | m/z = 285.15(C₂₁H₁₉N = 285.38) |
| Sub 1-25 | m/z = 335.17(C₂₅H₂₁N = 335.44) | Sub 1-26 | m/z = 335.17(C₂₅H₂₁N = 335.44) |
| Sub 1-27 | m/z = 361.18(C₂₇H₂₃N = 361.48) | Sub 1-28 | m/z = 451.23(C₃₄H₂₉N = 451.61) |
| Sub 1-29 | m/z = 401.21(C₃₀H₂₇N = 401.55) | Sub 1-30 | m/z = 477.25(C₃₆H₃₁N = 477.65) |
| Sub 1-31 | m/z = 391.14(C₂₇H₂₁NS = 391.53) | Sub 1-32 | m/z = 391.14(C₂₇H₂₁NS = 391.53) |
| Sub 1-33 | m/z = 375.16(C₂₇H₂₁NO = 375.46) | Sub 1-34 | m/z = 375.16(C₂₇H₂₁NO = 375.46) |
| Sub 1-35 | m/z = 459.20(C₃₅H₂₅N = 459.58) | Sub 1-36 | m/z = 423.20(C₃₂H₂₅N = 423.56) |
| Sub 1-37 | m/z = 586.24(C₄₄H₃₀N₂ = 586.74) | Sub 1-38 | m/z = 485.21(C₃₇H₂₇N = 485.63) |
| Sub 1-39 | m/z = 407.17(C₃₁H₂₁N = 407.52) | Sub 1-40 | m/z = 457.18(C₃₅H₂₃N = 457.58) |
| Sub 1-41 | m/z = 563.17(C₄₁H₂₅NS = 563.72) | Sub 1-42 | m/z = 626.27(C₄₇H₃₄N₂ = 626.80) |
| Sub 1-43 | m/z = 284.13(C₂₀H₁₆N₂ = 284.36) | Sub 1-44 | m/z = 246.12(C₁₇H₁₄N₂ = 246.31) |
| Sub 1-45 | m/z = 296.13(C₂₁H₁₆N₂ = 296.37) | Sub 1-46 | m/z = 334.15(C₂₄H₁₈N₂ = 334.42) |
| Sub 1-47 | m/z = 334.15(C₂₄H₁₈N₂ = 334.42) | Sub 1-48 | m/z = 460.19(C₃₄H₂₄N₂ = 460.58) |
| Sub 1-49 | m/z = 384.16(C₂₈H₂₀N₂ = 384.48) | Sub 1-50 | m/z = 500.19(C₃₆H₂₄N₂O = 500.60) |
| Sub 1-51 | m/z = 490.15(C₃₄H₂₂N₂S = 490.62) | Sub 1-52 | m/z = 225.06(C₁₄H₁₁NS = 225.31) |
| Sub 1-53 | m/z = 275.08(C₁₈H₁₃NS = 275.37) | Sub 1-54 | m/z = 275.08(C₁₈H₁₃NS = 275.37) |
| Sub 1-55 | m/z = 325.09(C₂₂H₁₅NS = 325.43) | Sub 1-56 | m/z = 351.11(C₂₄H₁₇NS = 351.47) |
| Sub 1-57 | m/z = 351.11(C₂₄H₁₇NS = 351.47) | Sub 1-58 | m/z = 351.11(C₂₄H₁₇NS = 351.47) |
| Sub 1-59 | m/z = 401.12(C₂₈H₁₉NS = 401.53) | Sub 1-60 | m/z = 401.12(C₂₈H₁₉NS = 401.53) |
| Sub 1-61 | m/z = 427.14(C₃₀H₂₁NS = 427.57) | Sub 1-62 | m/z = 381.06(C₂₄H₁₅NS₂ = 381.51) |
| Sub 1-63 | m/z = 381.06(C₂₄H₁₅NS₂ = 381.51) | Sub 1-64 | m/z = 452.13(C₃₁H₂₀N₂S = 452.58) |
| Sub 1-65 | m/z = 351.11(C₂₄H₁₇NS = 351.47) | Sub 1-66 | m/z = 325.09(C₂₂H₁₅NS = 325.43) |
| Sub 1-67 | m/z = 465.12(C₃₂H₁₉NOS = 465.57) | Sub 1-68 | m/z = 365.09(C₂₄H₁₅NOS = 365.45) |
| Sub 1-69 | m/z = 517.15(C₃₇H₂₃NOS = 517.65) | Sub 1-70 | m/z = 594.21(C₄₂H₃₀N₂S = 594.78) |
| Sub 1-71 | m/z = 259.10(C₁₈H₁₃NO = 259.31) | Sub 1-72 | m/z = 259.10(C₁₈H₁₃NO = 259.31) |
| Sub 1-73 | m/z = 259.10(C₁₈H₁₃NO = 259.31) | Sub 1-74 | m/z = 309.12(C₂₂H₁₅NO = 309.36) |
| Sub 1-75 | m/z = 335.13(C₂₄H₁₇NO = 335.40) | Sub 1-76 | m/z = 335.13(C₂₄H₁₇NO = 335.40) |
| Sub 1-77 | m/z = 335.13(C₂₄H₁₇NO = 335.40) | Sub 1-78 | m/z = 335.13(C₂₄H₁₇NO = 335.40) |
| Sub 1-79 | m/z = 485.18(C₃₆H₂₃NO = 485.59) | Sub 1-80 | m/z = 349.11(C₂₄H₁₅NO₂ = 349.39) |
| Sub 1-81 | m/z = 411.16(C₃₀H₂₁NO = 411.49) | Sub 1-82 | m/z = 225.15(C₁₆H₁₉N = 225.34) |
| Sub 1-83 | m/z = 275.17(C₂₀H₂₁N = 275.40) | Sub 1-84 | m/z = 234.12(C₁₆H₁₄N₂₋ = 234.30) |
| Sub 1-85 | m/z = 369.15(C₂₅H₂₀FNO = 369.44) | Sub 1-86 | m/z = 365.16(C₂₅H₂₃NSi = 365.55) |
| Sub 1-87 | m/z = 382.38(C₂₂H₁₄N₄O₃ = 382.38) | Sub 1-88 | m/z = 376.10(C₂₅H₁₆N₂S = 376.48) |
| Sub 1-89 | m/z = 322.15(C₂₃H₁₈N₂ = 322.41) | Sub 1-90 | m/z = 224.14(C₁₆H₈D₅N = 224.32) |
| Sub 1-91 | m/z = 250.15(C₁₈H₁₀D₅N = 250.36) | Sub 1-92 | m/z = 250.15(C₁₈H₁₀D₅N = 250.36) |
| Sub 1-93 | m/z = 278.18(C₂₀H₁₄D₅N = 278.41) | Sub 1-94 | m/z = 386.18(C₂₈H₂₂N₂ = 386.50) |
| Sub 1-95 | m/z = 512.23 (C₃₈H₂₈N₂ = 512.66) | Sub 1-96 | m/z = 295.14(C₂₂H₁₇N = 295.39) |
| Sub 1-97 | m/z = 269.12(C₂₀H₁₅N = 269.35) | Sub 1-98 | m/z = 321.15(C₂₄H₁₉N = 321.42) |
| Sub 1-99 | m/z = 346.15(C₂₅H₁₈N₂ = 346.43) | Sub 1-100 | m/z = 275.08(C₁₈H₁₃NS = 275.37) |
| Sub 1-101 | m/z = 325.09(C₂₂H₁₅NS = 325.43) | Sub 1-102 | m/z = 290.09(C₁₈H₁₄N₂S = 290.38) |
| Sub 1-103 | m/z = 309.12(C₂₂H₁₅NO = 309.37) | Sub 1-104 | m/z = 334.15(C₂₄H₁₈N₂ = 334.42) |
| Sub 1-105 | m/z = 410.18(C₃₀H₂₂N₂ = 410.52) | Sub 1-106 | m/z = 450.21(C₃₃H₂₆N₂ = 450.59) |
| Sub 1-107 | m/z = 460.19(C₃₄H₂₄N₂ = 460.58) | Sub 1-108 | m/z = 434.18(C₃₂H₂₂N₂ = 434.54) |
| Sub 1-109 | m/z = 247.11(C₁₆H₁₃N₃ = 247.30) | Sub 1-110 | m/z = 217.09(C₁₃H₁₂FNO = 217.24) |
| Sub 1-111 | m/z = 300.17(C₂₂H₁₂D₅N = 300.42) | Sub 1-112 | m/z = 276.16(C₂₀H₈D₇N = 276.39) |
| Sub 1-113 | m/z = 298.19(C₁₉H₁₄D₇NSi = 298.51) | | |

### 2. Synthesis of Sub 2

Sub 2 of Reaction Scheme 1 can be synthesized by the reaction path of the following Reaction Scheme 4 or 5, but is not limited thereto.

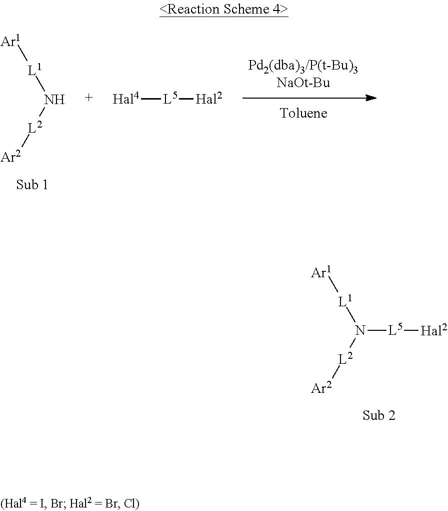

L¹ and L² are single bonds, and Ar¹ and Are form a ring. (Hal³=I, Br)

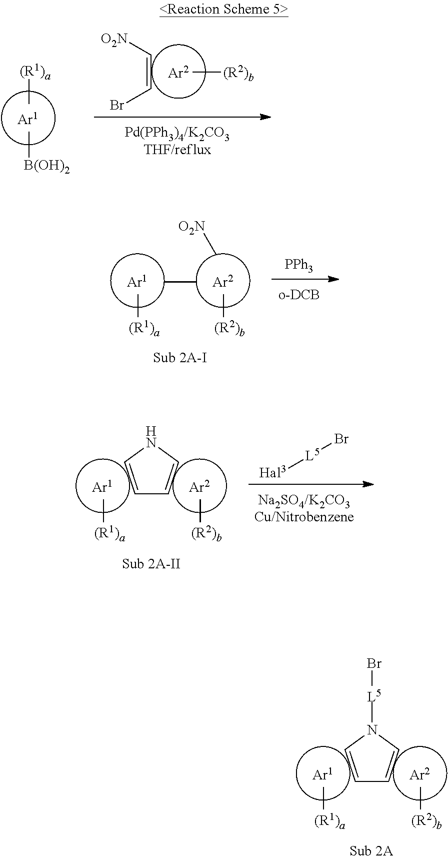

Examples of synthesis of specific compounds belonging to Sub 2 and Sub 2A are as follows.

### Synthesis of Sub 2A-1

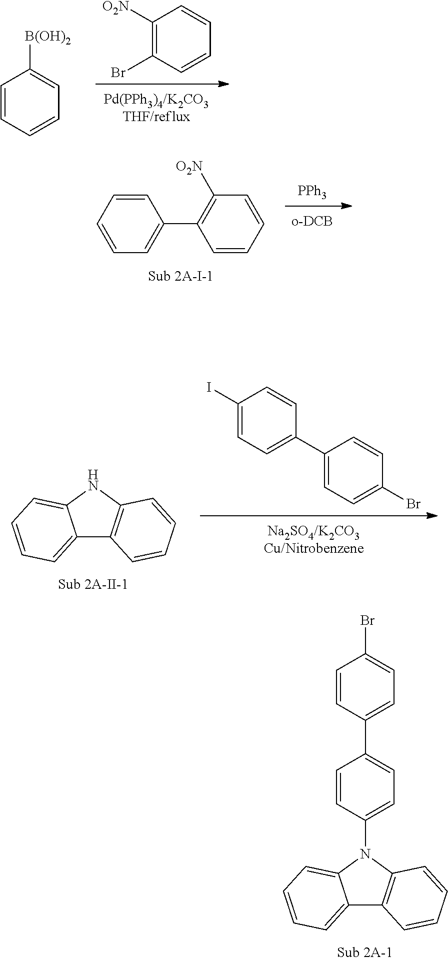

### 1) Synthesis of Intermediate Sub 2A-I-1

phenyl boronic acid (66.4 g, 544.5 mmol) was dissolved in THF (2396 mL) in a round bottom flask, and 1-bromo-2-nitrobenzene (110 g, 544.5 mmol), Pd(PPh₃)₄ (18.9 g, 16.3 mmol), K₂CO₃ (225.8 g, 1633.6 mmol), and water (1198 ml) were added and refluxed with stirring. After the reaction was completed, the reaction mixture was extracted with ether and water. The organic layer was dried over MgSO₄ and concentrated. The resulting organic material was separated by silicagel column chromatography and recrystallization to obtain 99.8 g (yield: 92%) of the product.

### 2) Synthesis of Intermediate Sub 2A-II-1

Sub 2A-I-1 (95 g, 476.9 mmol), Triphenylphosphine (375.2 g, 1430.7 mmol), o-Dichlorobenzene (1907.5 ml) were added in a round bottom flask and refluxed at 180° C. After the reaction was completed, the reaction mixture was cooled to room temperature and extracted with methylenechloride and water. The organic layer was dried over MgSO₄ and concentrated. The resulting organic material was separated by silicagel column chromatography and recrystallization to obtain 67 g (yield: 84%) of the product.

### 3) Synthesis of Sub 2A-1

Sub 2A-II-1 (59 g, 352.9 mmol) was dissolved in nitrobenzene (1765 ml) in a round bottom flask, and 4-bromo-4′-iodo-1,1′-biphenyl (139.3 g, 388.1 mmol), Na₂SO₄ (50.1 g, 352.9 mmol), K₂CO₃ (48.8 g, 352.9 mmol), Cu (6.7 g, 105.9 mmol) were added and stirred at 200° C. When the reaction was complete, nitrobenzene was removed by distillation and extracted with CH₂Cl₂ and water. The organic layer was dried over MgSO₄ and concentrated. The resulting compound was separated by silicagel column chromatography and recrystallization to obtain 102.6 g (yield: 73%) of the product.

### Synthesis of Sub 2A-9

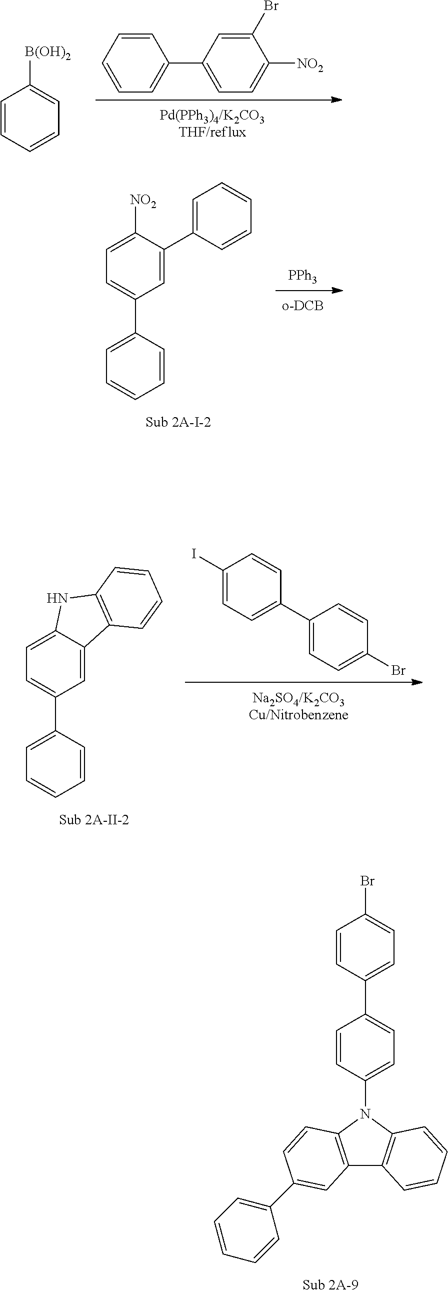

### 1) Synthesis of Intermediate Sub 2A-I-2

phenylboronic acid (65.8 g, 539.4 mmol), THF (2373 ml), 3-bromo-4-nitro-1,1′-biphenyl (150 g, 539.4 mmol), Pd(PPh₃)₄ (18.7 g, 16.2 mmol), K₂CO₃ (223.6 g, 1618 mmol) and water (1187 ml) were carried out in the same manner as Sub 2A-I-1 to obtain 106.9 g of the product. (Yield: 72%)

### 2) Synthesis of Intermediate Sub 2A-II-2

Sub 2A-I-2 (100 g, 363.2 mmol), Triphenylphosphine (285.8 g, 1089.7 mmol), o-Dichlorobenzene (1453 mL) were carried out in the same manner as Sub 2A-II-1 to obtain 54.8 g of the product. (Yield: 62%)

### 3) Synthesis of Sub 2A-9

Sub 2A-II-2 (40 g, 164.4 mmol), nitrobenzene (822 ml), 4-bromo-4′-iodo-1,1′-biphenyl (64.9 g, 180.8 mmol), Na₂SO₄ (23.4 g, 164.4 mmol), K₂CO₃ (22.7 g, 164.4 mmol), Cu (3.1 g, 49.3 mmol) were carried out in the same manner as Sub 2A-1 to obtain 55.4 g of the product. (Yield: 71%)

### Synthesis of Sub 2A-18

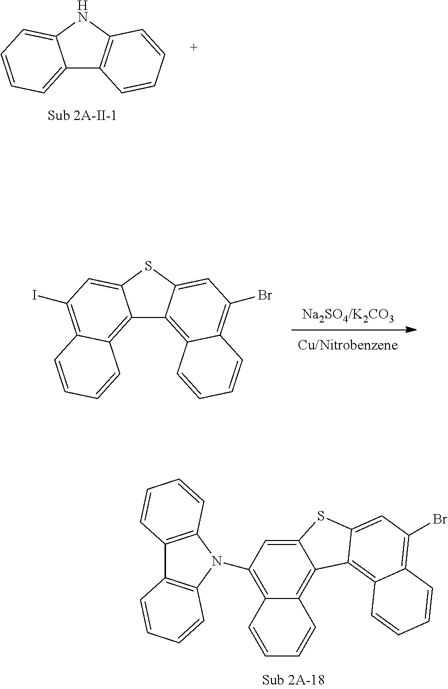 [SMILES: c1ccc2c(c1)[nH]c1ccccc12 ; Brc1cc2sc3cc(I)c4ccccc4c3c2c2ccccc12 ; Brc1cc2sc3cc(-n4c5ccccc5c5ccccc54)c4ccccc4c3c2c2ccccc12]

Sub 2A-II-1 (30 g, 179.4 mmol), nitrobenzene (897 ml), 5-bromo-9-iododinaphtho[2,1-b:1′,2′-d]thiophene (96.5 g, 197.4 mmol), Na₂SO₄ (25.5 g, 179.4 mol), K₂CO₃ (24.8 g, 179.4 mmol), Cu (3.4 g, 53.8 mmol) were carried out in the same manner as Sub 2A-1 to obtain 61.6 g of the product. (Yield: 65%)

### Synthesis of Sub 2A-20

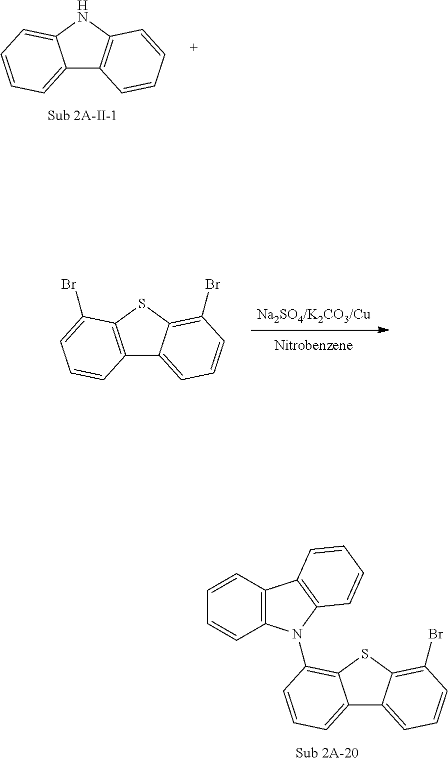 [SMILES: c1ccc2c(c1)[nH]c1ccccc12 ; Brc1cccc2c1sc1c(Br)cccc12 ; Brc1cccc2c1sc1c(-n3c4ccccc4c4ccccc43)cccc12]

Sub 2A-II-1 (30 g, 179.4 mmol), nitrobenzene (897 ml), 2-bromo-7-iodo-9,9-diphenyl-9H-fluorene (78.8 g, 197.4 mmol), Na₂SO₄ (25.5 g, 179.4 mmol), K₂CO₃ (24.8 g, 179.4 mmol), Cu (3.4 g, 53.8 mmol) were carried out in the same manner as Sub 2A-1 to obtain 53 g of the product. (Yield: 69%)

### Synthesis of Sub 2A-19

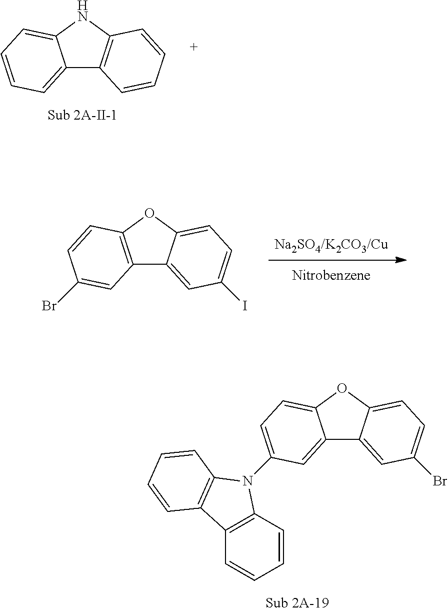 [SMILES: c1ccc2c(c1)[nH]c1ccccc12 ; Brc1ccc2oc3ccc(I)cc3c2c1 ; Brc1ccc2oc3ccc(-n4c5ccccc5c5ccccc54)cc3c2c1]

Sub 2A-II-1 (30 g, 179.4 mmol), nitrobenzene (897 ml), 2-bromo-7-iodo-9,9-diphenyl-9H-fluorene (78.8 g, 197.4 mmol), Na₂SO₄ (25.5 g, 179.4 mmol), K₂CO₃ (24.8 g, 179.4 mmol), Cu (3.4 g, 53.8 mmol) were carried out in the same manner as Sub 2A-1 to obtain 49.6 g of the product. (Yield: 67%)

### Synthesis of Sub 2A-22

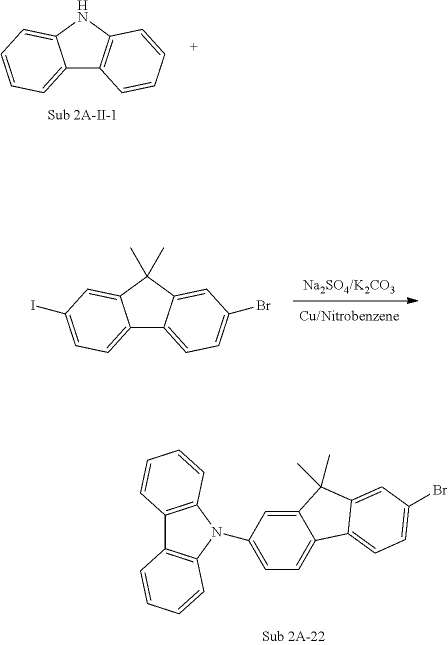 [SMILES: c1ccc2c(c1)[nH]c1ccccc12 ; CC1(C)c2cc(Br)ccc2-c2ccc(I)cc21 ; CC1(C)c2cc(Br)ccc2-c2ccc(-n3c4ccccc4c4ccccc43)cc21]

Sub 2A-II-1 (30 g, 179.4 mmol), nitrobenzene (897 ml), 2-bromo-7-iodo-9,9-dimethyl-9H-fluorene (78.8 g, 197.4 mmol), Na₂SO₄ (25.5 g, 179.4 mmol), K₂CO₃ (24.8 g, 179.4 mmol), Cu (3.4 g, 53.8 mmol) were carried out in the same manner as Sub 2A-1 to obtain 52.7 g of the product. (Yield: 67%)

### Synthesis of Sub 2A-33

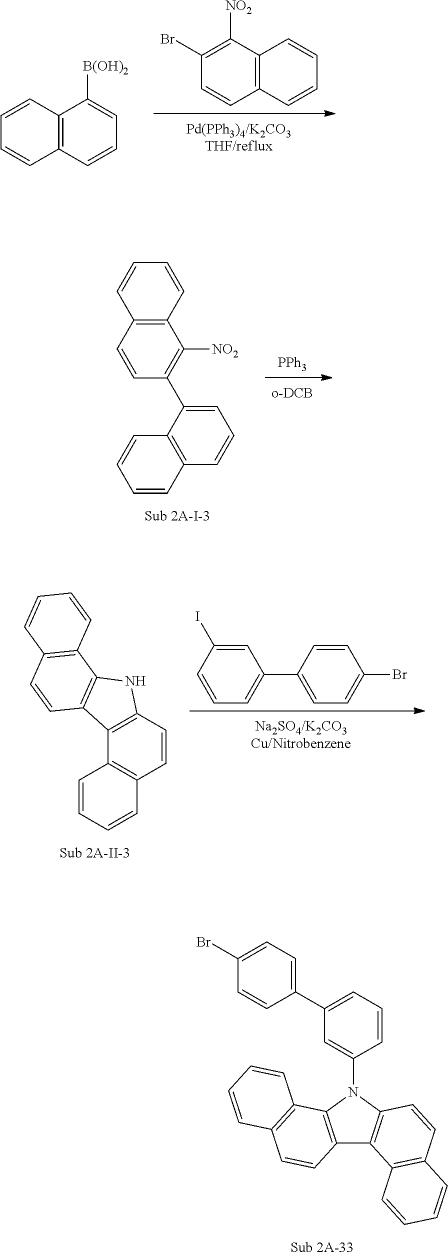

### 1) Synthesis of Intermediate Sub 2A-I-3

naphthalen-1-ylboronic acid (68.2 g, 396.7 mmol), THF (1745 ml), 2-bromo-1-nitronaphthalene (100 g, 396.7 mmol), Pd(PPh₃)₄ (13.8 g, 11.9 mmol), K₂CO₃ (164.5 g, 1190 mmol) and water (873 ml) were carried out in the same manner as Sub 2A-I-1 to obtain 83.1 g of the product. (Yield: 70%)

### 2) Synthesis of Intermediate Sub 2A-II-3

Sub 2A-I-3 (80 g, 267.3 mmol), Triphenylphosphine (210.3 g, 801.8 mmol), o-Dichlorobenzene (1069 mL) were carried out in the same manner as Sub 2A-II-1 to obtain 45.7 g of the product. (Yield: 64%)

### 3) Synthesis of Sub 2A-33

Sub 2A-II-3 (45 g, 168.3 mmol), nitrobenzene (842 ml), 4′-bromo-3-iodo-1,1′-biphenyl (66.5 g, 185.2 m mol), Na₂SO₄ (23.9 g, 168.3 mmol), K₂CO₃ (23.3 g, 168.3 mmol), Cu (3.2 g, 50.5 mmol) were carried out in the same manner as Sub 2A-1 to obtain 50.3 g of the product. (Yield: 60%)

### Synthesis of Sub 2A-34

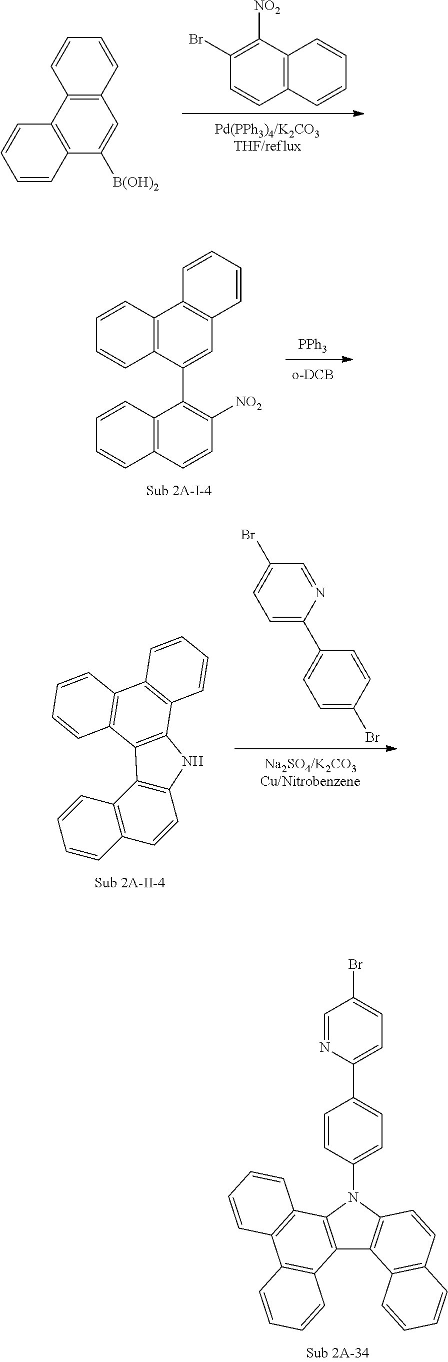

### 1) Synthesis of Intermediate Sub 2A-I-4

naphthalen-1-ylboronic acid (44.05 g, 198.36 mmol), THF (873 ml), 2-bromo-1-nitronaphthalene (50 g, 198.36 mmol), Pd(PPh₃)₄ (6.88 g, 5.95 mmol), K₂CO₃ (82.25 g, 595.07 mmol) and water (436 ml) were carried out in the same manner as Sub 2A-I-1 to obtain 57.52 g of the product. (Yield: 83%)

### 2) Synthesis of Intermediate Sub 2A-II-4

Sub 2A-I-4 (57.52 g, 164.63 mmol), Triphenylphosphine (107.95 g, 411.57 mmol), o-Dichlorobenzene (823 ml) were carried out in the same manner as Sub 2A-II-1 to obtain 22.99 g of the product. (Yield: 44%)

### 3) Synthesis of Sub 2A-34

Sub 2A-II-4 (22.99 g, 72.44 mmol), nitrobenzene (362 ml), 4′-bromo-3-iodo-1,1′-biphenyl (22.67 g, 72.44 mmol), Na₂SO₄ (5.14 g, 36.22 mmol), K₂CO₃ (5.01 g, 36.22 mmol), Cu (0.69 g, 10.87 mmol) were carried out in the same manner as Sub 2A-1 to obtain 26.27 g of the product. (Yield: 66%)

### Synthesis of Sub 2-1

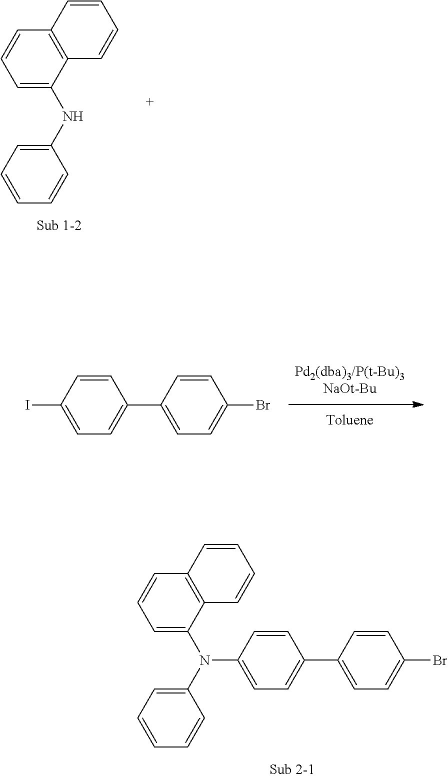 [SMILES: c1ccc(Nc2cccc3ccccc23)cc1 ; Brc1ccc(-c2ccc(I)cc2)cc1 ; Brc1ccc(-c2ccc(N(c3ccccc3)c3cccc4ccccc34)cc2)cc1]

Sub 1-2 (20.16 g, 91.92 mmol) was dissolved in toluene (965 ml) in a round bottom flask, and 4-bromo-4′-iodo-1,1′-biphenyl (33 g, 91.92 mmol), Pd₂(dba)₃ (1.26 g, 1.38 mmol), P(t-Bu)₃ (0.56 g, 2.76 mmol), NaOt-Bu (13.25 g, 137.88 mmol) were added and stirred at 70° C. When the reaction was complete, the reaction mixture was extracted with CH₂Cl₂ and water. The organic layer was dried over MgSO₄ and concentrated. The resulting compound was separated by silicagel column chromatography and recrystallization to obtain 28.15 g (yield: 68%) of the product.

### Synthesis of Sub 2-7

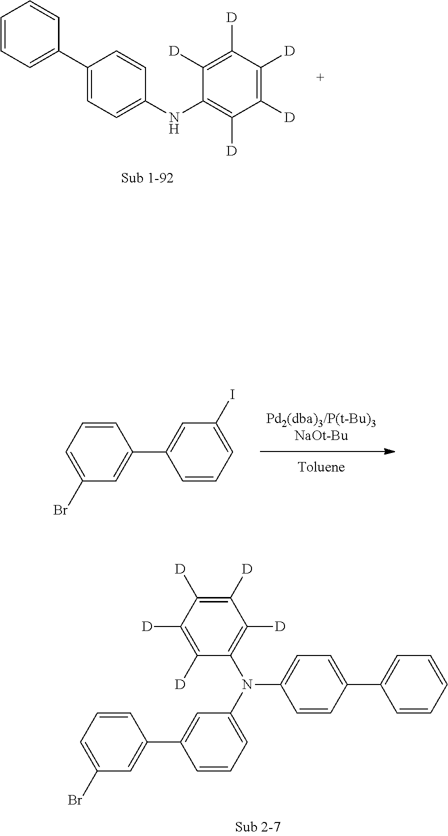 [SMILES: [2H]c1c([2H])c([2H])c(Nc2ccc(-c3ccccc3)cc2)c([2H])c1[2H] ; Brc1cccc(-c2cccc(I)c2)c1 ; [2H]c1c([2H])c([2H])c(N(c2ccc(-c3ccccc3)cc2)c2cccc(-c3cccc(Br)c3)c2)c([2H])c1[2H]]

Sub 1-92 (17.43 g, 69.64 mmol), toluene (731 ml), 3-bromo-3′-iodo-1,1′-biphenyl (25 g, 69.64 mmol), Pd₂(dba)₃ (0.96 g, 1.04 mmol), P(t-Bu)₃ (0.42 g, 2.09 mmol), NaOt-Bu (10.04 g, 104.46 mmol) were carried out in the same manner as Sub 2-1 to obtain 23.13 g of the product. (Yield: 69%)

### Synthesis of Sub 2-14

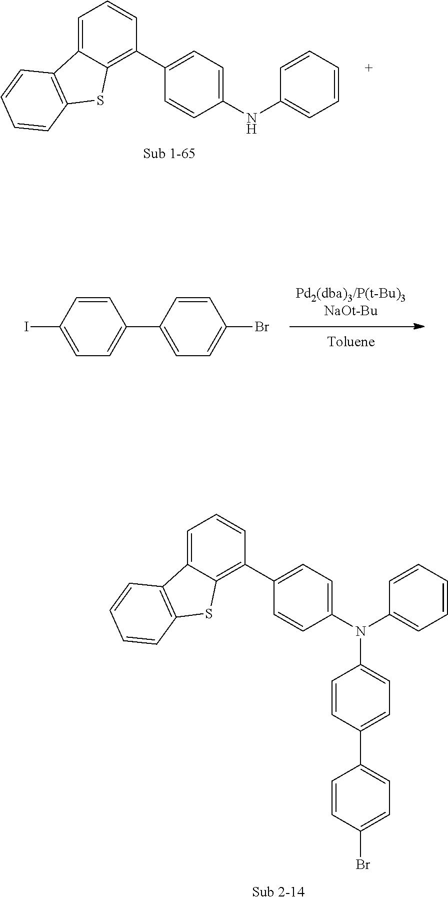 [SMILES: c1ccc(Nc2ccc(-c3cccc4c3sc3ccccc34)cc2)cc1 ; Brc1ccc(-c2ccc(I)cc2)cc1 ; Brc1ccc(-c2ccc(N(c3ccccc3)c3ccc(-c4cccc5c4sc4ccccc45)cc3)cc2)cc1]

Sub 1-65 (24.48 g, 69.64 mmol), toluene (731 ml), 2-bromo-6-iodonaphthalene (25 g, 69.64 mmol), Pd₂(dba)₃ (0.96 g, 1.04 mmol), P(t-Bu)₃ (0.42 g, 2.09 mmol), NaOt-Bu (10.04 g, 104.46 mmol) were carried out in the same manner as Sub 2-1 to obtain 27.18 g of the product. (Yield: 67%)

### Synthesis of Sub 2-28

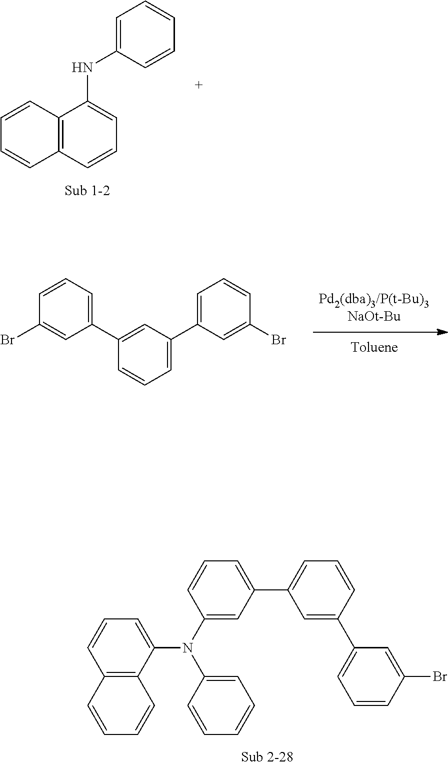 [SMILES: c1ccc(Nc2cccc3ccccc23)cc1 ; Brc1cccc(-c2cccc(-c3cccc(Br)c3)c2)c1 ; Brc1cccc(-c2cccc(-c3cccc(N(c4ccccc4)c4cccc5ccccc45)c3)c2)c1]

Sub 1-2 (21.87 g, 66.99 mmol), toluene (703 ml), 3,3″-dibromo-1,1′:3′,1″-terphenyl (26 g, 66.99 mmol), Pd₂(dba)₃ (0.92 g, 1 mmol), P(t-Bu)₃ (0.41 g, 2.01 mmol), NaOt-Bu (9.66 g, 100.49 mmol) were carried out in the same manner as Sub 2-1 to obtain 21.87 g of the product. (Yield: 62%)

### Synthesis of Sub 2-29

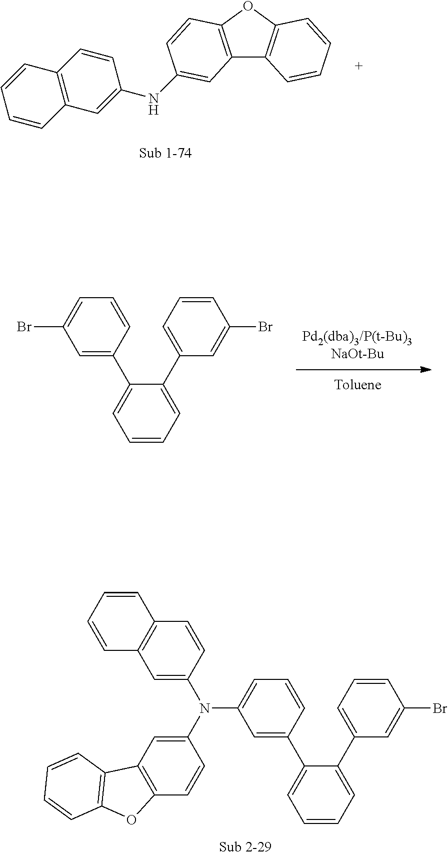 [SMILES: c1ccc2cc(Nc3ccc4oc5ccccc5c4c3)ccc2c1 ; Brc1cccc(-c2ccccc2-c2cccc(Br)c2)c1 ; Brc1cccc(-c2ccccc2-c2cccc(N(c3ccc4ccccc4c3)c3ccc4oc5ccccc5c4c3)c2)c1]

Sub 1-74 (20.73 g, 66.99 mmol), toluene (703 ml), 3,3″-dibromo-1,1′:2′,1″-terphenyl (26 g, 66.99 mmol), Pd₂(dba)₃ (0.92 g, 1 mmol), P(t-Bu)₃ (0.41 g, 2.01 mmol), NaOt-Bu (9.66 g, 100.49 mmol) were carried out in the same manner as Sub 2-1 to obtain 24.78 g of the product. (Yield: 60%)

### Synthesis of Sub 2-36

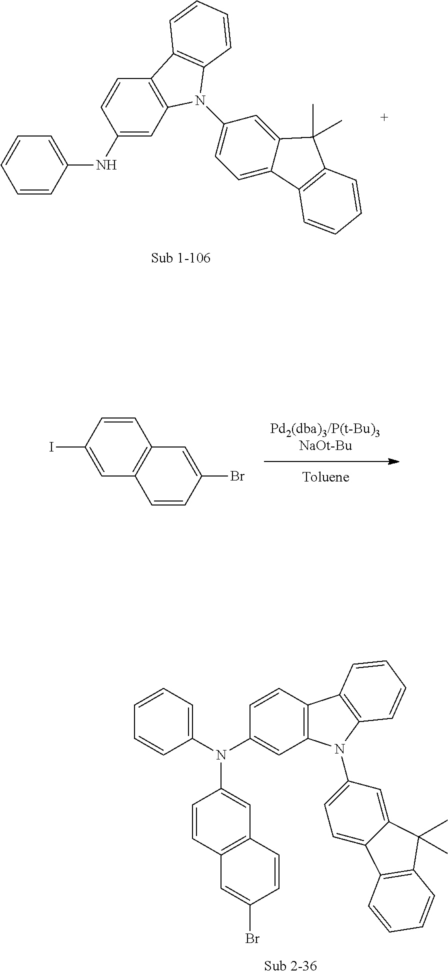 [SMILES: CC1(C)c2ccccc2-c2ccc(-n3c4ccccc4c4ccc(Nc5ccccc5)cc43)cc21 ; Brc1ccc2cc(I)ccc2c1 ; CC1(C)c2ccccc2-c2ccc(-n3c4ccccc4c4ccc(N(c5ccccc5)c5ccc6cc(Br)ccc6c5)cc43)cc21]

Sub 1-106 (31.12 g, 69.08 mmol), toluene (725 ml), 2-bromo-6-iodonaphthalene (23 g, 69.08 mmol), Pd₂(dba)₃ (0.95 g, 1.04 mmol), P(t-Bu)₃ (0.42 g, 2.07 mmol), NaOt-Bu (9.96 g, 103.61 mmol) were carried out in the same manner as Sub 2-1 to obtain 28.98 g of the product. (Yield: 64%)

### Synthesis of Sub 2-44

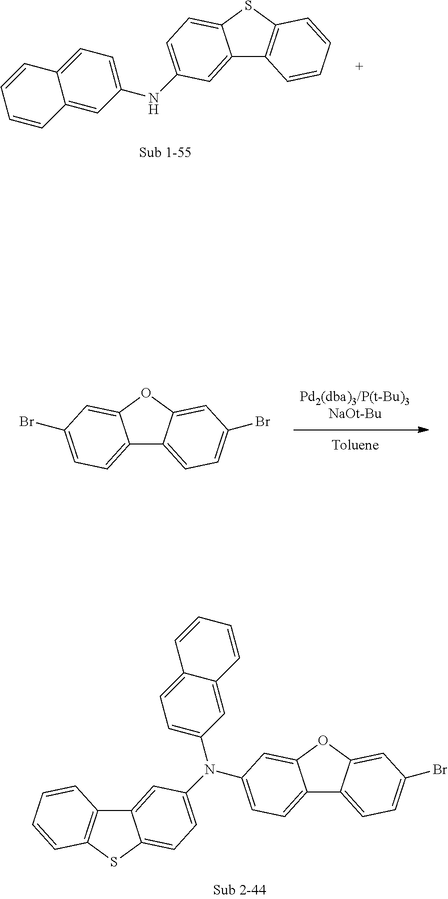 [SMILES: c1ccc2cc(Nc3ccc4sc5ccccc5c4c3)ccc2c1 ; Brc1ccc2c(c1)oc1cc(Br)ccc12 ; Brc1ccc2c(c1)oc1cc(N(c3ccc4ccccc4c3)c3ccc4sc5ccccc5c4c3)ccc12]

Sub 1-55 (25.96 g, 79.76 mmol), toluene (837 ml), 3,7-dibromodibenzo[b,d]furan (26 g, 79.76 mmol), Pd₂(dba)₃ (1.10 g, 1.20 mmol), P(t-Bu)₃ (0.48 g, 2.39 mmol), NaOt-Bu (11.5 g, 119.64 mmol) were carried out in the same manner as Sub 2-1 to obtain 30.94 g of the product. (Yield: 68%)

### Synthesis of Sub 2-56

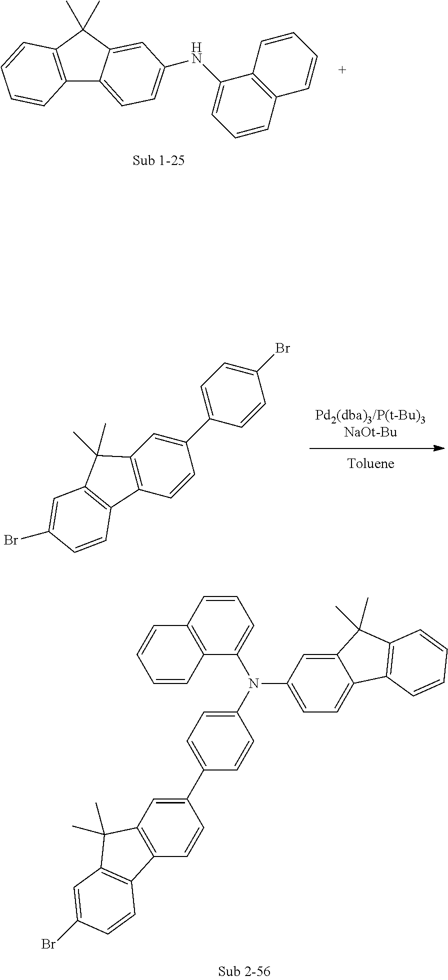 [SMILES: CC1(C)c2ccccc2-c2ccc(Nc3cccc4ccccc34)cc21 ; CC1(C)c2cc(Br)ccc2-c2ccc(-c3ccc(Br)cc3)cc21 ; CC1(C)c2cc(Br)ccc2-c2ccc(-c3ccc(N(c4ccc5c(c4)C(C)(C)c4ccccc4-5)c4cccc5ccccc45)cc3)cc21]

Sub 1-25 (20.37 g, 60.72 mmol), toluene (638 ml), 2-bromo-7-(4-bromophenyl)-9,9-dimethyl-9H-fluorene (26 g, 60.72 mmol), Pd₂(dba)₃ (0.83 g, 0.91 mmol), P(t-Bu)₃ (0.37 g, 1.82 mmol), NaOt-Bu (8.75 g, 91.09 mmol) were carried out in the same manner as Sub 2-1 to obtain 25.70 g of the product. (Yield: 62%)

### Synthesis of Sub 2-59

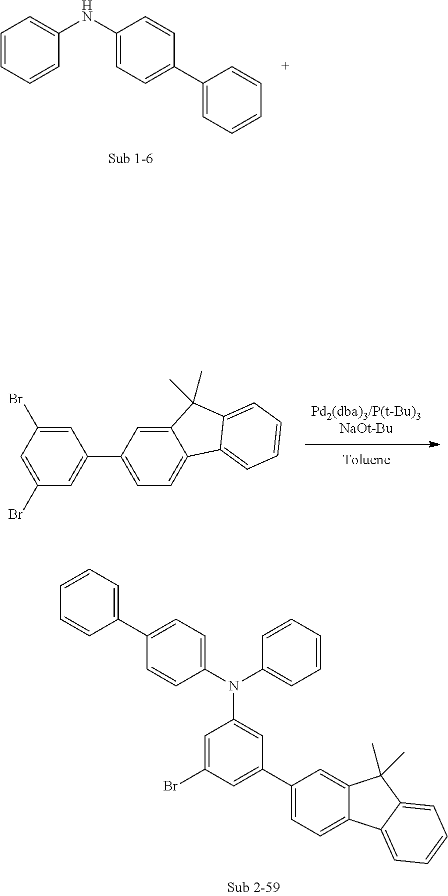 [SMILES: c1ccc(Nc2ccc(-c3ccccc3)cc2)cc1 ; CC1(C)c2ccccc2-c2ccc(-c3cc(Br)cc(Br)c3)cc21 ; CC1(C)c2ccccc2-c2ccc(-c3cc(Br)cc(N(c4ccccc4)c4ccc(-c5ccccc5)cc4)c3)cc21]

Sub 1-6 (14.90 g, 60.72 mmol), toluene (638 ml), 2-(3,5-dibromophenyl)-9,9-dimethyl-9H-fluorene (26 g, 60.72 mmol), Pd₂(dba)₃ (0.83 g, 0.91 mmol), P(t-Bu)₃ (0.37 g, 1.82 mmol), NaOt-Bu (8.75 g, 91.09 mmol) were carried out in the same manner as Sub 2-1 to obtain 21.23 g of the product. (Yield: 59%)

Examples of Sub 2 and Sub 2A are as follows, but are not limited thereto.

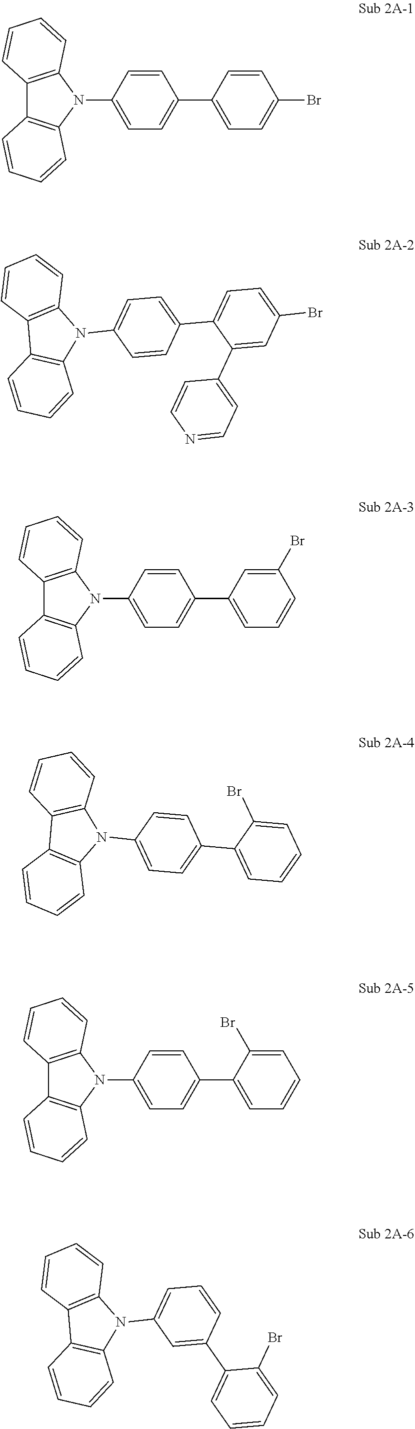 [SMILES: Brc1ccc(-c2ccc(-n3c4ccccc4c4ccccc43)cc2)cc1 ; Brc1ccc(-c2ccc(-n3c4ccccc4c4ccccc43)cc2)c(-c2ccncc2)c1 ; Brc1cccc(-c2ccc(-n3c4ccccc4c4ccccc43)cc2)c1 ; Brc1ccccc1-c1ccc(-n2c3ccccc3c3ccccc32)cc1 ; Brc1ccccc1-c1ccc(-n2c3ccccc3c3ccccc32)cc1 ; Brc1ccccc1-c1cccc(-n2c3ccccc3c3ccccc32)c1]

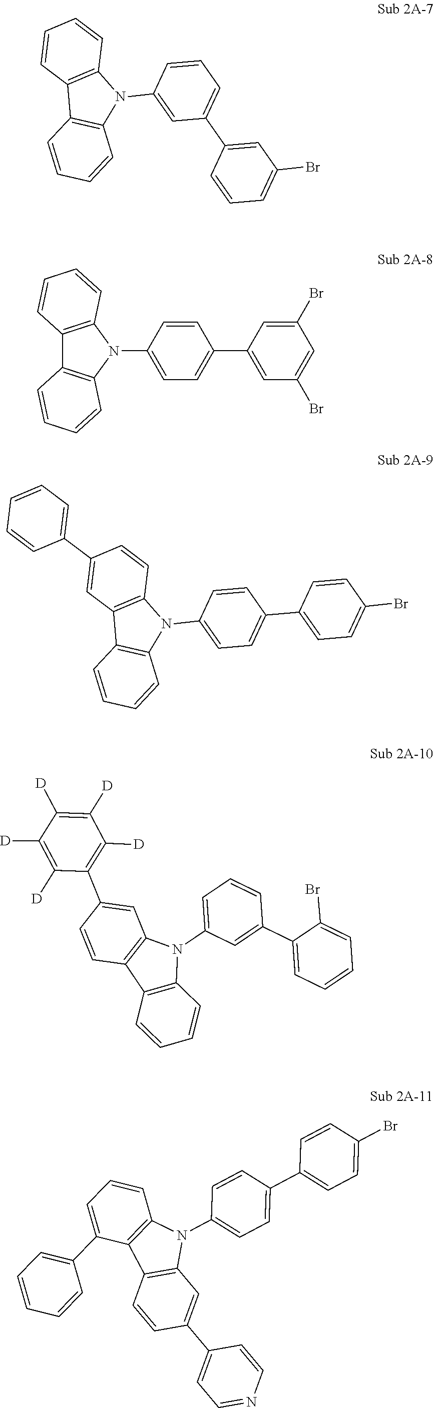 [SMILES: Brc1cccc(-c2cccc(-n3c4ccccc4c4ccccc43)c2)c1 ; Brc1cc(Br)cc(-c2ccc(-n3c4ccccc4c4ccccc43)cc2)c1 ; Brc1ccc(-c2ccc(-n3c4ccccc4c4cc(-c5ccccc5)ccc43)cc2)cc1 ; [2H]c1c([2H])c([2H])c(-c2ccc3c4ccccc4n(-c4cccc(-c5ccccc5Br)c4)c3c2)c([2H])c1[2H] ; Brc1ccc(-c2ccc(-n3c4cc(-c5ccncc5)ccc4c4c(-c5ccccc5)cccc43)cc2)cc1]

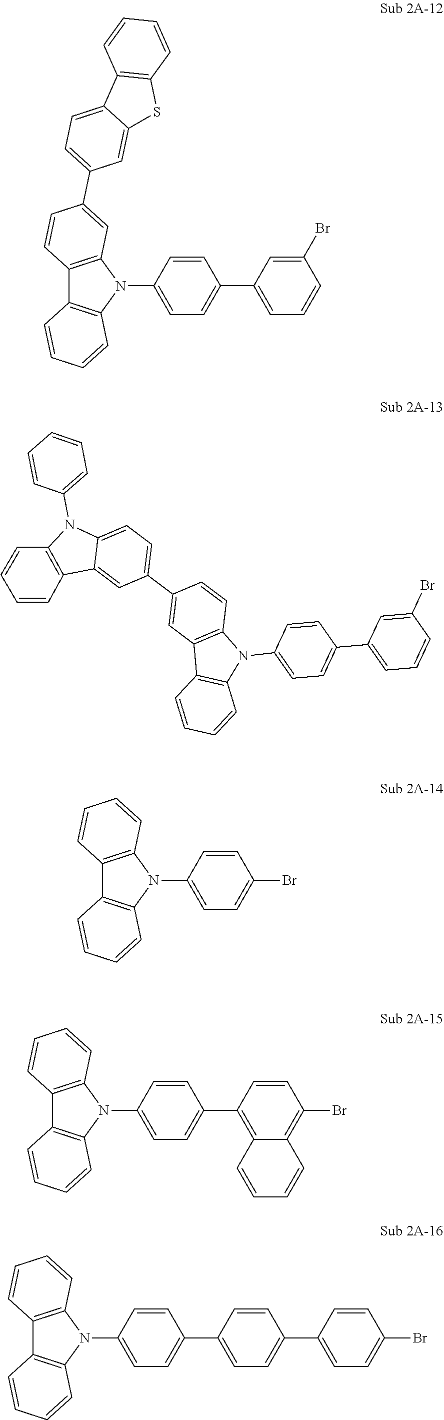 [SMILES: Brc1cccc(-c2ccc(-n3c4ccccc4c4ccc(-c5ccc6c(c5)sc5ccccc56)cc43)cc2)c1 ; Brc1cccc(-c2ccc(-n3c4ccccc4c4cc(-c5ccc6c(c5)c5ccccc5n6-c5ccccc5)ccc43)cc2)c1 ; Brc1ccc(-n2c3ccccc3c3ccccc32)cc1 ; Brc1ccc(-c2ccc(-n3c4ccccc4c4ccccc43)cc2)c2ccccc12 ; Brc1ccc(-c2ccc(-c3ccc(-n4c5ccccc5c5ccccc54)cc3)cc2)cc1]

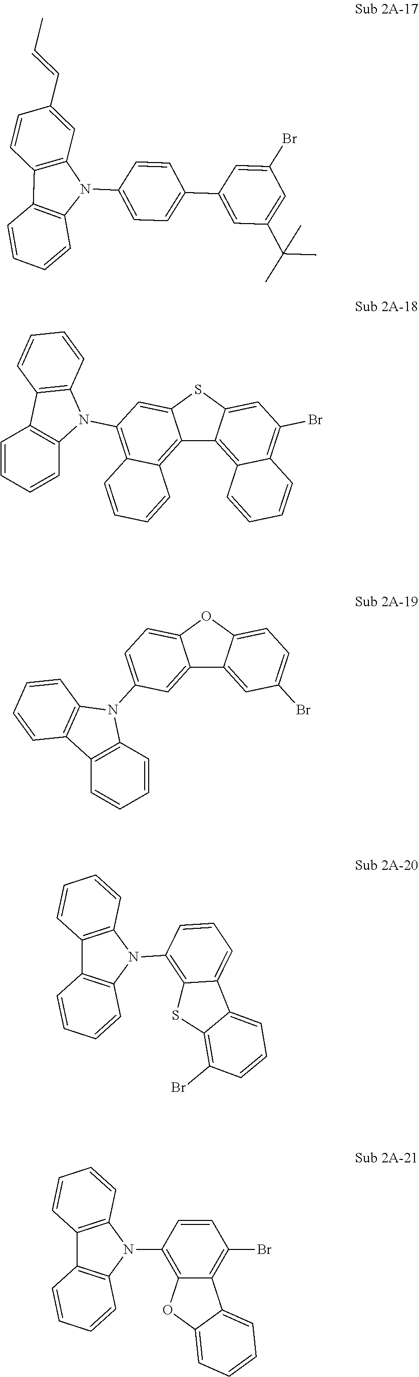 [SMILES: C/C=C/c1ccc2c3ccccc3n(-c3ccc(-c4cc(Br)cc(C(C)(C)C)c4)cc3)c2c1 ; Brc1cc2sc3cc(-n4c5ccccc5c5ccccc54)c4ccccc4c3c2c2ccccc12 ; Brc1ccc2oc3ccc(-n4c5ccccc5c5ccccc54)cc3c2c1 ; Brc1cccc2c1sc1c(-n3c4ccccc4c4ccccc43)cccc12 ; Brc1ccc(-n2c3ccccc3c3ccccc32)c2oc3ccccc3c12]

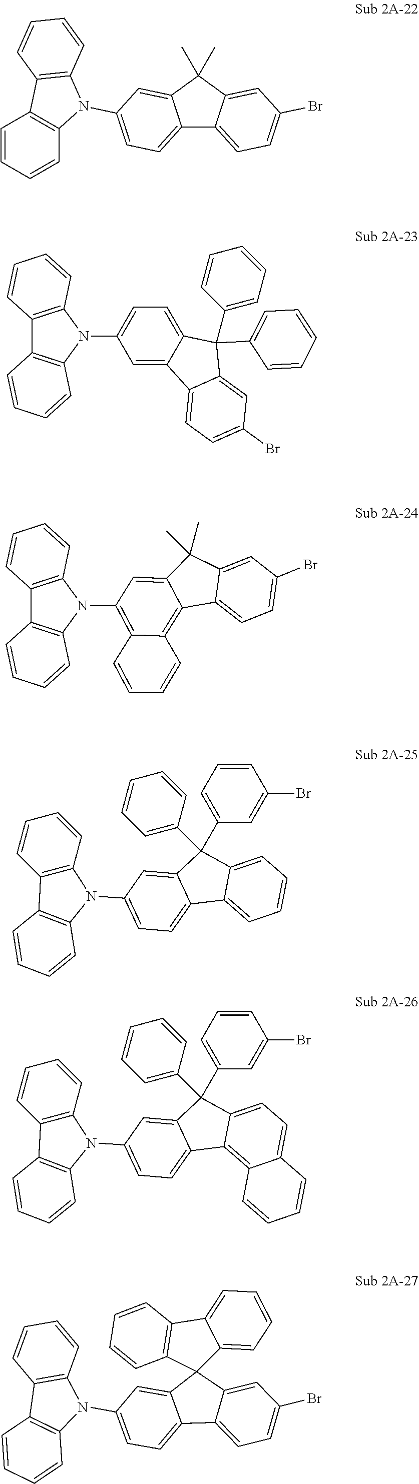 [SMILES: CC1(C)c2cc(Br)ccc2-c2ccc(-n3c4ccccc4c4ccccc43)cc21 ; Brc1ccc2c(c1)C(c1ccccc1)(c1ccccc1)c1ccc(-n3c4ccccc4c4ccccc43)cc1-2 ; CC1(C)c2cc(Br)ccc2-c2c1cc(-n1c3ccccc3c3ccccc31)c1ccccc21 ; Brc1cccc(C2(c3ccccc3)c3ccccc3-c3ccc(-n4c5ccccc5c5ccccc54)cc32)c1 ; Brc1cccc(C2(c3ccccc3)c3cc(-n4c5ccccc5c5ccccc54)ccc3-c3c2ccc2ccccc32)c1 ; Brc1ccc2c(c1)C1(c3ccccc3-c3ccccc31)c1cc(-n3c4ccccc4c4ccccc43)ccc1-2]

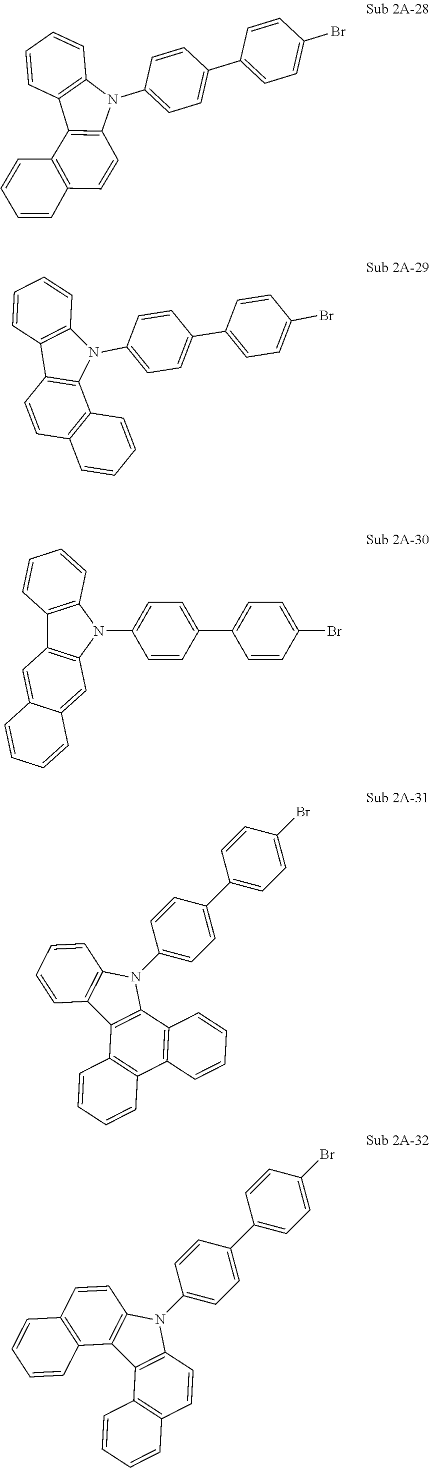 [SMILES: Brc1ccc(-c2ccc(-n3c4ccccc4c4c5ccccc5ccc43)cc2)cc1 ; Brc1ccc(-c2ccc(-n3c4ccccc4c4ccc5ccccc5c43)cc2)cc1 ; Brc1ccc(-c2ccc(-n3c4ccccc4c4cc5ccccc5cc43)cc2)cc1 ; Brc1ccc(-c2ccc(-n3c4ccccc4c4c5ccccc5c5ccccc5c43)cc2)cc1 ; Brc1ccc(-c2ccc(-n3c4ccc5ccccc5c4c4c5ccccc5ccc43)cc2)cc1]

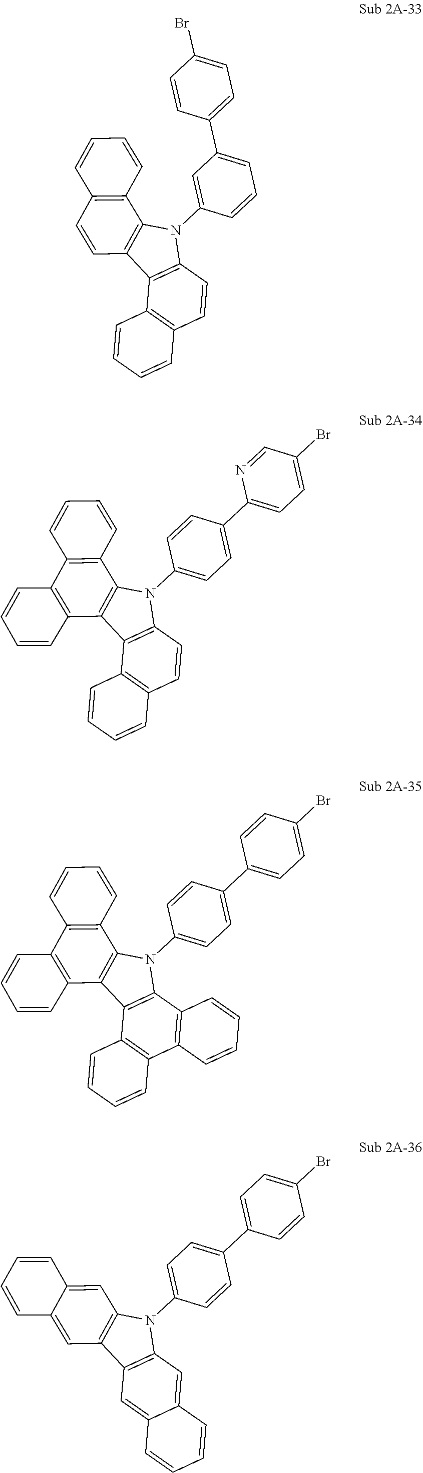 [SMILES: Brc1ccc(-c2cccc(-n3c4ccc5ccccc5c4c4ccc5ccccc5c43)c2)cc1 ; Brc1ccc(-c2ccc(-n3c4ccc5ccccc5c4c4c5ccccc5c5ccccc5c43)cc2)nc1 ; Brc1ccc(-c2ccc(-n3c4c5ccccc5c5ccccc5c4c4c5ccccc5c5ccccc5c43)cc2)cc1 ; Brc1ccc(-c2ccc(-n3c4cc5ccccc5cc4c4cc5ccccc5cc43)cc2)cc1]

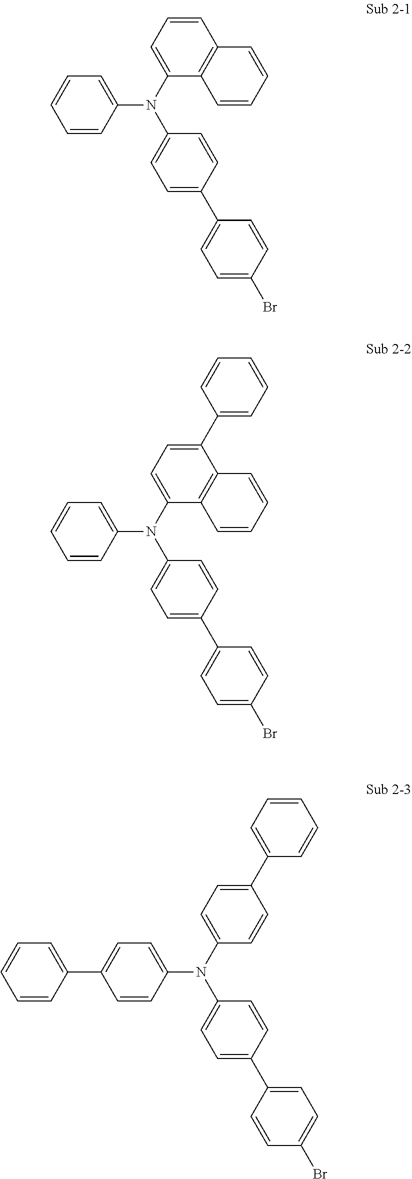 [SMILES: Brc1ccc(-c2ccc(N(c3ccccc3)c3cccc4ccccc34)cc2)cc1 ; Brc1ccc(-c2ccc(N(c3ccccc3)c3ccc(-c4ccccc4)c4ccccc34)cc2)cc1 ; Brc1ccc(-c2ccc(N(c3ccc(-c4ccccc4)cc3)c3ccc(-c4ccccc4)cc3)cc2)cc1]

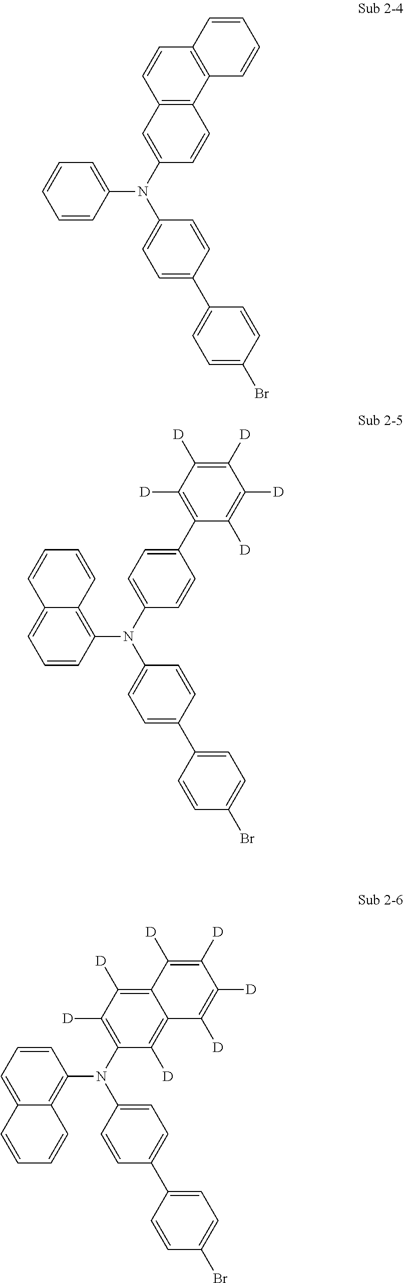

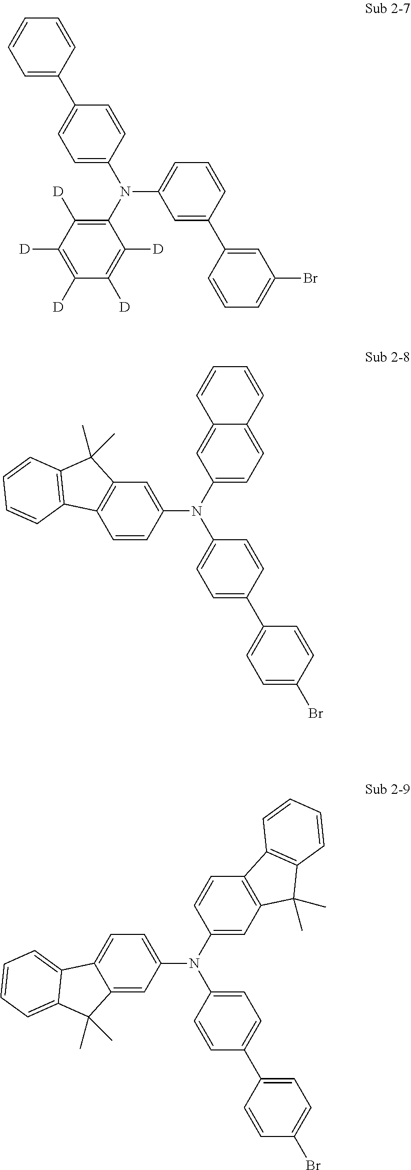 [SMILES: [2H]c1c([2H])c([2H])c(N(c2ccc(-c3ccccc3)cc2)c2cccc(-c3cccc(Br)c3)c2)c([2H])c1[2H] ; CC1(C)c2ccccc2-c2ccc(N(c3ccc(-c4ccc(Br)cc4)cc3)c3ccc4ccccc4c3)cc21 ; CC1(C)c2ccccc2-c2ccc(N(c3ccc(-c4ccc(Br)cc4)cc3)c3ccc4c(c3)C(C)(C)c3ccccc3-4)cc21]

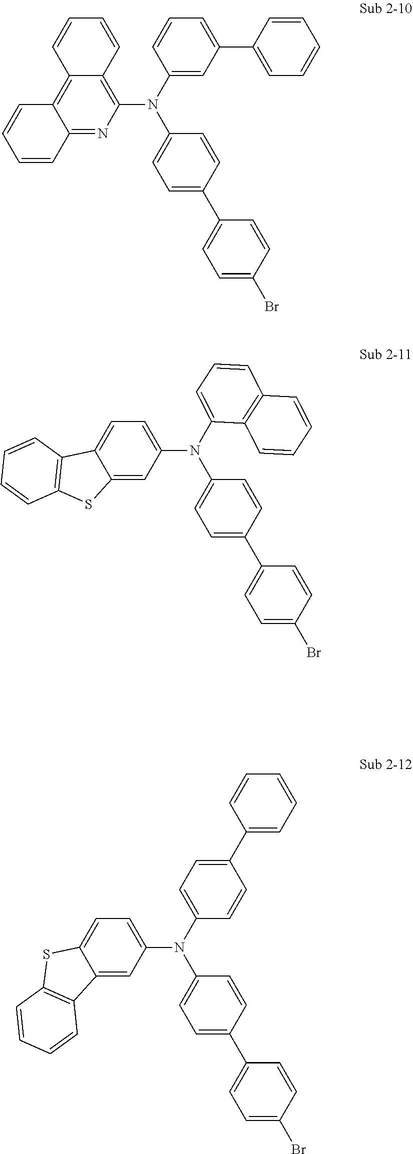

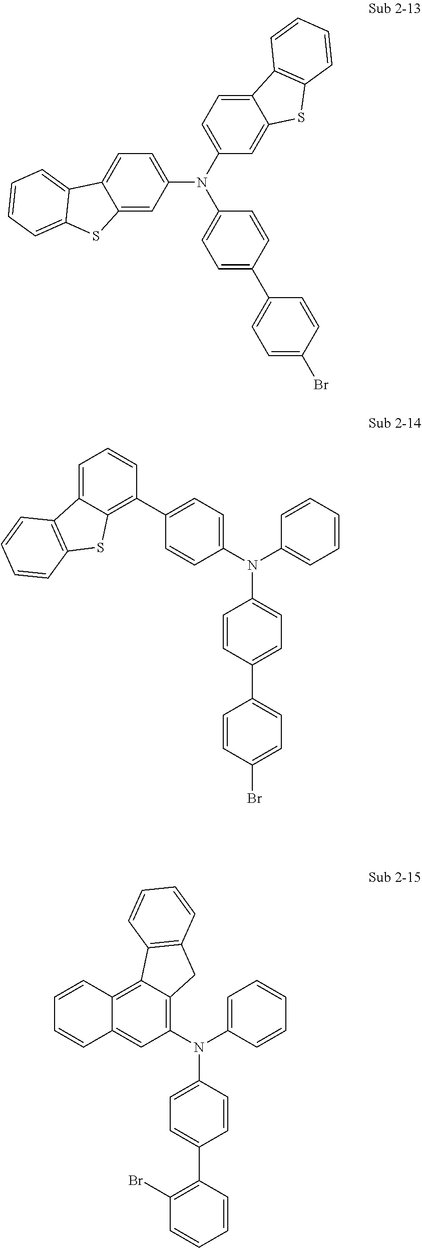 [SMILES: Brc1ccc(-c2ccc(N(c3ccc4c(c3)sc3ccccc34)c3ccc4c(c3)sc3ccccc34)cc2)cc1 ; Brc1ccc(-c2ccc(N(c3ccccc3)c3ccc(-c4cccc5c4sc4ccccc45)cc3)cc2)cc1 ; Brc1ccccc1-c1ccc(N(c2ccccc2)c2cc3ccccc3c3c2Cc2ccccc2-3)cc1]

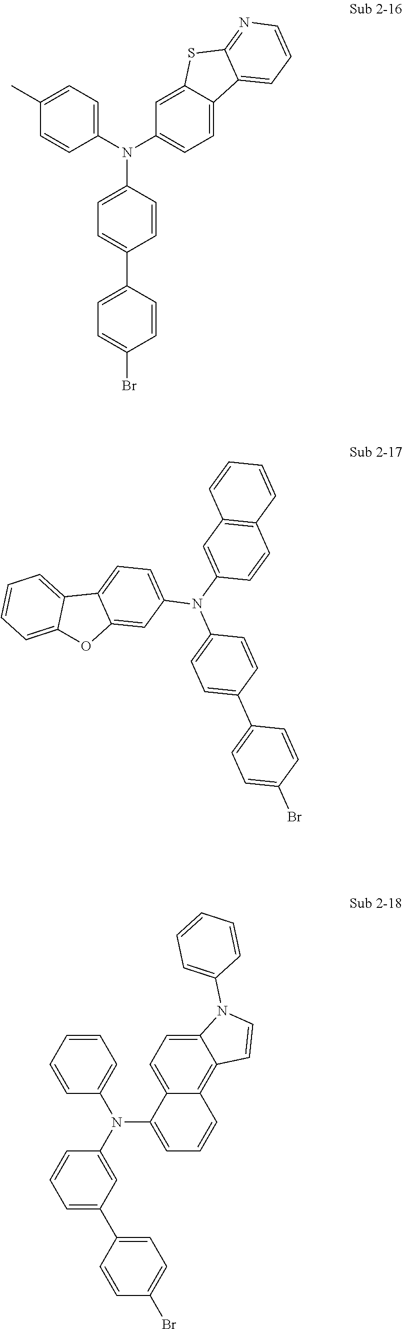 [SMILES: Cc1ccc(N(c2ccc(-c3ccc(Br)cc3)cc2)c2ccc3c(c2)sc2ncccc23)cc1 ; Brc1ccc(-c2ccc(N(c3ccc4ccccc4c3)c3ccc4c(c3)oc3ccccc34)cc2)cc1 ; Brc1ccc(-c2cccc(N(c3ccccc3)c3cccc4c3ccc3c4ccn3-c3ccccc3)c2)cc1]

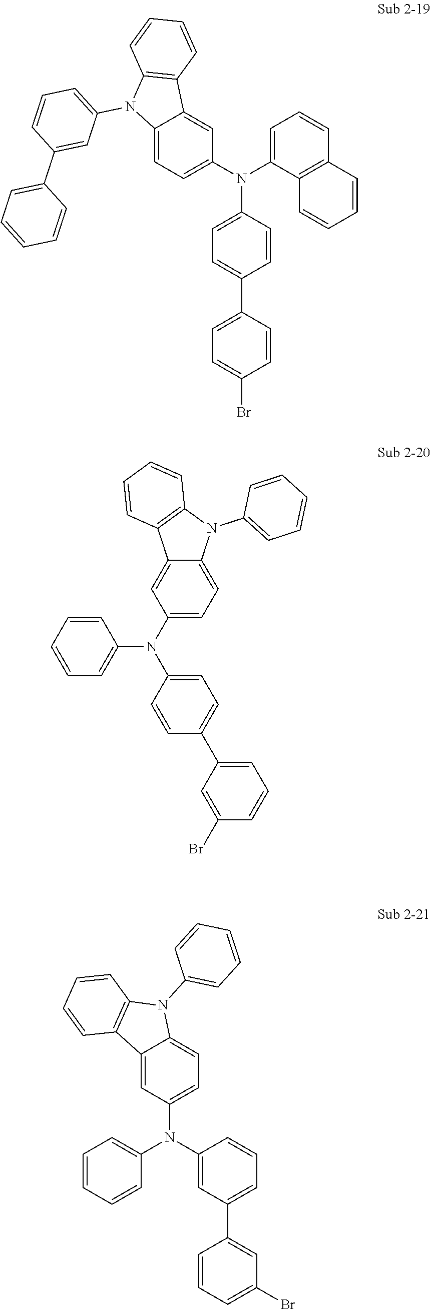 [SMILES: Brc1ccc(-c2ccc(N(c3ccc4c(c3)c3ccccc3n4-c3cccc(-c4ccccc4)c3)c3cccc4ccccc34)cc2)cc1 ; Brc1cccc(-c2ccc(N(c3ccccc3)c3ccc4c(c3)c3ccccc3n4-c3ccccc3)cc2)c1 ; Brc1cccc(-c2cccc(N(c3ccccc3)c3ccc4c(c3)c3ccccc3n4-c3ccccc3)c2)c1]

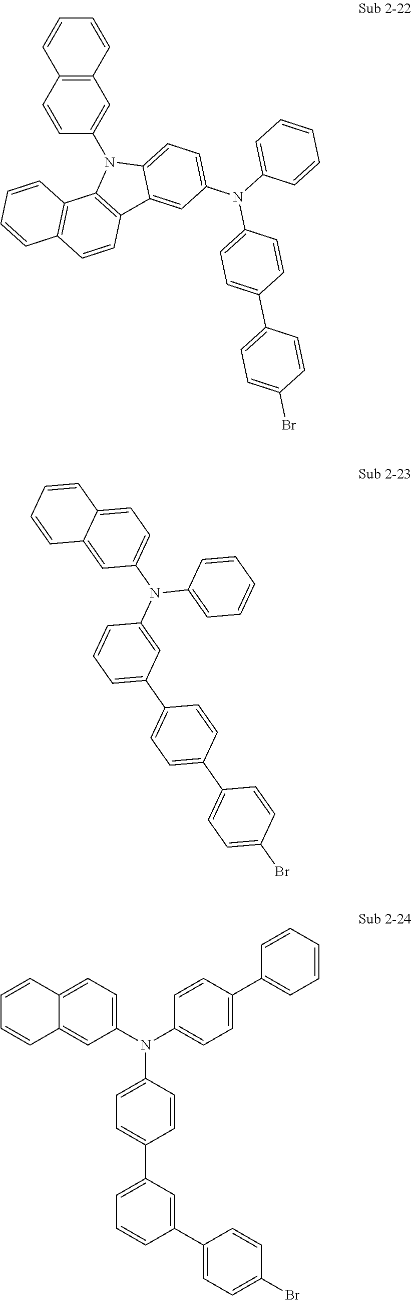 [SMILES: Brc1ccc(-c2ccc(N(c3ccccc3)c3ccc4c(c3)c3ccc5ccccc5c3n4-c3ccc4ccccc4c3)cc2)cc1 ; Brc1ccc(-c2ccc(-c3cccc(N(c4ccccc4)c4ccc5ccccc5c4)c3)cc2)cc1 ; Brc1ccc(-c2cccc(-c3ccc(N(c4ccc(-c5ccccc5)cc4)c4ccc5ccccc5c4)cc3)c2)cc1]

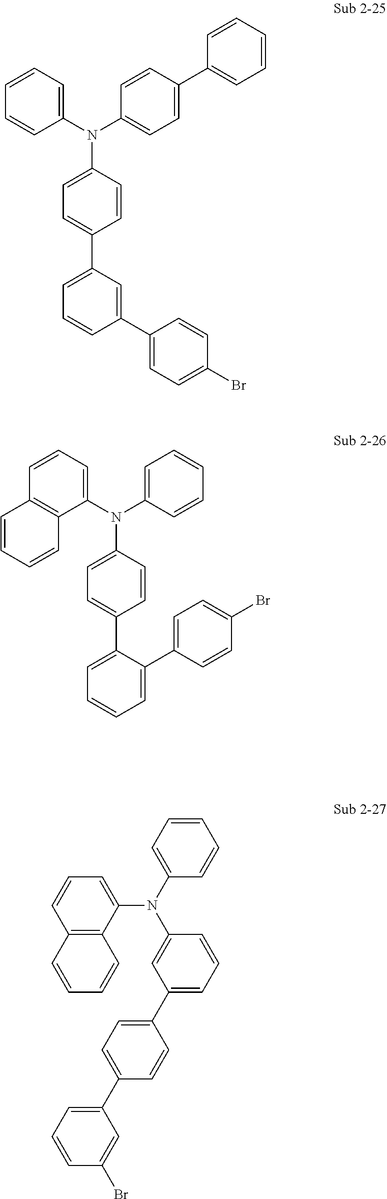 [SMILES: Brc1ccc(-c2cccc(-c3ccc(N(c4ccccc4)c4ccc(-c5ccccc5)cc4)cc3)c2)cc1 ; Brc1ccc(-c2ccccc2-c2ccc(N(c3ccccc3)c3cccc4ccccc34)cc2)cc1 ; Brc1cccc(-c2ccc(-c3cccc(N(c4ccccc4)c4cccc5ccccc45)c3)cc2)c1]

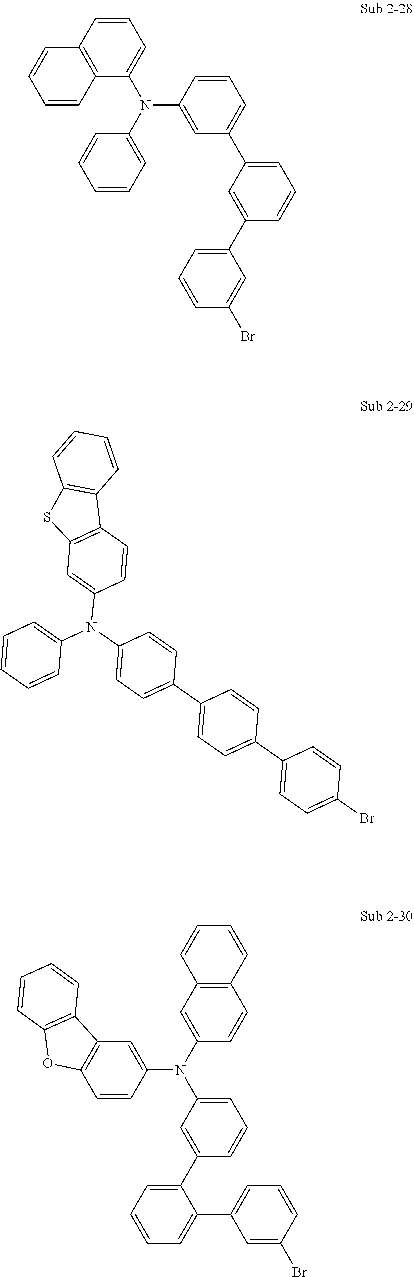 [SMILES: Brc1cccc(-c2cccc(-c3cccc(N(c4ccccc4)c4cccc5ccccc45)c3)c2)c1 ; Brc1ccc(-c2ccc(-c3ccc(N(c4ccccc4)c4ccc5c(c4)sc4ccccc45)cc3)cc2)cc1 ; Brc1cccc(-c2ccccc2-c2cccc(N(c3ccc4ccccc4c3)c3ccc4oc5ccccc5c4c3)c2)c1]

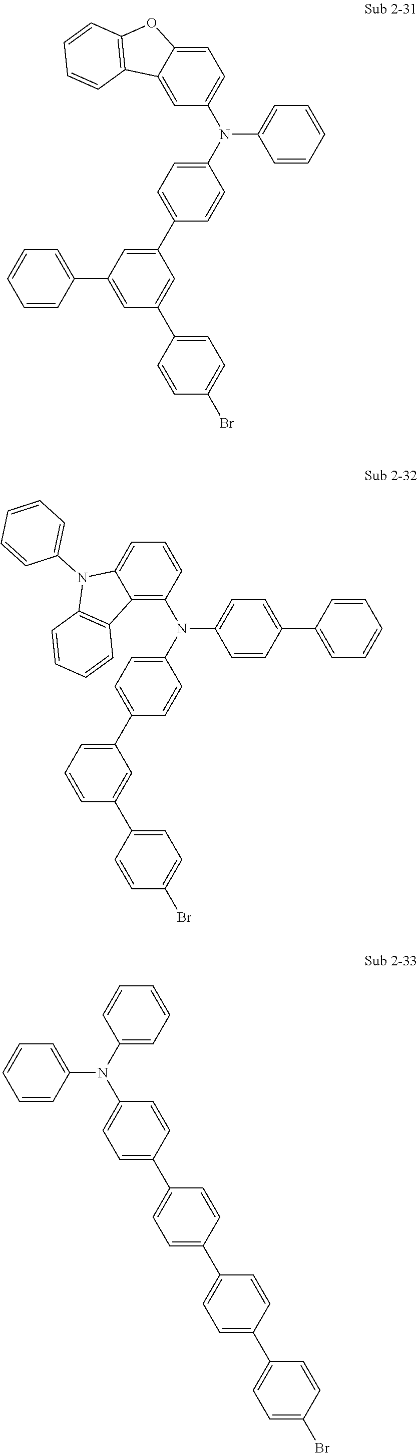 [SMILES: Brc1ccc(-c2cc(-c3ccccc3)cc(-c3ccc(N(c4ccccc4)c4ccc5oc6ccccc6c5c4)cc3)c2)cc1 ; Brc1ccc(-c2cccc(-c3ccc(N(c4ccc(-c5ccccc5)cc4)c4cccc5c4c4ccccc4n5-c4ccccc4)cc3)c2)cc1 ; Brc1ccc(-c2ccc(-c3ccc(-c4ccc(N(c5ccccc5)c5ccccc5)cc4)cc3)cc2)cc1]

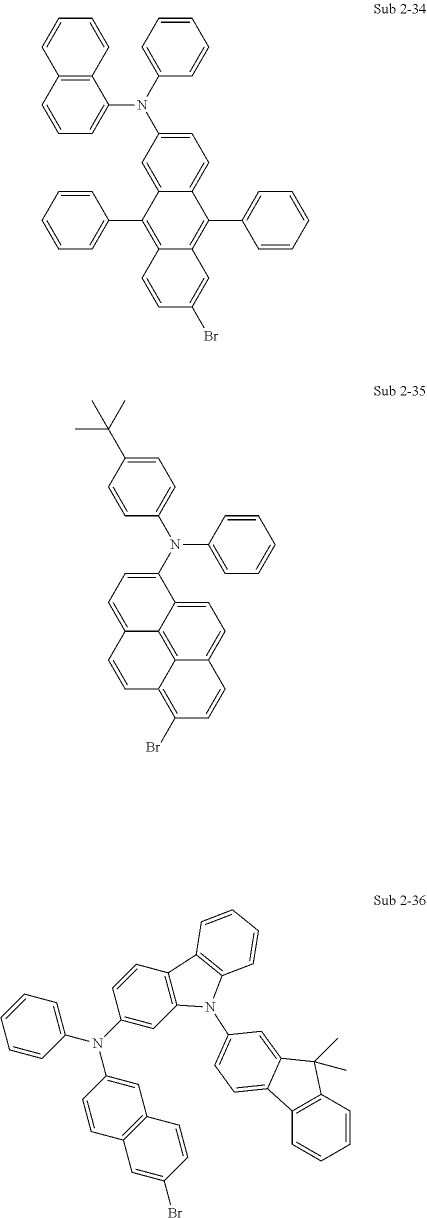 [SMILES: Brc1ccc2c(-c3ccccc3)c3cc(N(c4ccccc4)c4cccc5ccccc45)ccc3c(-c3ccccc3)c2c1 ; CC(C)(C)c1ccc(N(c2ccccc2)c2ccc3ccc4c(Br)ccc5ccc2c3c54)cc1 ; CC1(C)c2ccccc2-c2ccc(-n3c4ccccc4c4ccc(N(c5ccccc5)c5ccc6cc(Br)ccc6c5)cc43)cc21]

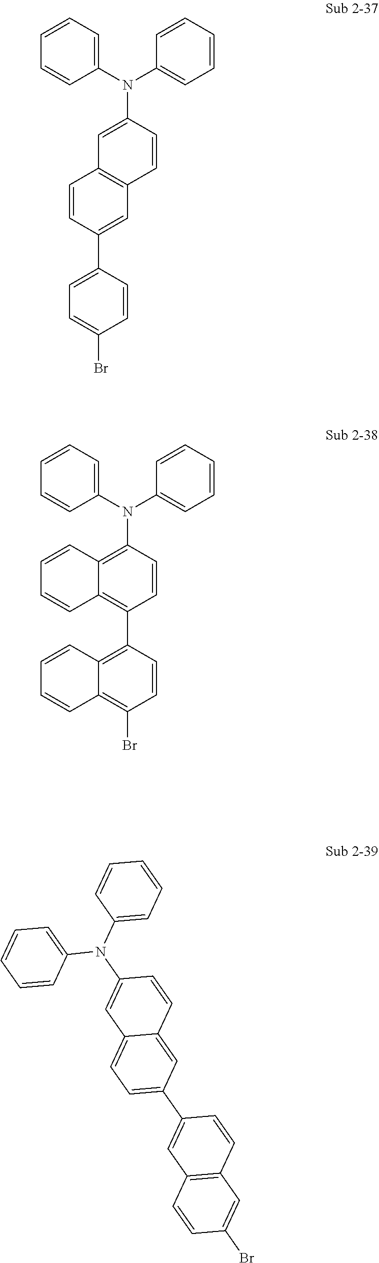 [SMILES: Brc1ccc(-c2ccc3cc(N(c4ccccc4)c4ccccc4)ccc3c2)cc1 ; Brc1ccc(-c2ccc(N(c3ccccc3)c3ccccc3)c3ccccc23)c2ccccc12 ; Brc1ccc2cc(-c3ccc4cc(N(c5ccccc5)c5ccccc5)ccc4c3)ccc2c1]

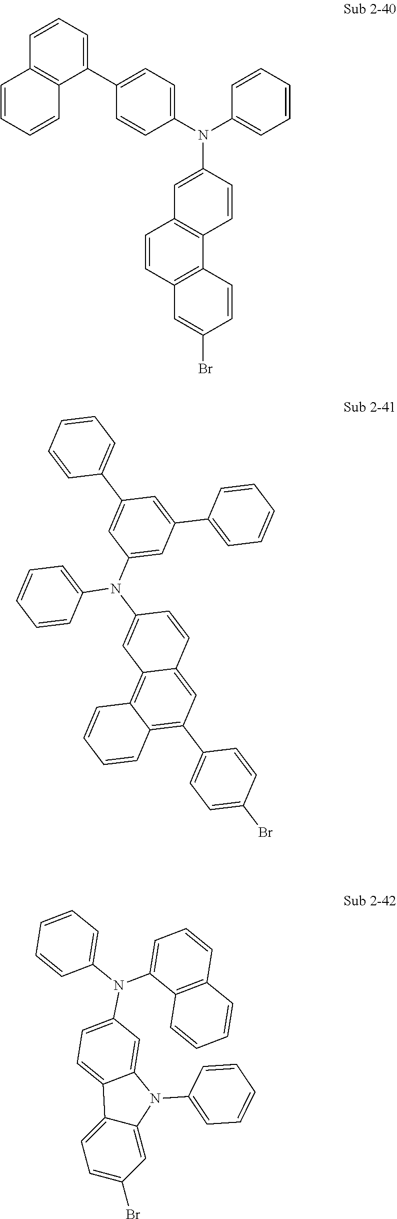 [SMILES: Brc1ccc2c(ccc3cc(N(c4ccccc4)c4ccc(-c5cccc6ccccc56)cc4)ccc32)c1 ; Brc1ccc(-c2cc3ccc(N(c4ccccc4)c4cc(-c5ccccc5)cc(-c5ccccc5)c4)cc3c3ccccc23)cc1 ; Brc1ccc2c3ccc(N(c4ccccc4)c4cccc5ccccc45)cc3n(-c3ccccc3)c2c1]

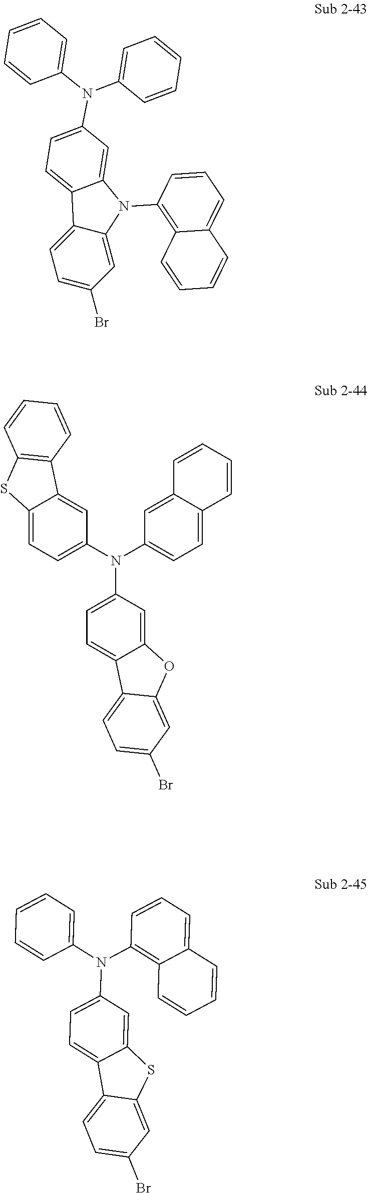 [SMILES: Brc1ccc2c3ccc(N(c4ccccc4)c4ccccc4)cc3n(-c3cccc4ccccc34)c2c1 ; Brc1ccc2c(c1)oc1cc(N(c3ccc4ccccc4c3)c3ccc4sc5ccccc5c4c3)ccc12 ; Brc1ccc2c(c1)sc1cc(N(c3ccccc3)c3cccc4ccccc34)ccc12]

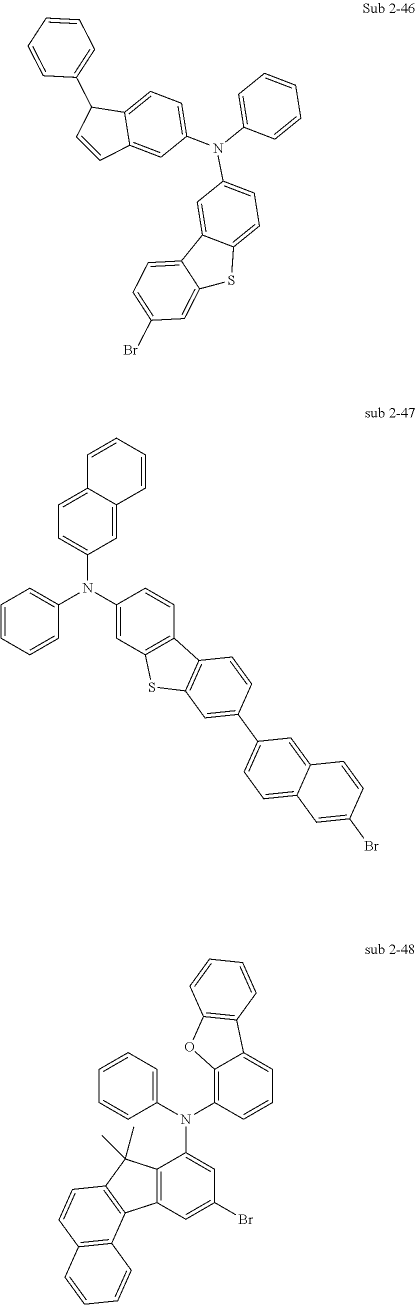 [SMILES: Brc1ccc2c(c1)sc1ccc(N(c3ccccc3)c3ccc4c(c3)C=CC4c3ccccc3)cc12 ; Brc1ccc2cc(-c3ccc4c(c3)sc3cc(N(c5ccccc5)c5ccc6ccccc6c5)ccc34)ccc2c1 ; CC1(C)c2ccc3ccccc3c2-c2cc(Br)cc(N(c3ccccc3)c3cccc4c3oc3ccccc34)c21]

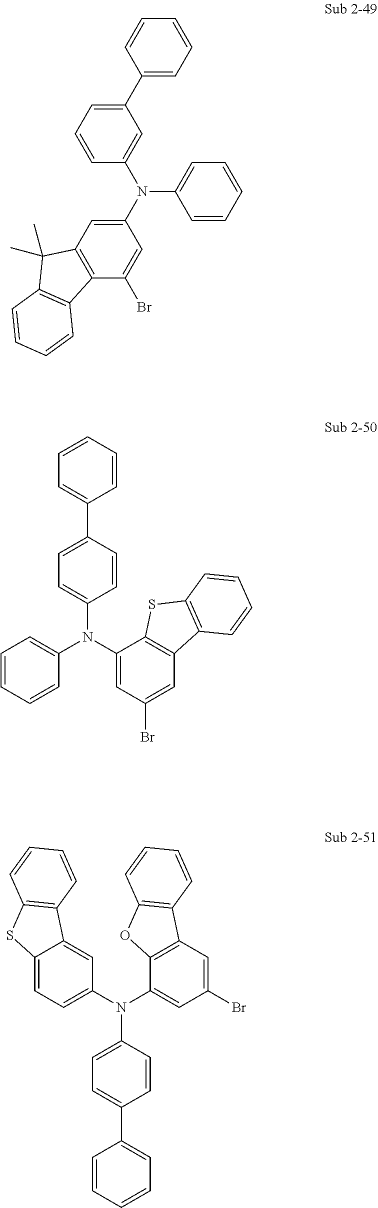 [SMILES: CC1(C)c2ccccc2-c2c(Br)cc(N(c3ccccc3)c3cccc(-c4ccccc4)c3)cc21 ; Brc1cc(N(c2ccccc2)c2ccc(-c3ccccc3)cc2)c2sc3ccccc3c2c1 ; Brc1cc(N(c2ccc(-c3ccccc3)cc2)c2ccc3sc4ccccc4c3c2)c2oc3ccccc3c2c1]

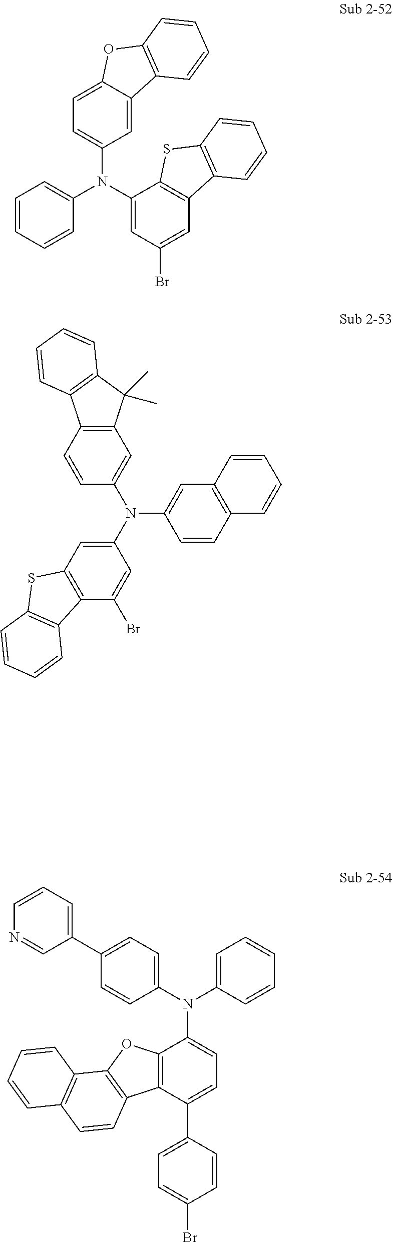 [SMILES: Brc1cc(N(c2ccccc2)c2ccc3oc4ccccc4c3c2)c2sc3ccccc3c2c1 ; CC1(C)c2ccccc2-c2ccc(N(c3ccc4ccccc4c3)c3cc(Br)c4c(c3)sc3ccccc34)cc21 ; Brc1ccc(-c2ccc(N(c3ccccc3)c3ccc(-c4cccnc4)cc3)c3oc4c5ccccc5ccc4c23)cc1]

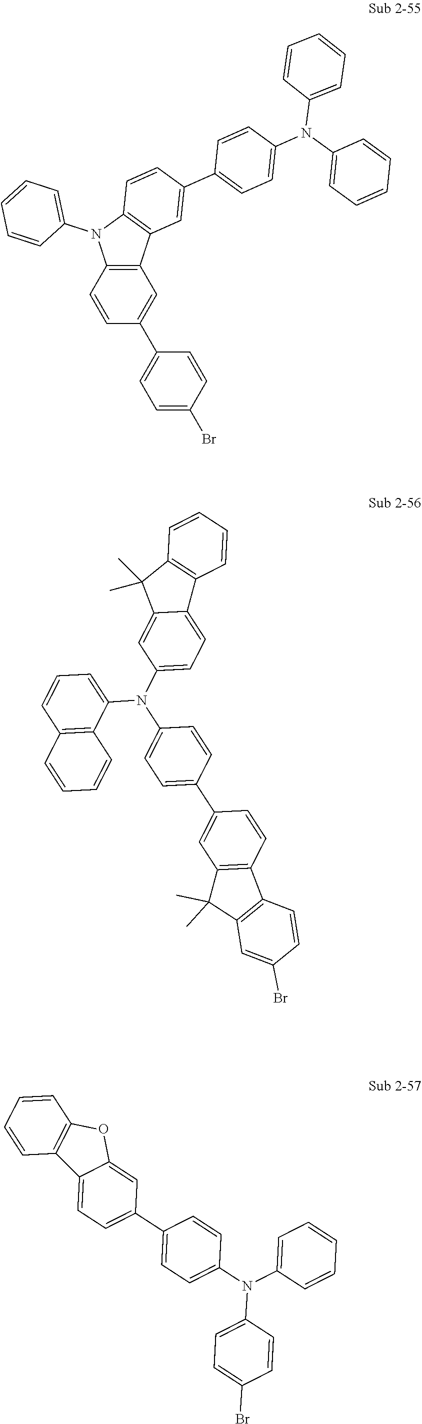 [SMILES: Brc1ccc(-c2ccc3c(c2)c2cc(-c4ccc(N(c5ccccc5)c5ccccc5)cc4)ccc2n3-c2ccccc2)cc1 ; CC1(C)c2cc(Br)ccc2-c2ccc(-c3ccc(N(c4ccc5c(c4)C(C)(C)c4ccccc4-5)c4cccc5ccccc45)cc3)cc21 ; Brc1ccc(N(c2ccccc2)c2ccc(-c3ccc4c(c3)oc3ccccc34)cc2)cc1]

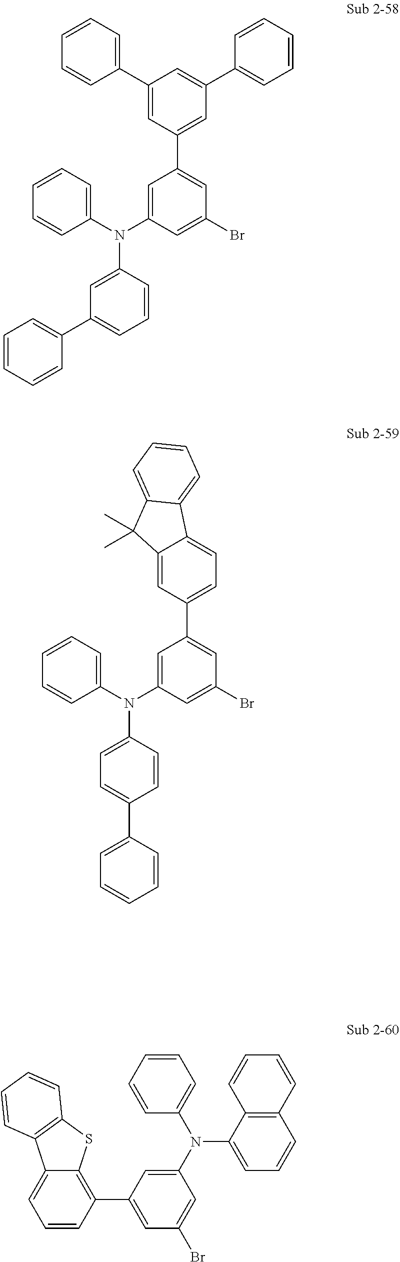 [SMILES: Brc1cc(-c2cc(-c3ccccc3)cc(-c3ccccc3)c2)cc(N(c2ccccc2)c2cccc(-c3ccccc3)c2)c1 ; CC1(C)c2ccccc2-c2ccc(-c3cc(Br)cc(N(c4ccccc4)c4ccc(-c5ccccc5)cc4)c3)cc21 ; Brc1cc(-c2cccc3c2sc2ccccc23)cc(N(c2ccccc2)c2cccc3ccccc23)c1]

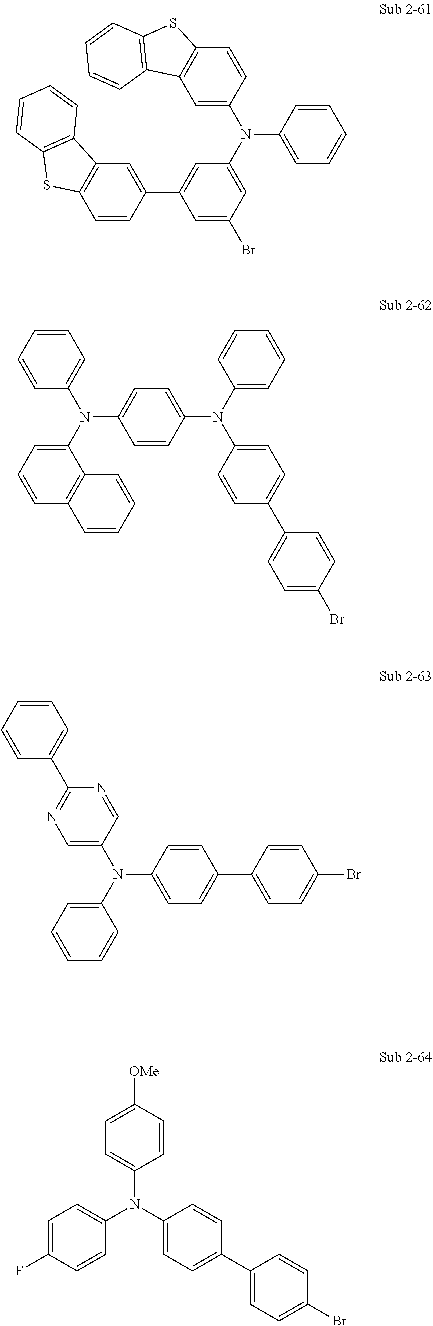 [SMILES: Brc1cc(-c2ccc3sc4ccccc4c3c2)cc(N(c2ccccc2)c2ccc3sc4ccccc4c3c2)c1 ; Brc1ccc(-c2ccc(N(c3ccccc3)c3ccc(N(c4ccccc4)c4cccc5ccccc45)cc3)cc2)cc1 ; Brc1ccc(-c2ccc(N(c3ccccc3)c3cnc(-c4ccccc4)nc3)cc2)cc1 ; COc1ccc(N(c2ccc(F)cc2)c2ccc(-c3ccc(Br)cc3)cc2)cc1]

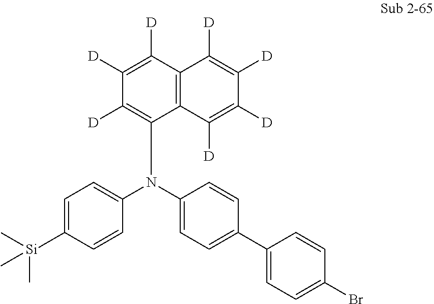 [SMILES: [2H]c1c([2H])c([2H])c2c(N(c3ccc(-c4ccc(Br)cc4)cc3)c3ccc([Si](C)(C)C)cc3)c([2H])c([2H])c([2H])c2c1[2H]]

TABLE 2

| compound | FD-MS | compound | FD-MS |
|---|---|---|---|
| Sub 2A-1 | m/z = 397.05(C₂₄H₁₆BrN = 398.29) | Sub 2A-2 | m/z = 474.07(C₂₉H₁₉BrN₂ = 475.38) |
| Sub 2A-3 | m/z = 397.05(C₂₄H₁₆BrN = 398.29) | Sub 2A-4 | m/z = 397.05(C₂₄H₁₆BrN = 398.29) |
| Sub 2A-5 | m/z = 397.05(C₂₄H₁₆BrN = 398.29) | Sub 2A-6 | m/z = 397.05(C₂₄H₁₆BrN = 398.29) |
| Sub 2A-7 | m/z = 397.05(C₂₄H₁₆BrN = 398.29) | Sub 2A-8 | m/z = 474.96(C₂₄H₁₅Br₂N = 477.20) |
| Sub 2A-9 | m/z = 473.08(C₃₀H₂₀BrN = 474.39) | Sub 2A-10 | m/z = 478.11(C₃₀H₁₅D₅BrN = 479.43) |
| Sub 2A-11 | m/z = 550.10(C₃₅H₂₃BrN₂ = 551.49) | Sub 2A-12 | m/z = 579.07(C₃₆H₂₂BrNS = 580.54) |
| Sub 2A-13 | m/z = 638.14(C₄₂H₂₇BrN₂ = 639.58) | Sub 2A-14 | m/z = 321.02(C₁₈H₁₂BrN = 322.20) |
| Sub 2A-15 | m/z = 447.06(C₂₈H₁₈BrN = 448.35) | Sub 2A-16 | m/z = 473.08(C₃₀H₂₀BrN = 474.40) |
| Sub 2A-17 | m/z = 493.14(C₃₁H₂₈BrN = 494.48) | Sub 2A-18 | m/z = 527.03(C₃₂H₁₈BrNS = 528.46) |
| Sub 2A-19 | m/z = 411.03(C₂₄H₁₄BrNO = 412.28) | Sub 2A-20 | m/z = 427.00(C₂₄H₁₄BrNS = 428.34) |
| Sub 2A-21 | m/z = 411.03 (C₂₄H₁₄BrNO = 412.28) | Sub 2A-22 | m/z = 437.08(C₂₇H₂₀BrN = 438.36) |
| Sub 2A-23 | m/z = 561.11(C₃₇H₂₄BrN = 562.50) | Sub 2A-24 | m/z = 487.09(C₃₁H₂₂BrN = 488.42) |
| Sub 2A-25 | m/z = 561.11 (C₃₇H₂₄BrN = 562.50) | Sub 2A-26 | m/z = 611.12(C₄₁H₂₆BrN = 612.56) |
| Sub 2A-27 | m/z = 559.09(C₃₇H₂₂BrN = 560.48) | Sub 2A-28 | m/z = 447.06(C₂₈H₁₈BrN = 448.35) |
| Sub 2A-29 | m/z = 447.06(C₂₈H₁₈BrN = 448.35) | Sub 2A-30 | m/z = 447.06(C₂₈H₁₈BrN = 448.35) |
| Sub 2A-31 | m/z = 497.08(C₃₂H₂₀BrN = 498.41) | Sub 2A-32 | m/z = 497.08(C₃₂H₂₀BrN = 498.41) |
| Sub 2A-33 | m/z = 497.08(C₃₂H₂₀BrN = 498.41) | Sub 2A-34 | m/z = 548.09(C₃₅H₂₁BrN₂ = 549.46) |
| Sub 2A-35 | m/z = 597.11(C₄₀H₂₄BrN = 598.53) | Sub 2A-36 | m/z = 497.08(C₃₂H₂₀BrN = 498.41) |
| Sub 2-1 | m/z = 449.08(C₂₈H₂₀BrN = 450.37) | Sub 2-2 | m/z = 525.11(C₃₄H₂₄BrN = 526.48) |
| Sub 2-3 | m/z = 551.12(C₃₆H₂₆BrN = 552.50) | Sub 2-4 | m/z = 499.09(C₃₂H₂₂BrN = 500.43) |
| Sub 2-5 | m/z = 530.14(C₃₄H₁₉D₅BrN = 531.51) | Sub 2-6 | m/z = 506.14(C₃₂H₁₅D₇BrN = 507.48) |
| Sub 2-7 | m/z = 480.12(C₃₀H₁₇D₅BrN = 481.45) | Sub 2-8 | m/z = 565.14(C₃₇H₂₈BrN = 566.54) |
| Sub 2-9 | m/z = 631.19(C₄₂H₃₄BrN = 632.65) | Sub 2-10 | m/z = 576.12(C₃₇H₂₅BrN₂ = 577.53) |
| Sub 2-11 | m/z = 555.07(C₃₄H₂₂BrNS = 556.51) | Sub 2-12 | m/z = 581.08(C₃₆H₂₄BrNS = 582.55) |
| Sub 2-13 | m/z = 611.04(C₃₆H₂₂BrNS₂ = 612.60) | Sub 2-14 | m/z = 581.08(C₃₆H₂₄BrNS = 582.55) |
| Sub 2-15 | m/z = 704.28(C₅₂H₃₆N₂O = 704.87) | Sub 2-16 | m/z = 520.06(C₃₀H₂₁BrN₂S = 521.48) |
| Sub 2-17 | m/z = 539.09(C₃₄H₂₂BrNO = 540.45) | Sub 2-18 | m/z = 564.12(C₃₆H₂₅BrN₂ = 565.50) |
| Sub 2-19 | m/z = 690.17(C₄₆H₃₁BrN₂ = 691.67) | Sub 2-20 | m/z = 657.11(C₄₂H₂₈BrNS = 658.65) |
| Sub 2-21 | m/z = 564.12(C₃₆H₂₅BrN₂ = 565.50) | Sub 2-22 | m/z = 664.15(C₄₄H₂₉BrN₂ = 665.63) |
| Sub 2-23 | m/z = 525.11(C₃₄H₂₄BrN = 526.47) | Sub 2-24 | m/z = 601.14(C₄₀H₂₈BrN = 602.58) |
| Sub 2-25 | m/z = 551.12(C₃₆H₂₆BrN = 552.52) | Sub 2-26 | m/z = 525.11(C₃₄H₂₄BrN = 526.47) |
| Sub 2-27 | m/z = 525.11(C₃₄H₂₄BrN = 526.47) | Sub 2-28 | m/z = 525.11(C₃₄H₂₄BrN = 526.47) |
| Sub 2-29 | m/z = 581.08(C₃₆H₂₄BrNS = 582.55) | Sub 2-30 | m/z = 615.12(C₄₀H₂₆BrNO = 616.54) |
| Sub 2-31 | m/z = 641.14 (C₄₂H₂₈BrNO = 642.58) | Sub 2-32 | m/z = 716.18(C₄₈H₃₃BrN₂ = 717.71) |
| Sub 2-33 | m/z = 475.09(C₃₀H₂₂BrN = 476.41) | Sub 2-34 | m/z = 625.14(C₄₂H₂₈BrN = 626.58) |
| Sub 2-35 | m/z = 503.12(C₃₂H₂₆BrN = 504.46) | Sub 2-36 | m/z = 538.10(C₃₄H₂₃BrN₂ = 539.46) |
| Sub 2-37 | m/z = 449.08(C₂₈H₂₀BrN = 450.38) | Sub 2-38 | m/z = 499.09(C₃₂H₂₂BrN = 500.43) |
| Sub 2-39 | m/z = 499.09(C₃₂H₂₂BrN = 500.43) | Sub 2-40 | m/z = 549.11(C₃₆H₂₄BrN = 550.50) |
| Sub 2-41 | m/z = 651.16(C₄₄H₃₀BrN = 652.64) | Sub 2-42 | m/z = 538.10(C₃₄H₂₃BrN₂ = 539.46) |
| Sub 2-43 | m/z = 538.10(C₃₄H₂₃BrN₂ = 539.46) | Sub 2-44 | m/z = 569.04(C₃₄H₂₀BrNOS = 570.50) |
| Sub 2-45 | m/z = 479.03(C₂₈H₁₈BrNS = 480.42) | Sub 2-46 | m/z = 544.06(C₃₂H₂₁BrN₂S = 545.49) |
| Sub 2-47 | m/z = 605.08(C₃₈H₂₄BrNS = 606.57) | Sub 2-48 | m/z = 579.12(C₃₇H₂₆BrNO = 580.53) |
| Sub 2-49 | m/z = 515.12(C₃₃H₂₆BrN = 516.48) | Sub 2-50 | m/z = 505.05(C₃₀H₂₀BrNS = 506.46) |
| Sub 2-51 | m/z = 559.06(C₃₆H₂₂BrNOS = 596.54) | Sub 2-52 | m/z = 519.03(C₃₀H₁₈BrNOS = 520.44) |
| Sub 2-53 | m/z = 595.10(C₃₇H₂₆BrNS = 596.59) | Sub 2-54 | m/z = 612.12(C₃₉H₂₅BrN₂O = 617.55) |
| Sub 2-55 | m/z = 640.15(C₄₂H₂₉BrN₂ = 641.61) | Sub 2-56 | m/z = 681.20(C₄₆H₃₆BrN = 682.71) |
| Sub 2-57 | m/z = 489.07(C₃₀H₂₀BrNO = 490.40) | Sub 2-58 | m/z = 627.16(C₄₂H₃₀BrN = 628.61) |
| Sub 2-59 | m/z = 591.16(C₃₉H₃₀BrN = 592.58) | Sub 2-60 | m/z = 555.07(C₃₄H₂₂BrNS = 556.52) |
| Sub 2-61 | m/z = 611.04(C₃₆H₂₂BrNS₂ = 612.60) | Sub 2-62 | m/z = 616.15(C₄₀H₂₉BrN₂ = 617.59) |
| Sub 2-63 | m/z = 477.08(C₂₈H₂₀BrN₃ = 478.39) | Sub 2-64 | m/z = 447.06(C₂₅H₁₉BrFNO = 448.34) |
| Sub 2-65 | m/z = 528.16(C₃₁H₂₁D₅BrNSi = 529.60) | | |

### Synthesis Example of Final Products 1

Sub 2 or Sub 2A (1 eq.) was dissolved in toluene in a round bottom flask, and Sub 1 (1.1 eq.), Pd₂(dba)₃ (0.03 eq.), P(t-Bu)₃ (0.1 eq.), NaOt-Bu (3 eq.) were added and stirred at 100° C. When the reaction was complete, the reaction mixture was extracted with CH₂Cl₂ and water. The organic layer was dried over MgSO₄ and concentrated. The resulting compound was separated by silicagel column chromatography and recrystallization to obtain the Final products.

### Synthesis of 1-3

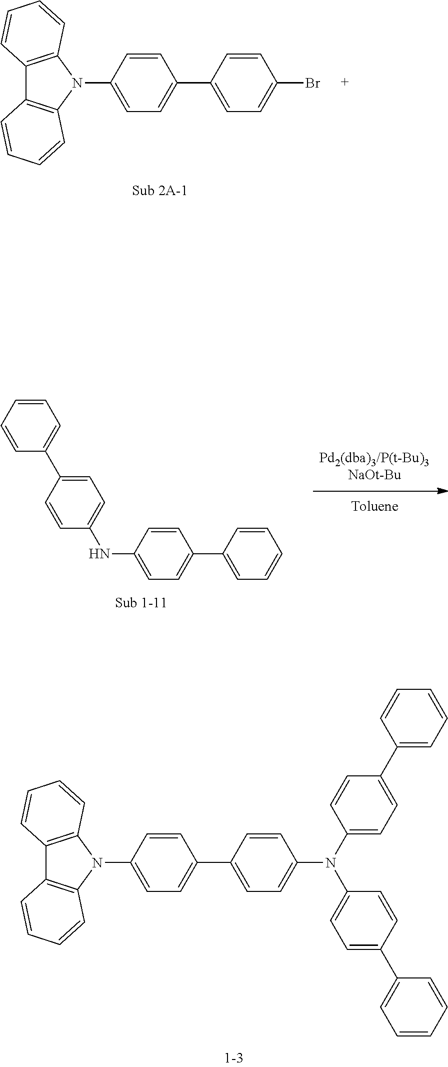 [SMILES: Brc1ccc(-c2ccc(-n3c4ccccc4c4ccccc43)cc2)cc1 ; c1ccc(-c2ccc(Nc3ccc(-c4ccccc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

Sub 2A-1 (10 g, 25.11 mmol) was dissolved in toluene (264 ml) in a round bottom flask, and Sub 1-11 (8.88 g, 27.62 mmol), Pd₂(dba)₃ (0.69 g, 0.75 mmol), P(t-Bu)₃ (0.51 g, 2.51 mmol), NaOt-Bu (7.24 g, 75.32 mmol) were added and stirred at 100° C. When the reaction was complete, the reaction mixture was extracted with CH₂Cl₂ and water. The organic layer was dried over MgSO₄ and concentrated. The resulting compound was separated by silicagel column chromatography and recrystallization to obtain 13.31 g of product. (Yield: 83%)

### Synthesis of 1-23

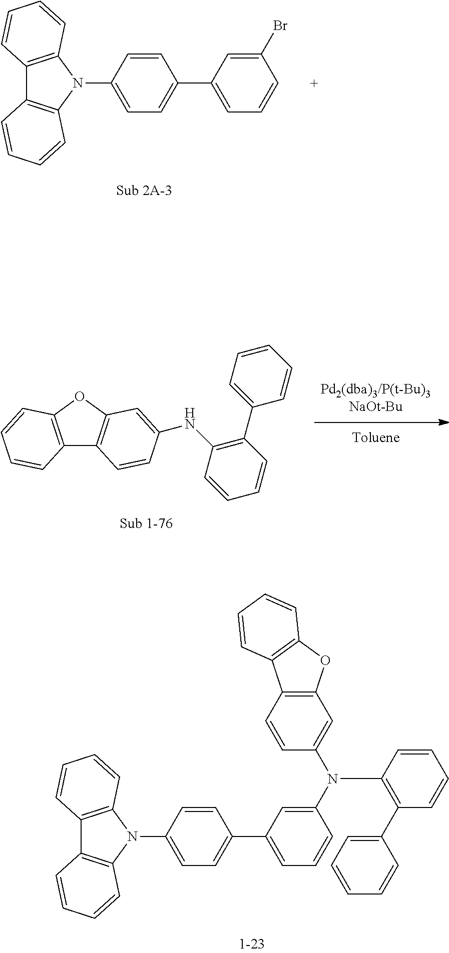 [SMILES: Brc1cccc(-c2ccc(-n3c4ccccc4c4ccccc43)cc2)c1 ; c1ccc(-c2ccccc2Nc2ccc3c(c2)oc2ccccc23)cc1 ; c1ccc(-c2ccccc2N(c2cccc(-c3ccc(-n4c5ccccc5c5ccccc54)cc3)c2)c2ccc3c(c2)oc2ccccc23)cc1]

Sub 2A-3 (10 g, 25.11 mmol), toluene (264 ml), Sub 1-76 (9.26 g, 27.62 mmol), Pd₂(dba)₃ (0.69 g, 0.75 mmol), P(t-Bu)₃ (0.51 g, 2.51 mmol), NaOt-Bu (7.24 g, 75.32 mmol) were carried out in the same manner as 1-3 to obtain 12.78 g of the product. (Yield: 78%)

### Synthesis of 1-31

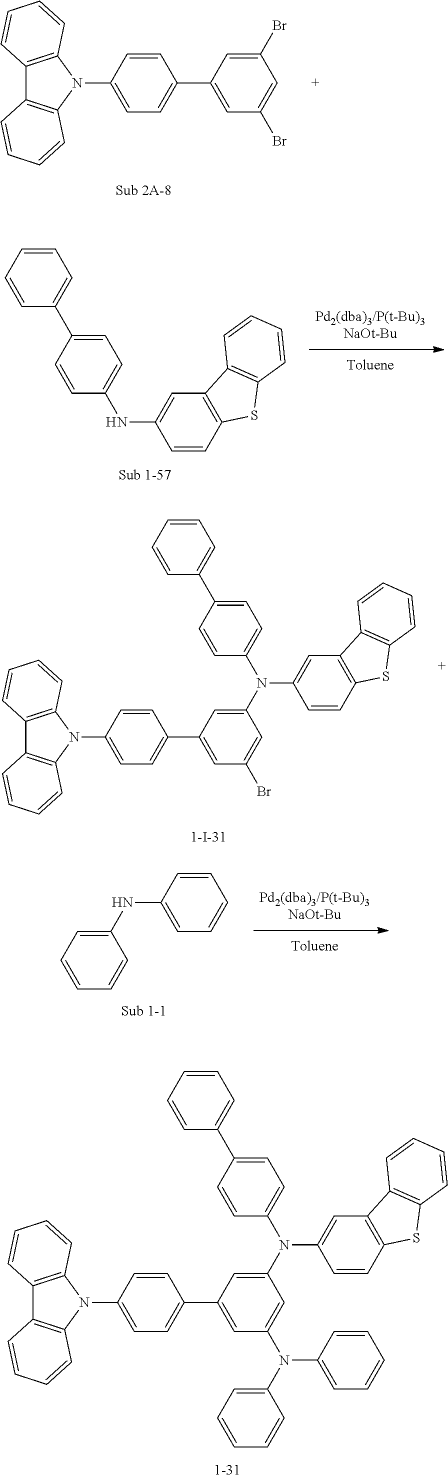 [SMILES: Brc1cc(Br)cc(-c2ccc(-n3c4ccccc4c4ccccc43)cc2)c1 ; c1ccc(-c2ccc(Nc3ccc4sc5ccccc5c4c3)cc2)cc1 ; Brc1cc(-c2ccc(-n3c4ccccc4c4ccccc43)cc2)cc(N(c2ccc(-c3ccccc3)cc2)c2ccc3sc4ccccc4c3c2)c1 ; c1ccc(Nc2ccccc2)cc1 ; c1ccc(-c2ccc(N(c3cc(-c4ccc(-n5c6ccccc6c6ccccc65)cc4)cc(N(c4ccccc4)c4ccccc4)c3)c3ccc4sc5ccccc5c4c3)cc2)cc1]

### 1) Synthesis of Intermediate 1-I-31

Sub 2A-8 (20 g, 41.91 mmol), toluene (440 ml), Sub 1-57 (16.20 g, 46.10 mmol), Pd₂(dba)₃ (0.58 g, 0.63 mmol), P(t-Bu)₃ (0.42 g, 2.10 mmol), NaOt-Bu (6.04 g, 62.87 mmol) were carried out in the same manner as 1-3 to obtain 22.25 g of the product. (Yield: 71%)

### 2) Synthesis of 1-31

1-I-31 (22.25 g, 29.76 mmol), toluene (312 ml), Sub 1-1 (5.54 g, 32.73 mmol), Pd₂(dba)₃ (0.82 g, 0.89 mmol), P(t-Bu)₃ (0.60 g, 2.98 mmol), NaOt-Bu (8.58 g, 89.27 mmol) were carried out in the same manner as 1-3 to obtain 20.65 g of the product. (Yield: 83%)

### Synthesis of 1-34

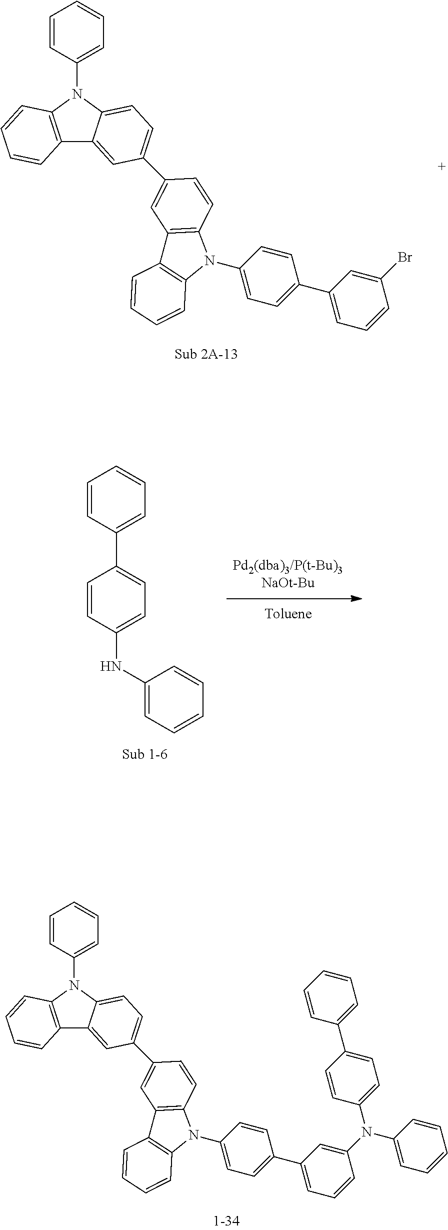 [SMILES: Brc1cccc(-c2ccc(-n3c4ccccc4c4cc(-c5ccc6c(c5)c5ccccc5n6-c5ccccc5)ccc43)cc2)c1 ; c1ccc(Nc2ccc(-c3ccccc3)cc2)cc1 ; c1ccc(-c2ccc(N(c3ccccc3)c3cccc(-c4ccc(-n5c6ccccc6c6cc(-c7ccc8c(c7)c7ccccc7n8-c7ccccc7)ccc65)cc4)c3)cc2)cc1]

Sub 2A-13 (11 g, 17.20 mmol), toluene (181 ml), Sub 1-6 (4.64 g, 18.92 mmol), Pd₂(dba)₃ (0.47 g, 0.52 mmol), P(t-Bu)₃ (0.35 g, 1.72 mmol), NaOt-Bu (4.96 g, 51.59 mmol) were carried out in the same manner as 1-3 to obtain 10.65 g of the product. (Yield: 77%)

### Synthesis of 1-43

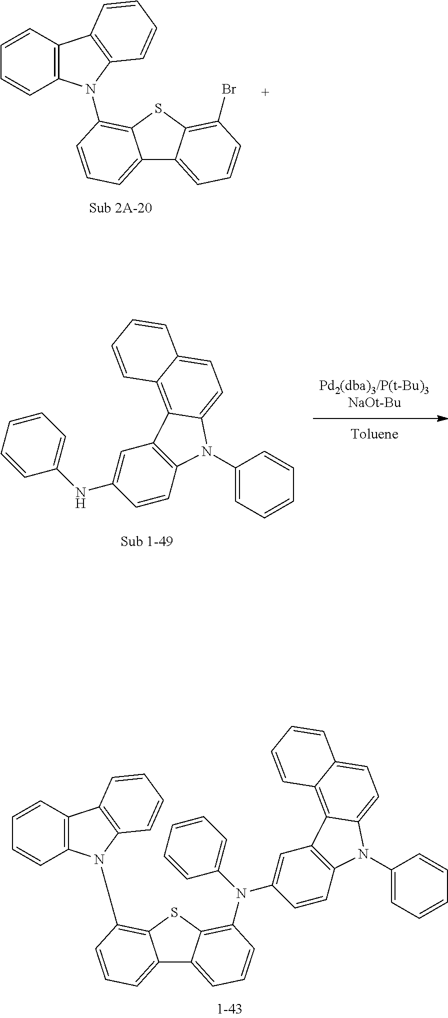 [SMILES: Brc1cccc2c1sc1c(-n3c4ccccc4c4ccccc43)cccc12 ; c1ccc(Nc2ccc3c(c2)c2c4ccccc4ccc2n3-c2ccccc2)cc1 ; c1ccc(N(c2ccc3c(c2)c2c4ccccc4ccc2n3-c2ccccc2)c2cccc3c2sc2c(-n4c5ccccc5c5ccccc54)cccc23)cc1]

Sub 2A-20 (11 g, 25.68 mmol), toluene (270 ml), Sub 1-49 (10.86 g, 28.25 mmol), Pd₂(dba)₃ (0.71 g, 0.77 mmol), P(t-Bu)₃ (0.52 g, 2.57 mmol), NaOt-Bu (7.40 g, 77.04 mmol) were carried out in the same manner as 1-3 to obtain 13.16 g of the product. (Yield: 70%)

### Synthesis of 1-45

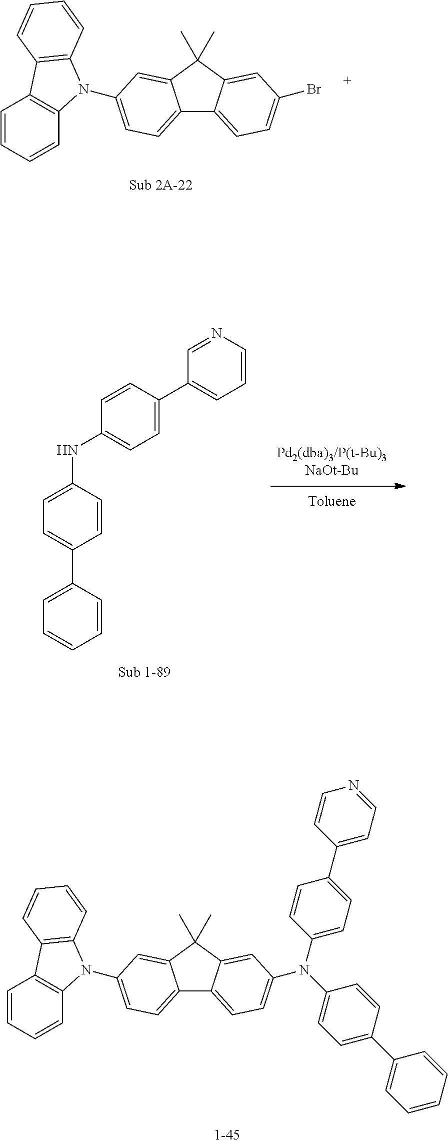 [SMILES: CC1(C)c2cc(Br)ccc2-c2ccc(-n3c4ccccc4c4ccccc43)cc21 ; c1ccc(-c2ccc(Nc3ccc(-c4cccnc4)cc3)cc2)cc1 ; CC1(C)c2cc(N(c3ccc(-c4ccccc4)cc3)c3ccc(-c4ccncc4)cc3)ccc2-c2ccc(-n3c4ccccc4c4ccccc43)cc21]

Sub 2A-22 (11 g, 25.09 mmol), toluene (263 ml), Sub 1-89 (8.90 g, 27.60 mmol), Pd₂(dba)₃ (0.69 g, 0.75 mmol), P(t-Bu)₃ (0.51 g, 2.51 mmol), NaOt-Bu (7.23 g, 75.28 mmol) were carried out in the same manner as 1-3 to obtain 11.43 g of the product. (Yield: 67%)

### Synthesis of 1-54

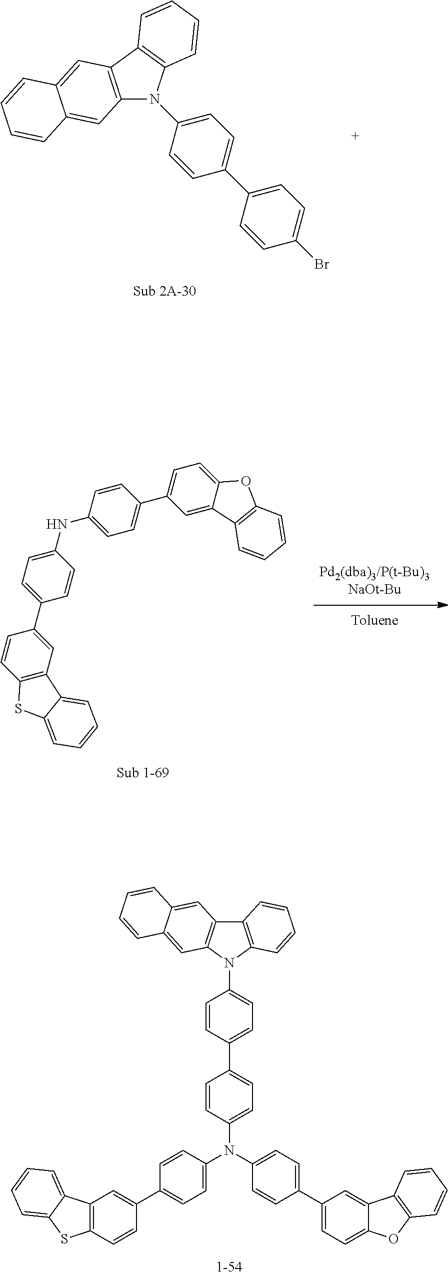 [SMILES: Brc1ccc(-c2ccc(-n3c4ccccc4c4cc5ccccc5cc43)cc2)cc1 ; c1ccc2c(c1)oc1ccc(-c3ccc(Nc4ccc(-c5ccc6sc7ccccc7c6c5)cc4)cc3)cc12 ; c1ccc2cc3c(cc2c1)c1ccccc1n3-c1ccc(-c2ccc(N(c3ccc(-c4ccc5oc6ccccc6c5c4)cc3)c3ccc(-c4ccc5sc6ccccc6c5c4)cc3)cc2)cc1]

Sub 2A-30 (7 g, 15.61 mmol), toluene (164 ml), Sub 1-69 (8.89 g, 17.17 mmol), Pd₂(dba)₃ (0.43 g, 0.47 mmol), P(t-Bu)₃ (0.32 g, 1.56 mmol), NaOt-Bu (4.50 g, 46.84 mmol) were carried out in the same manner as 1-3 to obtain 10.23 g of the product. (Yield: 74%)

### Synthesis of 1-56

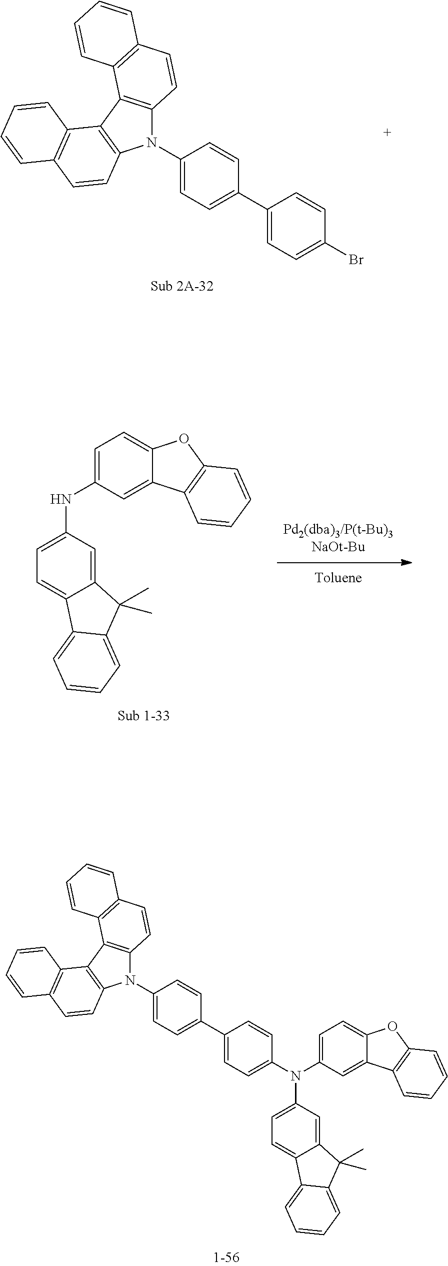 [SMILES: Brc1ccc(-c2ccc(-n3c4ccc5ccccc5c4c4c5ccccc5ccc43)cc2)cc1 ; CC1(C)c2ccccc2-c2ccc(Nc3ccc4oc5ccccc5c4c3)cc21 ; CC1(C)c2ccccc2-c2ccc(N(c3ccc(-c4ccc(-n5c6ccc7ccccc7c6c6c7ccccc7ccc65)cc4)cc3)c3ccc4oc5ccccc5c4c3)cc21]

Sub 2A-32 (9 g, 18.06 mmol), toluene (190 ml), Sub 1-33 (7.46 g, 19.86 mmol), Pd₂(dba)₃ (0.50 g, 0.54 mmol), P(t-Bu)₃ (0.37 g, 1.81 mmol), NaOt-Bu (5.21 g, 54.17 mmol) were carried out in the same manner as 1-3 to obtain 10.17 g of the product. (Yield: 71%)

### Synthesis of 2-1

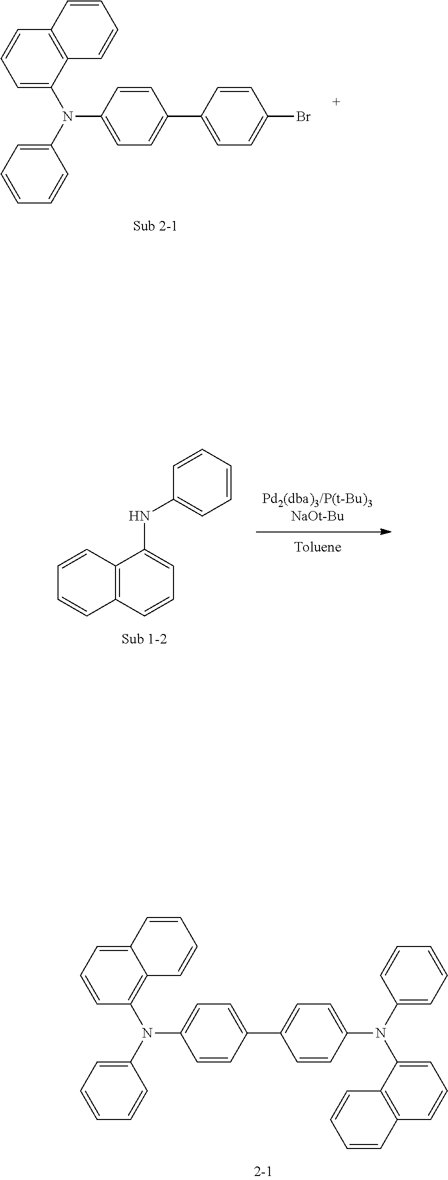 [SMILES: Brc1ccc(-c2ccc(N(c3ccccc3)c3cccc4ccccc34)cc2)cc1 ; c1ccc(Nc2cccc3ccccc23)cc1 ; c1ccc(N(c2ccc(-c3ccc(N(c4ccccc4)c4cccc5ccccc45)cc3)cc2)c2cccc3ccccc23)cc1]

Sub 2-1 (10 g, 22.20 mmol) was dissolved in toluene (233 ml) in a round bottom flask, and Sub 1-12 (5.36 g, 24.42 mmol), Pd₂(dba)₃ (0.61 g, 0.67 mmol), P(t-Bu)₃ (0.45 g, 2.22 mmol), NaOt-Bu (6.40 g, 66.61 mmol) were added and stirred at 100° C. When the reaction was complete, the reaction mixture was extracted with CH₂Cl₂ and water. The organic layer was dried over MgSO₄ and concentrated. The resulting compound was separated by silicagel column chromatography and recrystallization to obtain 10.46 g of product. (Yield: 80%)

### Synthesis of 2-10

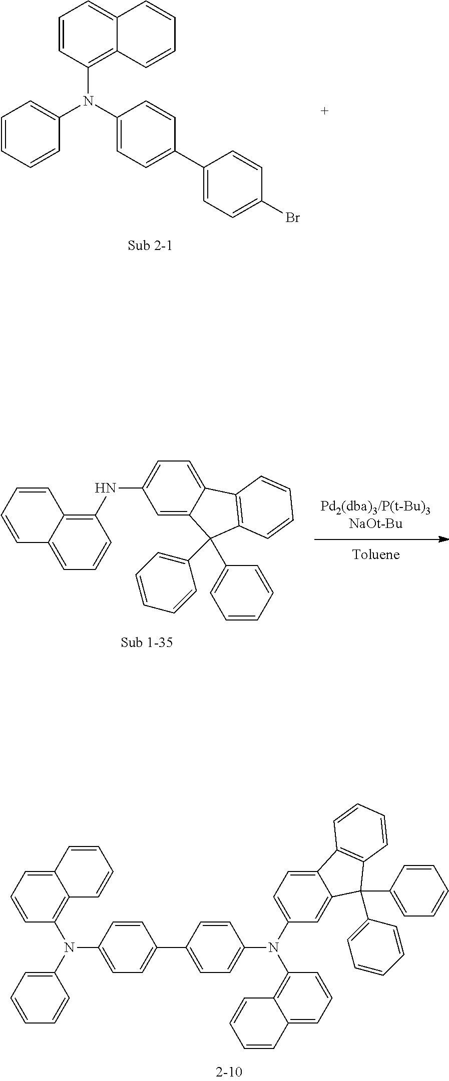 [SMILES: Brc1ccc(-c2ccc(N(c3ccccc3)c3cccc4ccccc34)cc2)cc1 ; c1ccc(C2(c3ccccc3)c3ccccc3-c3ccc(Nc4cccc5ccccc45)cc32)cc1 ; c1ccc(N(c2ccc(-c3ccc(N(c4ccc5c(c4)C(c4ccccc4)(c4ccccc4)c4ccccc4-5)c4cccc5ccccc45)cc3)cc2)c2cccc3ccccc23)cc1]

Sub 2-1 (8 g, 17.76 mmol), toluene (187 ml), Sub 1-35 (8.98 g, 19.54 mmol), Pd₂(dba)₃ (0.49 g, 0.53 mmol), P(t-Bu)₃ (0.36 g, 1.78 mmol), NaOt-Bu (5.12 g, 53.29 mmol) were carried out in the same manner as 2-1 to obtain 10.6 g of the product. (Yield: 72%)

### Synthesis of 2-23

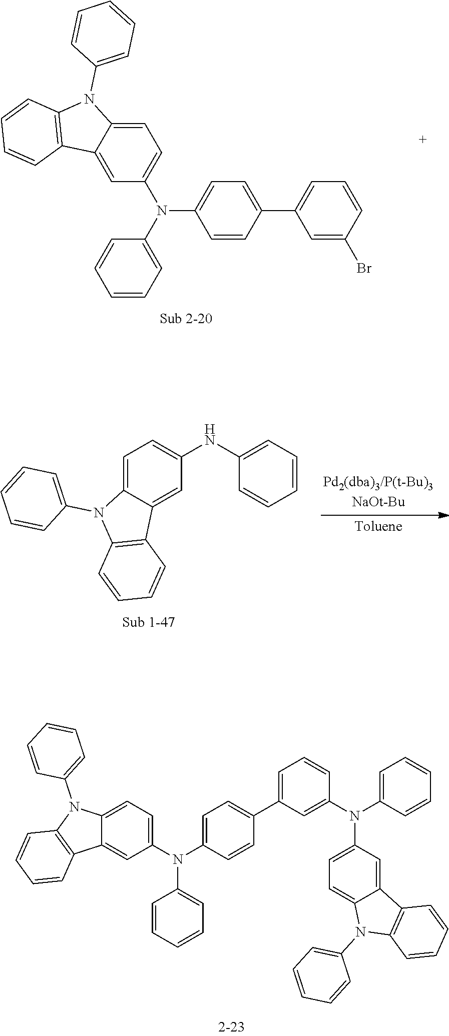 [SMILES: Brc1cccc(-c2ccc(N(c3ccccc3)c3ccc4c(c3)c3ccccc3n4-c3ccccc3)cc2)c1 ; c1ccc(Nc2ccc3c(c2)c2ccccc2n3-c2ccccc2)cc1 ; c1ccc(N(c2ccc(-c3cccc(N(c4ccccc4)c4ccc5c(c4)c4ccccc4n5-c4ccccc4)c3)cc2)c2ccc3c(c2)c2ccccc2n3-c2ccccc2)cc1]

Sub 2-20 (9 g, 15.91 mmol), toluene (167 ml), Sub 1-47 (5.85 g, 17.51 mmol), Pd₂(dba)₃ (0.44 g, 0.48 mmol), P(t-Bu)₃ (0.32 g, 1.59 mmol), NaOt-Bu (4.59 g, 47.74 mmol) were carried out in the same manner as 2-1 to obtain 10.04 g of the product. (Yield: 77%)

### Synthesis of 2-25

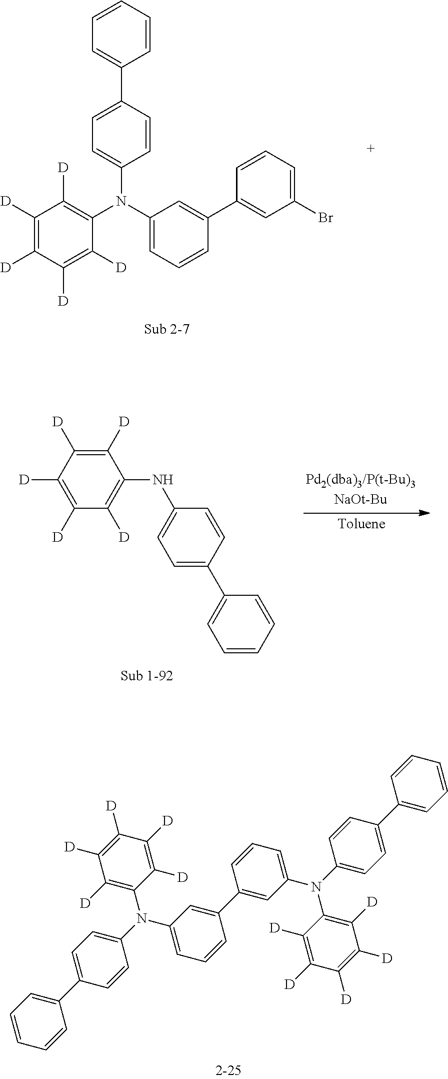 [SMILES: [2H]c1c([2H])c([2H])c(N(c2ccc(-c3ccccc3)cc2)c2cccc(-c3cccc(Br)c3)c2)c([2H])c1[2H] ; [2H]c1c([2H])c([2H])c(Nc2ccc(-c3ccccc3)cc2)c([2H])c1[2H] ; [2H]c1c([2H])c([2H])c(N(c2ccc(-c3ccccc3)cc2)c2cccc(-c3cccc(N(c4ccc(-c5ccccc5)cc4)c4c([2H])c([2H])c([2H])c([2H])c4[2H])c3)c2)c([2H])c1[2H]]

Sub 2-7 (10 g, 20.77 mmol), toluene (218 ml), Sub 1-92 (5.72 g, 22.85 mmol), Pd₂(dba)₃ (0.57 g, 0.62 mmol), P(t-Bu)₃ (0.42 g, 2.08 mmol), NaOt-Bu (5.99 g, 62.31 mmol) were carried out in the same manner as 2-1 to obtain 10.68 g of the product. (Yield: 79%)

### Synthesis of 2-27

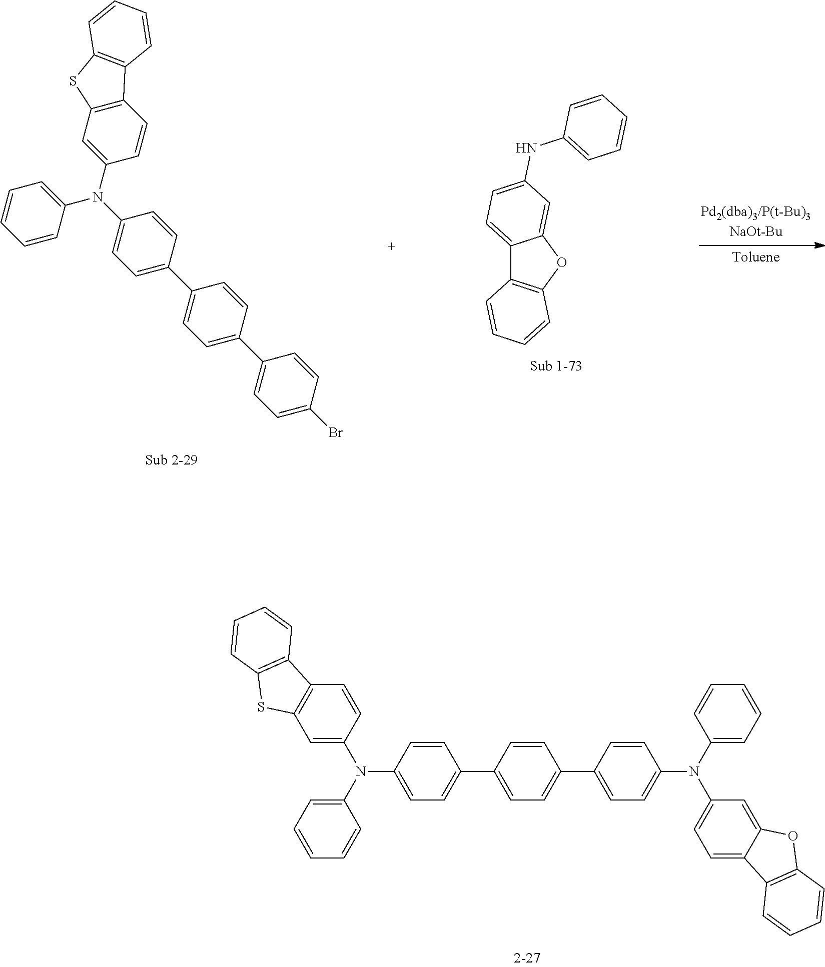 [SMILES: Brc1ccc(-c2ccc(-c3ccc(N(c4ccccc4)c4ccc5c(c4)sc4ccccc45)cc3)cc2)cc1 ; c1ccc(Nc2ccc3c(c2)oc2ccccc23)cc1 ; c1ccc(N(c2ccc(-c3ccc(-c4ccc(N(c5ccccc5)c5ccc6c(c5)sc5ccccc56)cc4)cc3)cc2)c2ccc3c(c2)oc2ccccc23)cc1]

Sub 2-29 (11 g, 18.88 mmol), toluene (198 ml), Sub 1-73 (5.39 g, 20.77 mmol), Pd₂(dba)₃ (0.52 g, 0.57 mmol), P(t-Bu)₃ (0.38 g, 1.89 mmol), NaOt-Bu (5.44 g, 56.65 mmol) were carried out in the same manner as 2-1 to obtain 10.49 g of the product. (Yield: 73%)

### Synthesis of 2-41

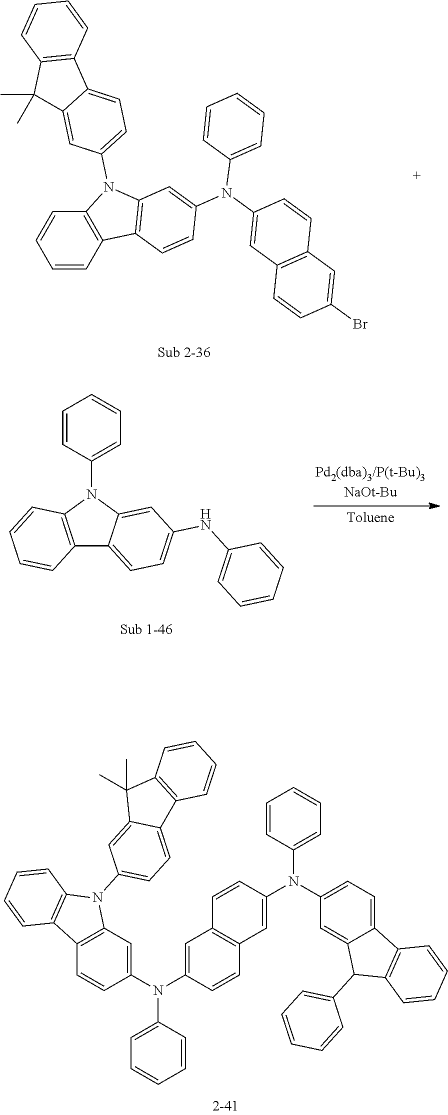 [SMILES: CC1(C)c2ccccc2-c2ccc(-n3c4ccccc4c4ccc(N(c5ccccc5)c5ccc6cc(Br)ccc6c5)cc43)cc21 ; c1ccc(Nc2ccc3c4ccccc4n(-c4ccccc4)c3c2)cc1 ; CC1(C)c2ccccc2-c2ccc(-n3c4ccccc4c4ccc(N(c5ccccc5)c5ccc6cc(N(c7ccccc7)c7ccc8c(c7)C(c7ccccc7)c7ccccc7-8)ccc6c5)cc43)cc21]

Sub 2-36 (11 g, 16.78 mmol), toluene (176 ml), Sub 1-46 (6.17 g, 18.46 mmol), Pd₂(dba)₃ (0.46 g, 0.50 mmol), P(t-Bu)₃ (0.34 g, 1.68 mmol), NaOt-Bu (4.84 g, 50.33 mmol) were carried out in the same manner as 2-1 to obtain 10.22 g of the product. (Yield: 67%)

### Synthesis of 2-50

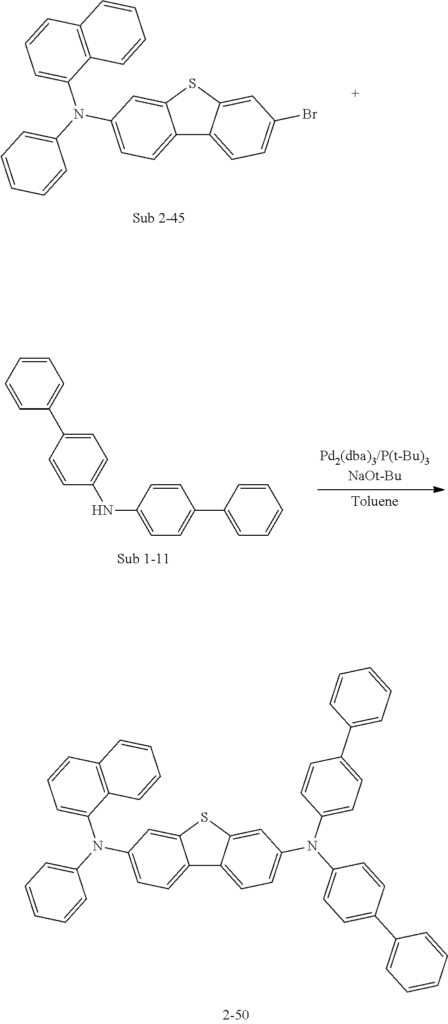 [SMILES: Brc1ccc2c(c1)sc1cc(N(c3ccccc3)c3cccc4ccccc34)ccc12 ; c1ccc(-c2ccc(Nc3ccc(-c4ccccc4)cc3)cc2)cc1 ; c1ccc(-c2ccc(N(c3ccc(-c4ccccc4)cc3)c3ccc4c(c3)sc3cc(N(c5ccccc5)c5cccc6ccccc56)ccc34)cc2)cc1]

Sub 2-45 (9.5 g, 19.77 mmol), toluene (208 ml), Sub 1-11 (6.99 g, 21.75 mmol), Pd₂(dba)₃ (0.54 g, 0.59 mmol), P(t-Bu)₃ (0.40 g, 1.98 mmol), NaOt-Bu (5.70 g, 59.32 mmol) were carried out in the same manner as 2-1 to obtain 10.41 g of the product. (Yield: 73%)

Meanwhile, FD-MS values of the compounds 1-1 to 1-60 and 2-1 to 2-70 of the present invention prepared according to the above synthesis examples are shown in Table 3 below.

TABLE 3

| compound | FD-MS | compound | FD-MS |
|---|---|---|---|
| 1-1 | m/z = 473.21(C₃₆H₂₇N = 473.61) | 1-2 | m/z = 523.23(C₄₀H₂₉N = 523.66) |
| 1-3 | m/z = 573.25(C₄₄H₃₁N = 573.72) | 1-4 | m/z = 623.26(C₄₈H₃₃N = 623.78) |
| 1-5 | m/z = 738.30(C₅₆H₃₈N₂ = 738.91) | 1-6 | m/z = 688.29(C₅₂H₃₆N₂ = 688.87) |
| 1-7 | m/z = 653.28(C₄₈H₃₅N₃ = 653.81) | 1-8 | m/z = 820.36(C₆₀H₄₄N₄ = 821.02) |
| 1-9 | m/z = 727.30(C₅₄H₃₇N₃ = 727.89) | 1-10 | m/z = 668.23(C₄₈H₃₂N₂S = 668.85) |
| 1-11 | m/z = 802.30(C₆₀H₃₈N₂O = 802.98) | 1-12 | m/z = 698.19(C₄₈H₃₀N₂S₋ = 698.90) |
| 1-13 | m/z = 911.33(C₆₆H₄₅N₃S = 912.17) | 1-14 | m/z = 759.23(C₅₃H₃₃N₃OS = 759.93) |
| 1-15 | m/z = 652.29(C₄₉H₃₆N₂ = 652.84) | 1-16 | m/z = 692.28(C₅₁H₃₆N₂O = 692.84) |
| 1-17 | m/z = 794.37(C₆₀H₄₆N₂ = 795.02) | 1-18 | m/z = 903.3 6(C₆₈H₄₅N₃ = 904.10) |
| 1-19 | m/z = 638.27(C₄₈H₃₄N₂ = 638.80) | 1-20 | m/z = 880.29(C₆₅H₄₀N₂S = 881.11) |
| 1-21 | m/z = 777.31(C₅₈H₃₉N₃ = 777.97) | 1-22 | m/z = 718.24(C₅₂H₃₄N₂S = 718.92) |
| 1-23 | m/z = 652.25(C₄₈H₃₂N₂O = 652.78) | 1-24 | m/z = 688.29(C₅₂H₃₆N₂ = 688.87) |
| 1-25 | m/z = 668.23(C₄₈H₃₂N₂S = 668.85) | 1-26 | m/z = 698.19(C₄₈H₃₀N₂S₂ = 698.90) |
| 1-27 | m/z = 612.26(C₄₆H₃₂N₂ = 612.76) | 1-28 | m/z = 769.26(C₅₅H₃₅N₃S = 769.95) |
| 1-29 | m/z = 782.24(C₅₆H₃₄N₂OS = 782.95) | 1-30 | m/z = 943.39(C₇₁H₄₉N₃ = 944.19) |
| 1-31 | m/z = 835.30(C₆₀H₄₁N₃S = 836.07) | 1-32 | m/z = 714.30(C₅₄H₃₈N₂ = 714.89) |
| 1-33 | m/z = 805.31(C₅₉H₃₉N₃O = 805.96) | 1-34 | m/z = 803.33(C₆₀H₄₁N₃ = 803.99) |
| 1-35 | m/z = 768.26(C₅₆H₃₆N₂S = 768.96) | 1-36 | m/z = 767.33(C₅₈H₃₃D₅N₂ = 767.99) |
| 1-37 | m/z = 778.37(C₅₆H₅₀N₂Si = 779.12) | 1-38 | m/z = 610.24(C₄₃H₃₁FN₂O = 610.73) |
| 1-39 | m/z = 749.24(C₅₀H₃₁N₅O₃ = 749.83) | 1-40 | m/z = 769.26(C₅₅H₃₅N₃S = 769.97) |
| 1-41 | m/z = 742.24(C₅₄H₃₄N₂S = 742.93) | 1-42 | m/z = 732.22(C₅₂H₃₂N₂OS = 732.89) |
| 1-43 | m/z = 731.24(C₅₂H₃₃N₃S = 731.92) | 1-44 | m/z = 652.25(C₄₈H₃₂N₂O = 652.78) |
| 1-45 | m/z = 679.30(C₅₀H₃₇N₃ = 679.87) | 1-46 | m/z = 642.30(C₄₈H₃₈N₂ = 642.83) |
| 1-47 | m/z = 830.29(C₆₁H₃₈N₂O₂ = 830.99) | 1-48 | m/z = 907.36(C₆₇H₄₅N₃O = 908.09) |
| 1-49 | m/z = 776.32(C₅₉H₄₀N₂ = 776.96) | 1-50 | m/z = 865.35(C₆₅H₄₃N₃ = 866.06) |
| 1-51 | m/z = 936.35(C₇₂H₄₄N₂ = 937.13) | 1-52 | m/z = 688.29(C₅₂H₃₆N₂ = 688.86) |
| 1-53 | m/z = 794.28(C₅₈H₃₈N₂S = 795.00) | 1-54 | m/z = 884.29(C₆₄H₄₀N₂OS = 885.08) |
| 1-55 | m/z = 738.30(C₅₆H₃₈N₂ = 738.91) | 1-56 | m/z = 792.31(C₅₉H₄₀N₂O = 792.98) |
| 1-57 | m/z = 907.30(C₆₆H₄₁N₃S = 908.14) | 1-58 | m/z = 879.32(C₆₅H₄₁N₃O = 880.04) |
| 1-59 | m/z = 795.37(C₆₀H₃₇D₅N₂ = 796.02) | 1-60 | m/z = 864.35(C₆₆H₄₄N₂ = 865.07) |
| 2-1 | m/z = 588.26(C₄₄H₃₂N₂ = 588.74) | 2-2 | m/z = 816.35(C₆₂H₄₄N₂ = 817.05) |
| 2-3 | m/z = 792.35(C₆₀H₄₄N₂ = 793.03) | 2-4 | m/z = 763.30(C₅₇H₃₇N₃ = 763.94) |
| 2-5 | m/z = 700.37(C₅₂H₂₈D₁₀N₂ = 700.95) | 2-6 | m/z = 730.33(C₅₅H₄₂N₂ = 730.96) |
| 2-7 | m/z = 877.44(C₆₆H₄₃D₇N₂ = 878.18) | 2-8 | m/z = 876.35(C₆₄H₄₈N₂S = 877.16) |
| 2-9 | m/z = 952.48(C₇₂H₆₀N₂ = 953.29) | 2-10 | m/z = 828.35(C₆₃H₄₄N₂ = 829.06) |
| 2-11 | m/z = 863.33(C₆₂H₄₅N₃S = 864.12) | 2-12 | m/z = 981.41(C₇₄H₅₁N₃ = 982.24) |
| 2-13 | m/z = 816.31(C₆₁H₄₀N₂O = 817.00) | 2-14 | m/z = 770.28(C₅₆H₃₈N₂S = 770.99) |
| 2-15 | m/z = 794.29(C₅₈H₃₈N₂O₂ = 794.95) | 2-16 | m/z = 852.26(C₆₀H₄₀N₂S₂ = 853.10) |
| 2-17 | m/z = 912.18(C₆₀H₃₆N₂S₄ = 913.20) | 2-18 | m/z = 852.26(C₆₀H₄₀N₂S₂ = 853.10) |
| 2-19 | m/z = 905.38(C₆₈H₄₇N₃ = 906.15) | 2-20 | m/z = 935.33(C₆₈H₄₅N₃S = 936.19) |
| 2-21 | m/z = 709.26(C₅₀H₃₅N₃S = 709.91) | 2-22 | m/z = 800.23 (C₅₆H₃₆N₂S₂ = 801.04) |
| 2-23 | m/z = 818.34(C₆₀H₄₂N₄ = 819.02) | 2-24 | m/z = 818.34(C₆₀H₄₂N₄ = 819.02) |
| 2-25 | m/z = 650.35(C₄₈H₂₆D₁₀N₂ = 650.89) | 2-26 | m/z = 664.29(C₅₀H₃₆N₂ = 664.85) |
| 2-27 | m/z = 760.25(C₅₄H₃₆N₂OS = 760.96) | 2-28 | m/z = 664.29(C₅₀H₃₆N₂ = 664.85) |
| 2-29 | m/z = 740.32(C₅₆H₄₀N₂ = 740.95) | 2-30 | m/z = 878.37(C₆₇H₄₆N₂ = 879.12) |
| 2-31 | m/z = 911.33(C₆₆H₄₅N₃S = 912.17) | 2-32 | m/z = 664.29(C₅₀H₃₆N₂ = 664.85) |
| 2-33 | m/z = 844.31(C₆₂H₄₀N₂O₂ = 844.99) | 2-34 | m/z = 664.29(C₅₀H₃₆N₂ = 664.85) |
| 2-35 | m/z = 664.29(C₅₀H₃₆N₂ = 664.85) | 2-36 | m/z = 820.31(C₆₀H₄₀N₂O₂ = 820.99) |
| 2-37 | m/z = 640.29(C₄₈H₃₆N₂ = 640.83) | 2-38 | m/z = 881.38(C₆₆H₄₇N₃ = 882.10) |
| 2-39 | m/z = 764.32(C₅₈H₄₀N₂ = 764.97) | 2-40 | m/z = 648.35(C₄₈H₄₄N₂ = 648.89) |
| 2-41 | m/z = 908.39(C₆₇H₄₈N₄ = 909.15) | 2-42 | m/z = 538.24(C₄₀H₃₀N₂ = 538.69) |
| 2-43 | m/z = 588.26(C₄₄H₃₂N₂ = 588.75) | 2-44 | m/z = 588.26(C₄₄H₃₂N₂ = 588.75) |
| 2-45 | m/z = 764.32(C₅₈H₄₀N₂ = 764.97) | 2-46 | m/z = 846.31(C₆₂H₄₂N₂S = 847.09) |
| 2-47 | m/z = 677.28(C₅₀H₃₅N₃ = 677.85 | 2-48 | m/z = 627.27(C₄₆H₃₃N₃ = 627.79) |
| 2-49 | m/z = 814.21(C₅₆H₃₄N₂OS₂ = 815.02) | 2-50 | m/z = 720.26(C₅₂H₃₆N₂S = 720.93) |
| 2-51 | m/z = 748.27(C₅₂H₃₆N₄S = 748.95) | 2-52 | m/z = 744.26(C₅₄H₃₆N₂S = 744.96) |
| 2-53 | m/z = 774.27(C₅₅H₃₈N₂OS = 774.98) | 2-54 | m/z = 731.33(C₅₄H₄₁N₃ = 731.94) |
| 2-55 | m/z = 670.24(C₄₈H₃₄N₂S = 670.87) | 2-56 | m/z = 866.24(C₆₀H₃₈N₂OS₂ = 867.10) |
| 2-57 | m/z = 698.20(C₄₈H₃₀N₂O₂S = 698.84) | 2-58 | m/z = 850.34(C₆₂H₄₆N₂S = 851.12) |
| 2-59 | m/z = 782.30(C₅₆H₃₈N₄ = 782.95) | 2-60 | m/z = 729.31(C₅₄H₃₉N₃ = 729.93) |
| 2-61 | m/z = 936.44(C₇₁H₅₆N₂ = 937.24) | 2-62 | m/z = 744.28(C₅₄H₃₆N₂O₂ = 744.89) |
| 2-63 | m/z = 792.35(C₆₀H₄₄N₂ = 793.03) | 2-64 | m/z = 756.35(C₅₇H₄₄N₄ = 756.99) |
| 2-65 | m/z = 694.24(C₅₀H₃₄N₂S = 694.90) | 2-66 | m/z = 852.26(C₆₀H₄₀N₂S₂ = 853.11) |
| 2-67 | m/z = 922.40(C₆₈H₅₀N₄ = 923.18) | 2-68 | m/z = 631.27(C₄₄H₃₃N₅ = 631.78) |
| 2-69 | m/z = 626.24(C₄₃H₃₁FN₂O₂ = 626.73) | 2-70 | m/z = 723.40(C₅₁H₄₁D₇N₂Si = 724.09) |

### Synthesis Examples 2

### I. Synthesis of Formula (2)

The final product 2 represented by Formula (2) of the present invention is prepared by reacting Sub 3 and Sub 4 as shown in the following Reaction Scheme 6.

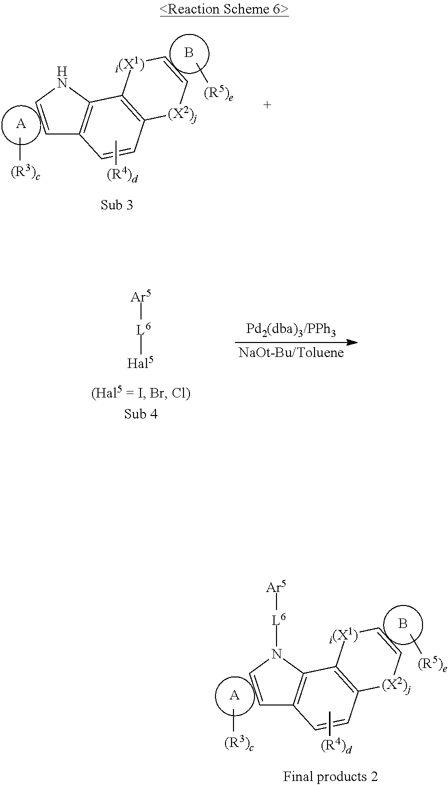

### Synthesis Example of Sub 3

Sub 3 of Reaction Scheme 6 can be synthesized by the reaction path of the following Reaction Scheme 7, but is not limited thereto.

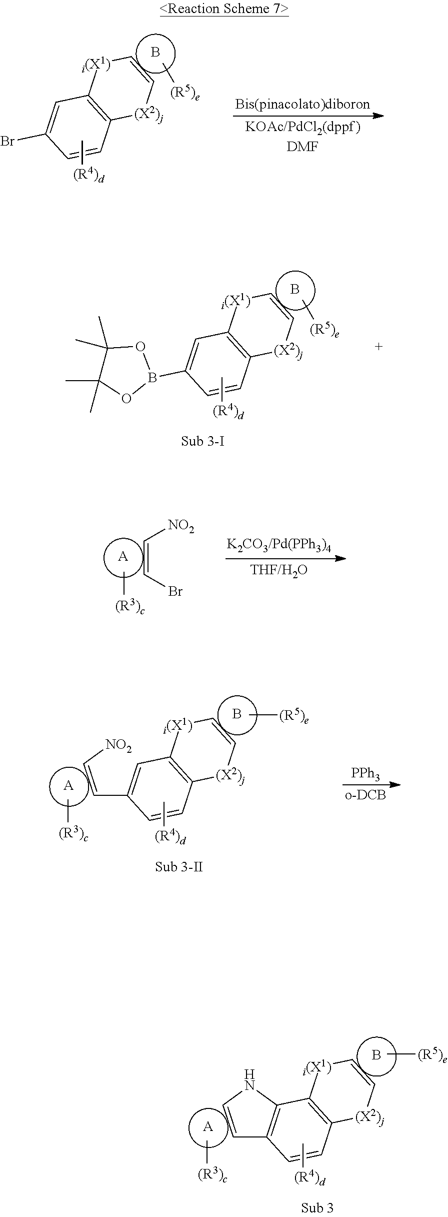

### Synthesis Example of Sub 3-1

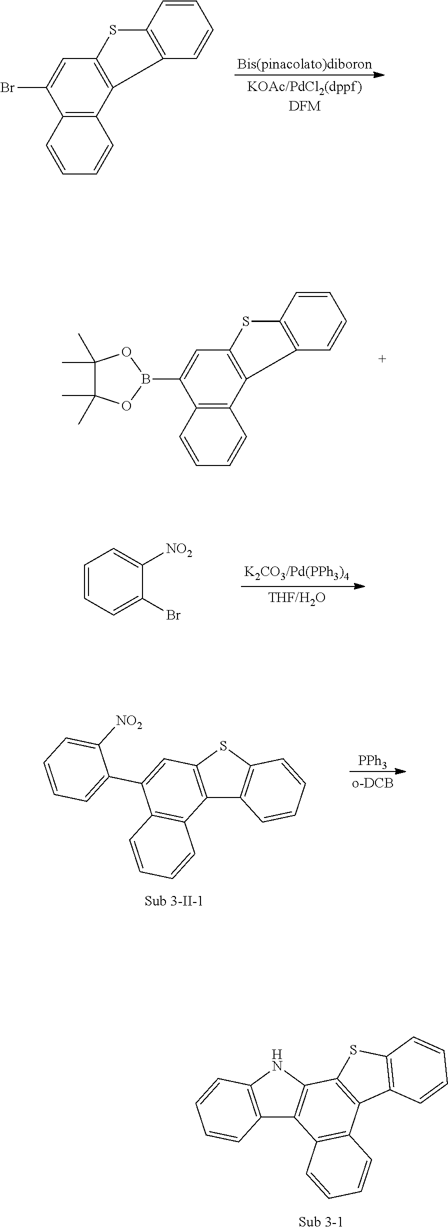 [SMILES: Brc1cc2sc3ccccc3c2c2ccccc12 ; CC1(C)OB(c2cc3sc4ccccc4c3c3ccccc23)OC1(C)C ; O=[N+]([O-])c1ccccc1Br ; O=[N+]([O-])c1ccccc1-c1cc2sc3ccccc3c2c2ccccc12 ; c1ccc2c(c1)[nH]c1c3sc4ccccc4c3c3ccccc3c21 ; "PPh3"]

### (1) Synthesis of Sub 3-I-1

After 5-bromobenzo[b]naphtha[1,2-d]thiophene (50 g, 159.64 mmol), bis(pinacolato)diboron (44.59 g, 175.60 mmol), KOAc (47 g, 478.91 mmol), PdCl₂(dppf) (3.50 g, 4.79 mmol) were dissolved in DMF (1006 mL), and refluxed at 120° C. for 12 hours. When the reaction was completed, the temperature of the reaction was cooled to room temperature, extracted with CH₂Cl₂ and wiped with water. The organic layer was dried over MgSO₄ and concentrated. The resulting compound was recrystallized by CH₂Cl₂ and methanol solvent to obtain the product. (46.01 g, 80%)

### (2) Synthesis of Sub 3-II-1

Sub 3-I-1 (45.94 g, 156.17 mmol), 1-bromo-2-nitrobenzene (38.90 g, 156.17 mmol), K₂CO₃ (64.75 g, 468.51 mmol), Pd(PPh₃)₄ (5.41 g, 4.69 mmol) were added in a round bottom flask and THF (687 mL) and water (344 mL) were added to dissolve and refluxed at 80° C. for 12 hours. When the reaction was completed, the temperature of the reaction was cooled to room temperature, extracted with CH₂Cl₂ and wiped with water. The organic layer was dried over MgSO₄ and concentrated. The resulting compound was separated by silicagel column chromatography to obtain the product. (38.85 g, 70%)

### (3) Synthesis of Sub 3-1

Sub 3-II-1 (38.85 g, 109.31 mmol) and triphenylphosphine (71.68 g, 273.28 mmol) were dissolved in o-dichlorobenzene (547 mL) and refluxed for 24 hours. When the reaction was completed, the solvent was removed using reduced pressure distillation. The resulting compound was separated by silicagel column chromatography and recrystallized to obtain the product. (25.81 g, 73%)

### Synthesis of Sub 3-2

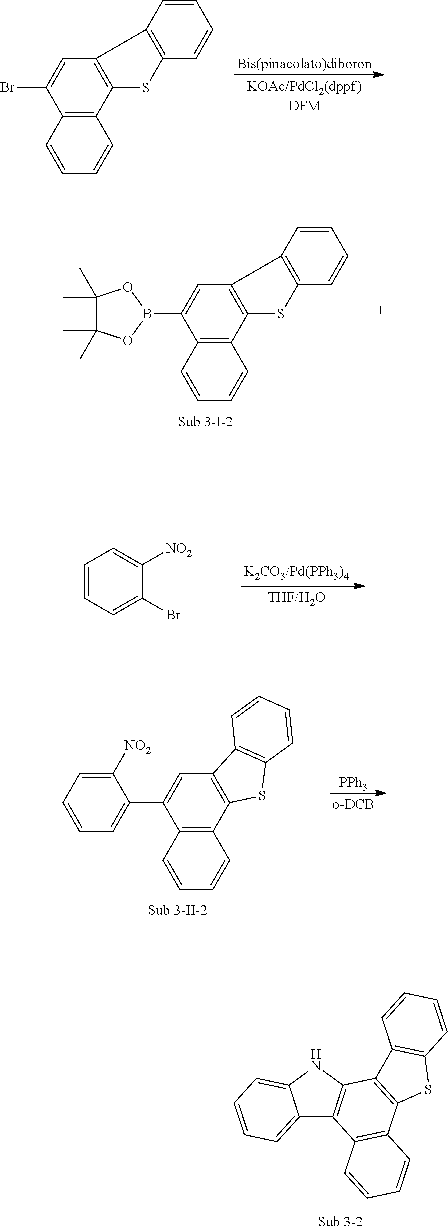 [SMILES: Brc1cc2c3ccccc3sc2c2ccccc12 ; CC1(C)OB(c2cc3c4ccccc4sc3c3ccccc23)OC1(C)C ; O=[N+]([O-])c1ccccc1Br ; O=[N+]([O-])c1ccccc1-c1cc2c3ccccc3sc2c2ccccc12 ; c1ccc2c(c1)[nH]c1c2c2ccccc2c2sc3ccccc3c12 ; "PPh3"]

### (1) Synthesis of Sub 3-I-2

5-bromobenzo[b]naphtho[2,1-d]thiophene (55 g, 175.60 mmol), bis(pinacolato)diboron (49.05 g, 193.16 mmol), KOAc (51.7 g, 526.80 mmol), PdCl₂(dppf) (3.86 g, 5.27 mmol) and DMF (1.11 L) were carried out in the same manner as in Sub 3-I-1 to give the product. (49.35 g, 78%).

### (2) Synthesis of Sub 3-II-2

Sub 3-I-2 (49.22 g, 136.63 mmol), 1-bromo-2-nitrobenzene (27.60 g, 136.63 mmol), K₂CO₃ (56.65 g, 409.88 mmol), Pd(PPh₃)₄ (4.74 g, 4.1 mmol), THF (601 ml) and water (301 ml) were carried out in the same manner as in Sub 3-II-1 to give the product. (26.16 g, 67%).

### (3) Synthesis of Sub 3-2

Sub 3-II-2 (26.16 g, 73.61 mmol), triphenylphosphine (48.26 g, 184.01 mmol), o-dichlorobenzene (368 ml) were carried out in the same manner as in Sub 3-1 to give the product. (15.95 g, 67%).

### Synthesis of Sub 3-7

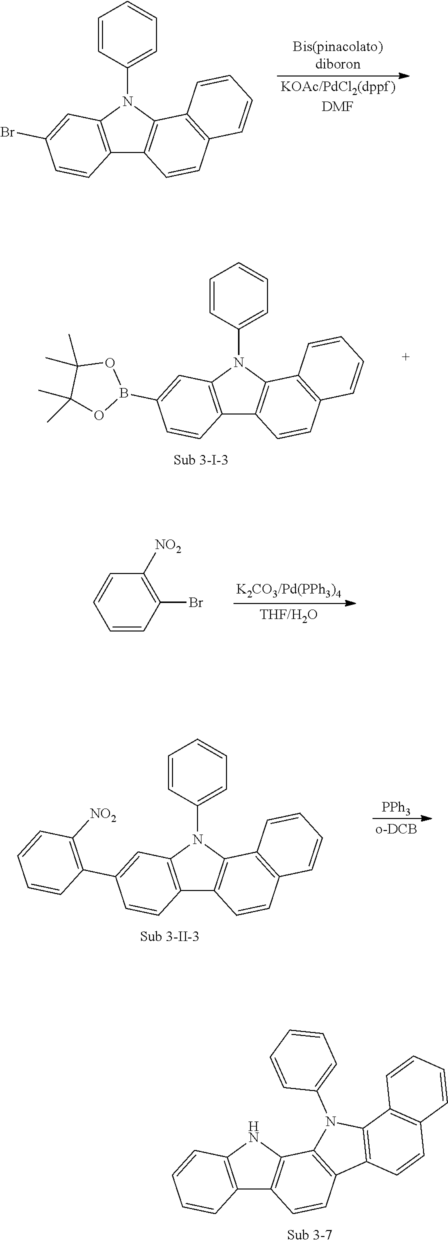 [SMILES: Brc1ccc2c3ccc4ccccc4c3n(-c3ccccc3)c2c1 ; CC1(C)OB(c2ccc3c4ccc5ccccc5c4n(-c4ccccc4)c3c2)OC1(C)C ; O=[N+]([O-])c1ccccc1Br ; O=[N+]([O-])c1ccccc1-c1ccc2c3ccc4ccccc4c3n(-c3ccccc3)c2c1 ; c1ccc(-n2c3c4ccccc4ccc3c3ccc4c5ccccc5[nH]c4c32)cc1 ; "PPh3"]

### (1) Synthesis of Sub 3-I-3

9-bromo-11-phenyl-11H-benzo[a]carbazole (55 g, 147.74 mmol), bis(pinacolato)diboron (41.27 g, 162.52 mmol), KOAc (43.5 g, 443.23 mmol), PdCl₂(dppf) (3.24 g, 4.43 mmol) were carried out in the same manner as in Sub 3-I-1 to give the product. (51.42 g, 83%).

### (2) Synthesis of Sub 3-II-3

Sub 3-I-3 (51.42 g, 122.62 mmol), 1-bromo-2-nitrobenzene (24.77 g, 122.62 mmol), K₂CO₃ (50.84 g, 367.87 mmol), Pd(PPh₃)₄ (4.25 g, 3.68 mmol)), THF (540 ml) and water (270 ml) were carried out in the same manner as in Sub 3-II-1 to give the product. (38.63 g, 76%).

### (3) Synthesis of Sub 3-7

Sub 3-II-3 (38.63 g, 93.21 mmol), triphenylphosphine (61.12 g, 233.01 mmol) and o-dichlorobenzene (466 mL) were carried out in the same manner as in Sub 3-1 to give the product. (14.97 g, 42%).

### Synthesis of Sub 3-13

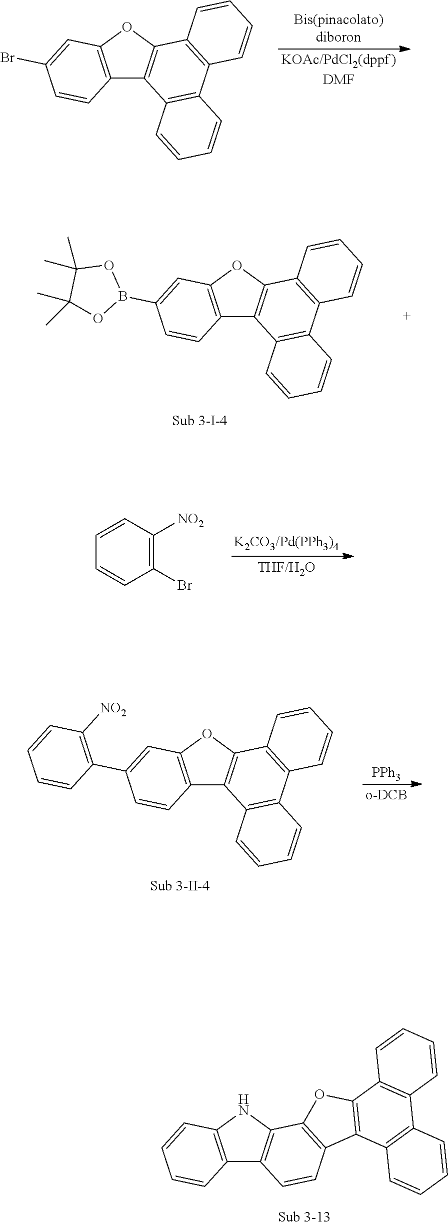 [SMILES: Brc1ccc2c(c1)oc1c3ccccc3c3ccccc3c21 ; CC1(C)OB(c2ccc3c(c2)oc2c4ccccc4c4ccccc4c32)OC1(C)C ; O=[N+]([O-])c1ccccc1Br ; O=[N+]([O-])c1ccccc1-c1ccc2c(c1)oc1c3ccccc3c3ccccc3c21 ; c1ccc2c(c1)[nH]c1c2ccc2c1oc1c3ccccc3c3ccccc3c21 ; "PPh3"]

### (1) Synthesis of Sub 3-I-4

11-bromophenanthro[9,10-b]benzofuran (60 g, 172.81 mmol), bis(pinacolato)diboron (48.27 g, 190.09 mmol), KOAc (50.88 g, 518.42 mmol), PdCl₂(dppf) (3.79 g, 5.18 mmol) were carried out in the same manner as in Sub 3-I-1 to give the product. (52.46 g, 77%).

### (2) Synthesis of Sub 3-II-4

Sub 3-I-4 (52.46 g, 133.05 mmol), 1-bromo-2-nitrobenzene (26.88 g, 133.05 mmol), K₂CO₃ (55.17 g, 399.16 mmol), Pd(PPh₃)₄ (4.61 g, 3.99 mmol), THF (574 ml) and water (287 ml) were carried out in the same manner as in Sub 3-II-1 to give the product. (40.93 g, 79%).

### (3) Synthesis of Sub 3-13

Sub 3-II-4 (40.93 g, 105.11 mmol), triphenylphosphine (68.92 g, 262.77 mmol), o-dichlorobenzene (526 ml) were carried out in the same manner as in Sub 3-1 to give Sub 3-13. (15.03 g, 40%).

### Synthesis of Sub 3-26

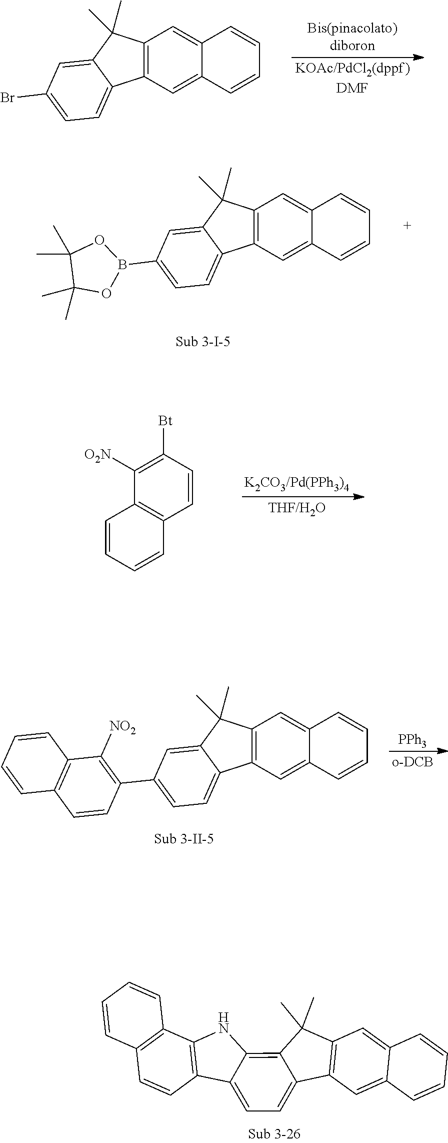

### (1) Synthesis of Sub 3-I-5

2-bromo-11,11-dimethyl-11H-benzo[b]fluorene (60 g, 185.63 mmol), bis(pinacolato)diboron (51.85 g, 204.19 mmol), KOAc (54.65 g, 556.88 mmol), PdCl₂(dppf) (4.08 g, 5.57 mmol) were carried out in the same manner as in Sub 3-I-1 to give the product. (50.87 g, 74%).

### (2) Synthesis of Sub 3-II-5

Sub 3-I-5 (50.87 g, 137.38 mmol), 2-bromo-1-nitronaphthalene (34.63 g, 137.38 mmol), K₂CO₃ (56.96 g, 412.13 mmol), Pd(PPh₃)₄ (4.76 g, 4.12 mmol), THF (568 ml)) and water (284 ml) were carried out in the same manner as in Sub 3-II-1 to give the product. (45.09 g, 79%).

### (3) Synthesis of Sub 3-26

Sub 3-II-5 (45.09 g, 108.52 mmol), triphenylphosphine (71.16 g, 271.31 mmol), o-dichlorobenzene (543 ml) were carried out in the same manner as in Sub 3-1 to give the product. (15.81 g, 38%).

### Synthesis of Sub 3-39

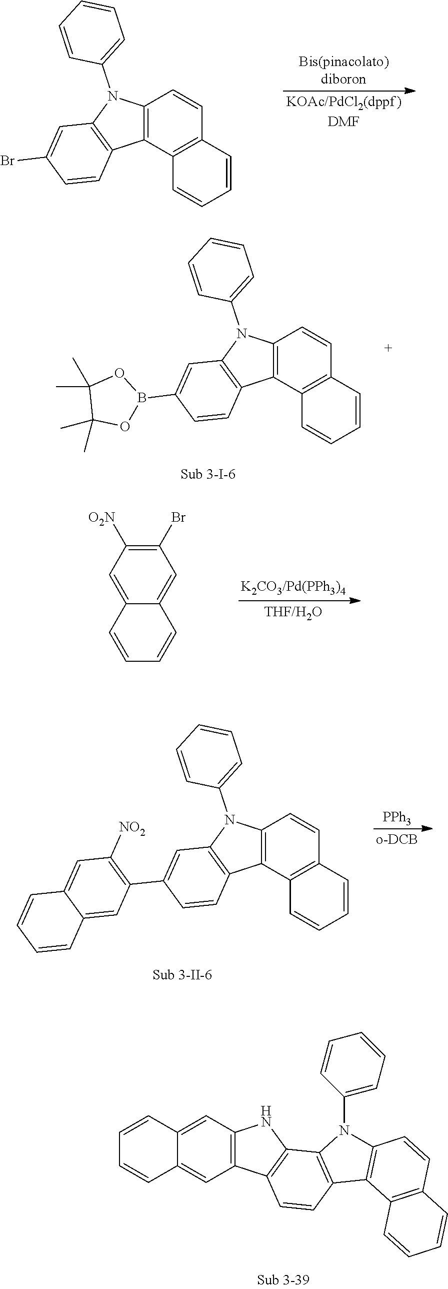

### (1) Synthesis of Sub 3-I-6

9-bromo-7-phenyl-7H-benzo[c]carbazole (60 g, 161.17 mmol), bis(pinacolato)diboron (45.02 g, 177.29 mmol), KOAc (47.45 g, 483.52 mmol), PdCl₂(dppf) (3.54 g, 4.84 mmol) were carried out in the same manner as in Sub 3-I-1 to give the product. (52.04 g, 77%).

### (2) Synthesis of Sub 3-II-6

Sub 3-I-6 (52.04 g, 124.10 mmol), 2-bromo-3-nitronaphthalene (31.28 g, 124.10 mmol), K₂CO₃ (51.46 g, 372.31 mmol), Pd(PPh₃)₄ (4.30 g, 3.72 mmol), THF (546 ml) and water (273 ml) were carried out in the same manner as in Sub 3-II-1 to give the product. (45.54 g, 79%).

### (3) Synthesis of Sub 3-39

Sub 3-II-6 (45.54 g, 98.04 mmol), triphenylphosphine (64.29 g, 245.09 mmol), o-dichlorobenzene (490 ml) were carried out in the same manner as in Sub 3-1 to give the product. (16.96 g, 40%).

### Synthesis of Sub 3-45

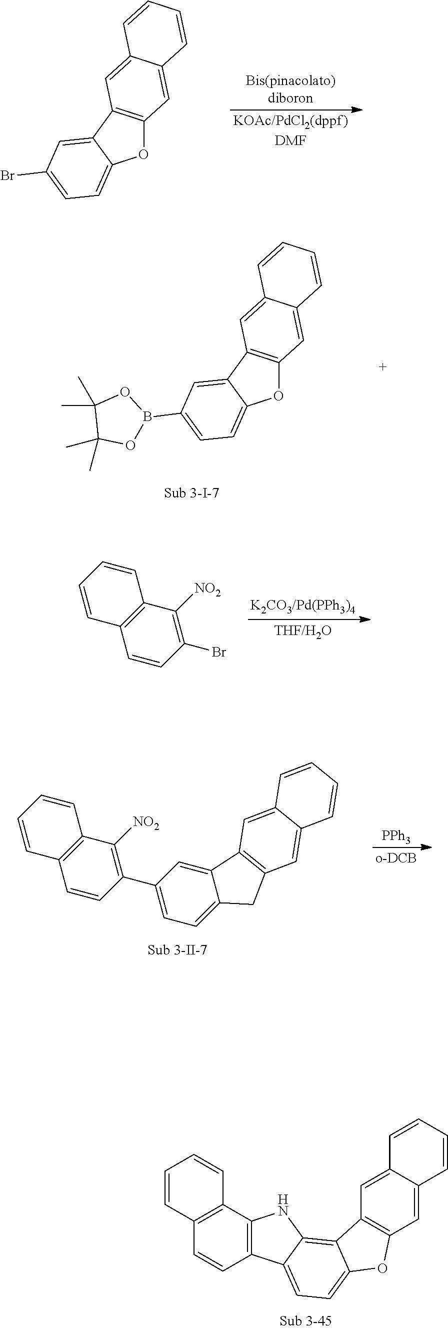 [SMILES: Brc1ccc2oc3cc4ccccc4cc3c2c1 ; CC1(C)OB(c2ccc3oc4cc5ccccc5cc4c3c2)OC1(C)C ; O=[N+]([O-])c1c(Br)ccc2ccccc12 ; O=[N+]([O-])c1c(-c2ccc3c(c2)-c2cc4ccccc4cc2C3)ccc2ccccc12 ; c1ccc2cc3c(cc2c1)oc1ccc2c4ccc5ccccc5c4[nH]c2c13 ; "PPh3"]

### (1) Synthesis of Sub 3-I-7

2-bromonaphtho[2,3-b]benzofuran (60 g, 201.92 mmol), bis(pinacolato)diboron (56.40 g, 222.11 mmol), KOAc (59.45 g, 605.75 mmol), PdCl₂(dppf) (4.43 g, 6.06 mmol) were carried out in the same manner as in Sub 3-I-1 to give the product. (55.60 g, 80%).

### (2) Synthesis of Sub 3-II-7

Sub 3-I-7 (55.60 g, 161.52 mmol), 1-bromo-2-nitronaphthalene (40.72 g, 161.52 mmol), K₂CO₃ (66.97 g, 484.57 mmol), Pd(PPh₃)₄ (5.60 g, 4.85 mmol), THF (711 ml) and water (355 ml) were carried out in the same manner as in Sub 3-II-1 to give the product. (45.55 g, 74%).

### (3) Synthesis of Sub 3-45

Sub 3-II-7 (45.55 g, 116.97 mmol), triphenylphosphine (76.7 g, 292.43 mmol), o-dichlorobenzene (585 ml) were carried out in the same manner as in Sub 3-1 to give the product. (15.89 g, 38%).

### Synthesis of Sub 3-61

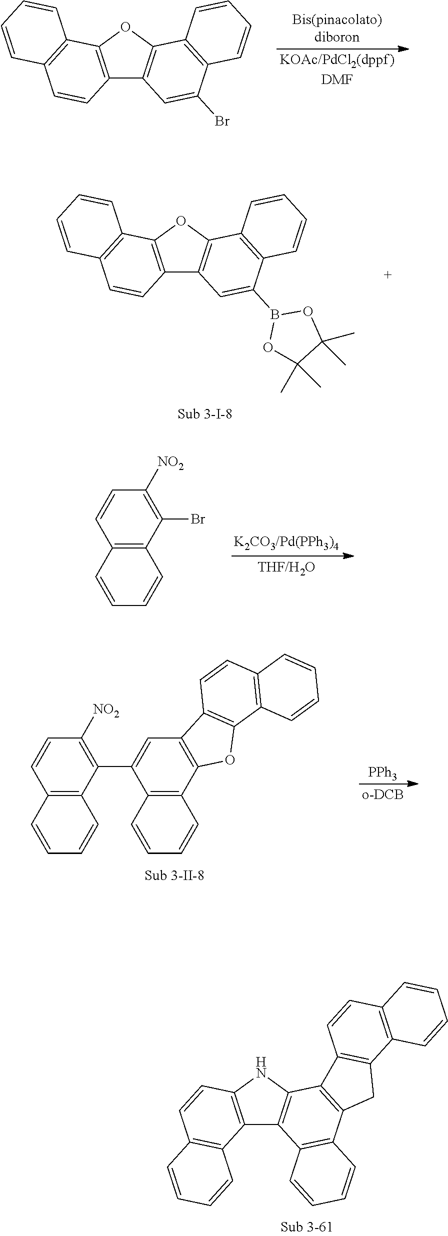 [SMILES: Brc1cc2c3ccc4ccccc4c3oc2c2ccccc12 ; CC1(C)OB(c2cc3c4ccc5ccccc5c4oc3c3ccccc23)OC1(C)C ; O=[N+]([O-])c1ccc2ccccc2c1Br ; O=[N+]([O-])c1ccc2ccccc2c1-c1cc2c3ccc4ccccc4c3oc2c2ccccc12 ; c1ccc2c3c(ccc2c1)-c1c(c2ccccc2c2c1[nH]c1ccc4ccccc4c12)C3 ; "PPh3"]

### (1) Synthesis of Sub 3-I-8

5-bromodinaphtho[1,2-b:2′,1′-d]furan (60 g, 172.81 mmol), bis(pinacolato)diboron (48.27 g, 190.09 mmol), KOAc (50.88 g, 518.42 mmol), PdCl₂(dppf) (3.79 g, 5.18 mmol) were carried out in the same manner as in Sub 3-I-1 to give the product. (52.46 g, 77%).

### (2) Synthesis of Sub 3-II-8

Sub 3-I-8 (52.46 g, 133.05 mmol), 1-bromo-2-nitronaphthalene (33.54 g, 133.05 mmol), K₂CO₃ (55.17 g, 399.16 mmol), Pd(PPh₃)₄ (4.61 g, 3.99 mmol), THF (585 ml) and water (293 ml) were carried out in the same manner as in Sub 3-II-1 to give the product. (40.93 g, 70%).

### (3) Synthesis of Sub 3-61

Sub 3-II-8 (40.93 g, 93.13 mmol), triphenylphosphine (61.07 g, 232.84 mmol), o-dichlorobenzene (466 ml) were carried out in the same manner as in Sub 3-1 to give the product. (23.15 g, 61%).

### Synthesis of Sub 3-66

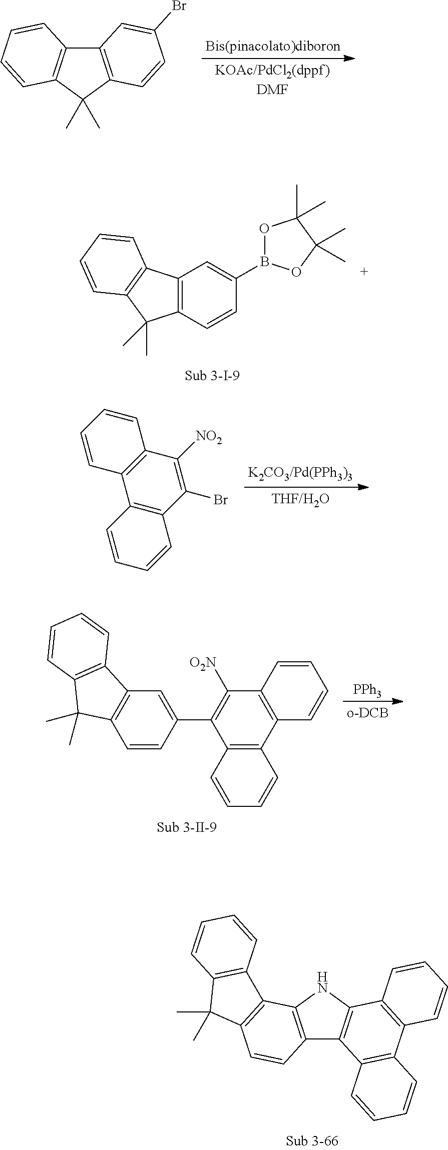 [SMILES: CC1(C)c2ccccc2-c2cc(Br)ccc21 ; CC1(C)c2ccccc2-c2cc(B3OC(C)(C)C(C)(C)O3)ccc21 ; O=[N+]([O-])c1c(Br)c2ccccc2c2ccccc12 ; CC1(C)c2ccccc2-c2cc(-c3c([N+](=O)[O-])c4ccccc4c4ccccc34)ccc21 ; CC1(C)c2ccccc2-c2c1ccc1c2[nH]c2c3ccccc3c3ccccc3c12 ; "PPh3"]

### (1) Synthesis of Sub 3-I-9

Sub 3-I-9 (60 g, 219.64 mmol), bis(pinacolato)diboron (61.35 g, 241.61 mmol), KOAc (64.67 g, 658.93 mmol), PdCl₂(dppf) (4.82 g, 6.59 mmol) were carried out in the same manner as in Sub 3-I-1 to give the product. (52.05 g, 74%).

### (2) Synthesis of Sub 3-II-9

Sub 3-I-8 (52.05 g, 162.53 mmol), 9-bromo-10-nitrophenanthrene (49.11 g, 162.53 mmol), K₂CO₃ (67.39 g, 487.60 mmol), Pd(PPh₃)₄ (5.63 g, 4.88 mmol), THF (715 ml) and water (358 ml) were carried out in the same manner as in Sub 3-II-1 to give the product. (47.95 g, 71%).

### (3) Synthesis of Sub 3-66

Sub 3-II-9 (47.95 g, 115.41 mmol), triphenylphosphine (75.67 g, 288.51 mmol), o-dichlorobenzene (577 ml) were carried out in the same manner as in Sub 3-1 to give the product. (18.59 g, 42%).

### Synthesis of Sub 3-67

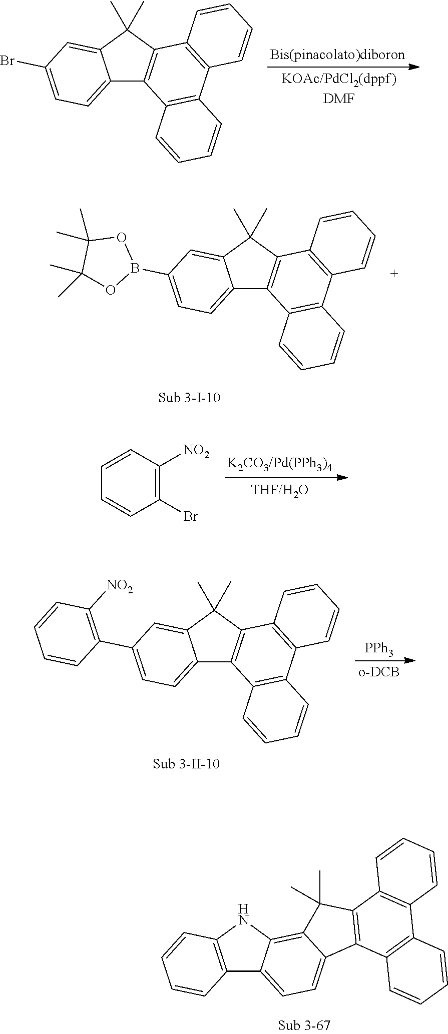 [SMILES: CC1(C)c2cc(Br)ccc2-c2c1c1ccccc1c1ccccc21 ; CC1(C)c2cc(B3OC(C)(C)C(C)(C)O3)ccc2-c2c1c1ccccc1c1ccccc21 ; O=[N+]([O-])c1ccccc1Br ; CC1(C)c2cc(-c3ccccc3[N+](=O)[O-])ccc2-c2c1c1ccccc1c1ccccc21 ; CC1(C)c2c(c3ccccc3c3ccccc23)-c2ccc3c([nH]c4ccccc43)c21 ; "PPh3"]

### (1) Synthesis of Sub 3-I-10

Sub 3-I-10 (60 g, 219.64 mmol), bis(pinacolato)diboron (61.35 g, 241.61 mmol), KOAc (64.67 g, 658.93 mmol), PdCl₂(dppf) (4.82 g, 6.59 mmol) were carried out in the same manner as in Sub 3-I-1 to give the product. (52.05 g, 74%).

### (2) Synthesis of Sub 3-II-10

Sub 3-I-10 (52.05 g, 162.53 mmol), 1-bromo-2-nitrobenzene (49.11 g, 162.53 mmol), K₂CO₃ (67.39 g, 487.60 mmol), Pd(PPh₃)₄ (5.63 g, 4.88 mmol), THF (715 ml) and water (358 ml) were carried out in the same manner as in Sub 3-II-1 to give the product. (47.95 g, 71%).

### (3) Synthesis of Sub 3-67

Sub 3-II-10 (47.95 g, 115.41 mmol), triphenylphosphine (75.67 g, 288.51 mmol), o-dichlorobenzene (577 ml) were carried out in the same manner as in Sub 3-1 to give the product. (18.59 g, 42%).

### Synthesis of Sub 3-79

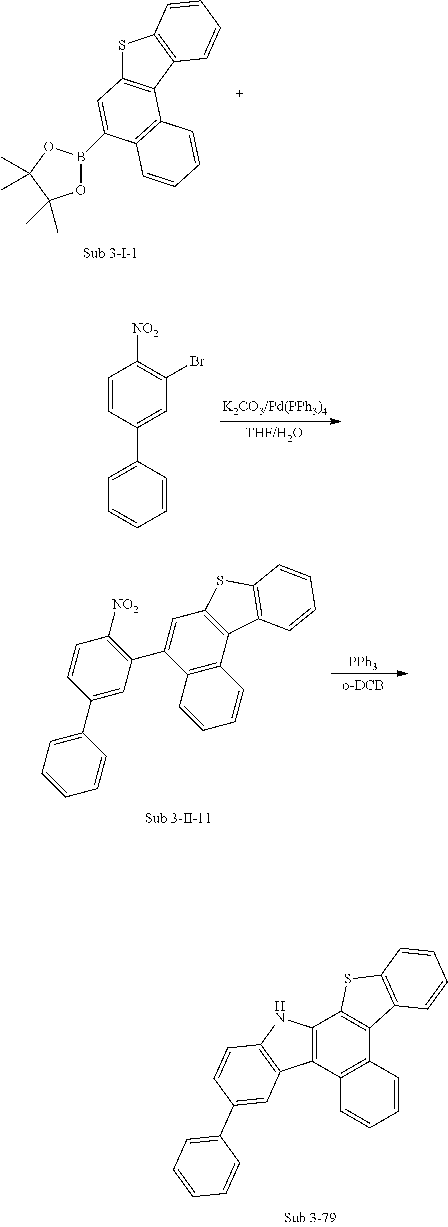 [SMILES: CC1(C)OB(c2cc3sc4ccccc4c3c3ccccc23)OC1(C)C ; O=[N+]([O-])c1ccc(-c2ccccc2)cc1Br ; O=[N+]([O-])c1ccc(-c2ccccc2)cc1-c1cc2sc3ccccc3c2c2ccccc12 ; c1ccc(-c2ccc3[nH]c4c5sc6ccccc6c5c5ccccc5c4c3c2)cc1 ; "PPh3"]

### (1) Synthesis of Sub 3-II-11

Sub 3-I-1 (40 g, 111.02 mmol), 3-bromo-4-nitro-1,1′-biphenyl (30.88 g, 111.02 mmol), K₂CO₃ (46.03 g, 333.07 mmol), Pd(PPh₃)₄ (3.85 g, 3.33 mmol), THF (489 ml) and water (244 ml) were carried out in the same manner as in Sub 3-II-1 to give the product. (34.97 g, 73%).

### (2) Synthesis of Sub 3-79

Sub 3-II-11 (34.97 g, 81.04 mmol), triphenylphosphine (53.14 g, 202.60 mmol), o-dichlorobenzene (405 ml) were carried out in the same manner as in Sub 3-1 to give the product. (21.69 g, 67%).

### Synthesis of Sub 3-82

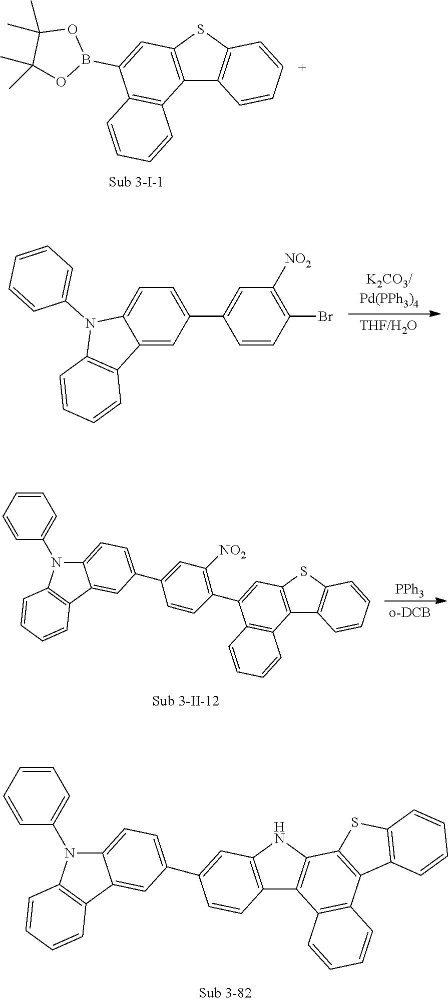 [SMILES: CC1(C)OB(c2cc3sc4ccccc4c3c3ccccc23)OC1(C)C ; O=[N+]([O-])c1cc(-c2ccc3c(c2)c2ccccc2n3-c2ccccc2)ccc1Br ; O=[N+]([O-])c1cc(-c2ccc3c(c2)c2ccccc2n3-c2ccccc2)ccc1-c1cc2sc3ccccc3c2c2ccccc12 ; c1ccc(-n2c3ccccc3c3cc(-c4ccc5c(c4)[nH]c4c6sc7ccccc7c6c6ccccc6c54)ccc32)cc1 ; "PPh3"]

### (1) Synthesis of Sub 3-II-12

Sub 3-I-1 (40 g, 111.02 mmol), 3-(4-bromo-3-nitrophenyl)-9-phenyl-9H-carbazole (49.22 g, 111.02 mmol), K₂CO₃ (46.03 g, 333.07 mmol), Pd(PPh₃)₄ (3.85 g, 3.33 mmol), THF (489 ml) and water (244 ml) were carried out in the same manner as in Sub 3-II-1 to give the product. (42.40 g, 64%).

### (2) Synthesis of Sub 3-82

Sub 3-II-12 (42.40 g, 71.06 mmol), triphenylphosphine (46.59 g, 177.64 mmol), o-dichlorobenzene (355 ml) were carried out in the same manner as in Sub 3-1 to give the product. (24.48 g, 61%).

### Synthesis of Sub 3-88

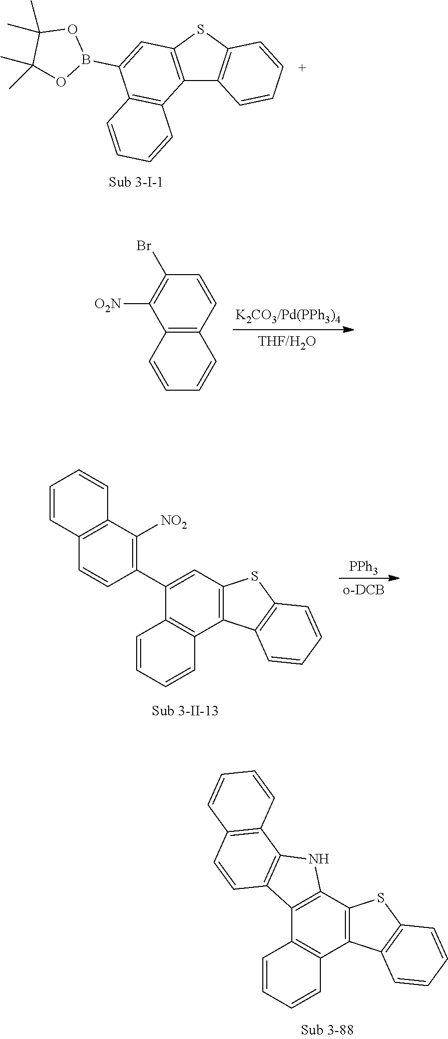 [SMILES: CC1(C)OB(c2cc3sc4ccccc4c3c3ccccc23)OC1(C)C ; O=[N+]([O-])c1c(Br)ccc2ccccc12 ; O=[N+]([O-])c1c(-c2cc3sc4ccccc4c3c3ccccc23)ccc2ccccc12 ; c1ccc2c(c1)ccc1c2[nH]c2c3sc4ccccc4c3c3ccccc3c12 ; "PPh3"]

### (1) Synthesis of Sub 3-II-13

Sub 3-I-1 (57.17 g, 158.69 mmol), 2-bromo-1-nitronaphthalene (40 g, 158.69 mmol), K₂CO₃ (65.80 g, 476.06 mmol), Pd(PPh₃)₄ (5.50 g, 4.76 mmol), THF (698 ml) and water (349 ml) were carried out in the same manner as in Sub 3-II-1 to give the product. (47.61 g, 74%).

### (2) Synthesis of Sub 3-88

Sub 3-II-13 (47.61 g, 117.42 mmol), triphenylphosphine (76.99 g, 293.55 mmol), o-dichlorobenzene (587 ml) were carried out in the same manner as in Sub 3-1 to give the product. (28.07 g, 64%).

Examples of Sub 3 include, but are not limited to, the followings.

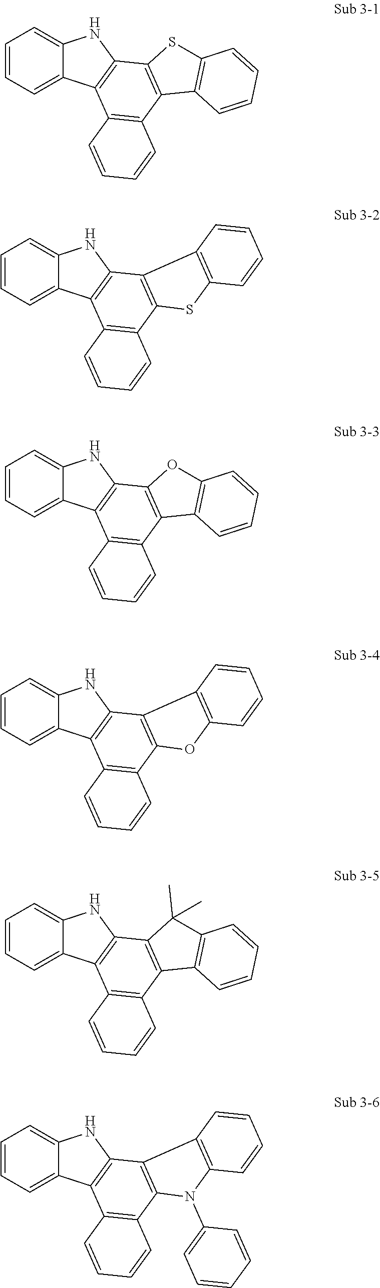 [SMILES: c1ccc2c(c1)[nH]c1c3sc4ccccc4c3c3ccccc3c21 ; c1ccc2c(c1)[nH]c1c2c2ccccc2c2sc3ccccc3c12 ; c1ccc2c(c1)[nH]c1c3oc4ccccc4c3c3ccccc3c21 ; c1ccc2c(c1)[nH]c1c2c2ccccc2c2oc3ccccc3c12 ; CC1(C)c2ccccc2-c2c1c1[nH]c3ccccc3c1c1ccccc21 ; c1ccc(-n2c3ccccc3c3c4[nH]c5ccccc5c4c4ccccc4c32)cc1]

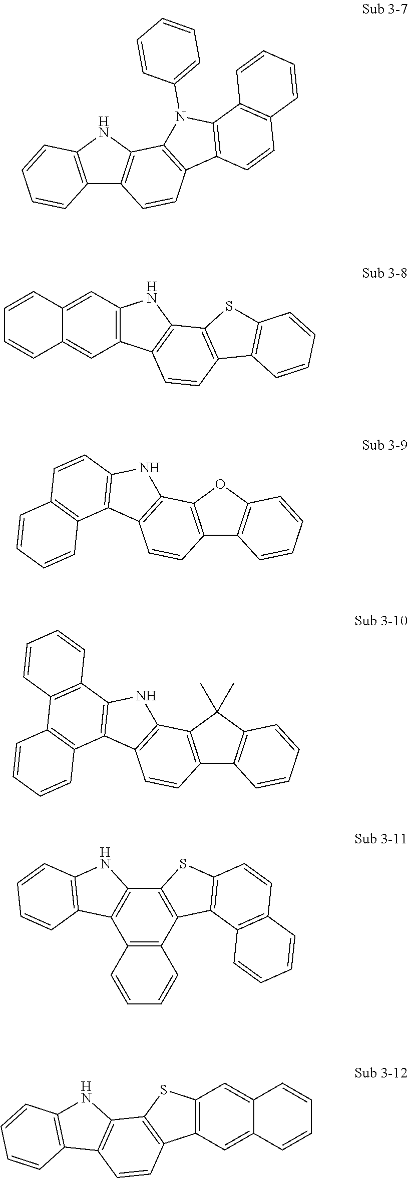 [SMILES: c1ccc(-n2c3c4ccccc4ccc3c3ccc4c5ccccc5[nH]c4c32)cc1 ; c1ccc2cc3c(cc2c1)[nH]c1c3ccc2c3ccccc3sc21 ; c1ccc2c(c1)ccc1[nH]c3c(ccc4c5ccccc5oc43)c12 ; CC1(C)c2ccccc2-c2ccc3c([nH]c4c5ccccc5c5ccccc5c34)c21 ; c1ccc2c(c1)ccc1sc3c4[nH]c5ccccc5c4c4ccccc4c3c12 ; c1ccc2cc3c(cc2c1)sc1c3ccc2c3ccccc3[nH]c21]

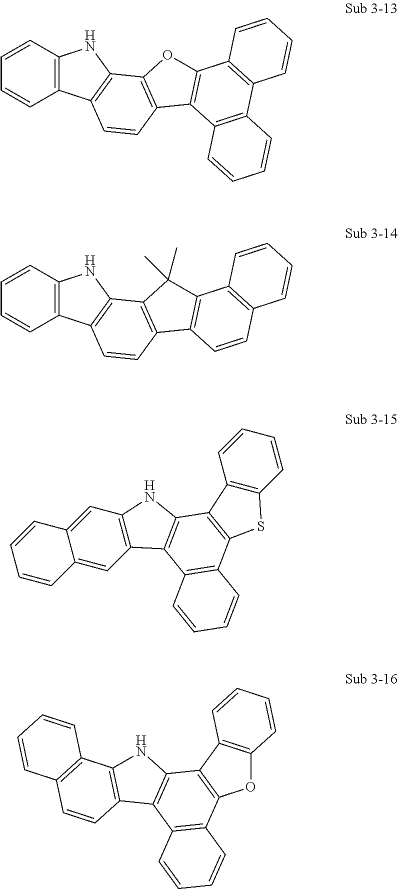 [SMILES: c1ccc2c(c1)[nH]c1c2ccc2c1oc1c3ccccc3c3ccccc3c21 ; CC1(C)c2c(ccc3ccccc23)-c2ccc3c([nH]c4ccccc43)c21 ; c1ccc2cc3c(cc2c1)[nH]c1c3c2ccccc2c2sc3ccccc3c12 ; c1ccc2c(c1)ccc1c2[nH]c2c1c1ccccc1c1oc3ccccc3c21]

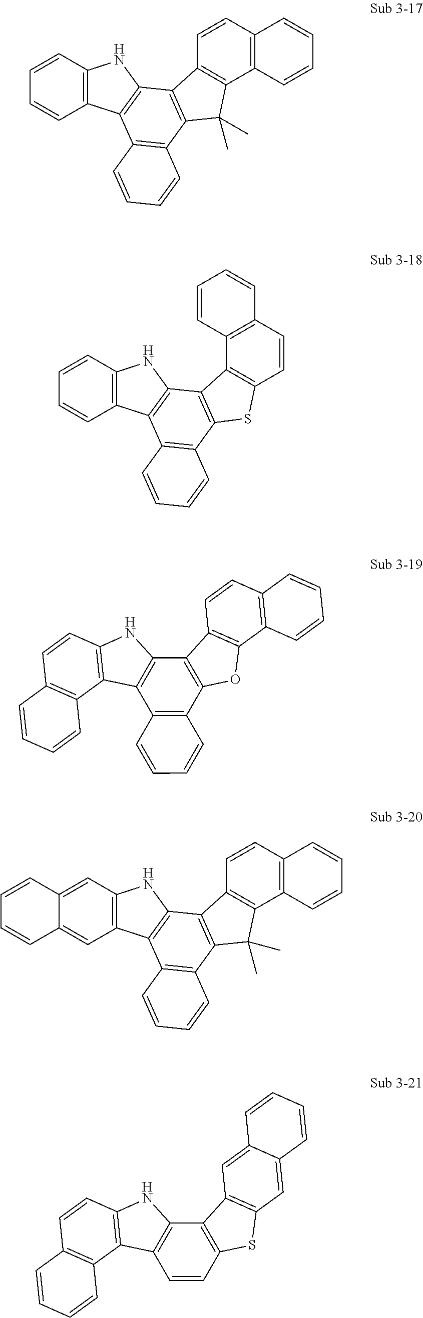 [SMILES: CC1(C)c2c(ccc3ccccc23)-c2c1c1ccccc1c1c2[nH]c2ccccc21 ; c1ccc2c(c1)ccc1sc3c4ccccc4c4c5ccccc5[nH]c4c3c12 ; c1ccc2c(c1)ccc1c2oc2c3ccccc3c3c([nH]c4ccc5ccccc5c43)c12 ; CC1(C)c2c(ccc3ccccc23)-c2c1c1ccccc1c1c2[nH]c2cc3ccccc3cc21 ; c1ccc2cc3c(cc2c1)sc1ccc2c([nH]c4ccc5ccccc5c42)c13]

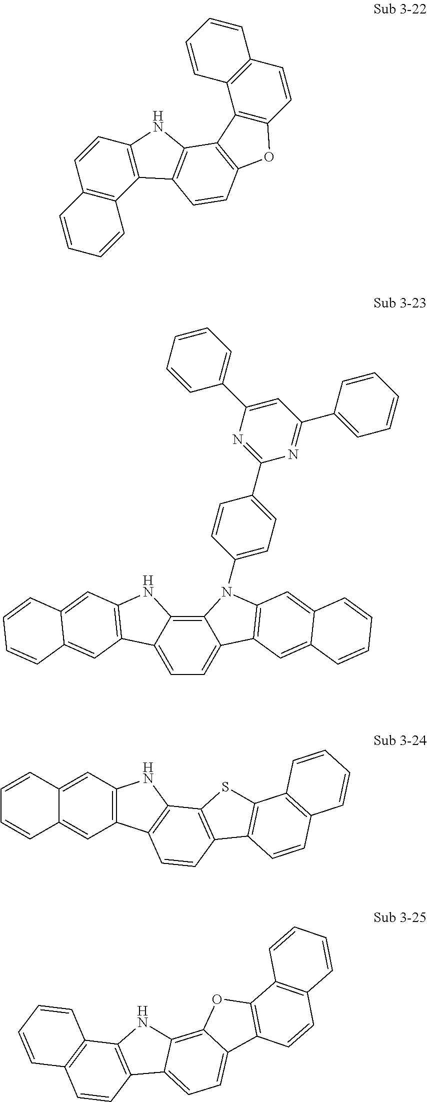 [SMILES: c1ccc2c(c1)ccc1[nH]c3c(ccc4oc5ccc6ccccc6c5c43)c12 ; c1ccc(-c2cc(-c3ccccc3)nc(-c3ccc(-n4c5cc6ccccc6cc5c5ccc6c7cc8ccccc8cc7[nH]c6c54)cc3)n2)cc1 ; c1ccc2cc3c(cc2c1)[nH]c1c3ccc2c3ccc4ccccc4c3sc21 ; c1ccc2c(c1)ccc1c3ccc4c5ccc6ccccc6c5oc4c3[nH]c21]

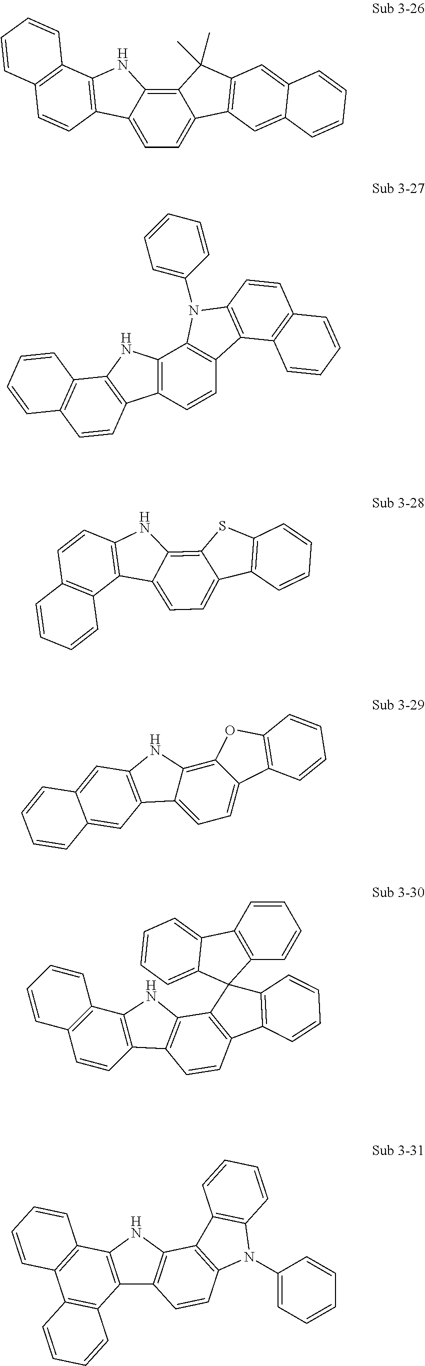 [SMILES: CC1(C)c2cc3ccccc3cc2-c2ccc3c([nH]c4c5ccccc5ccc34)c21 ; c1ccc(-n2c3ccc4ccccc4c3c3ccc4c5ccc6ccccc6c5[nH]c4c32)cc1 ; c1ccc2c(c1)ccc1[nH]c3c(ccc4c5ccccc5sc43)c12 ; c1ccc2cc3c(cc2c1)[nH]c1c3ccc2c3ccccc3oc21 ; c1ccc2c(c1)-c1ccccc1C21c2ccccc2-c2ccc3c([nH]c4c5ccccc5ccc34)c21 ; c1ccc(-n2c3ccccc3c3c4[nH]c5c6ccccc6c6ccccc6c5c4ccc32)cc1]

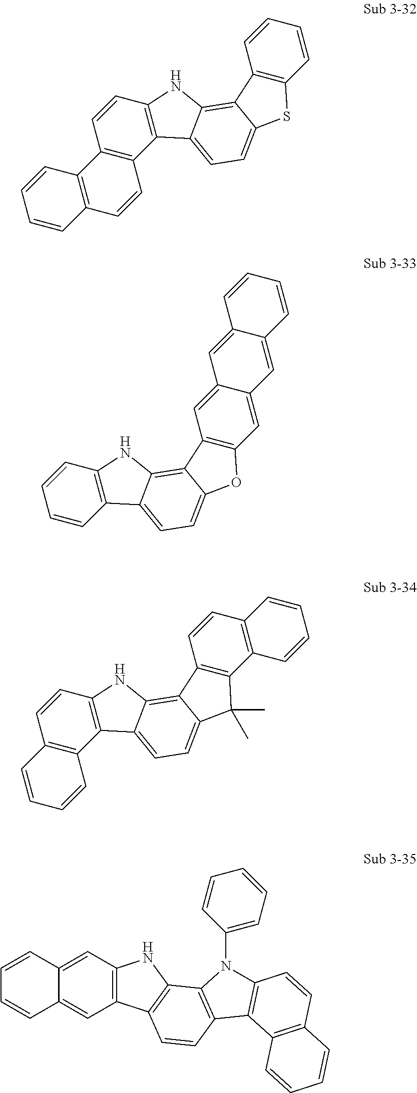 [SMILES: c1ccc2c(c1)ccc1c2ccc2[nH]c3c(ccc4sc5ccccc5c43)c21 ; c1ccc2cc3cc4c(cc3cc2c1)oc1ccc2c3ccccc3[nH]c2c14 ; CC1(C)c2ccc3c([nH]c4ccc5ccccc5c43)c2-c2ccc3ccccc3c21 ; c1ccc(-n2c3ccc4ccccc4c3c3ccc4c5cc6ccccc6cc5[nH]c4c32)cc1]

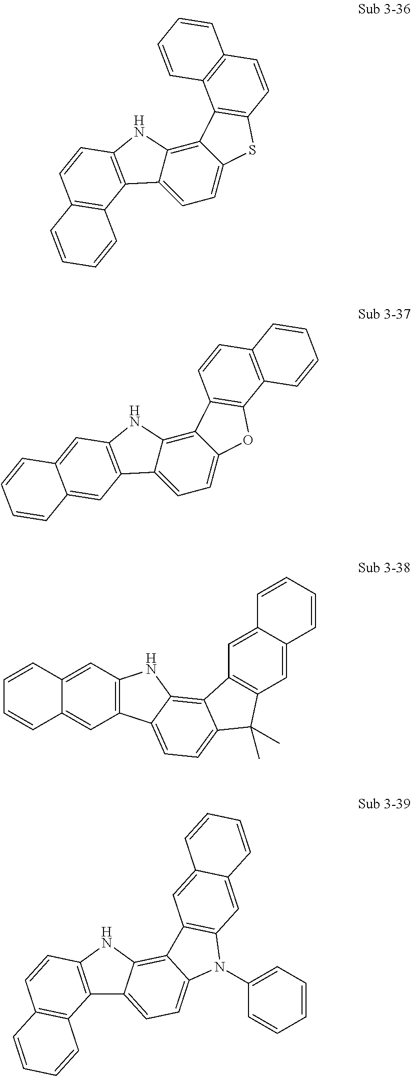 [SMILES: c1ccc2c(c1)ccc1[nH]c3c(ccc4sc5ccc6ccccc6c5c43)c12 ; c1ccc2cc3c(cc2c1)[nH]c1c3ccc2oc3c4ccccc4ccc3c21 ; CC1(C)c2cc3ccccc3cc2-c2c1ccc1c2[nH]c2cc3ccccc3cc21 ; c1ccc(-n2c3cc4ccccc4cc3c3c4[nH]c5ccc6ccccc6c5c4ccc32)cc1]

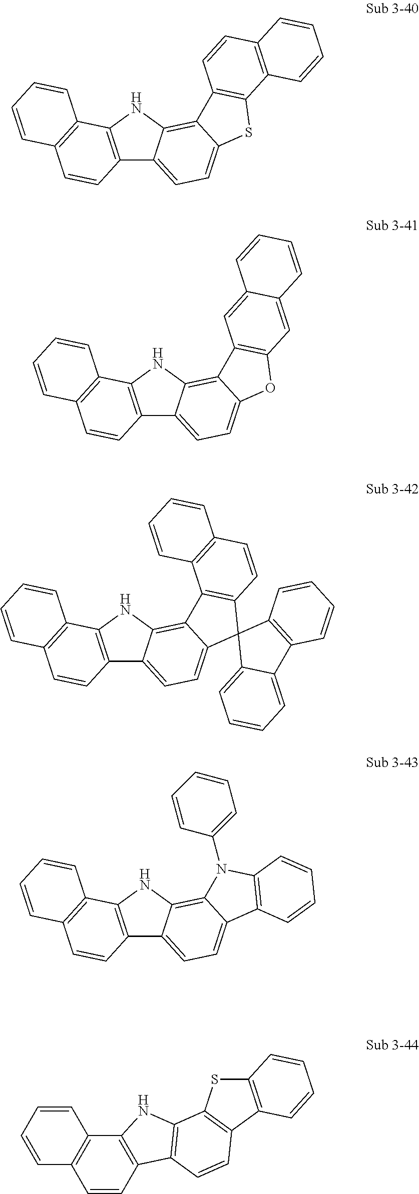 [SMILES: c1ccc2c(c1)ccc1c3ccc4sc5c6ccccc6ccc5c4c3[nH]c21 ; c1ccc2cc3c(cc2c1)oc1ccc2c4ccc5ccccc5c4[nH]c2c13 ; c1ccc2c(c1)-c1ccccc1C21c2ccc3ccccc3c2-c2c1ccc1c2[nH]c2c3ccccc3ccc12 ; c1ccc(-n2c3ccccc3c3ccc4c5ccc6ccccc6c5[nH]c4c32)cc1 ; c1ccc2c(c1)ccc1c3ccc4c5ccccc5sc4c3[nH]c21]

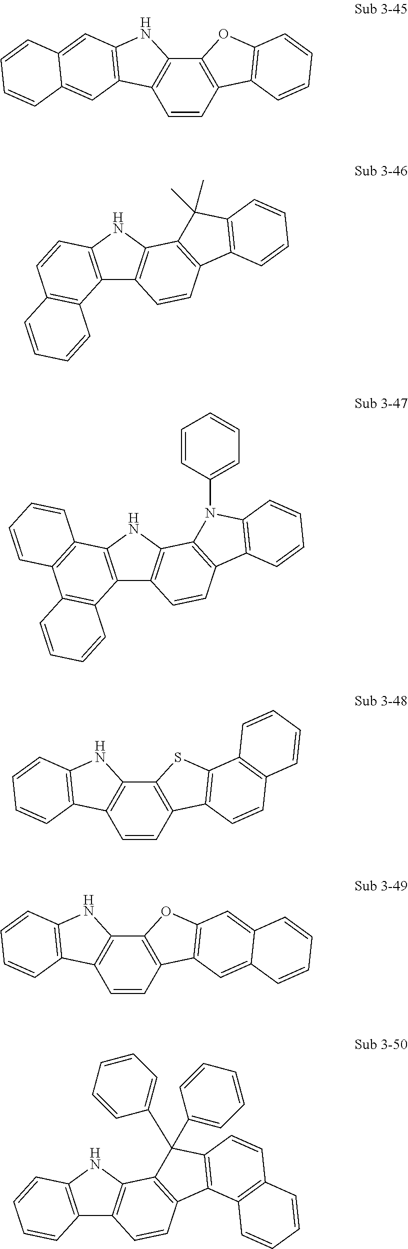 [SMILES: c1ccc2cc3c(cc2c1)[nH]c1c3ccc2c3ccccc3oc21 ; CC1(C)c2ccccc2-c2ccc3c([nH]c4ccc5ccccc5c43)c21 ; c1ccc(-n2c3ccccc3c3ccc4c([nH]c5c6ccccc6c6ccccc6c45)c32)cc1 ; c1ccc2c(c1)ccc1c3ccc4c5ccccc5[nH]c4c3sc21 ; c1ccc2cc3c(cc2c1)oc1c3ccc2c3ccccc3[nH]c21 ; c1ccc(C2(c3ccccc3)c3ccc4ccccc4c3-c3ccc4c([nH]c5ccccc54)c32)cc1]

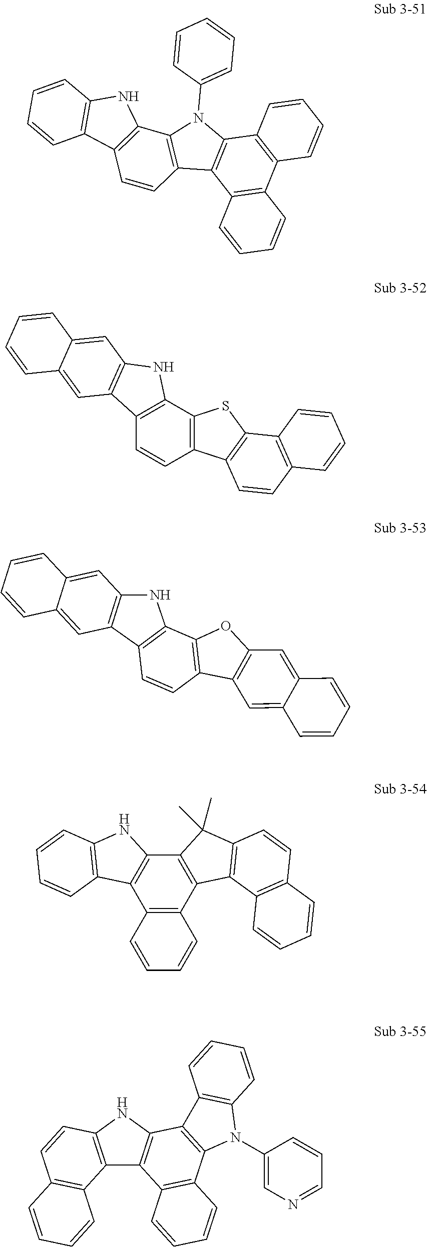 [SMILES: c1ccc(-n2c3c(ccc4c5ccccc5[nH]c43)c3c4ccccc4c4ccccc4c32)cc1 ; c1ccc2cc3c(cc2c1)[nH]c1c3ccc2c3ccc4ccccc4c3sc21 ; c1ccc2cc3c(cc2c1)[nH]c1c3ccc2c3cc4ccccc4cc3oc21 ; CC1(C)c2ccc3ccccc3c2-c2c1c1[nH]c3ccccc3c1c1ccccc21 ; c1cncc(-n2c3ccccc3c3c4[nH]c5ccc6ccccc6c5c4c4ccccc4c32)c1]

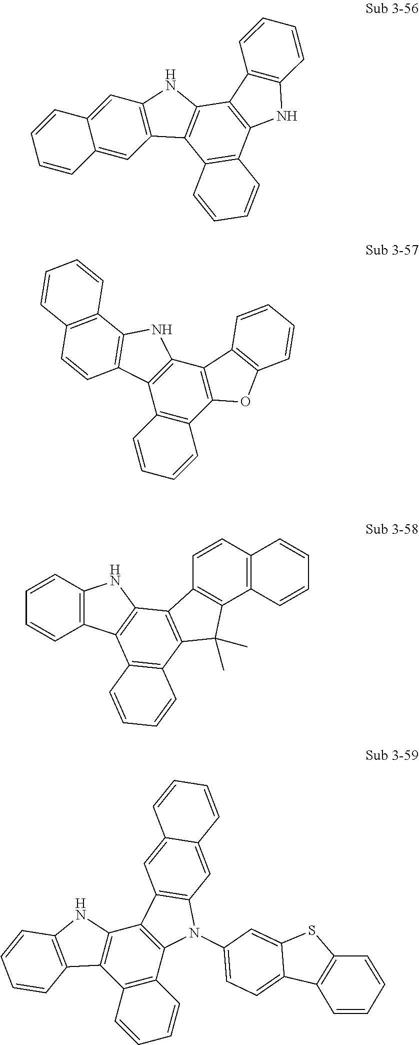 [SMILES: c1ccc2cc3c(cc2c1)[nH]c1c3c2ccccc2c2[nH]c3ccccc3c21 ; c1ccc2c(c1)ccc1c2[nH]c2c1c1ccccc1c1oc3ccccc3c21 ; CC1(C)c2c(ccc3ccccc23)-c2c1c1ccccc1c1c2[nH]c2ccccc21 ; c1ccc2cc3c(cc2c1)c1c2[nH]c4ccccc4c2c2ccccc2c1n3-c1ccc2c(c1)sc1ccccc12]

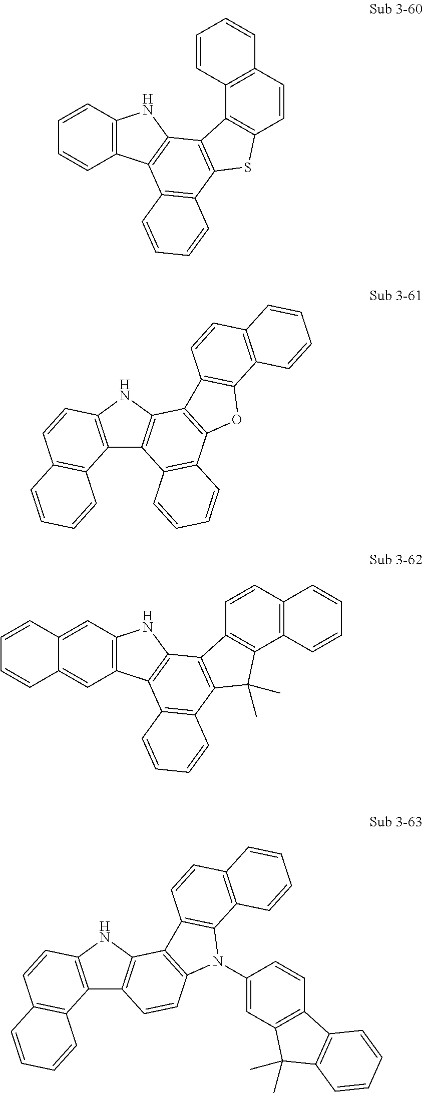 [SMILES: c1ccc2c(c1)ccc1sc3c4ccccc4c4c5ccccc5[nH]c4c3c12 ; c1ccc2c(c1)ccc1c2oc2c3ccccc3c3c([nH]c4ccc5ccccc5c43)c12 ; CC1(C)c2c(ccc3ccccc23)-c2c1c1ccccc1c1c2[nH]c2cc3ccccc3cc21 ; CC1(C)c2ccccc2-c2ccc(-n3c4ccc5c([nH]c6ccc7ccccc7c65)c4c4ccc5ccccc5c43)cc21]

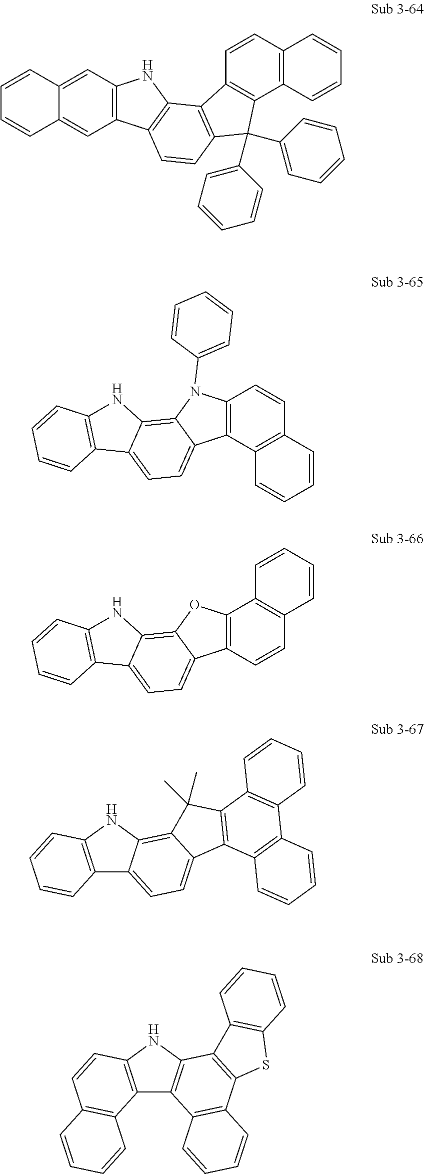

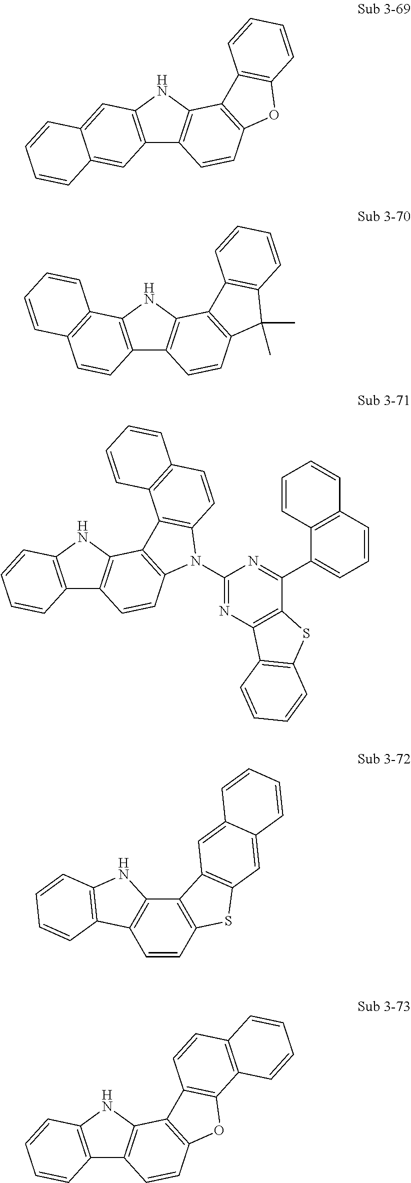 [SMILES: c1ccc2cc3c(cc2c1)[nH]c1c3ccc2oc3ccccc3c21 ; CC1(C)c2ccccc2-c2c1ccc1c2[nH]c2c3ccccc3ccc12 ; c1ccc2c(-c3nc(-n4c5ccc6ccccc6c5c5c6[nH]c7ccccc7c6ccc54)nc4c3sc3ccccc34)cccc2c1 ; c1ccc2cc3c(cc2c1)sc1ccc2c4ccccc4[nH]c2c13 ; c1ccc2c(c1)ccc1c2oc2ccc3c4ccccc4[nH]c3c21]

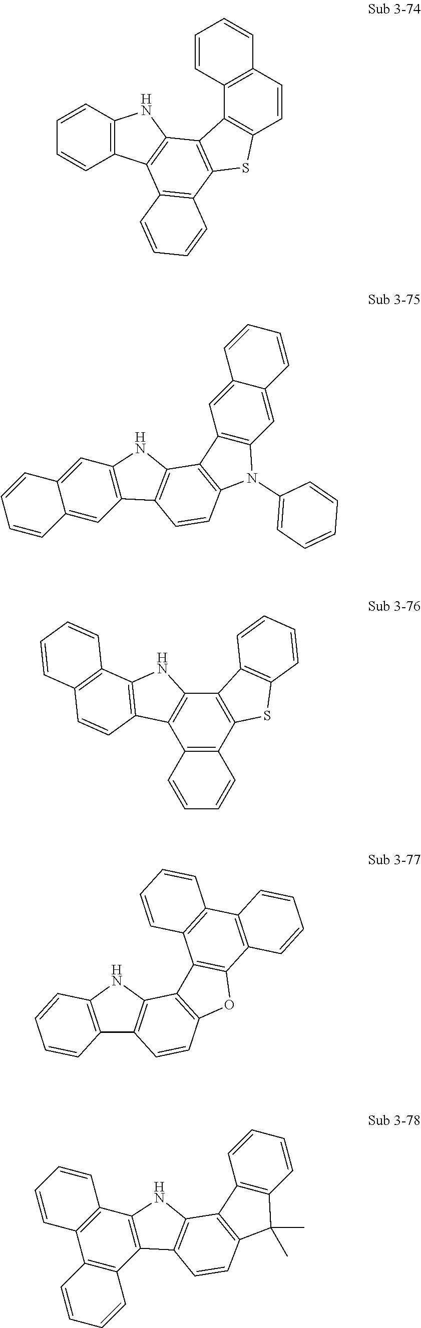 [SMILES: c1ccc2c(c1)ccc1sc3c4ccccc4c4c5ccccc5[nH]c4c3c12 ; c1ccc(-n2c3cc4ccccc4cc3c3c4[nH]c5cc6ccccc6cc5c4ccc32)cc1 ; c1ccc2c(c1)ccc1c2[nH]c2c1c1ccccc1c1sc3ccccc3c21 ; c1ccc2c(c1)[nH]c1c2ccc2oc3c4ccccc4c4ccccc4c3c21 ; CC1(C)c2ccccc2-c2c1ccc1c2[nH]c2c3ccccc3c3ccccc3c12]

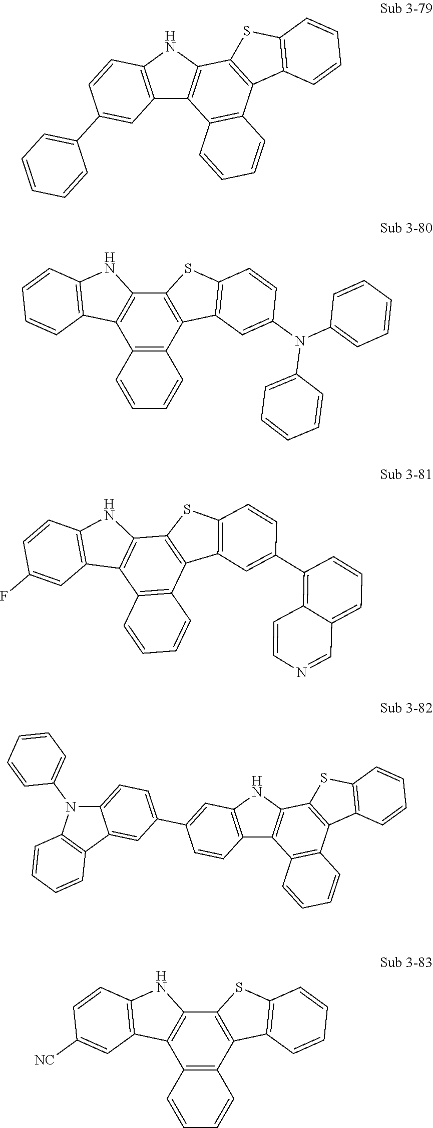 [SMILES: c1ccc(-c2ccc3[nH]c4c5sc6ccccc6c5c5ccccc5c4c3c2)cc1 ; c1ccc(N(c2ccccc2)c2ccc3sc4c5[nH]c6ccccc6c5c5ccccc5c4c3c2)cc1 ; Fc1ccc2[nH]c3c4sc5ccc(-c6cccc7cnccc67)cc5c4c4ccccc4c3c2c1 ; c1ccc(-n2c3ccccc3c3cc(-c4ccc5c(c4)[nH]c4c6sc7ccccc7c6c6ccccc6c54)ccc32)cc1 ; N#Cc1ccc2[nH]c3c4sc5ccccc5c4c4ccccc4c3c2c1]

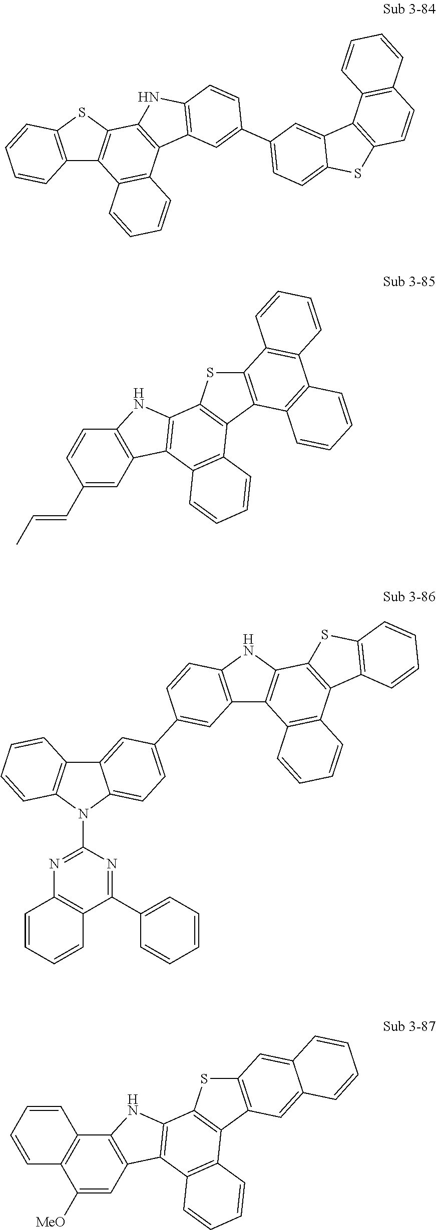 [SMILES: c1ccc2c(c1)ccc1sc3ccc(-c4ccc5[nH]c6c7sc8ccccc8c7c7ccccc7c6c5c4)cc3c12 ; C/C=C/c1ccc2[nH]c3c4sc5c6ccccc6c6ccccc6c5c4c4ccccc4c3c2c1 ; c1ccc(-c2nc(-n3c4ccccc4c4cc(-c5ccc6[nH]c7c8sc9ccccc9c8c8ccccc8c7c6c5)ccc43)nc3ccccc23)cc1 ; COc1cc2c([nH]c3c4sc5cc6ccccc6cc5c4c4ccccc4c23)c2ccccc12]

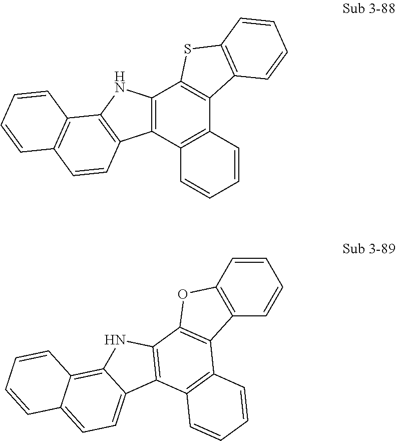 [SMILES: c1ccc2c(c1)ccc1c2[nH]c2c3sc4ccccc4c3c3ccccc3c12 ; c1ccc2c(c1)ccc1c2[nH]c2c3oc4ccccc4c3c3ccccc3c12]

TABLE 4

| compound | FD-MS | compound | FD-MS |
|---|---|---|---|
| Sub 3-1 | m/z = 323.08(C₂₂H₁₃NS = 323.41) | Sub 3-2 | m/z = 323.08(C₂₂H₁₃NS = 323.41) |
| Sub 3-3 | m/z = 307.10(C₂₂H₁₃NO = 307.34) | Sub 3-4 | m/z = 307.10(C₂₂H₁₃NO = 307.34) |
| Sub 3-5 | m/z = 333.15(C₂₅H₁₉N = 333.43) | Sub 3-6 | m/z = 382.15(C₂₈H₁₈N₂ = 382.46) |
| Sub 3-7 | m/z = 382.15(C₂₈H₁₈N₂ = 382.46) | Sub 3-8 | m/z = 323.08(C₂₂H₁₃NS = 323.41) |
| Sub 3-9 | m/z = 307.10(C₂₂H₁₃NO = 307.34) | Sub 3-10 | m/z = 383.17(C₂₉H₂₁N = 383.49) |
| Sub 3-11 | m/z = 373.09(C₂₆H₁₅NS = 373.47) | Sub 3-12 | m/z = 323.08(C₂₂H₁₃NS = 323.41) |
| Sub 3-13 | m/z = 357.12(C₂₆H₁₅NO = 357.41) | Sub 3-14 | m/z = 333.15(C₂₅H₁₉N = 333.43) |
| Sub 3-15 | m/z = 373.09(C₂₆H₁₅NS = 373.47) | Sub 3-16 | m/z = 357.12(C₂₆H₁₅NO = 357.41) |
| Sub 3-17 | m/z = 383.17(C₂₉H₂₁N = 383.49) | Sub 3-18 | m/z = 373.09(C₂₆H₁₅NS = 373.47) |
| Sub 3-19 | m/z = 407.13(C₃₀H₁₇NO = 407.47) | Sub 3-20 | m/z = 433.18(C₃₃H₂₃N = 433.55) |
| Sub 3-21 | m/z = 373.09(C₂₆H₁₅NS = 373.47) | Sub 3-22 | m/z = 357.12(C₂₆H₁₅NO = 357.41) |
| Sub 3-23 | m/z = 662.25(C₄₈H₃₀N₄ = 662.80) | Sub 3-24 | m/z = 373.09(C₂₆H₁₅NS = 373.47) |
| Sub 3-25 | m/z = 357.12(C₂₆H₁₅NO = 357.41) | Sub 3-26 | m/z = 383.17(C₂₉H₂₁N = 383.49) |
| Sub 3-27 | m/z = 432.16(C₃₂H₂₀N₂ = 432.53) | Sub 3-28 | m/z = 323.08(C₂₂H₁₃NS = 323.41) |
| Sub 3-29 | m/z = 307.10(C₂₂H₁₃NO = 307.35) | Sub 3-30 | m/z = 455.17(C₃₅H₂₁N = 455.56) |
| Sub 3-31 | m/z = 432.16(C₃₂H₂₀N₂ = 432.53) | Sub 3-32 | m/z = 373.09(C₂₆H₁₅NS = 373.47) |
| Sub 3-33 | m/z = 357.12(C₂₆H₁₅NO = 357.41) | Sub 3-34 | m/z = 383.17(C₂₉H₂₁N = 383.49) |
| Sub 3-35 | m/z = 432.16(C₃₂H₂₀N₂ = 432.53) | Sub 3-36 | m/z = 373.09(C₂₆H₁₅NS = 373.47) |
| Sub 3-37 | m/z = 357.12(C₂₆H₁₅NO = 357.41) | Sub 3-38 | m/z = 383.17(C₂₉H₂₁N = 383.49) |
| Sub 3-39 | m/z = 432.16(C₃₂H₂₀N₂ = 432.53) | Sub 3-40 | m/z = 373.09(C₂₆H₁₅NS = 373.47) |
| Sub 3-41 | m/z = 357.12(C₂₆H₁₅NO = 357.41) | Sub 3-42 | m/z = 505.18(C₃₉H₂₃N = 505.62) |
| Sub 3-43 | m/z = 382.15(C₂₈H₁₈N₂ = 382.47) | Sub 3-44 | m/z = 323.08(C₂₂H₁₃NS = 323.41) |
| Sub 3-45 | m/z = 307.10(C₂₂H₁₃NO = 307.35) | Sub 3-46 | m/z = 333.15(C₂₅H₁₉N = 333.43) |
| Sub 3-47 | m/z = 432.16(C₃₂H₂₀N₂ = 439.53) | Sub 3-48 | m/z = 323.08(C₂₂H₁₃NS = 323.41) |
| Sub 3-49 | m/z = 307.10(C₂₂H₁₃NO = 307.35) | Sub 3-50 | m/z = 457.18(C₃₅H₂₃N = 457.58) |
| Sub 3-51 | m/z = 432.16(C₃₂H₂₀N₂ = 432.53) | Sub 3-52 | m/z = 373.09(C₂₆H₁₅NS = 373.47) |
| Sub 3-53 | m/z = 357.12(C₂₆H₁₅NO = 357.41) | Sub 3-54 | m/z = 383.17(C₂₉H₂₁N = 383.49) |
| Sub 3-55 | m/z = 433.16(C₃₁H₁₉N₃ = 433.51) | Sub 3-56 | m/z = 356.13(C₂₆H₁₆N₂ = 356.43) |
| Sub 3-57 | m/z = 357.12(C₂₆H₁₅NO = 357.41) | Sub 3-58 | m/z = 383.17(C₂₉H₂₁N = 383.49) |
| Sub 3-59 | m/z = 538.15(C₃₈H₂₂N₂S = 538.67) | Sub 3-60 | m/z = 373.09(C₂₆H₁₅NS = 373.47) |
| Sub 3-61 | m/z = 407.13(C₃₀H₁₇NO = 407.47) | Sub 3-62 | m/z = 433.18(C₃₃H₂₃N = 433.55) |
| Sub 3-63 | m/z = 548.23(C₄₁H₂₈N₂ = 548.69) | Sub 3-64 | m/z = 507.20(C₃₉H₂₅N = 507.64) |
| Sub 3-65 | m/z = 382.15(C₂₈H₁₈N₂ = 382.47) | Sub 3-66 | m/z = 307.10(C₂₂H₁₃NO = 307.35) |
| Sub 3-67 | m/z = 383.17(C₂₉H₂₁N = 383.49) | Sub 3-68 | m/z = 373.09(C₂₆H₁₅NS = 373.47) |
| Sub 3-69 | m/z = 307.10(C₂₂H₁₃NO = 307.35) | Sub 3-70 | m/z = 333.15(C₂₅H₁₉N = 333.43) |
| Sub 3-71 | m/z = 616.17(C₄₂H₂₄N₄S = 616.74) | Sub 3-72 | m/z = 323.08(C₂₂H₁₃NS = 323.41) |
| Sub 3-73 | m/z = 307.10(C₂₂H₁₃NO = 307.35) | Sub 3-74 | m/z = 373.09(C₂₆H₁₅NS = 373.47) |
| Sub 3-75 | m/z = 432.16(C₃₂H₂₀N₂ = 432.53) | Sub 3-76 | m/z = 373.09(C₂₆H₁₅NS = 373.47) |
| Sub 3-77 | m/z = 357.12(C₂₆H₁₅NO = 357.41) | Sub 3-78 | m/z = 383.17(C₂₉H₂₁N = 383.49) |
| Sub 3-79 | m/z = 399.11(C₂₈H₁₇NS = 399.51) | Sub 3-80 | m/z = 490.15(C₃₄H₂₂N₂S = 490.62) |
| Sub 3-81 | m/z = 468.11(C₃₁H₁₇FN₂S = 468.55) | Sub 3-82 | m/z = 564.17(C₄₀H₂₄N₂S = 564.71) |
| Sub 3-83 | m/z = 348.07(C₂₃H₁₂N₂S = 348.42) | Sub 3-84 | m/z = 555.11(C₃₈H₂₁NS₂ = 555.71) |
| Sub 3-85 | m/z = 463.14(C₃₃H₂₁NS = 463.60) | Sub 3-86 | m/z = 692.20(C₄₈H₂₈N₄S = 692.84) |
| Sub 3-87 | m/z = 437.14(C₃₁H₁₉NO₂ = 437.50) | Sub 3-88 | m/z = 373.09(C₂₆H₁₅NS = 373.47) |
| Sub 3-89 | m/z = 357.12(C₂₆H₁₅NO = 357.41) | | |

### Synthesis Example of Sub 4

Sub 4 of Scheme 6 can be synthesized by the reaction path of Scheme 7 below, but is not limited thereto.

At this time, Hal⁵=I, Br, C₁; Hal⁶=Br, C₁

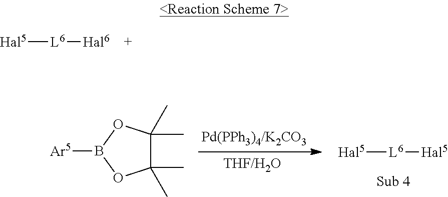

### Synthesis of Sub 4-35

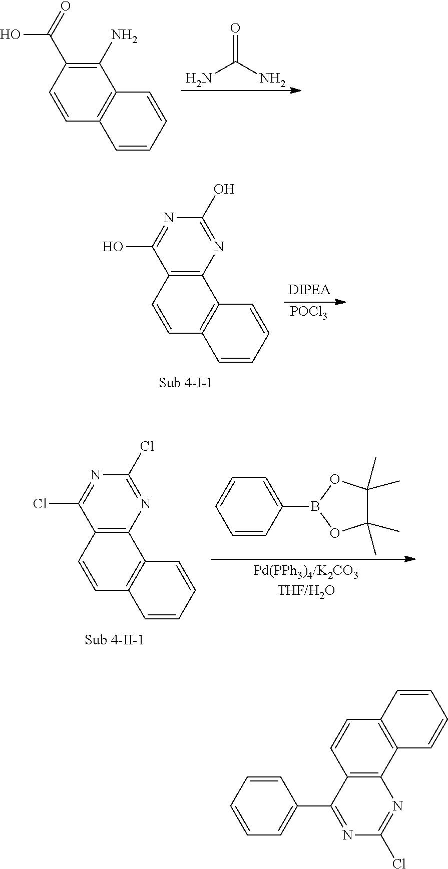

### (1) Synthesis of Sub 4-I-1

The starting material, 1-amino-2-naphthoic acid (CAS Registry Number: 4919-43-1) (75.11 g, 401.25 mmol), was placed in a round bottom flask with urea (CAS Registry Number: 4919-43-1) (168.69 g, 2808.75 mmol) and stirred at 160° C. After confirming the reaction by TLC, the reaction mixture was cooled to 100° C., water (200 ml) was added, and the mixture was stirred for 1 hour. When the reaction was completed, the resulting solid was filtered under reduced pressure, washed with water, and then dried to obtain 63.86 g (yield: 75%) of the product.

### (2) Synthesis of Sub 4-II-1

Sub 4-I-1 (63.86 g, 300.94 mmol) was dissolved in POCl₃ (200 ml) at room temperature in a round bottom flask, and N, N-Diisopropylethylamine (97.23 g, 752.36 mmol) was slowly added dropwise thereto, followed by stirring at 90° C. After the reaction was completed, the reaction mixture was concentrated, and then ice water (500 ml) was added thereto, followed by stirring at room temperature for 1 hour. The resulting solid was filtered under reduced pressure and dried to obtain 67.47 g (yield: 90%) of the product.

### (3) Synthesis of Sub 4-35

After Sub 4-II-1 (67.47 g, 270.86 mmol) was dissolved in THF (950 ml) in a round bottom flask, 4,4,5,5-tetramethyl-2-phenyl-1,3,2-dioxaborolane (CAS Registry Number: 24388-23-6) (60.80 g, 297.94 mmol), Pd(PPh₃)₄ (12.52 g, 10.83 mmol), K₂CO₃ (112.30 g, 812.57 mmol) and water (475 mL) were added to dissolve and stirred at 90° C. After the reaction was completed, the reaction mixture was extracted with CH₂Cl₂ and water. The organic layer was dried over MgSO₄ and concentrated. The resulting compound was purified by silicagel column and recrystallized to obtain 44.89 g (yield: 57%) of the product.

### Synthesis of Sub 4-40

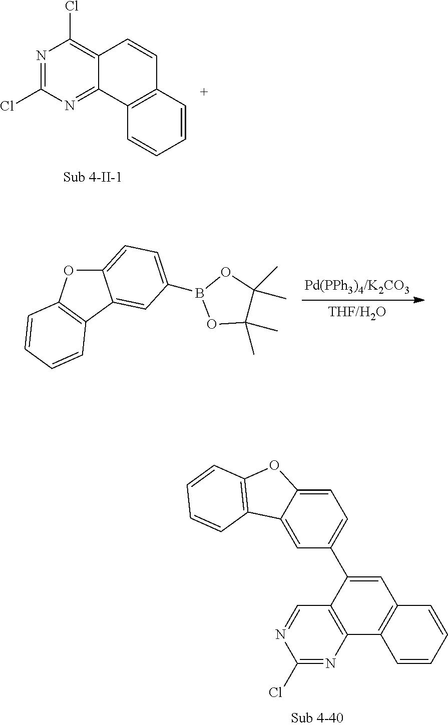 [SMILES: Clc1nc(Cl)c2ccc3ccccc3c2n1 ; CC1(C)OB(c2ccc3oc4ccccc4c3c2)OC1(C)C ; Clc1ncc2c(-c3ccc4oc5ccccc5c4c3)cc3ccccc3c2n1]

Sub 4-II-1 (19 g, 76.28 mmol), 2-(dibenzo[b,d]furan-2-yl)-4,4,5,5-tetramethyl-1,3,2-dioxaborolane (CAS Registry Number: 947770-80-1) (22.44 g, 76.28 mmol), Pd(PPh₃)₄ (1.32 g, 1.14 mmol), K₂CO₃ (15.81 g, 114.42 mmol), THF (336 ml) and water (168 ml) were added and carried out in the same manner as in Sub 4-35 to give the product. (15.69 g, 54%).

### Synthesis of Sub 4-43

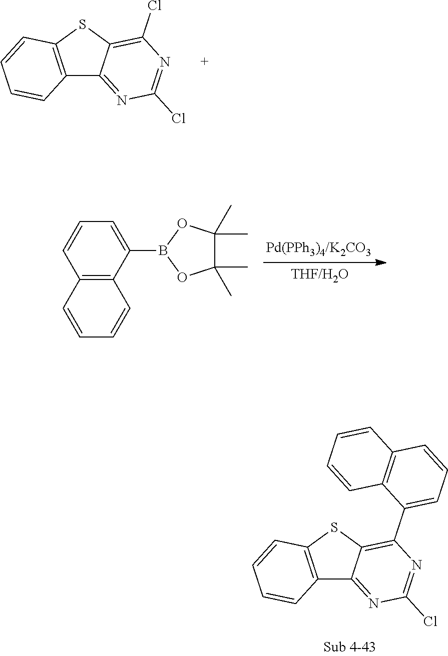 [SMILES: Clc1nc(Cl)c2sc3ccccc3c2n1 ; CC1(C)OB(c2cccc3ccccc23)OC1(C)C ; Clc1nc(-c2cccc3ccccc23)c2sc3ccccc3c2n1]

2,4-dichlorobenzo[4,5]thieno[3,2-d]pyrimidine (CAS Registry Number: 160199-05-3) (32.01 g, 125.47 mmol), 4,4,5,5-tetramethyl-2-(naphthalen-1-yl)-1,3,2-dioxaborolane (CAS Registry Number: 68716-52-9) (35.07 g, 138.02 mmol), Pd(PPh₃)₄ (5.80 g, 5.02 mmol), K₂CO₃ (52.02 g, 376.41 mmol), THF (440 ml) and water (220 ml) were added and carried out in the same manner as in Sub 4-35 to give the product. (19.58 g, 45%).

The compounds belonging to Sub 4 may be, but not limited to, the following compounds, and Table 5 shows FD-MS (Field Desorption-Mass Spectrometry) values of Sub 4 compounds.

Examples of Sub 4 include, but are not limited to, the followings.

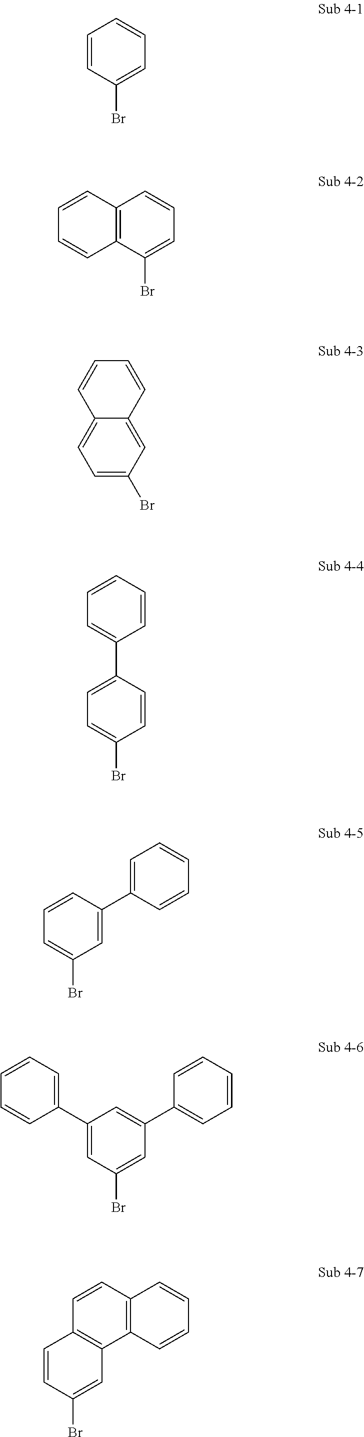

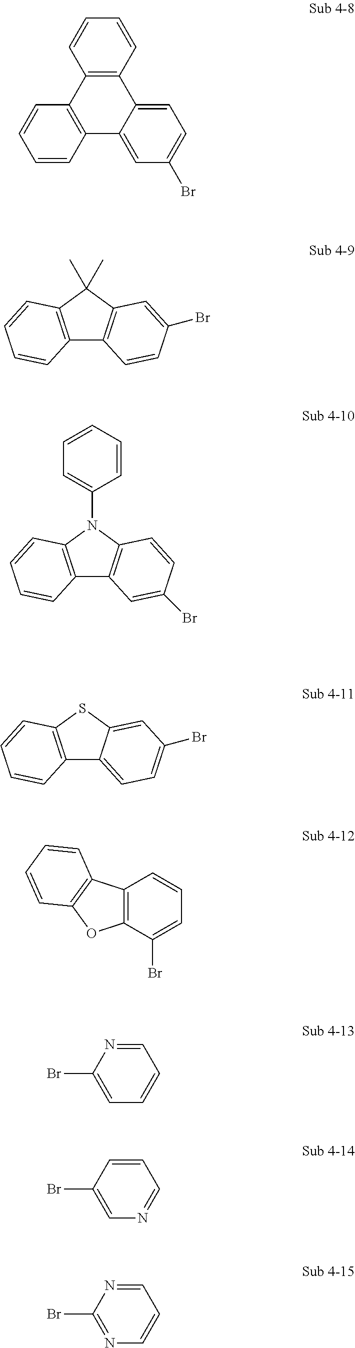

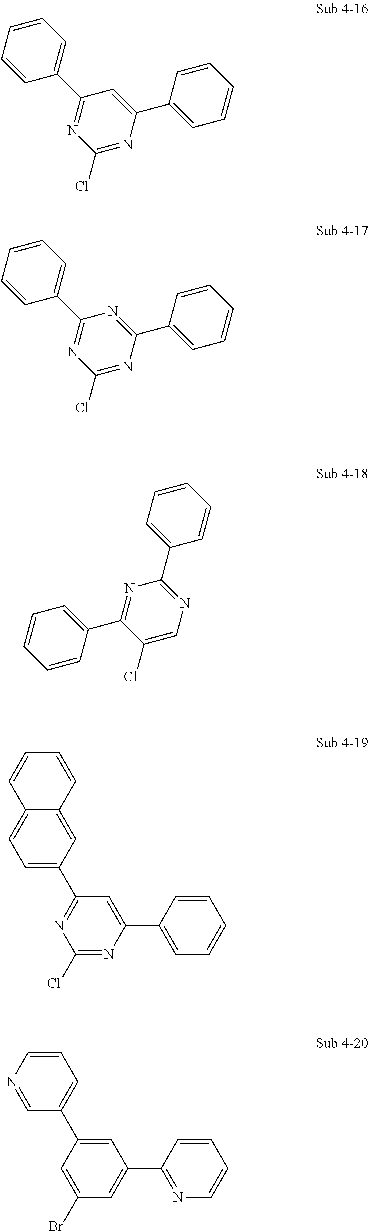 [SMILES: Clc1nc(-c2ccccc2)cc(-c2ccccc2)n1 ; Clc1nc(-c2ccccc2)nc(-c2ccccc2)n1 ; Clc1cnc(-c2ccccc2)nc1-c1ccccc1 ; Clc1nc(-c2ccccc2)cc(-c2ccc3ccccc3c2)n1 ; Brc1cc(-c2cccnc2)cc(-c2ccccn2)c1]

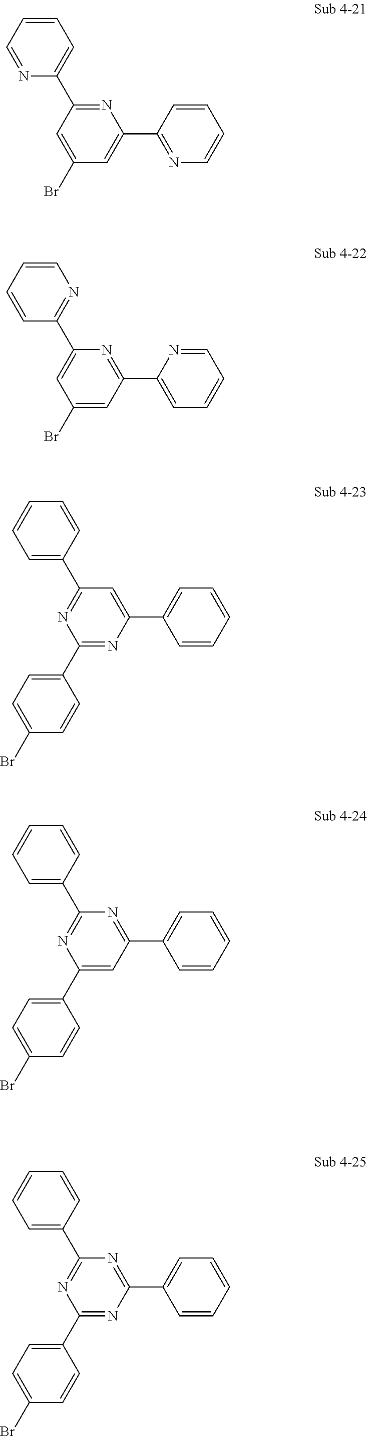 [SMILES: Brc1cc(-c2ccccn2)nc(-c2ccccn2)c1 ; Brc1cc(-c2ccccn2)nc(-c2ccccn2)c1 ; Brc1ccc(-c2nc(-c3ccccc3)cc(-c3ccccc3)n2)cc1 ; Brc1ccc(-c2cc(-c3ccccc3)nc(-c3ccccc3)n2)cc1 ; Brc1ccc(-c2nc(-c3ccccc3)nc(-c3ccccc3)n2)cc1]

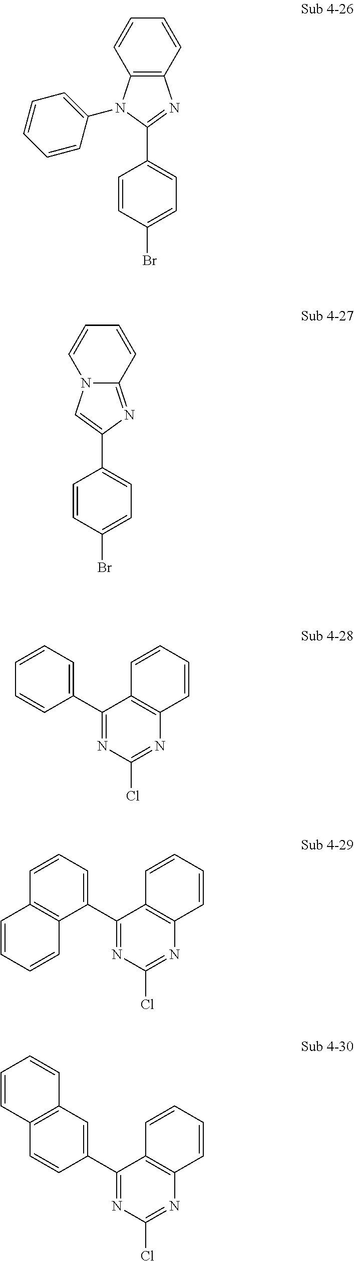 [SMILES: Brc1ccc(-c2nc3ccccc3n2-c2ccccc2)cc1 ; Brc1ccc(-c2cn3ccccc3n2)cc1 ; Clc1nc(-c2ccccc2)c2ccccc2n1 ; Clc1nc(-c2cccc3ccccc23)c2ccccc2n1 ; Clc1nc(-c2ccc3ccccc3c2)c2ccccc2n1]

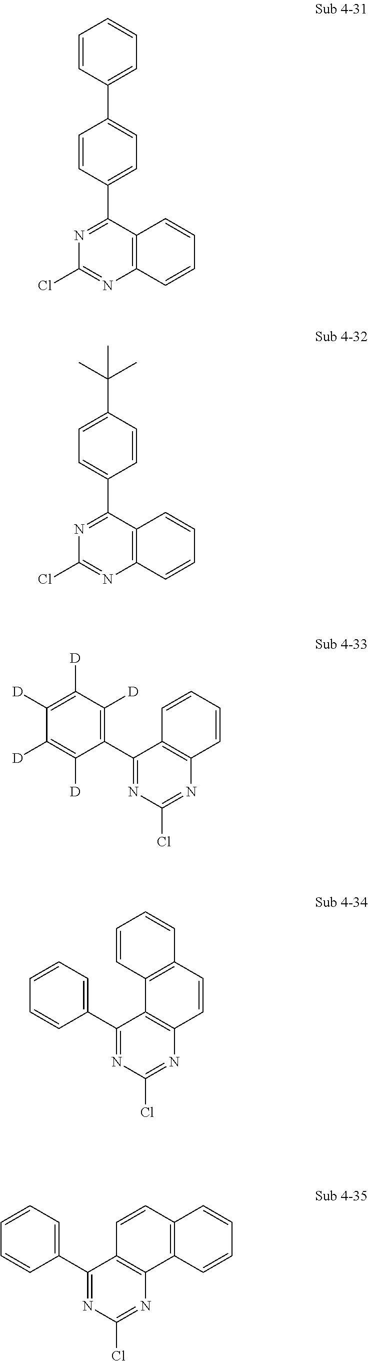 [SMILES: Clc1nc(-c2ccc(-c3ccccc3)cc2)c2ccccc2n1 ; CC(C)(C)c1ccc(-c2nc(Cl)nc3ccccc23)cc1 ; [2H]c1c([2H])c([2H])c(-c2nc(Cl)nc3ccccc23)c([2H])c1[2H] ; Clc1nc(-c2ccccc2)c2c(ccc3ccccc32)n1 ; Clc1nc(-c2ccccc2)c2ccc3ccccc3c2n1]

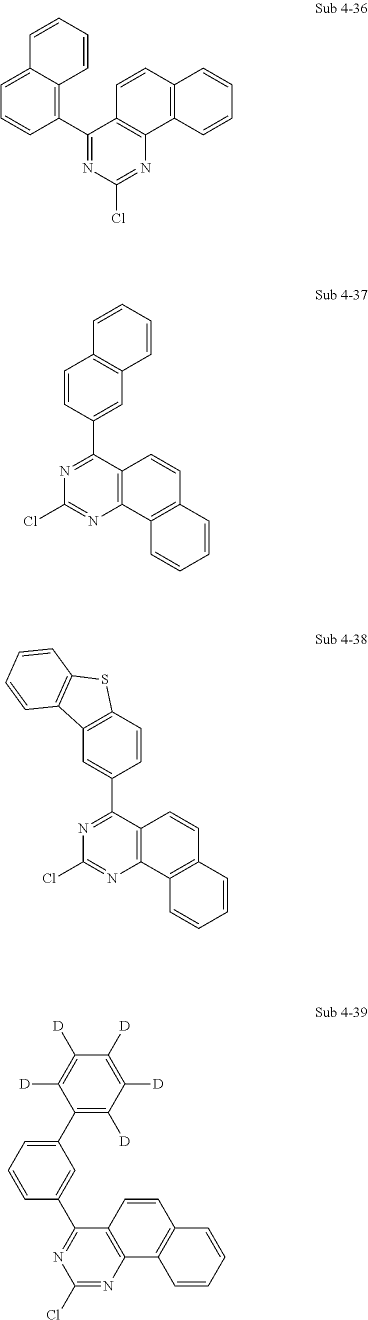 [SMILES: Clc1nc(-c2cccc3ccccc23)c2ccc3ccccc3c2n1 ; Clc1nc(-c2ccc3ccccc3c2)c2ccc3ccccc3c2n1 ; Clc1nc(-c2ccc3sc4ccccc4c3c2)c2ccc3ccccc3c2n1 ; [2H]c1c([2H])c([2H])c(-c2cccc(-c3nc(Cl)nc4c3ccc3ccccc34)c2)c([2H])c1[2H]]

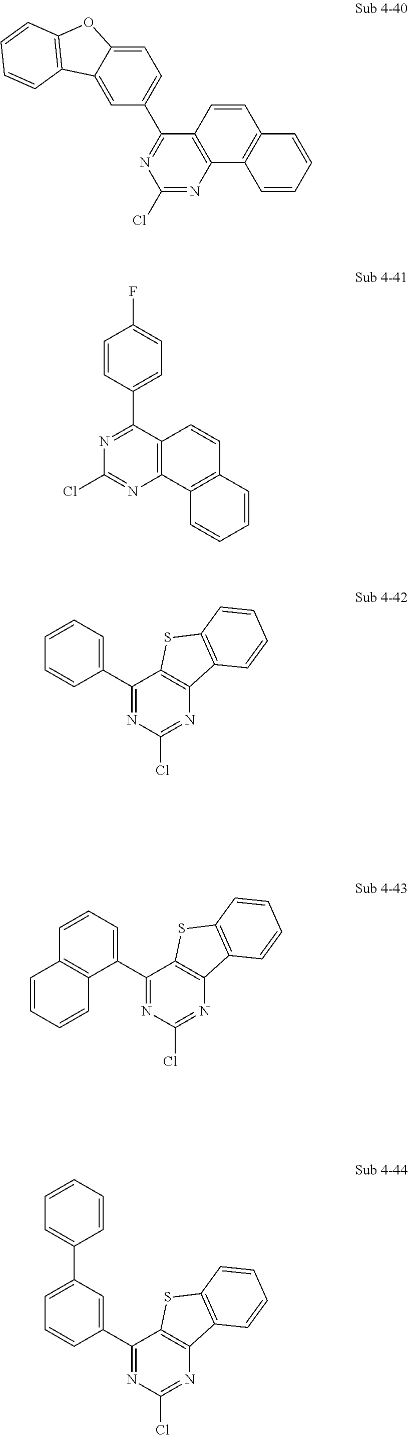 [SMILES: Clc1nc(-c2ccc3oc4ccccc4c3c2)c2ccc3ccccc3c2n1 ; Fc1ccc(-c2nc(Cl)nc3c2ccc2ccccc23)cc1 ; Clc1nc(-c2ccccc2)c2sc3ccccc3c2n1 ; Clc1nc(-c2cccc3ccccc23)c2sc3ccccc3c2n1 ; Clc1nc(-c2cccc(-c3ccccc3)c2)c2sc3ccccc3c2n1]

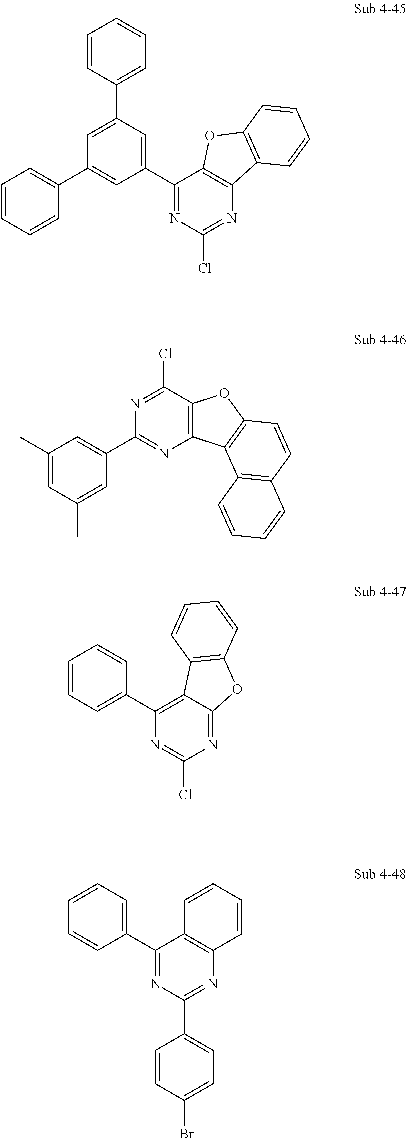 [SMILES: Clc1nc(-c2cc(-c3ccccc3)cc(-c3ccccc3)c2)c2oc3ccccc3c2n1 ; Cc1cc(C)cc(-c2nc(Cl)c3oc4ccc5ccccc5c4c3n2)c1 ; Clc1nc(-c2ccccc2)c2c(n1)oc1ccccc12 ; Brc1ccc(-c2nc(-c3ccccc3)c3ccccc3n2)cc1]

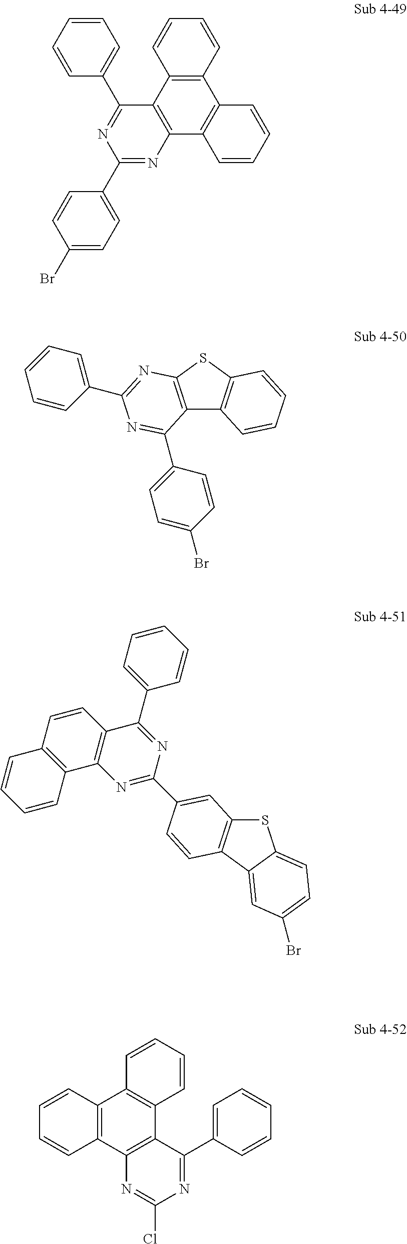 [SMILES: Brc1ccc(-c2nc(-c3ccccc3)c3c4ccccc4c4ccccc4c3n2)cc1 ; Brc1ccc(-c2nc(-c3ccccc3)nc3sc4ccccc4c23)cc1 ; Brc1ccc2sc3cc(-c4nc(-c5ccccc5)c5ccc6ccccc6c5n4)ccc3c2c1 ; Clc1nc(-c2ccccc2)c2c3ccccc3c3ccccc3c2n1]

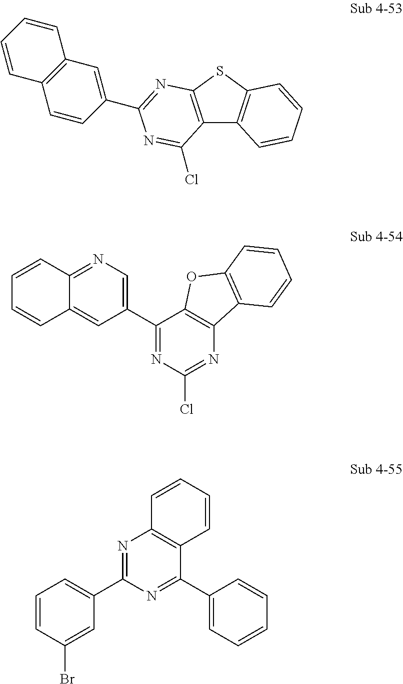 [SMILES: Clc1nc(-c2ccc3ccccc3c2)nc2sc3ccccc3c12 ; Clc1nc(-c2cnc3ccccc3c2)c2oc3ccccc3c2n1 ; Brc1cccc(-c2nc(-c3ccccc3)c3ccccc3n2)c1]

TABLE 5

| compound | FD-MS | compound | FD-MS |
|---|---|---|---|
| Sub 4-1 | m/z = 155.96(C₆H₅Br = 157.01) | Sub 4-2 | m/z = 205.97(C₁₀H₇Br = 207.07) |
| Sub 4-3 | m/z = 205.97(C₁₀H₇Br = 207.07) | Sub 4-4 | m/z = 231.99(C₁₂H₉Br = 233.11) |
| Sub 4-5 | m/z = 231.99(C₁₂H₉Br = 233.11) | Sub 4-6 | m/z = 308.02(C₁₈H₁₃Br = 309.21) |
| Sub 4-7 | m/z = 255.99(C₁₄H₉Br = 257.13) | Sub 4-8 | m/z = 306.00(C₁₈H₁₁Br = 307.19) |
| Sub 4-9 | m/z = 272.02(C₁₅H₁₃Br = 273.17) | Sub 4-10 | m/z = 321.02(C₁₈H₁₂BrN = 322.21) |
| Sub 4-11 | m/z = 261.95(C₁₂H₇BrS = 263.15) | Sub 4-12 | m/z = 245.97(C₁₂H₇BrO = 247.09) |
| Sub 4-13 | m/z = 156.95(C₅H₄BrN = 158.00) | Sub 4-14 | m/z = 156.95(C₅H₄BrN = 158.00) |
| Sub 4-15 | m/z = 157.95(C₄H₃BrN₂ = 158.99) | Sub 4-16 | m/z = 266.06(C₁₆H₁₁ClN₂ = 266.72) |
| Sub 4-17 | m/z = 267.06(C₁₅H₁₀ClN₃ = 267.72) | Sub 4-18 | m/z = 266.06(C₁₆H₁₁ClN₂ = 266.72) |
| Sub 4-19 | m/z = 316.08(C₂₀H₁₃ClN₂ = 316.79) | Sub 4-20 | m/z = 310.01(C₁₆H₁₁BrN₂ = 311.18) |
| Sub 4-21 | m/z = 311.01(C₁₅H₁₀BrN₃ = 312.17) | Sub 4-22 | m/z = 311.01(C₁₅H₁₀BrN₃ = 312.17) |
| Sub 4-23 | m/z = 386.04(C₂₂H₁₅BrN₂ = 387.28) | Sub 4-24 | m/z = 386.04(C₂₂H₁₅BrN₂ = 387.28) |
| Sub 4-25 | m/z = 387.04(C₂₁H₁₄BrN₃ = 388.27) | Sub 4-26 | m/z = 348.03(C₁₉H₁₃BrN₂ = 349.23) |
| Sub 4-27 | m/z = 273.13(C₁₃H₉BrN₂ = 273.13) | Sub 4-28 | m/z = 240.05(C₁₄H₉ClN₂ = 240.69 |
| Sub 4-29 | m/z = 290.06(C₁₈H₁₁ClN₂ = 290.75) | Sub 4-30 | m/z = 290.06(C₁₈H₁₁ClN₂ = 290.75) |
| Sub 4-31 | m/z = 316.08(C₂₀H₁₃ClN₂ = 316.79) | Sub 4-32 | m/z = 296.11(C₁₈H₁₇ClN₂ = 296.80) |
| Sub 4-33 | m/z = 245.08(C₁₄H₄D₅ClN₂ = 245.72) | Sub 4-34 | m/z = 290.06(C₁₈H₁₁ClN₂ = 290.75) |
| Sub 4-35 | m/z = 290.06(C₁₈H₁₁ClN₂ = 290.75) | Sub 4-36 | m/z = 340.08(C₂₂H₁₃ClN₂ = 340.81) |
| Sub 4-37 | m/z = 340.08(C₂₂H₁₃ClN₂ = 340.81) | Sub 4-38 | m/z = 396.05(C₂₄H₁₃ClN₂S = 396.89) |
| Sub 4-39 | m/z = 371.12(C₂₄H₁₀D₅ClN₂ = 371.88) | Sub 4-40 | m/z = 380.07(C₂₄H₁₃ClN₂O = 380.83) |
| Sub 4-41 | m/z = 308.05(C₁₈H₁₀ClFN₂ = 308.74) | Sub 4-42 | m/z = 296.02(C₁₆H₉ClN₂S = 296.77) |
| Sub 4-43 | m/z = 346.03(C₂₀H₁₁ClN₂S = 346.83) | Sub 4-44 | m/z = 372.05(C₂₂H₁₃ClN₂S = 372.87) |
| Sub 4-45 | m/z = 432.10(C₂₈H₁₇ClN₂O = 432.91) | Sub 4-46 | m/z = 358.09(C₂₂H₁₅ClN₂O = 358.83) |
| Sub 4-47 | m/z = 280.04(C₁₆H₉ClN₂O = 280.71) | Sub 4-48 | m/z = 360.03(C₂₀H₁₃BrN₂ = 361.24) |
| Sub 4-49 | m/z = 460.06(C₂₈H₁₇BrN₂ = 461.36) | Sub 4-50 | m/z = 416.00(C₂₂H₁₃BrN₂S = 417.32) |
| Sub 4-51 | m/z = 516.03(C₃₀H₁₇BrN₂S = 517.44) | Sub 4-52 | m/z = 340.08(C₂₂H₁₃ClN₂ = 340.81) |
| Sub 4-53 | m/z = 346.03(C₂₀H₁₁ClN₂S = 346.83) | Sub 4-54 | m/z = 331.05(C₁₉H₁₀ClN₃O = 331.76) |
| Sub 4-55 | m/z = 360.03(C₂₀H₁₃BrN₂ = 361.24) | | |

### Synthesis of Final products 2

After Sub 3 (1 eq.) was dissolved in toluene in a round bottom flask, Sub 4 (1.1 eq.), Pd₂(dba)₃ (0.03 eq.), P(t-Bu)₃ (0.1 eq.), NaOt-Bu (3 eq.) were added and stirred at 100° C. When the reaction was complete, the reaction mixture was extracted with CH₂Cl₂ and water. The organic layer was dried over MgSO₄ and concentrated. The resulting compound was separated by silicagel column chromatography and recrystallization to obtain the Final products.

### Synthesis Example of 3-1

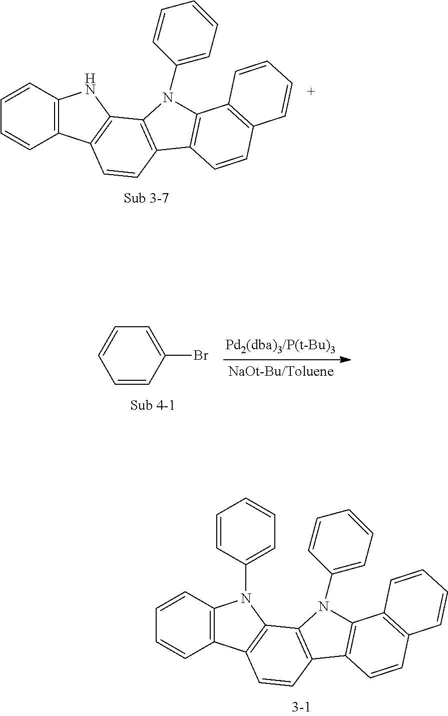 [SMILES: c1ccc(-n2c3c4ccccc4ccc3c3ccc4c5ccccc5[nH]c4c32)cc1 ; Brc1ccccc1 ; c1ccc(-n2c3ccccc3c3ccc4c5ccc6ccccc6c5n(-c5ccccc5)c4c32)cc1]

Sub 3-7 (11 g, 28.76 mmol) was dissolved in toluene (302 mL) in a round bottom flask, and Sub 4-1 (4.52 g, 28.76 mmol), Pd₂(dba)₃ (0.79 g, 0.86 mmol), P(t-Bu)₃ (0.35 g, 1.73 mmol), NaOt-Bu (8.29 g, 86.28 mmol) were added and stirred at 100° C. After the reaction was completed, the reaction mixture was extracted with CH₂Cl₂ and water. The organic layer was dried over MgSO₄ and concentrated. The resulting compound was separated by silicagel column chromatography and recrystallized to obtain 9.50 g of the product. (Yield: 72%)

### Synthesis Example of 3-6

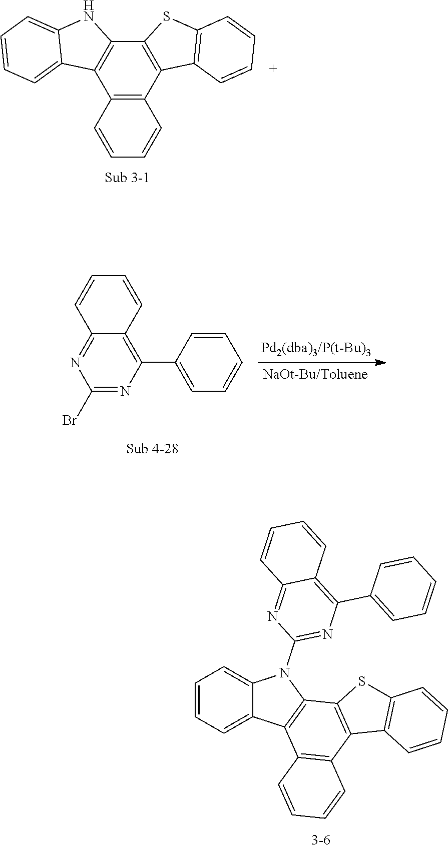 [SMILES: c1ccc2c(c1)[nH]c1c3sc4ccccc4c3c3ccccc3c21 ; Brc1nc(-c2ccccc2)c2ccccc2n1 ; c1ccc(-c2nc(-n3c4ccccc4c4c5ccccc5c5c6ccccc6sc5c43)nc3ccccc23)cc1]

Sub 3-1 (15.3 g, 47.3 mmol), toluene (500 mL), Sub 4-28 (14.8 g, 52.0 mmol), Pd₂(dba)₃ (1.3 g, 1.42 mmol), P(t-Bu)₃ (0.57 g, 2.84 mmol), NaOt-Bu (13.64 g, 141.93 mmol) were carried out in the same manner as in 3-1 to give the product. (18.97 g, 72%).

### Synthesis Example of 3-7

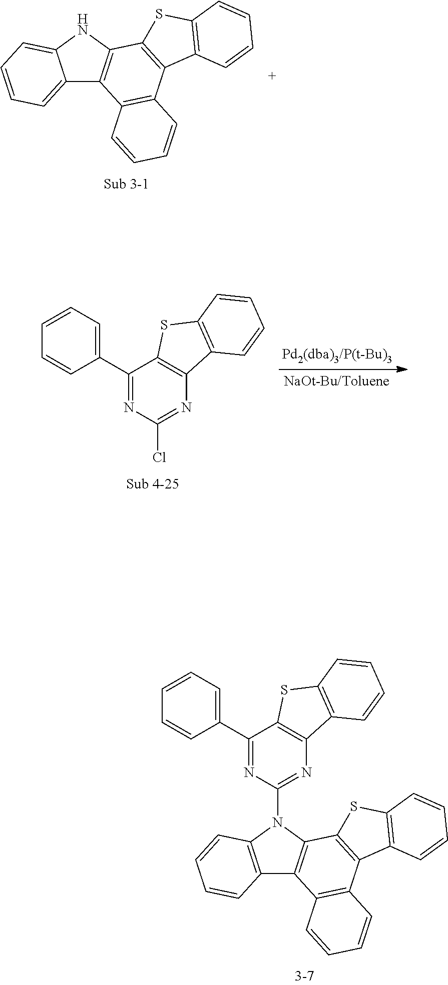 [SMILES: c1ccc2c(c1)[nH]c1c3sc4ccccc4c3c3ccccc3c21 ; Clc1nc(-c2ccccc2)c2sc3ccccc3c2n1 ; c1ccc(-c2nc(-n3c4ccccc4c4c5ccccc5c5c6ccccc6sc5c43)nc3c2sc2ccccc23)cc1]

Sub 3-1 (10 g, 30.92 mmol), toluene (325 ml), Sub 4-25 (10.0 g, 34.01 mmol), Pd₂(dba)₃ (0.85 g, 0.93 mmol), P(t-Bu)₃ (0.38 g, 1.86 mmol), NaOt-Bu (8.91 g, 92.76 mmol) were carried out in the same manner as in 3-1 to give the product. (12.81 g, 71%).

### Synthesis Example of 3-8

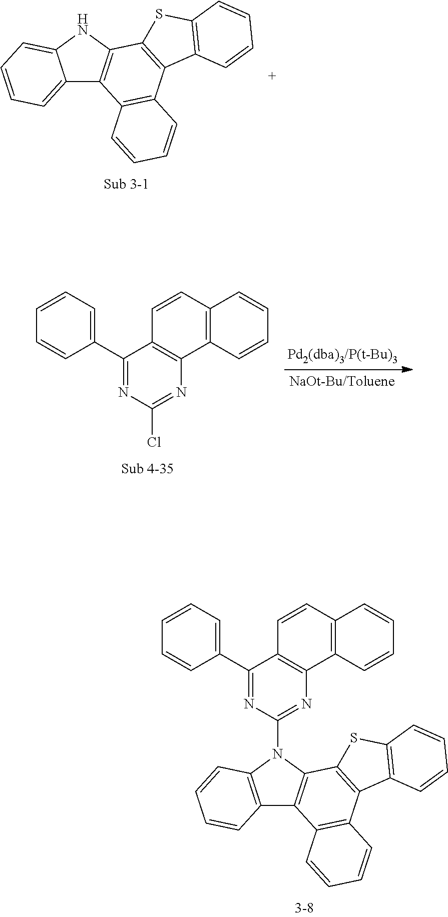 [SMILES: c1ccc2c(c1)[nH]c1c3sc4ccccc4c3c3ccccc3c21 ; Clc1nc(-c2ccccc2)c2ccc3ccccc3c2n1 ; c1ccc(-c2nc(-n3c4ccccc4c4c5ccccc5c5c6ccccc6sc5c43)nc3c2ccc2ccccc23)cc1]

Sub 3-1 (10 g, 30.92 mmol), toluene (325 mL), Sub 4-35 (9.89 g, 34.01 mmol), Pd₂(dba)₃ (0.85 g, 0.93 mmol), P(t-Bu)₃ (0.38 g, 1.86 mmol), NaOt-Bu (8.91 g, 92.76 mmol) were carried out in the same manner as in 3-1 to give the product. (13.04 g, 73%).

### Synthesis Example of 3-11

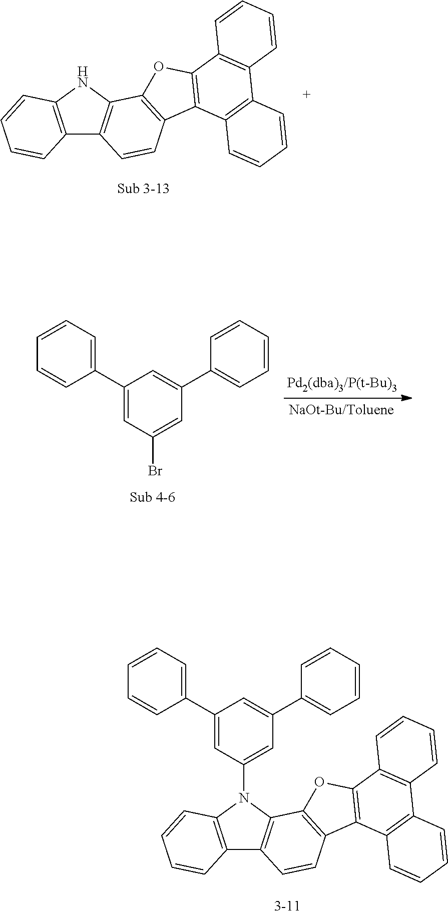 [SMILES: c1ccc2c(c1)[nH]c1c2ccc2c1oc1c3ccccc3c3ccccc3c21 ; Brc1cc(-c2ccccc2)cc(-c2ccccc2)c1 ; c1ccc(-c2cc(-c3ccccc3)cc(-n3c4ccccc4c4ccc5c(oc6c7ccccc7c7ccccc7c56)c43)c2)cc1]

Sub 3-13 (10 g, 27.98 mmol), toluene (294 ml), Sub 4-6 (9.52 g, 30.78 mmol), Pd₂(dba)₃ (0.77 g, 0.84 mmol), P(t-Bu)₃ (0.34 g, 1.68 mmol), NaOt-Bu (8.07 g, 83.94 mmol) were carried out in the same manner as in 3-1 to give the product. (12.45 g, 76%).

### Synthesis Example of 3-16

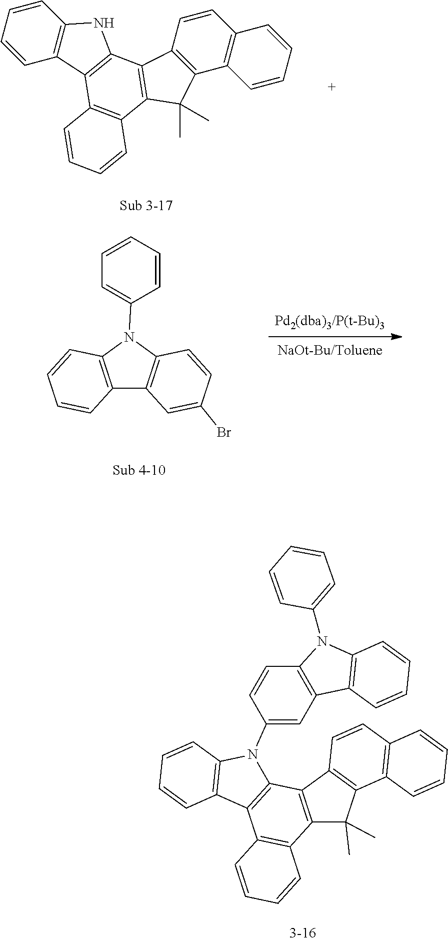 [SMILES: CC1(C)c2c(ccc3ccccc23)-c2c1c1ccccc1c1c2[nH]c2ccccc21 ; Brc1ccc2c(c1)c1ccccc1n2-c1ccccc1 ; CC1(C)c2c(ccc3ccccc23)-c2c1c1ccccc1c1c3ccccc3n(-c3ccc4c(c3)c3ccccc3n4-c3ccccc3)c21]

Sub 3-17 (10 g, 26.08 mmol), toluene (274 ml), Sub 4-10 (9.24 g, 28.68 mmol), Pd₂(dba)₃ (0.72 g, 0.78 mmol), P(t-Bu)₃ (0.32 g, 1.56 mmol), NaOt-Bu (7.52 g, 78.23 mmol) were carried out in the same manner as in 3-1 to give the product. (11.08 g, 68%).

### Synthesis Example of 3-17

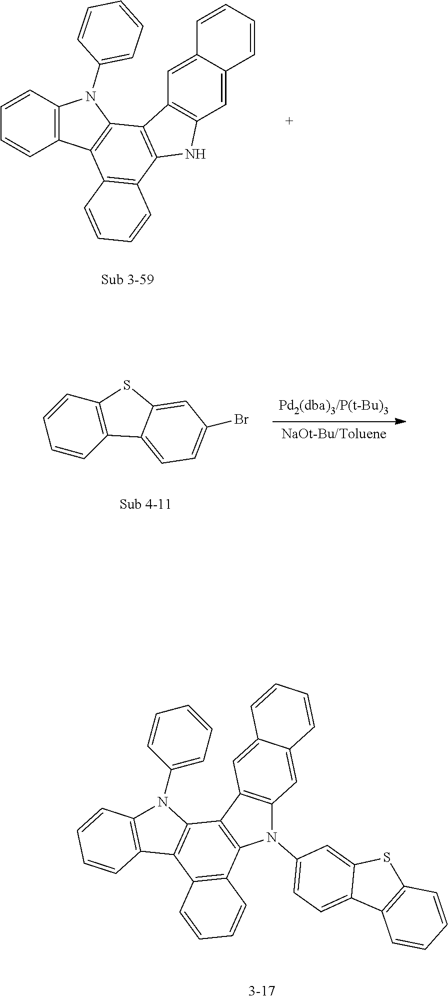 [SMILES: c1ccc(-n2c3ccccc3c3c4ccccc4c4[nH]c5cc6ccccc6cc5c4c32)cc1 ; Brc1ccc2c(c1)sc1ccccc12 ; c1ccc(-n2c3ccccc3c3c4ccccc4c4c(c5cc6ccccc6cc5n4-c4ccc5c(c4)sc4ccccc45)c32)cc1]

Sub 3-59 (10 g, 23.12 mmol), toluene (243 ml), Sub 4-11 (6.69 g, 25.43 mmol), Pd₂(dba)₃ (0.64 g, 0.69 mmol), P(t-Bu)₃ (0.28 g, 1.39 mmol), NaOt-Bu (6.67 g, 69.36 mmol) were carried out in the same manner as in 3-1 to give the product. (11.8 g, 83%).

### Synthesis Example of 3-47

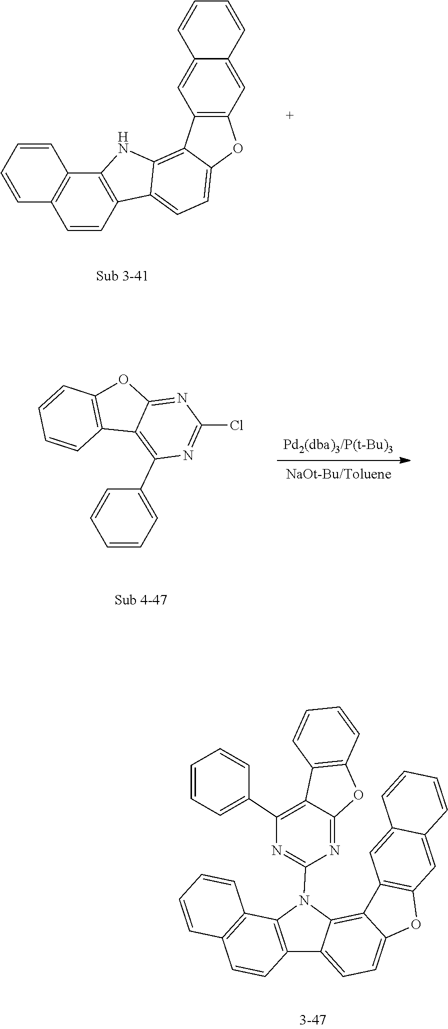 [SMILES: c1ccc2cc3c(cc2c1)oc1ccc2c4ccc5ccccc5c4[nH]c2c13 ; Clc1nc(-c2ccccc2)c2c(n1)oc1ccccc12 ; c1ccc(-c2nc(-n3c4c5ccccc5ccc4c4ccc5oc6cc7ccccc7cc6c5c43)nc3oc4ccccc4c23)cc1]

Sub 3-41 (10 g, 27.98 mmol), toluene (294 ml), Sub 4-47 (8.64 g, 30.78 mmol), Pd₂(dba)₃ (0.77 g, 0.84 mmol), P(t-Bu)₃ (0.34 g, 1.68 mmol), NaOt-Bu (8.07 g, 83.94 mmol) were carried out in the same manner as in 3-1 to give the product. (11.45 g, 68%).

### Synthesis Example of 3-52

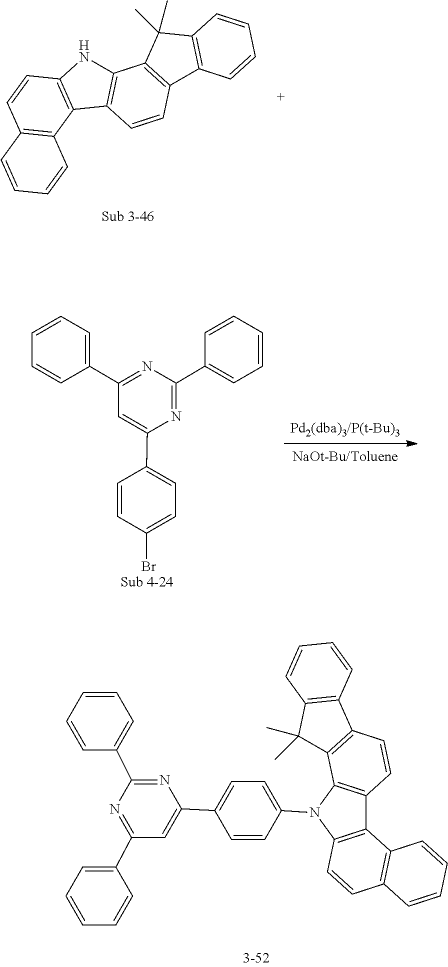 [SMILES: CC1(C)c2ccccc2-c2ccc3c([nH]c4ccc5ccccc5c43)c21 ; Brc1ccc(-c2cc(-c3ccccc3)nc(-c3ccccc3)n2)cc1 ; CC1(C)c2ccccc2-c2ccc3c4c5ccccc5ccc4n(-c4ccc(-c5cc(-c6ccccc6)nc(-c6ccccc6)n5)cc4)c3c21]

Sub 3-46 (10 g, 29.99 mmol), toluene (500 mL), Sub 4-24 (12.78 g, 32.99 mmol), Pd₂(dba)₃ (0.82 g, 0.90 mmol), P(t-Bu)₃ (0.36 g, 1.80 mmol), NaOt-Bu (8.65 g, 89.97 mmol) were carried out in the same manner as in 3-1 to give the product. (13.82 g, 72%).

### Synthesis Example of 3-70

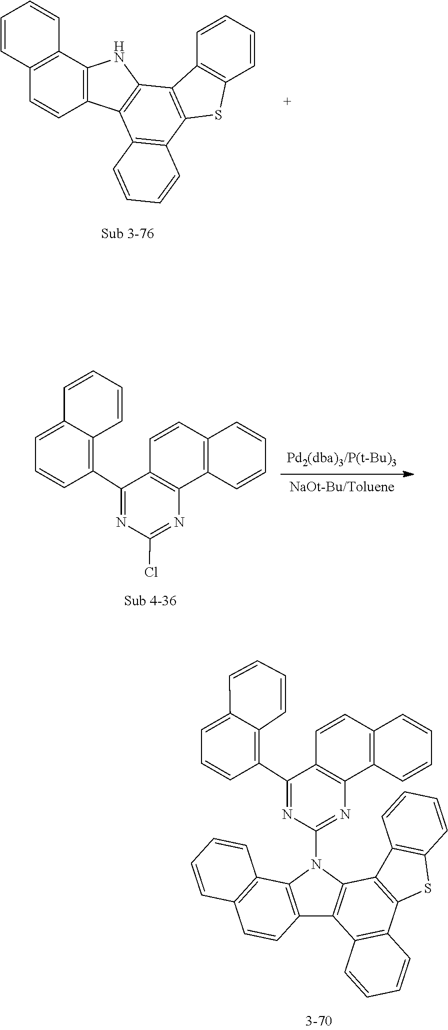 [SMILES: c1ccc2c(c1)ccc1c2[nH]c2c1c1ccccc1c1sc3ccccc3c21 ; Clc1nc(-c2cccc3ccccc23)c2ccc3ccccc3c2n1 ; c1ccc2c(-c3nc(-n4c5c6ccccc6ccc5c5c6ccccc6c6sc7ccccc7c6c54)nc4c3ccc3ccccc34)cccc2c1]

Sub 3-76 (10 g, 26.78 mmol), toluene (281 ml), Sub 4-36 (10.04 g, 29.45 mmol), Pd₂(dba)₃ (0.74 g, 0.80 mmol), P(t-Bu)₃ (0.33 g, 1.61 mmol), NaOt-Bu (7.72 g, 80.33 mmol) were carried out in the same manner as in 3-1 to give the product. (12.16 g, 67%).

### Synthesis Example of 3-92

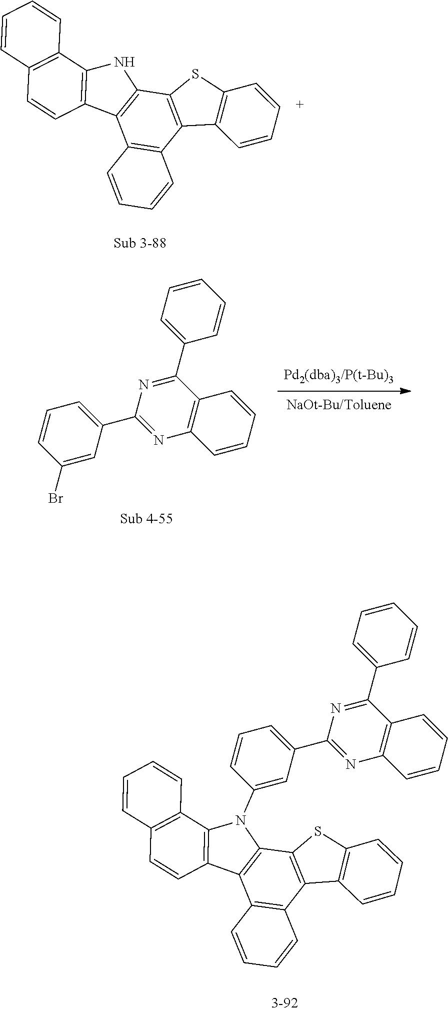 [SMILES: c1ccc2c(c1)ccc1c2[nH]c2c3sc4ccccc4c3c3ccccc3c12 ; Brc1cccc(-c2nc(-c3ccccc3)c3ccccc3n2)c1 ; c1ccc(-c2nc(-c3cccc(-n4c5c6ccccc6ccc5c5c6ccccc6c6c7ccccc7sc6c54)c3)nc3ccccc23)cc1]

Sub 3-88 (10 g, 26.78 mmol), toluene (281 ml), Sub 4-55 (9.67 g, 26.78 mmol), Pd₂(dba)₃ (0.37 g, 0.40 mmol), P(t-Bu)₃ (0.16 g, 0.80 mmol), NaOt-Bu (3.86 g, 40.16 mmol) were carried out in the same manner as in 3-1 to give the product. (12.25 g, 70%).

TABLE 6

| compound | FD-MS | compound | FD-MS |
|---|---|---|---|
| 3-1 | m/z = 458.18(C₃₄H₂₂N₂ = 458.56) | 3-2 | m/z = 449.12(C₃₂H₁₉NS = 449.57) |
| 3-3 | m/z = 433.15(C₃₂H₁₉NO = 433.51) | 3-4 | m/z = 535.23(C₄₁H₂₉N = 535.69) |
| 3-5 | m/z = 399.11(C₂₈H₁₇NS = 399.51) | 3-6 | m/z = 527.15(C₃₆H₂₁N₃S = 527.65) |
| 3-7 | m/z = 583.12(C₃₈H₂₁N₃S₂ = 583.73) | 3-8 | m/z = 577.16(C₄₀H₂₃N₃S = 577.71) |
| 3-9 | m/z = 627.18(C₄₄H₂₅N₃S = 627.77) | 3-10 | m/z = 475.14(C₃₄H₂₁NS = 475.61) |
| 3-11 | m/z = 585.21(C₄₄H₂₇NO = 585.71) | 3-12 | m/z = 509.21(C₃₉H₂₇N = 509.65) |
| 3-13 | m/z = 509.19(C₃₇H₂₃N₃ = 509.61) | 3-14 | m/z = 451.11(C₃₀H₁₇N₃S = 451.55) |
| 3-15 | m/z = 588.20(C₄₁H₂₄N₄O = 588.67) | 3-16 | m/z = 624.26(C₄₇H₃₂N₂ = 624.79) |
| 3-17 | m/z = 614.18(C₄₄H₂₆N₂S = 614.77) | 3-18 | m/z = 449.12(C₃₂H₁₉NS = 449.57) |
| 3-19 | m/z = 573.17(C₄₂H₂₃NO₂ = 573.65) | 3-20 | m/z = 664.26(C₄₈H₃₂N₄ = 664.81) |
| 3-21 | m/z = 624.26(C₄₇H₃₂N₂ = 624.79) | 3-22 | m/z = 603.18(C₄₂H₂₅N₃S = 603.74) |
| 3-23 | m/z = 664.23 (C₄₇H₂₈N₄O = 664.77) | 3-24 | m/z = 737.28(C₅₅H₃₅N₃ = 737.91) |
| 3-25 | m/z = 738.28(C₅₄H₃₄N₄ = 738.89) | 3-26 | m/z = 679.21(C₄₈H₂₉N₃S = 679.84) |
| 3-27 | m/z = 625.22(C₄₅H₂₇N₃O = 625.73) | 3-28 | m/z = 575.24(C₄₂H₂₉N₃ = 575.72) |
| 3-29 | m/z = 508.19(C₃₈H₂₄N₂ = 508.62) | 3-30 | m/z = 449.12(C₃₂H₁₉NS = 449.57) |
| 3-31 | m/z = 433.15(C₃₂H₁₉NO = 433.51) | 3-32 | m/z = 531.20(C₄₁H₂₅N = 531.66) |
| 3-33 | m/z = 608.23(C₄₆H₂₈N₂ = 608.74) | 3-34 | m/z = 475.14(C₃₄H₂₁NS = 475.61) |
| 3-35 | m/z = 384.13(C₂₇H₁₆N₂O = 384.44) | 3-36 | m/z = 614.25(C₄₄H₃₀N₄ = 614.75) |
| 3-37 | m/z = 508.19(C₃₈H₂₄N₂ = 508.62) | 3-38 | m/z = 449.12(C₃₂H₁₉NS = 449.57) |
| 3-39 | m/z = 433.15(C₃₂H₁₉NO = 433.51) | 3-40 | m/z = 459.20(C₃₅H₂₅N = 459.59) |
| 3-41 | m/z = 663.24(C₄₇H₂₉N₅ = 663.78) | 3-42 | m/z = 603.18(C₄₂H₂₅N₃S = 603.74) |
| 3-43 | m/z = 587.20(C₄₂H₂₅N₃O = 587.68) | 3-44 | m/z = 613.25(C₄₅H₃₁N₃ = 613.76) |
| 3-45 | m/z = 662.25(C₄₈H₃₀N₄ = 662.80) | 3-46 | m/z = 577.16(C₄₀H₂₃N₃S = 577.71) |
| 3-47 | m/z = 601.18(C₄₂H₂₃N₃O₂ = 601.67) | 3-48 | m/z = 759.27(C₅₇H₃₃N₃ = 759.91) |
| 3-49 | m/z = 589.22(C₄₂H₂₆N₄ = 586.70) | 3-50 | m/z = 630.19(C₄₃H₂₆N₄S = 630.77) |
| 3-51 | m/z = 613.22(C₄₄H₂₇N₃O = 613.72) | 3-52 | m/z = 639.27(C₄₇H₃₃N₃ = 639.80) |
| 3-53 | m/z = 508.19(C₃₈H₂₄N₂ = 508.62) | 3-54 | m/z = 449.12(C₃₂H₁₉NS = 449.57) |
| 3-55 | m/z = 433.15(C₃₂H₁₉NO = 433.51) | 3-56 | m/z = 609.25(C₄₇H₃₁N = 609.77) |
| 3-57 | m/z = 663.24(C₄₇H₂₉N₅ = 663.78) | 3-58 | m/z = 604.17(C₄₁H₂₄N₄S = 604.73) |
| 3-59 | m/z = 587.20(C₄₂H₂₅N₃O = 587.68) | 3-60 | m/z = 613.25(C₄₅H₃₁N₃ = 613.76) |
| 3-61 | m/z = 527.15(C₃₆H₂₁N₃S = 527.65) | 3-62 | m/z = 603.18(C₄₂H₂₅N₃S = 603.74) |
| 3-63 | m/z = 516.20(C₃₆H₁₆D₅N₃O = 516.61) | 3-64 | m/z = 605.23(C₄₃H₂₈FN₃ = 587.73) |
| 3-65 | m/z = 692.20(C₄₈H₂₈N₄S = 692.84) | 3-66 | m/z = 577.16(C₄₀H₂₃N₃S = 577.71) |
| 3-67 | m/z = 561.18(C₄₀H₂₃N₃O = 561.64) | 3-68 | m/z = 653.19(C₄₆H₂₇N₃S = 653.80) |
| 3-69 | m/z = 736.26(C₅₄H₃₂N₄ = 736.88) | 3-70 | m/z = 677.19(C₄₈H₂₇N₃S = 677.83) |
| 3-71 | m/z = 692.26(C₅₀H₂₄D₅N₃O = 692.83) | 3-72 | m/z = 743.24(C₅₃H₃₃N₃S = 743.93) |
| 3-73 | m/z = 703.21(C₅₀H₂₉N₃S = 703.86) | 3-74 | m/z = 603.18(C₄₂H₂₅N₃S = 603.74) |
| 3-75 | m/z = 659.15(C₄₄H₂₅N₃S₂ = 659.83) | 3-76 | m/z = 759.18(C₅₂H₂₉N₃S₂ = 759.95) |
| 3-77 | m/z = 475.14(C₃₄H₂₁NS = 475.61) | 3-78 | m/z = 616.20(C₄₄H₂₈N₂S = 616.78) |
| 3-79 | m/z = 728.15(C₄₇H₂₅FN₄S₂ = 728.86) | 3-80 | m/z = 818.25(C₅₈H₃₄N₄S = 819.00) |
| 3-81 | m/z = 628.17(C₄₃H₂₄N₄S = 628.75) | 3-82 | m/z = 809.20(C₅₆H₃₁N₃S₂ = 810.01) |
| 3-83 | m/z = 659.15(C₄₄H₂₅N₃S₂ = 659.83) | 3-84 | m/z = 723.27(C₅₁H₃₇N₃S = 629.72) |
| 3-85 | m/z = 844.27(C₆₀H₃₆N₄S = 845.04) | 3-86 | m/z = 667.17(C₄₆H₂₅N₃OS = 667.79) |
| 3-87 | m/z = 703.23(C₅₀H₂₉N₃O = 703.80) | 3-88 | m/z = 759.25(C₅₃H₃₃N₃O₃ = 759.87) |
| 3-89 | m/z = 677.19(C₄₈H₂₇N₃S = 677.83) | 3-90 | m/z = 683.15(C₄₆H₂₅N₃S₂ = 683.85) |
| 3-91 | m/z = 668.17(C₄₅H₂₄N₄OS = 668.77) | 3-92 | m/z = 653.80(C₄₆H₂₇N₃S = 653.80) |
| 3-93 | m/z = 604.17(C₄₁H₂₄N₄S = 604.73) | 3-94 | m/z = 650.27(C₄₉H₃₄N₂ = 650.83) |
| 3-95 | m/z = 878.30(C₆₄H₃₈N₄O = 879.04) | 3-96 | m/z = 775.26(C₅₇H₃₃N₃O = 775.91) |
| 3-97 | m/z = 588.20(C₄₁H₂₄N₄O = 588.67) | 3-98 | m/z = 561.18(C₄₀H₂₃N₃O = 561.64) |
| 3-99 | m/z = 601.18(C₄₂H₂₃N₃O₂ = 601.67) | 3-100 | m/z = 664.23 (C₄₇H₂₈N₄O = 664.77) |

Otherwise, the synthesis examples of the present invention represented by Formulas (1) and (2) have been described, but these are all based on the Buchwald-Hartwig cross coupling reaction, Suzuki cross-coupling reaction, Intramolecular acid-induced cyclization reaction (J. mater. Chem.1999, 9, 2095), Pd(II)-catalyzed oxidative cyclization reaction (Org. Lett.2011, 13, 5504), Grignard reaction, Cyclic Dehydration reaction and PPh₃-mediated reductive cyclization reaction (J. Org. Chem. 2005, 70, 5014), and those skilled in the art will readily understand that the above reaction proceeds even when, besides the substituent specified in the specific synthesis example, other substituents (Substituents such as Ar¹ to Ar⁶, L¹ to L⁶, R¹ to R⁵, X¹, X², A and B) defined in Formula (1) and Formula (2) are bonded.

### Evaluation of Manufacture of Organic Electric Element

### Example 1) Manufacture and Evaluation of Red Organic Light Emitting Diode

### (Emitting Layer Mixed Phosphorescent Host)

First, on an ITO layer (anode) formed on a glass substrate, N¹-(naphthalen-2-yl)-N⁴,N⁴-bis(4-(naphthalen-2-yl(phenyl)amino)phenyl)-N¹-phenyl benzene-1,4-diamine (hereinafter will be abbreviated as 2-TNATA) was vacuum-deposited to form a hole injection layer with a thickness of 60 nm, and N,N′-bis(1-naphthalenyl)-N,N′-bis-phenyl-(1,1′-biphenyl)-4,4′-diamine (hereinafter will be abbreviated as NPB) was vacuum-deposited to form a hole transport layer with a thickness of 60 nm. On the hole transport layer, a mixture of the compounds represented by Formulas (1) and (2) as a host in a ratio of 3:7 was used as a host, and as a dopant, an emitting layer with a thickness of 30 nm was deposited on the hole transport layer by doping (piq)₂Ir(acac) [bis-(1-phenylisoquinolyl)iridium(III)acetylacetonate] with a weight of 5%. (1,1′-bisphenyl)-4-olato)bis(2-methyl-8-quinolinolato)aluminum (hereinafter abbreviated as BAlq) was vacuum deposited as a hole blocking layer to a thickness of 10 nm, and tris(8-quinolinol)aluminum (hereinafter abbreviated as Alq3) was deposited to a thickness of 40 nm as an electron transport layer. After that, an alkali metal halide, LiF was vacuum deposited as an electron injection layer to a thickness of 0.2 nm, and Al was deposited to a thickness of 150 nm to form a cathode to manufacture an OLED.

### [Example 2] to [Example 61] Red Organic Light Emitting Diode

### (Emitting Layer Mixed Phosphorescent Host)

The inventive compound represented by Formula (1) and Formula (2) of the present invention as the host material of the emitting layer was prepared in the same manner as in Example 1 to prepare an organic electroluminescent device, except for using the compounds of the present invention described in Table 7 below,

### Comparative Examples 1 to 3

An organic electroluminescent device was manufactured in the same manner as in Example 1, except that the compound represented by Formula (2) was used as a host alone.

### Comparative Example 4

An organic electroluminescent device was manufactured in the same manner as in Example 1, except that the comparative compound 1 was used as a host alone.

### Comparative Example 5

An organic electroluminescent device was manufactured in the same manner as in Example 1, except that the comparative compound 2 was used as a host alone.

### Comparative Example 6

An organic electroluminescent device was manufactured in the same manner as in Example 1, except that the comparative compound 3 was used as a host alone.

### Comparative Example 7

An organic electroluminescent device was manufactured in the same manner as in Example 1, except that the comparative compound 4 was used as a host alone.

### Comparative Example 8

An organic electroluminescent device was manufactured in the same manner as in Example 1, except that comparative compound 1 and 2 were mixed and used as a host.

### Comparative Example 9

An organic electroluminescent device was manufactured in the same manner as in Example 1, except that comparative compound 3 and 4 were mixed and used as a host.

To the OLEDs which were manufactured by example 1 to 61 and comparative examples 1 to 9, a forward bias direct current voltage was applied, and electroluminescent (EL) properties were measured using PR-650 of Photoresearch Co., and T95 life was measured using a life measuring apparatus manufactured by McScience Inc. with a reference luminance of 2500 cd/m². The measurement results are shown in Tables 7 and 8 below.

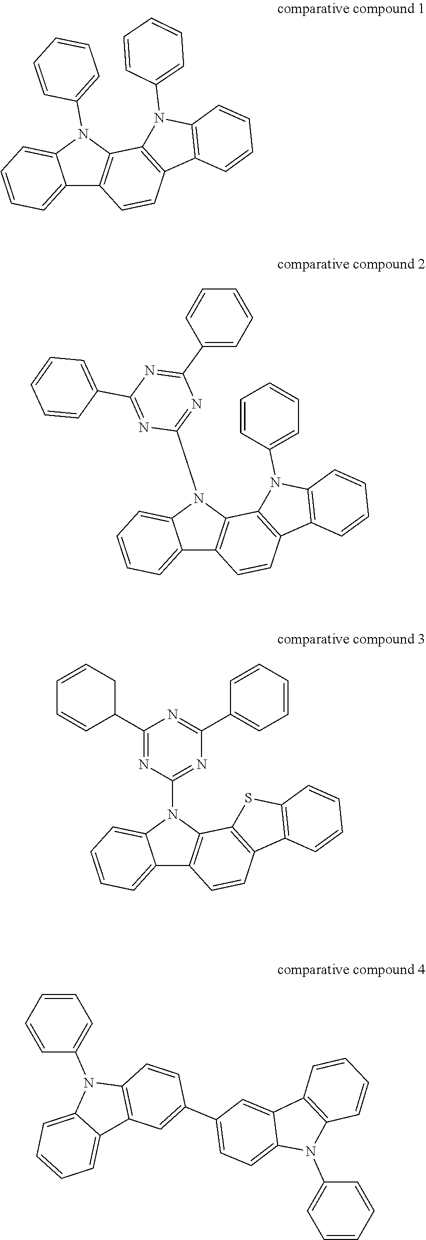 [SMILES: c1ccc(-n2c3ccccc3c3ccc4c5ccccc5n(-c5ccccc5)c4c32)cc1 ; c1ccc(-c2nc(-c3ccccc3)nc(-n3c4ccccc4c4ccc5c6ccccc6n(-c6ccccc6)c5c43)n2)cc1 ; C1=CCC(c2nc(-c3ccccc3)nc(-n3c4ccccc4c4ccc5c6ccccc6sc5c43)n2)C=C1 ; c1ccc(-n2c3ccccc3c3cc(-c4ccc5c(c4)c4ccccc4n5-c4ccccc4)ccc32)cc1]

When the second host is fixed and various first hosts are mixed.

TABLE 7

| | | | | Current | Brightness | | Lifetime |
|---|---|---|---|---|---|---|---|
| | First host | Second host | Voltage | Density | (cd/m²) | Efficiency | T(95) |
| comparative | — | compound(3-6) | 6.1 | 15.7 | 2500 | 15.9 | 108.9 |
| example(1) | | | | | | | |
| comparative | — | compound(3-61) | 6.3 | 16.2 | 2500 | 15.5 | 104.2 |
| example(2) | | | | | | | |
| comparative | — | compound(3-74) | 6.4 | 16.2 | 2500 | 15.4 | 103.2 |
| example(3) | | | | | | | |
| comparative | — | comparative | 6.9 | 18.6 | 2500 | 13.4 | 84.3 |
| example(4) | | compound 1 | | | | | |
| comparative | — | comparative | 6.8 | 18.3 | 2500 | 13.7 | 83.3 |
| example(5) | | compound 2 | | | | | |
| comparative | — | comparative | 6.7 | 17.5 | 2500 | 14.3 | 87.4 |
| example(6) | | compound 3 | | | | | |
| comparative | — | comparative | 6.9 | 18.4 | 2500 | 13.6 | 82.9 |
| example(7) | | compound 4 | | | | | |
| comparative | comparative | comparative | 5.9 | 13 | 2500 | 19.3 | 103.8 |
| example(8) | compound 1 | compound 2 | | | | | |
| comparative | comparative | comparative | 5.6 | 10.5 | 2500 | 23.9 | 108.9 |
| example(9) | compound 3 | compound 4 | | | | | |
| example(1) | compound 1-3 | compound 3-6 | 4.3 | 6.2 | 2500 | 40.5 | 142.4 |
| example(2) | compound 1-5 | compound 3-6 | 4.6 | 8.0 | 2500 | 31.1 | 128.2 |
| example(3) | compound 1-10 | compound 3-6 | 4.5 | 7.1 | 2500 | 35.2 | 137.8 |
| example(4) | compound 1-15 | compound 3-6 | 4.4 | 8.2 | 2500 | 30.5 | 131.4 |
| example(5) | compound 1-16 | compound 3-6 | 4.5 | 7.4 | 2500 | 33.6 | 135.5 |
| example(6) | compound 1-19 | compound 3-6 | 4.5 | 7.6 | 2500 | 32.8 | 133.9 |
| example(7) | compound 2-1 | compound 3-6 | 4.4 | 5.9 | 2500 | 42.3 | 137.8 |
| example(8) | compound 2-5 | compound 3-6 | 4.4 | 6.2 | 2500 | 40.6 | 134.5 |
| example(9) | compound 2-6 | compound 3-6 | 4.6 | 8.1 | 2500 | 30.7 | 129.1 |
| example(10) | compound 2-10 | compound 3-6 | 4.6 | 8.4 | 2500 | 29.8 | 127.4 |
| example(11) | compound 2-14 | compound 3-6 | 4.5 | 7.1 | 2500 | 35.2 | 133.9 |
| example(12) | compound 2-15 | compound 3-6 | 4.5 | 7.2 | 2500 | 34.8 | 130.5 |
| example(13) | compound 2-18 | compound 3-6 | 4.6 | 7.5 | 2500 | 33.5 | 129.2 |
| example(14) | compound 2-26 | compound 3-6 | 4.5 | 6.7 | 2500 | 37.2 | 132.4 |
| example(15) | compound 2-36 | compound 3-6 | 4.5 | 6.9 | 2500 | 36.3 | 132.7 |
| example(16) | compound 2-63 | compound 3-6 | 4.6 | 7.9 | 2500 | 31.6 | 128.8 |
| example(17) | compound 2-65 | compound 3-6 | 4.6 | 8.3 | 2500 | 30.3 | 127.5 |
| example(17) | compound 2-76 | compound 3-6 | 4.4 | 6.2 | 2500 | 40.1 | 144.5 |

When the first host material and various second host materials are mixed.

TABLE 8

| | | | | Current | Brightness | | Lifetime |
|---|---|---|---|---|---|---|---|
| | First host | Second host | Voltage | Density | (cd/m²) | Efficiency | T(95) |
| example(18) | compound 1-3 | compound 3-7 | 4.4 | 7.2 | 2500 | 34.6 | 134.4 |
| example(19) | | compound 3-8 | 4.4 | 7.2 | 2500 | 34.7 | 132.7 |
| example(20) | | compound 3-9 | 4.4 | 7.1 | 2500 | 35.1 | 132.1 |
| example(21) | | compound 3-15 | 4.4 | 7.8 | 2500 | 32.0 | 130.3 |
| example(22) | | compound 3-37 | 4.5 | 8.6 | 2500 | 29.1 | 128.5 |
| example(23) | | compound 3-46 | 4.4 | 7.1 | 2500 | 35.1 | 132.1 |
| example(24) | | compound 3-50 | 4.5 | 7.7 | 2500 | 32.4 | 129.5 |
| example(25) | | compound 3-61 | 4.3 | 6.6 | 2500 | 37.9 | 139.2 |
| example(26) | | compound 3-74 | 4.4 | 7.1 | 2500 | 35.4 | 130.8 |
| example(27) | | compound 3-89 | 4.3 | 7.1 | 2500 | 35.3 | 132.8 |
| example(28) | | compound 3-90 | 4.3 | 7.1 | 2500 | 35.3 | 132.5 |
| example(29) | compound 1-10 | compound 3-7 | 4.6 | 8.0 | 2500 | 31.4 | 132.2 |
| example(30) | | compound 3-8 | 4.6 | 7.9 | 2500 | 31.6 | 131.7 |
| example(31) | | compound 3-9 | 4.6 | 8.6 | 2500 | 29.1 | 130.2 |
| example(32) | | compound 3-15 | 4.6 | 9.1 | 2500 | 27.4 | 127.1 |
| example(33) | | compound 3-37 | 4.7 | 9.6 | 2500 | 26.2 | 126.4 |
| example(34) | | compound 3-46 | 4.6 | 8.6 | 2500 | 29.0 | 130.1 |
| example(35) | | compound 3-50 | 4.6 | 9.3 | 2500 | 26.8 | 126.5 |
| example(36) | | compound 3-61 | 4.5 | 7.5 | 2500 | 33.3 | 134.5 |
| example(37) | | compound 3-74 | 4.6 | 8.0 | 2500 | 31.1 | 131.8 |
| example(38) | | compound 3-89 | 4.4 | 8.4 | 2500 | 29.9 | 129.2 |
| example(39) | | compound 3-90 | 4.4 | 8.5 | 2500 | 29.4 | 130.2 |
| example(40) | compound 2-1 | compound 3-7 | 4.5 | 7.3 | 2500 | 34.0 | 128.0 |
| example(41) | | compound 3-8 | 4.5 | 7.5 | 2500 | 33.3 | 128.3 |
| example(42) | | compound 3-9 | 4.5 | 7.4 | 2500 | 33.8 | 128.9 |
| example(43) | | compound 3-15 | 4.5 | 8.3 | 2500 | 30.1 | 126.0 |
| example(44) | | compound 3-37 | 4.6 | 9.9 | 2500 | 25.1 | 123.9 |
| example(45) | | compound 3-46 | 4.5 | 8.0 | 2500 | 31.2 | 128.1 |
| example(46) | | compound 3-50 | 4.6 | 8.2 | 2500 | 30.4 | 126.9 |
| example(47) | | compound 3-61 | 4.5 | 6.2 | 2500 | 40.5 | 133.8 |
| example(48) | | compound 3-74 | 4.5 | 7.1 | 2500 | 35.4 | 130.6 |
| example(49) | | compound 3-89 | 4.4 | 8.1 | 2500 | 30.9 | 126.2 |
| example(50) | | compound 3-90 | 4.4 | 8.1 | 2500 | 30.7 | 125.2 |
| example(51) | compound 2-14 | compound 3-7 | 4.5 | 7.9 | 2500 | 31.6 | 127.9 |
| example(52) | | compound 3-8 | 4.5 | 7.9 | 2500 | 31.5 | 127.9 |
| example(53) | | compound 3-9 | 4.5 | 8.5 | 2500 | 29.6 | 128.3 |
| example(54) | | compound 3-15 | 4.6 | 9.2 | 2500 | 27.2 | 128.1 |
| example(55) | | compound 3-37 | 4.7 | 9.3 | 2500 | 26.9 | 125.8 |
| example(56) | | compound 3-46 | 4.5 | 8.4 | 2500 | 29.9 | 128.8 |
| example(57) | | compound 3-50 | 4.6 | 9.0 | 2500 | 27.7 | 128.4 |
| example(58) | | compound 3-61 | 4.5 | 7.1 | 2500 | 35.2 | 133.9 |
| example(59) | | compound 3-74 | 4.5 | 8.2 | 2500 | 30.6 | 126.4 |
| example(60) | | compound 3-89 | 4.4 | 8.1 | 2500 | 30.8 | 129.0 |
| example(61) | | compound 3-90 | 4.4 | 8.5 | 2500 | 29.4 | 128.2 |
| example(61′) | compound 2-76 | compound 3-101 | 4.3 | 5.9 | 2500 | 42.5 | 143.8 |

As can be seen from the results of Table 7 and 8, when the organic electric element material of the present invention represented by Formulas (1) and (2) is mixed and used as a phosphorescent host (Examples 1 to 61), it was confirmed that the driving voltage, efficiency, and life span were significantly improved as compared with the element using a single material (comparative examples 1 to 7).

More specifically, in Comparative Examples 1 to 7, wherein the compounds of the present invention represented by Formula (2) and comparative compounds 1 to 4 are used alone as a phosphorescent host, Comparative Examples 1 to 3 using the compounds (3-6, 3-61, and 3-74) of the present invention had higher efficiency and longer life span than Comparative Examples 4 to 7 using the comparative compound.

Also, Comparative Example 8 and 9 wherein Comparative Compound 1 and 2 or Comparative Compound 3 and 4 were mixed and used as a phosphorescent host were found to exhibit higher efficiency than Comparative Examples 1 to 7 using the single substance.

Comparing Comparative Example 8 with 9, Comparative Example 9 using a mixture containing a polycyclic compound having a different heteroatom (N, S) among the 5-membered compounds had higher efficiency than Comparative Example 8 mixed a 5-membered heterocyclic compound having the same nitrogen atom.

And it was confirmed that Example 1 to 61 using the mixture of the compound of Formula (1) and (2) as a host exhibited remarkably high efficiency and long life span than the Comparative Example 1 to 9.

On the basis of the above experimental results, the inventors of the present invention have found that, in the case of a mixture of the substance of Formulas (1) and (2), they have novel characteristics other than those for the respective materials, and have measured the PL lifetime using the substance of Formula (1), the substance of Formula (2), and the mixture of the present invention. As a result, it was confirmed that a new PL wavelength was formed when the compounds of Formulas (1) and (2) were mixed, and the decreasing and disappearing time of the newly formed PL wavelength increased from about 60 times to about 360 times compared to the reduction and disappearance times of substances Formula (1) and (2), respectively. It is considered when mixed with the compound of the present invention, not only electrons and holes are moved through the energy level of each substance, but also the efficiency and life span are increased by electron, hole transport or energy transfer by a new region (exciplex) having a new energy level formed due to mixing. As a result, when the mixture of the present invention is used, the mixed thin film is an important example showing exciplex energy transfer and light emitting process.

The reason why the combination of the present invention is superior to Comparative Examples 8 to 9 in which a comparative compound is used as a phosphorescent host is that the high T1 and high LUMO energy values improve the electron blocking ability and allow more holes to be moved to the emitting layer more quickly and easily when a compound represented by Formula (1) having a strong hole property is mixed with a polycyclic compound represented by Formula (2), which is characterized not only by electron but also by hole stability and high T1. As a result, the charge balance in the emitting layer of holes and electrons is increased, so that light emission is well performed inside the emitting layer rather than at the interface of the hole transport layer, and therefore the deterioration in the HTL interface is also reduced, thereby maximizing the driving voltage, efficiency and life span of the device. Among the compounds represented by Formula (1), 1) when AO and Ar² are of the type in which the ring is curled, it has been confirmed that compounds having at least one of Ar³ and Ar⁴ substituted with biphenyl exhibits the best results in terms of the driving voltage, the efficiency and the lifetime, and compounds having at least one of Ar³ and Ar⁴ substituted with Dibenzothiophen or Dibenzofuran were found to be excellent in efficiency and lifetime, and in the case of compounds in which at least one of Ar³ and Ar⁴ is substituted with fluorene, the driving voltage is excellent. 2) When Ar¹ and Ar² do not form a ring, compounds in which both Ar³ and Ar⁴ were substituted with naphthyl showed the best results in terms of driving voltage, efficiency, and lifetime, and compounds having at least one of Ar³ and Ar⁴ substituted with Dibenzothiophen or Dibenzofuran were confirmed to have excellent efficiency and lifetime. That is, it is concluded that the combination of Formula (1) and Formula (2) is electrochemically synergistic to improve the performance of the device as a whole.

Also, Table 8 shows the results obtained using Table 7, in which a first host with high performance was fixed and a variety of second hosts were mixed. As a result, as the first host, when compounds 1-3, 1-10, 2-1 and 2-14 having the best driving voltage, efficiency, and lifetime and, as the second host, the compounds 3-7, 3-8, 3-9, 3-15, 3-37, 3-46, 3-50, 3-61, 3-74, 3-89 and 3-90 were mixed, it can be seen that the driving voltage, efficiency and lifetime can be remarkably improved by using two mixed host materials as compared with the case of using a single host material.

### [Example 62] to [Example 69] Manufacture and Evaluation of Red Organic Light Emitting Diode by Mixing Ratio

An organic electroluminescent device was manufactured in the same manner as in Example 1, except that the materials are used in different mixing ratios as listed in Table 9.

TABLE 9

| | | | Mixing | | | | | |
|---|---|---|---|---|---|---|---|---|
| | | | ratio(fist | | | | | |
| | | Second | host:second | | Current | Brightness | | Lifetime |
| | First host | host | host) | Voltage | Density | (cd/m²) | Efficiency | T(95) |
| example(62) | compound | compound | 2:8 | 4.3 | 6.1 | 2500 | 41.2 | 140.5 |
| example(63) | 1-3 | 3-6 | 3:7 | 4.3 | 6.2 | 2500 | 40.5 | 142.4 |
| example(64) | | | 4:6 | 4.6 | 6.7 | 2500 | 37.1 | 136.2 |
| example(65) | | | 5:5 | 4.7 | 7.8 | 2500 | 31.9 | 127.5 |
| example(66) | compound | compound | 2:8 | 4.4 | 6.4 | 2500 | 39.1 | 136.4 |
| example(67) | 2-1 | 3-61 | 3:7 | 4.5 | 6.2 | 2500 | 40.5 | 133.8 |
| example(68) | | | 4:6 | 4.6 | 7.0 | 2500 | 35.6 | 130.5 |
| example(69) | | | 5:5 | 4.8 | 8.0 | 2500 | 31.3 | 124.6 |

As shown in Table 9, the mixture of the compound of the present invention was measured by fabricating the device in (2:8, 3:7, 4:6, 5:5). To explain the results in detail, in the result of the mixture of the compound 1-3 and 3-6, the results of the driving voltage, the efficiency and the life span were similarly excellent at 2:8 and 3:7, but as the ratio of the first host increases, such as 4:6 and 5:5, the results of the driving voltage, the efficiency and the life span are gradually decreased, this was also the same in the result of the mixture of the compound 2-1 and 3-61. This can be explained by the fact that the charge balance in the emitting layer is maximized when an appropriate amount of the compound represented by Formula (1) having strong hole properties such as 2:8 and 3:7 is mixed.

### Example 70

### Red Organic Light Emitting Diode

### (Emitting Auxiliary Layer, Phosphorescent Host)

First, on an ITO layer (anode) formed on a glass substrate, N¹-(naphthalen-2-yl)-N⁴,N⁴-bis(4-(naphthalen-2-yl(phenyl)amino)phenyl)-N¹-phenyl benzene-1,4-diamine (hereinafter will be abbreviated as 2-TNATA) was vacuum-deposited to form a hole injection layer with a thickness of 60 nm, and N,N′-bis(1-naphthalenyl)-N,N′-bis-phenyl-(1,1′-biphenyl)-4,4′-diamine (hereinafter will be abbreviated as NPB) was vacuum-deposited to form a hole transport layer with a thickness of 60 nm. Subsequently, 4,4-bis[N-(1-naphthyl)-N-phenylamino]biphenyl (hereinafter abbreviated as —NPD) was vapor-deposited as a hole transport compound on the film to a thickness of 60 nm to form a hole transport layer. Subsequently, Compound 2-76 of the present invention was vacuum-deposited as an emitting auxiliary layer material to a thickness of 20 nm to form an emitting auxiliary layer. Subsequently, on the hole transport layer, the compound 3-6 of the present invention was doped as a host material and bis-(1-phenylisoquinolyl)iridium (III)acetylacetonate (hereinafter abbreviated as “(piq)₂Ir(acac)”) was doped as a dopant in a weight ratio of 95:5, followed by vacuum evaporation to a thickness of 30 nm to form an emitting layer. Subsequently, ((1,1′-biphenyl)-4-oleato)bis(2-methyl-8-quinolinolato)aluminum (hereinafter will be abbreviated as BAlq) was vacuum deposited on the emitting layer to a thickness of 5 nm to form a hole blocking layer, and Bis(10-hydroxybenzo[h]quinolinato)beryllium (hereinafter will be abbreviated as BeBq2) was vacuum deposited on the hole blocking layer to a thickness of 40 nm to form an electron transport layer. After that, on the electron transport layer, an alkali metal halide, LiF was vacuum deposited as an electron injection layer to a thickness of 0.2 nm, and Al was deposited to a thickness of 150 nm to form a cathode to manufacture an OLED.

### [Example 71] to [Example 85] Red Organic Light Emitting Diode (Emitting Auxiliary Layer, Phosphorescent Host)

An organic electroluminescent device was manufactured in the same manner as in Example 70, except that the compound represented by Formula (1) shown in Table 10 was used as the emitting auxiliary layer material, and the compound represented by Formula (2) shown in Table 10 of the present invention was used as the host material of the emitting layer.

To the OLEDs which were manufactured by examples and comparative examples, a forward bias direct current voltage was applied, and electroluminescent (EL) properties were measured using PR-650 of Photoresearch Co., and T95 life was measured using a life measuring apparatus manufactured by McScience Inc. with a reference luminance of 2500 cd/m². In the following table, the manufacture of a device and the results of evaluation are shown.

### Comparative 10

An organic electroluminescent device was manufactured in the same manner as in Example 70, except that the emitting auxiliary layer was not used and Comparative Compound 5 was used as a host.

### Comparative Compound 5

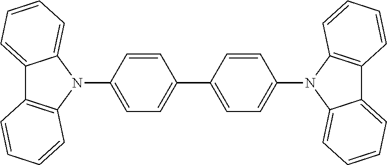 [SMILES: c1ccc2c(c1)c1ccccc1n2-c1ccc(-c2ccc(-n3c4ccccc4c4ccccc43)cc2)cc1]

### Comparative 11-14

An organic electroluminescent device was manufactured in the same manner as in Example 70, except that the emitting auxiliary layer was not used.

TABLE 10

| | Emitting | | | | | | |
|---|---|---|---|---|---|---|---|
| | auxiliary | | | Current | Brightness | | Lifetime |
| | layer | host | Voltage | Density | (cd/m²) | Efficiency | T(95) |
| comparative | | comparative | 6.7 | 34.2 | 2500 | 7.3 | 63.5 |
| example(10) | | compound 5 | | | | | |
| comparative | | compound 3-6 | 6.2 | 24.5 | 2500 | 10.2 | 117.9 |
| example(11) | | | | | | | |
| comparative | | compound 3-7 | 6.0 | 21.6 | 2500 | 11.6 | 115.5 |
| example(12) | | | | | | | |
| comparative | | compound 3-8 | 6.1 | 18.0 | 2500 | 13.9 | 117.0 |
| example(13) | | | | | | | |
| comparative | | compound 3-101 | 5.9 | 14.1 | 2500 | 17.7 | 122.5 |
| example(14) | | | | | | | |
| example(70) | compound 2-1 | compound 3-6 | 6.1 | 16.3 | 2500 | 23.8 | 127.2 |
| example(71) | | compound 3-7 | 6.1 | 13.7 | 2500 | 25.7 | 125.2 |
| example(72) | | compound 3-8 | 6.0 | 12.3 | 2500 | 26.5 | 126.9 |
| example(73) | | compound 3-101 | 5.8 | 10.3 | 2500 | 27.2 | 129.6 |
| example(74) | compound 2-76 | compound 3-6 | 5.6 | 9.3 | 2500 | 28.5 | 137.8 |
| example(75) | | compound 3-7 | 5.7 | 8.6 | 2500 | 30.4 | 137.3 |
| example(76) | | compound 3-8 | 5.7 | 6.8 | 2500 | 32.2 | 138.7 |
| example(77) | | compound 3-101 | 5.4 | 6.0 | 2500 | 34.4 | 141.5 |
| example(78) | compound 2-88 | compound 3-6 | 5.7 | 10.5 | 2500 | 30.5 | 137.3 |
| example(79) | | compound 3-7 | 5.7 | 9.5 | 2500 | 31.7 | 135.6 |
| example(80) | | compound 3-8 | 5.7 | 7.3 | 2500 | 34.5 | 136.6 |
| example(81) | | compound 3-101 | 5.5 | 6.3 | 2500 | 36.1 | 139.3 |
| example(82) | compound 2-106 | compound 3-6 | 5.9 | 8.4 | 2500 | 27.3 | 136.4 |
| example(83) | | compound 3-7 | 5.8 | 7.8 | 2500 | 29.4 | 135.8 |
| example(84) | | compound 3-8 | 5.9 | 6.2 | 2500 | 31.1 | 138.2 |
| example(85) | | compound 3-101 | 5.7 | 5.6 | 2500 | 33.7 | 140.4 |

As can be seen from the results of Table 10, when the compound of the present invention represented by Formula (1) is used as a material for the emitting auxiliary layer and the compound of the present invention represented by Formula (2) is used as a phosphorescent host material (Examples 70 to 85), the driving voltage, the efficiency, and the lifetime were significantly improved as compared with the device using a single material (Comparative Examples 10 to 14).

In detail, in Comparative Examples 10 to 14 using the compound of the present invention represented by the formula (2) or Comparative Compound 5 alone as a phosphorescent host, Comparative Examples 11 to 14 using the compounds of the present invention (3-6, 3-7, 3-8, and 3-101) exhibited higher efficiency and longer lifetime than Comparative Example 1 using Comparative Compound 5.

It was also confirmed that Examples 70 to 85 using the compounds of Formula (1) and Formula (2) of the present invention, as the emitting auxiliary layer and the phosphorescent host, respectively, show significantly higher efficiency, lifetime and low driving voltage than the Comparative Examples 10 to 14.

In general, there is an injection barrier between HTL and EML, so that the hole can not be transferred easily, and the charge balance is not matched so that the driving voltage is increased. Therefore, it is considered that the charge balance in the emitting layer of the hole and electron is adjusted by the introduction of the emitting auxiliary layer having a proper HOMO level between HTL and EML when the emitting auxiliary layer of the present invention is applied.

Specially, a compound in which at least one of Ar¹, Ar², Ar³, Ar⁴, L¹, L², L³, L⁴, and L⁵ is substituted with a dibenzothiophene or dibenzofuran exhibited remarkably higher refractive index and higher Tg as compared with the case where the substituent of the general aryl group was substituted, so that the efficiency and the thermal stability were improved, and it was judged that the compound showed improved device results.

The compound of the present invention represented by Formula (1) suitably align the barriers of HTL and EML so that the device has a feature of high hole mobility, and compounds of the present invention represented by Formula (2) have characteristics of not only fast electron mobility but also hole stability and high T1 as compared with Comparative compound 6. Therefore, the combination of the two makes it possible to move more holes to the emissive layer quickly and easily. As a result, the charge balance in the emitting layer of holes and electrons is increased, so that light emission is well performed inside the emitting layer rather than at the interface of the hole transport layer, and therefore the deterioration in the ITO and HTL interface is also reduced, thereby maximizing the driving voltage, efficiency and life span of the device. That is, it is judged that the combination of the compound of the present invention represented by Formula (1) and the compound of the present invention represented by Formula (2) is electrochemically synergistic to improve the performance of the device as a whole.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas included within the scope equivalent to the claims belong to the present invention.

What is claimed is:

1. An organic electric element comprising a first electrode, a second electrode, and an organic material layer formed between the first electrode and the second electrode,
wherein the organic material layer comprises an emitting layer, and the emitting layer is a phosphorescent light emitting layer comprising a first host compound represented by Formula (1) and a second host compound represented by Formula (2): 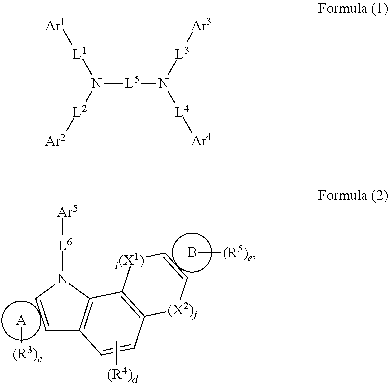
with the proviso that a compound of Formula (3-20) is excluded from Formula (1); 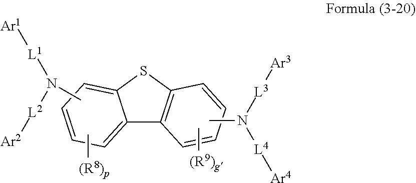
wherein:
1) Ar¹, Ar², Ar³, Ar⁴ and Ar⁵ are each independently selected from the group consisting of a C₆-C₆₀ aryl group; a C₂-C₆₀ heterocyclic group including at least one hetero atom of O, N, S, Si or P; a fused ring group of a C₃-C₆₀ aliphatic ring and a C₆-C₆₀ aromatic ring; a C₁-C₅₀) alkyl group; a C₂-C₂₀ alkenyl group; a C₂-C₂₀ alkynyl group; a C₁-C₃₀ alkoxyl group; and a C₆-C₃₀ aryloxy group; and Ar¹ and Ar² or Ar³ and Ar⁴ may be bonded to each other to form a ring, with the proviso that where Ar¹ and Ar² form a ring, the compound of Formula (1) is represented by Formula (3): 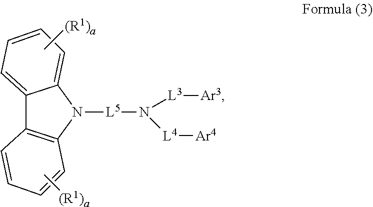
2) a and b are each independently an integer of 0 to 4, c and e are an integer of 0 to 10, and d is an integer of 0 to 2, and f and g′ are integer of 0 to 3,
3) R¹, R², R³, R⁴ and R⁵ are the same or different from each other, and are each independently selected from the group consisting of hydrogen; deuterium; halogen; a C₆-C₆₀ aryl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; and a C₆-C₃₀ aryloxy group; or in case a, b, c, d, e, f and g′ are 2 or more, and R¹, R², R³, R⁴ and R⁵, R⁸ and R⁹ are each in plural being the same or different, and a plurality of R¹ or a plurality of R² or a plurality of R³ or a plurality of R⁴ or a plurality of R⁵ or a plurality of R⁸ or a plurality of R⁹, or two adjacent R⁸ and R⁹ may be bonded to each other to form an aromatic or heteroaromatic ring,
4) L¹, L², L³, L⁴, L⁵ and L⁶ are each independently selected from the group consisting of a single bond; a C₆-C₆₀ arylene group; a fused ring group of a C₃-C₆₀ aliphatic ring and a C₆-C₆₀ aromatic ring; and a C₂-C₆₀ heterocyclic group; provided that L⁵ is not a single bond,
5) A and B are each independently a C₆-C₆₀ aryl group or a C₂-C₂₀ heterocyclic group, provided that when both A and B are a substituted or unsubstituted C₆ aryl group (phenyl group), d is 2, and R⁴s are bonded to each other to form an aromatic or heterocyclic group,
6) i and j are 0 or 1, with the proviso that i+j is 1 or more, and when i or j is 0, it means a direct bond,
7) X¹ and X² are each independently N-L⁷-Ar⁶, O, S, or CR⁶R⁷, wherein L⁷ is the same as L¹ to L⁴ or L⁶, wherein Ar⁶ is the same as Ar¹ to Ar⁵, and R⁶ and R⁷ are each independently hydrogen; a C₆-C₆₀ aryl group; a C₂-C₆₀ heterocyclic group; or a C₁-C₅₀ alkyl group and R⁶ and R⁷ may combine to each other to form a spiro ring,
wherein, the aryl group, arylene group, heterocyclic group, fused ring group, alkyl group, alkenyl group, alkoxy group and aryloxy group may be substituted with one or more substituents selected from the group consisting of deuterium; halogen; a silane group substituted or unsubstituted with C₁-C₂₀ alkyl group or C₆-C₂₀ aryl group; siloxane group; boron group; germanium group; cyano group; nitro group; a C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; C₂-C₂₀ heterocyclic group; C₃-C₂₀ cycloalkyl group; C₇-C₂₀ arylalkyl group; and C₈-C₂₀ arylalkenyl group; wherein the substituents may combine each other and form a saturated or unsaturated ring, wherein the term ‘ring’ means C₃-C₆₀ aliphatic ring or C₆-C₆₀ aromatic ring or a C₂-C₆₀ heterocyclic group or a fused ring formed by the combination of thereof and includes a saturated or unsaturated ring.

2. The organic electric element according to claim 1, wherein L¹, L², L³, L⁴ and L⁵ in Formula (1) are each independently any one of the following Formulas (A-1) to (A-13): 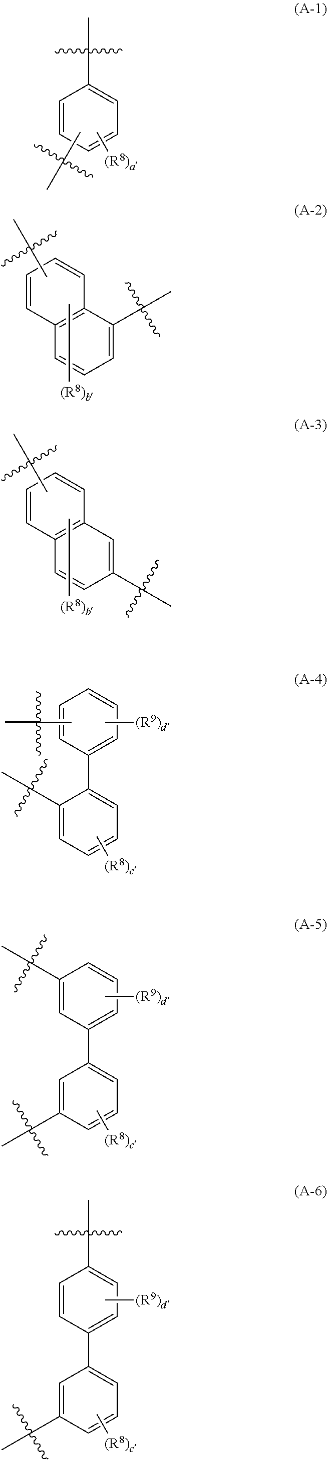 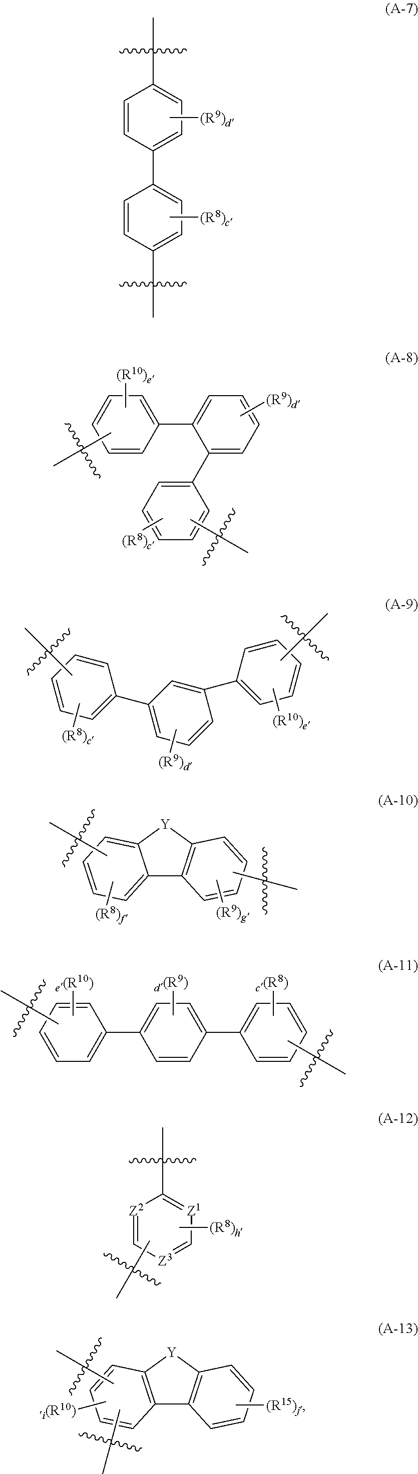
wherein:
1) a′, c′, d′ and e′ are an integer of 0 to 4; and b′ is an integer of 0 to 6; and f and g′ are an integer of 0 to 3; and h′ is an integer of 0 or 1; and i′ is an integer of 0 to 2; and j′ is an integer of 0 to 4;
2) R⁸, R⁹, R¹⁰ and R¹⁵ are the same or different from each other, and are each independently selected from the group consisting of hydrogen; deuterium; halogen; a C₆-C₂₀ aryl group; a C₂-C₂₀ heterocyclic group including at least one heteroatom of O, N, S, Si or P; a fused ring group of a C₃-C₂₀ aliphatic ring and a C₆-C₂₀ aromatic ring; a C₁-C₂₀ alkyl group; a C₂-C₂₀ alkenyl group; a C₂-C₂₀ alkynyl group; a C₁-C₂₀ alkoxyl group; a C₆-C₃₀ aryloxy group; and wherein e′, f′, g′, i′ and j′ are 2 or more, R⁸, R⁹, R¹⁰ and R¹⁵ are the same or different from each other, and a plurality of R⁸ or a plurality of R⁹ or a plurality of R¹⁰ or a plurality of R¹⁵ or two adjacent R⁸ and R⁹, or R⁹ and R¹⁰, or R¹⁰ and R¹⁵ may be combined to each other to form an aromatic ring or heteroaromatic ring,
3) Y is N-L⁸-Ar⁷, O, S or CR¹¹R¹², wherein L⁸ is the same as L¹ to L⁶ defined in claim 1, Ar¹ is the same as Ar¹ to AO defined in claim 1, and R^{H} and R¹² are the same as R⁶ and R⁷ defined in claim 1,
4) Z¹, Z² and Z³ are CR¹³ or N and at least one is N, and R¹³ is the same as R⁸ and R¹⁰ defined in claim 1.

3. The organic electric element according to claim 1, wherein the first host compound represented by Formula (1) is represented by any one of the following Formulas (3-1) to (3-19): 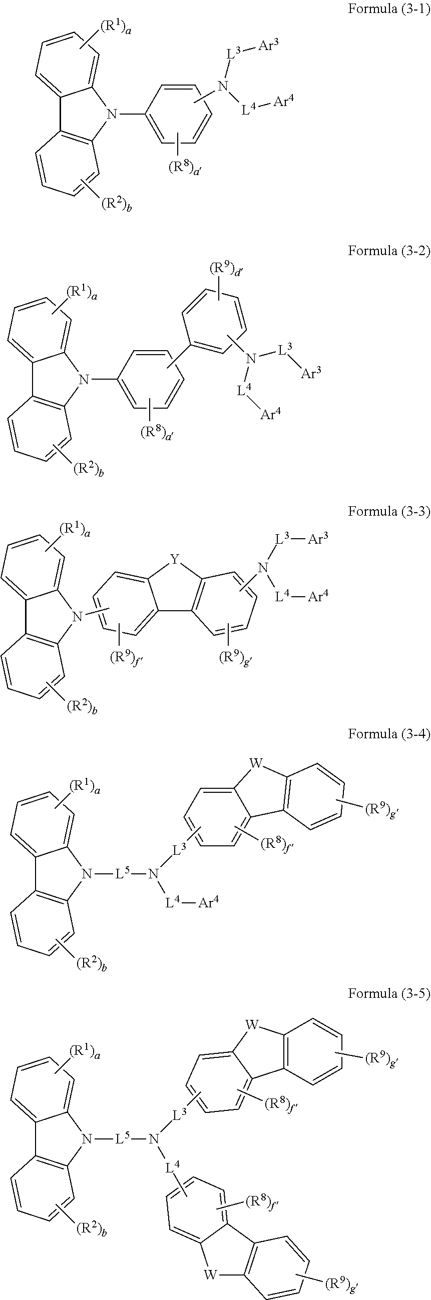 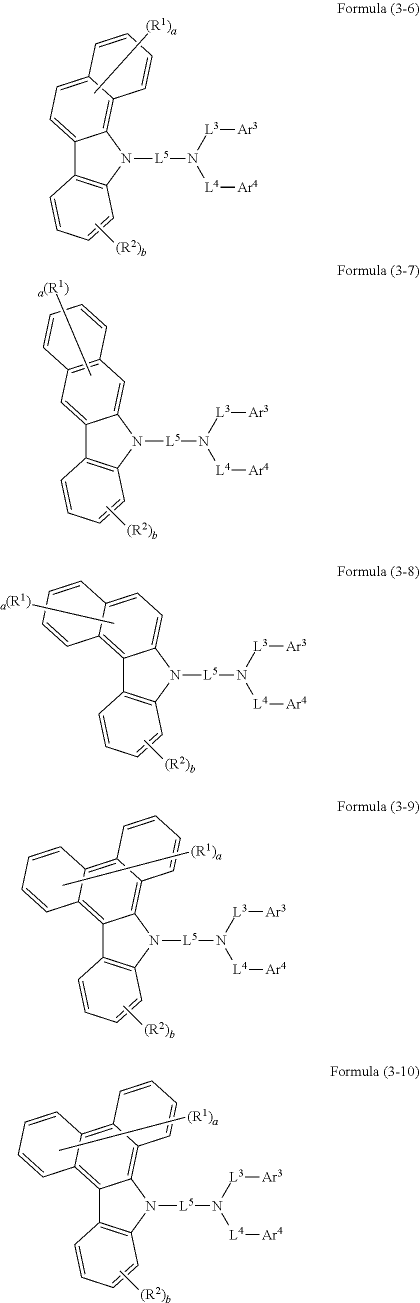 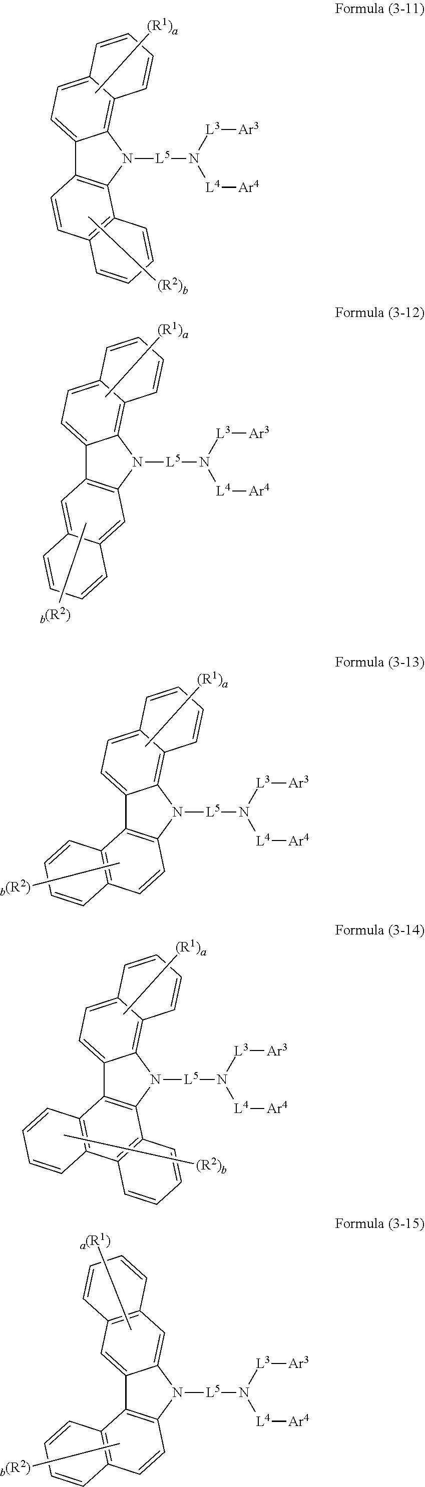 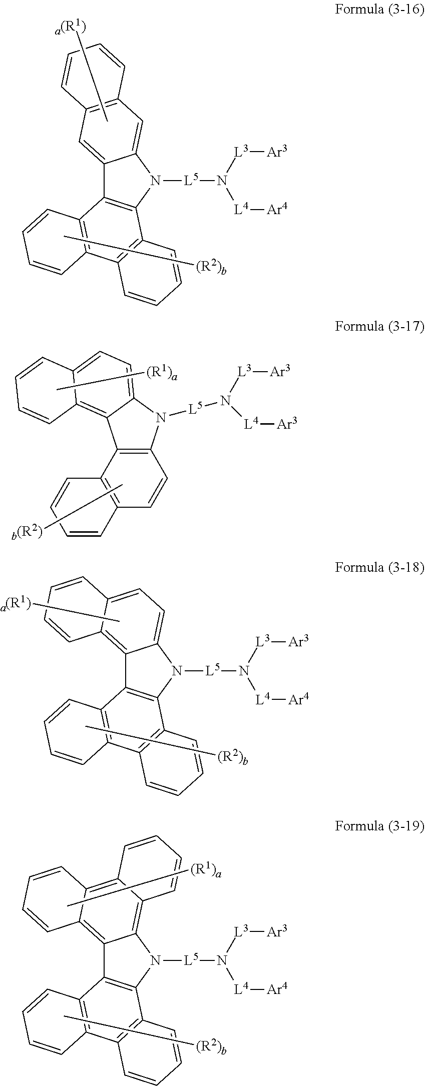
wherein:
1) L³, L⁴, L⁵, Ar³ and Ar⁴ are the same as defined in claim 1,
2) a and b are each independently integer of 0 to 4,
3) R¹, R², R⁸ and R⁹ are the same or different from each other, and are each independently selected from the group consisting of hydrogen; deuterium; halogen; a C₆-C₆₀ aryl group; a C₂-C₂₀ heterocyclic group including at least one heteroatom of O, N, S, Si or P; a fused ring group of a C₃-C₂₀ aliphatic ring and a C₆-C₂₀ aromatic ring; a C₁-C₂₀ alkyl group; a C₂-C₂₀ alkenyl group; a C₂-C₂₀ alkynyl group; a C₁-C₂₀ alkoxyl group; and a C₆-C₃₀ aryloxy group; or in case a, b, a′, d′, f′ or g′ are 2 or more, R¹, R², R⁸ and R⁹ are each in plural being the same or different, and a plurality of R¹ or a plurality of R² or a plurality of R⁸ or a plurality of R⁹, or adjacent R¹ and R² or R⁸ and R⁹ may be bonded to each other to form an aromatic or heteroaromatic ring,
4) a′ and d′ are an integer of 0 to 4; and f and g′ are an integer of 0 to 3;
5) Y is N-L⁸-Ar⁷, O, S or CR¹¹R¹²,
6) W is N-L⁸-Ar⁷, O, S or CR¹¹R¹², wherein L⁸ is the same as L¹ to L⁶ defined in claim 1, Ar⁷ is the same as Ar¹ to Ar⁵ defined in claim 1, and R^{H} and R¹² are the same as R⁶ and R⁷ defined in claim 1.

4. The organic electric element according to claim 1, wherein both Ar³ and Ar⁴ in Formula (1) are a C₆-C₂₄ aryl group.

5. The organic electric element according to claim 1, wherein at least one of Ar³ and Ar⁴ in Formula (1) is a dibenzothiophene or dibenzofuran compound.

6. The organic electric element according to claim 1, wherein at least one of L¹, L², L³, L⁴ and L⁵ in Formula (1) is substituted on an m(meta)-position.

7. The organic electric element according to claim 1, wherein the second host compound represented by Formula (2) is represented by the following Formula (4) or (5): 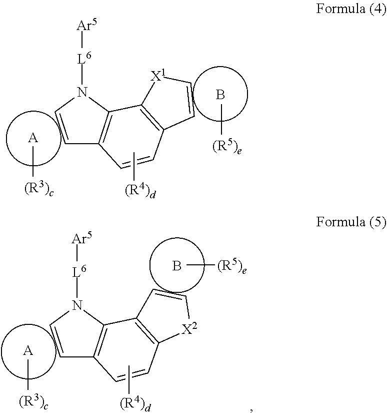
wherein R³, R⁴, R⁵, L⁶, Ar⁵, X¹, X², A, B, c, d, and e are the same as defined in claim 1.

8. The organic electric element according to claim 1, wherein A and B in Formula (2) are selected from the group consisting of the following Formulas (B-1) to (B-7): 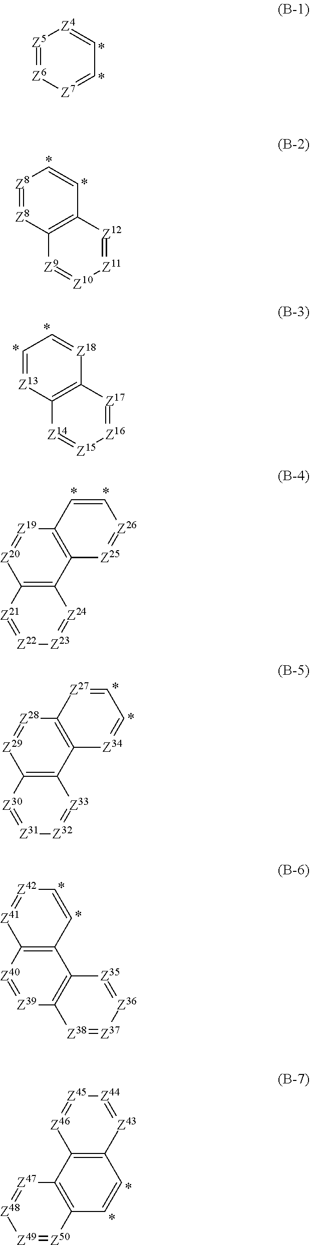
wherein:
1) Z⁴ to Z⁵⁰ are CR¹⁴ or N,
2) R¹⁴ is the same as R³ to R⁵ defined in claim 1,
3) * indicates the position to be condensed.

9. The organic electric element according to claim 1, wherein the second host compound represented by Formula (2) comprises a compound represented by any of the following Formulas (4-1) to (4-36): 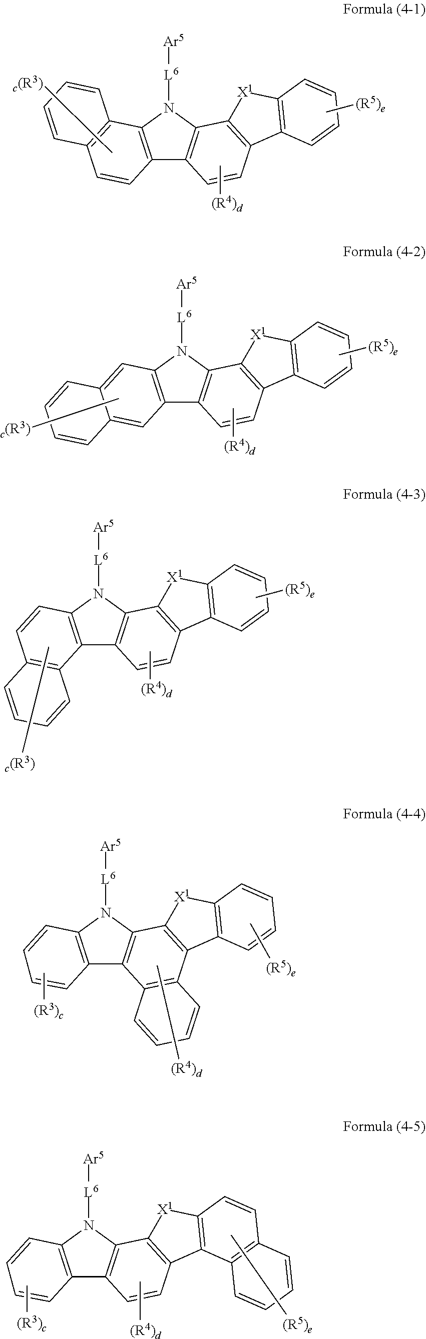 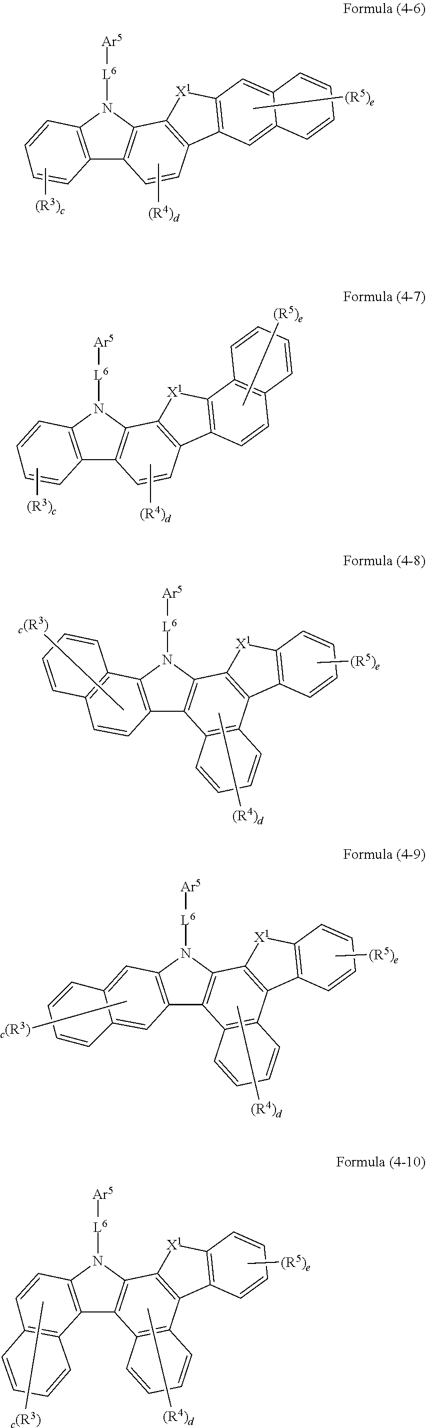 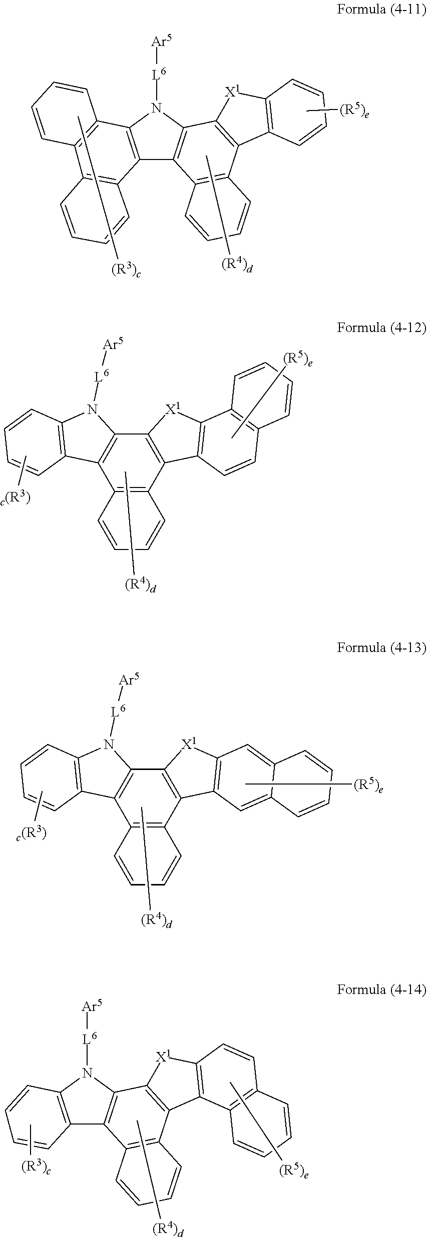 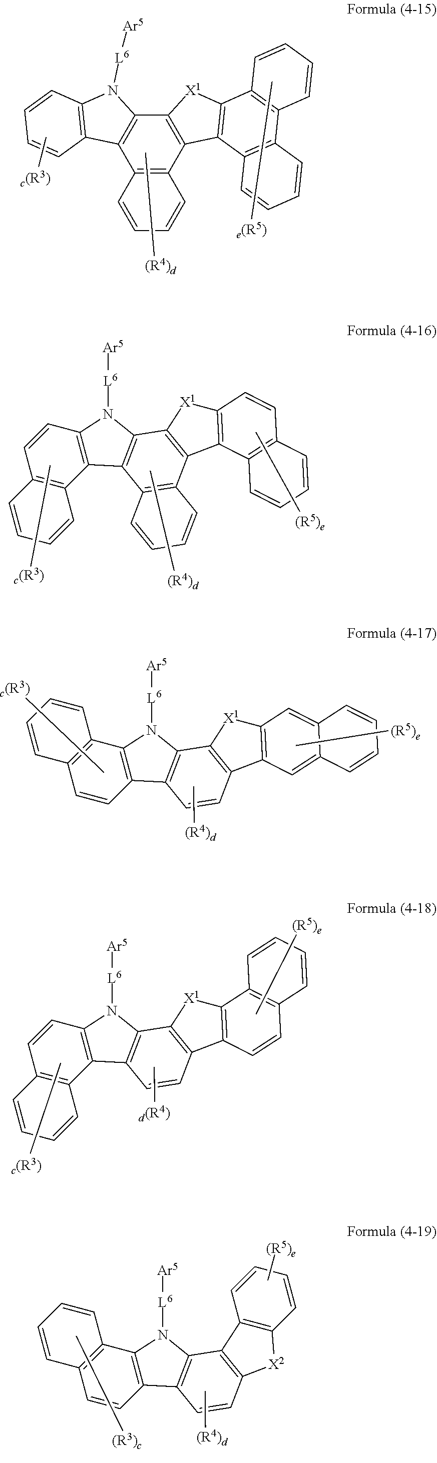 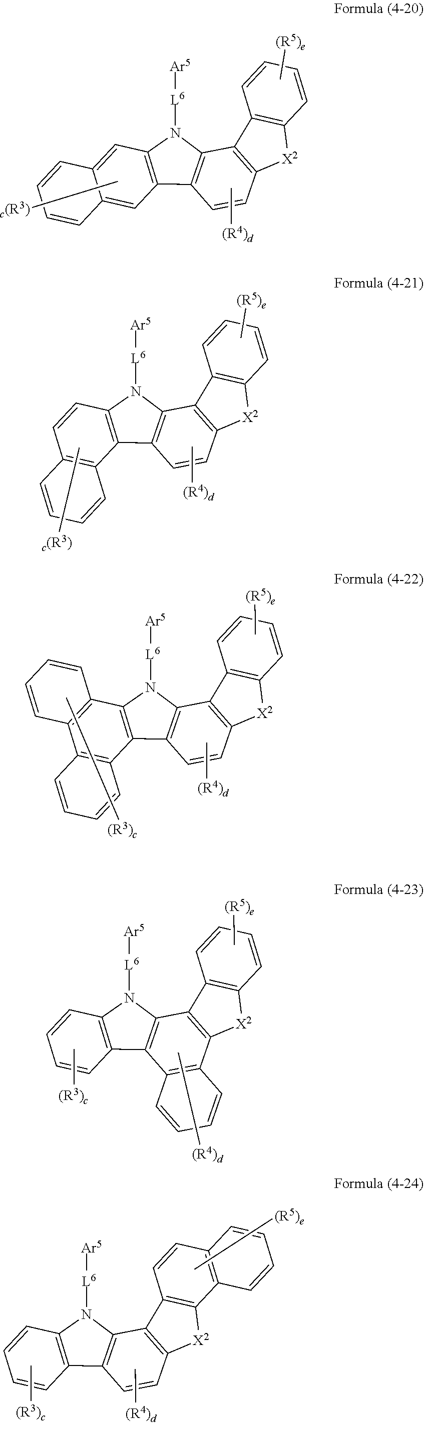 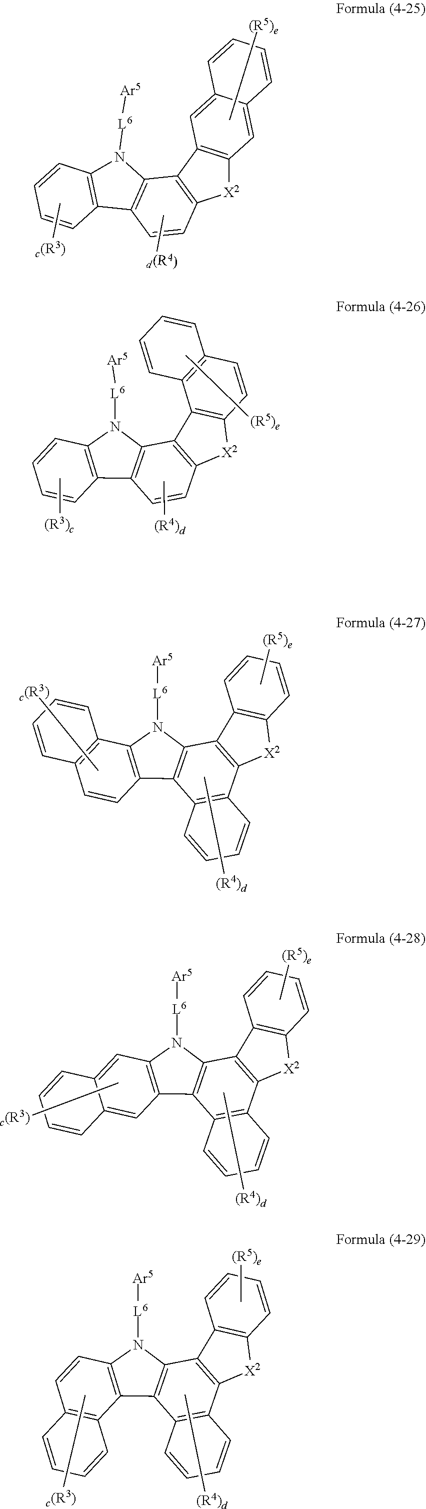 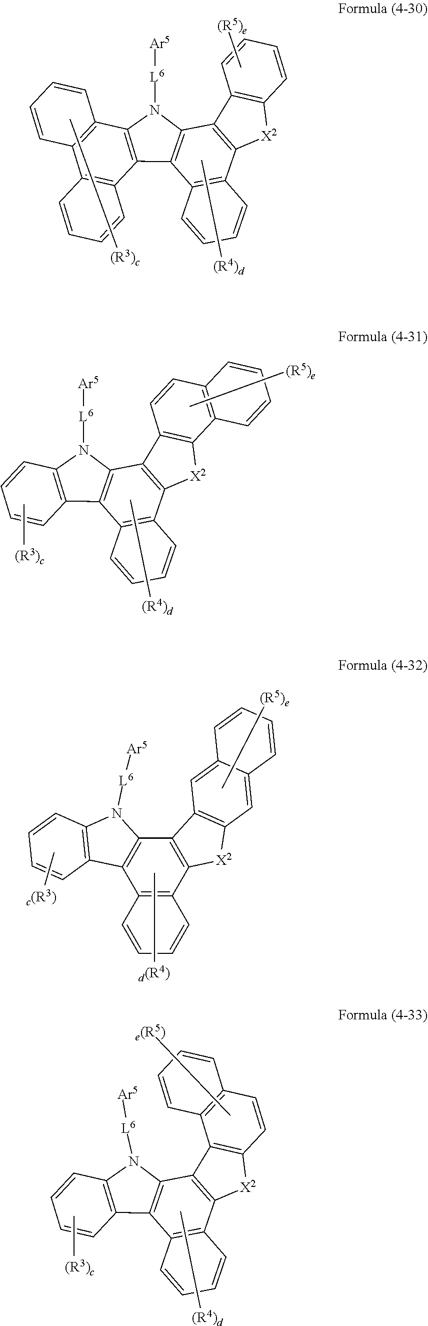 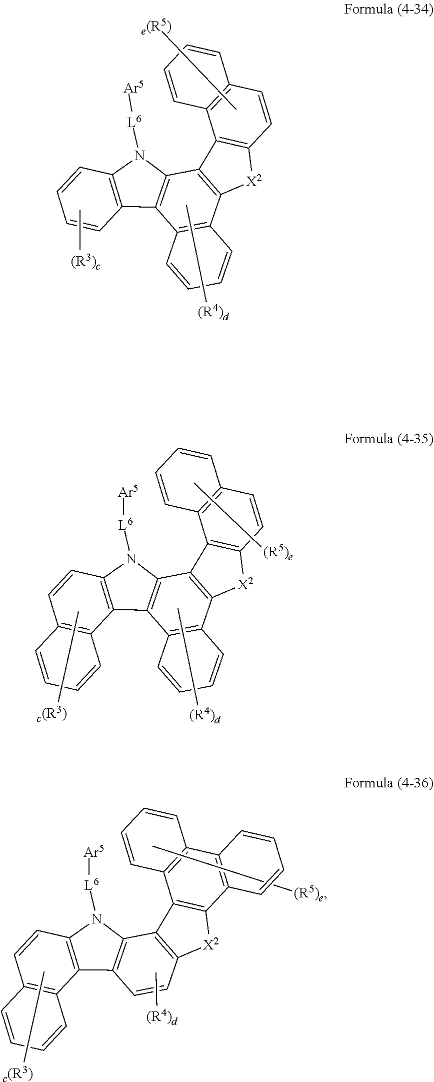
wherein Ar⁵, L⁶, R³, R⁴, R⁵, X₁, X², c and e are the same as defined in claim 1, and d is any one of integer of 0 to 4.

10. The organic electric element according to claim 1, wherein the second host compound represented by Formula (2) comprises compounds represented by the following Formulas (6-1) to (6-8): 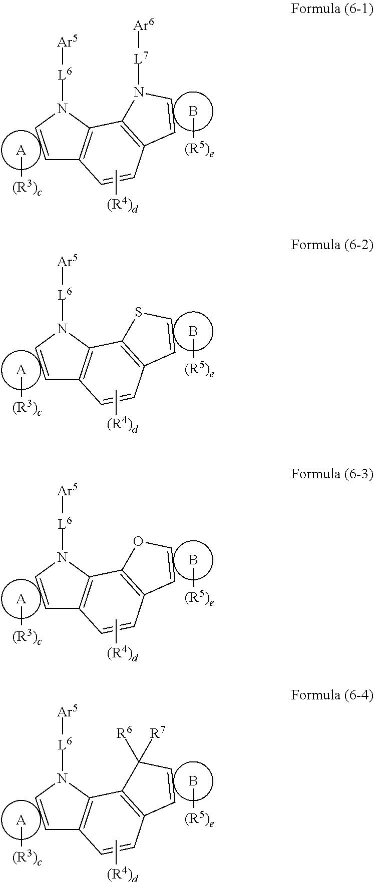 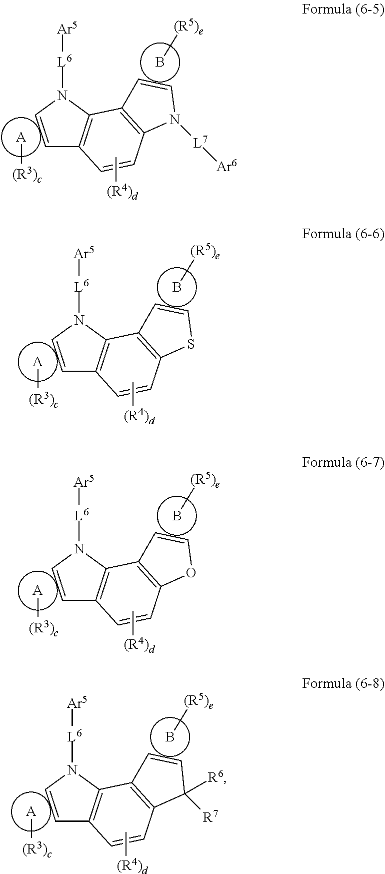
wherein R³, R⁴, R⁵, R⁶, R⁷, L⁶, L⁷, Ar⁵, Ar⁶, c, d, e, A and B are the same as defined in claim 1.

11. The organic electric element according to claim 1, wherein the first host compound represented by Formula (1) comprises the following Compounds 1-1 to 1-60 and 2-1 to 2-49, 2-52 to 2-54, 2-56, 2-59 to 2-70, 2-101 to 2-105:

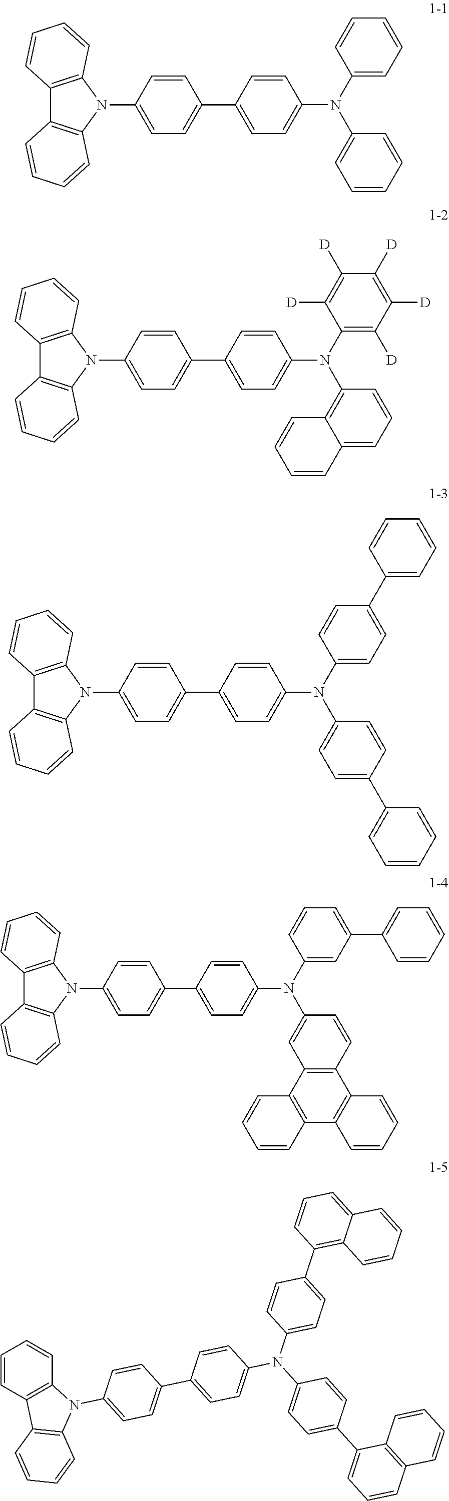

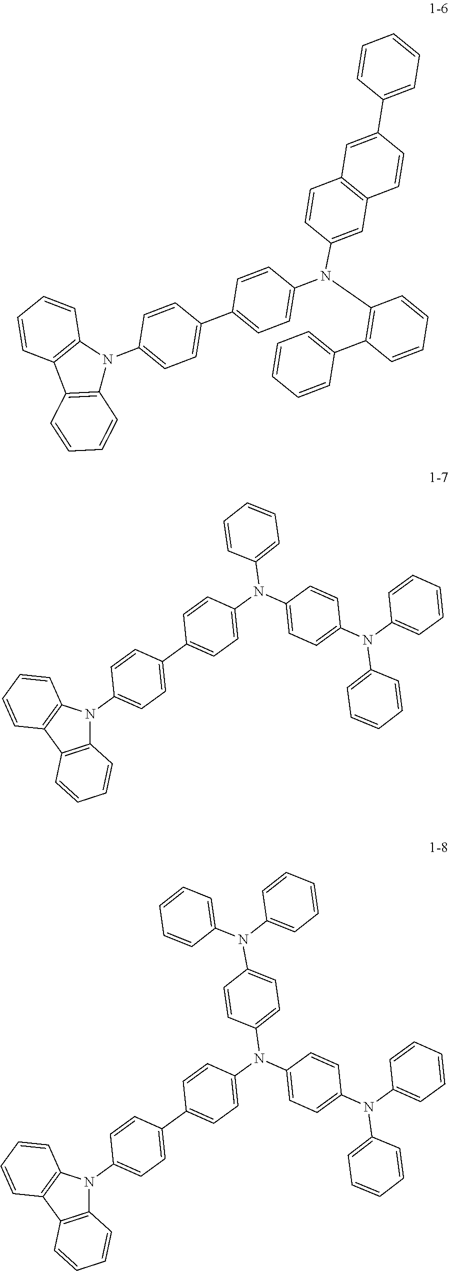 [SMILES: c1ccc(-c2ccc3cc(N(c4ccc(-c5ccc(-n6c7ccccc7c7ccccc76)cc5)cc4)c4ccccc4-c4ccccc4)ccc3c2)cc1 ; c1ccc(N(c2ccccc2)c2ccc(N(c3ccccc3)c3ccc(-c4ccc(-n5c6ccccc6c6ccccc65)cc4)cc3)cc2)cc1 ; c1ccc(N(c2ccccc2)c2ccc(N(c3ccc(-c4ccc(-n5c6ccccc6c6ccccc65)cc4)cc3)c3ccc(N(c4ccccc4)c4ccccc4)cc3)cc2)cc1]

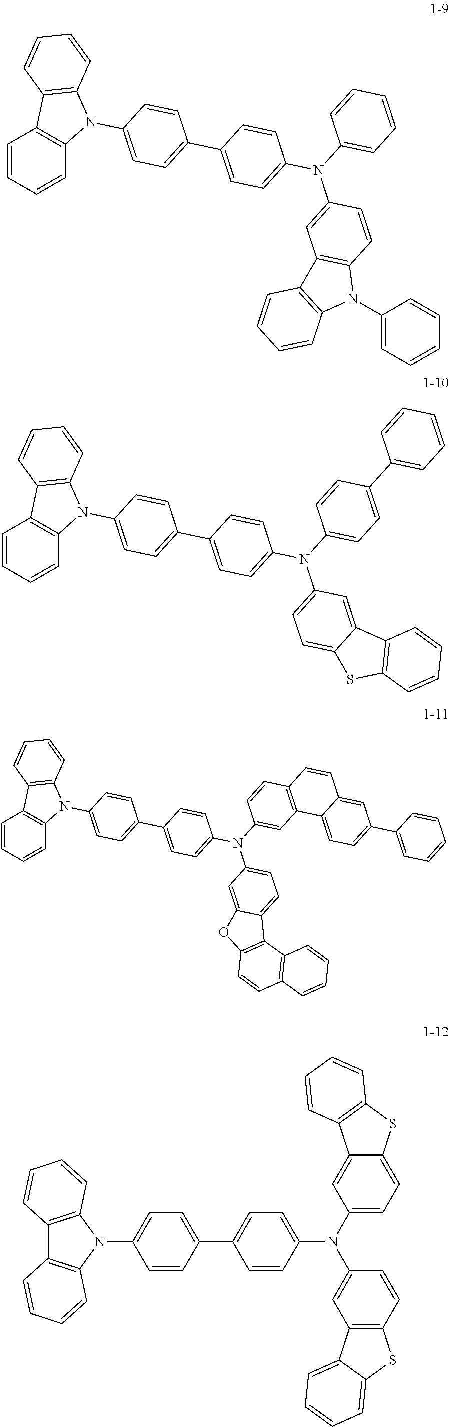

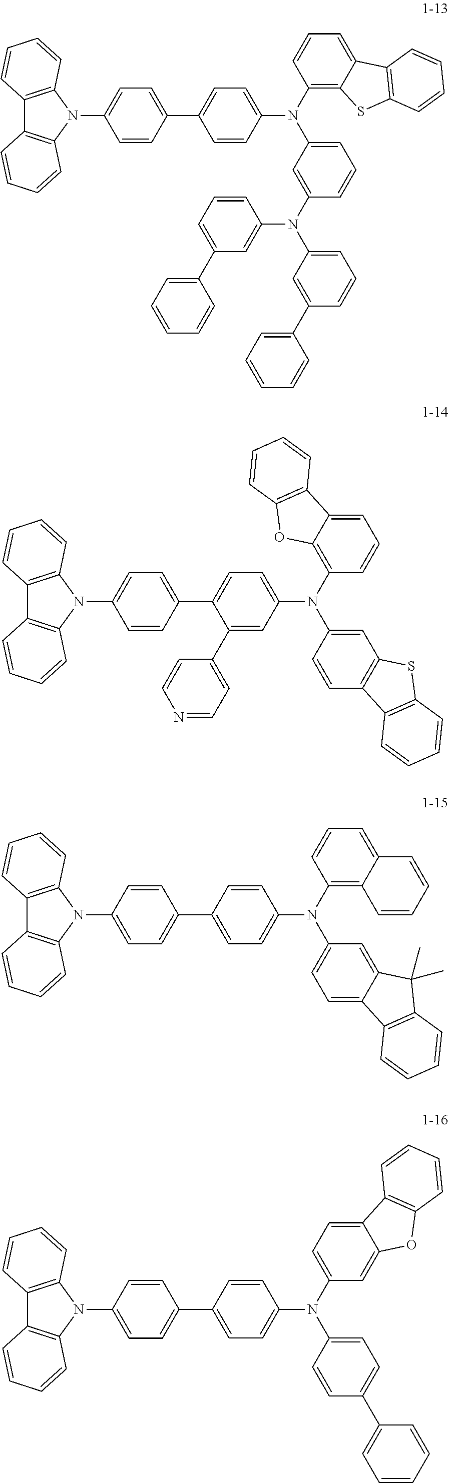 [SMILES: c1ccc(-c2cccc(N(c3cccc(-c4ccccc4)c3)c3cccc(N(c4ccc(-c5ccc(-n6c7ccccc7c7ccccc76)cc5)cc4)c4cccc5c4sc4ccccc45)c3)c2)cc1 ; c1ccc2c(c1)oc1c(N(c3ccc(-c4ccc(-n5c6ccccc6c6ccccc65)cc4)c(-c4ccncc4)c3)c3ccc4c(c3)sc3ccccc34)cccc12 ; CC1(C)c2ccccc2-c2ccc(N(c3ccc(-c4ccc(-n5c6ccccc6c6ccccc65)cc4)cc3)c3cccc4ccccc34)cc21 ; c1ccc(-c2ccc(N(c3ccc(-c4ccc(-n5c6ccccc6c6ccccc65)cc4)cc3)c3ccc4c(c3)oc3ccccc34)cc2)cc1]

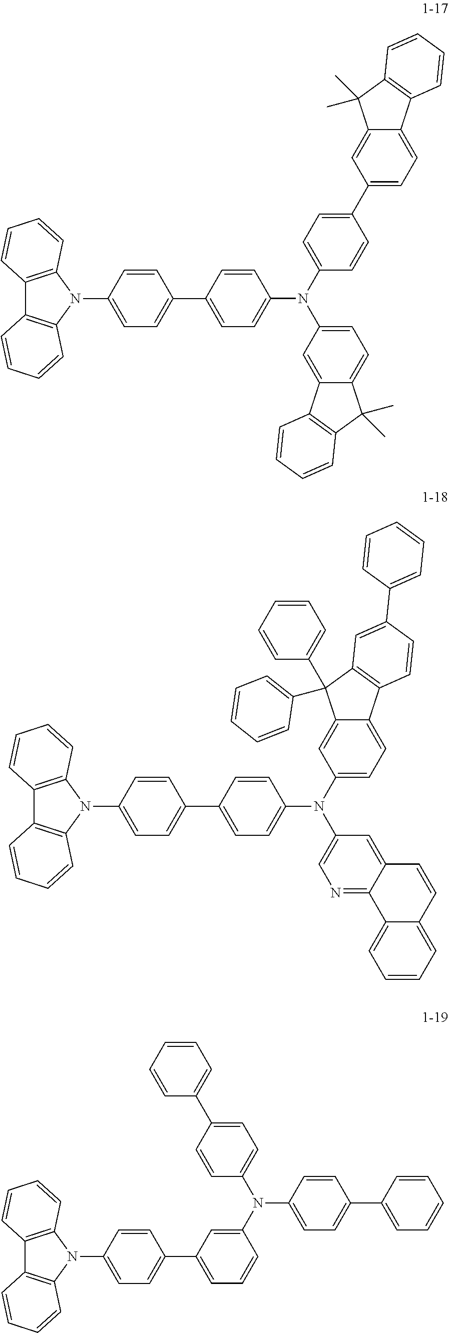 [SMILES: CC1(C)c2ccccc2-c2cc(N(c3ccc(-c4ccc(-n5c6ccccc6c6ccccc65)cc4)cc3)c3ccc(-c4ccc5c(c4)C(C)(C)c4ccccc4-5)cc3)ccc21 ; c1ccc(-c2ccc3c(c2)C(c2ccccc2)(c2ccccc2)c2cc(N(c4ccc(-c5ccc(-n6c7ccccc7c7ccccc76)cc5)cc4)c4cnc5c(ccc6ccccc65)c4)ccc2-3)cc1 ; c1ccc(-c2ccc(N(c3ccc(-c4ccccc4)cc3)c3cccc(-c4ccc(-n5c6ccccc6c6ccccc65)cc4)c3)cc2)cc1]

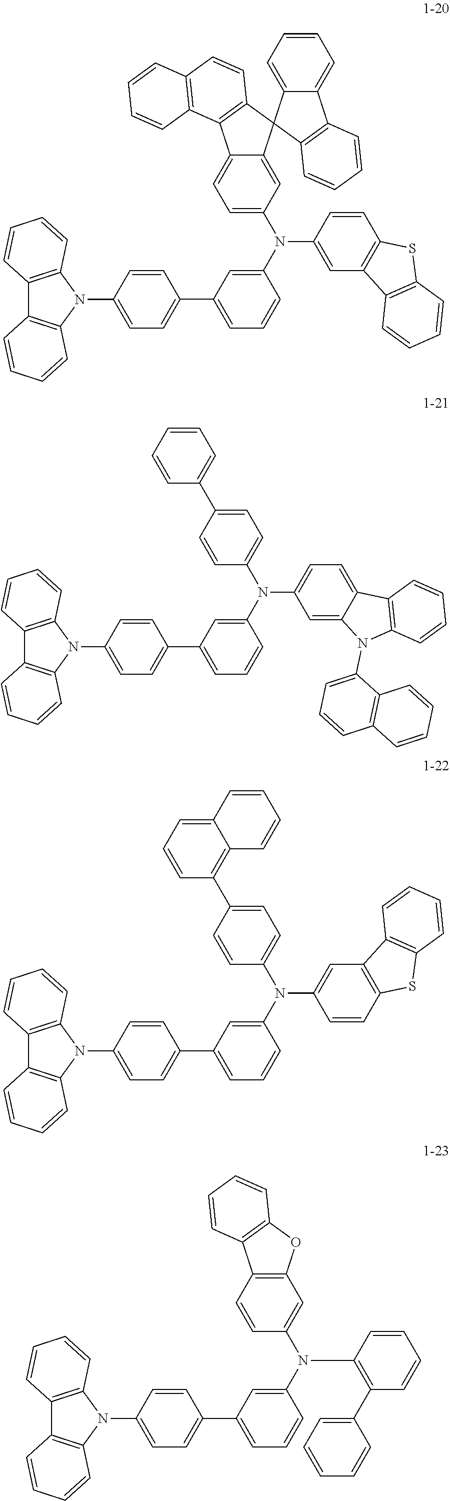 [SMILES: c1cc(-c2ccc(-n3c4ccccc4c4ccccc43)cc2)cc(N(c2ccc3c(c2)C2(c4ccccc4-c4ccccc42)c2ccc4ccccc4c2-3)c2ccc3sc4ccccc4c3c2)c1 ; c1ccc(-c2ccc(N(c3cccc(-c4ccc(-n5c6ccccc6c6ccccc65)cc4)c3)c3ccc4c5ccccc5n(-c5cccc6ccccc56)c4c3)cc2)cc1 ; c1cc(-c2ccc(-n3c4ccccc4c4ccccc43)cc2)cc(N(c2ccc(-c3cccc4ccccc34)cc2)c2ccc3sc4ccccc4c3c2)c1 ; c1ccc(-c2ccccc2N(c2cccc(-c3ccc(-n4c5ccccc5c5ccccc54)cc3)c2)c2ccc3c(c2)oc2ccccc23)cc1]

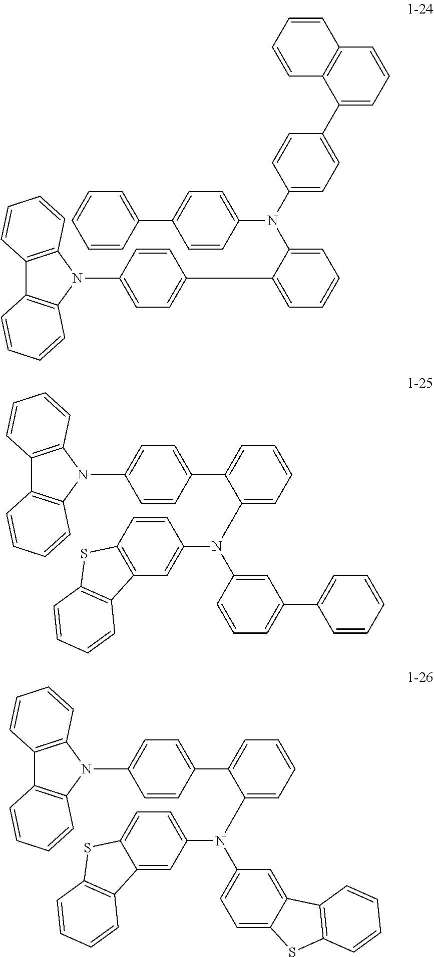

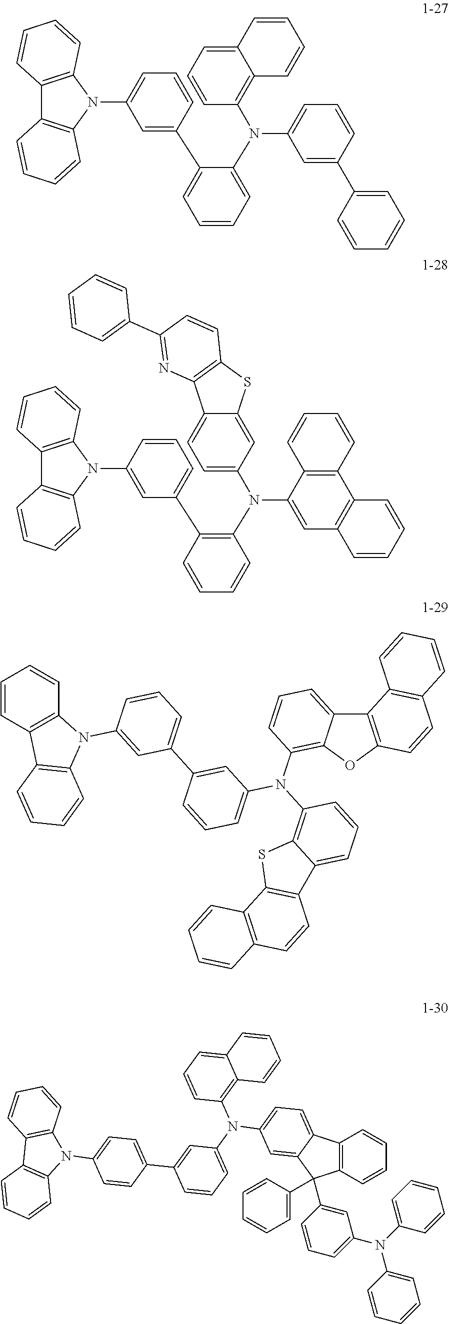 [SMILES: c1ccc(-c2cccc(N(c3ccccc3-c3cccc(-n4c5ccccc5c5ccccc54)c3)c3cccc4ccccc34)c2)cc1 ; c1ccc(-c2ccc3sc4cc(N(c5ccccc5-c5cccc(-n6c7ccccc7c7ccccc76)c5)c5cc6ccccc6c6ccccc56)ccc4c3n2)cc1 ; c1cc(-c2cccc(-n3c4ccccc4c4ccccc43)c2)cc(N(c2cccc3c2oc2ccc4ccccc4c23)c2cccc3c2sc2c4ccccc4ccc32)c1 ; c1ccc(N(c2ccccc2)c2cccc(C3(c4ccccc4)c4ccccc4-c4ccc(N(c5cccc(-c6ccc(-n7c8ccccc8c8ccccc87)cc6)c5)c5cccc6ccccc56)cc43)c2)cc1]

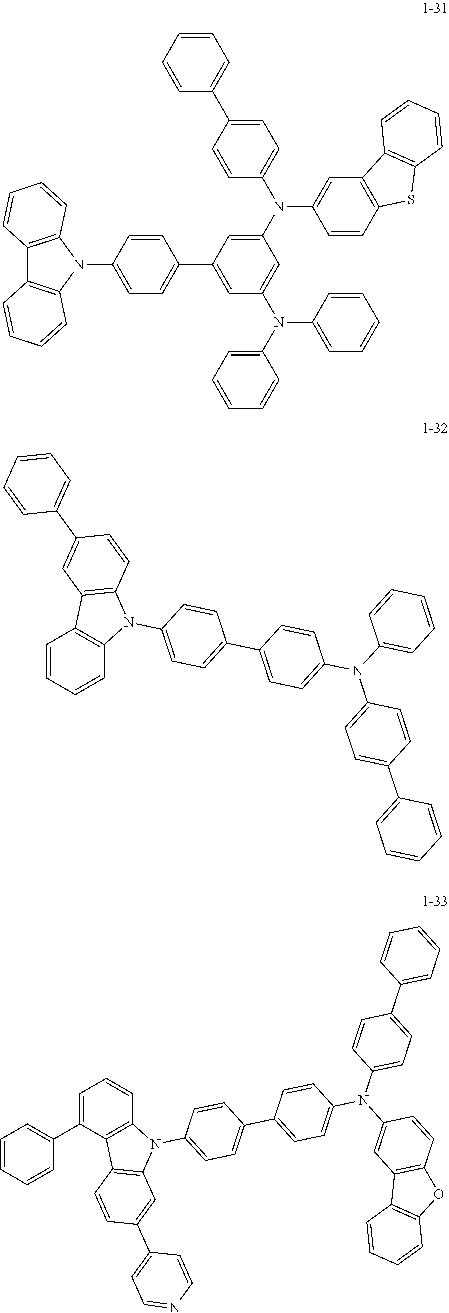 [SMILES: c1ccc(-c2ccc(N(c3cc(-c4ccc(-n5c6ccccc6c6ccccc65)cc4)cc(N(c4ccccc4)c4ccccc4)c3)c3ccc4sc5ccccc5c4c3)cc2)cc1 ; c1ccc(-c2ccc(N(c3ccccc3)c3ccc(-c4ccc(-n5c6ccccc6c6cc(-c7ccccc7)ccc65)cc4)cc3)cc2)cc1 ; c1ccc(-c2ccc(N(c3ccc(-c4ccc(-n5c6cc(-c7ccncc7)ccc6c6c(-c7ccccc7)cccc65)cc4)cc3)c3ccc4oc5ccccc5c4c3)cc2)cc1]

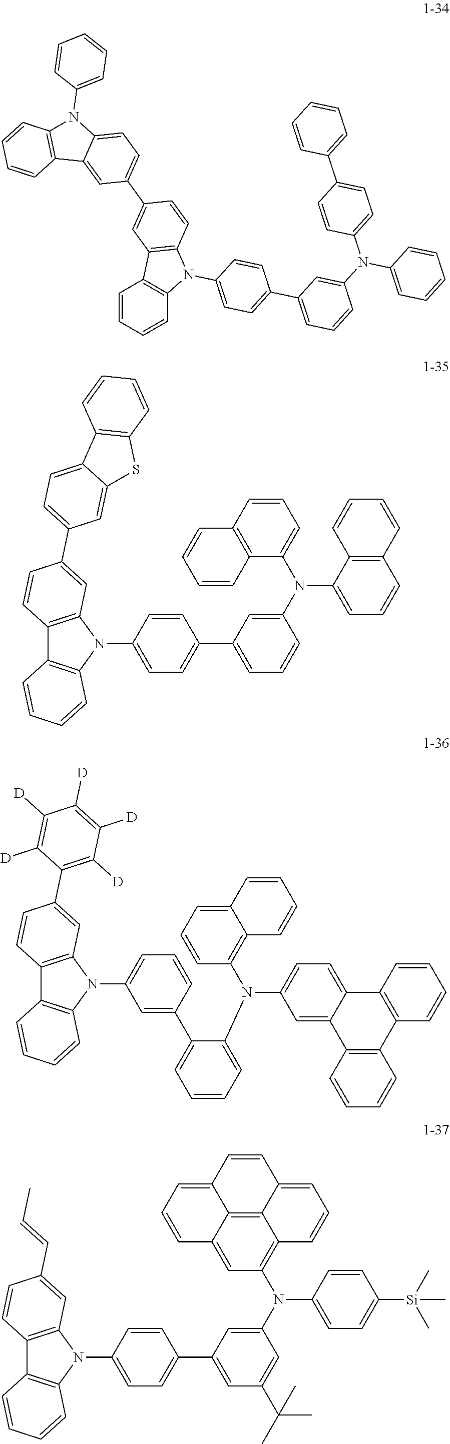

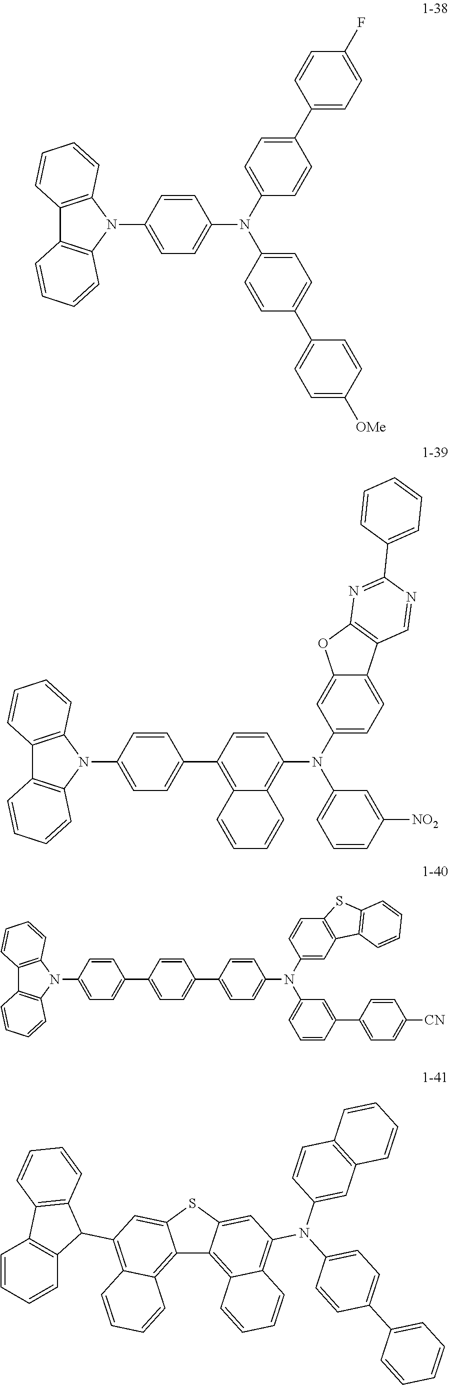 [SMILES: COc1ccc(-c2ccc(N(c3ccc(-c4ccc(F)cc4)cc3)c3ccc(-n4c5ccccc5c5ccccc54)cc3)cc2)cc1 ; O=[N+]([O-])c1cccc(N(c2ccc3c(c2)oc2nc(-c4ccccc4)ncc23)c2ccc(-c3ccc(-n4c5ccccc5c5ccccc54)cc3)c3ccccc23)c1 ; N#Cc1ccc(-c2cccc(N(c3ccc(-c4ccc(-c5ccc(-n6c7ccccc7c7ccccc76)cc5)cc4)cc3)c3ccc4sc5ccccc5c4c3)c2)cc1 ; c1ccc(-c2ccc(N(c3ccc4ccccc4c3)c3cc4sc5cc(C6c7ccccc7-c7ccccc76)c6ccccc6c5c4c4ccccc34)cc2)cc1]

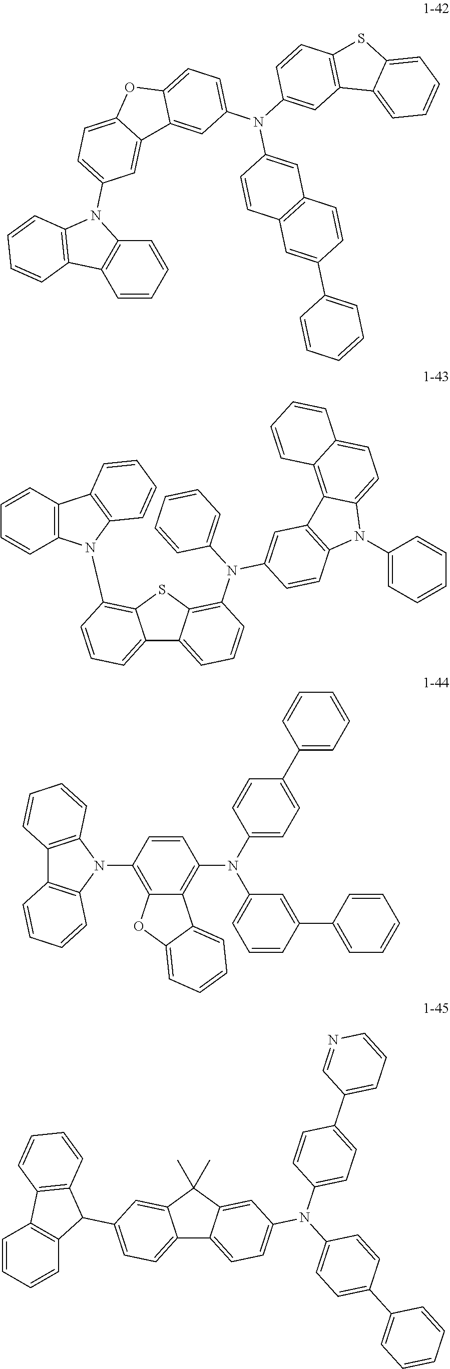 [SMILES: c1ccc(-c2ccc3cc(N(c4ccc5oc6ccc(-n7c8ccccc8c8ccccc87)cc6c5c4)c4ccc5sc6ccccc6c5c4)ccc3c2)cc1 ; c1ccc(N(c2ccc3c(c2)c2c4ccccc4ccc2n3-c2ccccc2)c2cccc3c2sc2c(-n4c5ccccc5c5ccccc54)cccc23)cc1 ; c1ccc(-c2ccc(N(c3cccc(-c4ccccc4)c3)c3ccc(-n4c5ccccc5c5ccccc54)c4oc5ccccc5c34)cc2)cc1 ; CC1(C)c2cc(C3c4ccccc4-c4ccccc43)ccc2-c2ccc(N(c3ccc(-c4ccccc4)cc3)c3ccc(-c4cccnc4)cc3)cc21]

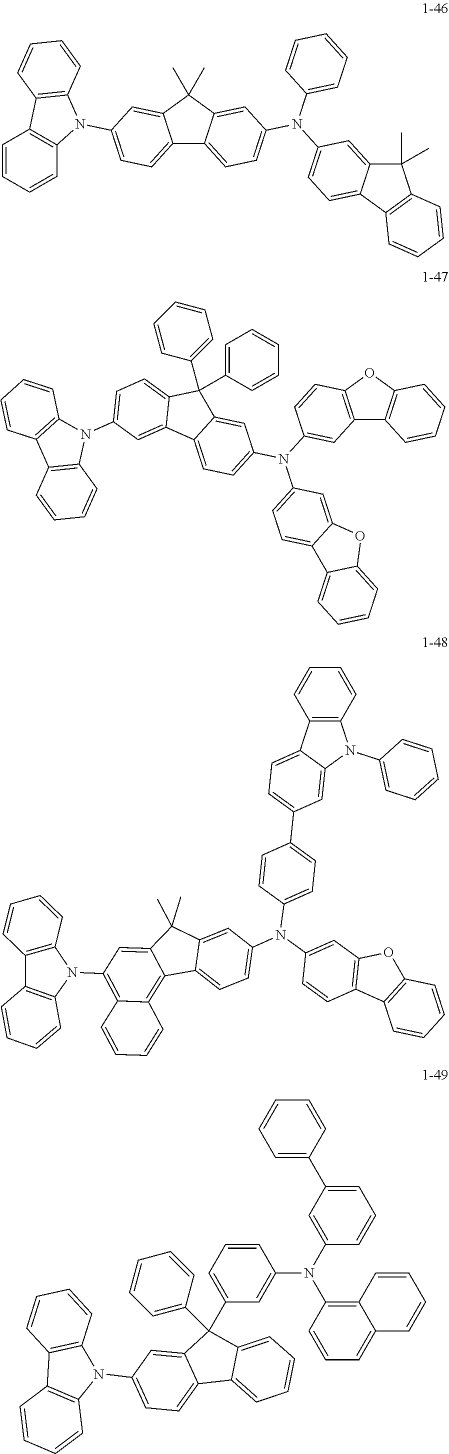 [SMILES: CC1(C)c2ccccc2-c2ccc(N(c3ccccc3)c3ccc4c(c3)C(C)(C)c3cc(-n5c6ccccc6c6ccccc65)ccc3-4)cc21 ; c1ccc(C2(c3ccccc3)c3ccc(-n4c5ccccc5c5ccccc54)cc3-c3ccc(N(c4ccc5c(c4)oc4ccccc45)c4ccc5oc6ccccc6c5c4)cc32)cc1 ; CC1(C)c2cc(N(c3ccc(-c4ccc5c6ccccc6n(-c6ccccc6)c5c4)cc3)c3ccc4c(c3)oc3ccccc34)ccc2-c2c1cc(-n1c3ccccc3c3ccccc31)c1ccccc21 ; c1ccc(-c2cccc(N(c3cccc(C4(c5ccccc5)c5ccccc5-c5ccc(-n6c7ccccc7c7ccccc76)cc54)c3)c3cccc4ccccc34)c2)cc1]

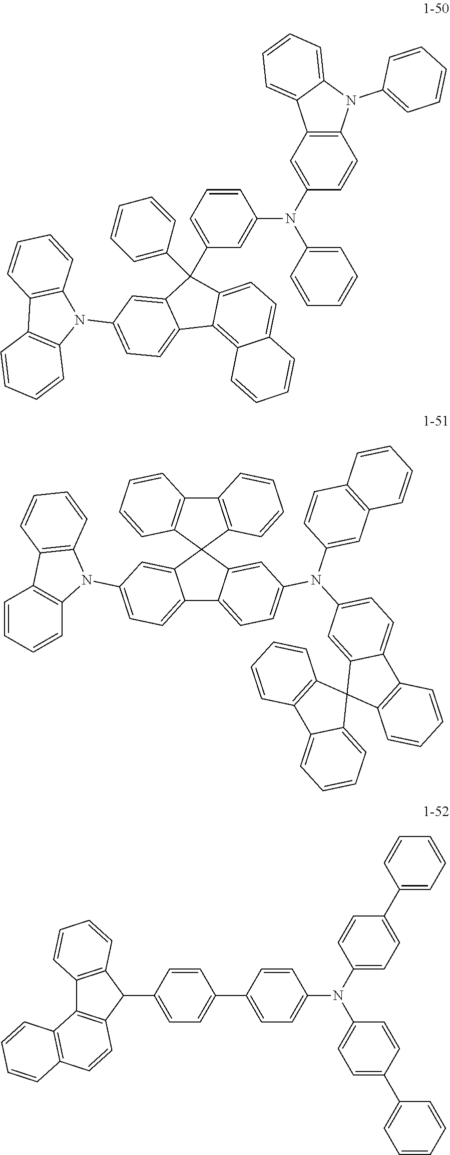 [SMILES: c1ccc(N(c2cccc(C3(c4ccccc4)c4cc(-n5c6ccccc6c6ccccc65)ccc4-c4c3ccc3ccccc43)c2)c2ccc3c(c2)c2ccccc2n3-c2ccccc2)cc1 ; c1ccc2c(c1)-c1ccccc1C21c2ccccc2-c2ccc(N(c3ccc4c(c3)C3(c5ccccc5-c5ccccc53)c3cc(-n5c6ccccc6c6ccccc65)ccc3-4)c3ccc4ccccc4c3)cc21 ; c1ccc(-c2ccc(N(c3ccc(-c4ccccc4)cc3)c3ccc(-c4ccc(C5c6ccccc6-c6c5ccc5ccccc65)cc4)cc3)cc2)cc1]

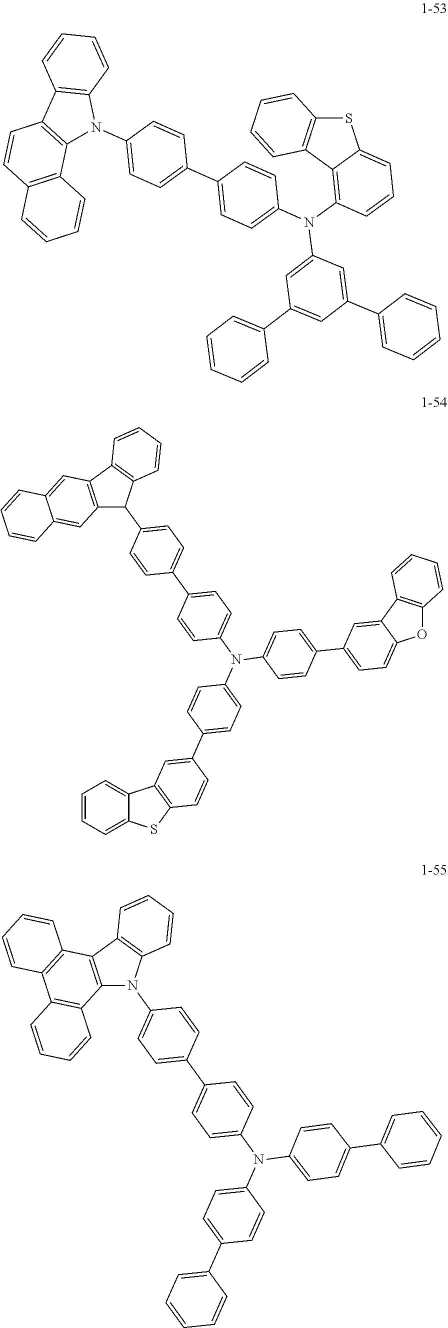 [SMILES: c1ccc(-c2cc(-c3ccccc3)cc(N(c3ccc(-c4ccc(-n5c6ccccc6c6ccc7ccccc7c65)cc4)cc3)c3cccc4sc5ccccc5c34)c2)cc1 ; c1ccc2c(c1)-c1cc3ccccc3cc1C2c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4ccccc4)cc3)c3ccc(-c4ccc(-n5c6ccccc6c6c7ccccc7c7ccccc7c65)cc4)cc3)cc2)cc1]

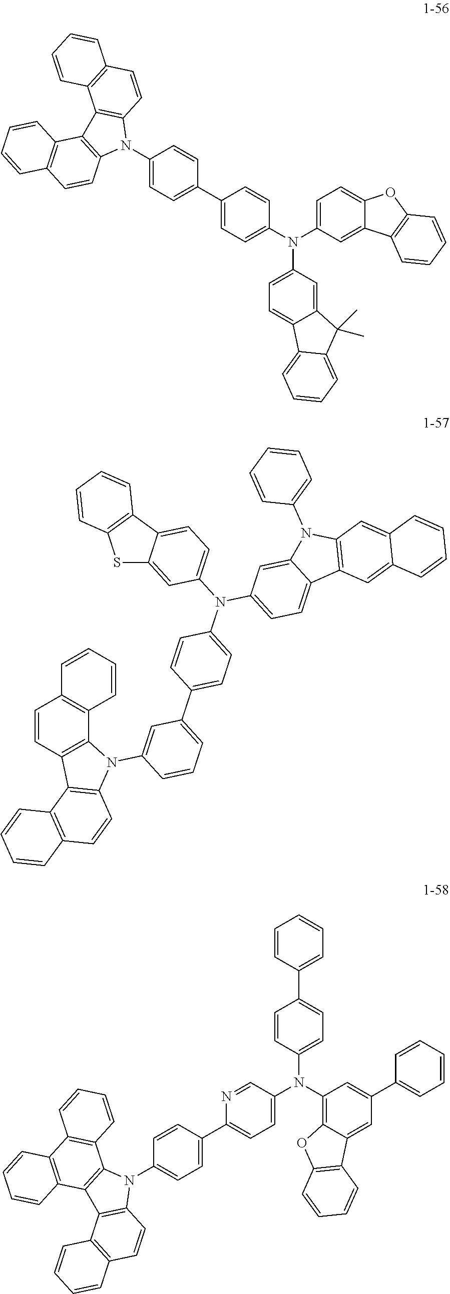 [SMILES: CC1(C)c2ccccc2-c2ccc(N(c3ccc(-c4ccc(-n5c6ccc7ccccc7c6c6c7ccccc7ccc65)cc4)cc3)c3ccc4oc5ccccc5c4c3)cc21 ; c1ccc(-n2c3cc(N(c4ccc(-c5cccc(-n6c7ccc8ccccc8c7c7ccc8ccccc8c76)c5)cc4)c4ccc5c(c4)sc4ccccc45)ccc3c3cc4ccccc4cc32)cc1 ; c1ccc(-c2ccc(N(c3ccc(-c4ccc(-n5c6ccc7ccccc7c6c6c7ccccc7c7ccccc7c65)cc4)nc3)c3cc(-c4ccccc4)cc4c3oc3ccccc34)cc2)cc1]

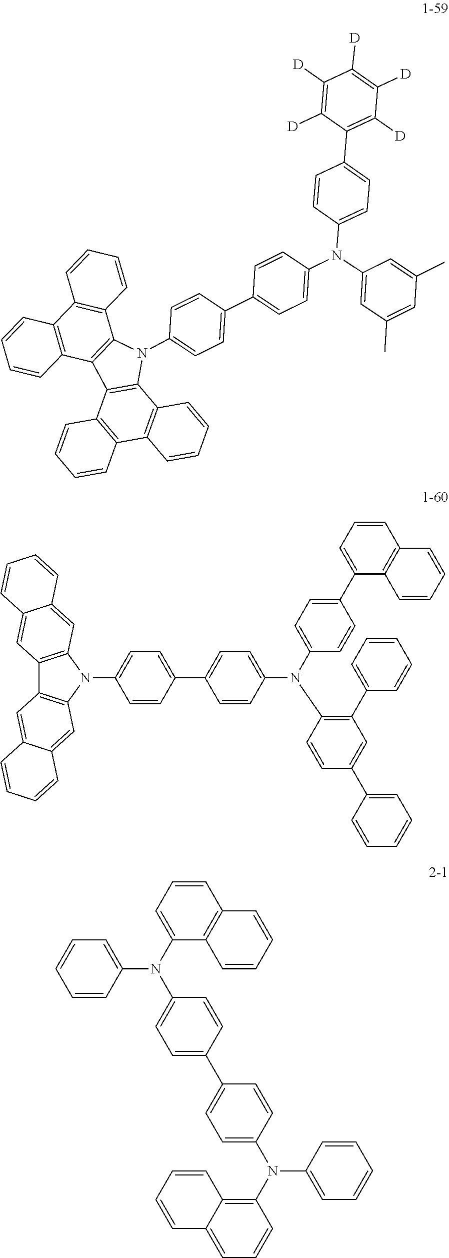 [SMILES: [2H]c1c([2H])c([2H])c(-c2ccc(N(c3ccc(-c4ccc(-n5c6c7ccccc7c7ccccc7c6c6c7ccccc7c7ccccc7c65)cc4)cc3)c3cc(C)cc(C)c3)cc2)c([2H])c1[2H] ; c1ccc(-c2ccc(N(c3ccc(-c4ccc(-n5c6cc7ccccc7cc6c6cc7ccccc7cc65)cc4)cc3)c3ccc(-c4cccc5ccccc45)cc3)c(-c3ccccc3)c2)cc1 ; c1ccc(N(c2ccc(-c3ccc(N(c4ccccc4)c4cccc5ccccc45)cc3)cc2)c2cccc3ccccc23)cc1]

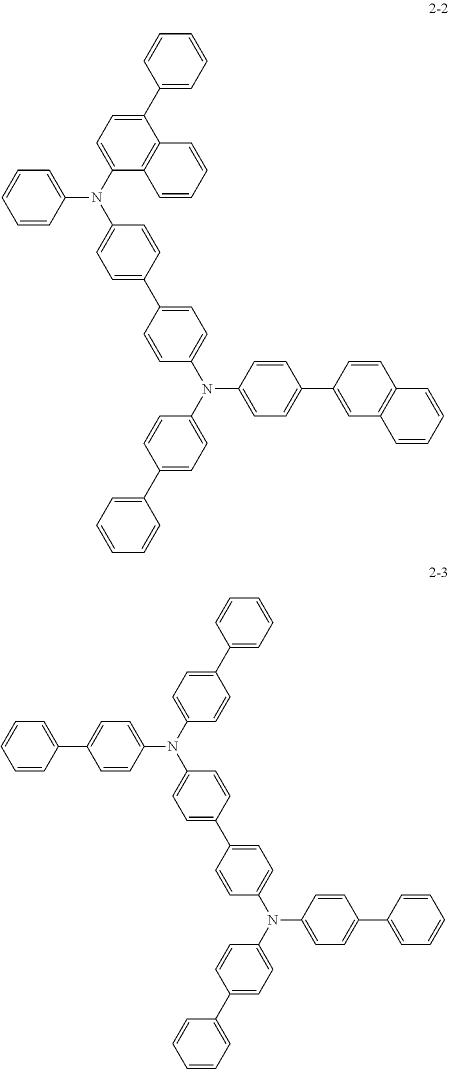 [SMILES: c1ccc(-c2ccc(N(c3ccc(-c4ccc(N(c5ccccc5)c5ccc(-c6ccccc6)c6ccccc56)cc4)cc3)c3ccc(-c4ccc5ccccc5c4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

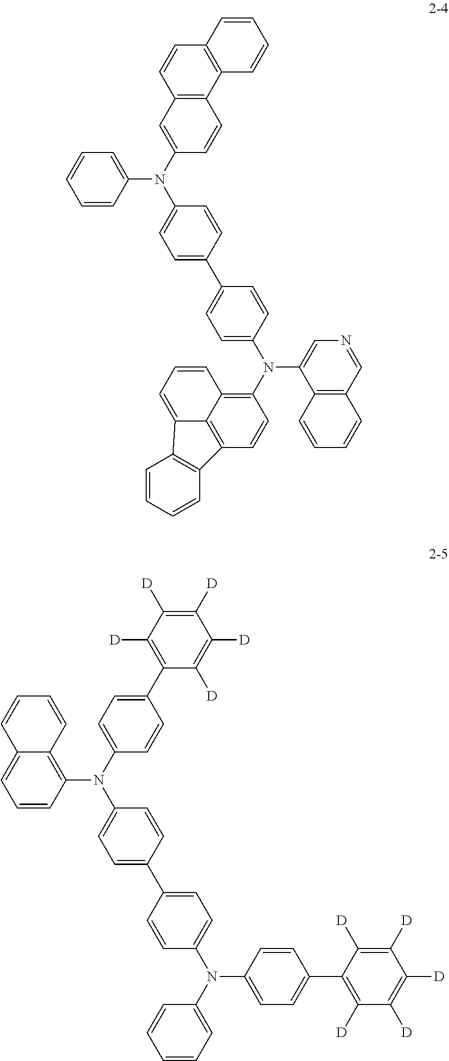 [SMILES: c1ccc(N(c2ccc(-c3ccc(N(c4cncc5ccccc45)c4ccc5c6c(cccc46)-c4ccccc4-5)cc3)cc2)c2ccc3c(ccc4ccccc43)c2)cc1 ; [2H]c1c([2H])c([2H])c(-c2ccc(N(c3ccccc3)c3ccc(-c4ccc(N(c5ccc(-c6c([2H])c([2H])c([2H])c([2H])c6[2H])cc5)c5cccc6ccccc56)cc4)cc3)cc2)c([2H])c1[2H]]

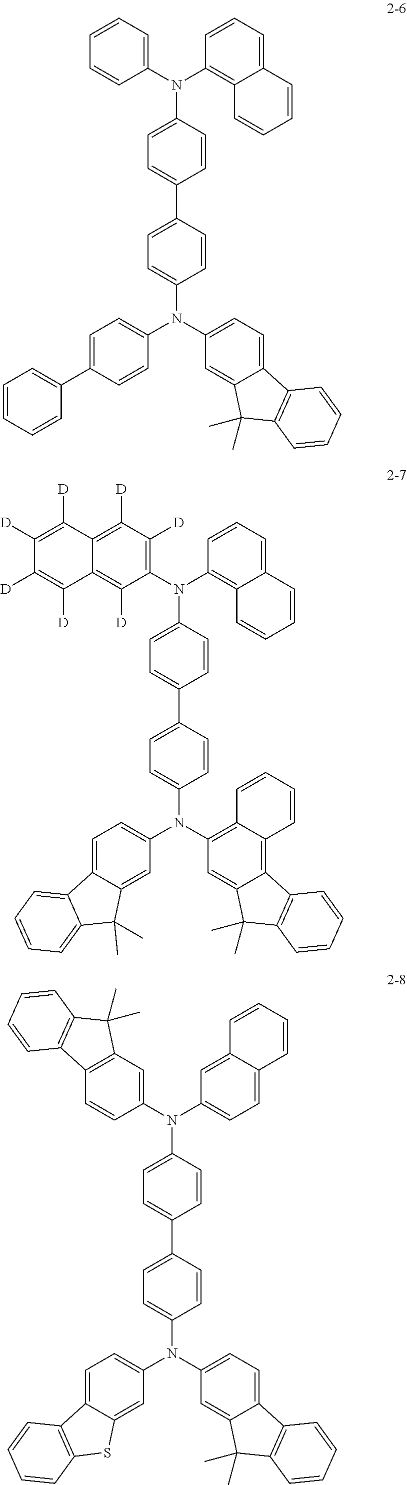

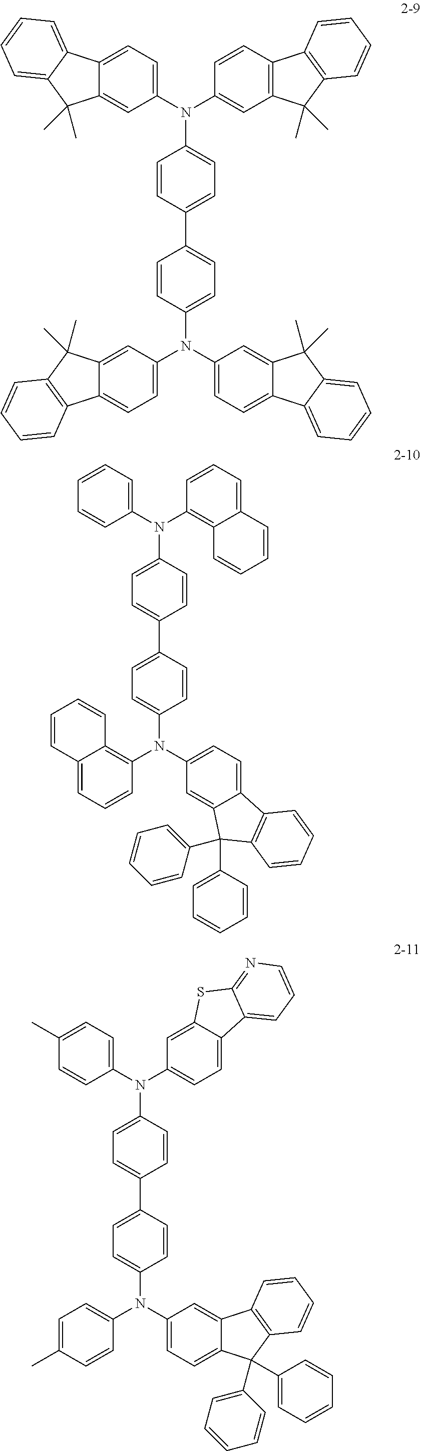

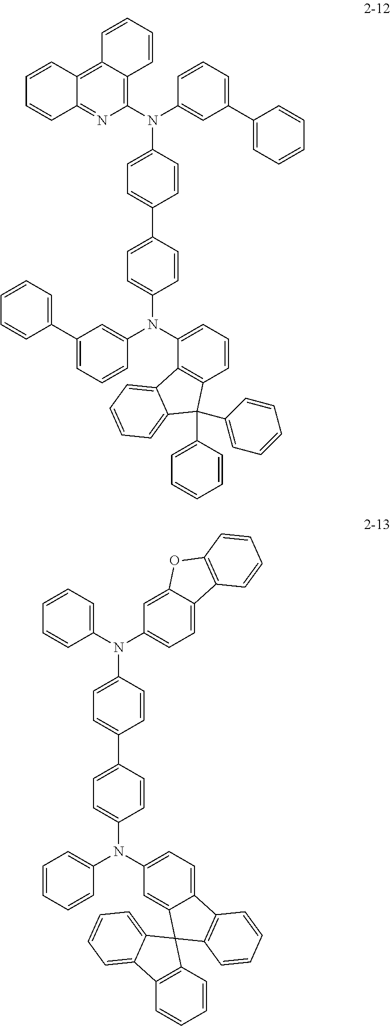 [SMILES: c1ccc(-c2cccc(N(c3ccc(-c4ccc(N(c5cccc(-c6ccccc6)c5)c5nc6ccccc6c6ccccc56)cc4)cc3)c3cccc4c3-c3ccccc3C4(c3ccccc3)c3ccccc3)c2)cc1 ; c1ccc(N(c2ccc(-c3ccc(N(c4ccccc4)c4ccc5c(c4)oc4ccccc45)cc3)cc2)c2ccc3c(c2)C2(c4ccccc4-c4ccccc42)c2ccccc2-3)cc1]

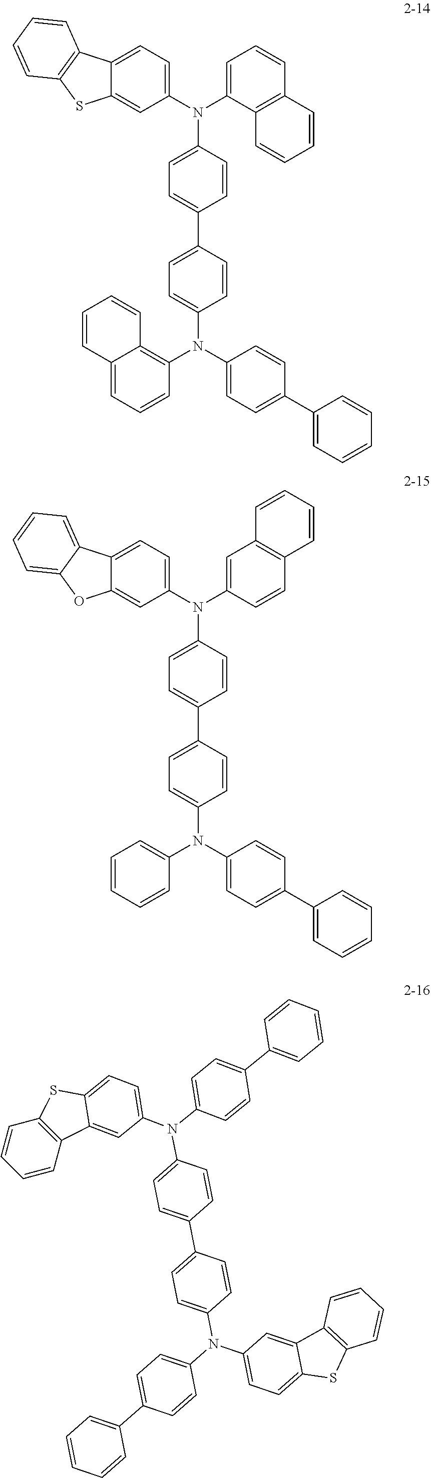 [SMILES: c1ccc(-c2ccc(N(c3ccc(-c4ccc(N(c5ccc6c(c5)sc5ccccc56)c5cccc6ccccc56)cc4)cc3)c3cccc4ccccc34)cc2)cc1 ; c1ccc(-c2ccc(N(c3ccccc3)c3ccc(-c4ccc(N(c5ccc6ccccc6c5)c5ccc6c(c5)oc5ccccc56)cc4)cc3)cc2)cc1 ; c1ccc(-c2ccc(N(c3ccc(-c4ccc(N(c5ccc(-c6ccccc6)cc5)c5ccc6sc7ccccc7c6c5)cc4)cc3)c3ccc4sc5ccccc5c4c3)cc2)cc1]

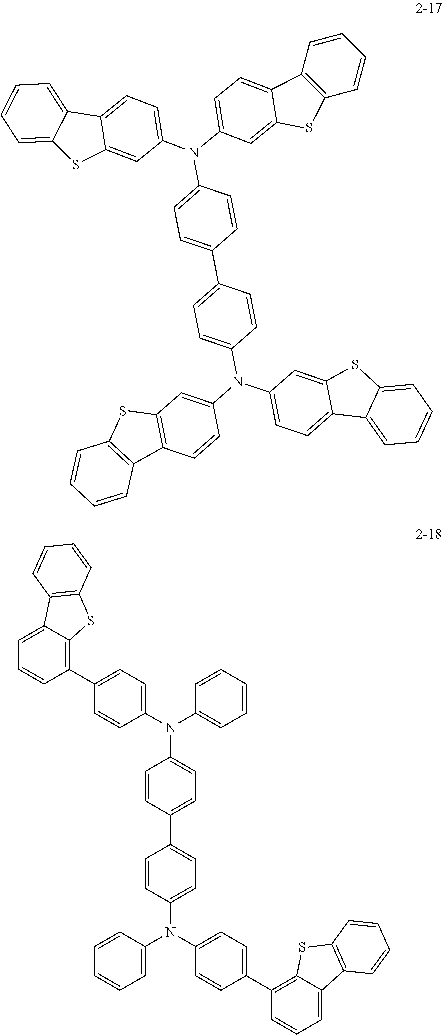

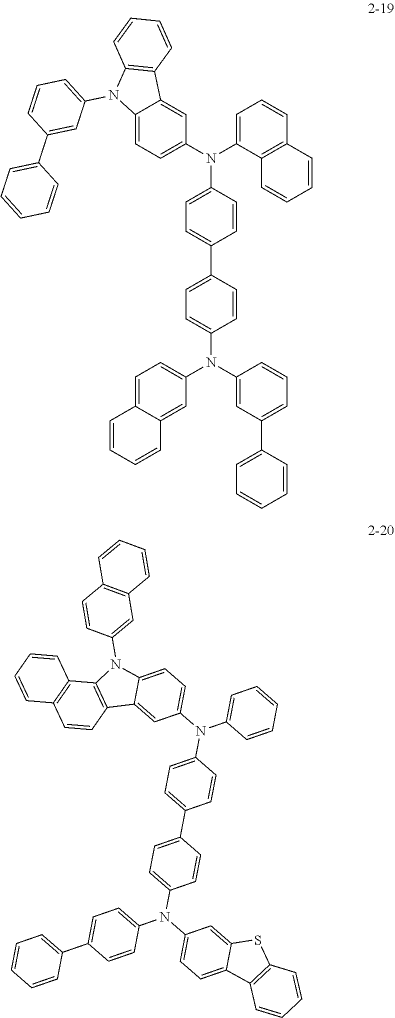 [SMILES: c1ccc(-c2cccc(N(c3ccc(-c4ccc(N(c5ccc6c(c5)c5ccccc5n6-c5cccc(-c6ccccc6)c5)c5cccc6ccccc56)cc4)cc3)c3ccc4ccccc4c3)c2)cc1 ; c1ccc(-c2ccc(N(c3ccc(-c4ccc(N(c5ccccc5)c5ccc6c(c5)c5ccc7ccccc7c5n6-c5ccc6ccccc6c5)cc4)cc3)c3ccc4c(c3)sc3ccccc34)cc2)cc1]

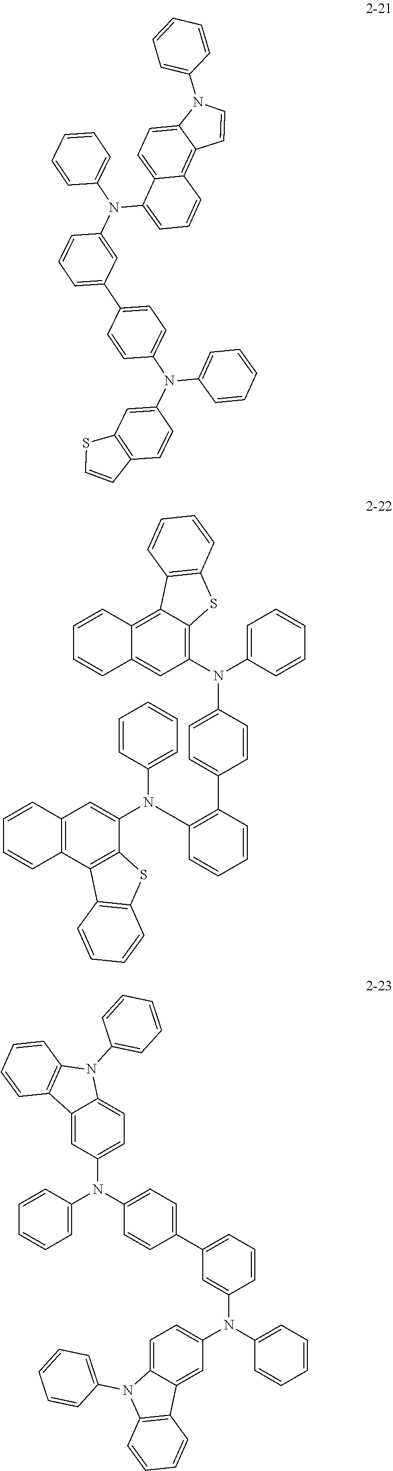

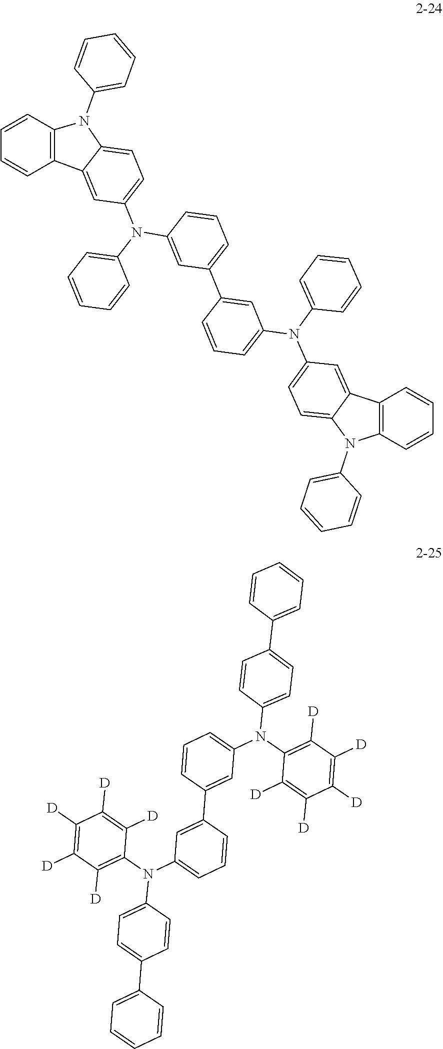 [SMILES: c1ccc(N(c2cccc(-c3cccc(N(c4ccccc4)c4ccc5c(c4)c4ccccc4n5-c4ccccc4)c3)c2)c2ccc3c(c2)c2ccccc2n3-c2ccccc2)cc1 ; [2H]c1c([2H])c([2H])c(N(c2ccc(-c3ccccc3)cc2)c2cccc(-c3cccc(N(c4ccc(-c5ccccc5)cc4)c4c([2H])c([2H])c([2H])c([2H])c4[2H])c3)c2)c([2H])c1[2H]]

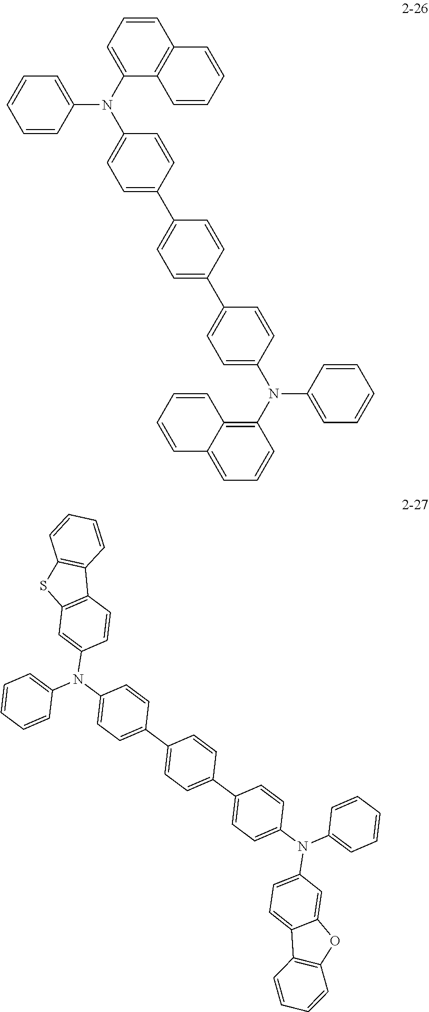 [SMILES: c1ccc(N(c2ccc(-c3ccc(-c4ccc(N(c5ccccc5)c5cccc6ccccc56)cc4)cc3)cc2)c2cccc3ccccc23)cc1 ; c1ccc(N(c2ccc(-c3ccc(-c4ccc(N(c5ccccc5)c5ccc6c(c5)sc5ccccc56)cc4)cc3)cc2)c2ccc3c(c2)oc2ccccc23)cc1]

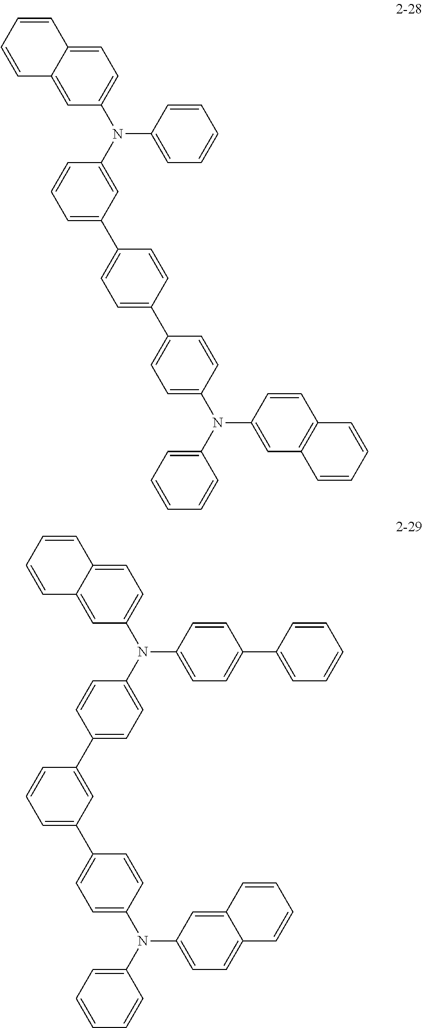 [SMILES: c1ccc(N(c2ccc(-c3ccc(-c4cccc(N(c5ccccc5)c5ccc6ccccc6c5)c4)cc3)cc2)c2ccc3ccccc3c2)cc1 ; c1ccc(-c2ccc(N(c3ccc(-c4cccc(-c5ccc(N(c6ccccc6)c6ccc7ccccc7c6)cc5)c4)cc3)c3ccc4ccccc4c3)cc2)cc1]

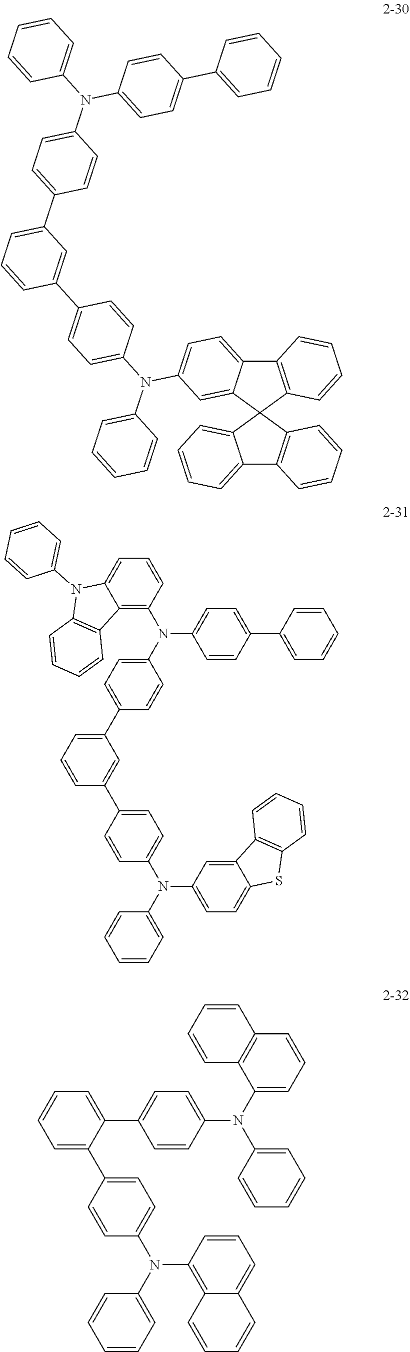 [SMILES: c1ccc(-c2ccc(N(c3ccccc3)c3ccc(-c4cccc(-c5ccc(N(c6ccccc6)c6ccc7c(c6)C6(c8ccccc8-c8ccccc86)c6ccccc6-7)cc5)c4)cc3)cc2)cc1 ; c1ccc(-c2ccc(N(c3ccc(-c4cccc(-c5ccc(N(c6ccccc6)c6ccc7sc8ccccc8c7c6)cc5)c4)cc3)c3cccc4c3c3ccccc3n4-c3ccccc3)cc2)cc1 ; c1ccc(N(c2ccc(-c3ccccc3-c3ccc(N(c4ccccc4)c4cccc5ccccc45)cc3)cc2)c2cccc3ccccc23)cc1]

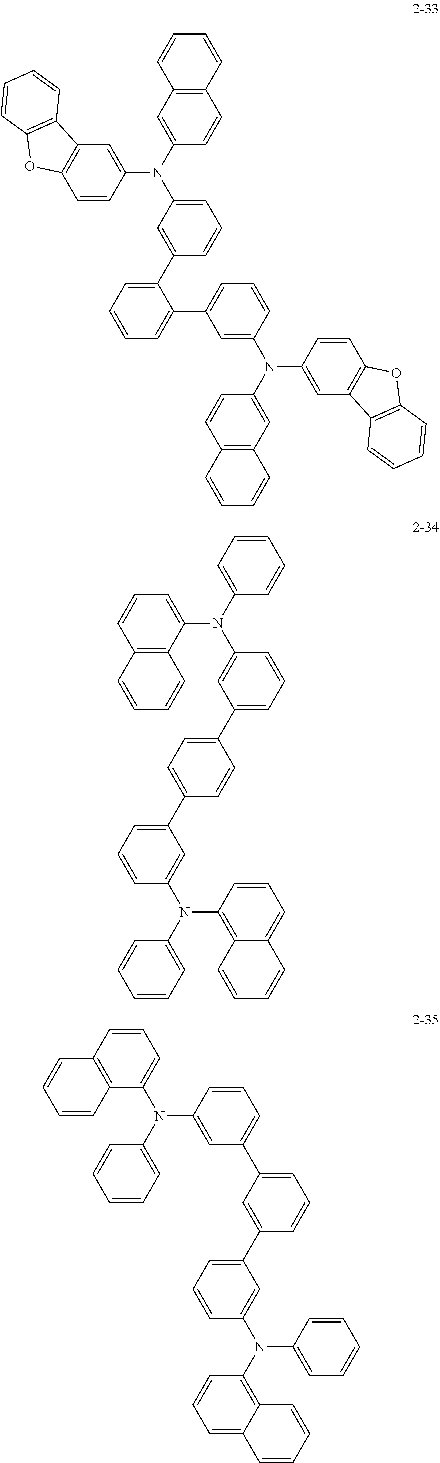

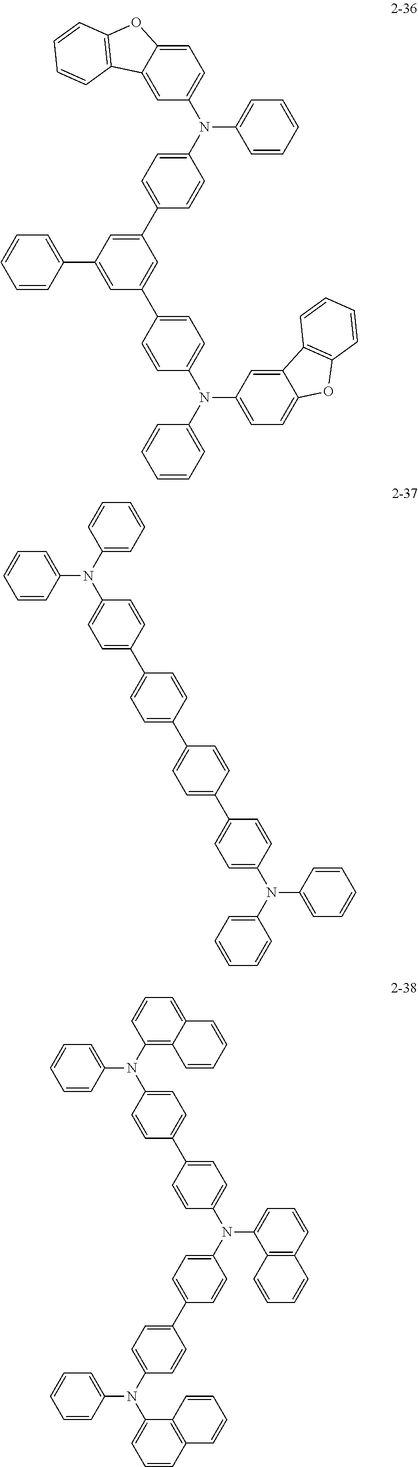

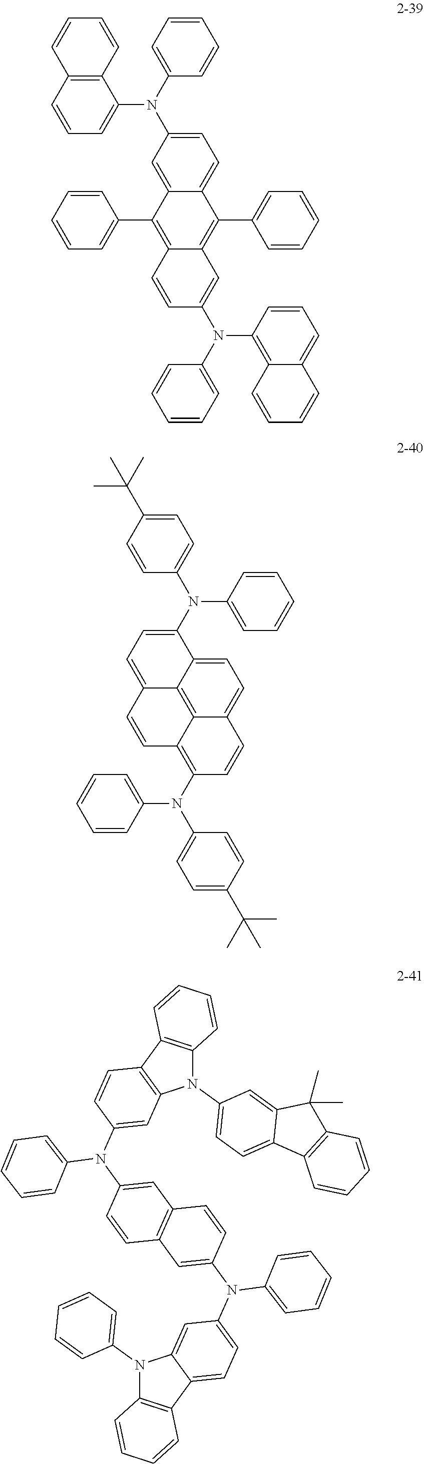

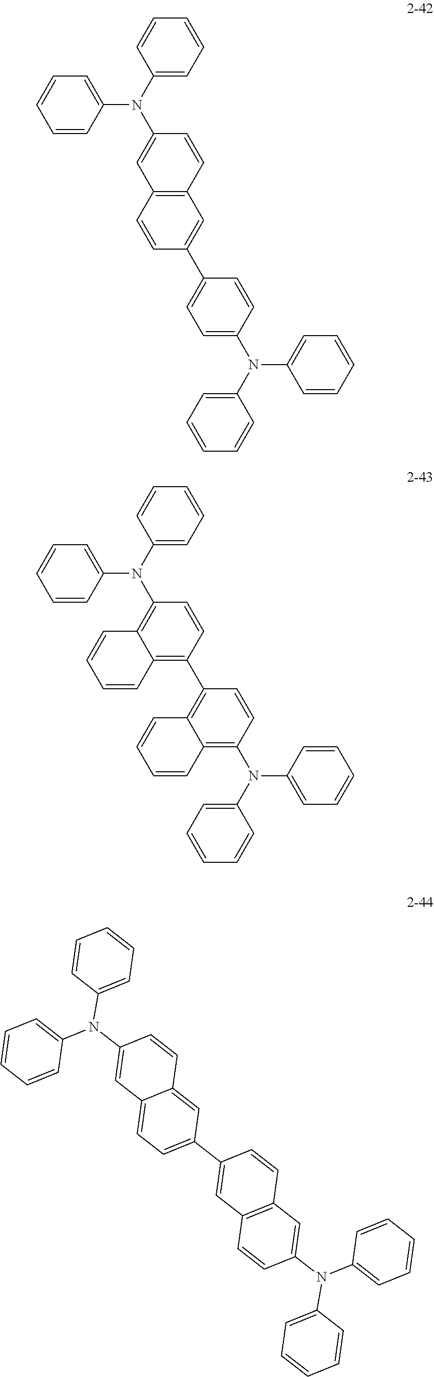

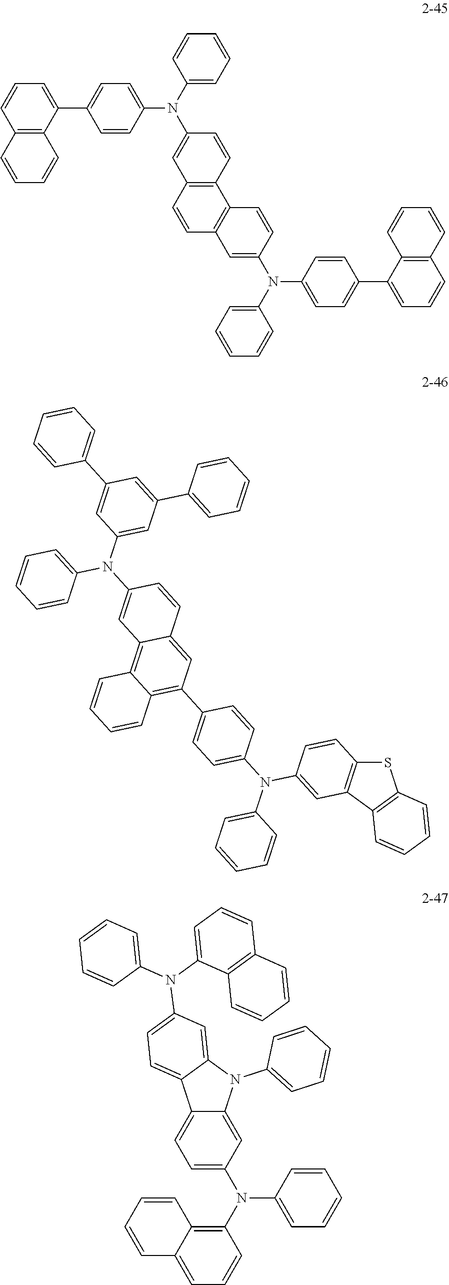

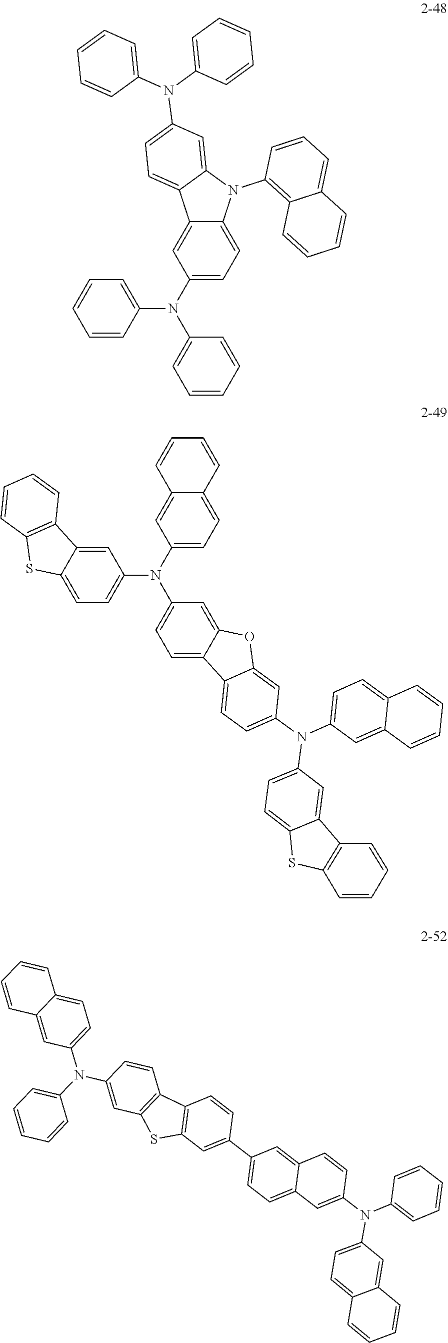 [SMILES: c1ccc(N(c2ccccc2)c2ccc3c(c2)c2ccc(N(c4ccccc4)c4ccccc4)cc2n3-c2cccc3ccccc23)cc1 ; c1ccc2cc(N(c3ccc4c(c3)oc3cc(N(c5ccc6ccccc6c5)c5ccc6sc7ccccc7c6c5)ccc34)c3ccc4sc5ccccc5c4c3)ccc2c1 ; c1ccc(N(c2ccc3ccccc3c2)c2ccc3cc(-c4ccc5c(c4)sc4cc(N(c6ccccc6)c6ccc7ccccc7c6)ccc45)ccc3c2)cc1]

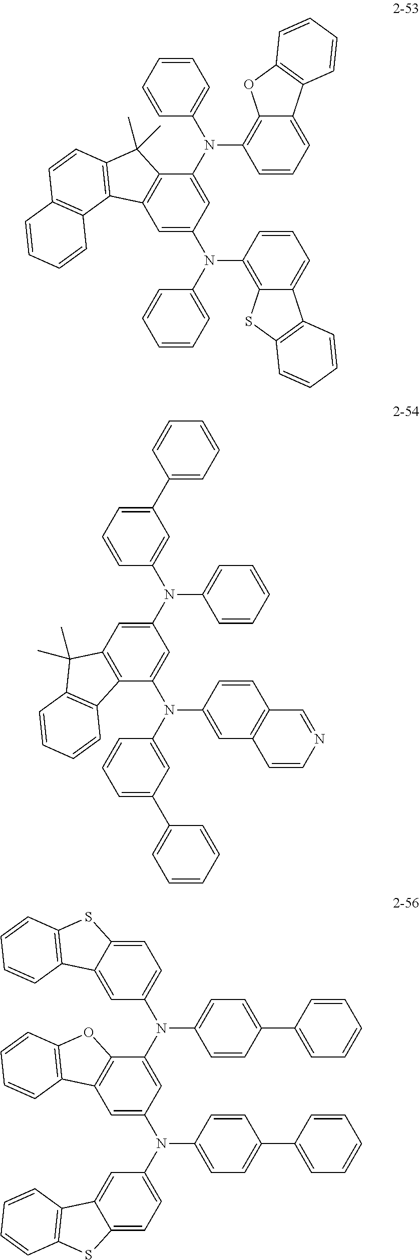 [SMILES: CC1(C)c2ccc3ccccc3c2-c2cc(N(c3ccccc3)c3cccc4c3sc3ccccc34)cc(N(c3ccccc3)c3cccc4c3oc3ccccc34)c21 ; CC1(C)c2ccccc2-c2c(N(c3cccc(-c4ccccc4)c3)c3ccc4cnccc4c3)cc(N(c3ccccc3)c3cccc(-c4ccccc4)c3)cc21 ; c1ccc(-c2ccc(N(c3ccc4sc5ccccc5c4c3)c3cc(N(c4ccc(-c5ccccc5)cc4)c4ccc5sc6ccccc6c5c4)c4oc5ccccc5c4c3)cc2)cc1]

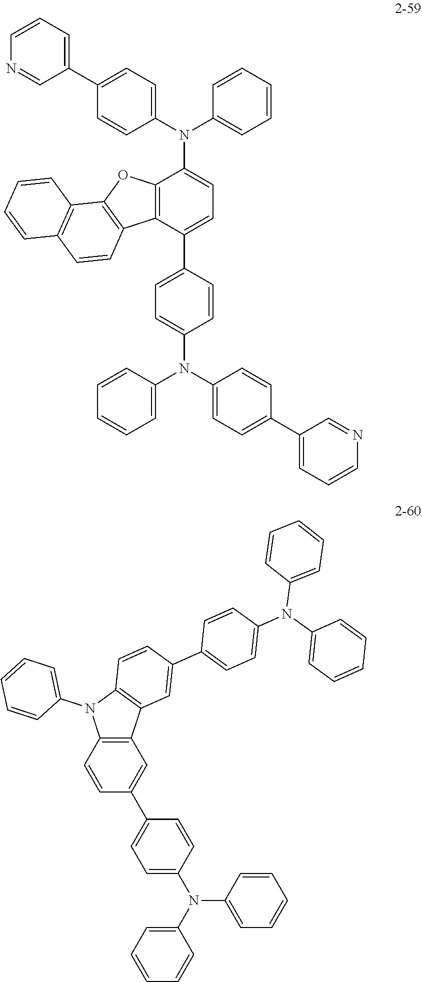

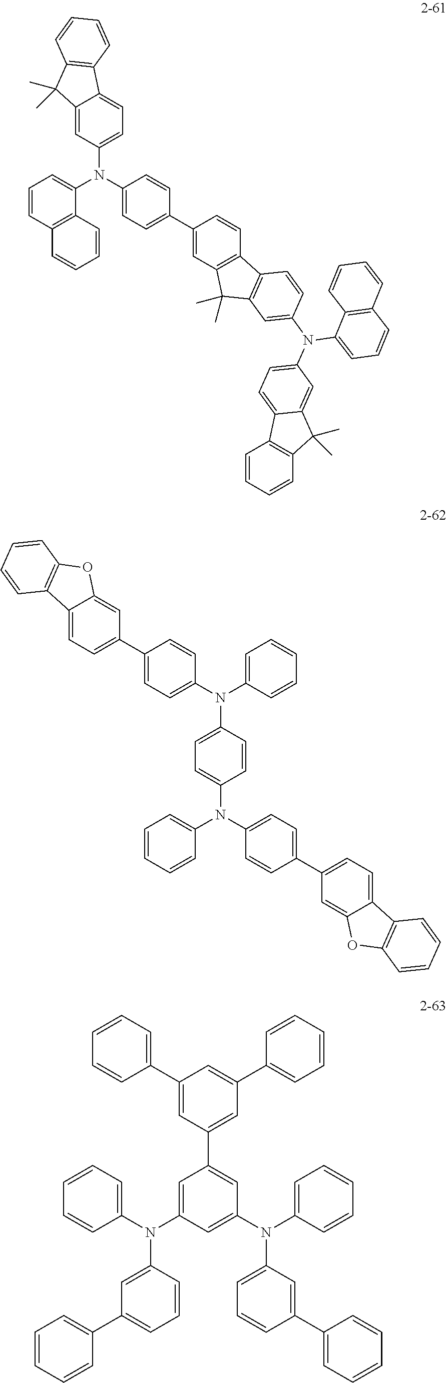 [SMILES: CC1(C)c2ccccc2-c2ccc(N(c3ccc(-c4ccc5c(c4)C(C)(C)c4cc(N(c6ccc7c(c6)C(C)(C)c6ccccc6-7)c6cccc7ccccc67)ccc4-5)cc3)c3cccc4ccccc34)cc21 ; c1ccc(N(c2ccc(-c3ccc4c(c3)oc3ccccc34)cc2)c2ccc(N(c3ccccc3)c3ccc(-c4ccc5c(c4)oc4ccccc45)cc3)cc2)cc1 ; c1ccc(-c2cc(-c3ccccc3)cc(-c3cc(N(c4ccccc4)c4cccc(-c5ccccc5)c4)cc(N(c4ccccc4)c4cccc(-c5ccccc5)c4)c3)c2)cc1]

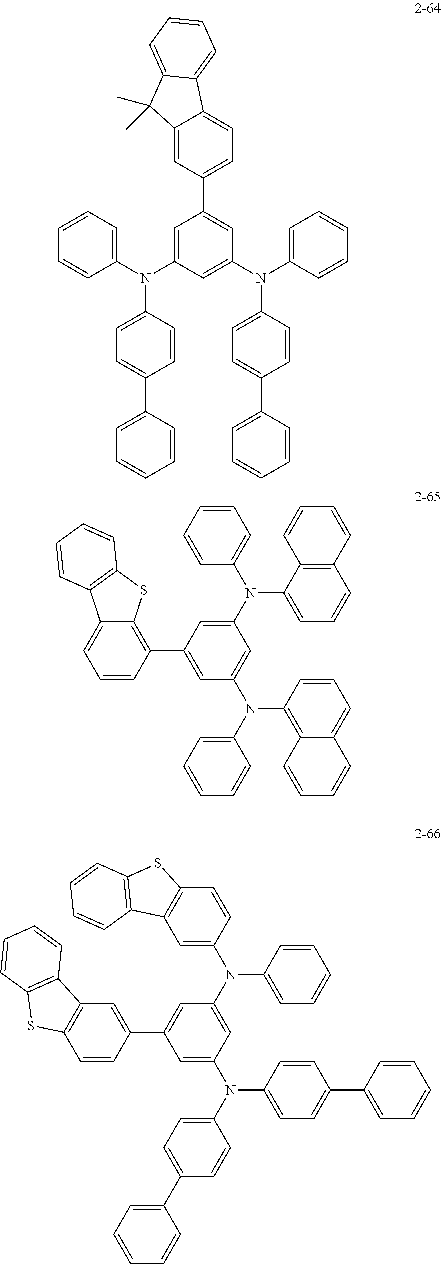

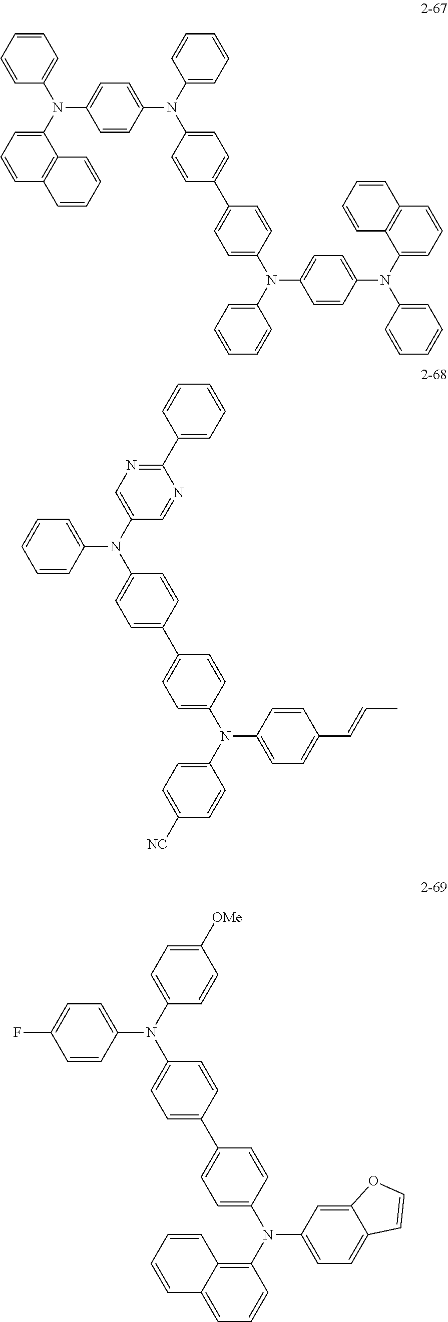

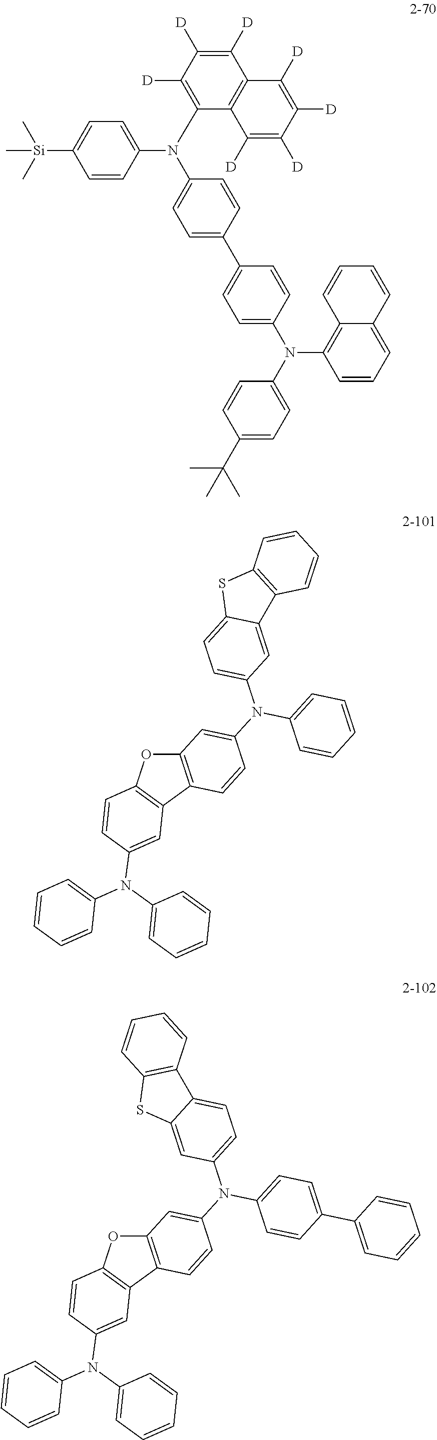 [SMILES: [2H]c1c([2H])c([2H])c2c(N(c3ccc(-c4ccc(N(c5ccc(C(C)(C)C)cc5)c5cccc6ccccc56)cc4)cc3)c3ccc([Si](C)(C)C)cc3)c([2H])c([2H])c([2H])c2c1[2H] ; c1ccc(N(c2ccccc2)c2ccc3oc4cc(N(c5ccccc5)c5ccc6sc7ccccc7c6c5)ccc4c3c2)cc1 ; c1ccc(-c2ccc(N(c3ccc4c(c3)oc3ccc(N(c5ccccc5)c5ccccc5)cc34)c3ccc4c(c3)sc3ccccc34)cc2)cc1]

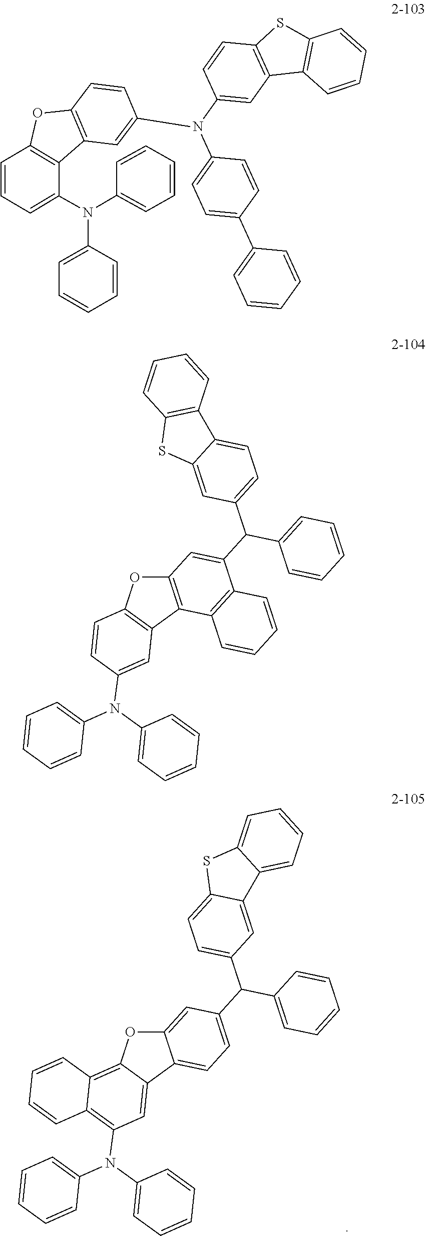 [SMILES: c1ccc(-c2ccc(N(c3ccc4sc5ccccc5c4c3)c3ccc4oc5cccc(N(c6ccccc6)c6ccccc6)c5c4c3)cc2)cc1 ; c1ccc(C(c2ccc3c(c2)sc2ccccc23)c2cc3oc4ccc(N(c5ccccc5)c5ccccc5)cc4c3c3ccccc23)cc1 ; c1ccc(C(c2ccc3c(c2)oc2c4ccccc4c(N(c4ccccc4)c4ccccc4)cc32)c2ccc3sc4ccccc4c3c2)cc1]

12. The organic electric element according to claim 1, wherein the second host compound represented by Formula (2) comprises any one of the following Compounds 3-1 to 3-124:

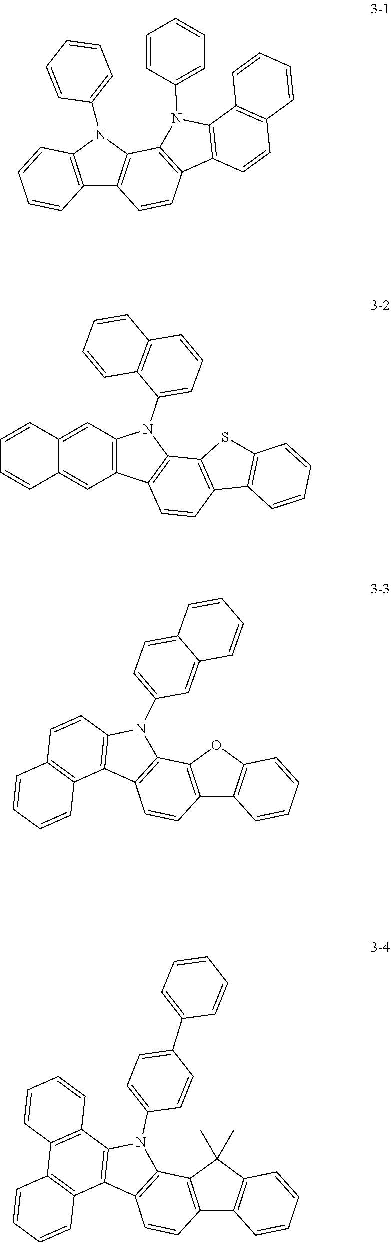 [SMILES: c1ccc(-n2c3ccccc3c3ccc4c5ccc6ccccc6c5n(-c5ccccc5)c4c32)cc1 ; c1ccc2cc3c(cc2c1)c1ccc2c4ccccc4sc2c1n3-c1cccc2ccccc12 ; c1ccc2cc(-n3c4ccc5ccccc5c4c4ccc5c6ccccc6oc5c43)ccc2c1 ; CC1(C)c2ccccc2-c2ccc3c4c5ccccc5c5ccccc5c4n(-c4ccc(-c5ccccc5)cc4)c3c21]

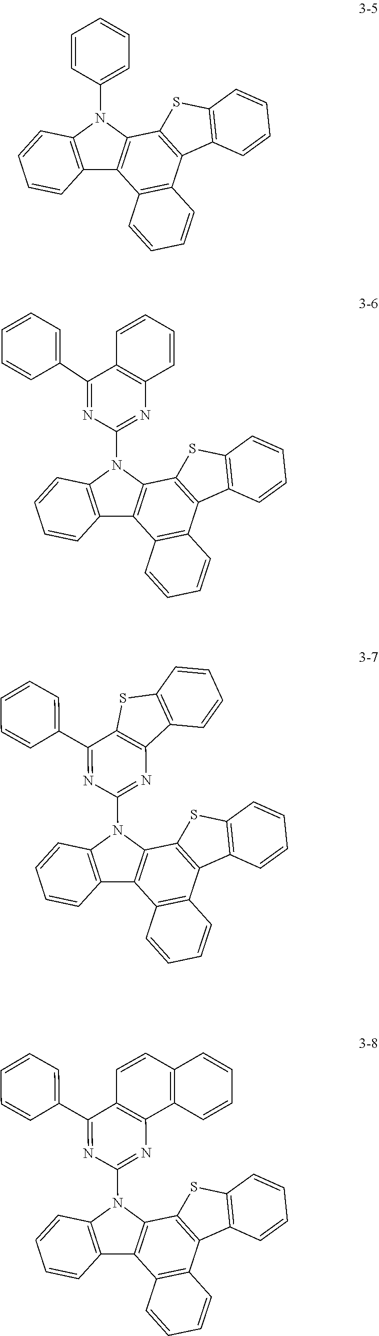 [SMILES: c1ccc(-n2c3ccccc3c3c4ccccc4c4c5ccccc5sc4c32)cc1 ; c1ccc(-c2nc(-n3c4ccccc4c4c5ccccc5c5c6ccccc6sc5c43)nc3ccccc23)cc1 ; c1ccc(-c2nc(-n3c4ccccc4c4c5ccccc5c5c6ccccc6sc5c43)nc3c2sc2ccccc23)cc1 ; c1ccc(-c2nc(-n3c4ccccc4c4c5ccccc5c5c6ccccc6sc5c43)nc3c2ccc2ccccc23)cc1]

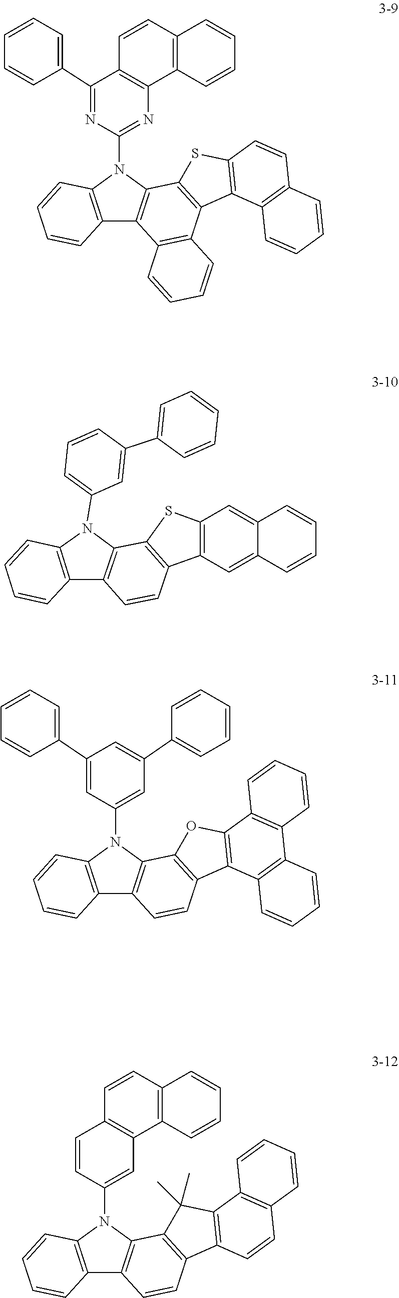 [SMILES: c1ccc(-c2nc(-n3c4ccccc4c4c5ccccc5c5c(sc6ccc7ccccc7c65)c43)nc3c2ccc2ccccc23)cc1 ; c1ccc(-c2cccc(-n3c4ccccc4c4ccc5c6cc7ccccc7cc6sc5c43)c2)cc1 ; c1ccc(-c2cc(-c3ccccc3)cc(-n3c4ccccc4c4ccc5c(oc6c7ccccc7c7ccccc7c56)c43)c2)cc1 ; CC1(C)c2c(ccc3ccccc23)-c2ccc3c4ccccc4n(-c4ccc5ccc6ccccc6c5c4)c3c21]

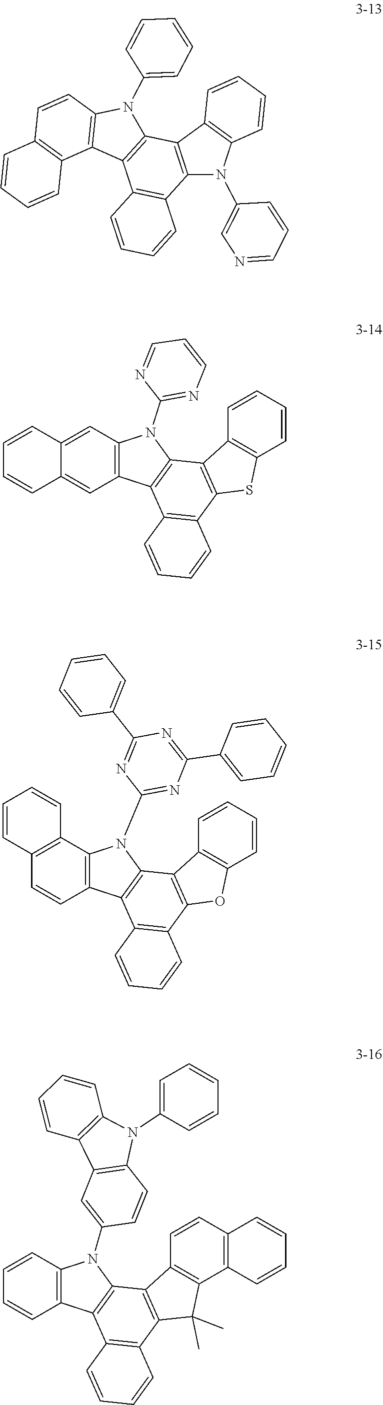 [SMILES: c1ccc(-n2c3ccc4ccccc4c3c3c4ccccc4c4c(c5ccccc5n4-c4cccnc4)c32)cc1 ; c1cnc(-n2c3cc4ccccc4cc3c3c4ccccc4c4sc5ccccc5c4c32)nc1 ; c1ccc(-c2nc(-c3ccccc3)nc(-n3c4c5ccccc5ccc4c4c5ccccc5c5oc6ccccc6c5c43)n2)cc1 ; CC1(C)c2c(ccc3ccccc23)-c2c1c1ccccc1c1c3ccccc3n(-c3ccc4c(c3)c3ccccc3n4-c3ccccc3)c21]

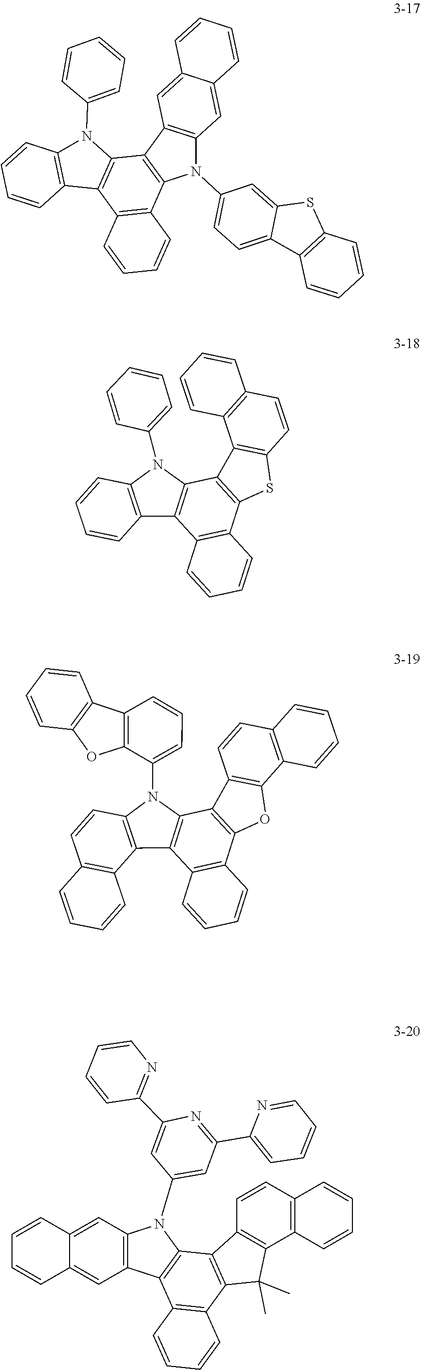 [SMILES: c1ccc(-n2c3ccccc3c3c4ccccc4c4c(c5cc6ccccc6cc5n4-c4ccc5c(c4)sc4ccccc45)c32)cc1 ; c1ccc(-n2c3ccccc3c3c4ccccc4c4sc5ccc6ccccc6c5c4c32)cc1 ; c1ccc2c(c1)ccc1c2oc2c3ccccc3c3c4c5ccccc5ccc4n(-c4cccc5c4oc4ccccc45)c3c12 ; CC1(C)c2c(ccc3ccccc23)-c2c1c1ccccc1c1c3cc4ccccc4cc3n(-c3cc(-c4ccccn4)nc(-c4ccccn4)c3)c21]

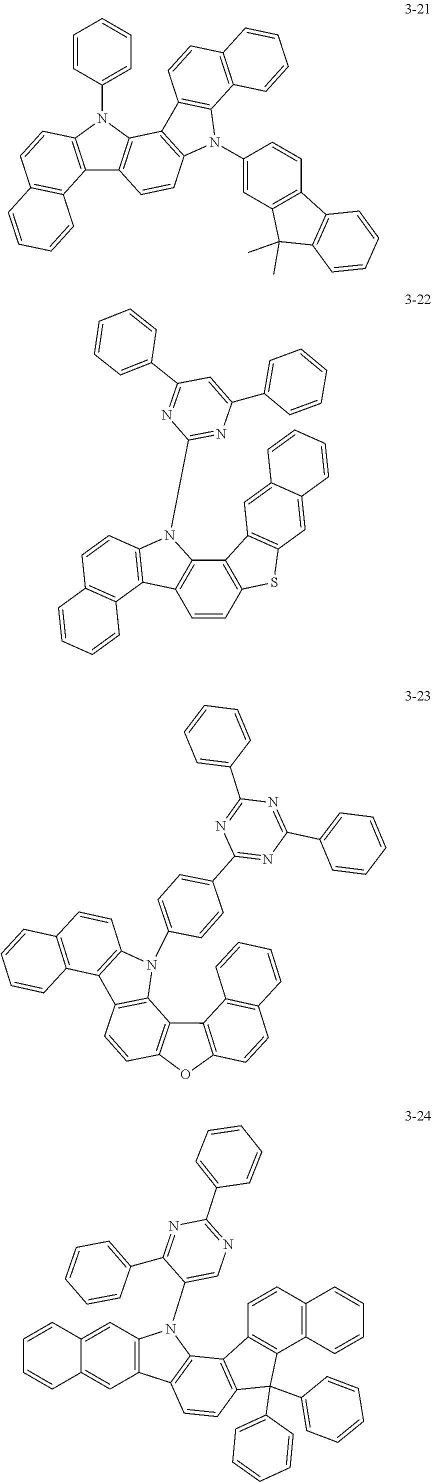 [SMILES: CC1(C)c2ccccc2-c2ccc(-n3c4ccc5c6c7ccccc7ccc6n(-c6ccccc6)c5c4c4ccc5ccccc5c43)cc21 ; c1ccc(-c2cc(-c3ccccc3)nc(-n3c4ccc5ccccc5c4c4ccc5sc6cc7ccccc7cc6c5c43)n2)cc1 ; c1ccc(-c2nc(-c3ccccc3)nc(-c3ccc(-n4c5ccc6ccccc6c5c5ccc6oc7ccc8ccccc8c7c6c54)cc3)n2)cc1 ; c1ccc(-c2ncc(-n3c4cc5ccccc5cc4c4ccc5c(c43)-c3ccc4ccccc4c3C5(c3ccccc3)c3ccccc3)c(-c3ccccc3)n2)cc1]

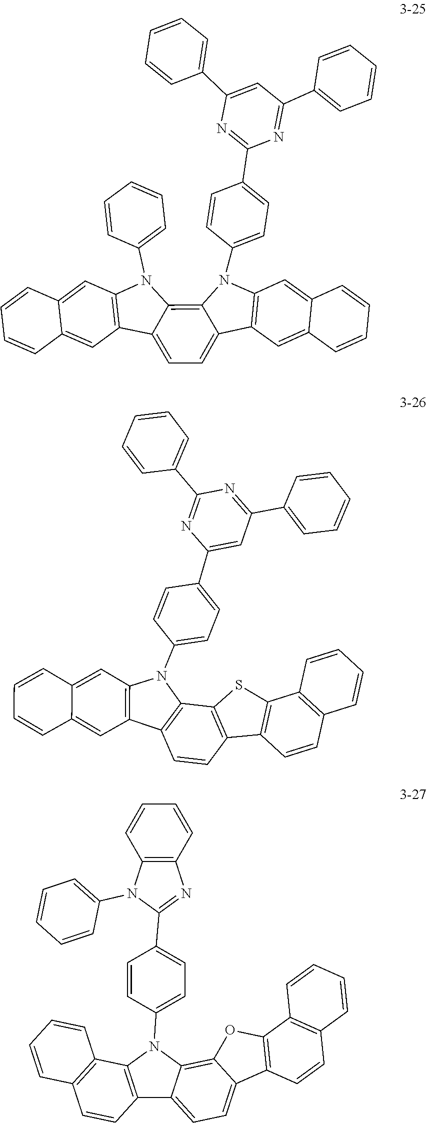

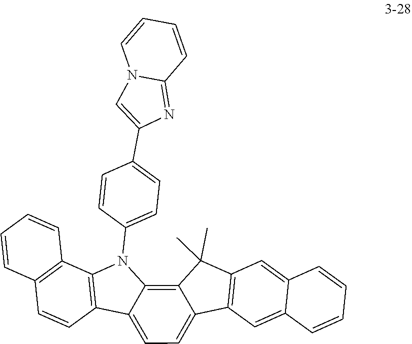 [SMILES: CC1(C)c2cc3ccccc3cc2-c2ccc3c4ccc5ccccc5c4n(-c4ccc(-c5cn6ccccc6n5)cc4)c3c21]

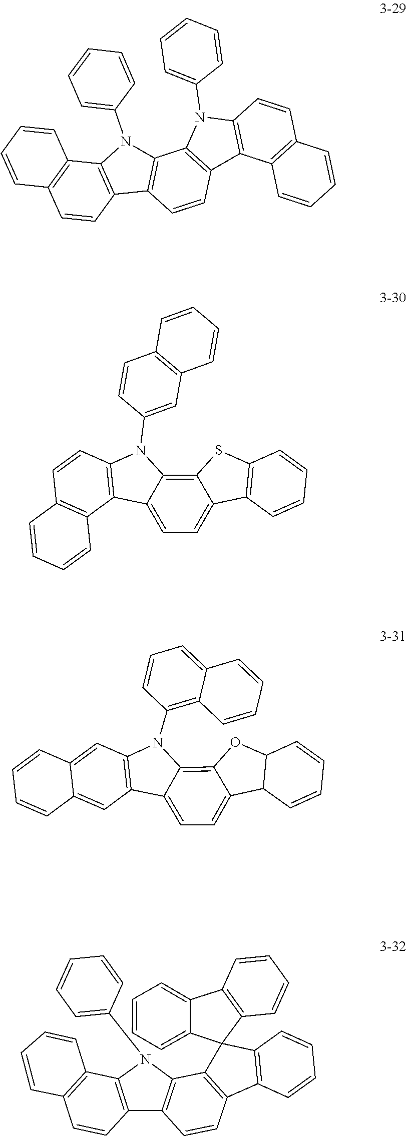 [SMILES: c1ccc(-n2c3ccc4ccccc4c3c3ccc4c5ccc6ccccc6c5n(-c5ccccc5)c4c32)cc1 ; c1ccc2cc(-n3c4ccc5ccccc5c4c4ccc5c6ccccc6sc5c43)ccc2c1 ; C1=CC2Oc3c(ccc4c5cc6ccccc6cc5n(-c5cccc6ccccc56)c34)C2C=C1 ; c1ccc(-n2c3c4c(ccc3c3ccc5ccccc5c32)-c2ccccc2C42c3ccccc3-c3ccccc32)cc1]

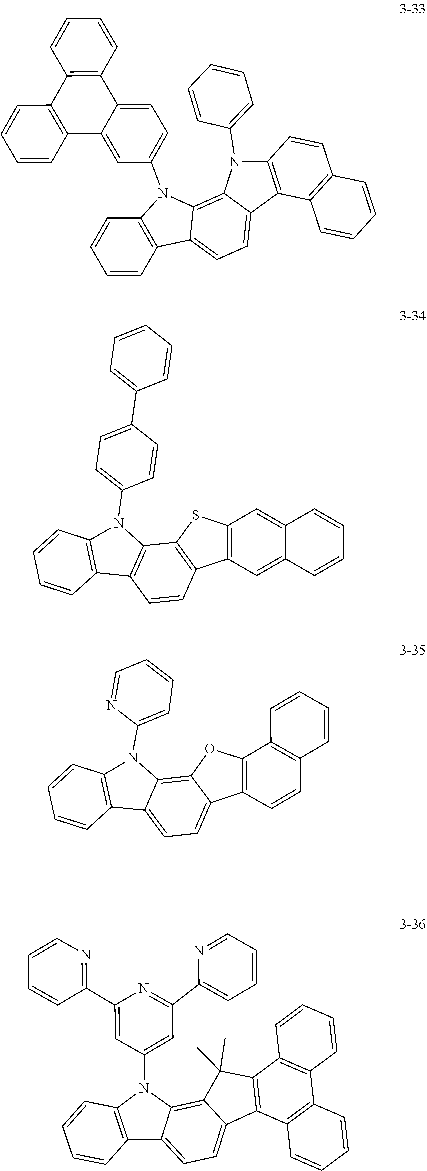 [SMILES: c1ccc(-n2c3ccc4ccccc4c3c3ccc4c5ccccc5n(-c5ccc6c7ccccc7c7ccccc7c6c5)c4c32)cc1 ; c1ccc(-c2ccc(-n3c4ccccc4c4ccc5c6cc7ccccc7cc6sc5c43)cc2)cc1 ; c1ccc(-n2c3ccccc3c3ccc4c5ccc6ccccc6c5oc4c32)nc1 ; CC1(C)c2c(c3ccccc3c3ccccc23)-c2ccc3c4ccccc4n(-c4cc(-c5ccccn5)nc(-c5ccccn5)c4)c3c21]

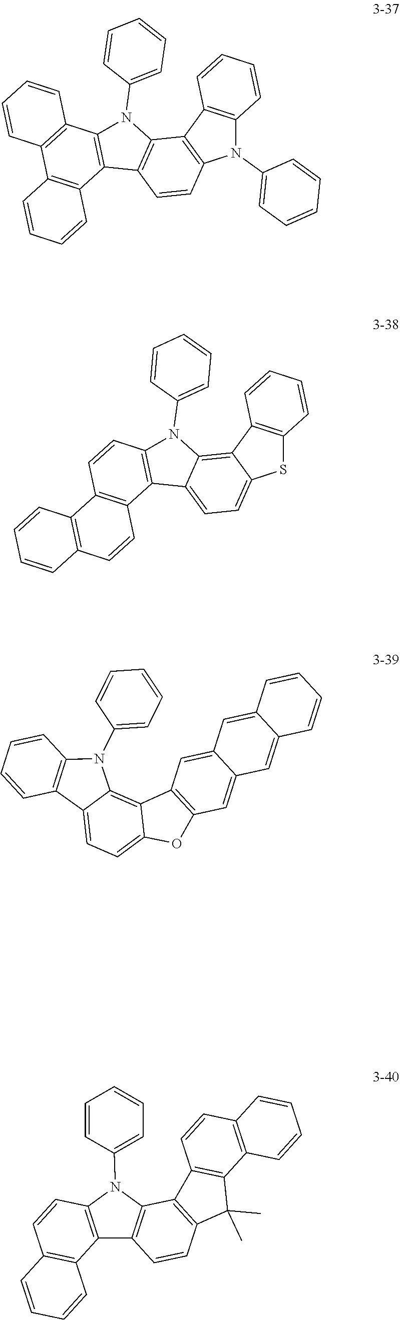 [SMILES: c1ccc(-n2c3ccccc3c3c2ccc2c4c5ccccc5c5ccccc5c4n(-c4ccccc4)c23)cc1 ; c1ccc(-n2c3ccc4c5ccccc5ccc4c3c3ccc4sc5ccccc5c4c32)cc1 ; c1ccc(-n2c3ccccc3c3ccc4oc5cc6cc7ccccc7cc6cc5c4c32)cc1 ; CC1(C)c2ccc3c4c5ccccc5ccc4n(-c4ccccc4)c3c2-c2ccc3ccccc3c21]

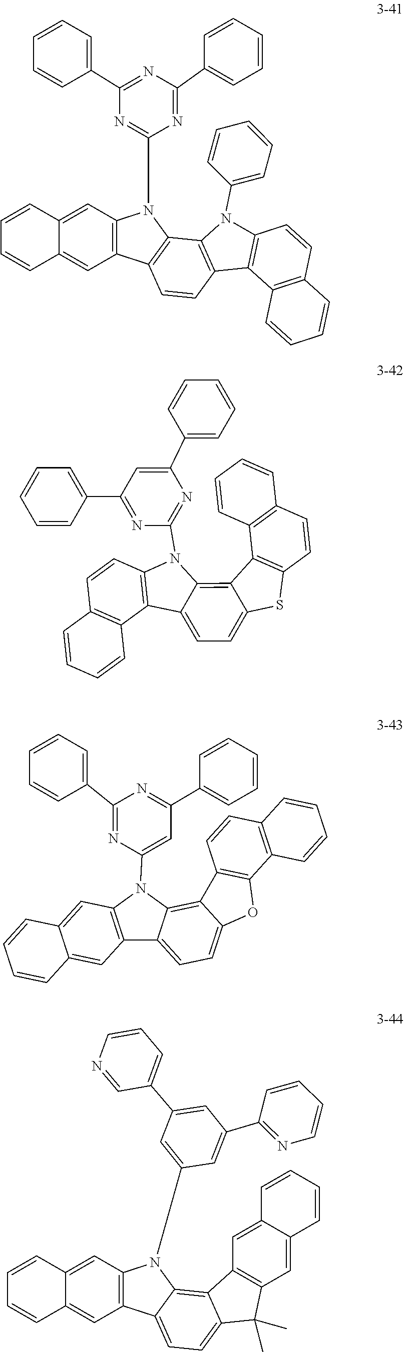 [SMILES: c1ccc(-c2nc(-c3ccccc3)nc(-n3c4cc5ccccc5cc4c4ccc5c6c7ccccc7ccc6n(-c6ccccc6)c5c43)n2)cc1 ; c1ccc(-c2cc(-c3ccccc3)nc(-n3c4ccc5ccccc5c4c4ccc5sc6ccc7ccccc7c6c5c43)n2)cc1 ; c1ccc(-c2cc(-n3c4cc5ccccc5cc4c4ccc5oc6c7ccccc7ccc6c5c43)nc(-c3ccccc3)n2)cc1 ; CC1(C)c2cc3ccccc3cc2-c2c1ccc1c3cc4ccccc4cc3n(-c3cc(-c4cccnc4)cc(-c4ccccn4)c3)c21]

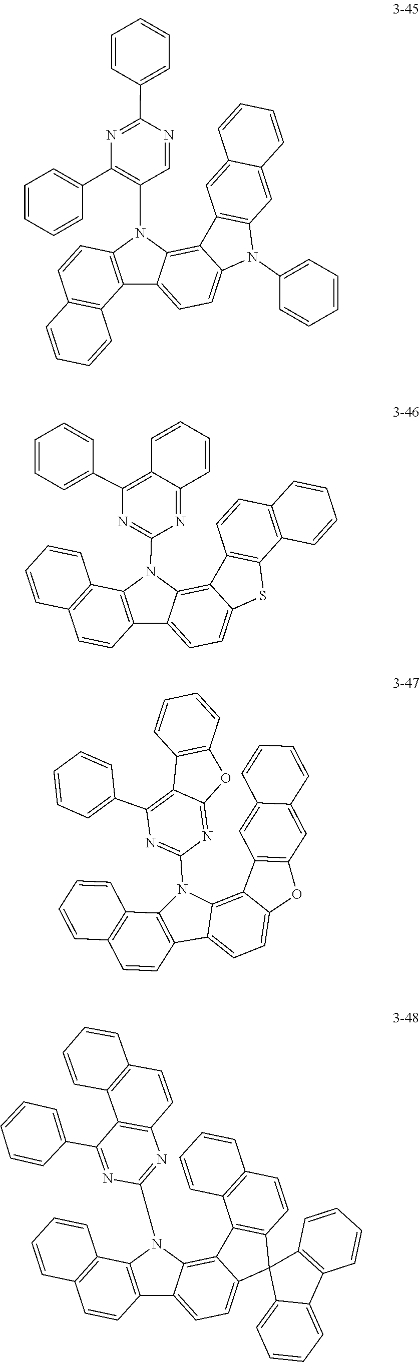

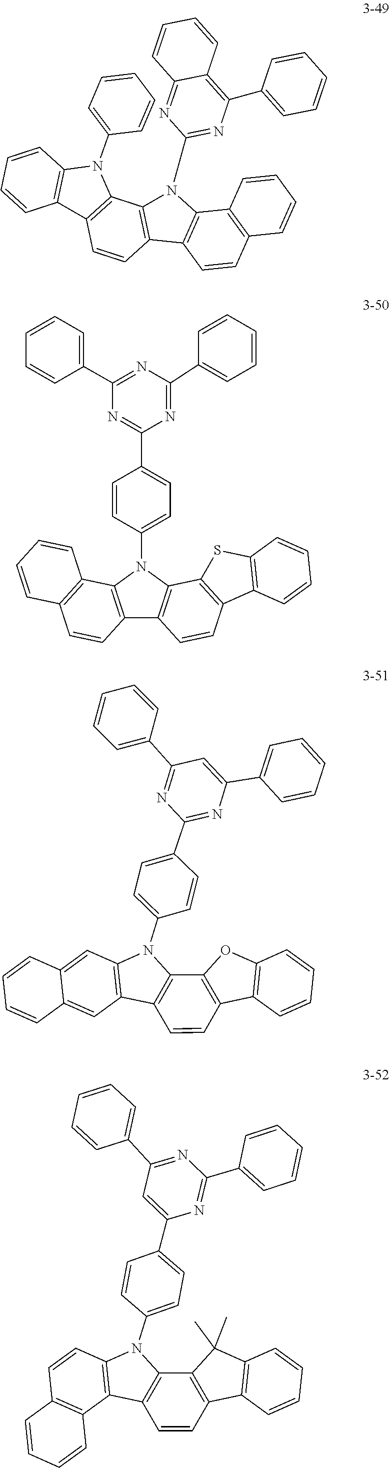 [SMILES: c1ccc(-c2nc(-n3c4c5ccccc5ccc4c4ccc5c6ccccc6n(-c6ccccc6)c5c43)nc3ccccc23)cc1 ; c1ccc(-c2nc(-c3ccccc3)nc(-c3ccc(-n4c5c6ccccc6ccc5c5ccc6c7ccccc7sc6c54)cc3)n2)cc1 ; c1ccc(-c2cc(-c3ccccc3)nc(-c3ccc(-n4c5cc6ccccc6cc5c5ccc6c7ccccc7oc6c54)cc3)n2)cc1 ; CC1(C)c2ccccc2-c2ccc3c4c5ccccc5ccc4n(-c4ccc(-c5cc(-c6ccccc6)nc(-c6ccccc6)n5)cc4)c3c21]

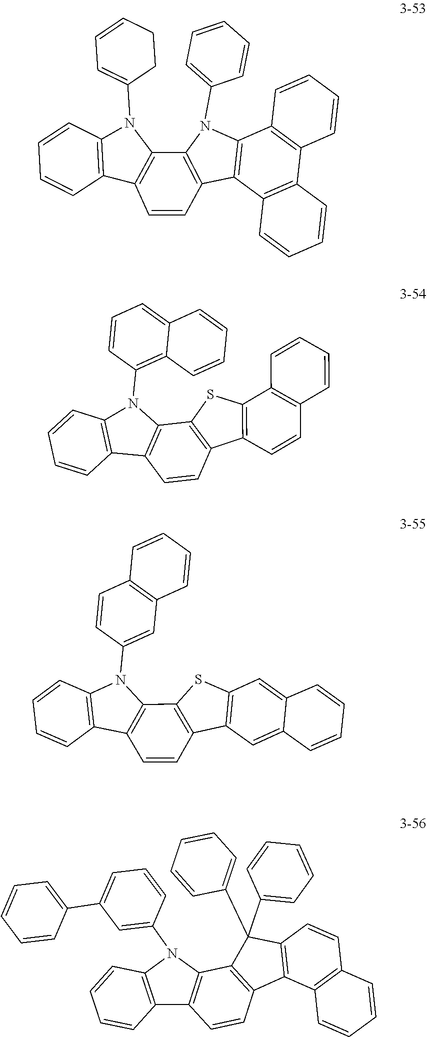 [SMILES: C1=CCCC(n2c3ccccc3c3ccc4c5c6ccccc6c6ccccc6c5n(-c5ccccc5)c4c32)=C1 ; c1ccc2c(-n3c4ccccc4c4ccc5c6ccc7ccccc7c6sc5c43)cccc2c1 ; c1ccc2cc(-n3c4ccccc4c4ccc5c6cc7ccccc7cc6sc5c43)ccc2c1 ; c1ccc(-c2cccc(-n3c4ccccc4c4ccc5c(c43)C(c3ccccc3)(c3ccccc3)c3ccc4ccccc4c3-5)c2)cc1]

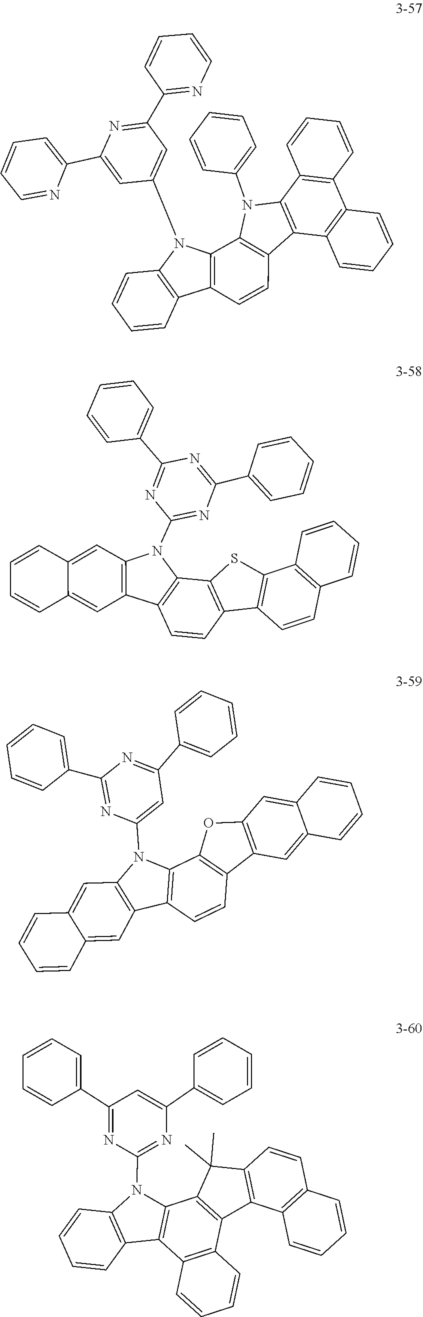 [SMILES: c1ccc(-n2c3c4ccccc4c4ccccc4c3c3ccc4c5ccccc5n(-c5cc(-c6ccccn6)nc(-c6ccccn6)c5)c4c32)cc1 ; c1ccc(-c2nc(-c3ccccc3)nc(-n3c4cc5ccccc5cc4c4ccc5c6ccc7ccccc7c6sc5c43)n2)cc1 ; c1ccc(-c2cc(-n3c4cc5ccccc5cc4c4ccc5c6cc7ccccc7cc6oc5c43)nc(-c3ccccc3)n2)cc1 ; CC1(C)c2ccc3ccccc3c2-c2c1c1c(c3ccccc23)c2ccccc2n1-c1nc(-c2ccccc2)cc(-c2ccccc2)n1]

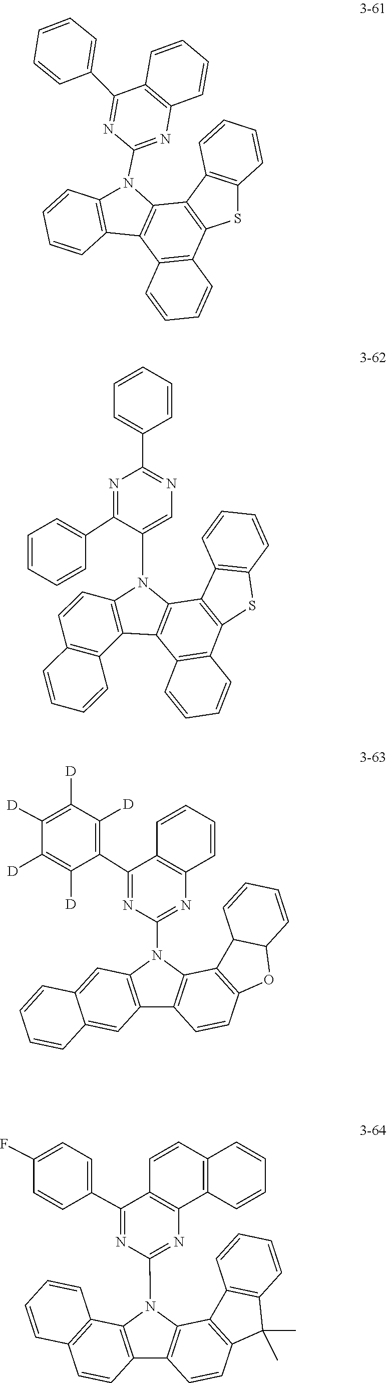 [SMILES: c1ccc(-c2nc(-n3c4ccccc4c4c5ccccc5c5sc6ccccc6c5c43)nc3ccccc23)cc1 ; c1ccc(-c2ncc(-n3c4ccc5ccccc5c4c4c5ccccc5c5sc6ccccc6c5c43)c(-c3ccccc3)n2)cc1 ; [2H]c1c([2H])c([2H])c(-c2nc(-n3c4cc5ccccc5cc4c4ccc5c(c43)C3C=CC=CC3O5)nc3ccccc23)c([2H])c1[2H] ; CC1(C)c2ccccc2-c2c1ccc1c3ccc4ccccc4c3n(-c3nc(-c4ccc(F)cc4)c4ccc5ccccc5c4n3)c21]

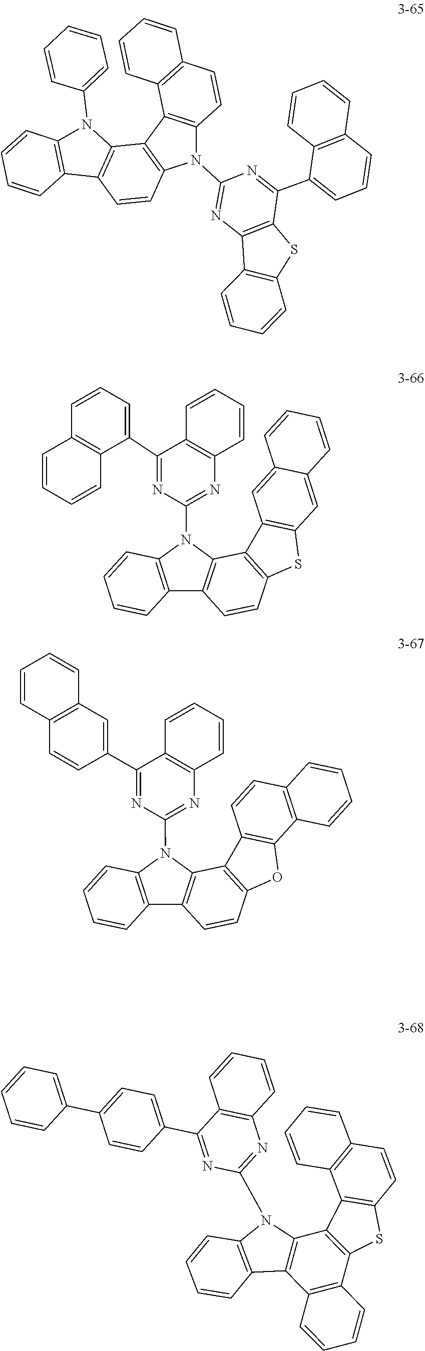 [SMILES: c1ccc(-n2c3ccccc3c3ccc4c(c5c6ccccc6ccc5n4-c4nc(-c5cccc6ccccc56)c5sc6ccccc6c5n4)c32)cc1 ; c1ccc2cc3c(cc2c1)sc1ccc2c4ccccc4n(-c4nc(-c5cccc6ccccc56)c5ccccc5n4)c2c13 ; c1ccc2cc(-c3nc(-n4c5ccccc5c5ccc6oc7c8ccccc8ccc7c6c54)nc4ccccc34)ccc2c1 ; c1ccc(-c2ccc(-c3nc(-n4c5ccccc5c5c6ccccc6c6sc7ccc8ccccc8c7c6c54)nc4ccccc34)cc2)cc1]

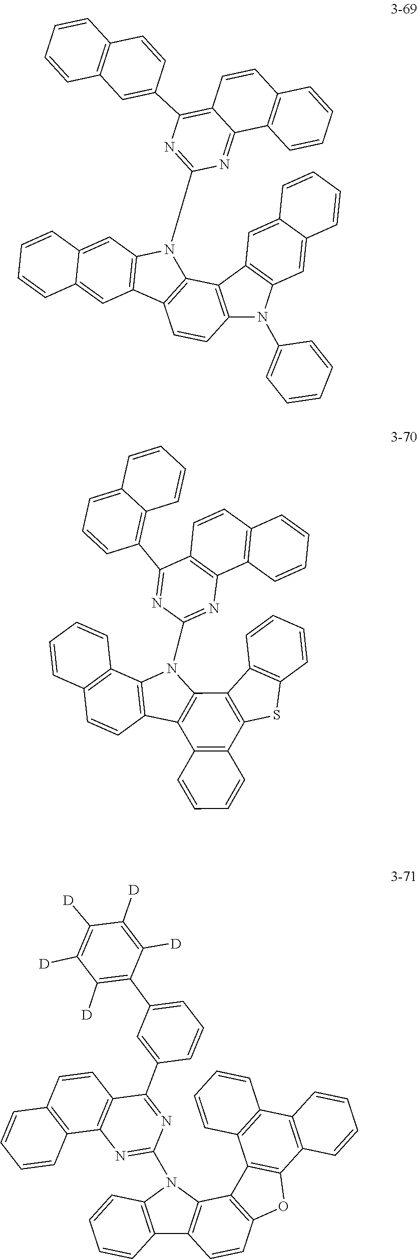 [SMILES: c1ccc(-n2c3cc4ccccc4cc3c3c2ccc2c4cc5ccccc5cc4n(-c4nc(-c5ccc6ccccc6c5)c5ccc6ccccc6c5n4)c23)cc1 ; c1ccc2c(-c3nc(-n4c5c6ccccc6ccc5c5c6ccccc6c6sc7ccccc7c6c54)nc4c3ccc3ccccc34)cccc2c1 ; [2H]c1c([2H])c([2H])c(-c2cccc(-c3nc(-n4c5ccccc5c5ccc6oc7c8ccccc8c8ccccc8c7c6c54)nc4c3ccc3ccccc34)c2)c([2H])c1[2H]]

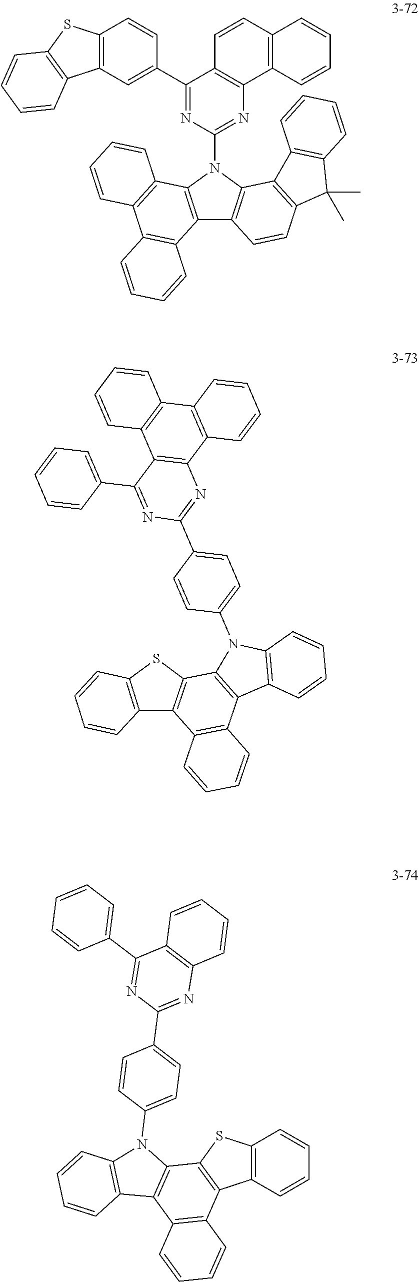 [SMILES: CC1(C)c2ccccc2-c2c1ccc1c3c4ccccc4c4ccccc4c3n(-c3nc(-c4ccc5sc6ccccc6c5c4)c4ccc5ccccc5c4n3)c21 ; c1ccc(-c2nc(-c3ccc(-n4c5ccccc5c5c6ccccc6c6c7ccccc7sc6c54)cc3)nc3c4ccccc4c4ccccc4c23)cc1 ; c1ccc(-c2nc(-c3ccc(-n4c5ccccc5c5c6ccccc6c6c7ccccc7sc6c54)cc3)nc3ccccc23)cc1]

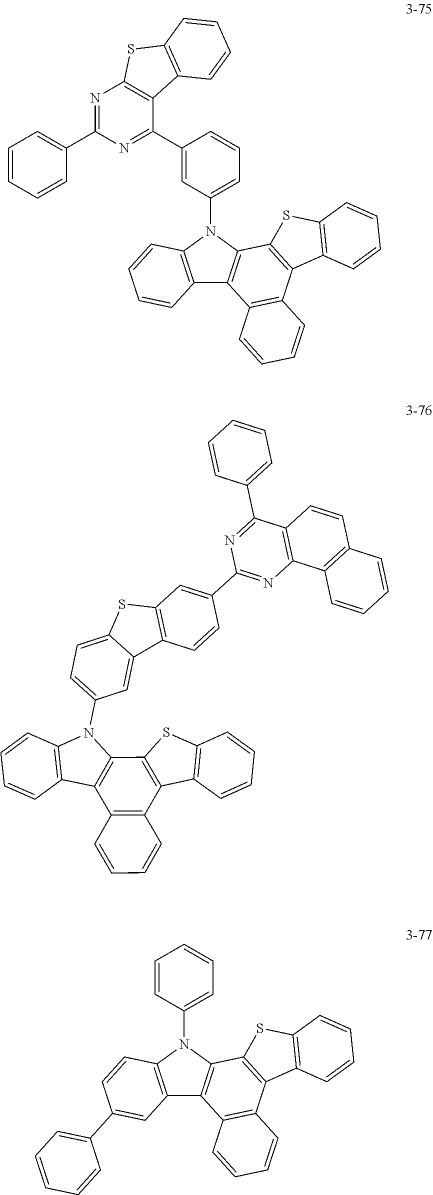 [SMILES: c1ccc(-c2nc(-c3cccc(-n4c5ccccc5c5c6ccccc6c6c7ccccc7sc6c54)c3)c3c(n2)sc2ccccc23)cc1 ; c1ccc(-c2nc(-c3ccc4c(c3)sc3ccc(-n5c6ccccc6c6c7ccccc7c7c8ccccc8sc7c65)cc34)nc3c2ccc2ccccc23)cc1 ; c1ccc(-c2ccc3c(c2)c2c4ccccc4c4c5ccccc5sc4c2n3-c2ccccc2)cc1]

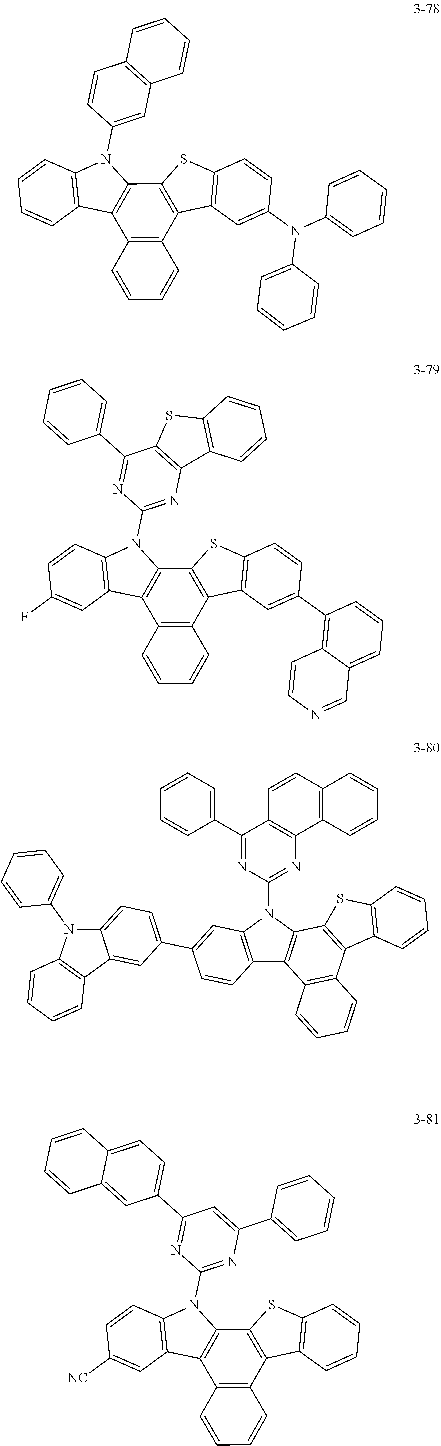 [SMILES: c1ccc(N(c2ccccc2)c2ccc3sc4c(c3c2)c2ccccc2c2c3ccccc3n(-c3ccc5ccccc5c3)c42)cc1 ; Fc1ccc2c(c1)c1c3ccccc3c3c4cc(-c5cccc6cnccc56)ccc4sc3c1n2-c1nc(-c2ccccc2)c2sc3ccccc3c2n1 ; c1ccc(-c2nc(-n3c4cc(-c5ccc6c(c5)c5ccccc5n6-c5ccccc5)ccc4c4c5ccccc5c5c6ccccc6sc5c43)nc3c2ccc2ccccc23)cc1 ; N#Cc1ccc2c(c1)c1c3ccccc3c3c4ccccc4sc3c1n2-c1nc(-c2ccccc2)cc(-c2ccc3ccccc3c2)n1]

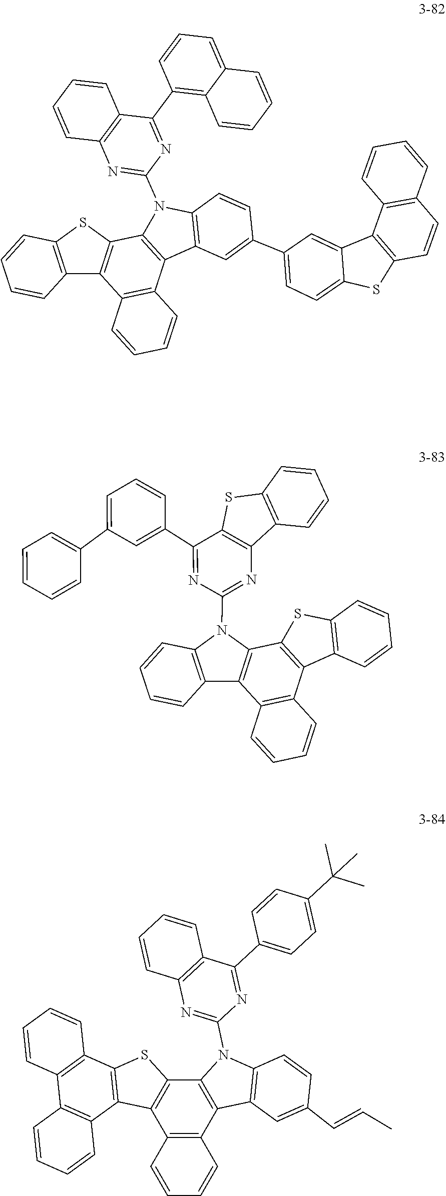 [SMILES: c1ccc2c(-c3nc(-n4c5ccc(-c6ccc7sc8ccc9ccccc9c8c7c6)cc5c5c6ccccc6c6c7ccccc7sc6c54)nc4ccccc34)cccc2c1 ; c1ccc(-c2cccc(-c3nc(-n4c5ccccc5c5c6ccccc6c6c7ccccc7sc6c54)nc4c3sc3ccccc34)c2)cc1 ; C/C=C/c1ccc2c(c1)c1c3ccccc3c3c(sc4c5ccccc5c5ccccc5c43)c1n2-c1nc(-c2ccc(C(C)(C)C)cc2)c2ccccc2n1]

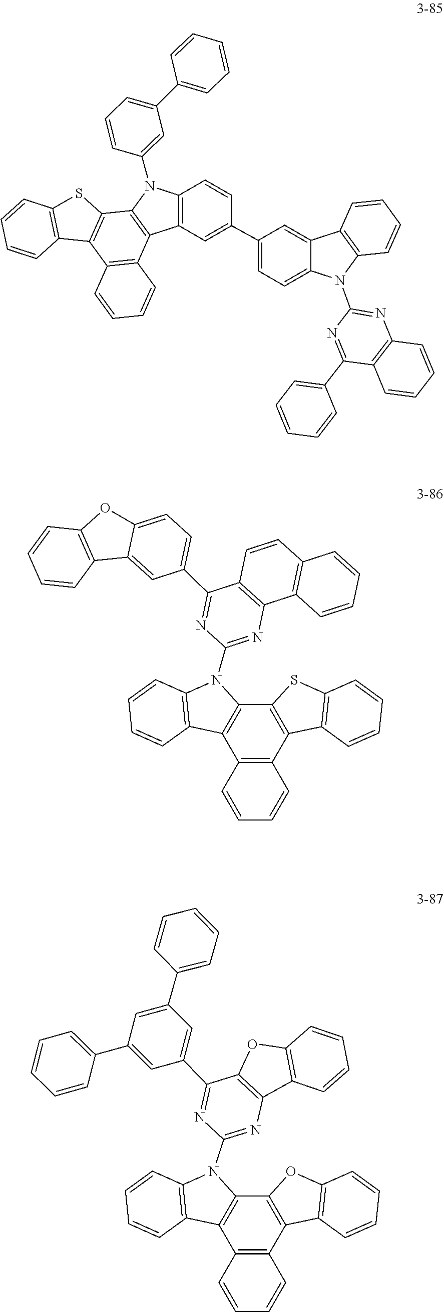 [SMILES: c1ccc(-c2cccc(-n3c4ccc(-c5ccc6c(c5)c5ccccc5n6-c5nc(-c6ccccc6)c6ccccc6n5)cc4c4c5ccccc5c5c6ccccc6sc5c43)c2)cc1 ; c1ccc2c(c1)ccc1c(-c3ccc4oc5ccccc5c4c3)nc(-n3c4ccccc4c4c5ccccc5c5c6ccccc6sc5c43)nc12 ; c1ccc(-c2cc(-c3ccccc3)cc(-c3nc(-n4c5ccccc5c5c6ccccc6c6c7ccccc7oc6c54)nc4c3oc3ccccc34)c2)cc1]

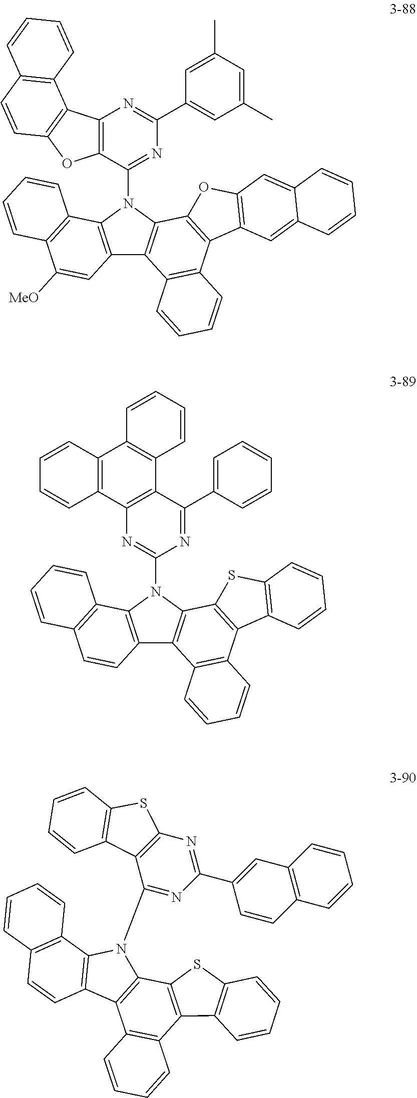 [SMILES: COc1cc2c3c4ccccc4c4c5cc6ccccc6cc5oc4c3n(-c3nc(-c4cc(C)cc(C)c4)nc4c3oc3ccc5ccccc5c34)c2c2ccccc12 ; c1ccc(-c2nc(-n3c4c5ccccc5ccc4c4c5ccccc5c5c6ccccc6sc5c43)nc3c4ccccc4c4ccccc4c23)cc1 ; c1ccc2cc(-c3nc(-n4c5c6ccccc6ccc5c5c6ccccc6c6c7ccccc7sc6c54)c4c(n3)sc3ccccc34)ccc2c1]

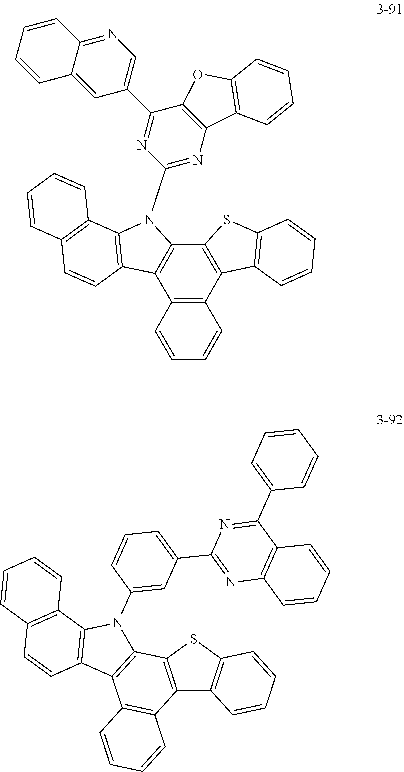 [SMILES: c1ccc2ncc(-c3nc(-n4c5c6ccccc6ccc5c5c6ccccc6c6c7ccccc7sc6c54)nc4c3oc3ccccc34)cc2c1 ; c1ccc(-c2nc(-c3cccc(-n4c5c6ccccc6ccc5c5c6ccccc6c6c7ccccc7sc6c54)c3)nc3ccccc23)cc1]

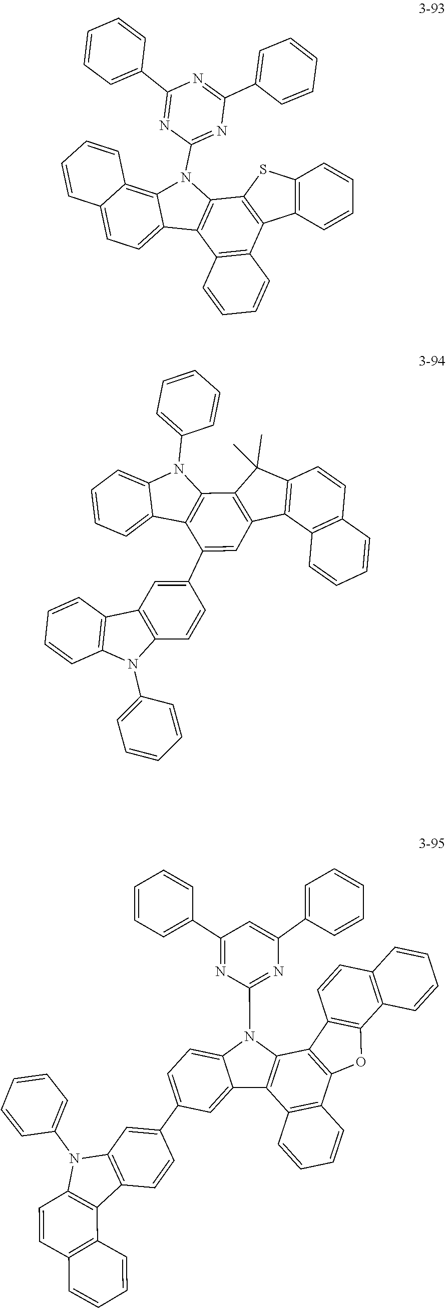 [SMILES: c1ccc(-c2nc(-c3ccccc3)nc(-n3c4c5ccccc5ccc4c4c5ccccc5c5c6ccccc6sc5c43)n2)cc1 ; CC1(C)c2ccc3ccccc3c2-c2cc(-c3ccc4c(c3)c3ccccc3n4-c3ccccc3)c3c4ccccc4n(-c4ccccc4)c3c21 ; c1ccc(-c2cc(-c3ccccc3)nc(-n3c4ccc(-c5ccc6c7c8ccccc8ccc7n(-c7ccccc7)c6c5)cc4c4c5ccccc5c5oc6c7ccccc7ccc6c5c43)n2)cc1]

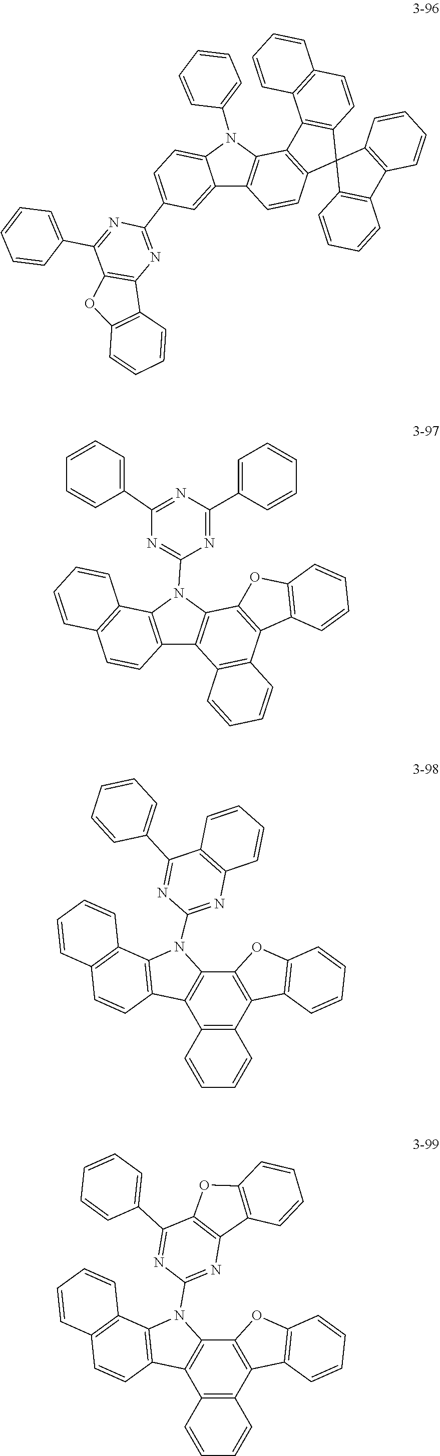 [SMILES: c1ccc(-c2nc(-c3ccc4c(c3)c3ccc5c(c3n4-c3ccccc3)-c3c(ccc4ccccc34)C53c4ccccc4-c4ccccc43)nc3c2oc2ccccc23)cc1 ; c1ccc(-c2nc(-c3ccccc3)nc(-n3c4c5ccccc5ccc4c4c5ccccc5c5c6ccccc6oc5c43)n2)cc1 ; c1ccc(-c2nc(-n3c4c5ccccc5ccc4c4c5ccccc5c5c6ccccc6oc5c43)nc3ccccc23)cc1 ; c1ccc(-c2nc(-n3c4c5ccccc5ccc4c4c5ccccc5c5c6ccccc6oc5c43)nc3c2oc2ccccc23)cc1]

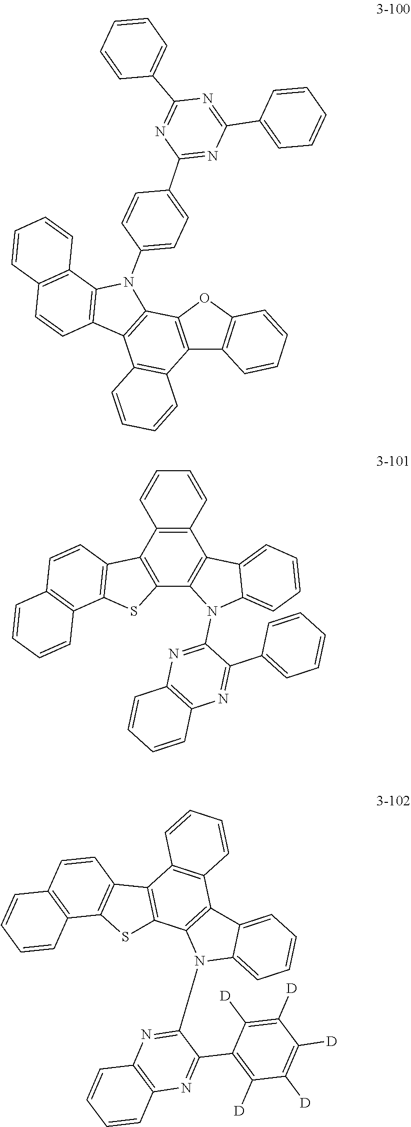

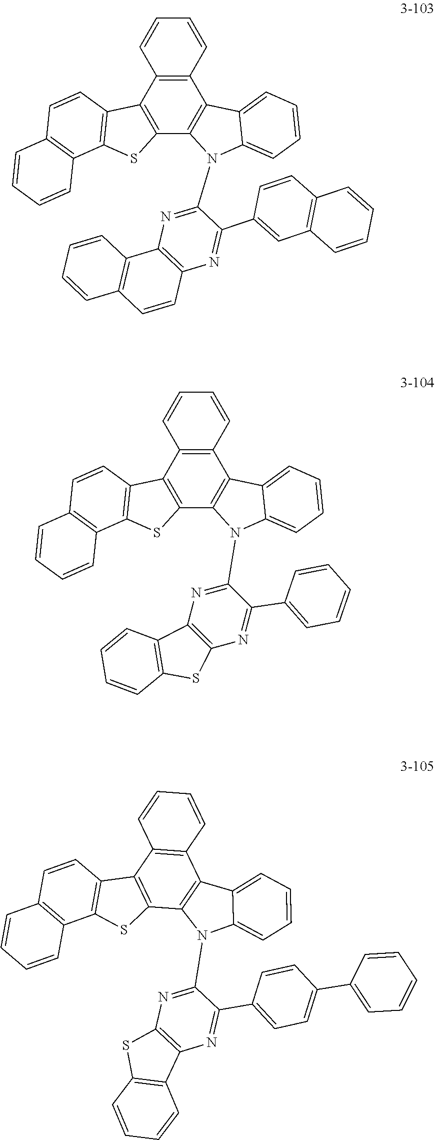 [SMILES: c1ccc2cc(-c3nc4ccc5ccccc5c4nc3-n3c4ccccc4c4c5ccccc5c5c6ccc7ccccc7c6sc5c43)ccc2c1 ; c1ccc(-c2nc3sc4ccccc4c3nc2-n2c3ccccc3c3c4ccccc4c4c5ccc6ccccc6c5sc4c32)cc1 ; c1ccc(-c2ccc(-c3nc4c(nc3-n3c5ccccc5c5c6ccccc6c6c7ccc8ccccc8c7sc6c53)sc3ccccc34)cc2)cc1]

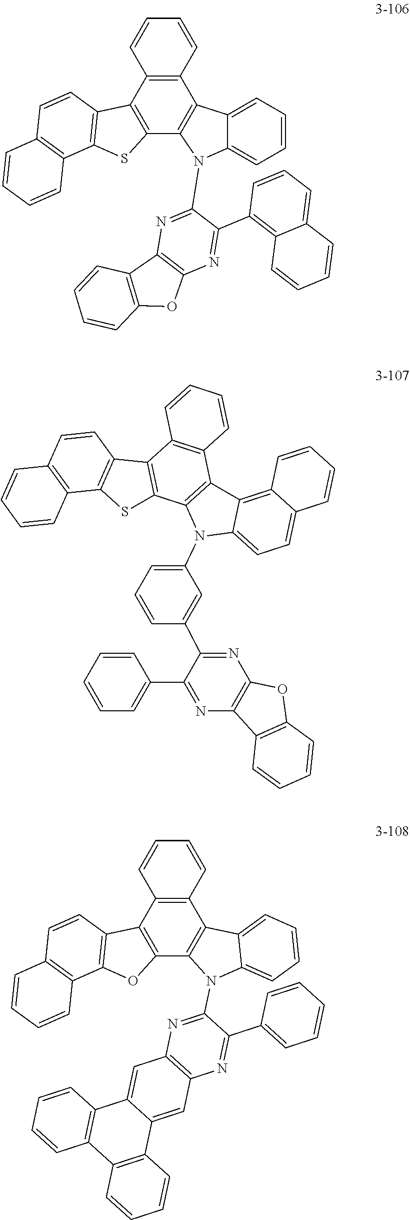 [SMILES: c1ccc2c(-c3nc4oc5ccccc5c4nc3-n3c4ccccc4c4c5ccccc5c5c6ccc7ccccc7c6sc5c43)cccc2c1 ; c1ccc(-c2nc3c(nc2-c2cccc(-n4c5ccc6ccccc6c5c5c6ccccc6c6c7ccc8ccccc8c7sc6c54)c2)oc2ccccc23)cc1 ; c1ccc(-c2nc3cc4c5ccccc5c5ccccc5c4cc3nc2-n2c3ccccc3c3c4ccccc4c4c5ccc6ccccc6c5oc4c32)cc1]

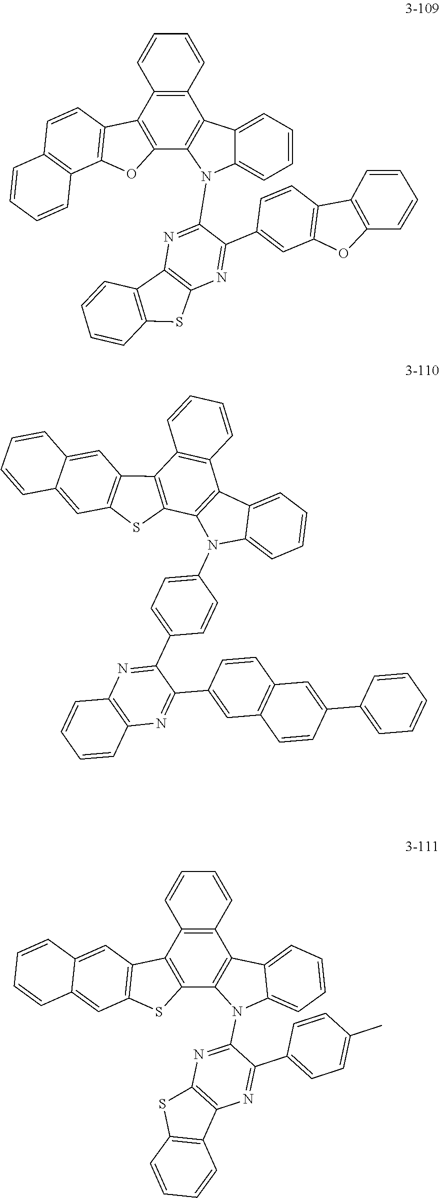 [SMILES: c1ccc2c(c1)ccc1c2oc2c1c1ccccc1c1c3ccccc3n(-c3nc4c(nc3-c3ccc5c(c3)oc3ccccc35)sc3ccccc34)c21 ; c1ccc(-c2ccc3cc(-c4nc5ccccc5nc4-c4ccc(-n5c6ccccc6c6c7ccccc7c7c8cc9ccccc9cc8sc7c65)cc4)ccc3c2)cc1 ; Cc1ccc(-c2nc3c(nc2-n2c4ccccc4c4c5ccccc5c5c6cc7ccccc7cc6sc5c42)sc2ccccc23)cc1]

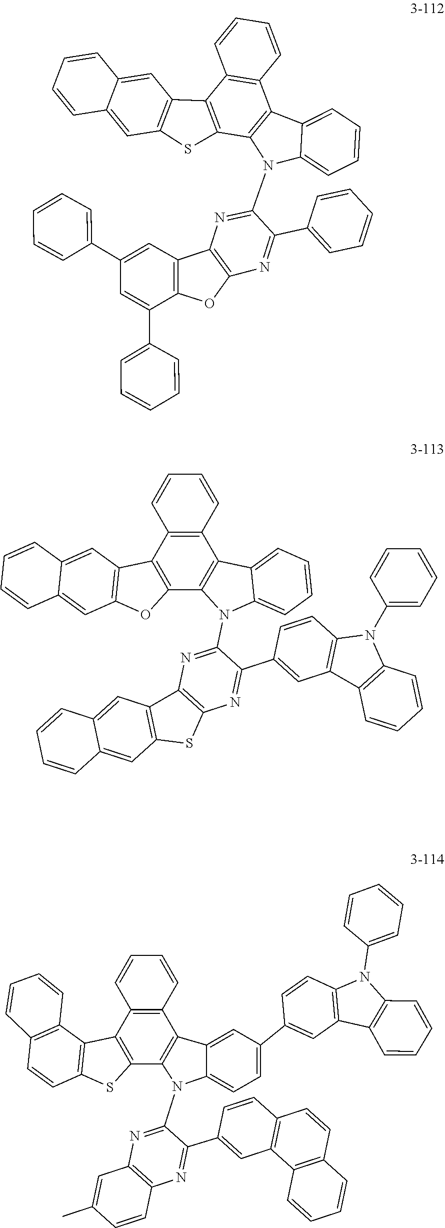 [SMILES: c1ccc(-c2cc(-c3ccccc3)c3oc4nc(-c5ccccc5)c(-n5c6ccccc6c6c7ccccc7c7c8cc9ccccc9cc8sc7c65)nc4c3c2)cc1 ; c1ccc(-n2c3ccccc3c3cc(-c4nc5sc6cc7ccccc7cc6c5nc4-n4c5ccccc5c5c6ccccc6c6c7cc8ccccc8cc7oc6c54)ccc32)cc1 ; Cc1ccc2nc(-c3ccc4ccc5ccccc5c4c3)c(-n3c4ccc(-c5ccc6c(c5)c5ccccc5n6-c5ccccc5)cc4c4c5ccccc5c5c(sc6ccc7ccccc7c65)c43)nc2c1]

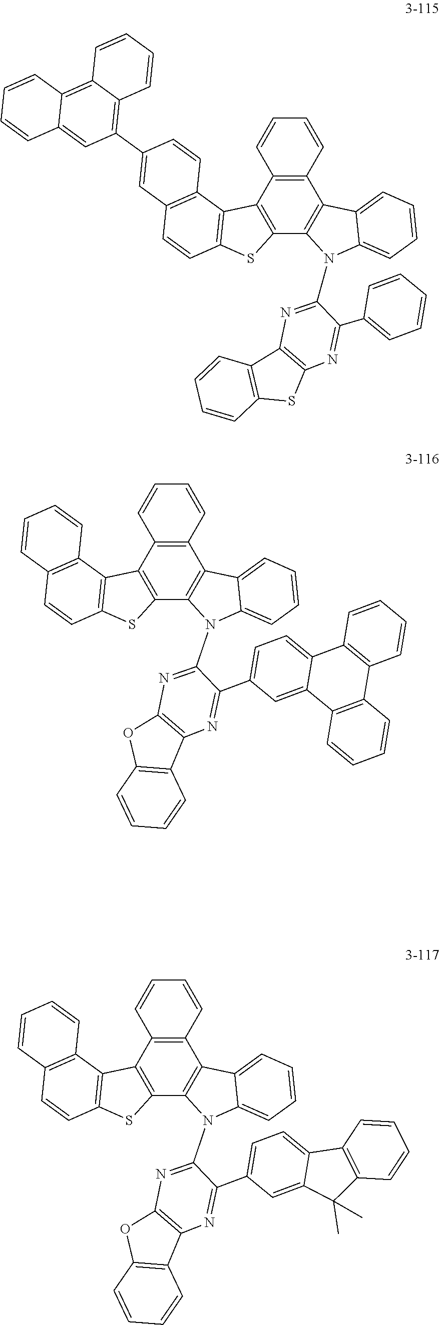 [SMILES: c1ccc(-c2nc3sc4ccccc4c3nc2-n2c3ccccc3c3c4ccccc4c4c(sc5ccc6cc(-c7cc8ccccc8c8ccccc78)ccc6c54)c32)cc1 ; c1ccc2c(c1)ccc1sc3c(c4ccccc4c4c5ccccc5n(-c5nc6oc7ccccc7c6nc5-c5ccc6c7ccccc7c7ccccc7c6c5)c34)c12 ; CC1(C)c2ccccc2-c2ccc(-c3nc4c(nc3-n3c5ccccc5c5c6ccccc6c6c(sc7ccc8ccccc8c76)c53)oc3ccccc34)cc21]

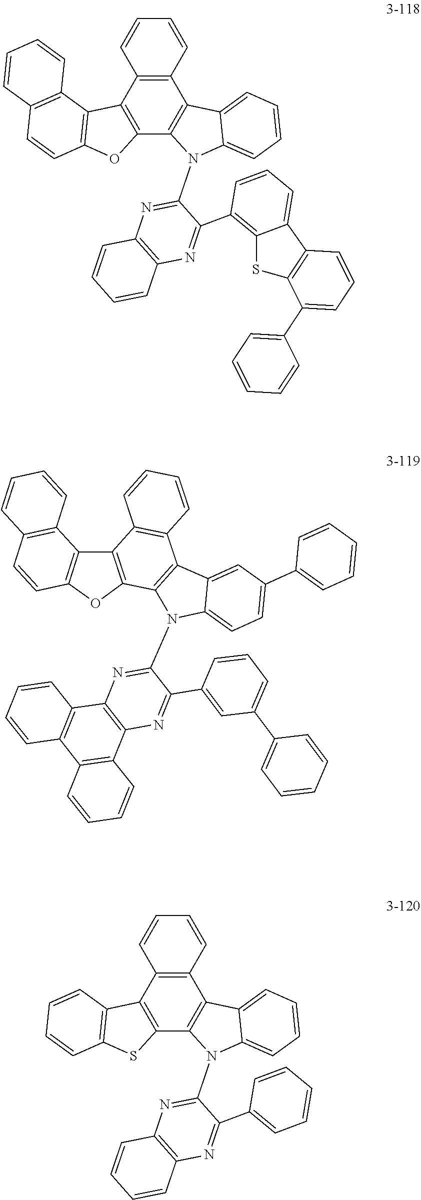 [SMILES: c1ccc(-c2cccc3c2sc2c(-c4nc5ccccc5nc4-n4c5ccccc5c5c6ccccc6c6c(oc7ccc8ccccc8c76)c54)cccc23)cc1 ; c1ccc(-c2cccc(-c3nc4c5ccccc5c5ccccc5c4nc3-n3c4ccc(-c5ccccc5)cc4c4c5ccccc5c5c(oc6ccc7ccccc7c65)c43)c2)cc1 ; c1ccc(-c2nc3ccccc3nc2-n2c3ccccc3c3c4ccccc4c4c5ccccc5sc4c32)cc1]

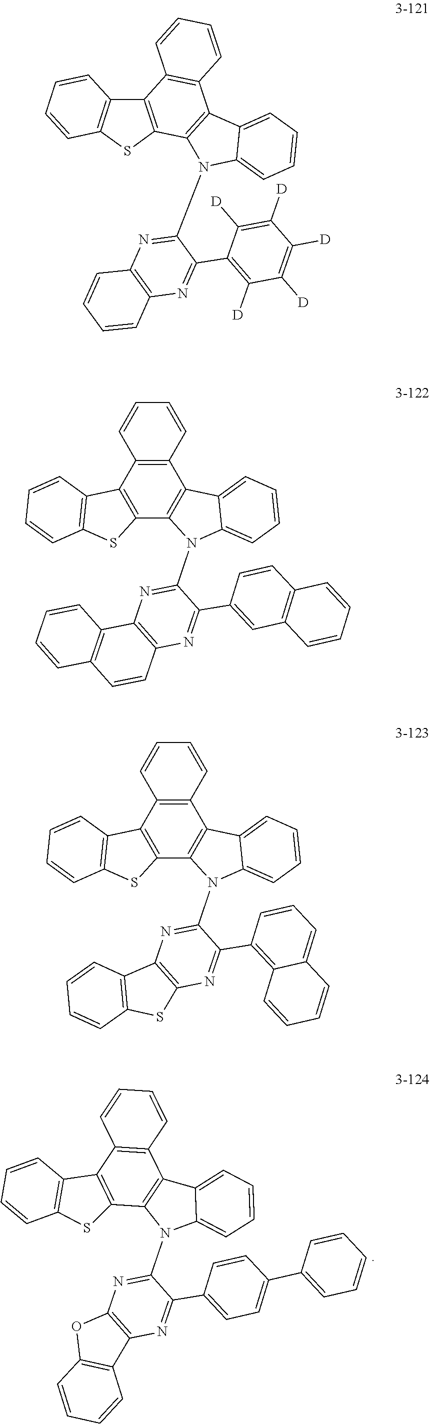 [SMILES: [2H]c1c([2H])c([2H])c(-c2nc3ccccc3nc2-n2c3ccccc3c3c4ccccc4c4c5ccccc5sc4c32)c([2H])c1[2H] ; c1ccc2cc(-c3nc4ccc5ccccc5c4nc3-n3c4ccccc4c4c5ccccc5c5c6ccccc6sc5c43)ccc2c1 ; c1ccc2c(-c3nc4sc5ccccc5c4nc3-n3c4ccccc4c4c5ccccc5c5c6ccccc6sc5c43)cccc2c1 ; c1ccc(-c2ccc(-c3nc4c(nc3-n3c5ccccc5c5c6ccccc6c6c7ccccc7sc6c53)oc3ccccc34)cc2)cc1]

13. The organic electric element of claim 1, further comprising at least one hole transporting band layer between the first electrode and the emitting layer, wherein the hole transporting band layer comprises a hole transport layer, an emitting auxiliary layer, or both, and the hole transporting band layer comprises a compound represented by Formula (1).

14. The organic electric element according to claim 1, wherein the compounds represented by Formula (1) and (2) are mixed in a ratio of 1:9 to 9:1 to be included in the emitting layer.

15. An organic electric element comprising: a first electrode; a second electrode; and an organic material layer disposed between the first electrode and the second electrode and comprising at least an emitting auxiliary layer and an emitting layer,
wherein the emitting auxiliary layer comprises a compound represented by Formula (1) and the emitting layer comprises a compound represented by Formula (2): 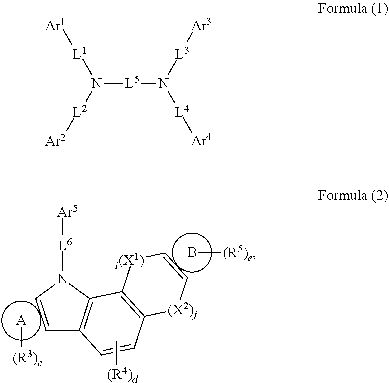
with the proviso that a compound of Formula (3-20) is excluded from Formula (1); 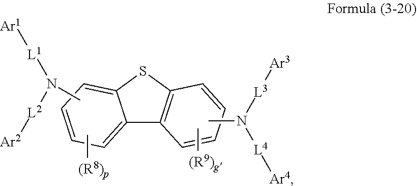
wherein Ar¹, Are, Ara, Ar⁴, Ar⁵, c, d, e, f′, g′, R³, R⁴, R⁵, R⁸, R⁹, L¹, L², L³, L⁴, L⁵, L⁶, A, B, j, X¹ and X² are the same as defined in claim 1.

16. A display device comprising the organic electric element of claim 1; and a control part driving the display device.

17. A display device comprising the organic electric element of claim 15; and a control part driving the display device.

18. A display device according to claim 16, wherein the organic electric element is at least one of an OLED, an organic solar cell, an organic photo conductor (OPC), organic transistor (organic TFT) and an element for monochromic or white illumination.

19. A display device according to claim 17, wherein the organic electric element is an OLED, an organic solar cell, an organic photo conductor (OPC), organic transistor (organic TFT), or an element for monochromic or white illumination.
